(19) United States
(12) Patent Application Publication
(10) Pub. No.: US 2024/0368203 A1
(43) Pub. Date: Nov. 7, 2024

(54) DEGRADATION OF (EGFR) BY CONJUGATION OF EGFR INHIBITORS WITH E3 LIGASE LIGAND AND METHODS OF USE

(71) Applicant: BeiGene, Ltd., Camana Bay (KY)

(72) Inventors: Bailin LEI, Beijing (CN); Huaqing LIU, Beijing (CN); Songzhe HAN, Beijing (CN); Yizhou ZHAO, Beijing (CN); Zhiwei WANG, Beijing (CN)

(73) Assignee: BeiGene, Ltd., Camana Bay (KY)

(21) Appl. No.: 18/771,529

(22) Filed: Jul. 12, 2024

Related U.S. Application Data
(63) Continuation of application No. PCT/CN2023/072825, filed on Jan. 18, 2023.

(30) Foreign Application Priority Data
Jan. 18, 2022 (WO) PCT/CN2022/072557

(51) Int. Cl. C07F 9/6558; A61K 31/675; A61P 35/00

(52) U.S. Cl. CPC C07F 9/65583; A61K 31/675; A61P 35/00

(57) ABSTRACT

Disclosed herein are novel bifunctional compounds formed by conjugating EGFR inhibitor moieties with E3 ligase Ligand moieties, which function to recruit targeted proteins to E3 ubiquitin ligase for degradation, and methods of preparation and uses thereof.

### CROSS-REFERENCE TO RELATED APPLICATIONS

This application is a continuation of International Application No. PCT/CN2023/072825, filed on Jan. 18, 2023, which claims priority to International Application No. PCT/CN2022/072557, filed on Jan. 18, 2022, the disclosures of each of which are hereby incorporated by reference in their entireties.

### FIELD OF THE INVENTION

Disclosed herein are novel bifunctional compounds formed by conjugating EGFR inhibitor moieties with E3 ligase Ligand moieties, which function to recruit targeted proteins to E3 ubiquitin ligase for degradation, and methods of preparation and uses thereof.

### BACKGROUND OF THE INVENTION

Proteolysis targeting chimera consists of two covalently linked protein-binding molecules: one capable of engaging an E3 ubiquitin ligase, and another that binds to the protein of interest (POI) a target meant for degradation (Sakamoto K M et al., Proc. Natl. Acad. Sci. 2001, 98: 8554-9.; Sakamoto K. M. et al., Methods Enzymol. 2005; 399:833-847.). Rather than inhibiting the target protein's enzymatic activity, recruitment of the E3 ligase to the specific unwanted proteins results in ubiquitination and subsequent degradation of the target protein by the proteasome. The whole process of ubiquitination and proteasomal degradation is known as the ubiquitin-proteasome pathway (UPP) (Ardley H. et al., Essays Biochem. 2005, 41, 15-30; Komander D. et al., Biochem. 2012, 81, 203-229; Grice G. L. et al., Cell Rep. 2015, 12, 545-553; Swatek K. N. et al., Cell Res. 2016, 26, 399-422). Proteasomes are protein complexes which degrade unneeded, misfolded or abnormal proteins into small peptides to maintain the health and productivity of the cells. Ubiquitin ligases, also called an E3 ubiquitin ligase, directly catalyze the transfer of ubiquitin from the E2 to the target protein for degradation. Although the human genome encodes over 600 putative E3 ligases, only a limited number of E3 ubiquitin ligases have been widely applied by small molecule PROTAC technology: cereblon (CRBN), Von Hippel-Lindau (VHL), mouse double minute 2 homologue (MDM2) and cellular inhibitor of apoptosis protein (clAP) (Philipp O. et al., Chem. Biol. 2017, 12, 2570-2578), recombinant Human Ring Finger Protein 114 (RNF114) (Spradlin, J. N. et al. Nat. Chem. Biol. 2019, 15, 747-755) and DDB1 And CUL4 Associated Factor 16 (DCAF16) (Zhang, X. et al. Nat. Chem. Biol. 2019, 15, 737-746). For example, cereblon (CRBN) forms an E3 ubiquitin ligase complex with damaged DNA binding protein 1 (DDB1) and Cullin-4A (CUL4A) to ubiquitinate a number of other proteins followed by the degradation via proteasomes. (Yi-An Chen, et al., Scientific Reports 2015, 5, 1-13). Immunomodulatory drugs (IMiDs), including thalidomide, lenalidomide, and pomalidomide, function as monovalent promoters of PPIs by binding to the cereblon (CRBN) subunit of the CRL4AC^{RB}N E3 ligase complex and recruiting neosubstrate proteins. (Matyskiela, M. E. et al., Nat Chem Biol 2018, 14, 981-987.) As a consequence, the ability of thalidomide, and its derivatives, to recruit CRBN has been widely applied in proteolysis-targeting chimeras (PROTACs) related studies (Christopher T. et al. ACS Chem. Biol. 2019, 14, 342-347.; Honorine L. et al, ACS Cent. Sci. 2016, 2, 927-934). PROTACs have great potential to eliminate protein targets that are “undruggable” by traditional inhibitors or are non-enzymatic proteins. (Chu T T. et al., Cell Chem Biol. 2016; 23:453-461. Qin C. et al., J Med Chem 2018; 61: 6685-6704. Winter G E. et al., Science 2015; 348:1376-1381.) In the recent years, PROTACs as useful modulators promote the selective degradation of a wide range of target proteins have been reported in antitumor studies. (Lu J. et al., Chem Biol. 2015; 22(6):755-763; Ottis P. et al., Chem Biol. 2017; 12(4):892-898.; Crews C. M. et al., J Med Chem. 2018; 61(2):403-404; Neklesa T. K. et al., Pharmacol Ther 2017, 174:138-144.; Cermakova K. et al., Molecules, 2018.23(8).; An S. et al., EBioMedicine, 2018.; Lebraud H. et al., Essays Biochem. 2017; 61(5): 517-527.; Sun Y. H. et al., Cell Res. 2018; 28:779-81; Toure M. et al., Angew Chem Int Ed Engl. 2016; 55(6):1966-1973; Yonghui Sun et al., Leukemia, volume 33, pages 2105-2110(2019); Shaodong Liu et al., Medicinal Chemistry Research, volume 29, pages 802-808(2020); and has been disclosed or discussed in patent publications, e.g., US20160045607, US20170008904, US20180050021, US20180072711, WO2002020740, WO2014108452, WO2016146985, WO2016149668, WO2016197032, WO2016197114, WO2017011590, WO2017030814, WO2017079267, WO2017182418, WO2017197036, WO2017197046, WO2017197051, WO2017197056, WO2017201449, and WO2018071606.

Epidermal growth factor receptor (EGFR) that belongs to the ErbB family is a transmembrane receptor tyrosine kinase (RTK), which plays a fundamental key role in cell proliferation, differentiation, and motility (Y. Yarden, et al., Nat. Rev. Mol. Cell Biol. 2001; 2:127-137.). Homo- or heterodimerization of EGFR and other ErbB family members activates cytoplasmic tyrosine kinase domains to initiate intracellular signaling. Overexpression or activating mutations of EGFR are associated with the development of many types of cancers, such as pancreatic cancer, breast cancer, glioblastoma multiforme, head and neck cancer, and non-small cell lung cancer (Yewale C., et al. Biomaterials. 2013, 34 (34): 8690-8707.). The activating mutations in the EGFR tyrosine kinase domain (L858R mutation and exon-19 deletion) have been identified as oncogenic drivers for NSCLC (Konduri, K., et al. Cancer Discovery 2016, 6 (6), 601-611.). The first-generation EGFR tyrosine kinase inhibitors (EGFR-TKIs) gefitinib and erlotinib have been approved for NSCLC patients with EGFR activation mutations (M. Maemondo, N. Engl. J Med. 362 (2010) 2380-2388.). Although most patients with EGFR mutant NSCLC respond to these therapies, patients typically develop resistance after an average of one year on treatment. There are several mechanisms of acquired resistance to gefitinib and erlotinib, including a secondary threonine 790 to methionine 790 mutation (T790M), is also called “gatekeeper” T790M mutation (Xu Y., et al. Cancer Biol Ther. 2010, 9 (8): 572-582.). Therefore, the second-generation EGFR-TKIs afatinib and the third-generation EGFR-TKIs osimertinib (AZD9291) were developed as irreversible EGFR inhibitors that bind to Cys797 for the treatment of patients with T790M mutation. In particular, osimertinib that largely spares WT EGFR has achieved greater clinical response rate in NSCLC patients with EGFR T790M. However, several recent studies have reported a tertiary Cys797 to Ser797 (C797S) point mutation with osimertinib clinical therapy (Thress K S, et al. Nat. Med. 2015, 21 (6): 560-562.). There is a need for drugs which can overcome EGFR (C797S) resistance obstacle in non-small cell lung cancer (NSCLC). EGFR-Targeting PROTACs serve as a potential strategy to overcome drug resistance mediated by these mutants, which has been disclosed or discussed in patent publications, e.g. WO2018119441, WO2019149922, WO2019183523, WO2019121562 and US20190106417.

Although, a number of EGFR-targeting PROTACs which were designed to degrade EGFR mutant proteins have been published (Zhang X., et al. Eur. J. Med. Chem. 2020, 192, 112199.; Zhang H, et al. Eur. J. Med. Chem. 2020, 189, 112061.; Lu X, Med. Res. Rev. 2018, 38(5):1550-1581. He K., et al. Bioorg. Med. Chem.

Lett. 2020, 15, 127167.). Most of the published molecules are based on first, second, and third generation of EGFR inhibitors. However, there were no data which showed those EGFR-Targeting PROTACs degrading all the main EGFR mutations, Such as Del19, L858R, Del19/T790M, L858R/T790M, L858R/C797S, Del19/C797S, Del19/T790M/C797S, L858R/T790M/C797S.

The present application provides novel bifunctional compounds and compositions for the treatment of serious diseases.

### SUMMARY OF THE INVENTION

One objective of the present invention is to provide compounds and derivatives formed by conjugating EGFR inhibitor moieties with E3 ligase Ligand moieties, which function to recruit targeted proteins to E3 ubiquitin ligase for degradation, and methods of preparation and uses thereof.

Aspect 1. A compound of Formula (I):

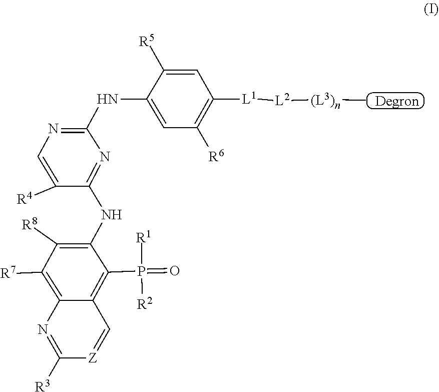

or a N-oxide thereof, or a pharmaceutically acceptable salt thereof, or a stereoisomer thereof, or a deuterated analog thereof, or a prodrug thereof,
wherein:
Z is N or CR^{z};
R^{z} is H, —C₁-C₈alkyl, C₃-C₈cycloalkyl or halogen;
R¹ and R² are each independently —C₁₋₈alkyl or —C₃₋₈cycloalkyl; each of said —C₁-C₈alkyl or C₃-C₈cycloalkyl is optionally substituted with at least one halogen;
R³ is hydrogen, —C₁-C₈alkyl, —C₂-C₈alkenyl, —C₂-C₈alkynyl, C₃-C₈cycloalkyl, halogen, 3- to 8-membered heterocyclyl, C₆-C₁₂aryl, 5- to 12-membered heteroaryl, —OR^{3a}, —SR^{3a}, —CN, —C(O)R^{3a}, —CO₂R^{3a}, —C(O)NR^{3a}R^{3b}, —NR^{3a}R^{3b}, —NR^{3a}COR^{3b}; each of said —C₁-C₈alkyl, —C₂-C₈alkenyl, —C₂-C₈alkynyl, C₃-C₈cycloalkyl, 3- to 8-membered heterocyclyl, C₆-C₁₂aryl or 5- to 12-membered heteroaryl is optionally substituted with 0 to 2 R^{3c};
R^{3a} and R^{3b} are each independently selected from hydrogen, —C₁-C₈alkyl, —C₁-C₈haloalkyl, —C₂-C₈alkenyl, —C₂-C₈alkynyl, C₁-C₈alkoxy-C₁-C₈alkyl-, C₃-C₈cycloalkyl, 3- to 8-membered heterocyclyl, C₆-C₁₂aryl or 5- to 12-membered heteroaryl;
R^{3c}, at each occurrence, is independently halogen, —OH, —CN, oxo (═O), —C₁-C₈alkyl, —C₂-C₈alkenyl, —C₂-C₈alkynyl, —C₃-C₈cycloalkyl, 3- to 8-membered heterocyclyl, —C₆-C₁₂aryl, or 5- to 12-membered heteroaryl;
R⁴ is halogen, methyl or methoxy;
R⁵ is hydrogen, halogen, —C₁-C₈alkyl, —NR^{5a}R^{5b}, —OR^{5a}, —SR^{5a}, C₃-C₈cycloalkyl, 3- to 8-membered heterocyclyl, C₆-C₁₂aryl, 5- to 12-membered heteroaryl, or CN; each of —C₁-C₈alkyl, C₃-C₈cycloalkyl, 3- to 8-membered heterocyclyl, C₆-C₁₂aryl, or 5- to 12-membered heteroaryl is optionally substituted with at least one R^{5c};
R^{5a} and R^{5b} are each independently selected from hydrogen, —C₁-C₈alkyl, —C₂-C₈alkenyl, —C₂-C₈alkynyl, C₃-C₈cycloalkyl, 3- to 8-membered heterocyclyl, C₆-C₁₂aryl, or 5- to 12-membered heteroaryl, each of said —C₁-C₈alkyl, —C₂-C₈alkenyl, —C₂-C₈alkynyl, C₃-C₈cycloalkyl, 3- to 8-membered heterocyclyl, C₆-C₁₂aryl, or 5- to 12-membered heteroaryl is optionally substituted with at least one substituent R^{5d};
R^{5c} and R^{5d} are each independently selected from halogen, hydroxy, —C₁-C₈alkyl, —C₁-C₈alkoxy, —C₂-C₈alkenyl, —C₂-C₈alkynyl, C₃-C₈cycloalkyl, 3- to 8-membered heterocyclyl, C₆-C₁₂aryl, or 5- to 12-membered heteroaryl;
R⁶ is hydrogen, halogen, —C₁-C₈alkyl, —NR^{6a}R^{6b}, —OR^{6a}, SR^{6a}, C₃-C₈cycloalkyl, 3- to 8-membered heterocyclyl, C₆-C₁₂aryl, 5- to 12-membered heteroaryl, or CN; each of —C₁-C₈alkyl, C₃-C₈cycloalkyl, 3- to 8-membered heterocyclyl, C₆-C₁₂aryl, or 5- to 12-membered heteroaryl is optionally substituted with at least one R^{6c};
R^{6a} and R^{6b} are each independently selected from hydrogen, —C₁-C₈alkyl, —C₂-C₈alkenyl, —C₂-C₈alkynyl, C₃-C₈cycloalkyl, 3- to 8-membered heterocyclyl, C₆-C₁₂aryl, or 5- to 12-membered heteroaryl, each of said —C₁-C₈alkyl, —C₂-C₈alkenyl, —C₂-C₈alkynyl, C₃-C₈cycloalkyl, 3- to 8-membered heterocyclyl, C₆-C₁₂aryl, or 5- to 12-membered heteroaryl is optionally substituted with at least one substituent R^{6d};
R^{6c} and R^{6d} are each independently selected from halogen, hydroxy, —C₁-C₈alkyl, —C₁-C₈alkoxy, —C₂-C₈alkenyl, —C₂-C₈alkynyl, C₃-C₈cycloalkyl, 3- to 8-membered heterocyclyl, C₆-C₁₂aryl, or 5- to 12-membered heteroaryl;
R⁷ and R⁸ are each independently selected from hydrogen, halogen, —C₁-C₈alkyl, —NR^{7a}R^{7b}, —OR^{7a}, —C₃-C₈cycloalkyl, 3- to 8-membered heterocyclyl, —C₆-C₁₂aryl, 5- to 12-membered heteroaryl, or —CN; each of —C₁-C₈alkyl, C₃-C₈cycloalkyl, 3- to 8-membered heterocyclyl, C₆-C₁₂aryl or 5- to 12-membered heteroaryl is optionally substituted with at least one substituent R^{7c}; or
R^{7a} and R^{7b} are each independently selected from hydrogen, —C₁-C₈alkyl, —C₂-C₈alkenyl, —C₂-C₈alkynyl, —C₃-C₈cycloalkyl, 3- to 8-membered heterocyclyl, —C₆-C₁₂aryl or 5- to 12-membered heteroaryl; each of said —C₁-C₈alkyl, —C₂-C₈alkenyl, —C₂-C₈alkynyl, C₃-C₈cycloalkyl, 3- to 8-membered heterocyclyl, C₆-C₁₂aryl or 5- to 12-membered heteroaryl is optionally substituted with at least one substituent R^{7d};
R^{7c} and R^{7d} are each independently halogen, hydroxy, —C₁-C₈alkyl, —C₁-C₈alkoxy, —C₂-C₈alkenyl, —C₂-C₈alkynyl, —C₃-C₈cycloalkyl, 3- to 8-membered heterocyclyl, —C₆-C₁₂aryl or 5- to 12-membered heteroaryl;

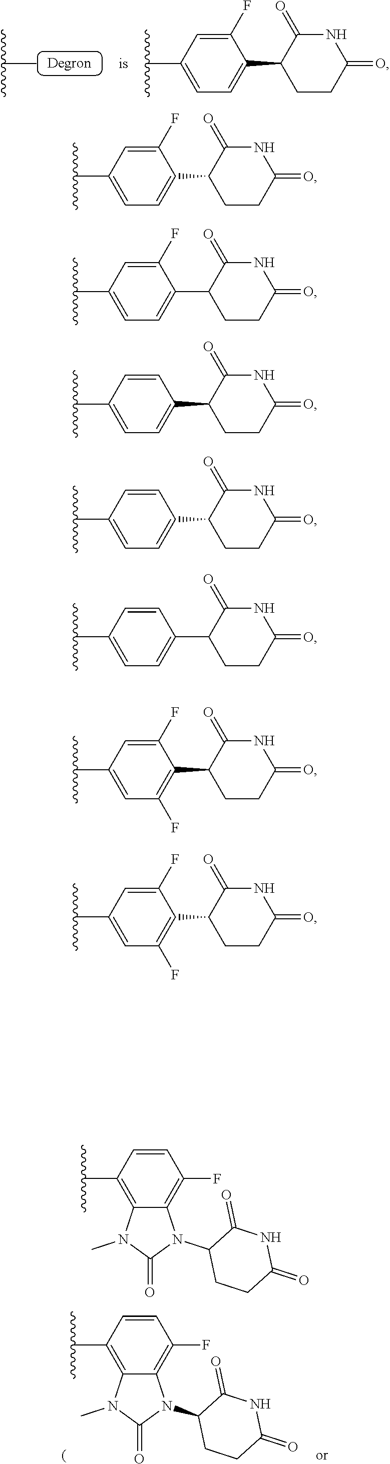

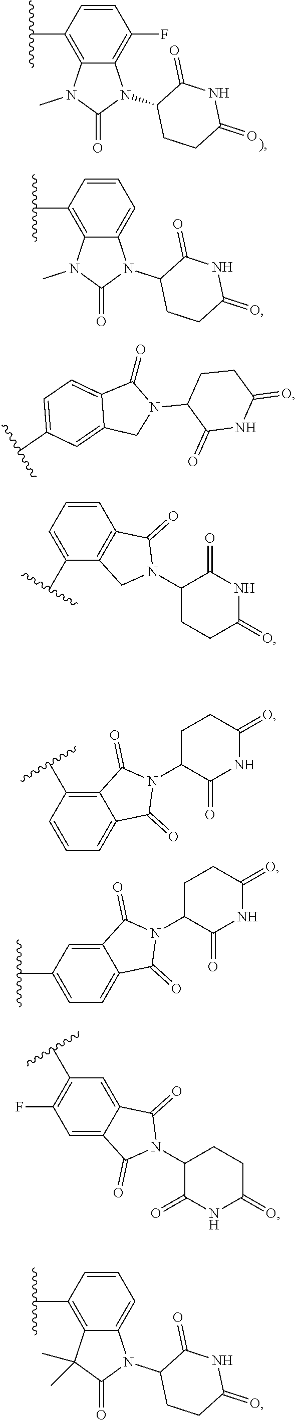

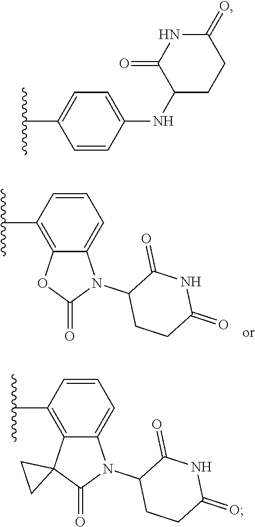

L¹ is

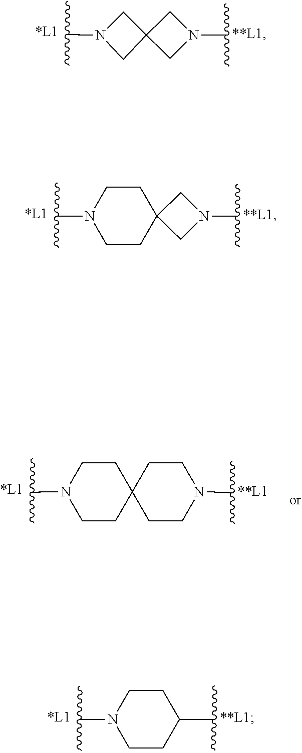 [SMILES: *N1CC2(C1)CN(*)C2 ; *N1CCC2(CC1)CN(*)C2 ; *N1CCC2(CC1)CCN(*)CC2 ; *C1CCN(*)CC1]

wherein *^{L1} refers to the position attached to the

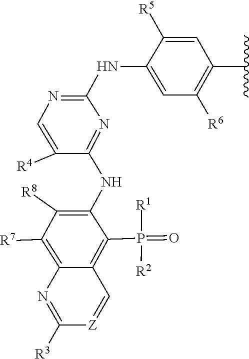

moiety, and **^{L1} refers to the position attached to the

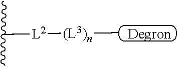

moiety;
L² is

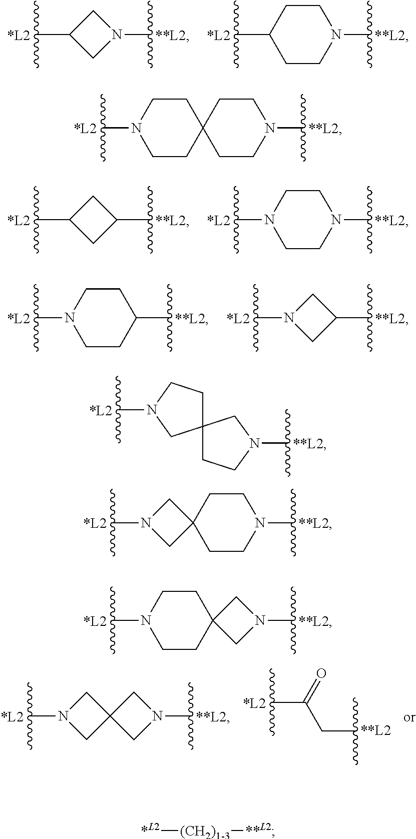

wherein *^{L2} refers to the position attached to the

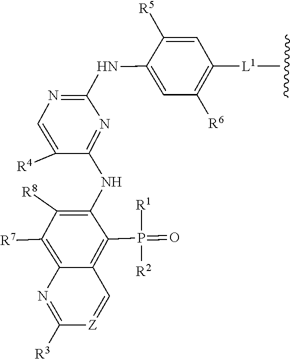

moiety, and **^{L2} refers to the position attached to the

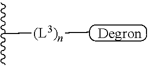

moiety;
L³ is

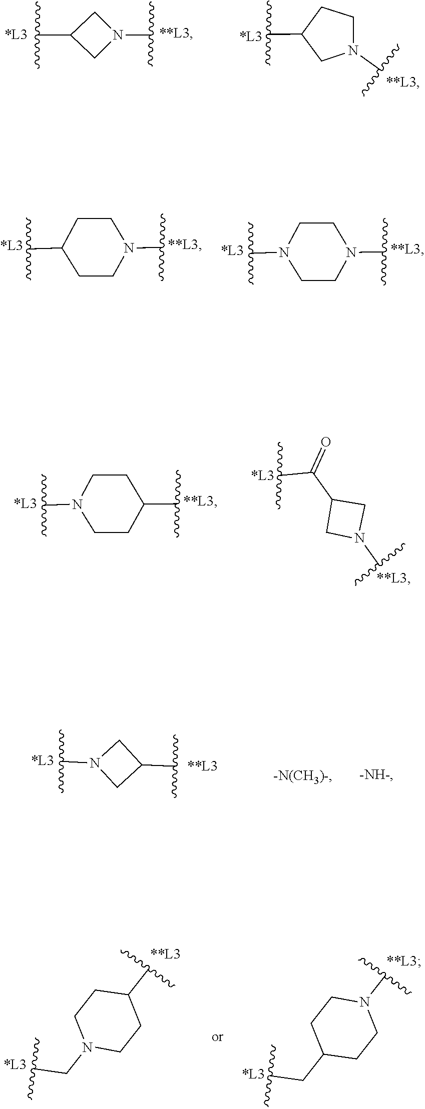

wherein *^{L3} refers to the position attached to the

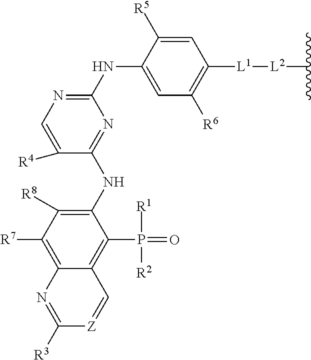

moiety, and **^{L3} refers to the position attached to the

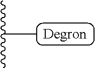

moiety;
each of said

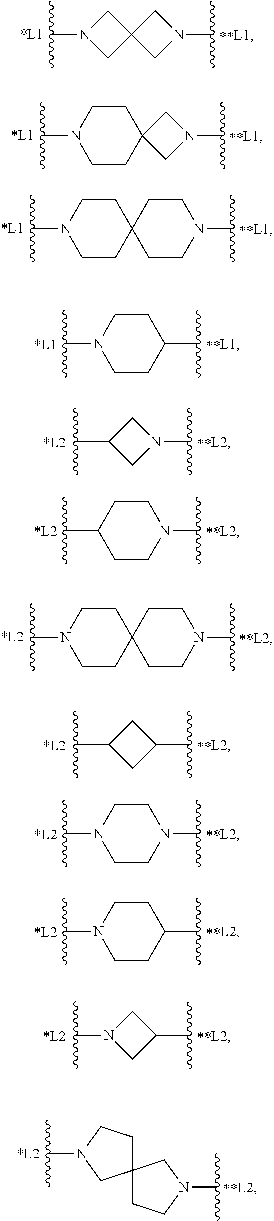

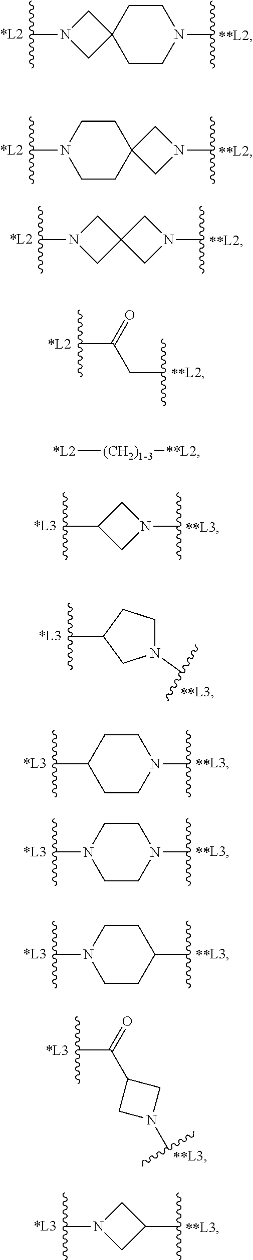

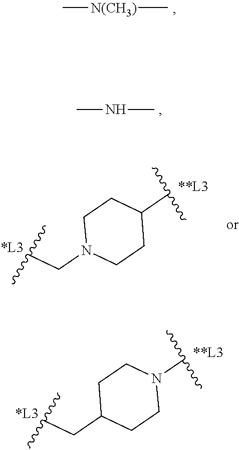

is optionally substituted with 0 to 2 R^{La};
R^{La}, at each occurrence, is independently halogen, —OH, —CN, oxo (═O), —C₁-C₈alkyl, —C₂-C₈alkenyl, —C₂-C₈alkynyl, —C₃-C₈cycloalkyl, 3- to 8-membered heterocyclyl, —C₆-C₁₂aryl, or 5- to 12-membered heteroaryl;
n is 0 or 1;
provided that the compound is not

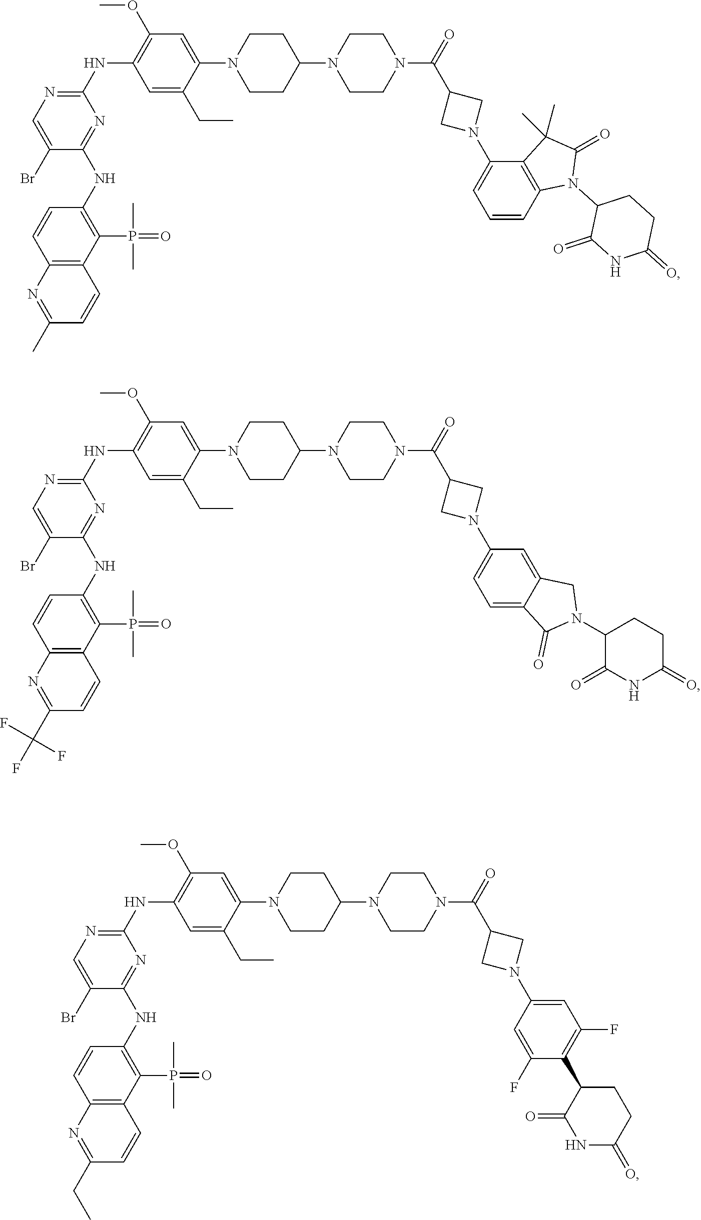 [SMILES: CCc1cc(Nc2ncc(Br)c(Nc3ccc4nc(C)ccc4c3P(C)(C)=O)n2)c(OC)cc1N1CCC(N2CCN(C(=O)C3CN(c4cccc5c4C(C)(C)C(=O)N5C4CCC(=O)NC4=O)C3)CC2)CC1 ; CCc1cc(Nc2ncc(Br)c(Nc3ccc4nc(C(F)(F)F)ccc4c3P(C)(C)=O)n2)c(OC)cc1N1CCC(N2CCN(C(=O)C3CN(c4ccc5c(c4)CN(C4CCC(=O)NC4=O)C5=O)C3)CC2)CC1 ; CCc1ccc2c(P(C)(C)=O)c(Nc3nc(Nc4cc(CC)c(N5CCC(N6CCN(C(=O)C7CN(c8cc(F)c([C@H]9CCC(=O)NC9=O)c(F)c8)C7)CC6)CC5)cc4OC)ncc3Br)ccc2n1]

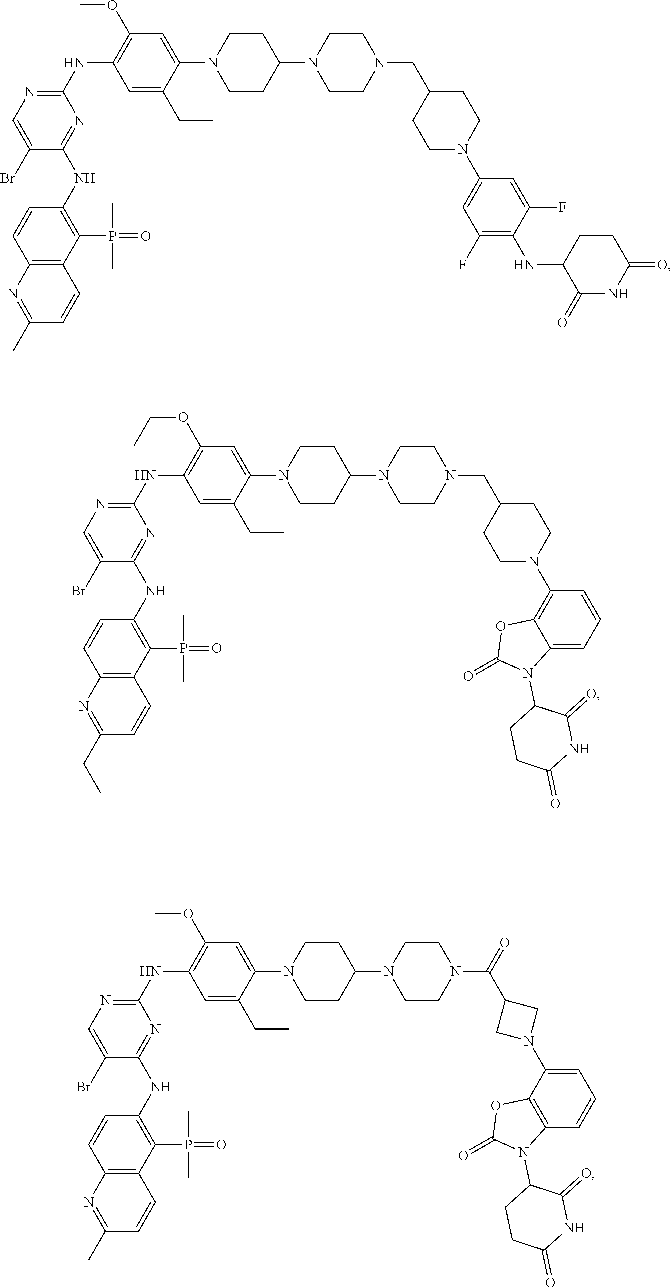 [SMILES: CCc1cc(Nc2ncc(Br)c(Nc3ccc4nc(C)ccc4c3P(C)(C)=O)n2)c(OC)cc1N1CCC(N2CCN(CC3CCN(c4cc(F)c(NC5CCC(=O)NC5=O)c(F)c4)CC3)CC2)CC1 ; CCOc1cc(N2CCC(N3CCN(CC4CCN(c5cccc6c5oc(=O)n6C5CCC(=O)NC5=O)CC4)CC3)CC2)c(CC)cc1Nc1ncc(Br)c(Nc2ccc3nc(CC)ccc3c2P(C)(C)=O)n1 ; CCc1cc(Nc2ncc(Br)c(Nc3ccc4nc(C)ccc4c3P(C)(C)=O)n2)c(OC)cc1N1CCC(N2CCN(C(=O)C3CN(c4cccc5c4oc(=O)n5C4CCC(=O)NC4=O)C3)CC2)CC1]

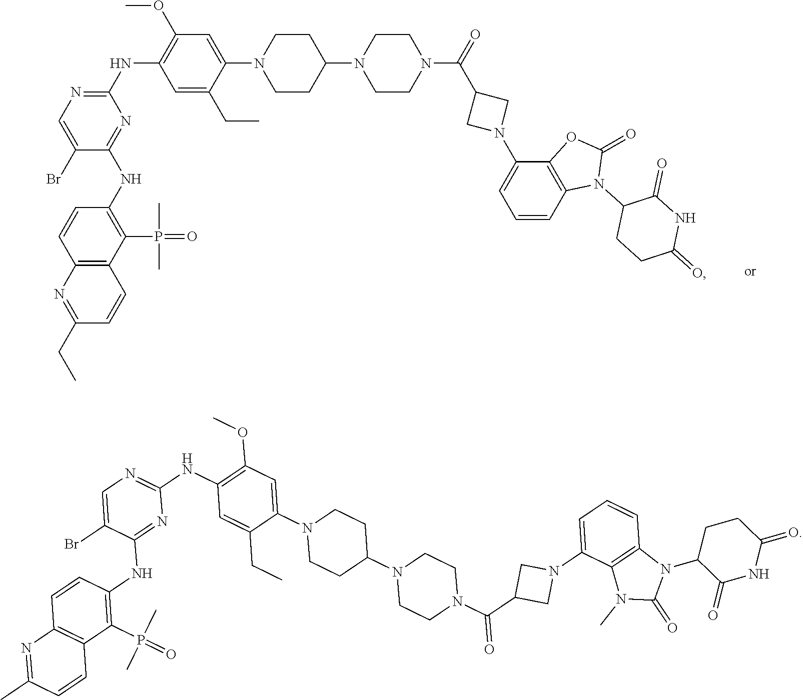 [SMILES: CCc1ccc2c(P(C)(C)=O)c(Nc3nc(Nc4cc(CC)c(N5CCC(N6CCN(C(=O)C7CN(c8cccc9c8oc(=O)n9C8CCC(=O)NC8=O)C7)CC6)CC5)cc4OC)ncc3Br)ccc2n1 ; CCc1cc(Nc2ncc(Br)c(Nc3ccc4nc(C)ccc4c3P(C)(C)=O)n2)c(OC)cc1N1CCC(N2CCN(C(=O)C3CN(c4cccc5c4n(C)c(=O)n5C4CCC(=O)NC4=O)C3)CC2)CC1]

In another embodiment, the compound is Formula (Ia):

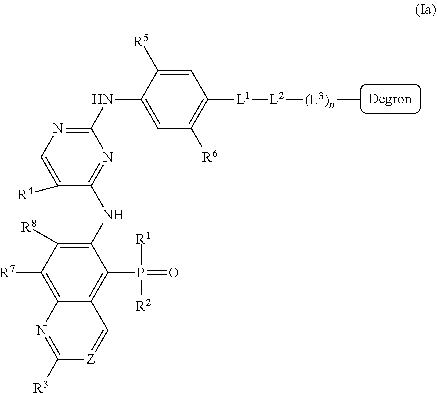

or a N-oxide thereof, or a pharmaceutically acceptable salt thereof, or a stereoisomer thereof, or a deuterated analog thereof, or a prodrug thereof,
wherein:
Z is N or CR^{z};
R^{z} is H, —C₁-C₈alkyl, C₃-C₈cycloalkyl or halogen;
R¹ and R² are each independently —C₁₋₈alkyl or —C₃₋₈cycloalkyl; each of said —C₁-C₈alkyl or C₃-C₈cycloalkyl is optionally substituted with at least one halogen;
R³ is hydrogen, —C₁-C₈alkyl, —C₂-C₈alkenyl, —C₂-C₈alkynyl, C₃-C₈cycloalkyl, halogen, 3- to 8-membered heterocyclyl, C₆-C₁₂aryl, 5- to 12-membered heteroaryl, —OR^{3a}, —SR^{3a}, —CN, —C(O)R^{3a}, —CO₂R^{3a}, —C(O)NR^{3a}R^{3b}, —NR^{3a}R^{3b}, —NR^{3a}COR^{3b}; each of said —C₁-C₈alkyl, —C₂-C₈alkenyl, —C₂-C₈alkynyl, C₃-C₈cycloalkyl, 3- to 8-membered heterocyclyl, C₆-C₁₂aryl or 5- to 12-membered heteroaryl is optionally substituted with 0 to 2 R^{3c};
R^{3a} and R^{3b} are each independently selected from hydrogen, —C₁-C₈alkyl, —C₁-C₈haloalkyl, —C₂-C₈alkenyl, —C₂-C₈alkynyl, C₁-C₈alkoxy-C₁-C₈alkyl-, C₃-C₈cycloalkyl, 3- to 8-membered heterocyclyl, C₆-C₁₂aryl or 5- to 12-membered heteroaryl;
R^{3c}, at each occurrence, is independently halogen, —OH, —CN, oxo (═O), —C₁-C₈alkyl, —C₂-C₈alkenyl, —C₂-C₈alkynyl, —C₃-C₈cycloalkyl, 3- to 8-membered heterocyclyl, —C₆-C₁₂aryl, or 5- to 12-membered heteroaryl;
R⁴ is halogen, methyl or methoxy;
R⁵ is hydrogen, halogen, —C₁-C₈alkyl, —NR^{5a}R^{5b}, —OR^{5a}, —SR^{5a}, C₃-C₈cycloalkyl, 3- to 8-membered heterocyclyl, C₆-C₁₂aryl, 5- to 12-membered heteroaryl, or CN; each of —C₁-C₈alkyl, C₃-C₈cycloalkyl, 3- to 8-membered heterocyclyl, C₆-C₁₂aryl, or 5- to 12-membered heteroaryl is optionally substituted with at least one R^{5c};
R^{5a} and R^{5b} are each independently selected from hydrogen, —C₁-C₈alkyl, —C₂-C₈alkenyl, —C₂-C₈alkynyl, C₃-C₈cycloalkyl, 3- to 8-membered heterocyclyl, C₆-C₁₂aryl, or 5- to 12-membered heteroaryl, each of said —C₁-C₈alkyl, —C₂-C₈alkenyl, —C₂-C₈alkynyl, C₃-C₈cycloalkyl, 3- to 8-membered heterocyclyl, C₆-C₁₂aryl, or 5- to 12-membered heteroaryl is optionally substituted with at least one substituent R^{5d};
R^{5c} and R^{5d} are each independently selected from halogen, hydroxy, —C₁-C₈alkyl, —C₁-C₈alkoxy, —C₂-C₈alkenyl, —C₂-C₈alkynyl, C₃-C₈cycloalkyl, 3- to 8-membered heterocyclyl, C₆-C₁₂aryl, or 5- to 12-membered heteroaryl;
R⁶ is hydrogen, halogen, —C₁-C₈alkyl, —NR^{6a}R^{6b}, —OR^{6a}, —SR^{6a}, C₃-C₈cycloalkyl, 3- to 8-membered heterocyclyl, C₆-C₁₂aryl, 5- to 12-membered heteroaryl, or CN; each of —C₁-C₈alkyl, C₃-C₈cycloalkyl, 3- to 8-membered heterocyclyl, C₆-C₁₂aryl, or 5- to 12-membered heteroaryl is optionally substituted with at least one R^{6c};
R^{6a} and R^{6b} are each independently selected from hydrogen, —C₁-C₈alkyl, —C₂-C₈alkenyl, —C₂-C₈alkynyl, C₃-C₈cycloalkyl, 3- to 8-membered heterocyclyl, C₆-C₁₂aryl, or 5- to 12-membered heteroaryl, each of said —C₁-C₈alkyl, —C₂-C₈alkenyl, —C₂-C₈alkynyl, C₃-C₈cycloalkyl, 3- to 8-membered heterocyclyl, C₆-C₁₂aryl, or 5- to 12-membered heteroaryl is optionally substituted with at least one substituent R^{6d};
R^{6c} and R^{6d} are each independently selected from halogen, hydroxy, —C₁-C₈alkyl, —C₁-C₈alkoxy, —C₂-C₈alkenyl, —C₂-C₈alkynyl, C₃-C₈cycloalkyl, 3- to 8-membered heterocyclyl, C₆-C₁₂aryl, or 5- to 12-membered heteroaryl;
R⁷ and R⁸ are each independently selected from hydrogen, halogen, —C₁-C₈alkyl, —NR^{7a}R^{7b}, —OR^{7a}, —C₃-C₈cycloalkyl, 3- to 8-membered heterocyclyl, —C₆-C₁₂aryl, 5- to 12-membered heteroaryl, or —CN; each of —C₁-C₈alkyl, C₃-C₈cycloalkyl, 3- to 8-membered heterocyclyl, C₆-C₁₂aryl or 5- to 12-membered heteroaryl is optionally substituted with at least one substituent R^{7c}; or
R^{7a} and R^{7b} are each independently selected from hydrogen, —C₁-C₈alkyl, —C₂-C₈alkenyl, —C₂-C₈alkynyl, —C₃-C₈cycloalkyl, 3- to 8-membered heterocyclyl, —C₆-C₁₂aryl or 5- to 12-membered heteroaryl; each of said —C₁-C₈alkyl, —C₂-C₈alkenyl, —C₂-C₈alkynyl, C₃-C₈cycloalkyl, 3- to 8-membered heterocyclyl, C₆-C₁₂aryl or 5- to 12-membered heteroaryl is optionally substituted with at least one substituent R^{7d};
R^{7c} and R^{7d} are each independently halogen, hydroxy, —C₁-C₈alkyl, —C₁-C₈alkoxy, —C₂-C₈alkenyl, —C₂-C₈alkynyl, —C₃-C₈cycloalkyl, 3- to 8-membered heterocyclyl, —C₆-C₁₂aryl or 5- to 12-membered heteroaryl;

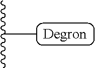

selected from

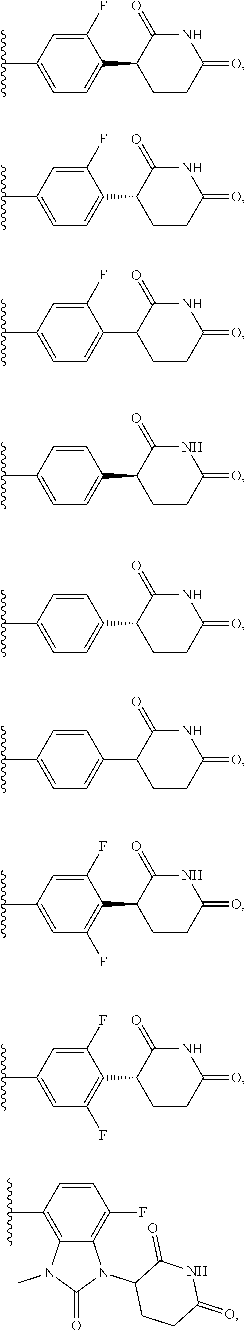

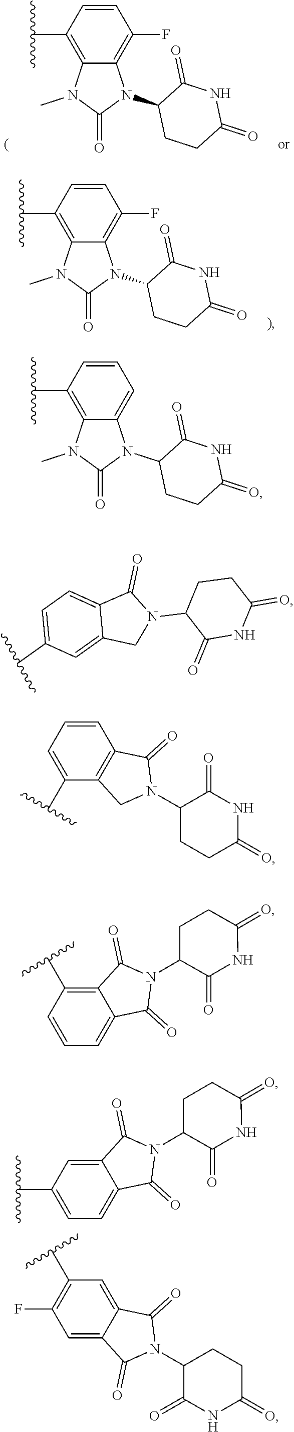

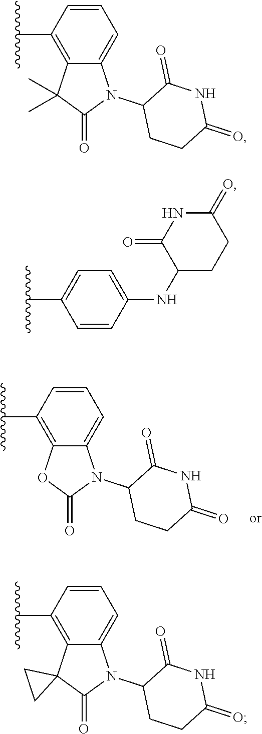 [SMILES: *c1cccc2c1C(C)(C)C(=O)N2C1CCC(=O)NC1=O ; *c1ccc(NC2CCC(=O)NC2=O)cc1 ; *c1cccc2c1oc(=O)n2C1CCC(=O)NC1=O ; *c1cccc2c1C1(CC1)C(=O)N2C1CCC(=O)NC1=O]

L¹ is

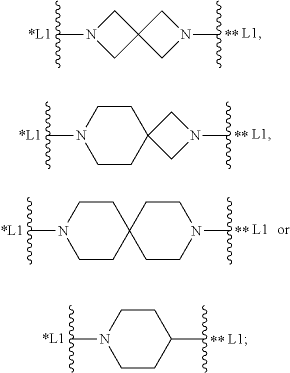

wherein *^{L1} refers to the position attached to the

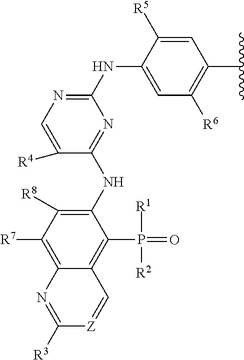

moiety, and **^{L1} refers to the position attached to the

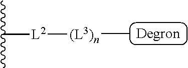

moiety;
L² is

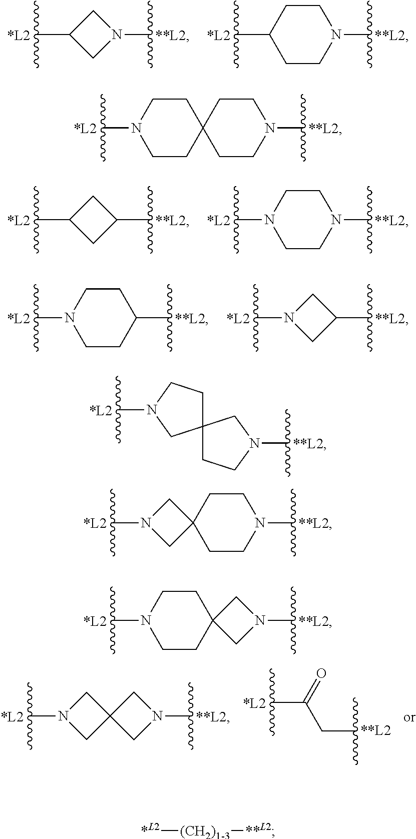

wherein *^{L2} refers to the position attached to the

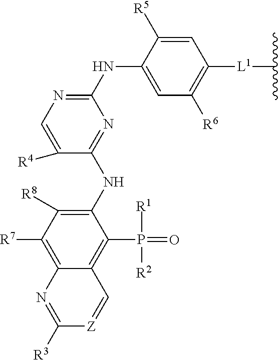

moiety, and **^{L2} refers to the position attached to the

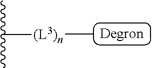

moiety;
L³ is

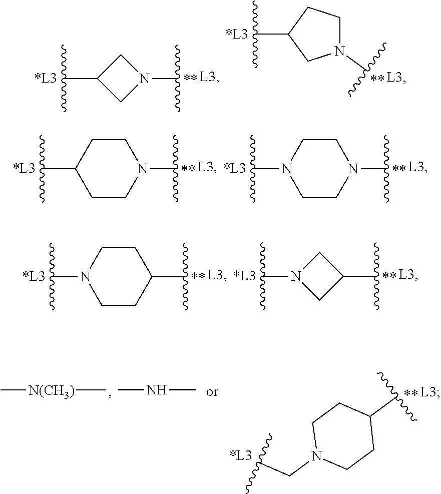

wherein *^{L3} refers to the position attached to the

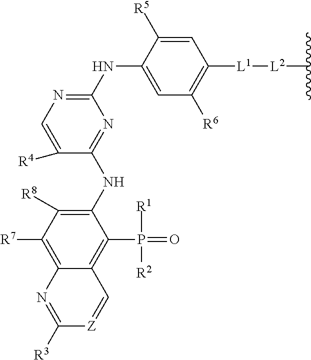

moiety, and **^{L3} refers to the position attached to the

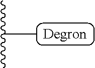

moiety;
each of said

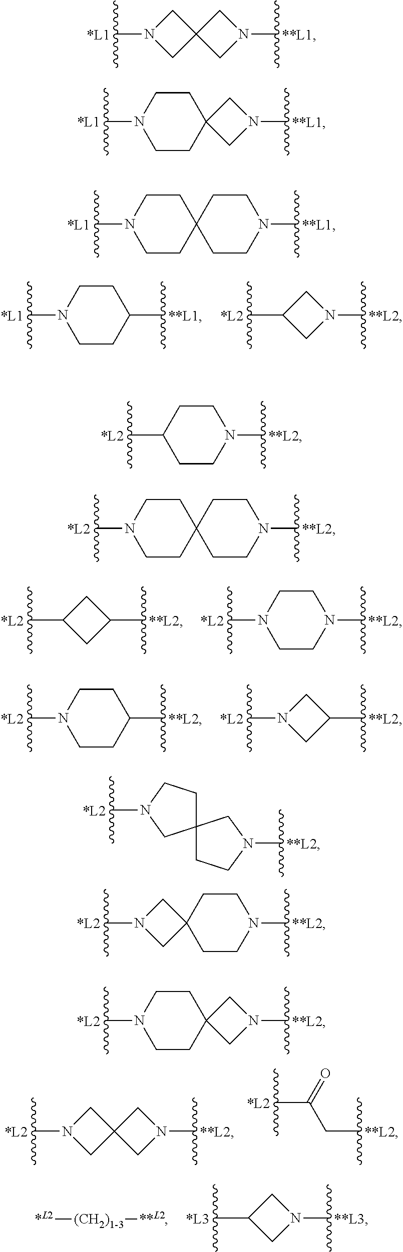

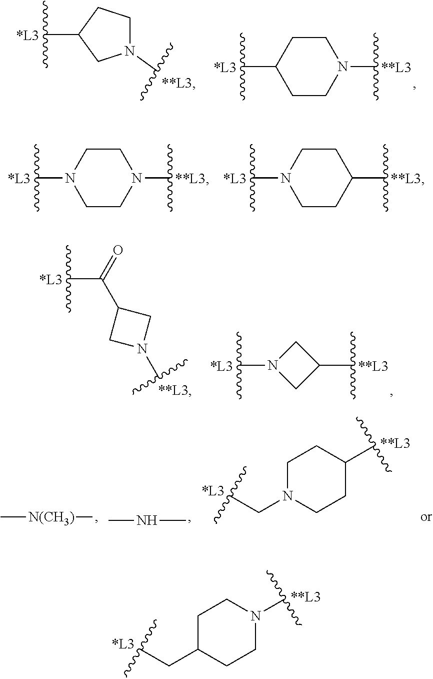

is optionally substituted with 0 to 2 R^{La};
R^{La}, at each occurrence, is independently halogen, —OH, —CN, oxo (═O), —C₁-C₈alkyl, —C₂-C₈alkenyl, —C₂-C₈alkynyl, —C₃-C₈cycloalkyl, 3- to 8-membered heterocyclyl, —C₆-C₁₂aryl, or 5- to 12-membered heteroaryl;
n is 0 or 1.

In another embodiment, the compound is Formula (Ib):

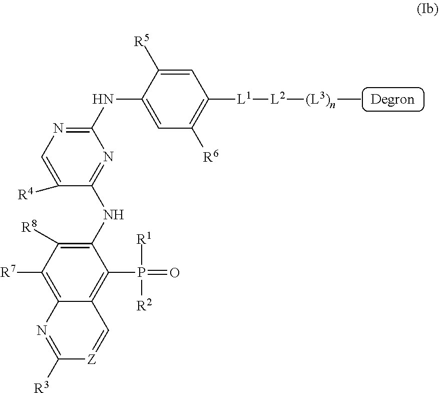

or a N-oxide thereof, or a pharmaceutically acceptable salt thereof, or a stereoisomer thereof, or a deuterated analog thereof, or a prodrug thereof,
wherein:
Z is N or CR^{z};
R^{z} is H, —C₁-C₈alkyl, C₃-C₈cycloalkyl or halogen;
R¹ and R² are each independently —C₁₋₈alkyl or —C₃₋₈cycloalkyl; each of said —C₁-C₈alkyl or C₃-C₈cycloalkyl is optionally substituted with at least one halogen;
R³ is hydrogen, —C₁-C₈alkyl, —C₂-C₈alkenyl, —C₂-C₈alkynyl, C₃-C₈cycloalkyl, halogen, 3- to 8-membered heterocyclyl, C₆-C₁₂aryl, 5- to 12-membered heteroaryl, —OR^{3a}, —SR^{3a}, —CN, —C(O)R^{3a}, —CO₂R^{3a}, —C(O)NR^{3a}R^{3b}, —NR^{3a}R^{3b}, —NR^{3a}COR^{3b}; each of said —C₁-C₈alkyl, —C₂-C₈alkenyl, —C₂-C₈alkynyl, C₃-C₈cycloalkyl, 3- to 8-membered heterocyclyl, C₆-C₁₂aryl or 5- to 12-membered heteroaryl is optionally substituted with 0 to 2 R^{3c};
R^{3a} and R^{3b} are each independently selected from hydrogen, —C₁-C₈alkyl, —C₁-C₈haloalkyl, —C₂-C₈alkenyl, —C₂-C₈alkynyl, C₁-C₈alkoxy-C₁-C₈alkyl-, C₃-C₈cycloalkyl, 3- to 8-membered heterocyclyl, C₆-C₁₂aryl or 5- to 12-membered heteroaryl;
R^{3c}, at each occurrence, is independently halogen, —OH, —CN, oxo (═O), —C₁-C₈alkyl, —C₂-C₈alkenyl, —C₂-C₈alkynyl, —C₃-C₈cycloalkyl, 3- to 8-membered heterocyclyl, —C₆-C₁₂aryl, or 5- to 12-membered heteroaryl;
R⁴ is halogen, methyl or methoxy;
R⁵ is hydrogen, halogen, —C₁-C₈alkyl, —NR^{5a}R^{5b}, —OR^{5a}, —SR^{5a}, C₃-C₈cycloalkyl, 3- to 8-membered heterocyclyl, C₆-C₁₂aryl, 5- to 12-membered heteroaryl, or CN; each of —C₁-C₈alkyl, C₃-C₈cycloalkyl, 3- to 8-membered heterocyclyl, C₆-C₁₂aryl, or 5- to 12-membered heteroaryl is optionally substituted with at least one R^{5c};
R^{5a} and R^{5b} are each independently selected from hydrogen, —C₁-C₈alkyl, —C₂-C₈alkenyl, —C₂-C₈alkynyl, C₃-C₈cycloalkyl, 3- to 8-membered heterocyclyl, C₆-C₁₂aryl, or 5- to 12-membered heteroaryl, each of said —C₁-C₈alkyl, —C₂-C₈alkenyl, —C₂-C₈alkynyl, C₃-C₈cycloalkyl, 3- to 8-membered heterocyclyl, C₆-C₁₂aryl, or 5- to 12-membered heteroaryl is optionally substituted with at least one substituent R^{5d};
R^{5c} and R^{5d} are each independently selected from halogen, hydroxy, —C₁-C₈alkyl, —C₁-C₈alkoxy, —C₂-C₈alkenyl, —C₂-C₈alkynyl, C₃-C₈cycloalkyl, 3- to 8-membered heterocyclyl, C₆-C₁₂aryl, or 5- to 12-membered heteroaryl;
R⁶ is hydrogen, halogen, —C₁-C₈alkyl, —NR^{6a}R^{6b}, —OR^{6a}, —SR^{6a}, C₃-C₈cycloalkyl, 3- to 8-membered heterocyclyl, C₆-C₁₂aryl, 5- to 12-membered heteroaryl, or CN; each of —C₁-C₈alkyl, C₃-C₈cycloalkyl, 3- to 8-membered heterocyclyl, C₆-C₁₂aryl, or 5- to 12-membered heteroaryl is optionally substituted with at least one R^{6c};
R^{6a} and R^{6b} are each independently selected from hydrogen, —C₁-C₈alkyl, —C₂-C₈alkenyl, —C₂-C₈alkynyl, C₃-C₈cycloalkyl, 3- to 8-membered heterocyclyl, C₆-C₁₂aryl, or 5- to 12-membered heteroaryl, each of said —C₁-C₈alkyl, —C₂-C₈alkenyl, —C₂-C₈alkynyl, C₃-C₈cycloalkyl, 3- to 8-membered heterocyclyl, C₆-C₁₂aryl, or 5- to 12-membered heteroaryl is optionally substituted with at least one substituent R^{6d};
R^{6c} and R^{6d} are each independently selected from halogen, hydroxy, —C₁-C₈alkyl, —C₁-C₈alkoxy, —C₂-C₈alkenyl, —C₂-C₈alkynyl, C₃-C₈cycloalkyl, 3- to 8-membered heterocyclyl, C₆-C₁₂aryl, or 5- to 12-membered heteroaryl;
R⁷ and R⁸ are each independently selected from hydrogen, halogen, —C₁-C₈alkyl, —NR^{7a}R^{7b}, —OR^{7a}, —C₃-C₈cycloalkyl, 3- to 8-membered heterocyclyl, —C₆-C₁₂aryl, 5- to 12-membered heteroaryl, or —CN; each of —C₁-C₈alkyl, C₃-C₈cycloalkyl, 3- to 8-membered heterocyclyl, C₆-C₁₂aryl or 5- to 12-membered heteroaryl is optionally substituted with at least one substituent R^{7c}; or
R^{7a} and R^{7b} are each independently selected from hydrogen, —C₁-C₈alkyl, —C₂-C₈alkenyl, —C₂-C₈alkynyl, —C₃-C₈cycloalkyl, 3- to 8-membered heterocyclyl, —C₆-C₁₂aryl or 5- to 12-membered heteroaryl; each of said —C₁-C₈alkyl, —C₂-C₈alkenyl, —C₂-C₈alkynyl, C₃-C₈cycloalkyl, 3- to 8-membered heterocyclyl, C₆-C₁₂aryl or 5- to 12-membered heteroaryl is optionally substituted with at least one substituent R^{7d};
R^{7c} and R^{7d} are each independently halogen, hydroxy, —C₁-C₈alkyl, —C₁-C₈alkoxy, —C₂-C₈alkenyl, —C₂-C₈alkynyl, —C₃-C₈cycloalkyl, 3- to 8-membered heterocyclyl, —C₆-C₁₂aryl or 5- to 12-membered heteroaryl;

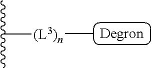

is selected from

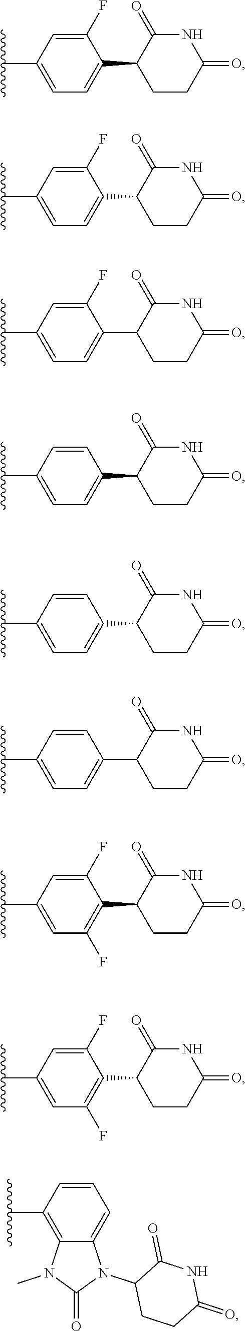

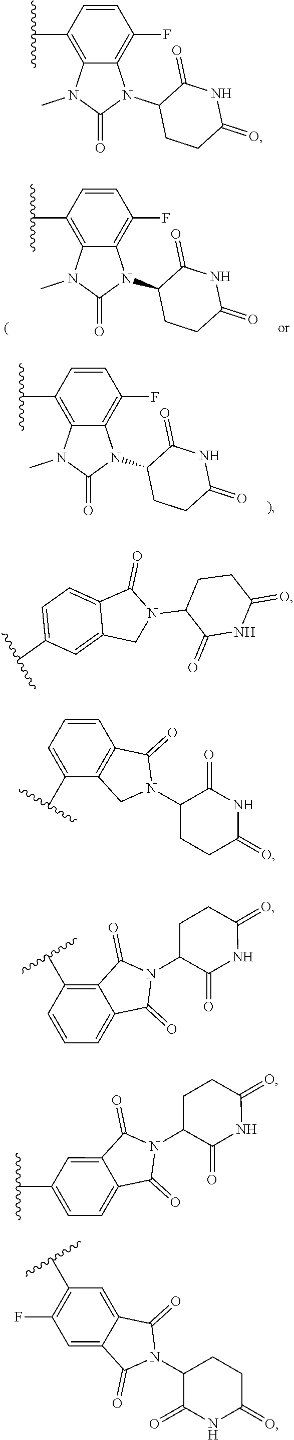

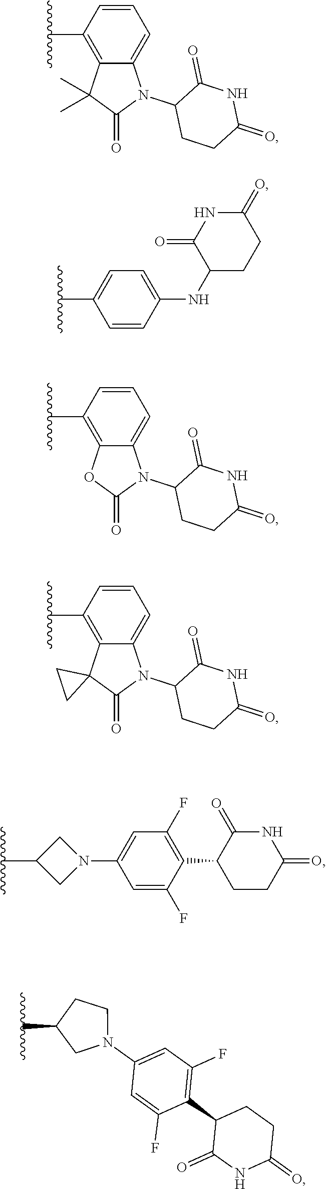 [SMILES: *c1cccc2c1C(C)(C)C(=O)N2C1CCC(=O)NC1=O ; *c1ccc(NC2CCC(=O)NC2=O)cc1 ; *c1cccc2c1oc(=O)n2C1CCC(=O)NC1=O ; *c1cccc2c1C1(CC1)C(=O)N2C1CCC(=O)NC1=O ; *C1CN(c2cc(F)c([C@H]3CCC(=O)NC3=O)c(F)c2)C1 ; *[C@H]1CCN(c2cc(F)c([C@H]3CCC(=O)NC3=O)c(F)c2)C1]

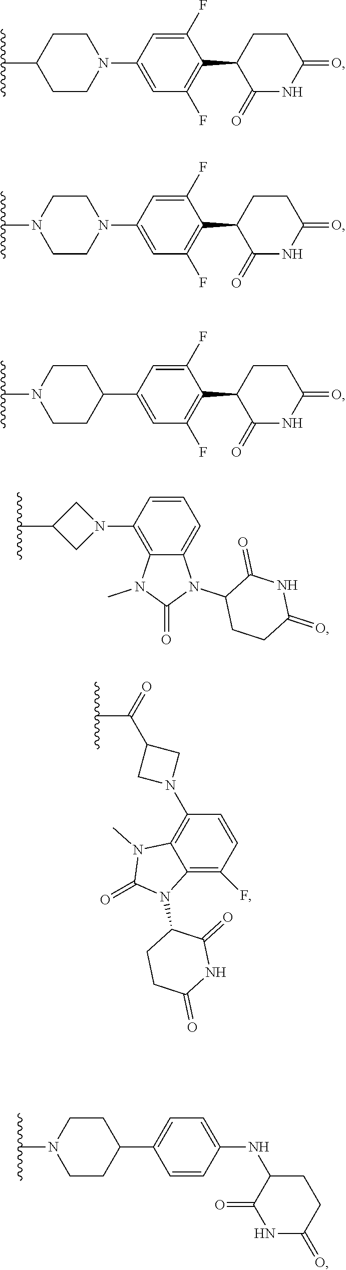

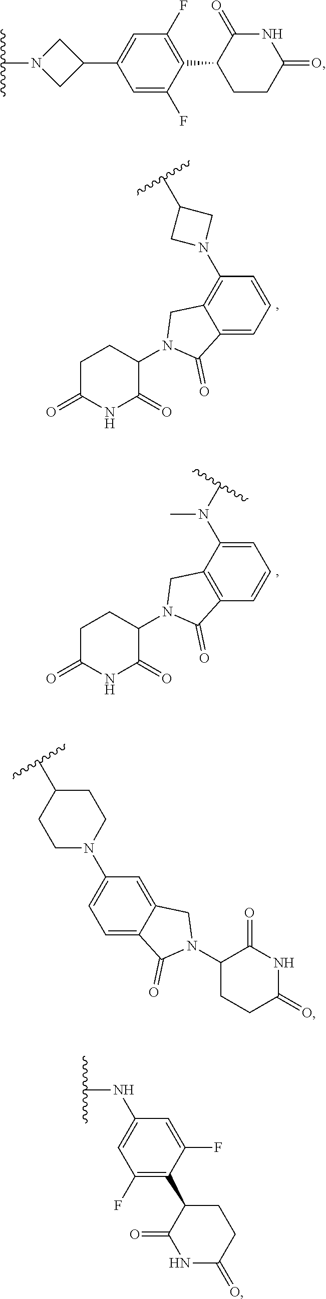 [SMILES: *N1CC(c2cc(F)c([C@H]3CCC(=O)NC3=O)c(F)c2)C1 ; *C1CN(c2cccc3c2CN(C2CCC(=O)NC2=O)C3=O)C1 ; *N(C)c1cccc2c1CN(C1CCC(=O)NC1=O)C2=O ; *C1CCN(c2ccc3c(c2)CN(C2CCC(=O)NC2=O)C3=O)CC1 ; *Nc1cc(F)c([C@H]2CCC(=O)NC2=O)c(F)c1]

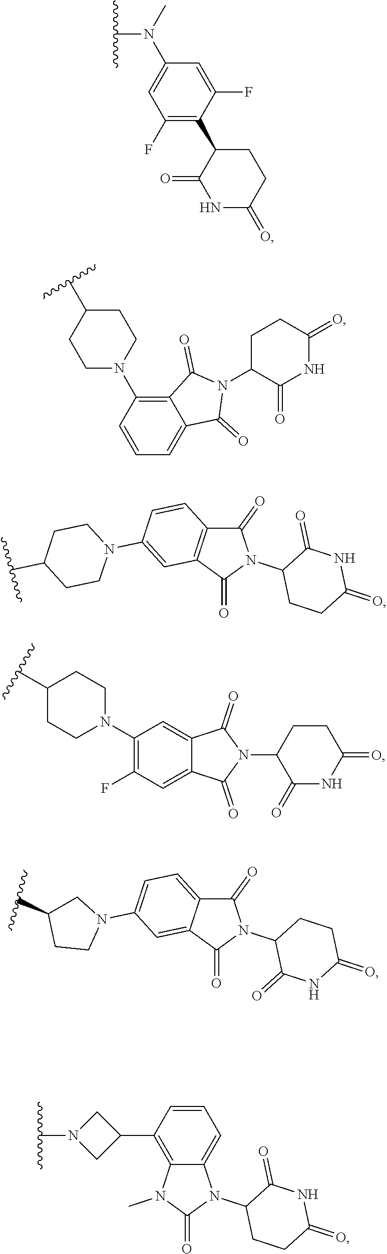 [SMILES: *N(C)c1cc(F)c([C@H]2CCC(=O)NC2=O)c(F)c1 ; *C1CCN(c2cccc3c2C(=O)N(C2CCC(=O)NC2=O)C3=O)CC1 ; *C1CCN(c2ccc3c(c2)C(=O)N(C2CCC(=O)NC2=O)C3=O)CC1 ; *C1CCN(c2cc3c(cc2F)C(=O)N(C2CCC(=O)NC2=O)C3=O)CC1 ; *[C@@H]1CCN(c2ccc3c(c2)C(=O)N(C2CCC(=O)NC2=O)C3=O)C1 ; *N1CC(c2cccc3c2n(C)c(=O)n3C2CCC(=O)NC2=O)C1]

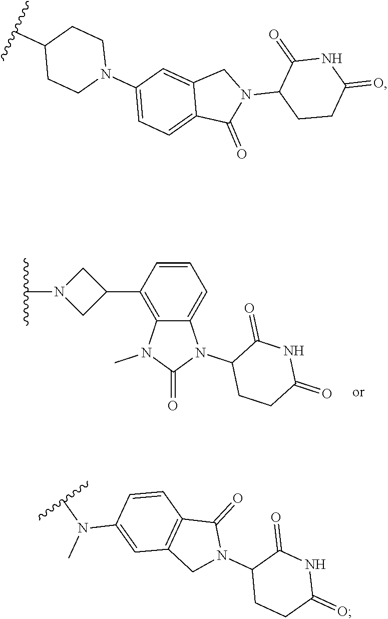 [SMILES: *C1CCN(c2ccc3c(c2)CN(C2CCC(=O)NC2=O)C3=O)CC1 ; *N1CC(c2cccc3c2n(C)c(=O)n3C2CCC(=O)NC2=O)C1 ; *N(C)c1ccc2c(c1)CN(C1CCC(=O)NC1=O)C2=O]

L¹ is

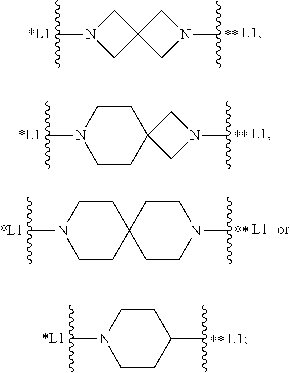

wherein *^{L1} refers to the position attached to the

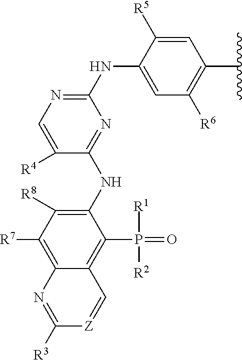

moiety, and **^{L1} refers to the position attached to the

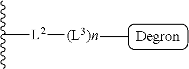

moiety;
L² is

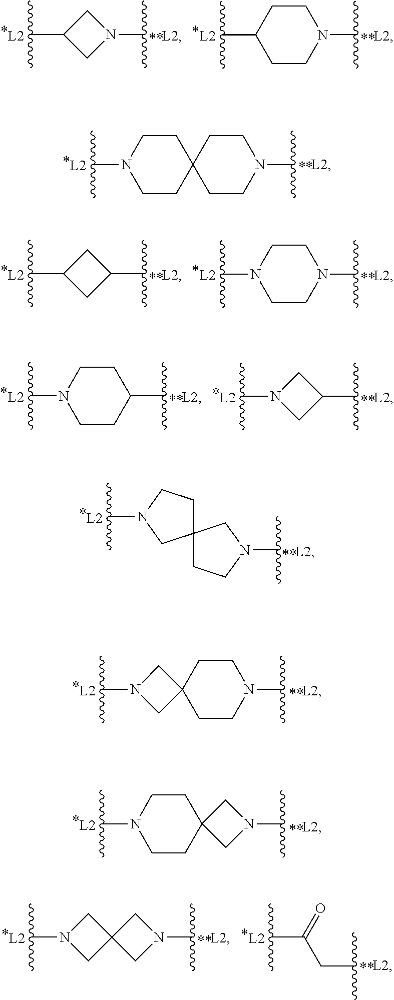

or *^{L2}-(CH₂)₁₋₃—**^{L2}; wherein *^{L2} refers to the position attached to the

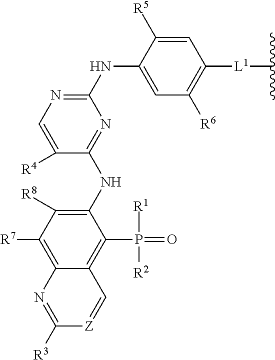

moiety, and **^{L2} refers to the position attached to the

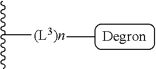

moiety;
each of said

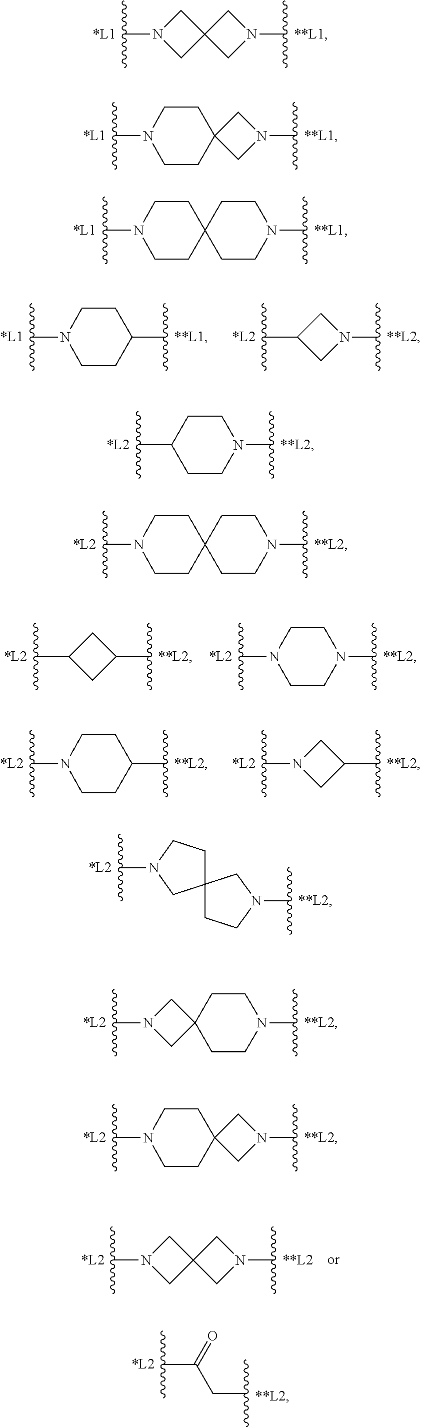

*^{L2}—(CH₂)₁₋₃—**^{L2} is optionally substituted with 0 to 2 R^{La};
R^{La} at each occurrence, is independently halogen, —OH, —CN, oxo (═O), —C₁-C₈alkyl, —C₂-C₈alkenyl, —C₂-C₈alkynyl, —C₃-C₈cycloalkyl, 3- to 8-membered heterocyclyl, —C₆-C₁₂aryl, or 5- to 12-membered heteroaryl;
n is 0 or 1;

Aspect 2. The compound of Aspect 1, wherein the compound is formula (IIa) or (IIb)

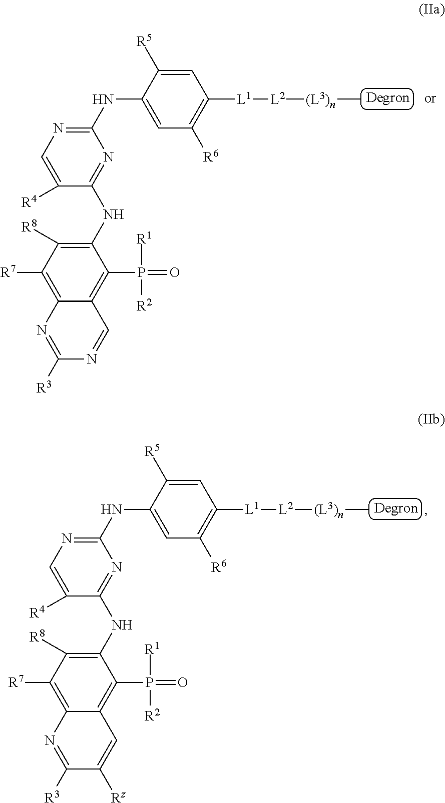

wherein
R^{z}, R¹, R², R³, R⁴, R⁵, R⁶, R⁷, R⁸,

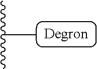

L¹, L², L³ and n are each defined as Aspect 1.

Aspect 3. The compound of Aspect 1, wherein the compound is formula (IIIa) to (IIIu)

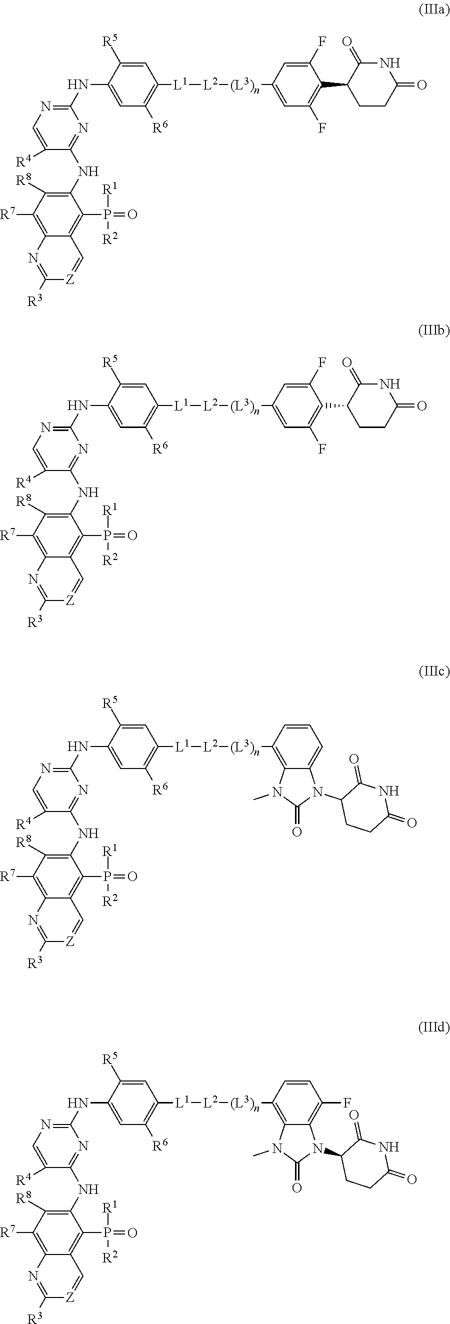

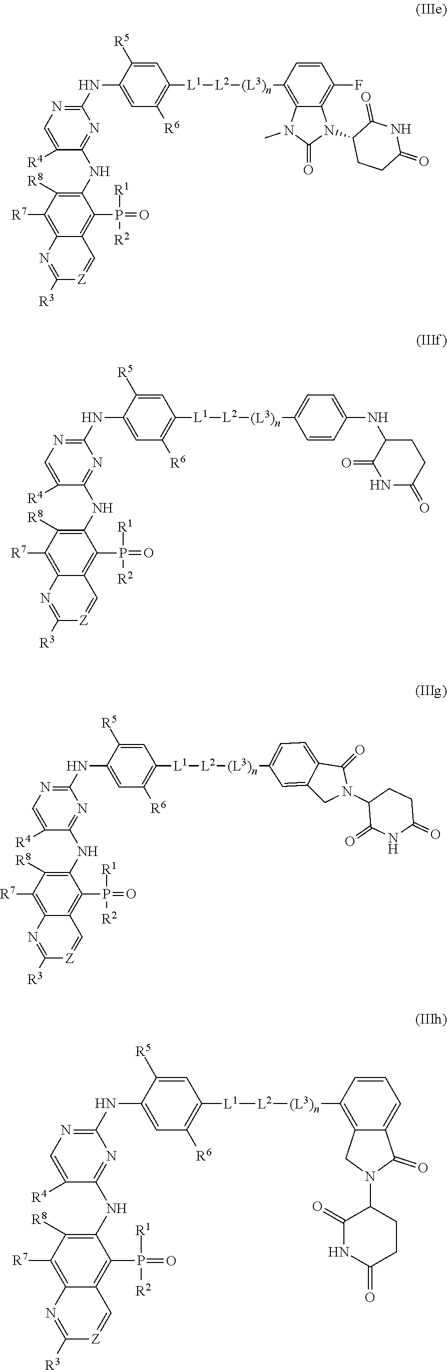

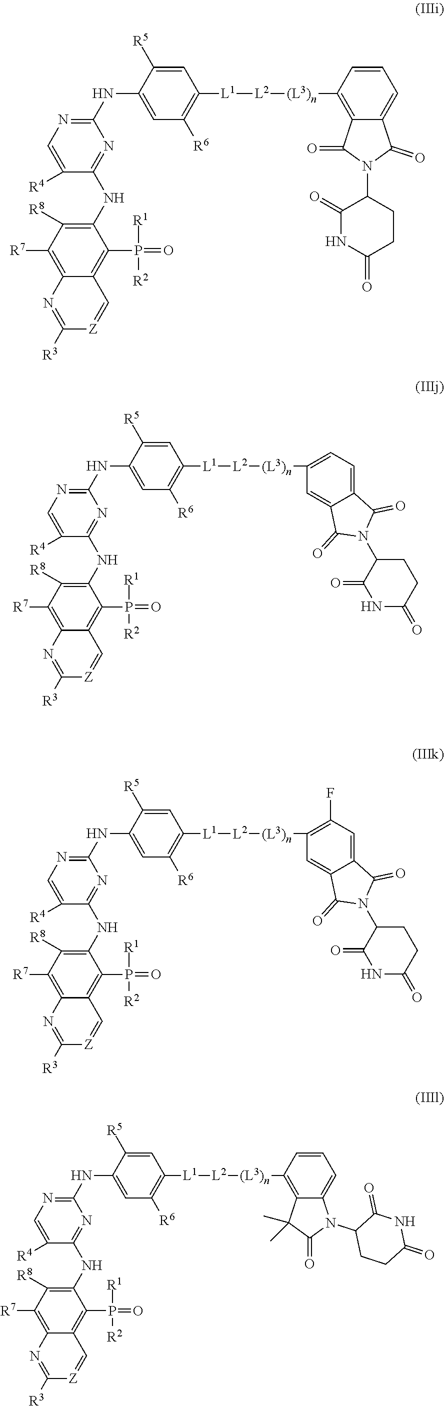

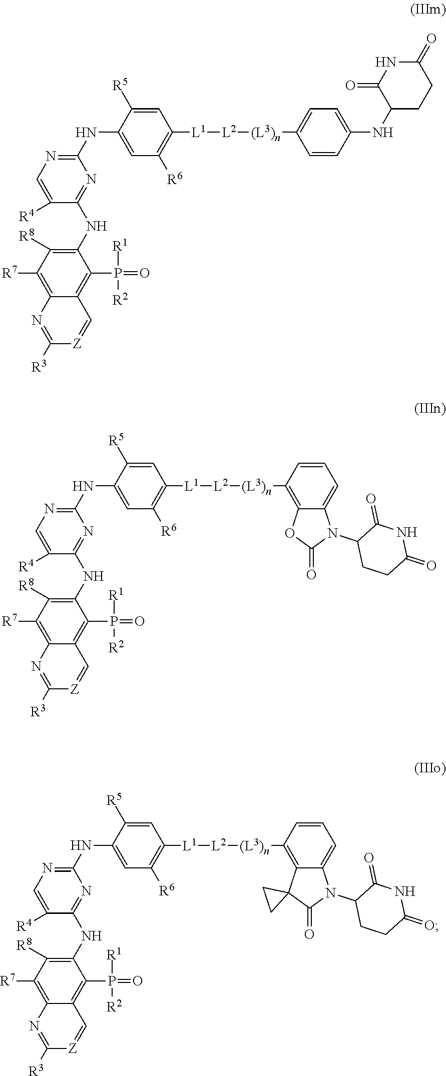

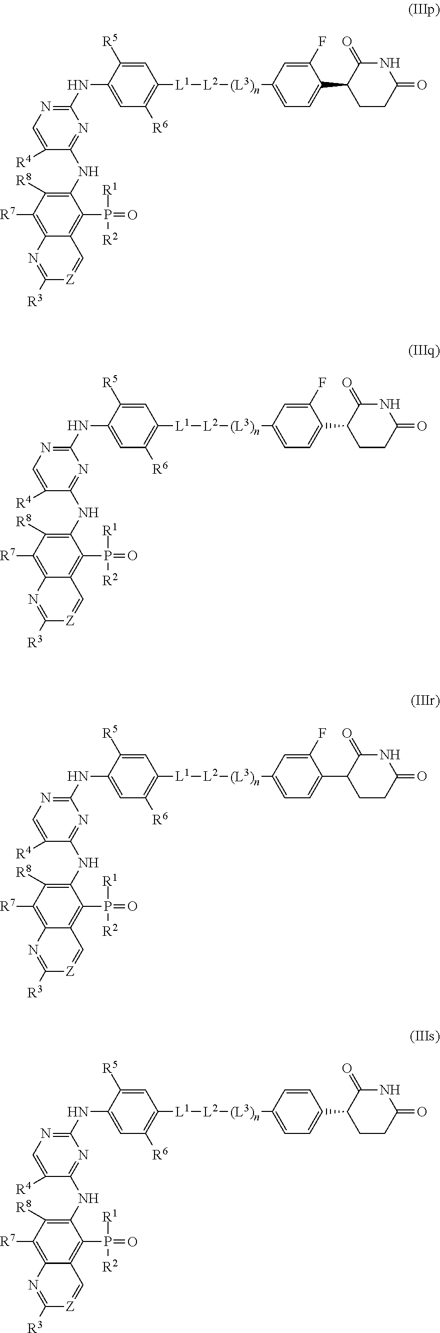

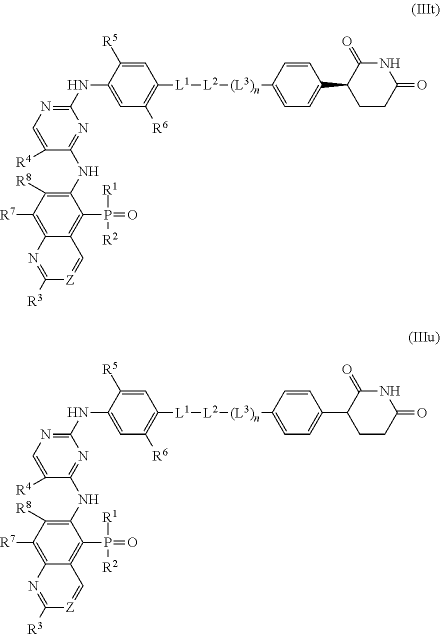

wherein
R¹, R², R³, R⁴, R⁵, R⁶, R⁷, R⁸, Z, L¹, L², L³ and n are each defined as Aspect 1.

Aspect 4. The compound of Aspect 1, wherein the compound is formula (IVa) to (IVh)

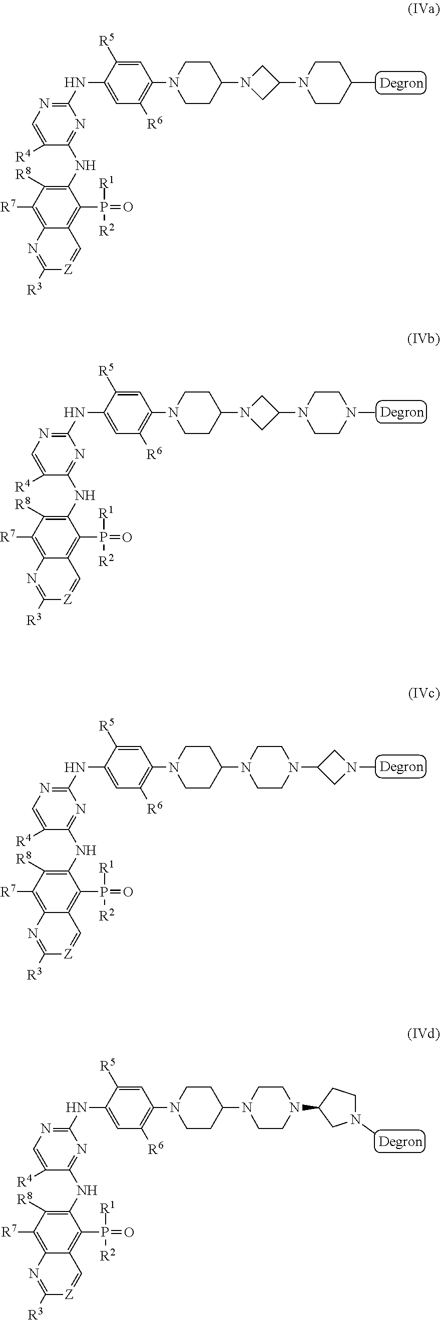

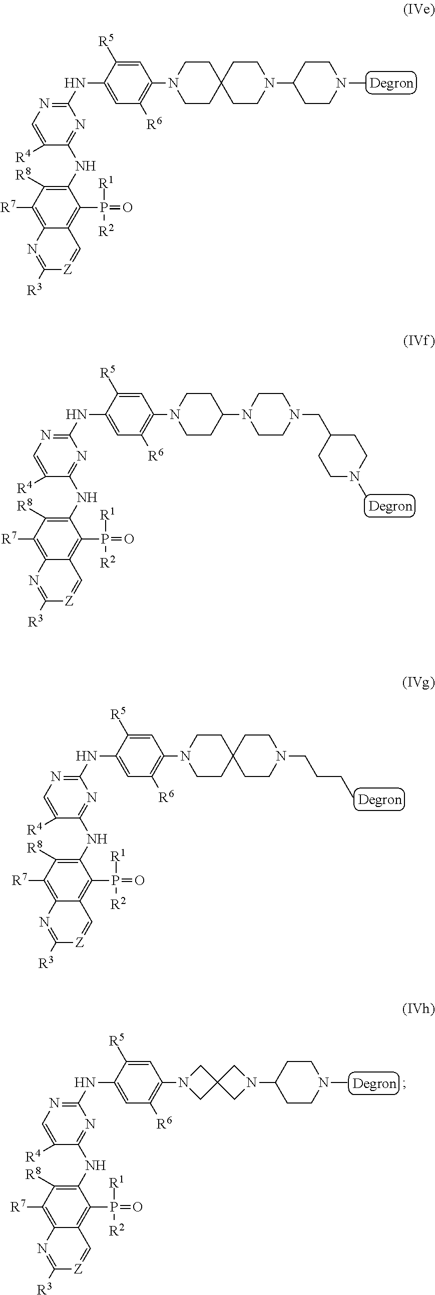

wherein
Z, R¹, R², R³, R⁴, R⁵, R⁶, R⁷, R⁸ and

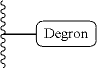

are each defined as Aspect 1.

Aspect 5. The compound of any preceding Aspects, wherein Z is N or CR^{z};
R^{z} is H, methyl, ethyl, propyl, butyl, pentyl, hexyl, heptyl, octyl, cyclopropyl, cyclobutyl, cyclopentyl, cyclohexyl, cycloheptyl, cyclooctyl, —F, —Cl, —Br or —I.

Aspect 6. The compound of any preceding Aspects, wherein Z is N or CR^{z};
R^{z} is H, methyl, ethyl, n-propyl, iso-propyl, n-butyl, iso-butyl, sec-butyl, tert-butyl, pentyl, cyclopropyl, cyclobutyl, cyclopentyl, cyclohexyl, —F, —Cl, —Br or —I.

Aspect 7. The compound of any preceding Aspects, wherein R¹ and R² are each independently methyl, ethyl, propyl, butyl, pentyl, hexyl, heptyl, octyl, cyclopropyl, cyclobutyl, cyclopentyl, cyclohexyl, cycloheptyl or cyclooctyl.

Aspect 8. The compound of any preceding Aspects, wherein R¹ and R² are each independently methyl.

Aspect 9. The compound of any preceding Aspects, wherein R³ is methyl, ethyl, propyl, butyl, pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, —F, —Cl, —Br, —I, 3- to 8-membered heterocyclyl, phenyl, 5- to 12-membered heteroaryl, —OR^{3a}, —SR^{3a}, —CN, —C(O)R^{3a}, —CO₂R^{3a}, —C(O)NR^{3a}R^{3b}, —NR^{3a}R^{3b}, —NR^{3a}COR^{3b}; each of said methyl, ethyl, propyl, butyl, pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, —F, —Cl, —Br, —I, 3- to 8-membered heterocyclyl, phenyl or 5- to 12-membered heteroaryl is optionally substituted with 0, 1 or 2 R^{3c};
R^{3a} and R^{3b} are each independently selected from hydrogen, methyl, ethyl, propyl, butyl, pentyl, hexyl, heptyl, octyl, —C₁-C₈haloalkyl, —C₂-C₈alkenyl, —C₂-C₈alkynyl, C₁-C₈alkoxy-C₁-C₈alkyl-, cyclopropyl, cyclobutyl, cyclopentyl, cyclohexyl, cycloheptyl, cyclooctyl, 3- to 8-membered heterocyclyl, phenyl or 5- to 12-membered heteroaryl;
R^{3c}, at each occurrence, is independently —F, —Cl, —Br, —I, —OH, —CN, oxo (═O), methyl, ethyl, propyl, butyl, pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl.

Aspect 10. The compound of any preceding Aspects, wherein R³ is methyl, ethyl, propyl, butyl, pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, —F, —Cl, —Br, —I, 3- to 8-membered heterocyclyl, phenyl, 5- to 12-membered heteroaryl;
preferably, R³ is methyl, ethyl, propyl (n-propyl, iso-propyl), butyl (n-butyl, iso-butyl, sec-butyl, tert-butyl), pentyl, hexyl, heptyl, octyl.

Aspect 11. The compound of any preceding Aspects, wherein R⁴ is —F, —Cl, —Br, —I, Me, OMe.

Aspect 12. The compound of any preceding Aspects, wherein R⁴ is —Cl or —Br.

Aspect 13. The compound of any preceding Aspects, wherein R⁵ is hydrogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, phenyl, 5- to 12-membered heteroaryl, —NR^{5a}R^{5b}, —OR^{5a}, —SR^{5a}, or CN; each of said methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one R^{5c};
R^{5a} and R^{5b} are each independently selected from hydrogen, methyl, ethyl, propyl, butyl, pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl, each of said methyl, ethyl, propyl, butyl, pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl is optionally substituted with at least one substituent R^{5d};
R^{5c} and R^{5d} are each independently selected from —F, —Cl, —Br, —I, hydroxy, methyl, ethyl, propyl, butyl, pentyl, hexyl, heptyl, octyl, methoxy, ethoxy, propoxy, butoxy, pentoxy, hexoxy, heptoxy, octoxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl.

Aspect 14. The compound of any preceding Aspects, wherein R⁵ is —OR^{5a};
R^{5a} is selected from hydrogen, methyl, ethyl, propyl, butyl, pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl, each of said methyl, ethyl, propyl, butyl, pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl is optionally substituted with at least one substituent R^{5d};
R^{5d} is independently selected from —F, —Cl, —Br, —I, hydroxy, methyl, ethyl, propyl, butyl, pentyl, hexyl, heptyl, octyl, methoxy, ethoxy, propoxy, butoxy, pentoxy, hexoxy, heptoxy, octoxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl;
preferably, R⁵ is methoxy, ethoxy, propoxy (n-propoxy, iso-propoxy), butoxy (n-butoxy, iso-butoxy, sec-butoxy, tert-butoxy), pentoxy, hexoxy, heptoxy or octoxy.

Aspect 15. The compound of any preceding Aspects, wherein R⁶ is hydrogen, —F, —Cl, —Br, —I, methyl, ethyl, propyl, butyl, pentyl, hexyl, heptyl, octyl, —NR^{6a}R^{6b}, —OR^{6a}, —SR^{6a}, cyclopropyl, cyclobutyl, cyclopentyl, cyclohexyl, cycloheptyl, cyclooctyl, 3- to 8-membered heterocyclyl, phenyl, 5- to 12-membered heteroaryl, or CN; each of said methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one R^{6c};
R^{6a} and R^{6b} are each independently selected from hydrogen, methyl, ethyl, propyl, butyl, pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl, each of said methyl, ethyl, propyl, butyl, pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent R^{6d};
R^{6c} and R^{6d} are each independently selected from —F, —Cl, —Br, —I, methyl, ethyl, propyl, butyl, pentyl, hexyl, heptyl, octyl, methoxy, ethoxy, propoxy, butoxy, pentoxy, hexoxy, heptoxy, octoxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl.

Aspect 16. The compound of any preceding Aspects, wherein R⁶ is hydrogen, methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, phenyl, 5- to 12-membered heteroaryl, or CN; each of said methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one R^{6c};
R^{6c} is independently selected from —F, —Cl, —Br, —I, methyl, ethyl, propyl, butyl, pentyl, hexyl, heptyl, octyl, methoxy, ethoxy, propoxy, butoxy, pentoxy, hexoxy, heptoxy, octoxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl;
preferably, R⁶ is hydrogen, methyl, ethyl, propyl (n-propyl, iso-propyl), butyl (n-butyl, sec-butyl, iso-butyl, tert-butyl), pentyl, hexyl, heptyl, octyl, cyclopropyl, cyclobutyl, cyclopentyl, cyclohexyl, cycloheptyl, cyclooctyl.

Aspect 17. The compound of any preceding Aspects, wherein R⁷ and R⁸ are each independently selected from hydrogen, —F, —Cl, —Br, —I, methyl, ethyl, propyl, butyl, pentyl, hexyl, heptyl, octyl, —NR^{7a}R^{7b}, —OR^{7a}, cyclopropyl, cyclobutyl, cyclopentyl, cyclohexyl, cycloheptyl, cyclooctyl, 3- to 8-membered heterocyclyl, phenyl, 5- to 12-membered heteroaryl or —CN; each of said methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent R^{7c}; or
R^{7a} and R^{7b} are each independently selected from hydrogen, methyl, ethyl, propyl, butyl, pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl, each of said methyl, ethyl, propyl, butyl, pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent R^{7d}; or
R^{7c} and R^{7d} are each independently —F, —Cl, —Br, —I, hydroxy, methyl, ethyl, propyl, butyl, pentyl, hexyl, heptyl, octyl, methoxy, ethoxy, propoxy, butoxy, pentoxy, hexoxy, heptoxy, octoxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl.

Aspect 18. The compound of any preceding Aspects, wherein R⁷ and R⁸ are each independently selected from hydrogen, —F, —Cl, —Br, —I, methyl, ethyl, propyl, butyl, pentyl, hexyl, heptyl, octyl, methoxy, ethoxy, propoxy, butoxy, pentoxy, hexoxy, heptoxy, octoxy, cyclopropyl, cyclobutyl, cyclopentyl, cyclohexyl, cycloheptyl, cyclooctyl, 3- to 8-membered heterocyclyl, phenyl or 5- to 12-membered heteroaryl;
preferably, R and R⁸ are each hydrogen, —F, —Cl, —Br or —I.

Aspect 19. The compound of any preceding Aspects, wherein the

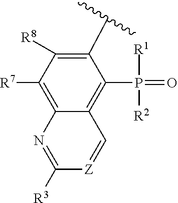

moiety is selected from:

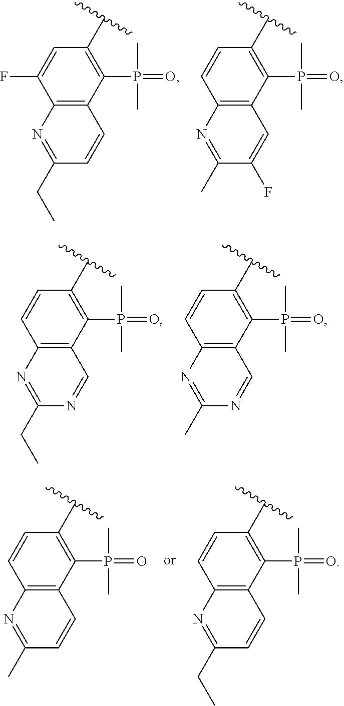

Aspect 20. The compound of any preceding Aspects, wherein the

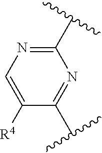

moiety is selected from:

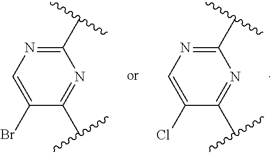 [SMILES: *c1ncc(Br)c(*)n1 ; "or" ; *c1ncc(Cl)c(*)n1 ; "."]

Aspect 21. The compound of any preceding Aspects, wherein the

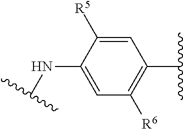

moiety is selected from:

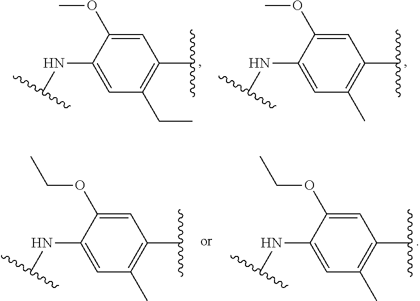 [SMILES: *Nc1cc(CC)c(*)cc1OC ; *Nc1cc(C)c(*)cc1OC ; *Nc1cc(C)c(*)cc1OCC ; *Nc1cc(C)c(*)cc1OCC]

Aspect 22. The compound of any preceding Aspects, wherein the

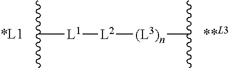

moiety is selected from:

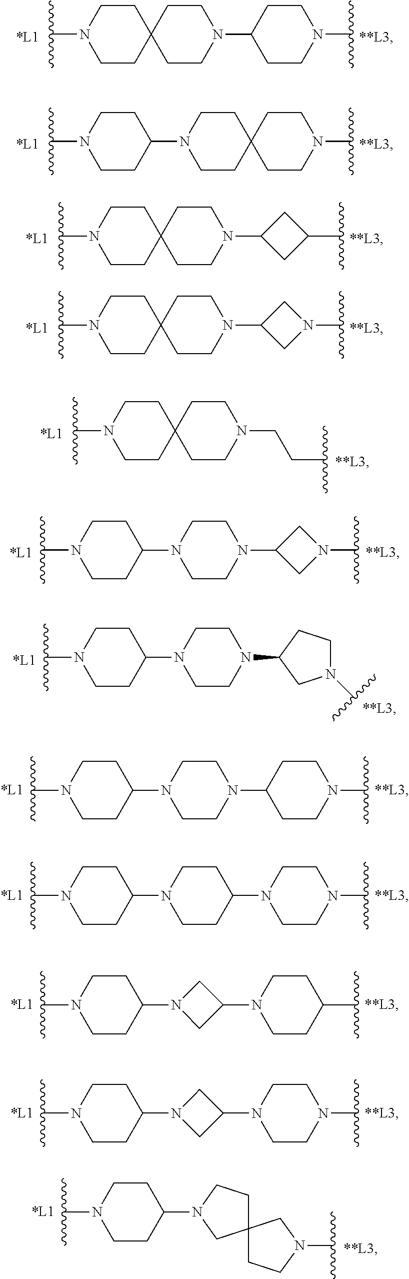

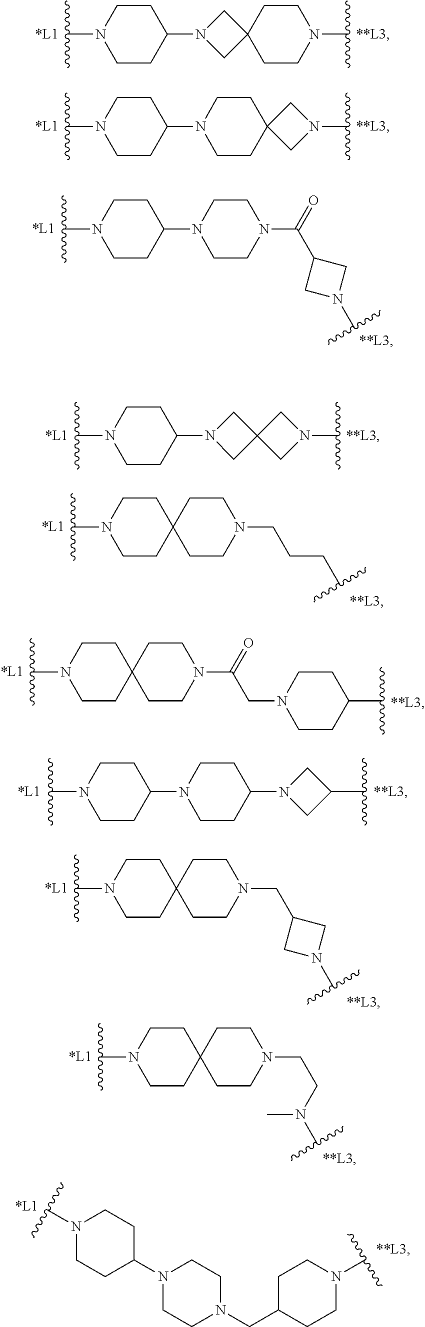

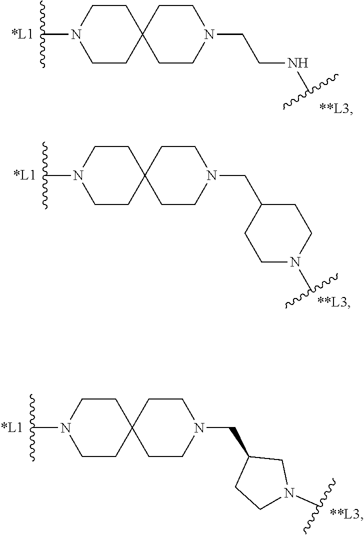 [SMILES: *NCCN1CCC2(CCN(*)CC2)CC1 ; *N1CCC(CN2CCC3(CCN(*)CC3)CC2)CC1 ; *N1CCC2(CC1)CCN(C[C@@H]1CCN(*)C1)CC2]

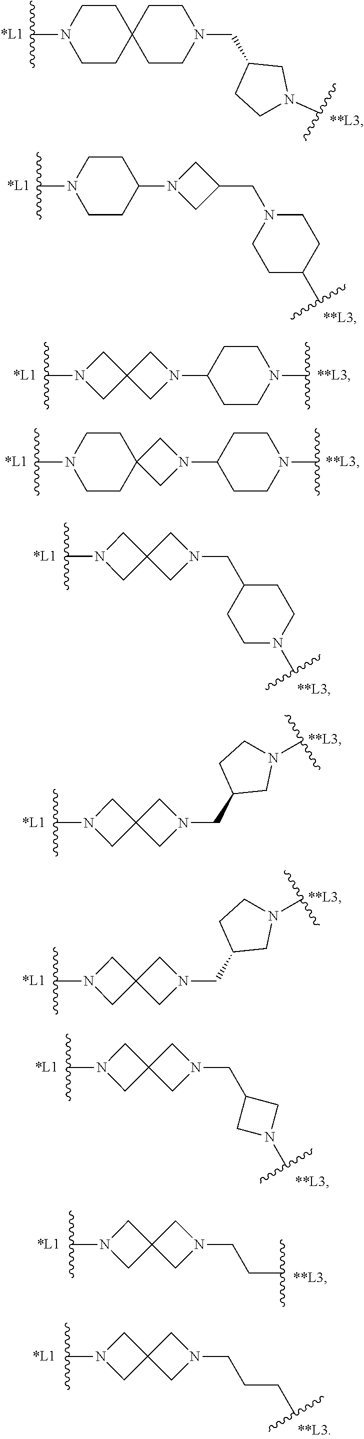

Aspect 23 The compound of any preceding Aspects, wherein the compound is selected from

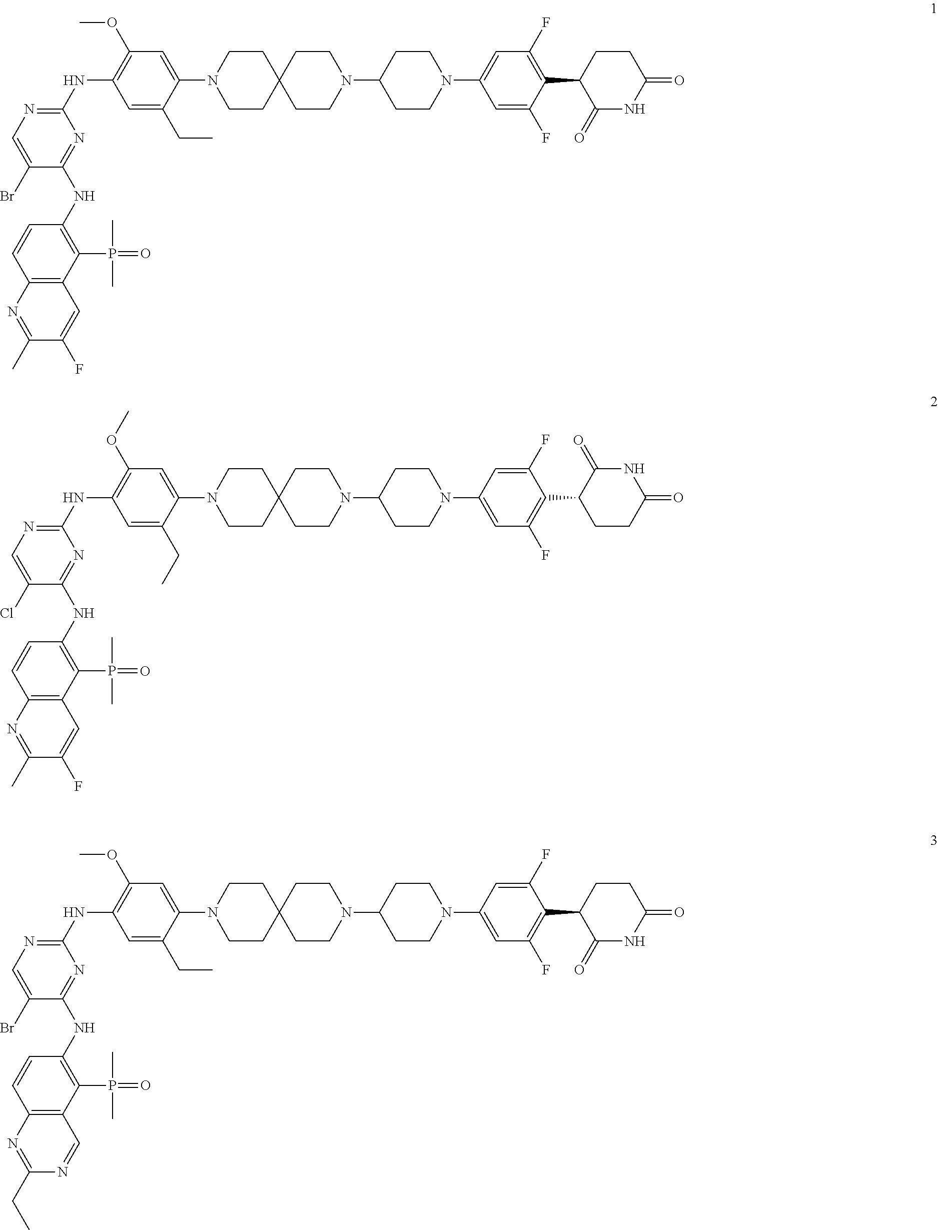 [SMILES: CCc1cc(Nc2ncc(Br)c(Nc3ccc4nc(C)c(F)cc4c3P(C)(C)=O)n2)c(OC)cc1N1CCC2(CC1)CCN(C1CCN(c3cc(F)c([C@H]4CCC(=O)NC4=O)c(F)c3)CC1)CC2 ; CCc1cc(Nc2ncc(Cl)c(Nc3ccc4nc(C)c(F)cc4c3P(C)(C)=O)n2)c(OC)cc1N1CCC2(CC1)CCN(C1CCN(c3cc(F)c([C@H]4CCC(=O)NC4=O)c(F)c3)CC1)CC2 ; CCc1ncc2c(P(C)(C)=O)c(Nc3nc(Nc4cc(CC)c(N5CCC6(CC5)CCN(C5CCN(c7cc(F)c([C@H]8CCC(=O)NC8=O)c(F)c7)CC5)CC6)cc4OC)ncc3Br)ccc2n1]

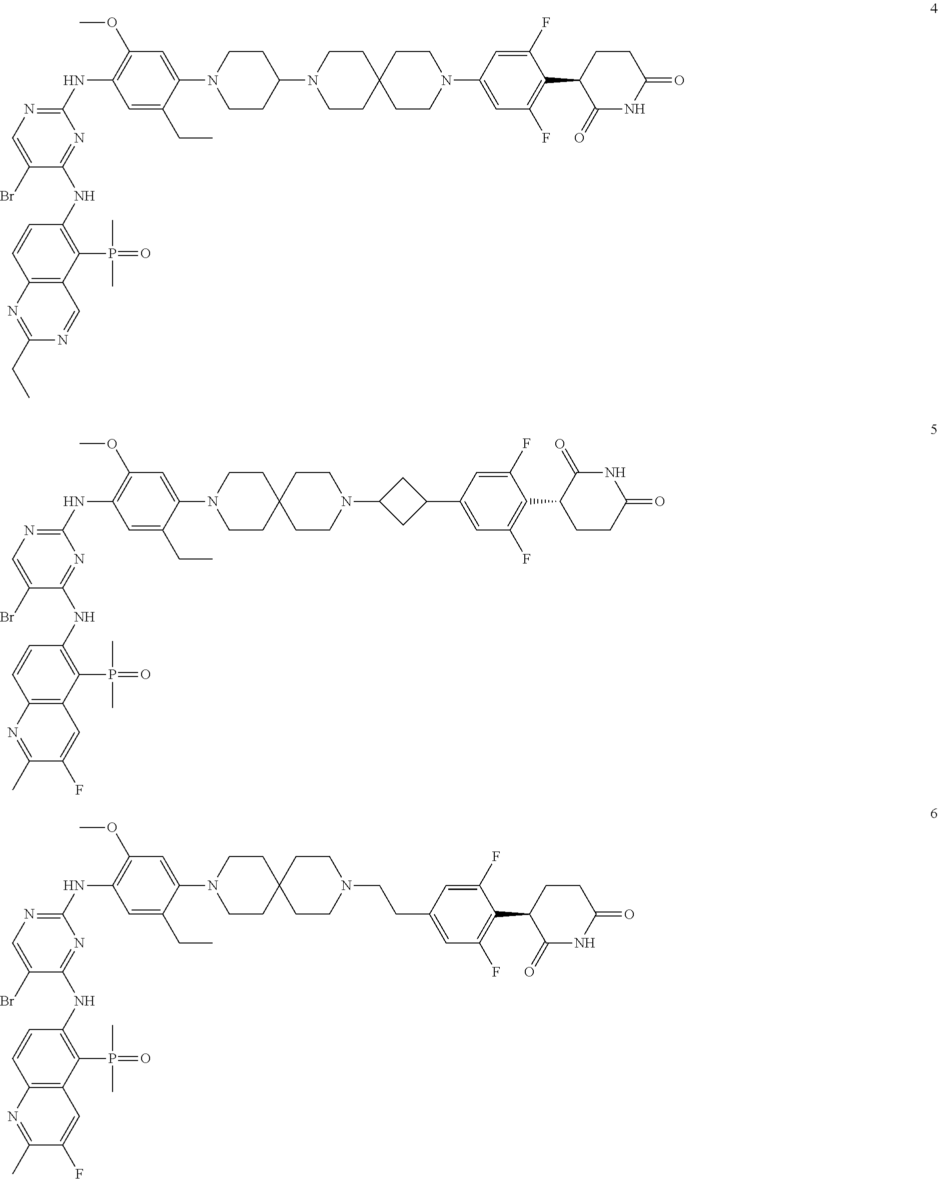

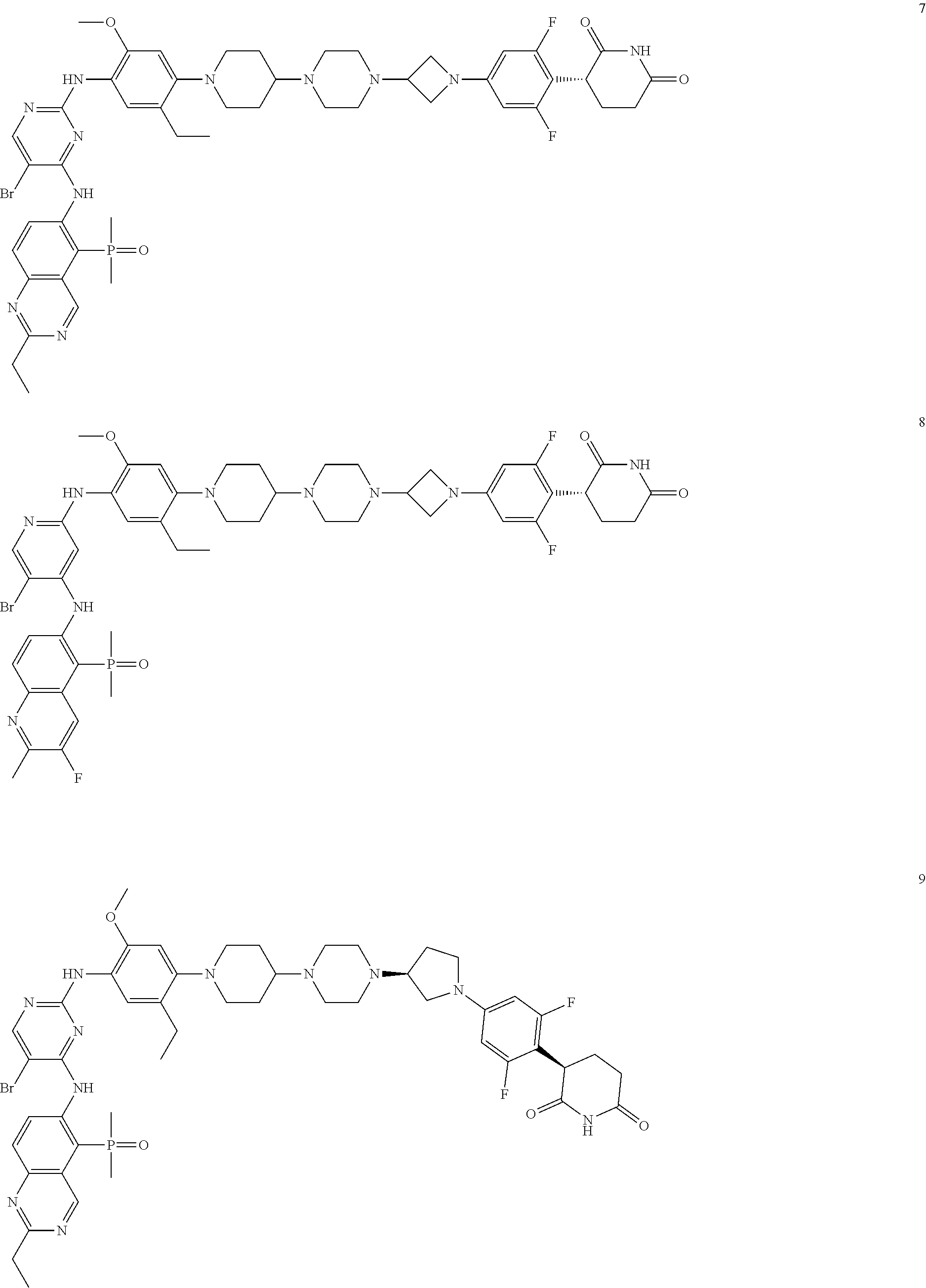 [SMILES: CCc1ncc2c(P(C)(C)=O)c(Nc3nc(Nc4cc(CC)c(N5CCC(N6CCN(C7CN(c8cc(F)c([C@H]9CCC(=O)NC9=O)c(F)c8)C7)CC6)CC5)cc4OC)ncc3Br)ccc2n1 ; CCc1cc(Nc2cc(Nc3ccc4nc(C)c(F)cc4c3P(C)(C)=O)c(Br)cn2)c(OC)cc1N1CCC(N2CCN(C3CN(c4cc(F)c([C@H]5CCC(=O)NC5=O)c(F)c4)C3)CC2)CC1 ; CCc1ncc2c(P(C)(C)=O)c(Nc3nc(Nc4cc(CC)c(N5CCC(N6CCN([C@H]7CCN(c8cc(F)c([C@H]9CCC(=O)NC9=O)c(F)c8)C7)CC6)CC5)cc4OC)ncc3Br)ccc2n1]

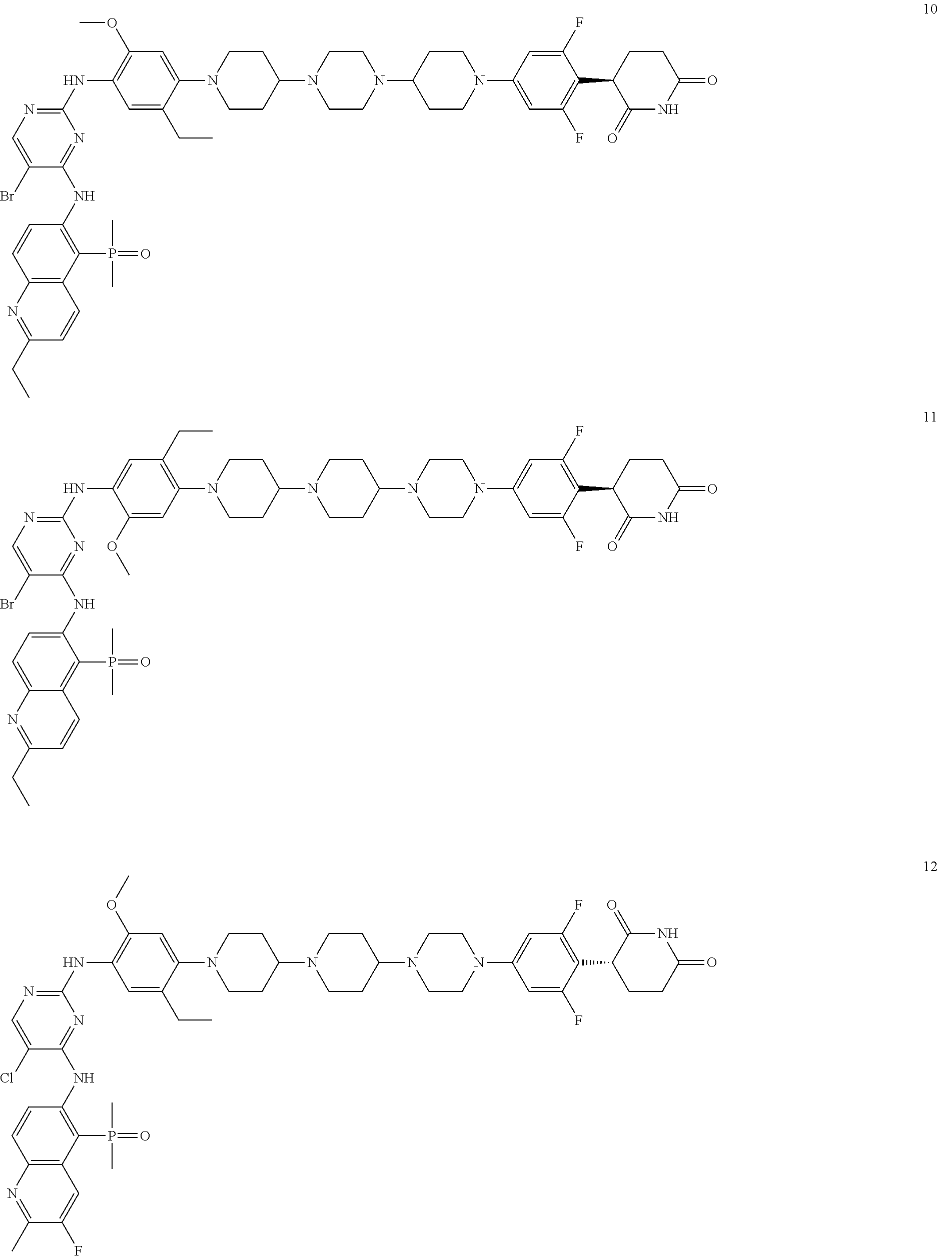 [SMILES: CCc1ccc2c(P(C)(C)=O)c(Nc3nc(Nc4cc(CC)c(N5CCC(N6CCN(C7CCN(c8cc(F)c([C@H]9CCC(=O)NC9=O)c(F)c8)CC7)CC6)CC5)cc4OC)ncc3Br)ccc2n1 ; CCc1ccc2c(P(C)(C)=O)c(Nc3nc(Nc4cc(CC)c(N5CCC(N6CCC(N7CCN(c8cc(F)c([C@H]9CCC(=O)NC9=O)c(F)c8)CC7)CC6)CC5)cc4OC)ncc3Br)ccc2n1 ; CCc1cc(Nc2ncc(Cl)c(Nc3ccc4nc(C)c(F)cc4c3P(C)(C)=O)n2)c(OC)cc1N1CCC(N2CCC(N3CCN(c4cc(F)c([C@H]5CCC(=O)NC5=O)c(F)c4)CC3)CC2)CC1]

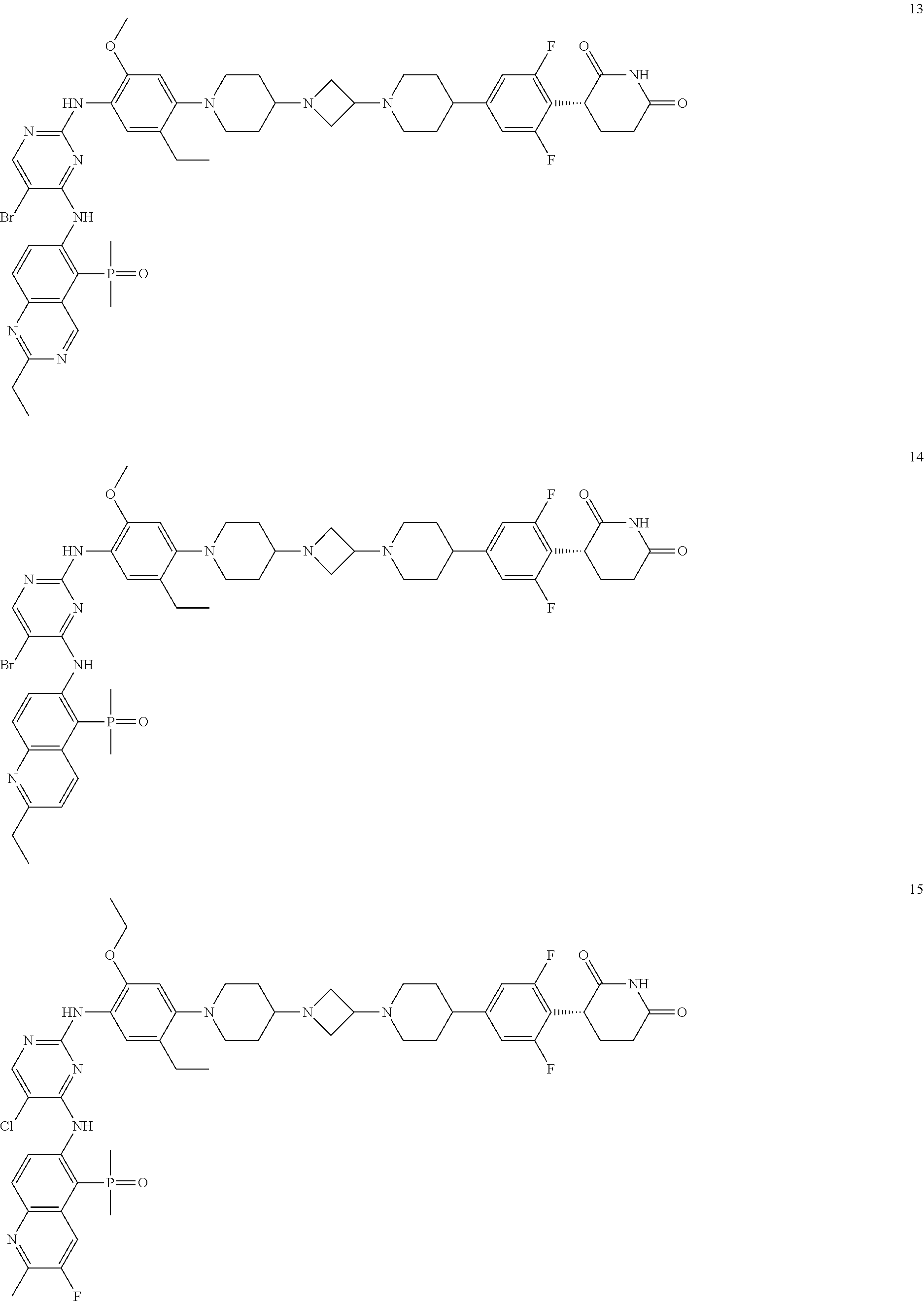 [SMILES: CCc1ncc2c(P(C)(C)=O)c(Nc3nc(Nc4cc(CC)c(N5CCC(N6CC(N7CCC(c8cc(F)c([C@H]9CCC(=O)NC9=O)c(F)c8)CC7)C6)CC5)cc4OC)ncc3Br)ccc2n1 ; CCc1ccc2c(P(C)(C)=O)c(Nc3nc(Nc4cc(CC)c(N5CCC(N6CC(N7CCC(c8cc(F)c([C@H]9CCC(=O)NC9=O)c(F)c8)CC7)C6)CC5)cc4OC)ncc3Br)ccc2n1 ; CCOc1cc(N2CCC(N3CC(N4CCC(c5cc(F)c([C@H]6CCC(=O)NC6=O)c(F)c5)CC4)C3)CC2)c(CC)cc1Nc1ncc(Cl)c(Nc2ccc3nc(C)c(F)cc3c2P(C)(C)=O)n1]

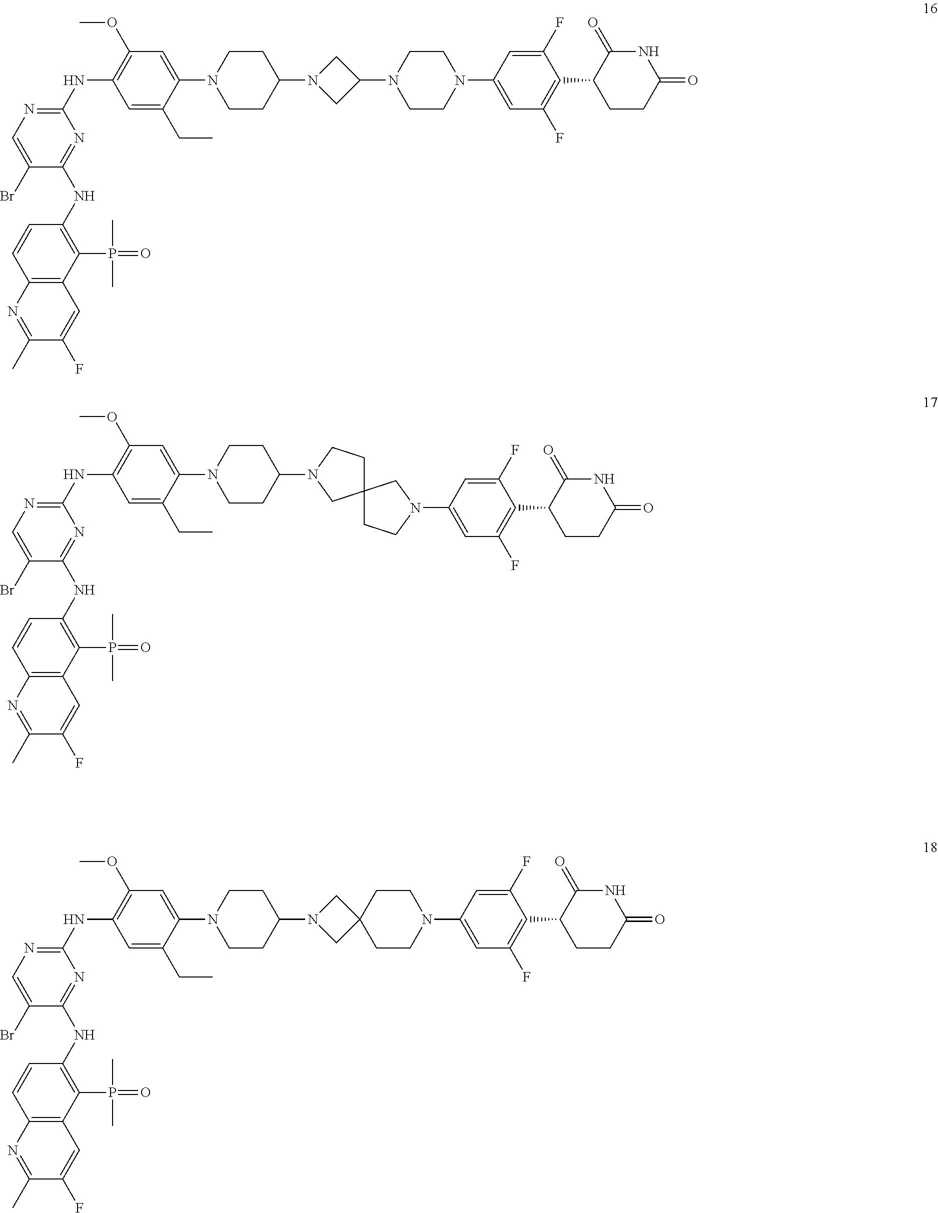 [SMILES: CCc1cc(Nc2ncc(Br)c(Nc3ccc4nc(C)c(F)cc4c3P(C)(C)=O)n2)c(OC)cc1N1CCC(N2CC(N3CCN(c4cc(F)c([C@H]5CCC(=O)NC5=O)c(F)c4)CC3)C2)CC1 ; CCc1cc(Nc2ncc(Br)c(Nc3ccc4nc(C)c(F)cc4c3P(C)(C)=O)n2)c(OC)cc1N1CCC(N2CCC3(CCN(c4cc(F)c([C@H]5CCC(=O)NC5=O)c(F)c4)C3)C2)CC1 ; CCc1cc(Nc2ncc(Br)c(Nc3ccc4nc(C)c(F)cc4c3P(C)(C)=O)n2)c(OC)cc1N1CCC(N2CC3(CCN(c4cc(F)c([C@H]5CCC(=O)NC5=O)c(F)c4)CC3)C2)CC1]

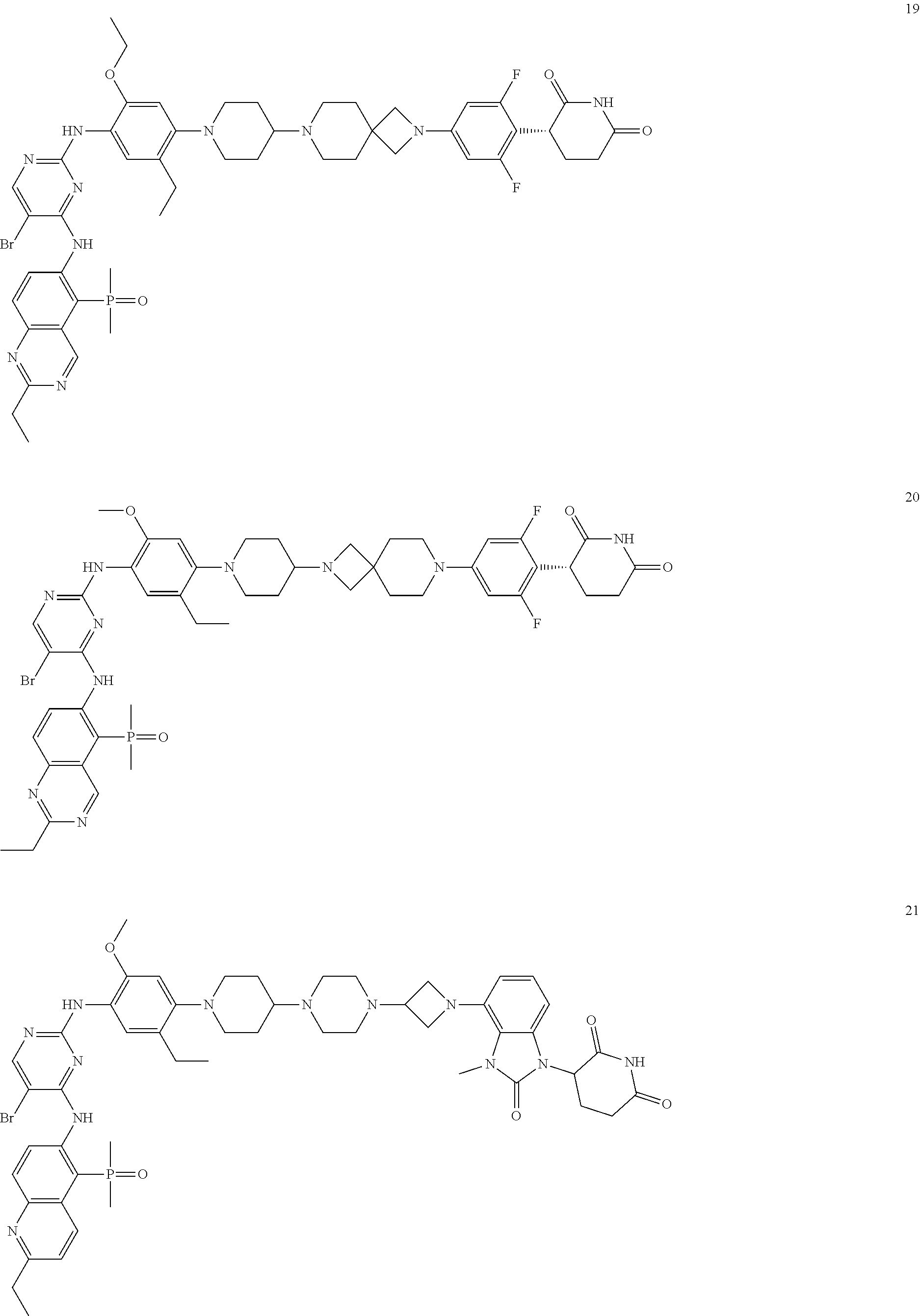 [SMILES: CCOc1cc(N2CCC(N3CCC4(CC3)CN(c3cc(F)c([C@H]5CCC(=O)NC5=O)c(F)c3)C4)CC2)c(CC)cc1Nc1ncc(Br)c(Nc2ccc3nc(CC)ncc3c2P(C)(C)=O)n1 ; CCc1ncc2c(P(C)(C)=O)c(Nc3nc(Nc4cc(CC)c(N5CCC(N6CC7(CCN(c8cc(F)c([C@H]9CCC(=O)NC9=O)c(F)c8)CC7)C6)CC5)cc4OC)ncc3Br)ccc2n1 ; CCc1ccc2c(P(C)(C)=O)c(Nc3nc(Nc4cc(CC)c(N5CCC(N6CCN(C7CN(c8cccc9c8n(C)c(=O)n9C8CCC(=O)NC8=O)C7)CC6)CC5)cc4OC)ncc3Br)ccc2n1]

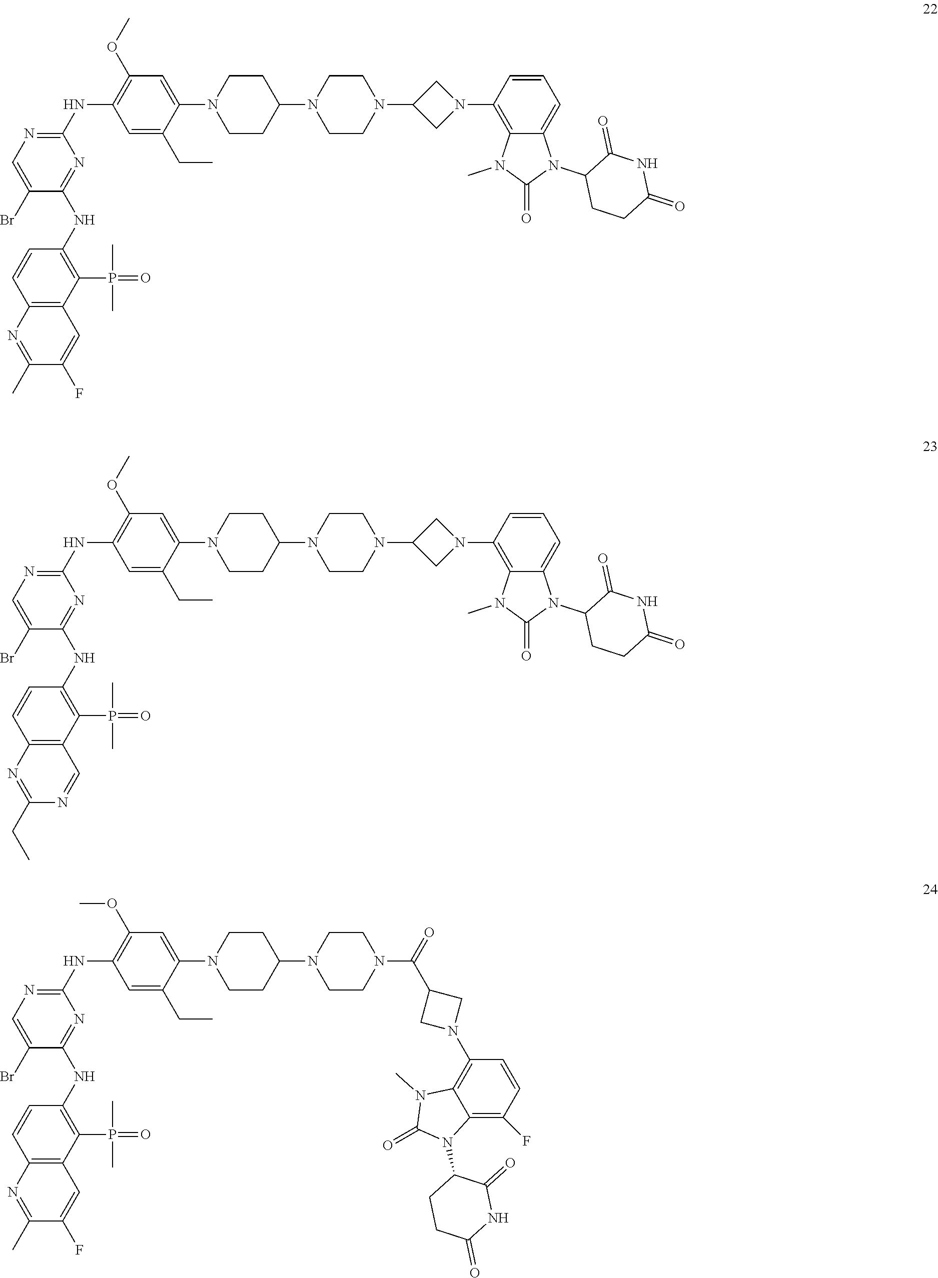 [SMILES: CCc1cc(Nc2ncc(Br)c(Nc3ccc4nc(C)c(F)cc4c3P(C)(C)=O)n2)c(OC)cc1N1CCC(N2CCN(C3CN(c4cccc5c4n(C)c(=O)n5C4CCC(=O)NC4=O)C3)CC2)CC1 ; CCc1ncc2c(P(C)(C)=O)c(Nc3nc(Nc4cc(CC)c(N5CCC(N6CCN(C7CN(c8cccc9c8n(C)c(=O)n9C8CCC(=O)NC8=O)C7)CC6)CC5)cc4OC)ncc3Br)ccc2n1 ; CCc1cc(Nc2ncc(Br)c(Nc3ccc4nc(C)c(F)cc4c3P(C)(C)=O)n2)c(OC)cc1N1CCC(N2CCN(C(=O)C3CN(c4ccc(F)c5c4n(C)c(=O)n5[C@H]4CCC(=O)NC4=O)C3)CC2)CC1]

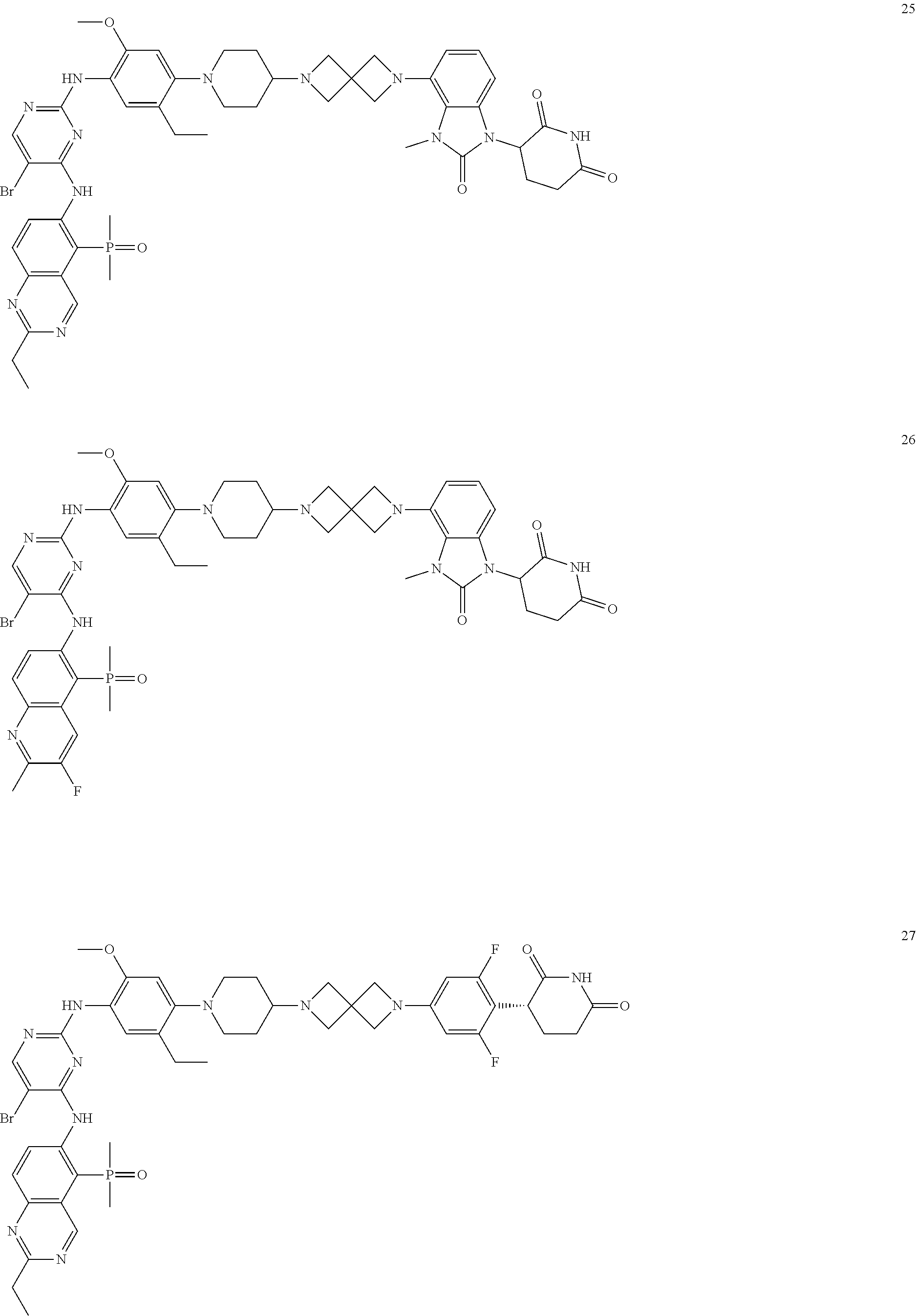 [SMILES: CCc1ncc2c(P(C)(C)=O)c(Nc3nc(Nc4cc(CC)c(N5CCC(N6CC7(CN(c8cccc9c8n(C)c(=O)n9C8CCC(=O)NC8=O)C7)C6)CC5)cc4OC)ncc3Br)ccc2n1 ; CCc1cc(Nc2ncc(Br)c(Nc3ccc4nc(C)c(F)cc4c3P(C)(C)=O)n2)c(OC)cc1N1CCC(N2CC3(CN(c4cccc5c4n(C)c(=O)n5C4CCC(=O)NC4=O)C3)C2)CC1 ; CCc1ncc2c(P(C)(C)=O)c(Nc3nc(Nc4cc(CC)c(N5CCC(N6CC7(CN(c8cc(F)c([C@H]9CCC(=O)NC9=O)c(F)c8)C7)C6)CC5)cc4OC)ncc3Br)ccc2n1]

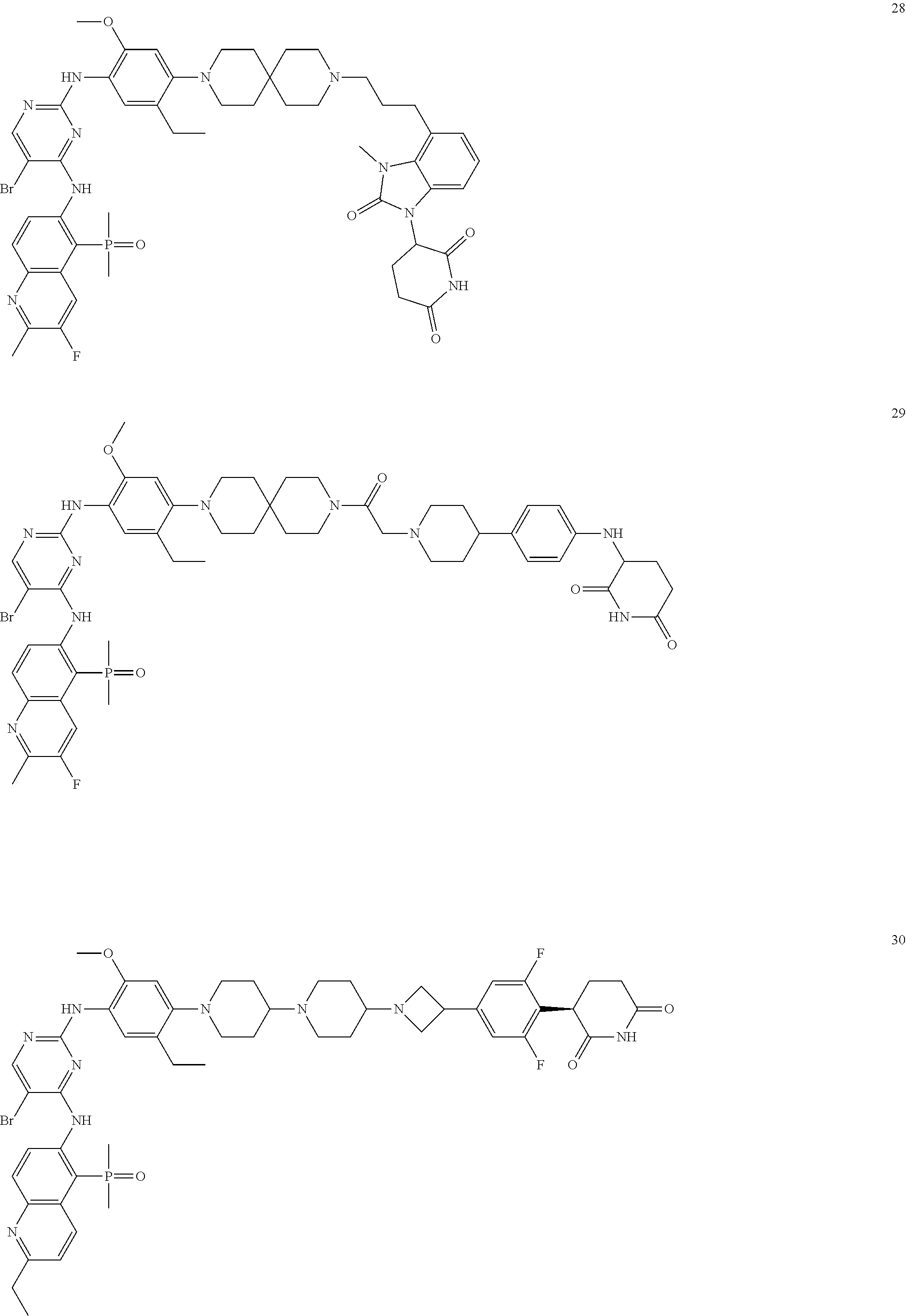 [SMILES: CCc1cc(Nc2ncc(Br)c(Nc3ccc4nc(C)c(F)cc4c3P(C)(C)=O)n2)c(OC)cc1N1CCC2(CCN(CCCc3cccc4c3n(C)c(=O)n4C3CCC(=O)NC3=O)CC2)CC1 ; CCc1cc(Nc2ncc(Br)c(Nc3ccc4nc(C)c(F)cc4c3P(C)(C)=O)n2)c(OC)cc1N1CCC2(CCN(C(=O)CN3CCC(c4ccc(NC5CCC(=O)NC5=O)cc4)CC3)CC2)CC1 ; CCc1ccc2c(P(C)(C)=O)c(Nc3nc(Nc4cc(CC)c(N5CCC(N6CCC(N7CC(c8cc(F)c([C@H]9CCC(=O)NC9=O)c(F)c8)C7)CC6)CC5)cc4OC)ncc3Br)ccc2n1]

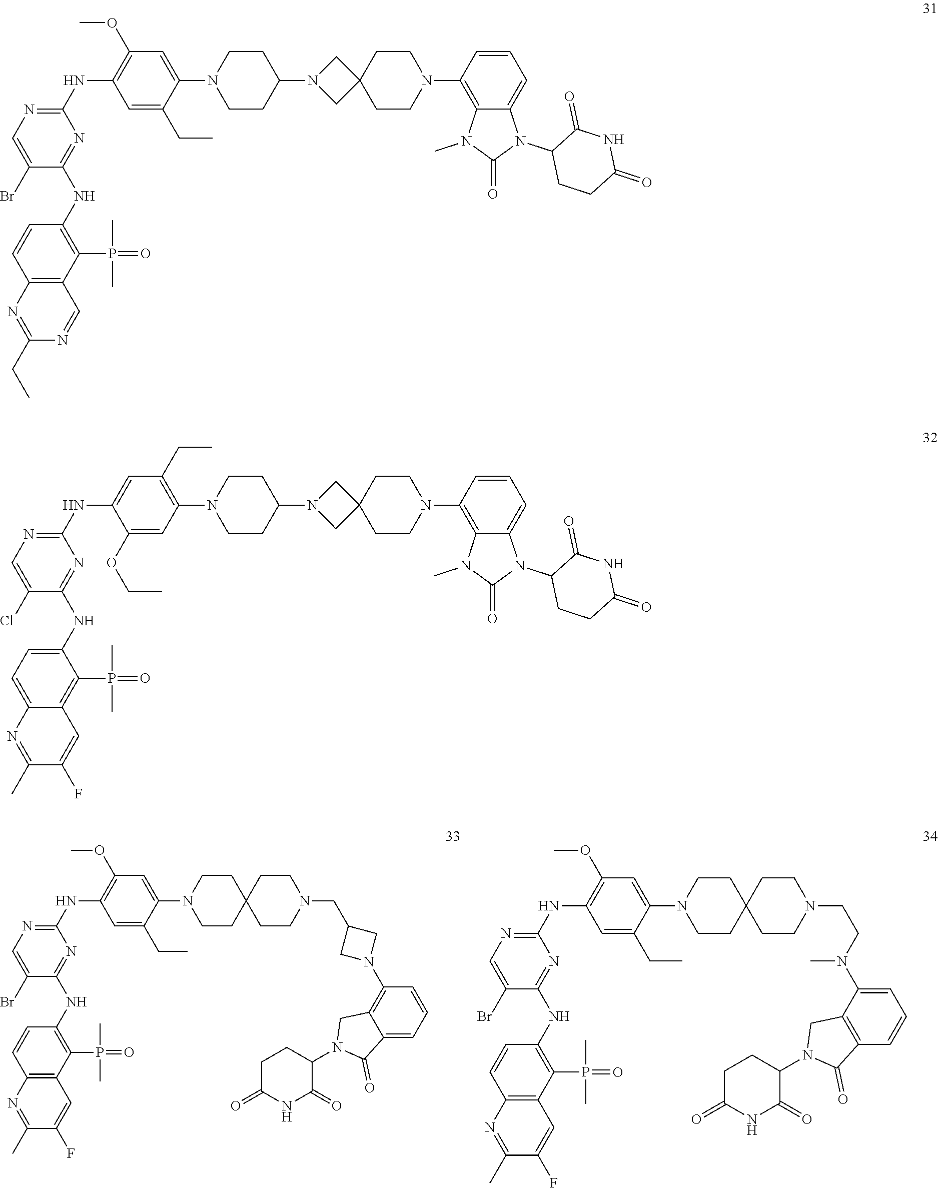 [SMILES: CCc1ncc2c(P(C)(C)=O)c(Nc3nc(Nc4cc(CC)c(N5CCC(N6CC7(CCN(c8cccc9c8n(C)c(=O)n9C8CCC(=O)NC8=O)CC7)C6)CC5)cc4OC)ncc3Br)ccc2n1 ; CCOc1cc(N2CCC(N3CC4(CCN(c5cccc6c5n(C)c(=O)n6C5CCC(=O)NC5=O)CC4)C3)CC2)c(CC)cc1Nc1ncc(Cl)c(Nc2ccc3nc(C)c(F)cc3c2P(C)(C)=O)n1 ; CCc1cc(Nc2ncc(Br)c(Nc3ccc4nc(C)c(F)cc4c3P(C)(C)=O)n2)c(OC)cc1N1CCC2(CCN(CC3CN(c4cccc5c4CN(C4CCC(=O)NC4=O)C5=O)C3)CC2)CC1 ; CCc1cc(Nc2ncc(Br)c(Nc3ccc4nc(C)c(F)cc4c3P(C)(C)=O)n2)c(OC)cc1N1CCC2(CCN(CCN(C)c3cccc4c3CN(C3CCC(=O)NC3=O)C4=O)CC2)CC1]

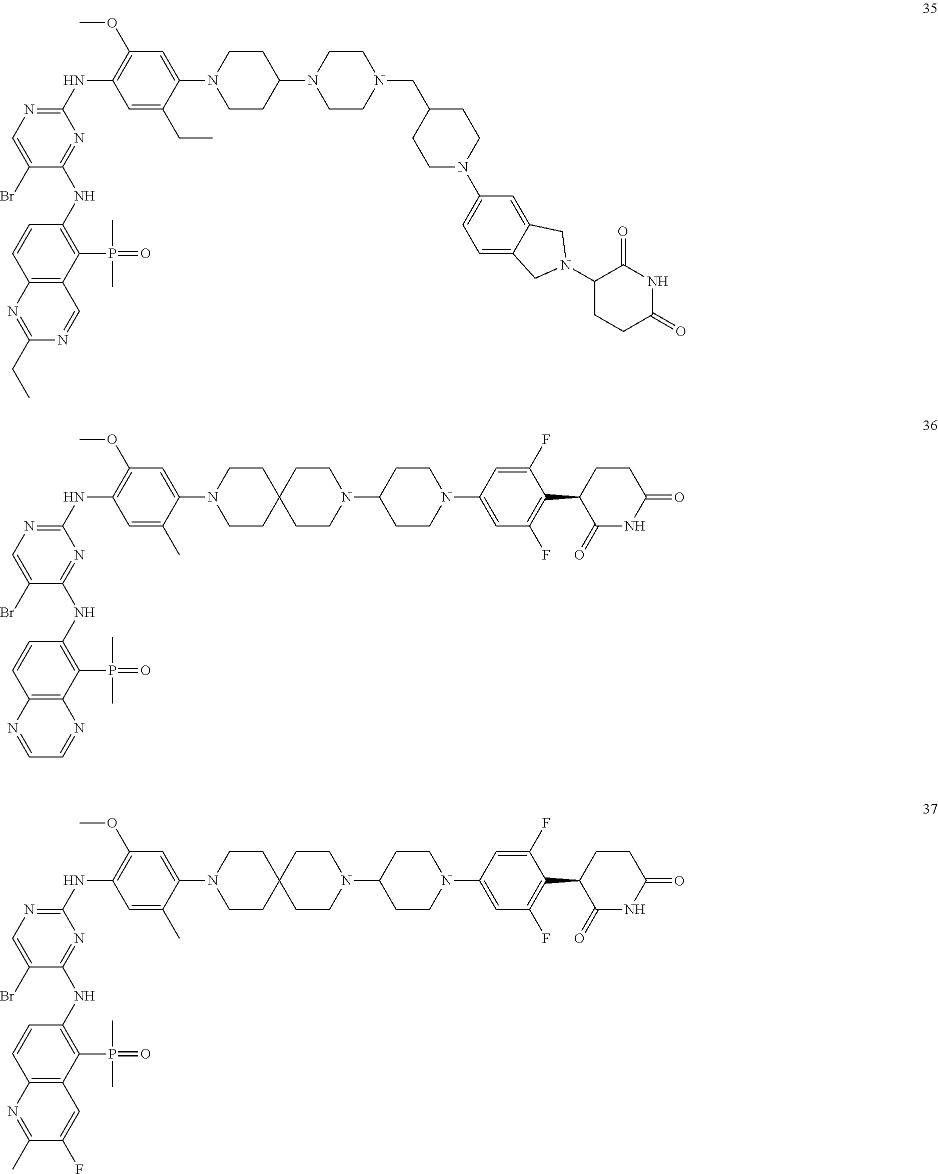 [SMILES: CCc1ncc2c(P(C)(C)=O)c(Nc3nc(Nc4cc(CC)c(N5CCC(N6CCN(CC7CCN(c8ccc9c(c8)CN(C8CCC(=O)NC8=O)C9)CC7)CC6)CC5)cc4OC)ncc3Br)ccc2n1 ; COc1cc(N2CCC3(CC2)CCN(C2CCN(c4cc(F)c([C@H]5CCC(=O)NC5=O)c(F)c4)CC2)CC3)c(C)cc1Nc1ncc(Br)c(Nc2ccc3nccnc3c2P(C)(C)=O)n1 ; COc1cc(N2CCC3(CC2)CCN(C2CCN(c4cc(F)c([C@H]5CCC(=O)NC5=O)c(F)c4)CC2)CC3)c(C)cc1Nc1ncc(Br)c(Nc2ccc3nc(C)c(F)cc3c2P(C)(C)=O)n1]

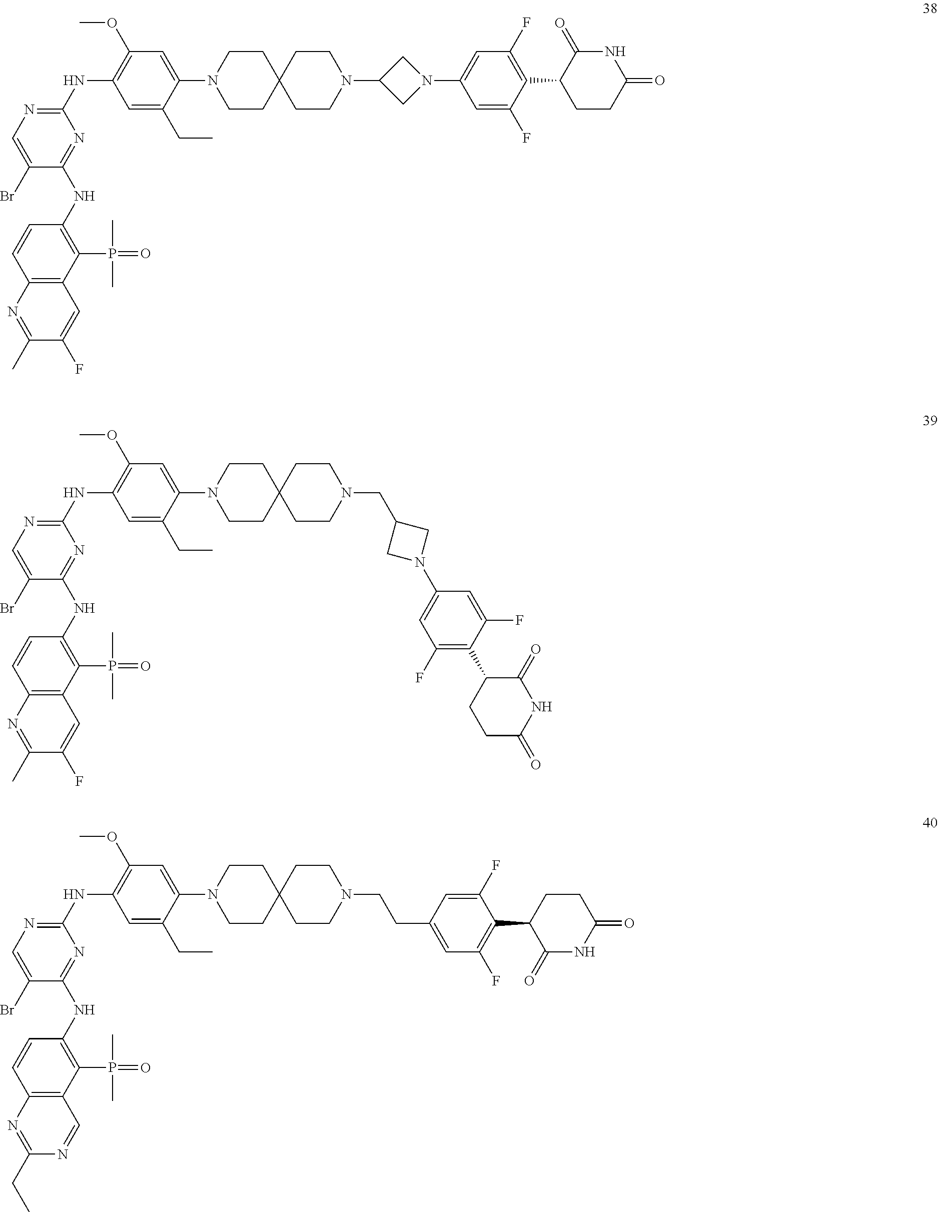 [SMILES: CCc1cc(Nc2ncc(Br)c(Nc3ccc4nc(C)c(F)cc4c3P(C)(C)=O)n2)c(OC)cc1N1CCC2(CC1)CCN(C1CN(c3cc(F)c([C@H]4CCC(=O)NC4=O)c(F)c3)C1)CC2 ; CCc1cc(Nc2ncc(Br)c(Nc3ccc4nc(C)c(F)cc4c3P(C)(C)=O)n2)c(OC)cc1N1CCC2(CCN(CC3CN(c4cc(F)c([C@H]5CCC(=O)NC5=O)c(F)c4)C3)CC2)CC1 ; CCc1ncc2c(P(C)(C)=O)c(Nc3nc(Nc4cc(CC)c(N5CCC6(CCN(CCc7cc(F)c([C@H]8CCC(=O)NC8=O)c(F)c7)CC6)CC5)cc4OC)ncc3Br)ccc2n1]

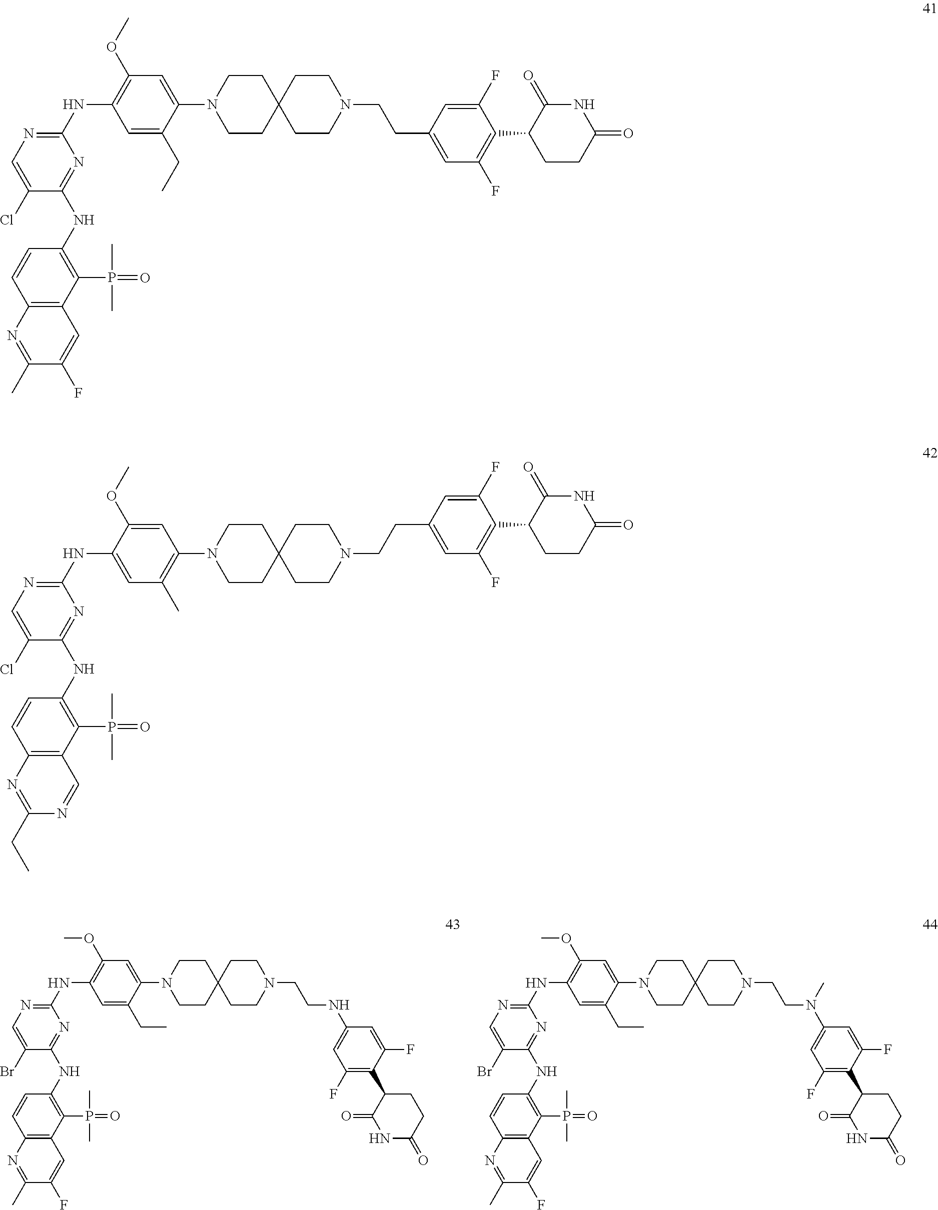 [SMILES: CCc1cc(Nc2ncc(Cl)c(Nc3ccc4nc(C)c(F)cc4c3P(C)(C)=O)n2)c(OC)cc1N1CCC2(CCN(CCc3cc(F)c([C@H]4CCC(=O)NC4=O)c(F)c3)CC2)CC1 ; CCc1ncc2c(P(C)(C)=O)c(Nc3nc(Nc4cc(C)c(N5CCC6(CCN(CCc7cc(F)c([C@H]8CCC(=O)NC8=O)c(F)c7)CC6)CC5)cc4OC)ncc3Cl)ccc2n1 ; CCc1cc(Nc2ncc(Br)c(Nc3ccc4nc(C)c(F)cc4c3P(C)(C)=O)n2)c(OC)cc1N1CCC2(CCN(CCNc3cc(F)c([C@H]4CCC(=O)NC4=O)c(F)c3)CC2)CC1 ; CCc1cc(Nc2ncc(Br)c(Nc3ccc4nc(C)c(F)cc4c3P(C)(C)=O)n2)c(OC)cc1N1CCC2(CCN(CCN(C)c3cc(F)c([C@H]4CCC(=O)NC4=O)c(F)c3)CC2)CC1]

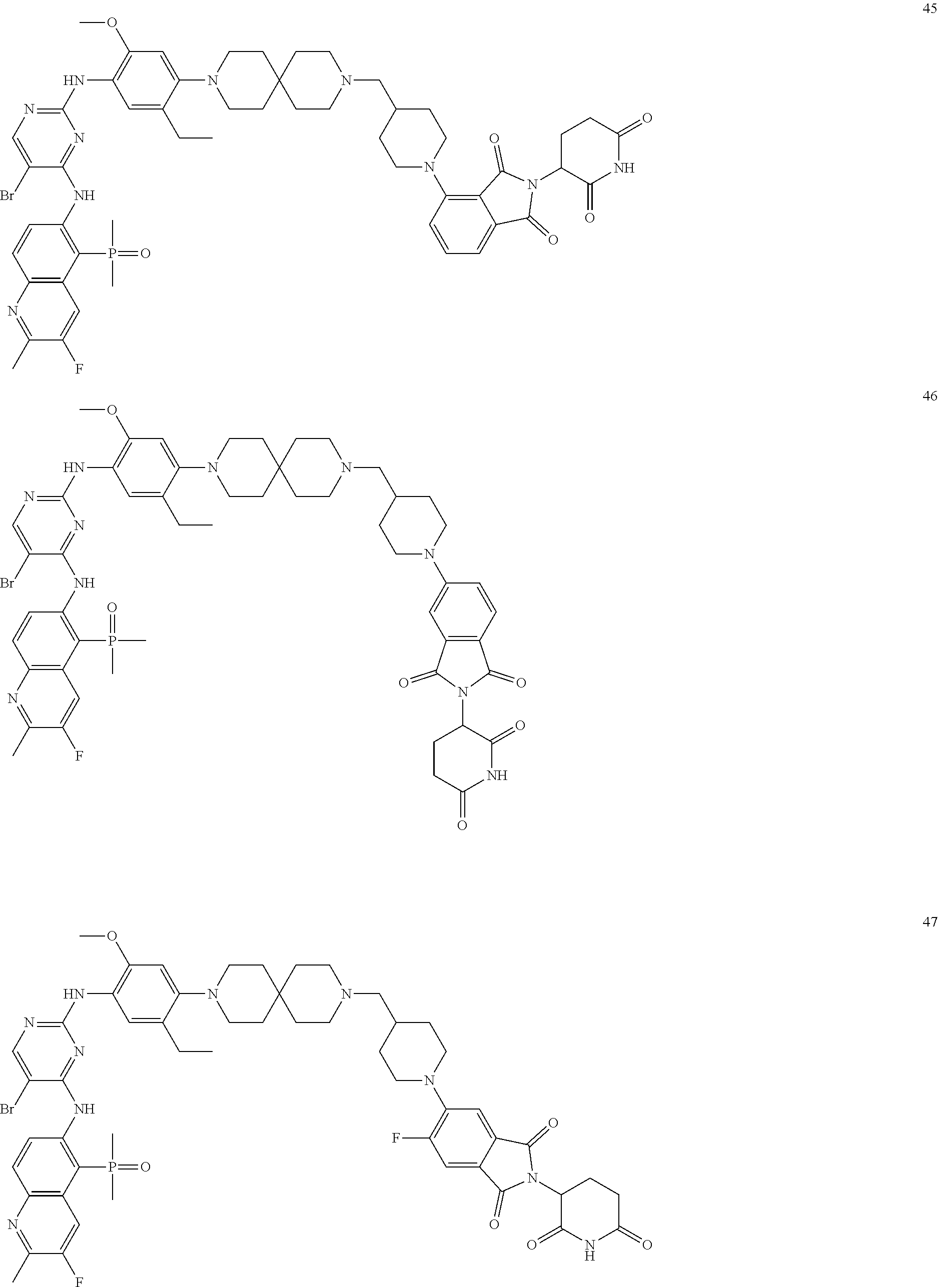

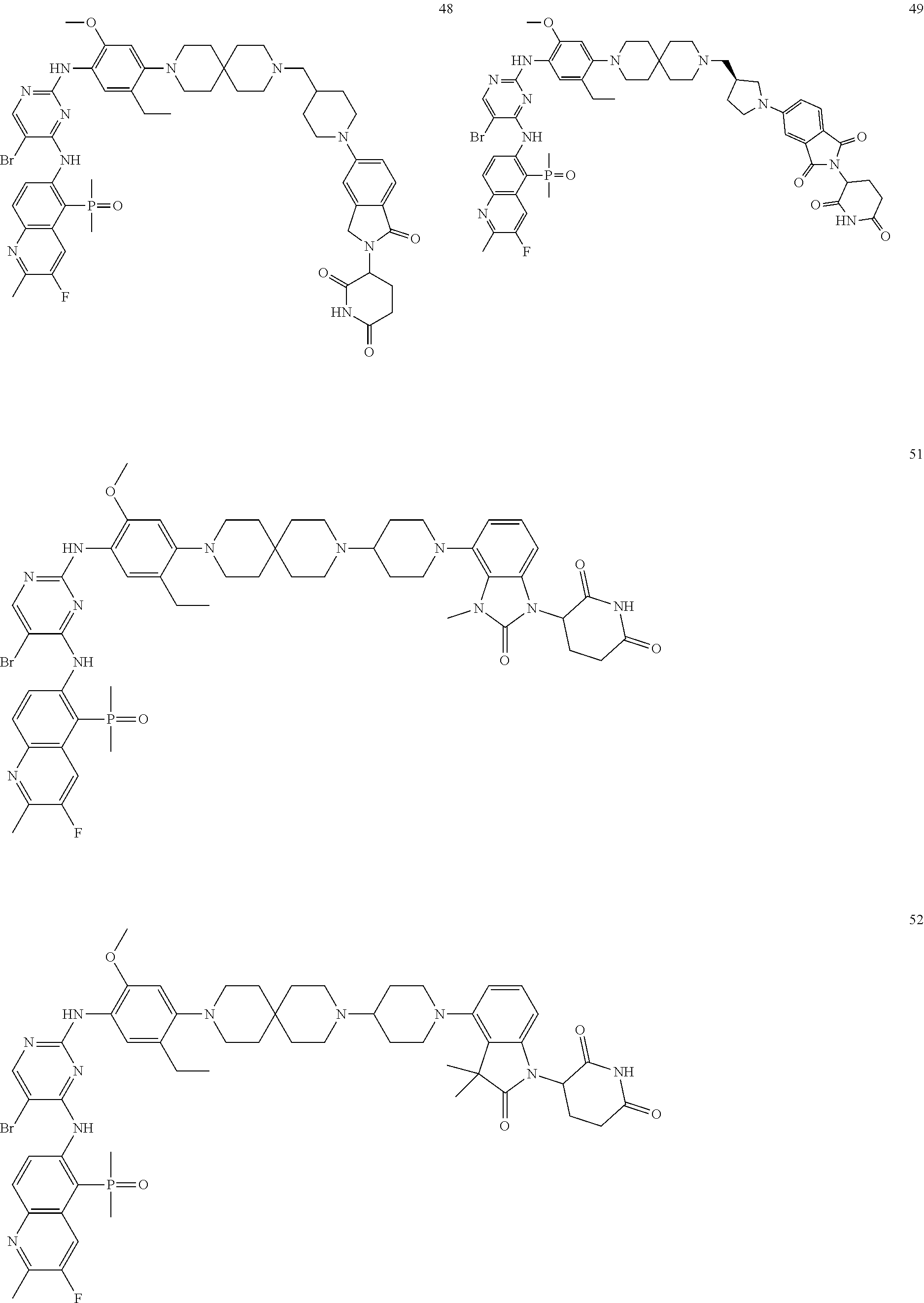

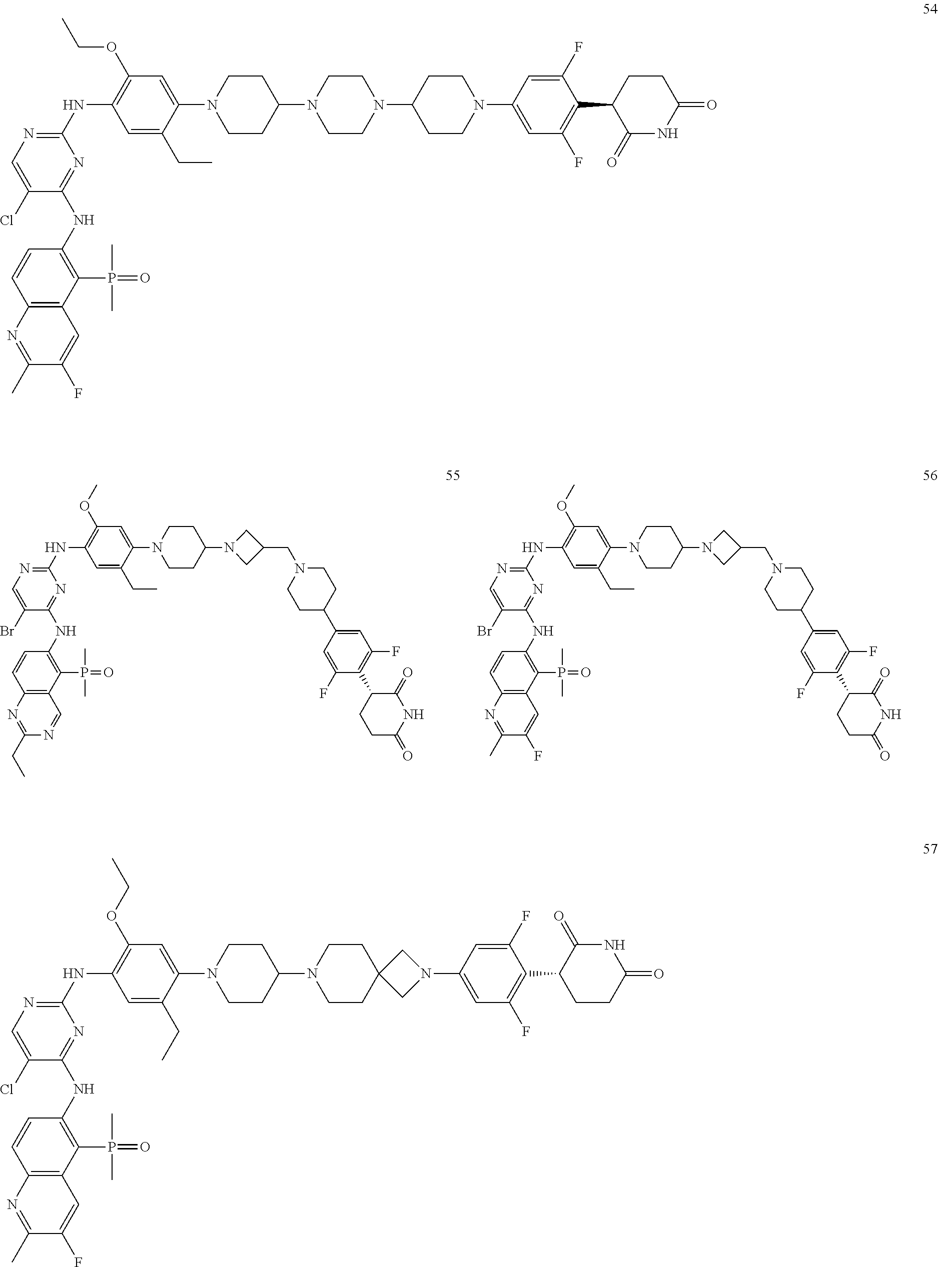

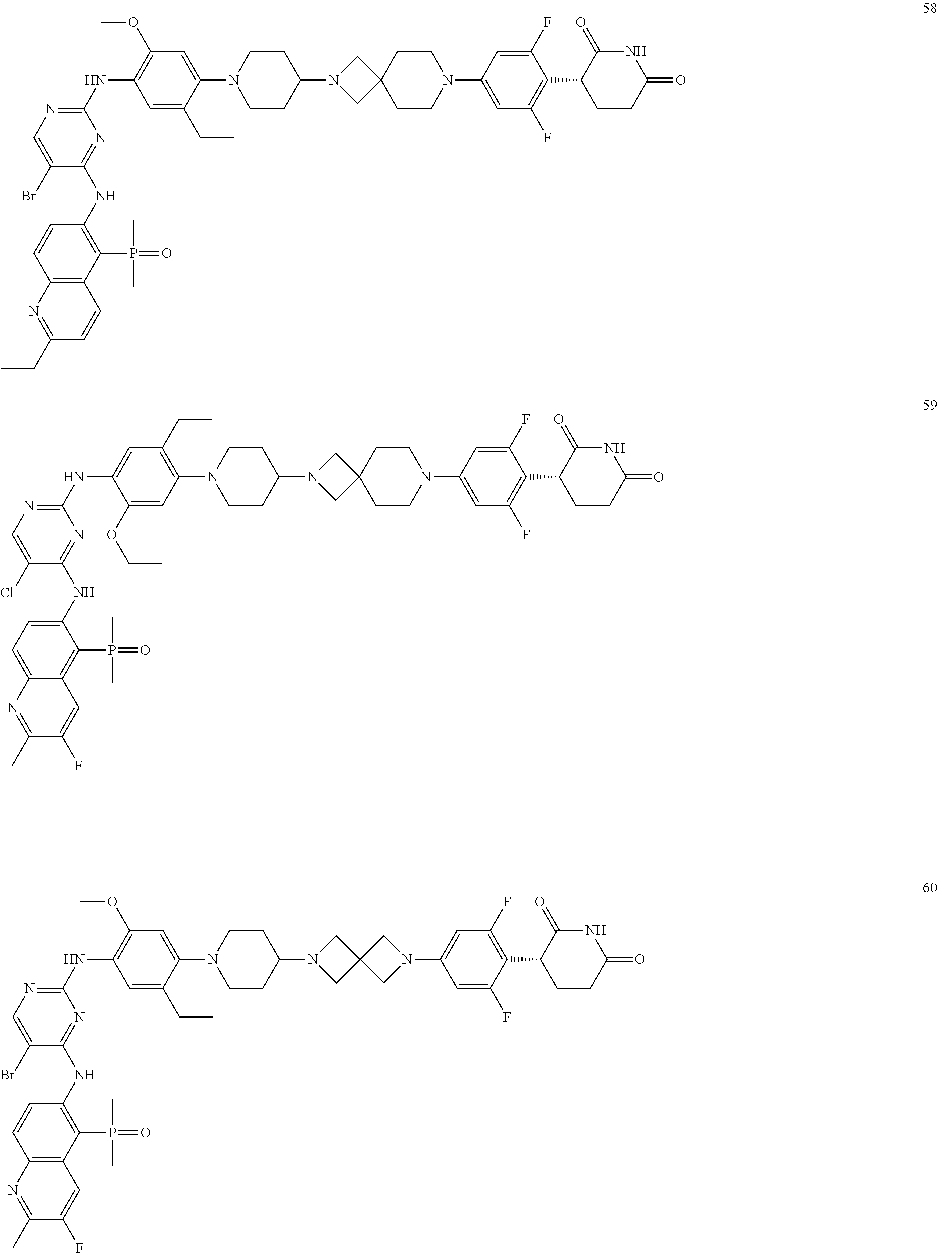 [SMILES: CCc1ccc2c(P(C)(C)=O)c(Nc3nc(Nc4cc(CC)c(N5CCC(N6CC7(CCN(c8cc(F)c([C@H]9CCC(=O)NC9=O)c(F)c8)CC7)C6)CC5)cc4OC)ncc3Br)ccc2n1 ; CCOc1cc(N2CCC(N3CC4(CCN(c5cc(F)c([C@H]6CCC(=O)NC6=O)c(F)c5)CC4)C3)CC2)c(CC)cc1Nc1ncc(Cl)c(Nc2ccc3nc(C)c(F)cc3c2P(C)(C)=O)n1 ; CCc1cc(Nc2ncc(Br)c(Nc3ccc4nc(C)c(F)cc4c3P(C)(C)=O)n2)c(OC)cc1N1CCC(N2CC3(CN(c4cc(F)c([C@H]5CCC(=O)NC5=O)c(F)c4)C3)C2)CC1]

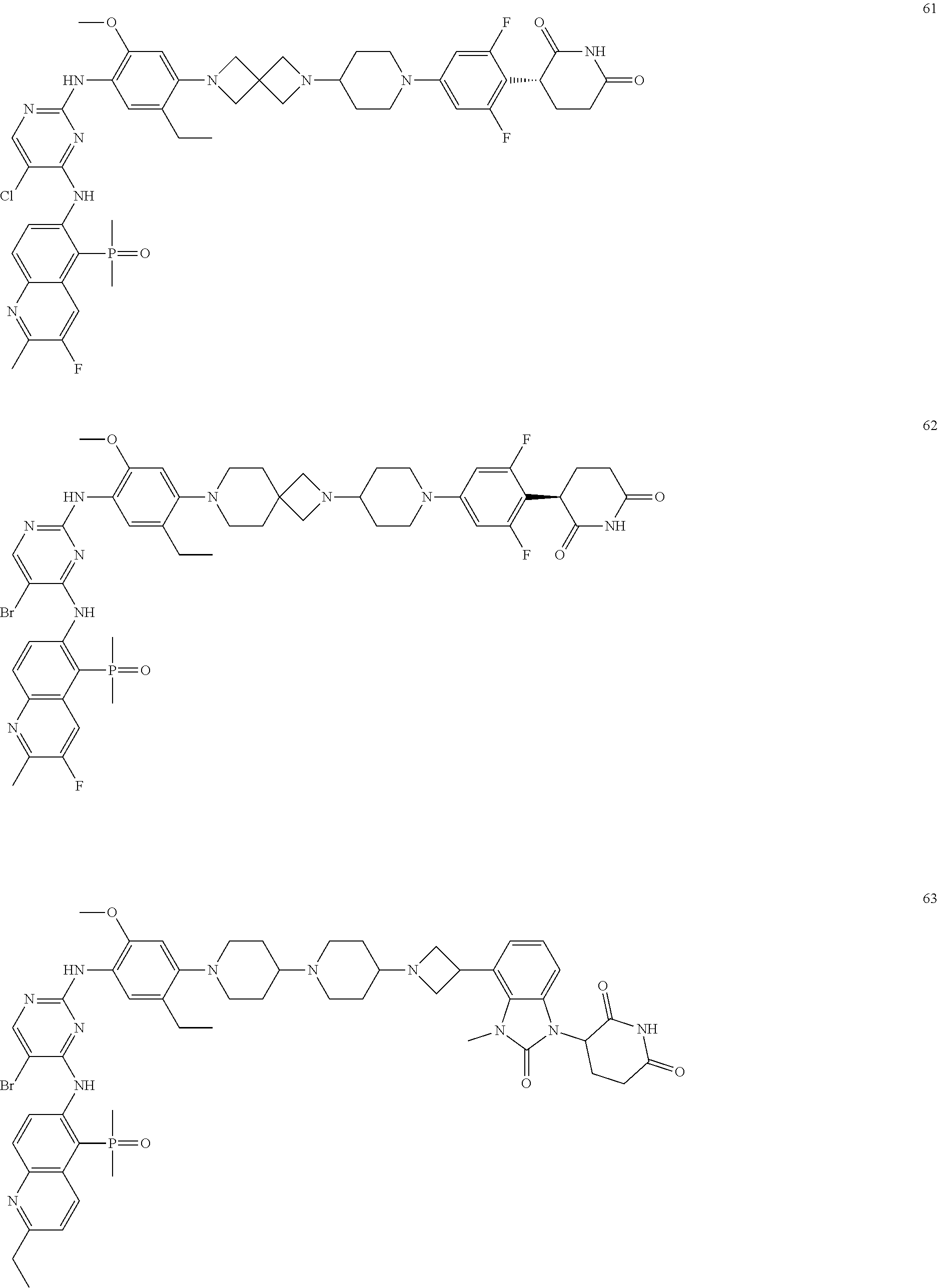 [SMILES: CCc1cc(Nc2ncc(Cl)c(Nc3ccc4nc(C)c(F)cc4c3P(C)(C)=O)n2)c(OC)cc1N1CC2(C1)CN(C1CCN(c3cc(F)c([C@H]4CCC(=O)NC4=O)c(F)c3)CC1)C2 ; CCc1cc(Nc2ncc(Br)c(Nc3ccc4nc(C)c(F)cc4c3P(C)(C)=O)n2)c(OC)cc1N1CCC2(CC1)CN(C1CCN(c3cc(F)c([C@H]4CCC(=O)NC4=O)c(F)c3)CC1)C2 ; CCc1ccc2c(P(C)(C)=O)c(Nc3nc(Nc4cc(CC)c(N5CCC(N6CCC(N7CC(c8cccc9c8n(C)c(=O)n9C8CCC(=O)NC8=O)C7)CC6)CC5)cc4OC)ncc3Br)ccc2n1]

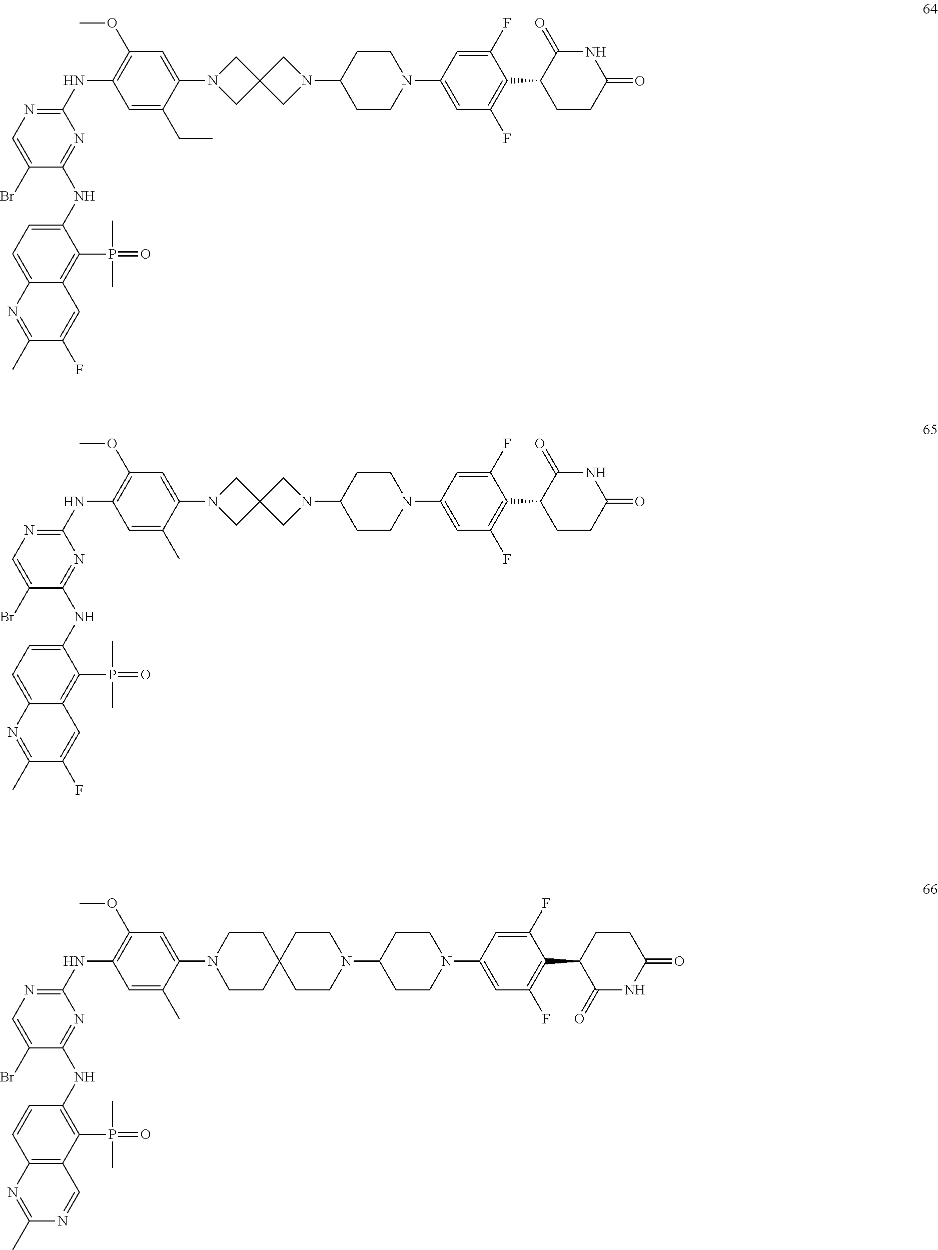 [SMILES: CCc1cc(Nc2ncc(Br)c(Nc3ccc4nc(C)c(F)cc4c3P(C)(C)=O)n2)c(OC)cc1N1CC2(C1)CN(C1CCN(c3cc(F)c([C@H]4CCC(=O)NC4=O)c(F)c3)CC1)C2 ; COc1cc(N2CC3(C2)CN(C2CCN(c4cc(F)c([C@H]5CCC(=O)NC5=O)c(F)c4)CC2)C3)c(C)cc1Nc1ncc(Br)c(Nc2ccc3nc(C)c(F)cc3c2P(C)(C)=O)n1 ; COc1cc(N2CCC3(CC2)CCN(C2CCN(c4cc(F)c([C@H]5CCC(=O)NC5=O)c(F)c4)CC2)CC3)c(C)cc1Nc1ncc(Br)c(Nc2ccc3nc(C)ncc3c2P(C)(C)=O)n1]

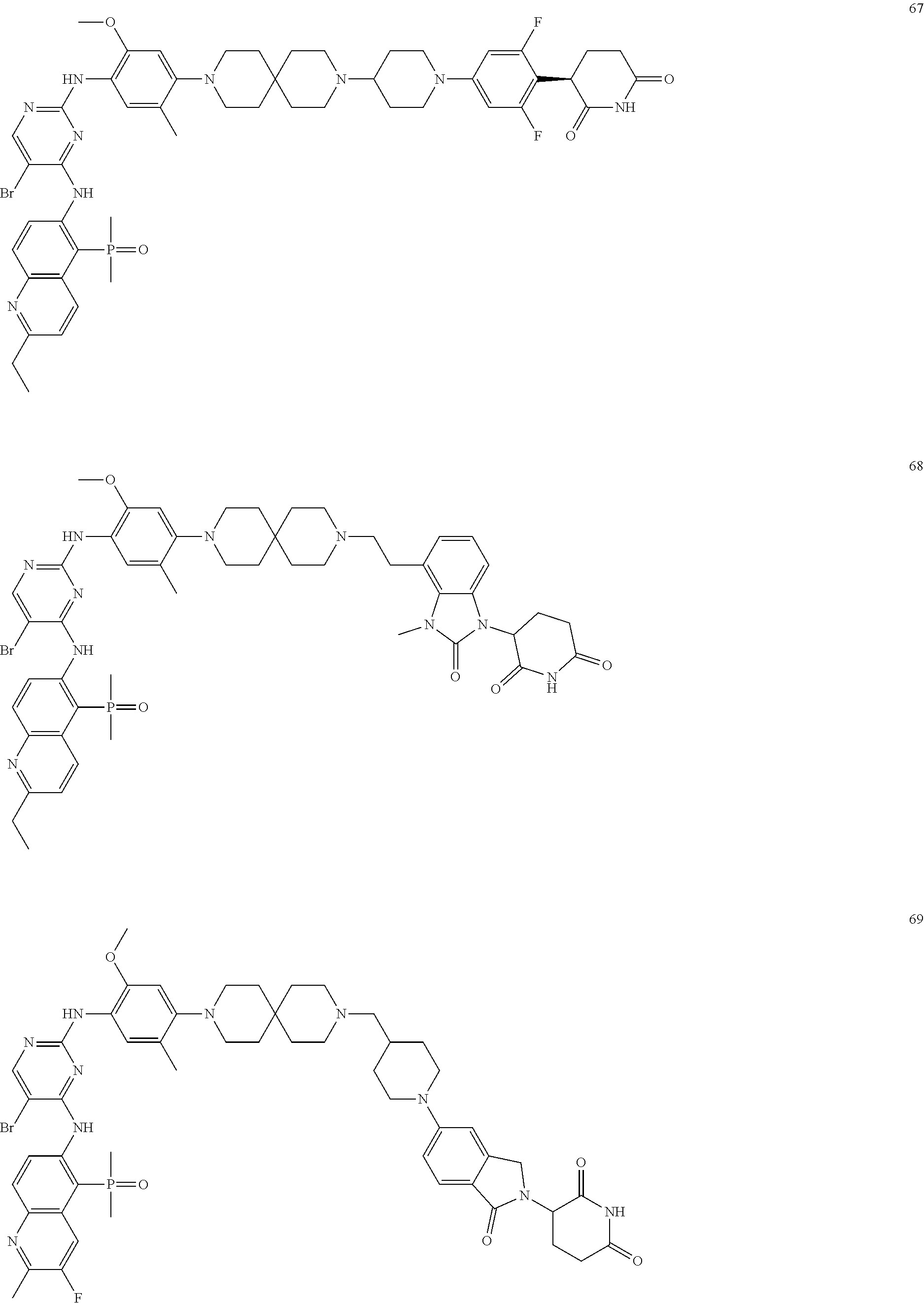 [SMILES: CCc1ccc2c(P(C)(C)=O)c(Nc3nc(Nc4cc(C)c(N5CCC6(CC5)CCN(C5CCN(c7cc(F)c([C@H]8CCC(=O)NC8=O)c(F)c7)CC5)CC6)cc4OC)ncc3Br)ccc2n1 ; CCc1ccc2c(P(C)(C)=O)c(Nc3nc(Nc4cc(C)c(N5CCC6(CCN(CCc7cccc8c7n(C)c(=O)n8C7CCC(=O)NC7=O)CC6)CC5)cc4OC)ncc3Br)ccc2n1 ; COc1cc(N2CCC3(CCN(CC4CCN(c5ccc6c(c5)CN(C5CCC(=O)NC5=O)C6=O)CC4)CC3)CC2)c(C)cc1Nc1ncc(Br)c(Nc2ccc3nc(C)c(F)cc3c2P(C)(C)=O)n1]

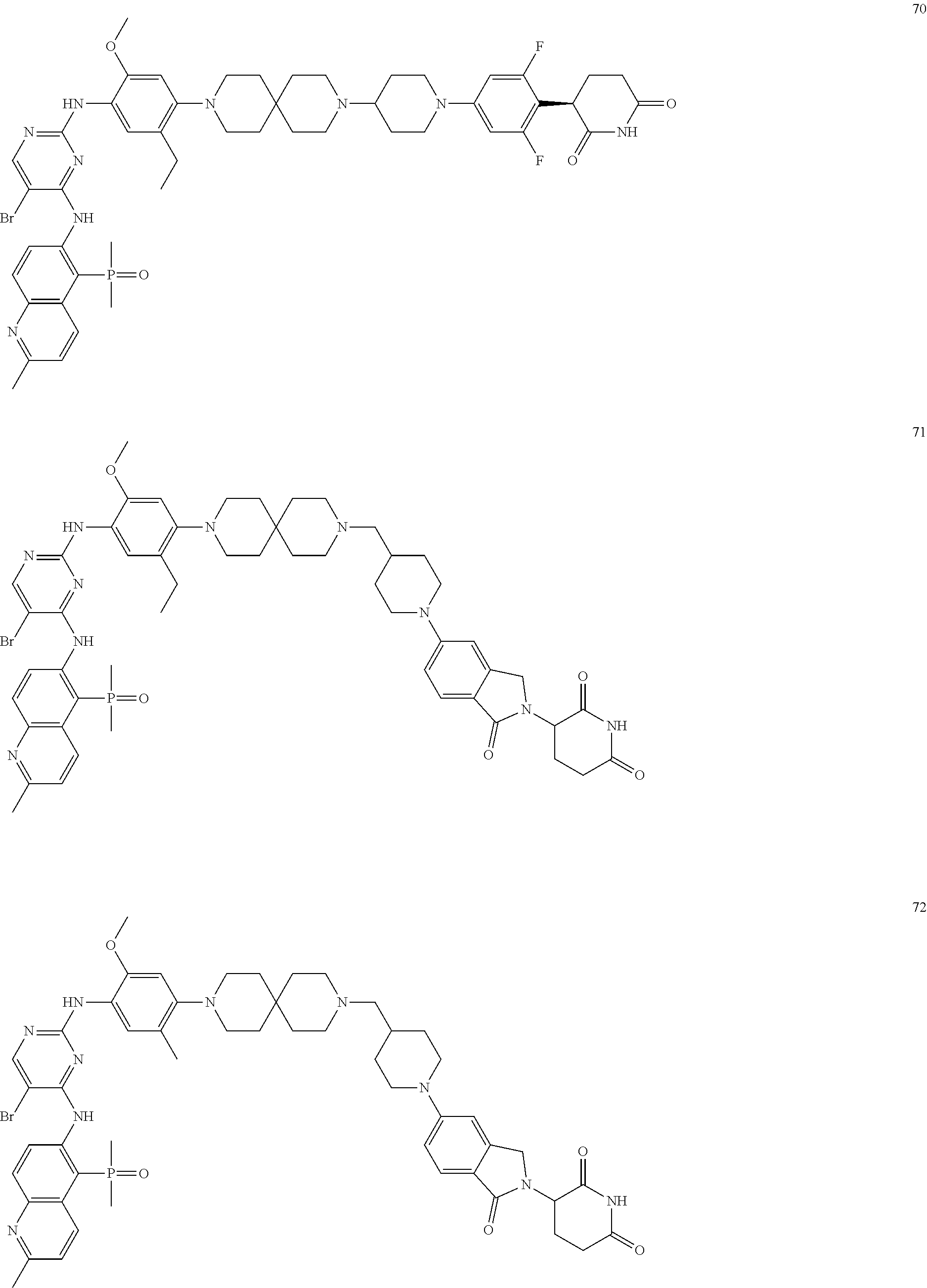 [SMILES: CCc1cc(Nc2ncc(Br)c(Nc3ccc4nc(C)ccc4c3P(C)(C)=O)n2)c(OC)cc1N1CCC2(CC1)CCN(C1CCN(c3cc(F)c([C@H]4CCC(=O)NC4=O)c(F)c3)CC1)CC2 ; CCc1cc(Nc2ncc(Br)c(Nc3ccc4nc(C)ccc4c3P(C)(C)=O)n2)c(OC)cc1N1CCC2(CCN(CC3CCN(c4ccc5c(c4)CN(C4CCC(=O)NC4=O)C5=O)CC3)CC2)CC1 ; COc1cc(N2CCC3(CCN(CC4CCN(c5ccc6c(c5)CN(C5CCC(=O)NC5=O)C6=O)CC4)CC3)CC2)c(C)cc1Nc1ncc(Br)c(Nc2ccc3nc(C)ccc3c2P(C)(C)=O)n1]

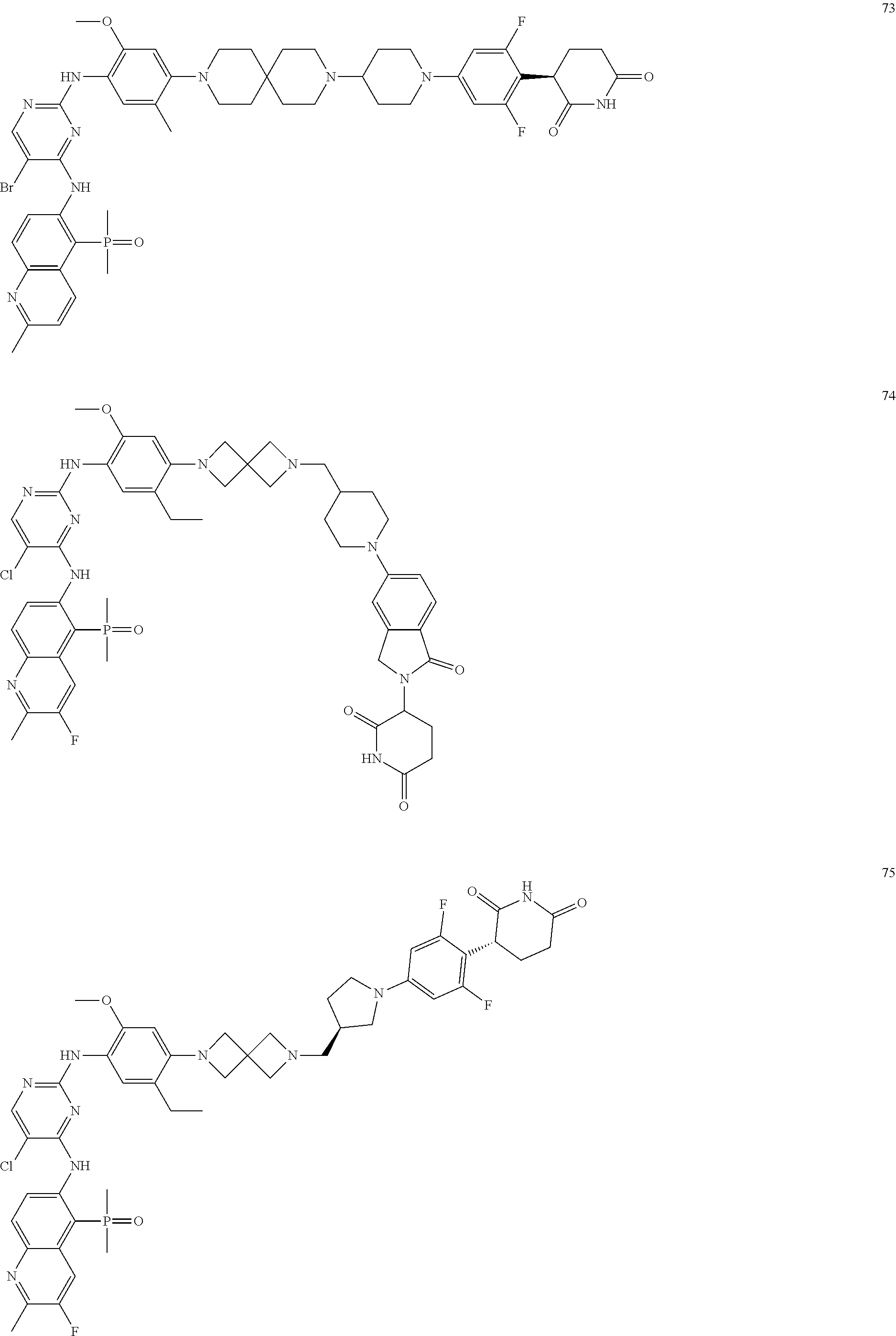 [SMILES: COc1cc(N2CCC3(CC2)CCN(C2CCN(c4cc(F)c([C@H]5CCC(=O)NC5=O)c(F)c4)CC2)CC3)c(C)cc1Nc1ncc(Br)c(Nc2ccc3nc(C)ccc3c2P(C)(C)=O)n1 ; CCc1cc(Nc2ncc(Cl)c(Nc3ccc4nc(C)c(F)cc4c3P(C)(C)=O)n2)c(OC)cc1N1CC2(CN(CC3CCN(c4ccc5c(c4)CN(C4CCC(=O)NC4=O)C5=O)CC3)C2)C1 ; CCc1cc(Nc2ncc(Cl)c(Nc3ccc4nc(C)c(F)cc4c3P(C)(C)=O)n2)c(OC)cc1N1CC2(CN(C[C@H]3CCN(c4cc(F)c([C@H]5CCC(=O)NC5=O)c(F)c4)C3)C2)C1]

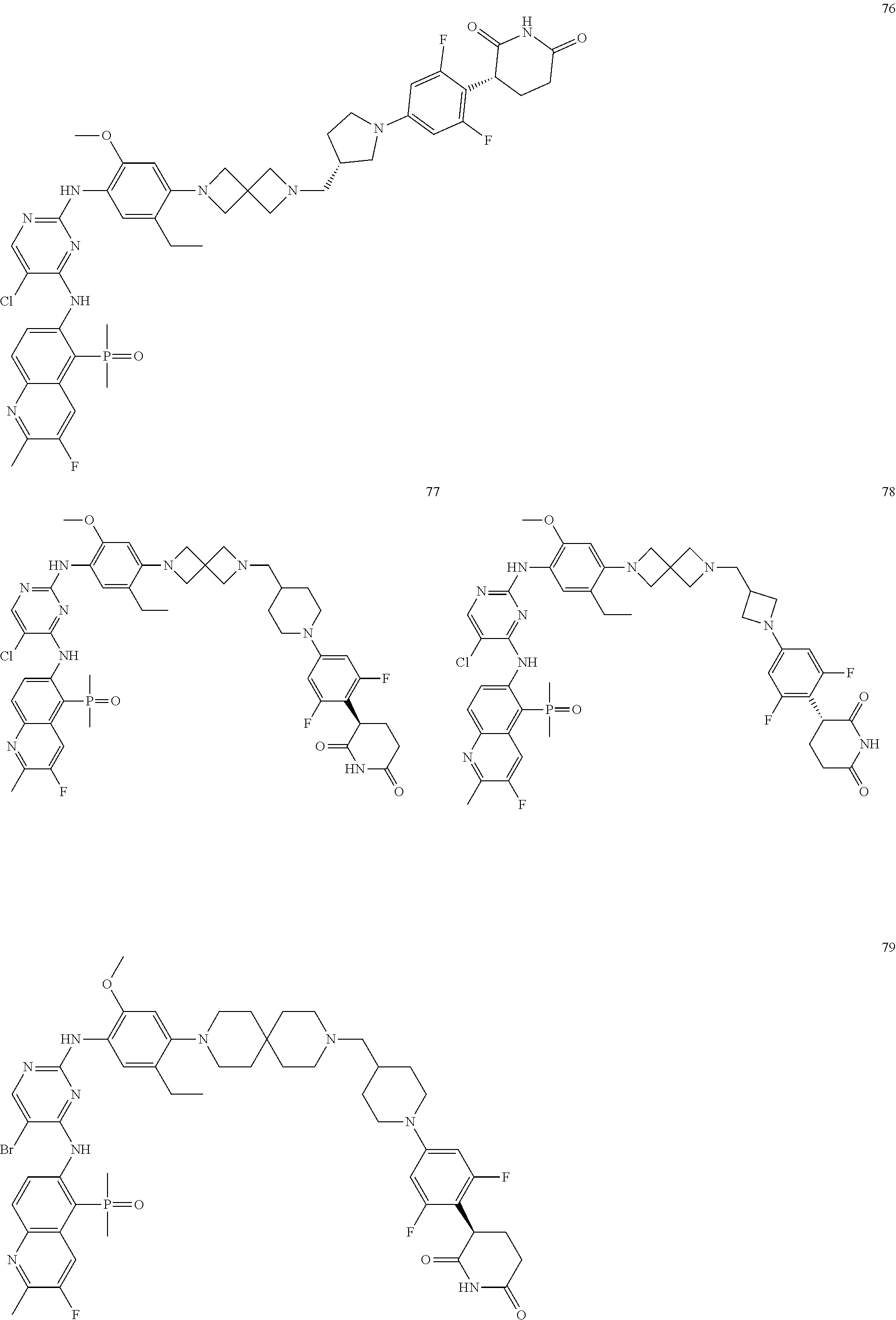 [SMILES: CCc1cc(Nc2ncc(Cl)c(Nc3ccc4nc(C)c(F)cc4c3P(C)(C)=O)n2)c(OC)cc1N1CC2(CN(C[C@@H]3CCN(c4cc(F)c([C@H]5CCC(=O)NC5=O)c(F)c4)C3)C2)C1 ; CCc1cc(Nc2ncc(Cl)c(Nc3ccc4nc(C)c(F)cc4c3P(C)(C)=O)n2)c(OC)cc1N1CC2(CN(CC3CCN(c4cc(F)c([C@H]5CCC(=O)NC5=O)c(F)c4)CC3)C2)C1 ; CCc1cc(Nc2ncc(Cl)c(Nc3ccc4nc(C)c(F)cc4c3P(C)(C)=O)n2)c(OC)cc1N1CC2(CN(CC3CN(c4cc(F)c([C@H]5CCC(=O)NC5=O)c(F)c4)C3)C2)C1 ; CCc1cc(Nc2ncc(Br)c(Nc3ccc4nc(C)c(F)cc4c3P(C)(C)=O)n2)c(OC)cc1N1CCC2(CCN(CC3CCN(c4cc(F)c([C@H]5CCC(=O)NC5=O)c(F)c4)CC3)CC2)CC1]

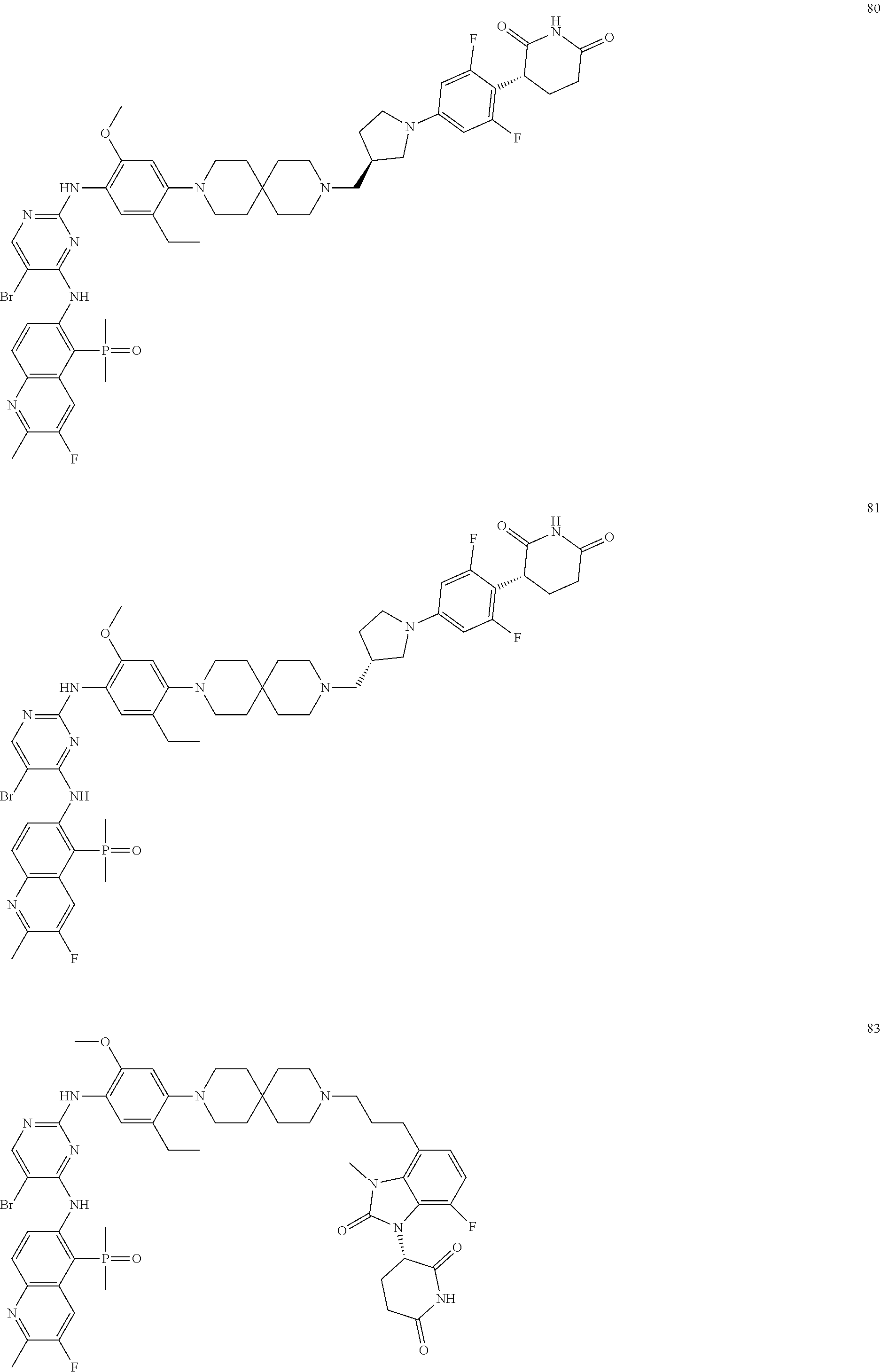

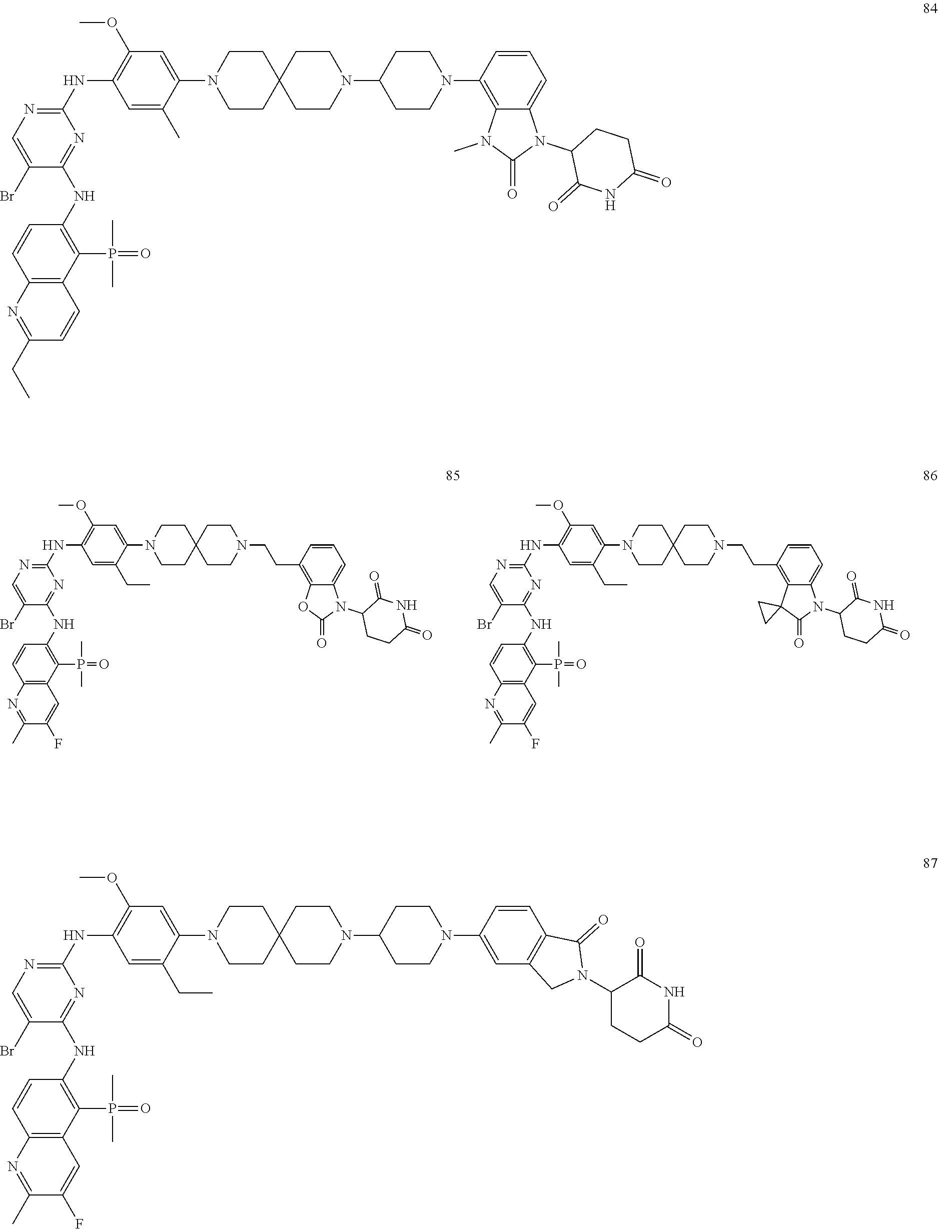 [SMILES: CCc1ccc2c(P(C)(C)=O)c(Nc3nc(Nc4cc(C)c(N5CCC6(CC5)CCN(C5CCN(c7cccc8c7n(C)c(=O)n8C7CCC(=O)NC7=O)CC5)CC6)cc4OC)ncc3Br)ccc2n1 ; CCc1cc(Nc2ncc(Br)c(Nc3ccc4nc(C)c(F)cc4c3P(C)(C)=O)n2)c(OC)cc1N1CCC2(CCN(CCc3cccc4c3oc(=O)n4C3CCC(=O)NC3=O)CC2)CC1 ; CCc1cc(Nc2ncc(Br)c(Nc3ccc4nc(C)c(F)cc4c3P(C)(C)=O)n2)c(OC)cc1N1CCC2(CCN(CCc3cccc4c3C3(CC3)C(=O)N4C3CCC(=O)NC3=O)CC2)CC1 ; CCc1cc(Nc2ncc(Br)c(Nc3ccc4nc(C)c(F)cc4c3P(C)(C)=O)n2)c(OC)cc1N1CCC2(CC1)CCN(C1CCN(c3ccc4c(c3)CN(C3CCC(=O)NC3=O)C4=O)CC1)CC2]

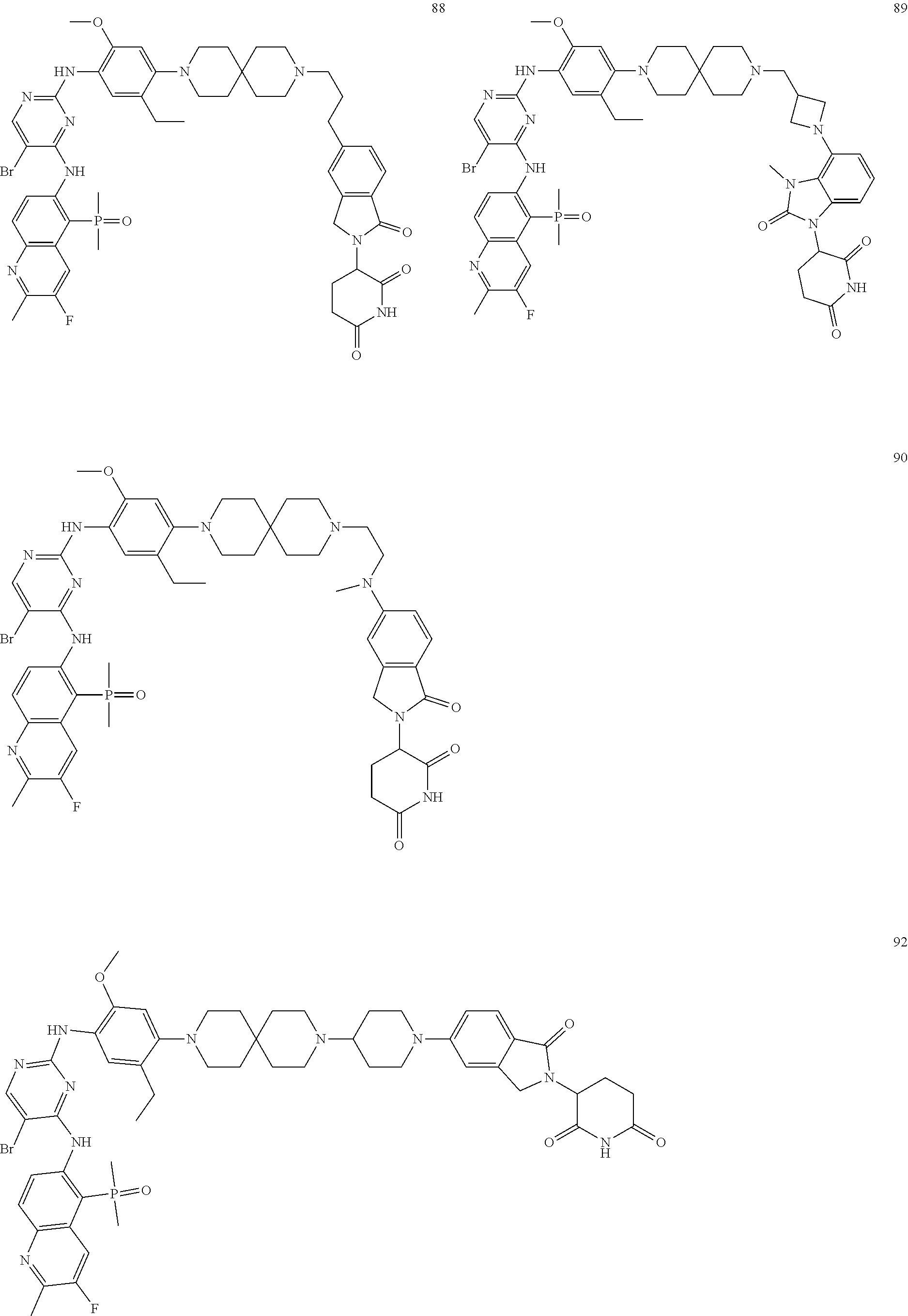 [SMILES: CCc1cc(Nc2ncc(Br)c(Nc3ccc4nc(C)c(F)cc4c3P(C)(C)=O)n2)c(OC)cc1N1CCC2(CCN(CCCc3ccc4c(c3)CN(C3CCC(=O)NC3=O)C4=O)CC2)CC1 ; CCc1cc(Nc2ncc(Br)c(Nc3ccc4nc(C)c(F)cc4c3P(C)(C)=O)n2)c(OC)cc1N1CCC2(CCN(CC3CN(c4cccc5c4n(C)c(=O)n5C4CCC(=O)NC4=O)C3)CC2)CC1 ; CCc1cc(Nc2ncc(Br)c(Nc3ccc4nc(C)c(F)cc4c3P(C)(C)=O)n2)c(OC)cc1N1CCC2(CCN(CCN(C)c3ccc4c(c3)CN(C3CCC(=O)NC3=O)C4=O)CC2)CC1 ; CCc1cc(Nc2ncc(Br)c(Nc3ccc4nc(C)c(F)cc4c3P(C)(C)=O)n2)c(OC)cc1N1CCC2(CC1)CCN(C1CCN(c3ccc4c(c3)CN(C3CCC(=O)NC3=O)C4=O)CC1)CC2]

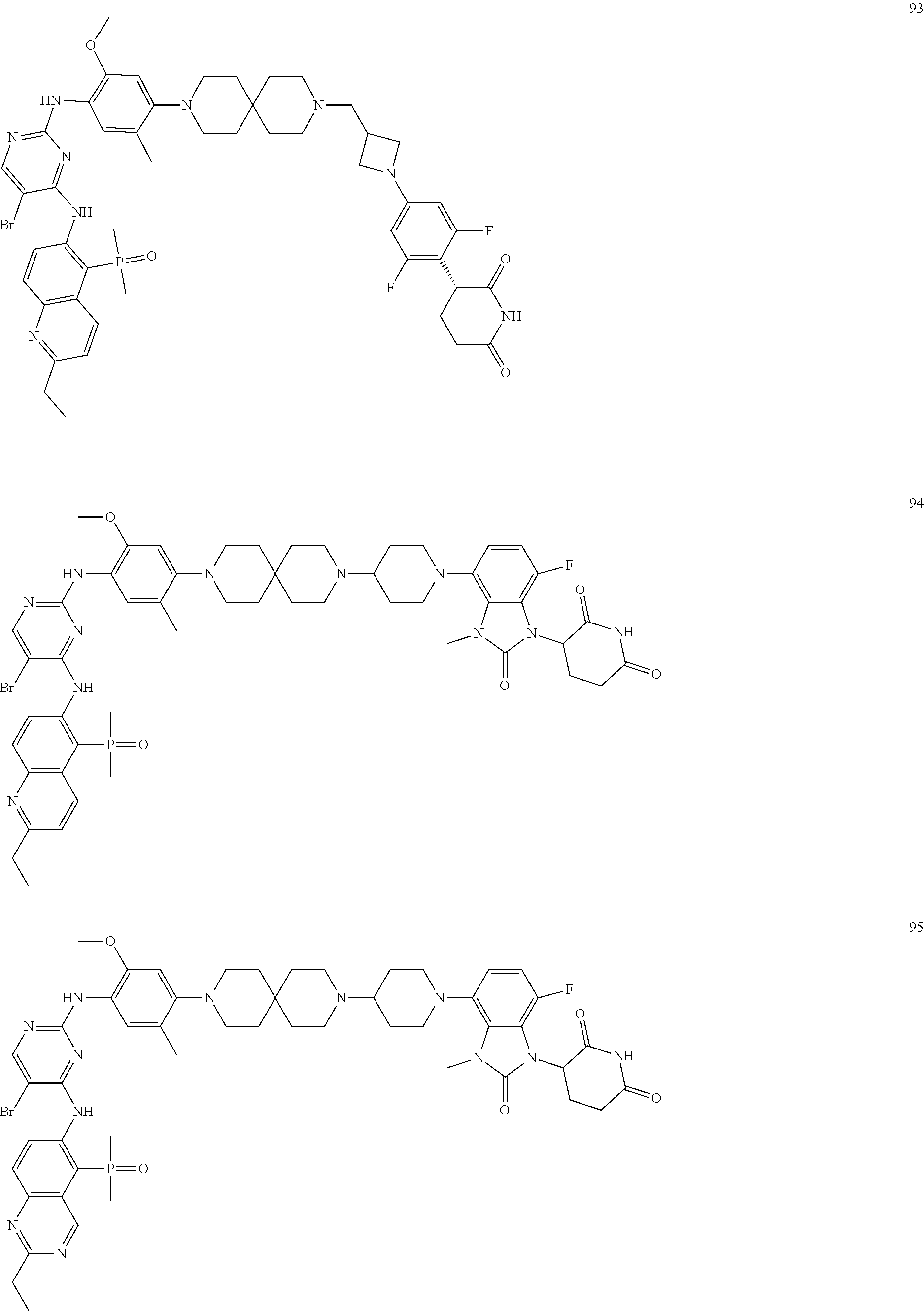 [SMILES: CCc1ccc2c(P(C)(C)=O)c(Nc3nc(Nc4cc(C)c(N5CCC6(CCN(CC7CN(c8cc(F)c([C@H]9CCC(=O)NC9=O)c(F)c8)C7)CC6)CC5)cc4OC)ncc3Br)ccc2n1 ; CCc1ccc2c(P(C)(C)=O)c(Nc3nc(Nc4cc(C)c(N5CCC6(CC5)CCN(C5CCN(c7ccc(F)c8c7n(C)c(=O)n8C7CCC(=O)NC7=O)CC5)CC6)cc4OC)ncc3Br)ccc2n1 ; CCc1ncc2c(P(C)(C)=O)c(Nc3nc(Nc4cc(C)c(N5CCC6(CC5)CCN(C5CCN(c7ccc(F)c8c7n(C)c(=O)n8C7CCC(=O)NC7=O)CC5)CC6)cc4OC)ncc3Br)ccc2n1]

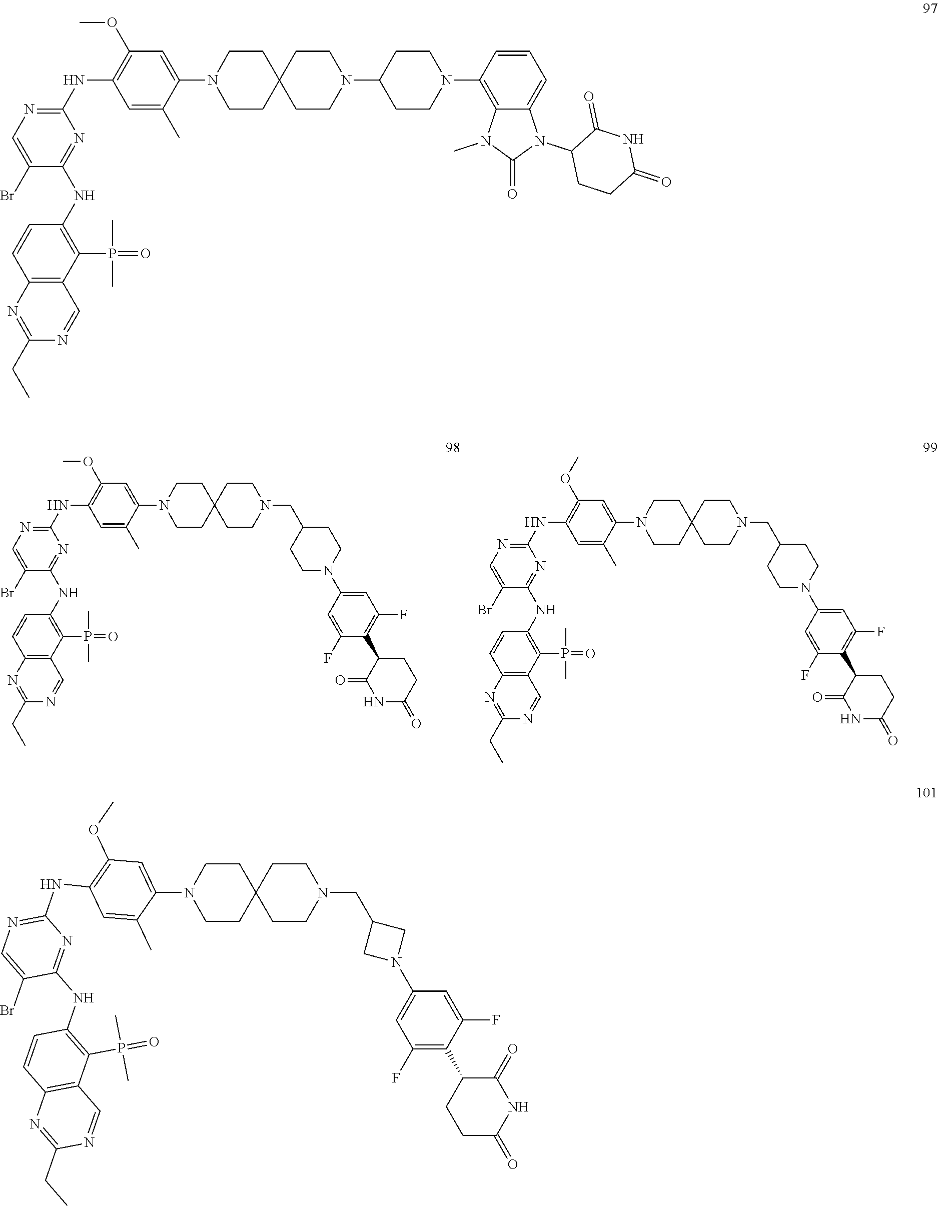 [SMILES: CCc1ncc2c(P(C)(C)=O)c(Nc3nc(Nc4cc(C)c(N5CCC6(CC5)CCN(C5CCN(c7cccc8c7n(C)c(=O)n8C7CCC(=O)NC7=O)CC5)CC6)cc4OC)ncc3Br)ccc2n1 ; CCc1ncc2c(P(C)(C)=O)c(Nc3nc(Nc4cc(C)c(N5CCC6(CCN(CC7CCN(c8cc(F)c([C@H]9CCC(=O)NC9=O)c(F)c8)CC7)CC6)CC5)cc4OC)ncc3Br)ccc2n1 ; CCc1ncc2c(P(C)(C)=O)c(Nc3nc(Nc4cc(C)c(N5CCC6(CCN(CC7CCN(c8cc(F)c([C@H]9CCC(=O)NC9=O)c(F)c8)CC7)CC6)CC5)cc4OC)ncc3Br)ccc2n1 ; CCc1ncc2c(P(C)(C)=O)c(Nc3nc(Nc4cc(C)c(N5CCC6(CCN(CC7CN(c8cc(F)c([C@H]9CCC(=O)NC9=O)c(F)c8)C7)CC6)CC5)cc4OC)ncc3Br)ccc2n1]

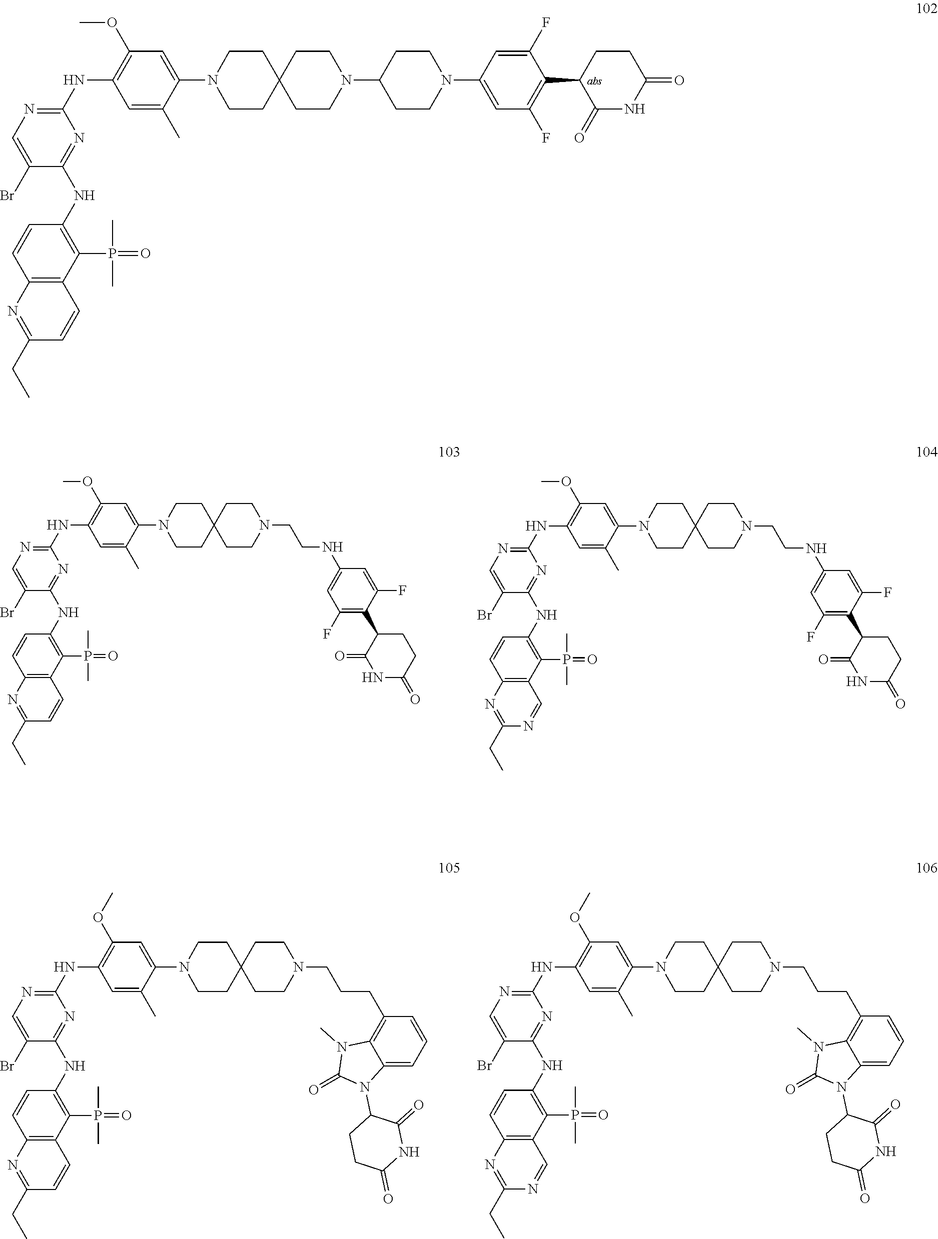

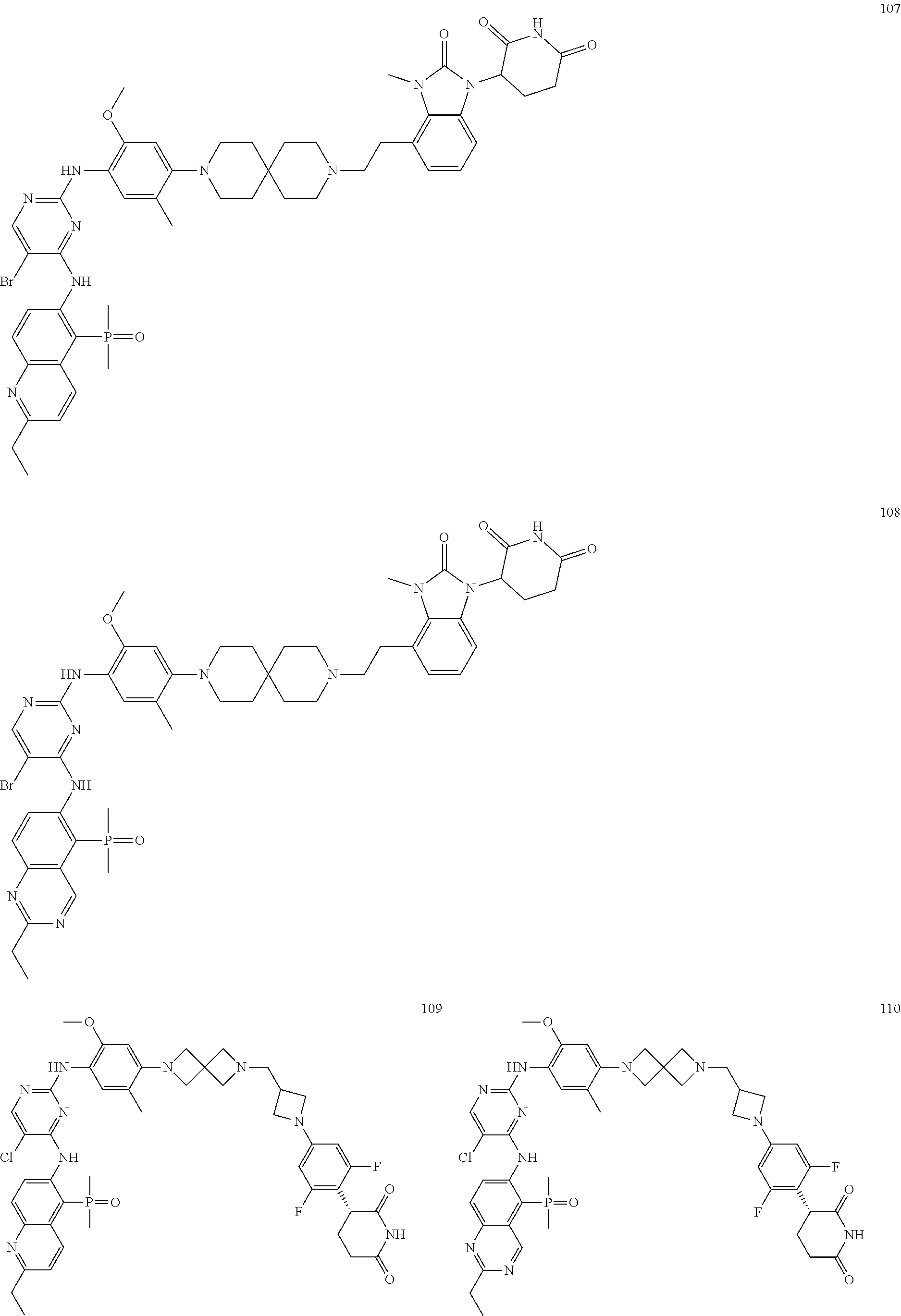 [SMILES: CCc1ccc2c(P(C)(C)=O)c(Nc3nc(Nc4cc(C)c(N5CCC6(CCN(CCc7cccc8c7n(C)c(=O)n8C7CCC(=O)NC7=O)CC6)CC5)cc4OC)ncc3Br)ccc2n1 ; CCc1ncc2c(P(C)(C)=O)c(Nc3nc(Nc4cc(C)c(N5CCC6(CCN(CCc7cccc8c7n(C)c(=O)n8C7CCC(=O)NC7=O)CC6)CC5)cc4OC)ncc3Br)ccc2n1 ; CCc1ccc2c(P(C)(C)=O)c(Nc3nc(Nc4cc(C)c(N5CC6(CN(CC7CN(c8cc(F)c([C@H]9CCC(=O)NC9=O)c(F)c8)C7)C6)C5)cc4OC)ncc3Cl)ccc2n1 ; CCc1ncc2c(P(C)(C)=O)c(Nc3nc(Nc4cc(C)c(N5CC6(CN(CC7CN(c8cc(F)c([C@H]9CCC(=O)NC9=O)c(F)c8)C7)C6)C5)cc4OC)ncc3Cl)ccc2n1]

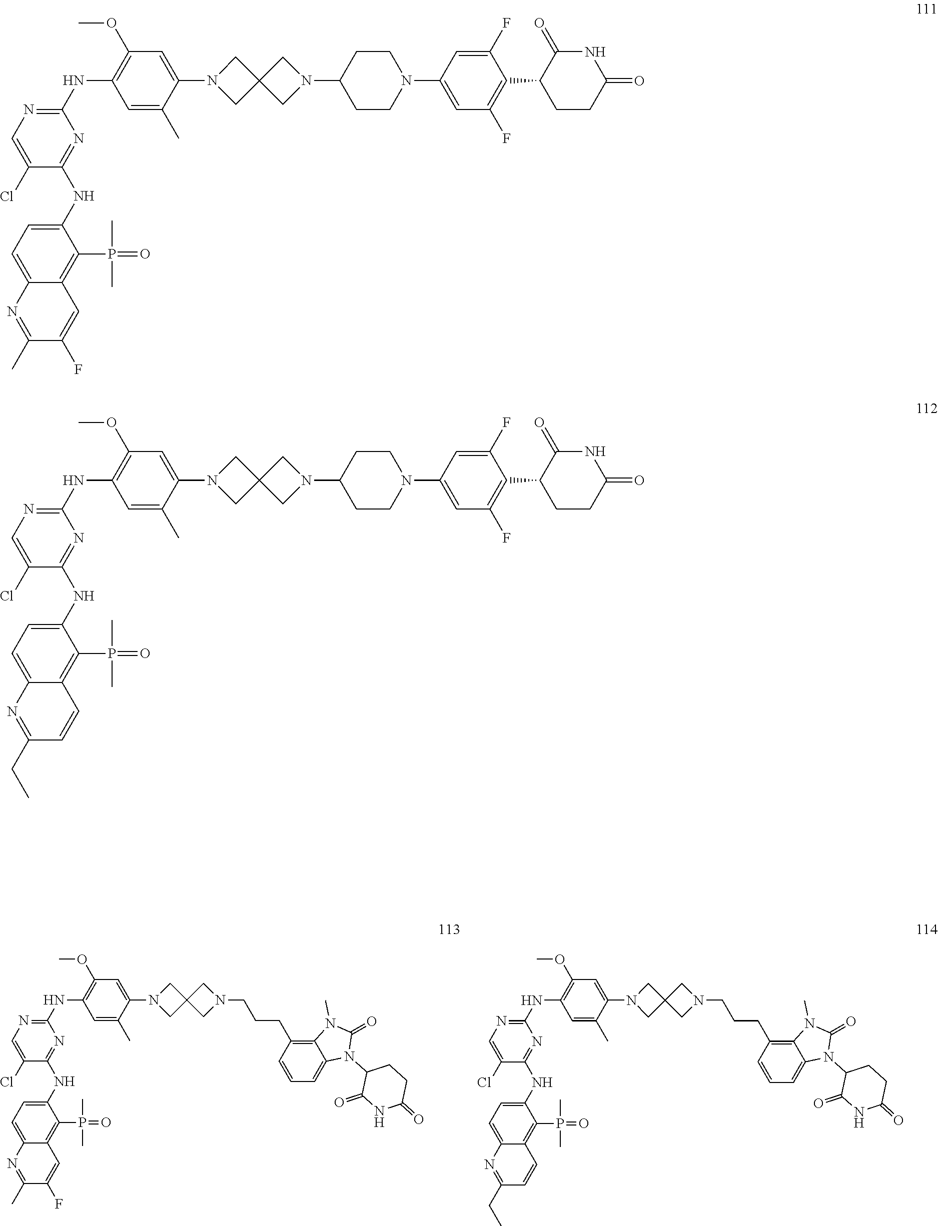 [SMILES: COc1cc(N2CC3(C2)CN(C2CCN(c4cc(F)c([C@H]5CCC(=O)NC5=O)c(F)c4)CC2)C3)c(C)cc1Nc1ncc(Cl)c(Nc2ccc3nc(C)c(F)cc3c2P(C)(C)=O)n1 ; CCc1ccc2c(P(C)(C)=O)c(Nc3nc(Nc4cc(C)c(N5CC6(C5)CN(C5CCN(c7cc(F)c([C@H]8CCC(=O)NC8=O)c(F)c7)CC5)C6)cc4OC)ncc3Cl)ccc2n1 ; COc1cc(N2CC3(CN(CCCc4cccc5c4n(C)c(=O)n5C4CCC(=O)NC4=O)C3)C2)c(C)cc1Nc1ncc(Cl)c(Nc2ccc3nc(C)c(F)cc3c2P(C)(C)=O)n1 ; CCc1ccc2c(P(C)(C)=O)c(Nc3nc(Nc4cc(C)c(N5CC6(CN(CCCc7cccc8c7n(C)c(=O)n8C7CCC(=O)NC7=O)C6)C5)cc4OC)ncc3Cl)ccc2n1]

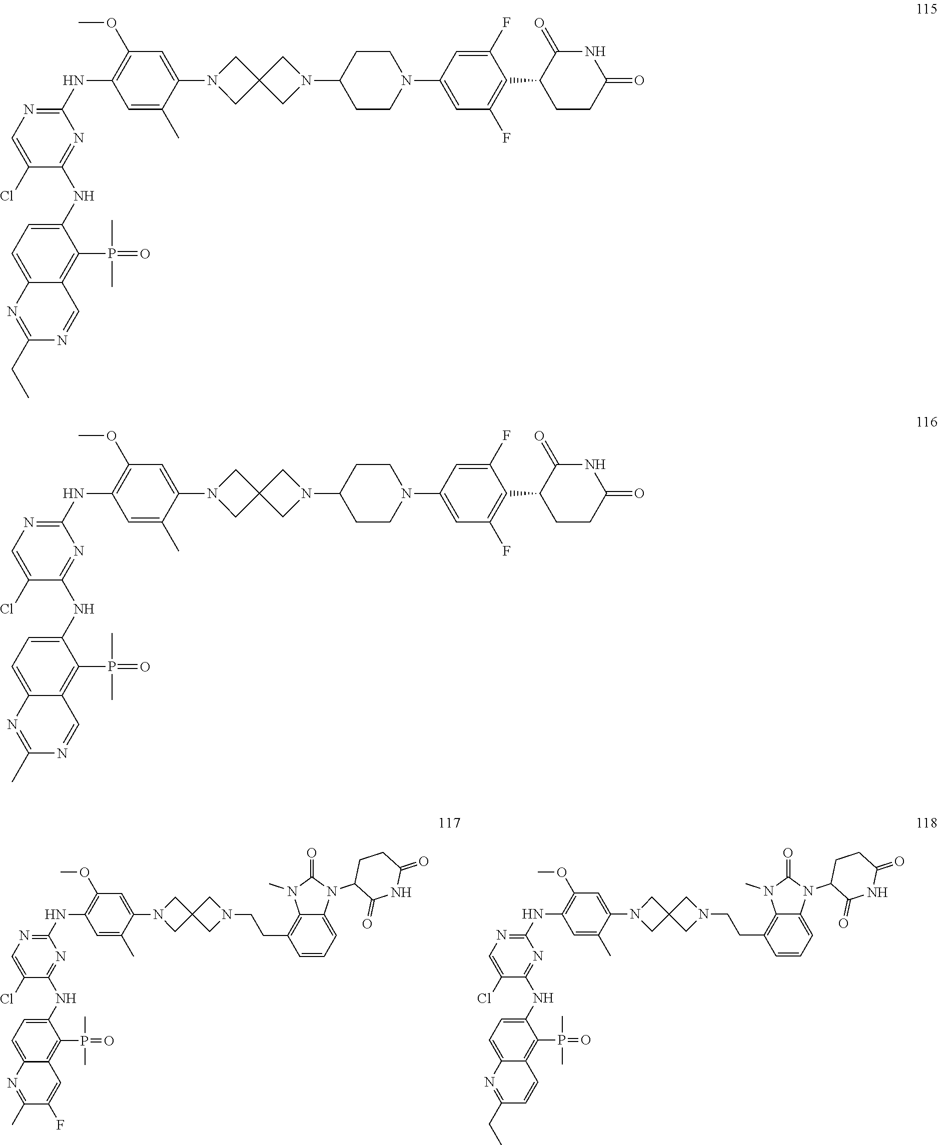 [SMILES: CCc1ncc2c(P(C)(C)=O)c(Nc3nc(Nc4cc(C)c(N5CC6(C5)CN(C5CCN(c7cc(F)c([C@H]8CCC(=O)NC8=O)c(F)c7)CC5)C6)cc4OC)ncc3Cl)ccc2n1 ; COc1cc(N2CC3(C2)CN(C2CCN(c4cc(F)c([C@H]5CCC(=O)NC5=O)c(F)c4)CC2)C3)c(C)cc1Nc1ncc(Cl)c(Nc2ccc3nc(C)ncc3c2P(C)(C)=O)n1 ; COc1cc(N2CC3(CN(CCc4cccc5c4n(C)c(=O)n5C4CCC(=O)NC4=O)C3)C2)c(C)cc1Nc1ncc(Cl)c(Nc2ccc3nc(C)c(F)cc3c2P(C)(C)=O)n1 ; CCc1ccc2c(P(C)(C)=O)c(Nc3nc(Nc4cc(C)c(N5CC6(CN(CCc7cccc8c7n(C)c(=O)n8C7CCC(=O)NC7=O)C6)C5)cc4OC)ncc3Cl)ccc2n1]

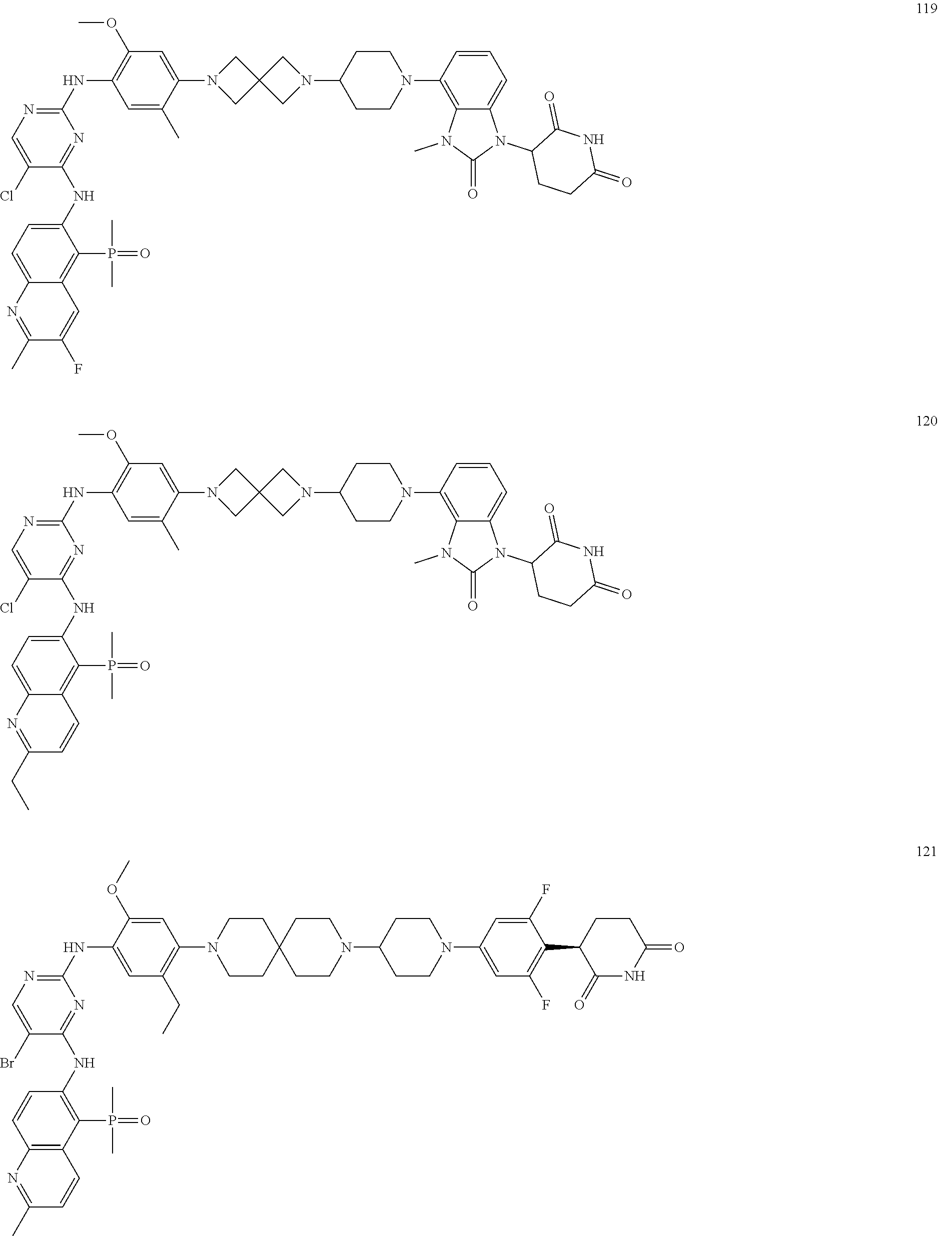 [SMILES: COc1cc(N2CC3(C2)CN(C2CCN(c4cccc5c4n(C)c(=O)n5C4CCC(=O)NC4=O)CC2)C3)c(C)cc1Nc1ncc(Cl)c(Nc2ccc3nc(C)c(F)cc3c2P(C)(C)=O)n1 ; CCc1ccc2c(P(C)(C)=O)c(Nc3nc(Nc4cc(C)c(N5CC6(C5)CN(C5CCN(c7cccc8c7n(C)c(=O)n8C7CCC(=O)NC7=O)CC5)C6)cc4OC)ncc3Cl)ccc2n1 ; CCc1cc(Nc2ncc(Br)c(Nc3ccc4nc(C)ccc4c3P(C)(C)=O)n2)c(OC)cc1N1CCC2(CC1)CCN(C1CCN(c3cc(F)c([C@H]4CCC(=O)NC4=O)c(F)c3)CC1)CC2]

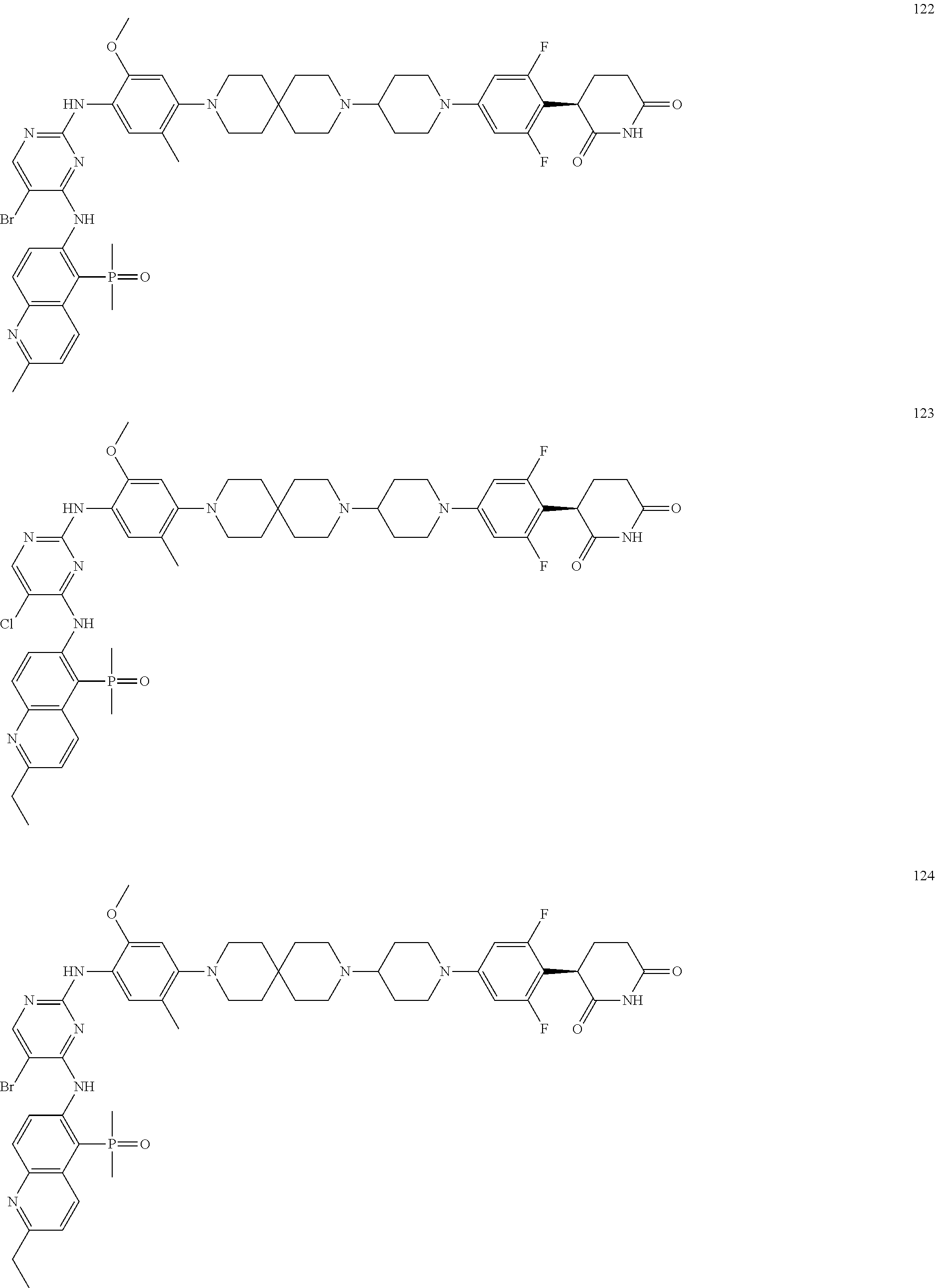 [SMILES: COc1cc(N2CCC3(CC2)CCN(C2CCN(c4cc(F)c([C@H]5CCC(=O)NC5=O)c(F)c4)CC2)CC3)c(C)cc1Nc1ncc(Br)c(Nc2ccc3nc(C)ccc3c2P(C)(C)=O)n1 ; CCc1ccc2c(P(C)(C)=O)c(Nc3nc(Nc4cc(C)c(N5CCC6(CC5)CCN(C5CCN(c7cc(F)c([C@H]8CCC(=O)NC8=O)c(F)c7)CC5)CC6)cc4OC)ncc3Cl)ccc2n1 ; CCc1ccc2c(P(C)(C)=O)c(Nc3nc(Nc4cc(C)c(N5CCC6(CC5)CCN(C5CCN(c7cc(F)c([C@H]8CCC(=O)NC8=O)c(F)c7)CC5)CC6)cc4OC)ncc3Br)ccc2n1]

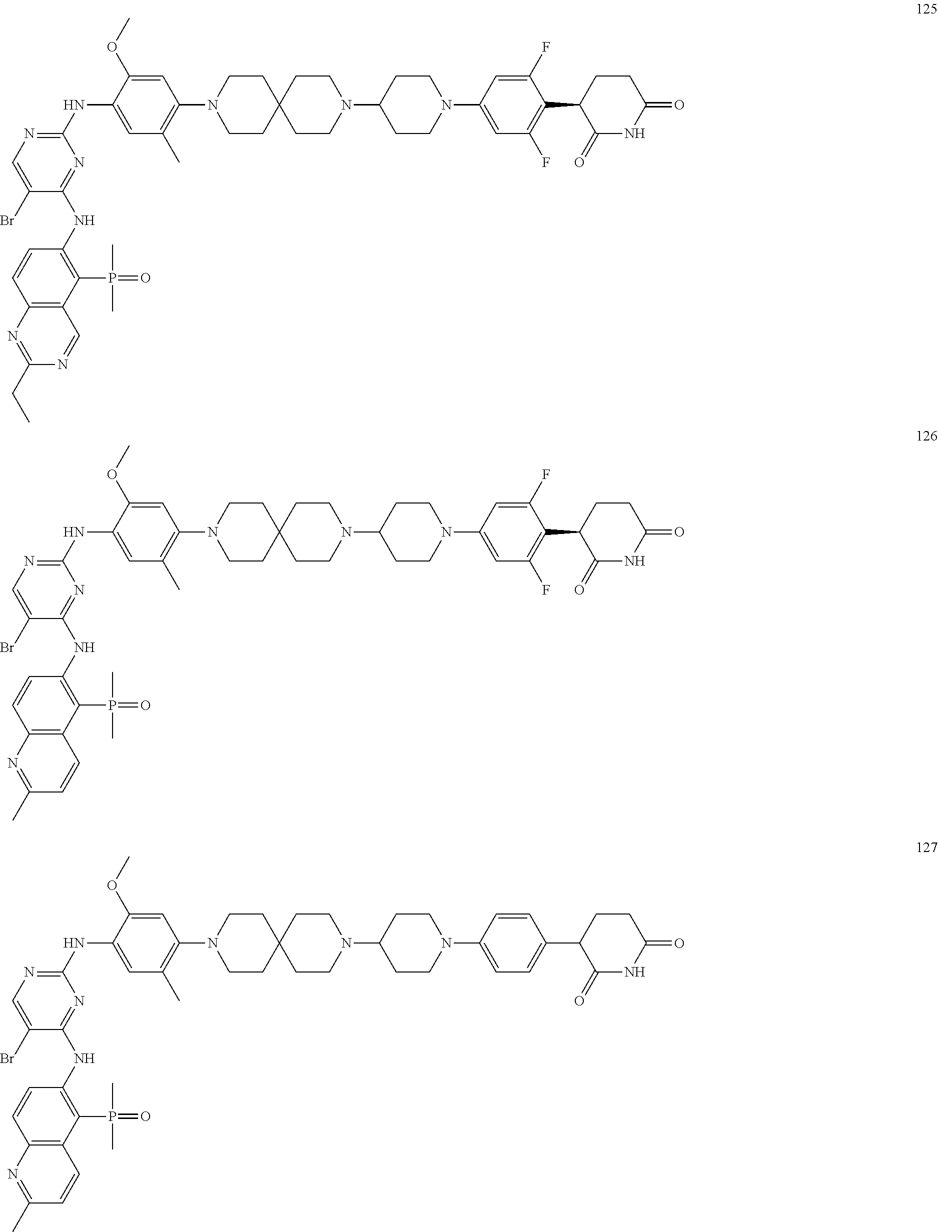 [SMILES: CCc1ncc2c(P(C)(C)=O)c(Nc3nc(Nc4cc(C)c(N5CCC6(CC5)CCN(C5CCN(c7cc(F)c([C@H]8CCC(=O)NC8=O)c(F)c7)CC5)CC6)cc4OC)ncc3Br)ccc2n1 ; COc1cc(N2CCC3(CC2)CCN(C2CCN(c4cc(F)c([C@H]5CCC(=O)NC5=O)c(F)c4)CC2)CC3)c(C)cc1Nc1ncc(Br)c(Nc2ccc3nc(C)ccc3c2P(C)(C)=O)n1 ; COc1cc(N2CCC3(CC2)CCN(C2CCN(c4ccc(C5CCC(=O)NC5=O)cc4)CC2)CC3)c(C)cc1Nc1ncc(Br)c(Nc2ccc3nc(C)ccc3c2P(C)(C)=O)n1]

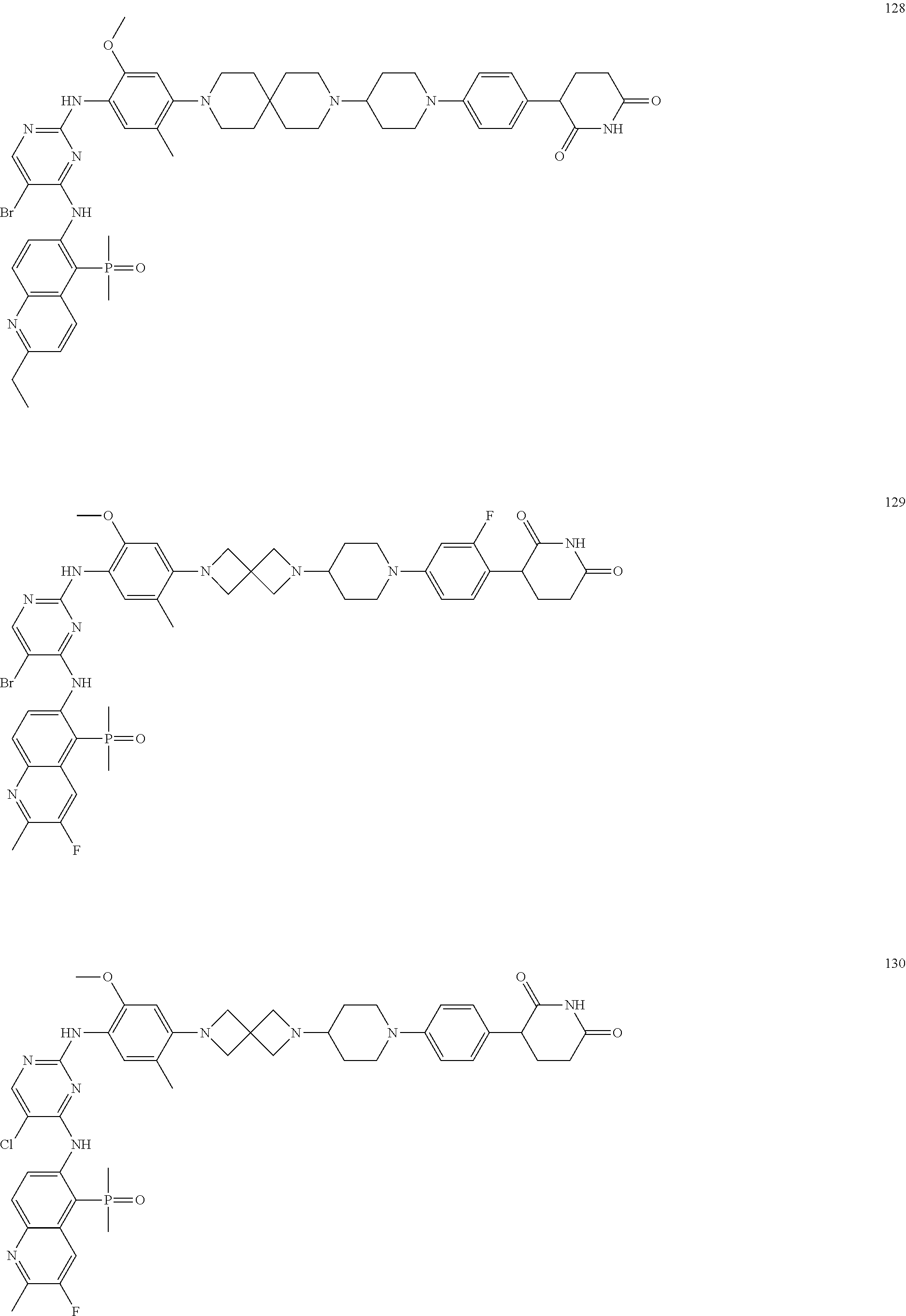 [SMILES: CCc1ccc2c(P(C)(C)=O)c(Nc3nc(Nc4cc(C)c(N5CCC6(CC5)CCN(C5CCN(c7ccc(C8CCC(=O)NC8=O)cc7)CC5)CC6)cc4OC)ncc3Br)ccc2n1 ; COc1cc(N2CC3(C2)CN(C2CCN(c4ccc(C5CCC(=O)NC5=O)c(F)c4)CC2)C3)c(C)cc1Nc1ncc(Br)c(Nc2ccc3nc(C)c(F)cc3c2P(C)(C)=O)n1 ; COc1cc(N2CC3(C2)CN(C2CCN(c4ccc(C5CCC(=O)NC5=O)cc4)CC2)C3)c(C)cc1Nc1ncc(Cl)c(Nc2ccc3nc(C)c(F)cc3c2P(C)(C)=O)n1]

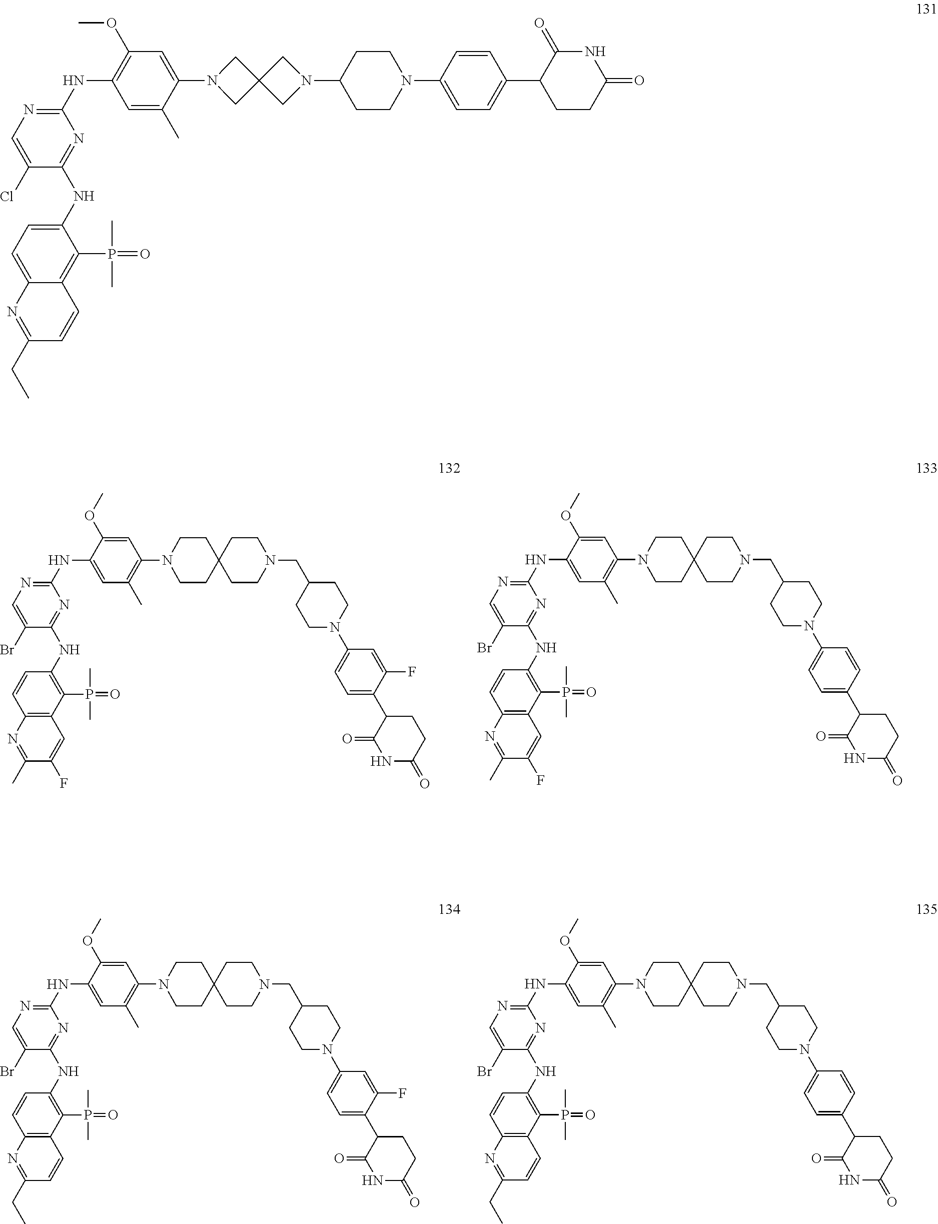

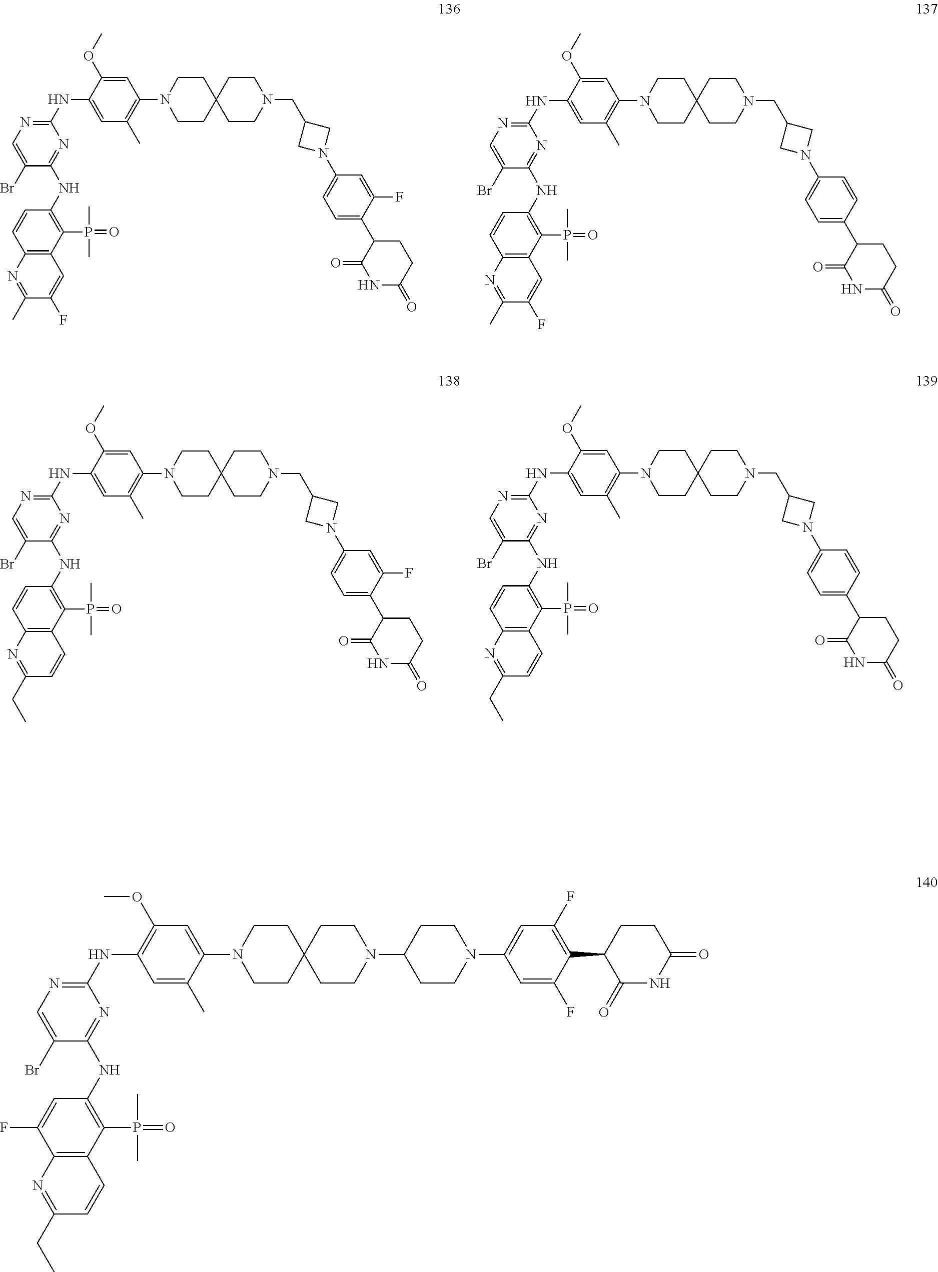

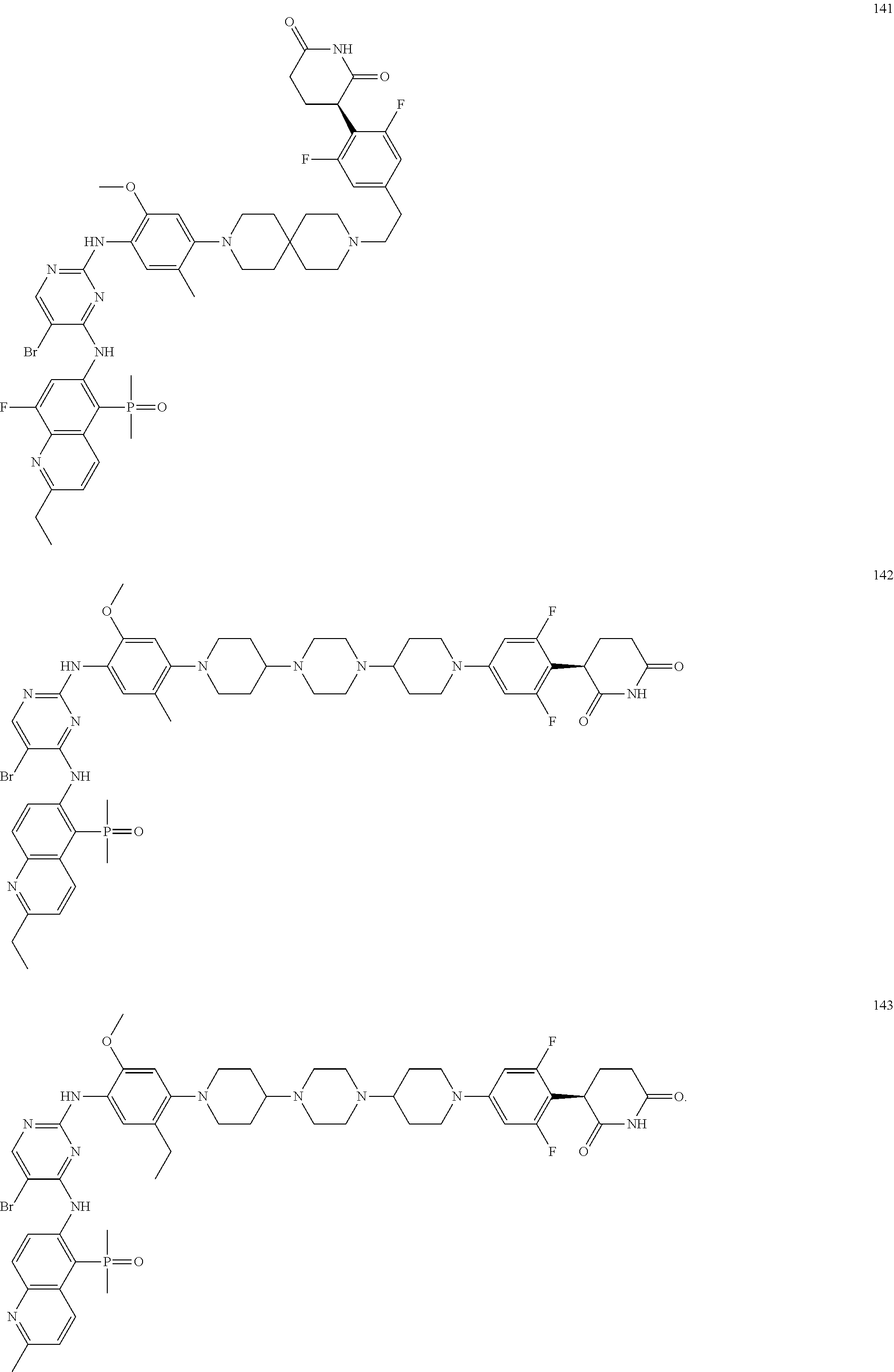 [SMILES: CCc1ccc2c(P(C)(C)=O)c(Nc3nc(Nc4cc(C)c(N5CCC6(CCN(CCc7cc(F)c([C@H]8CCC(=O)NC8=O)c(F)c7)CC6)CC5)cc4OC)ncc3Br)cc(F)c2n1 ; CCc1ccc2c(P(C)(C)=O)c(Nc3nc(Nc4cc(C)c(N5CCC(N6CCN(C7CCN(c8cc(F)c([C@H]9CCC(=O)NC9=O)c(F)c8)CC7)CC6)CC5)cc4OC)ncc3Br)ccc2n1 ; CCc1cc(Nc2ncc(Br)c(Nc3ccc4nc(C)ccc4c3P(C)(C)=O)n2)c(OC)cc1N1CCC(N2CCN(C3CCN(c4cc(F)c([C@H]5CCC(=O)NC5=O)c(F)c4)CC3)CC2)CC1]

In some embodiments, the compounds disclosed herein, including the general compounds and the specific compounds, are not any one of the compounds of Examples 1-756 of PCT/CN2021/106482.

Aspect 24. A pharmaceutical composition comprising a compound of any one of Aspects 1-23 or a pharmaceutically acceptable salt, stereoisomer, tautomer or prodrug thereof, together with a pharmaceutically acceptable excipient.

Aspect 25. A method of treating a disease that can be affected by EGFR modulation, comprises administrating a subject in need thereof an effective amount of a compound of any one of Aspects 1-23 or a pharmaceutically acceptable salt, stereoisomer, tautomer or prodrug thereof.

Aspect 26. The method of Aspect 25, wherein the disease is selected from cancer, preferred pancreatic cancer, breast cancer, glioblastoma multiforme, head and neck cancer, or non-small cell lung cancer.

Aspect 27. Use of a compound of any one of Aspects 1-23 or a pharmaceutically acceptable salt, stereoisomer, tautomer or prodrug thereof in the preparation of a medicament for treating a disease that can be affected by EGFR modulation.

Aspect 28. The use of Aspect 27, wherein the disease is cancer, preferred pancreatic cancer, breast cancer, glioblastoma multiforme, head and neck cancer, or non-small cell lung cancer.

### DETAILED DESCRIPTION OF THE INVENTION

The following terms have the indicated meanings throughout the specification:

Unless specifically defined elsewhere in this document, all other technical and scientific terms used herein have the meaning commonly understood by one of ordinary skill in the art to which this invention belongs.

The following terms have the indicated meanings throughout the specification:

As used herein, including the appended claims, the singular forms of words such as “a”, “an”, and “the”, include their corresponding plural references unless the context clearly indicates otherwise.

The term “or” is used to mean, and is used interchangeably with, the term “and/or” unless the context clearly dictates otherwise.

The term “alkyl” includes a hydrocarbon group selected from linear and branched, saturated hydrocarbon groups comprising from 1 to 18, such as from 1 to 12, further such as from 1 to 10, more further such as from 1 to 8, or from 1 to 6, or from 1 to 4, carbon atoms. Examples of alkyl groups comprising from 1 to 6 carbon atoms (i.e., C₁₋₆ alkyl) include, but not limited to, methyl, ethyl, 1-propyl or n-propyl (“n-Pr”), 2-propyl or isopropyl (“i-Pr”), 1-butyl or n-butyl (“n-Bu”), 2-methyl-1-propyl or isobutyl (“i-Bu”), 1-methylpropyl or s-butyl (“s-Bu”), 1,1-dimethylethyl ort-butyl (“t-Bu”), 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl and 3,3-dimethyl-2-butyl groups.

The term “propyl” canes 1-propyl or n-propyl or n-Pr, 2-propyl or isopropyl or i-Pr.

The term “butyl” includes 1-butyl or n-butyl or n-Bu, 2-methyl-1-propyl or isobutyl or i-Bu, 1-methylpropyl or s-butyl or s-Bu, 1,1-dimethylethyl or t-butyl or t-Bu.

The term “pentyl” includes 1-pentyl, 2-pentyl, 3-pentyl, 2-methyl-2-butyl, 3-methyl-2-butyl, 3-methyl-1-butyl, 2-methyl-1-butyl.

The term “hexyl” includes 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl and 3,3-dimethyl-2-butyl.

The term “alkylene” refers to a divalent alkyl group by removing two hydrogen from alkane. Alkylene includes but not limited to methylene, ethylene, propylene, and so on.

The term “halogen” includes fluoro (F), chloro (Cl), bromo (Br) and iodo (I).

The term “alkenyl” includes a hydrocarbon group selected from linear and branched hydrocarbon groups comprising at least one C═C double bond and from 2 to 18, such as from 2 to 8, further such as from 2 to 6, carbon atoms. Examples of the alkenyl group, e.g., C₂₋₆ alkenyl, include, but not limited to ethenyl or vinyl, prop-1-enyl, prop-2-enyl, 2-methylprop-1-enyl, but-1-enyl, but-2-enyl, but-3-enyl, buta-1,3-dienyl, 2-methylbuta-1,3-dienyl, hex-1-enyl, hex-2-enyl, hex-3-enyl, hex-4-enyl, and hexa-1,3-dienyl groups.

The term “alkenylene” refers to a divalent alkenyl group by removing two hydrogen from alkene. Alkenylene includes but not limited to, vinylidene, butenylene, and so on.

The term “alkynyl” includes a hydrocarbon group selected from linear and branched hydrocarbon group, comprising at least one C≡C triple bond and from 2 to 18, such as 2 to 8, further such as from 2 to 6, carbon atoms. Examples of the alkynyl group, e.g., C₂₋₆ alkynyl, include, but not limited to ethynyl, 1-propynyl, 2-propynyl (propargyl), 1-butynyl, 2-butynyl, and 3-butynyl groups.

The term “alkynylene” refers to a divalent alkynyl group by removing two hydrogen from alkyne. Alkynylene includes but not limited to ethynylene and so on.

The term “cycloalkyl” includes a hydrocarbon group selected from saturated cyclic hydrocarbon groups, comprising monocyclic and polycyclic (e.g., bicyclic and tricyclic) groups including fused, bridged or spiro cycloalkyl.

For example, the cycloalkyl group may comprise from 3 to 12, such as from 3 to 10, further such as 3 to 8, further such as 3 to 6, 3 to 5, or 3 to 4 carbon atoms. Even further for example, the cycloalkyl group may be selected from monocyclic group comprising from 3 to 12, such as from 3 to 10, further such as 3 to 8, 3 to 6 carbon atoms. Examples of the monocyclic cycloalkyl group include cyclopropyl, cyclobutyl, cyclopentyl, cyclohexyl, cycloheptyl, cyclooctyl, cyclononyl, cyclodecyl, cycloundecyl, and cyclododecyl groups. In particular, examples of the saturated monocyclic cycloalkyl group, e.g., C₃₋₈cycloalkyl, include, but not limited to cyclopropyl, cyclobutyl, cyclopentyl, cyclohexyl, cycloheptyl, and cyclooctyl groups. In a preferred embodiment, the cycloalkyl is a monocyclic ring comprising 3 to 6 carbon atoms (abbreviated as C₃₋₆ cycloalkyl), including but not limited to, cyclopropyl, cyclobutyl, cyclopentyl, and cyclohexyl. Examples of the bicyclic cycloalkyl groups include those having from 7 to 12 ring atoms arranged as a fused bicyclic ring selected from [4,4], [4,5], [5,5], [5,6] and [6,6] ring systems, or as a bridged bicyclic ring selected from bicyclo[2.2.1]heptane, bicyclo[2.2.2]octane, and bicyclo[3.2.2]nonane. Further Examples of the bicyclic cycloalkyl groups include those arranged as a bicyclic ring selected from [5,6] and [6,6] ring systems.

The term “spiro cycloalkyl” includes a cyclic structure which contains carbon atoms and is formed by at least two rings sharing one atom.

The term “fused cycloalkyl” includes a bicyclic cycloalkyl group as defined herein which is saturated and is formed by two or more rings sharing two adjacent atoms.

The term “bridged cycloalkyl” includes a cyclic structure which contains carbon atoms and is formed by two rings sharing two atoms which are not adjacent to each other. The term “7 to 10 membered bridged cycloalkyl” includes a cyclic structure which contains 7 to 12 carbon atoms and is formed by two rings sharing two atoms which are not adjacent to each other.

Examples of fused cycloalkyl, fused cycloalkenyl, or fused cycloalkynyl include but are not limited to bicyclo[1.1.0]butyl, bicyclo[2.1.0]pentyl, bicyclo[3.1.0]hexyl, bicyclo[4.1.0]heptyl, bicyclo[3.3.0]octyl, bicyclo[4.2.0]octyl, decalin, as well as benzo 3 to 8 membered cycloalkyl, benzo C₄0.6 cycloalkenyl, 2,3-dihydro-1H-indenyl, 1H-indenyl, 1, 2, 3,4-tetralyl, 1,4-dihydronaphthyl, etc. Preferred embodiments are 8 to 9 membered fused rings, which refer to cyclic structures containing 8 to 9 ring atoms within the above examples.

The term “aryl” used alone or in combination with other terms includes a group selected from:
5- and 6-membered carbocyclic aromatic rings, e.g., phenyl;
bicyclic ring systems such as 7 to 12 membered bicyclic ring systems, wherein at least one ring is carbocyclic and aromatic, e.g., naphthyl and indanyl; and,
tricyclic ring systems such as 10 to 15 membered tricyclic ring systems wherein at least one ring is carbocyclic and aromatic, e.g., fluorenyl.

The terms “aromatic hydrocarbon ring” and “aryl” are used interchangeably throughout the disclosure herein. In some embodiments, a monocyclic or bicyclic aromatic hydrocarbon ring has 5 to 10 ring-forming carbon atoms (i.e., C₅₋₁₀ aryl). Examples of a monocyclic or bicyclic aromatic hydrocarbon ring include, but not limited to, phenyl, naphth-1-yl, naphth-2-yl, anthracenyl, phenanthrenyl, and the like. In some embodiments, the aromatic hydrocarbon ring is a naphthalene ring (naphth-1-yl or naphth-2-yl) or phenyl ring. In some embodiments, the aromatic hydrocarbon ring is a phenyl ring.

Specifically, the term “bicyclic fused aryl” includes a bicyclic aryl ring as defined herein. The typical bicyclic fused aryl is naphthalene.

The term “heteroaryl” includes a group selected from:
5-, 6- or 7-membered aromatic, monocyclic rings comprising at least one heteroatom, for example, from 1 to 4, or, in some embodiments, from 1 to 3, in some embodiments, from 1 to 2, heteroatoms, selected from nitrogen (N), sulfur (S) and oxygen (O), with the remaining ring atoms being carbon;
7- to 12-membered bicyclic rings comprising at least one heteroatom, for example, from 1 to 4, or, in some embodiments, from 1 to 3, or, in other embodiments, 1 or 2, heteroatoms, selected from N, O, and S, with the remaining ring atoms being carbon and wherein at least one ring is aromatic and at least one heteroatom is present in the aromatic ring; and
11- to 14-membered tricyclic rings comprising at least one heteroatom, for example, from 1 to 4, or in some embodiments, from 1 to 3, or, in other embodiments, 1 or 2, heteroatoms, selected from N, O, and S, with the remaining ring atoms being carbon and wherein at least one ring is aromatic and at least one heteroatom is present in an aromatic ring.

When the total number of S and O atoms in the heteroaryl group exceeds 1, those heteroatoms are not adjacent to one another. In some embodiments, the total number of S and O atoms in the heteroaryl group is not more than 2. In some embodiments, the total number of S and O atoms in the aromatic heterocycle is not more than 1. When the heteroaryl group contains more than one heteroatom ring member, the heteroatoms may be the same or different. The nitrogen atoms in the ring(s) of the heteroaryl group can be oxidized to form N-oxides.

Specifically, the term “bicyclic fused heteroaryl” includes a 7- to 12-membered, preferably 7- to 10-membered, more preferably 9- or 10-membered fused bicyclic heteroaryl ring as defined herein. Typically, a bicyclic fused heteroaryl is 5-membered/5-membered, 5-membered/6-membered, 6-membered/6-membered, or 6-membered/7-membered bicyclic. The group can be attached to the remainder of the molecule through either ring.

“Heterocyclyl”, “heterocycle” or “heterocyclic” are interchangeable and include a non-aromatic heterocyclyl group comprising one or more heteroatoms selected from nitrogen, oxygen or optionally oxidized sulfur as ring members, with the remaining ring members being carbon, including monocyclic, fused, bridged, and spiro ring, i.e., containing monocyclic heterocyclyl, bridged heterocyclyl, spiro heterocyclyl, and fused heterocyclic groups.

The term “H” or “hydrogen” disclosed herein includes Hydrogen and the non-radioisotope deuterium.

The term “at least one substituent” disclosed herein includes, for example, from 1 to 4, such as from 1 to 3, further as 1 or 2, substituents, provided that the theory of valence is met. For example, “at least one substituent F” disclosed herein includes from 1 to 4, such as from 1 to 3, further as 1 or 2, substituents F.

The term “divalent” refers to a linking group capable of forming covalent bonds with two other moieties. For example, “a divalent cycloalkyl group” refers to a cycloalkyl group obtained by removing two hydrogen from the corresponding cycloalkane to form a linking group. the term “divalent aryl group”, “divalent heterocyclyl group” or “divalent heteroaryl group” should be understood in a similar manner.

Compounds disclosed herein may contain an asymmetric center and may thus exist as enantiomers. “Enantiomers” refer to two stereoisomers of a compound which are non-superimposable mirror images of one another. Where the compounds disclosed herein possess two or more asymmetric centers, they may additionally exist as diastereomers. Enantiomers and diastereomers fall within the broader class of stereoisomers. All such possible stereoisomers as substantially pure resolved enantiomers, racemic mixtures thereof, as well as mixtures of diastereomers are intended to be included. All stereoisomers of the compounds disclosed herein and/or pharmaceutically acceptable salts thereof are intended to be included. Unless specifically mentioned otherwise, reference to one isomer applies to any of the possible isomers. Whenever the isomeric composition is unspecified, all possible isomers are included.

When compounds disclosed herein contain olefinic double bonds, unless specified otherwise, such double bonds are meant to include both E and Z geometric isomers.

When compounds disclosed herein contain a di-substituted cyclic ring system, substituents found on such ring system may adopt cis and trans formations. Cis formation means that both substituents are found on the upper side of the 2 substituent placements on the carbon, while trans would mean that they were on opposing sides. For example, the di-substituted cyclic ring system may be cyclohexyl or cyclobutyl ring.

It may be advantageous to separate reaction products from one another and/or from starting materials. The desired products of each step or series of steps is separated and/or purified (hereinafter separated) to the desired degree of homogeneity by the techniques common in the art. Typically such separations involve multiphase extraction, crystallization from a solvent or solvent mixture, distillation, sublimation, or chromatography. Chromatography can involve any number of methods including, for example: reverse-phase and normal phase; size exclusion; ion exchange; high, medium and low pressure liquid chromatography methods and apparatus; small scale analytical; simulated moving bed (“SMB”) and preparative thin or thick layer chromatography, as well as techniques of small scale thin layer and flash chromatography. One skilled in the art could select and apply the techniques most likely to achieve the desired separation.

“Diastereomers” refer to stereoisomers of a compound with two or more chiral centers but which are not mirror images of one another. Diastereomeric mixtures can be separated into their individual diastereomers on the basis of their physical chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereoisomers to the corresponding pure enantiomers. Enantiomers can also be separated by use of a chiral HPLC column.

A single stereoisomer, e.g., a substantially pure enantiomer, may be obtained by resolution of the racemic mixture using a method such as formation of diastereomers using optically active resolving agents (Eliel, E. and Wilen, S. Stereochemistry of Organic Compounds. New York: John Wiley & Sons, Inc., 1994; Lochmuller, C. H, et al. “Chromatographic resolution of enantiomers: Selective “eview.” J Chromatogr., 113(3) (1975): pp. 283-302). Racemic mixtures of chiral compounds of the invention can be separated and isolated by any suitable method, including: (1) formation of ionic, diastereomeric salts with chiral compounds and separation by fractional crystallization or other methods, (2) formation of diastereomeric compounds with chiral derivatizing reagents, separation of the diastereomers, and conversion to the pure stereoisomers, and (3) separation of the substantially pure or enriched stereoisomers directly under chiral conditions. See: Wainer, Irving W., Ed. Drug Stereochemistry: Analytical Methods and Pharmacology. New York: Marcel Dekker, Inc., 1993.

Some of the compounds disclosed herein may exist with different points of attachment of hydrogen, referred to as tautomers. For example, compounds including carbonyl —CH₂C(O)— groups (keto forms) may undergo tautomerism to form hydroxyl —CH═C(OH)— groups (enol forms). Both keto and enol forms, individually as well as mixtures thereof, are also intended to be included where applicable.

“Prodrug” refers to a derivative of an active agent that requires a transformation within the body to release the active agent. In some embodiments, the transformation is an enzymatic transformation. Prodrugs are frequently, although not necessarily, pharmacologically inactive until converted to the active agent.

“deuterated analog” refers to a derivative of an active agent that an arbitrary hydrogen is substituted with deuterium. In some embodiments, the deuterated site is on the Warhead moiety. In some embodiments, the deuterated site is on the Linker moiety. In some embodiments, the deuterated site is on the Degron moiety.

“Pharmaceutically acceptable salts” refer to those salts which are, within the scope of sound medical judgment, suitable for use in contact with the tissues of humans and lower animals without undue toxicity, irritation, allergic response and the like, and are commensurate with a reasonable benefit/risk ratio. A pharmaceutically acceptable salt may be prepared in situ during the final isolation and purification of the compounds disclosed herein, or separately by reacting the free base function with a suitable organic acid or by reacting the acidic group with a suitable base. The term also includes salts of the stereoisomers (such as enantiomers and/or diastereomers), tautomers and prodrugs of the compound of the invention.

In addition, if a compound disclosed herein is obtained as an acid addition salt, the free base can be obtained by basifying a solution of the acid salt. Conversely, if the product is a free base, an addition salt, such as a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used without undue experimentation to prepare non-toxic pharmaceutically acceptable addition salts.

The terms “administration”, “administering”, “treating” and “treatment” herein, when applied to an animal, human, experimental subject, cell, tissue, organ, or biological fluid, mean contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell. The term “administration” and “treatment” also means in vitro and ex vivo treatments, e.g., of a cell, by a reagent, diagnostic, binding compound, or by another cell. The term “subject” herein includes any organism, preferably an animal, more preferably a mammal (e.g., rat, mouse, dog, cat, and rabbit) and most preferably a human.

The term “effective amount” or “therapeutically effective amount” refers to an amount of the active ingredient, such as a compound that, when administered to a subject for treating a disease, or at least one of the clinical symptoms of a disease or disorder, is sufficient to affect such treatment for the disease, disorder, or symptom. The term “therapeutically effective amount” can vary with the compound, the disease, disorder, and/or symptoms of the disease or disorder, severity of the disease, disorder, and/or symptoms of the disease or disorder, the age of the subject to be treated, and/or the weight of the subject to be treated. An appropriate amount in any given instance can be apparent to those skilled in the art or can be determined by routine experiments. In some embodiments, “therapeutically effective amount” is an amount of at least one compound and/or at least one stereoisomer, tautomer or prodrug thereof, and/or at least one pharmaceutically acceptable salt thereof disclosed herein effective to “treat” as defined herein, a disease or disorder in a subject. In the case of combination therapy, the term “therapeutically effective amount” refers to the total amount of the combination objects for the effective treatment of a disease, a disorder or a condition.

The term “disease” refers to any disease, discomfort, illness, symptoms or indications, and can be interchangeable with the term “disorder” or “condition”.

Throughout this specification and the claims which follow, unless the context requires otherwise, the term “comprise”, and variations such as “comprise” and “comprising” are intended to specify the presence of the features thereafter, but do not exclude the presence or addition of one or more other features. When used herein the term “comprising” can be substituted with the term “containing”, “including” or sometimes “having”.

Throughout this specification and the claims which follow, the term “Cₙ₋ₘ” or “Cₙ-Cₘ” indicates a range which includes the endpoints, wherein n and m are integers and indicate the number of carbons. Examples include C₁₋₈, C₁₋₆, C₁-C₈, C₁-C₆ and the like.

Unless specifically defined elsewhere in this document, all other technical and scientific terms used herein have the meaning commonly understood by one of ordinary skill in the art to which this invention belongs.

### EXAMPLES

The examples below are intended to be purely exemplary and should not be considered to be limiting in any way. Efforts have been made to ensure accuracy with respect to numbers used (for example, amounts, temperature, etc.), but some experimental errors and deviations should be accounted for. Unless indicated otherwise, temperature is in degrees Centigrade. Reagents were purchased from commercial suppliers such as Sigma-Aldrich, Alfa Aesar, or TCI, and were used without further purification unless indicated otherwise.

Unless indicated otherwise, the reactions set forth below were performed under a positive pressure of nitrogen or argon or with a drying tube in anhydrous solvents; the reaction flasks were fitted with rubber septa for the introduction of substrates and reagents via syringe; and glassware was oven dried and/or heat dried.

¹H NMR spectra were recorded on an Agilent instrument operating at 400 MHz. ¹HNMR

spectra were obtained using CDCl₃, CD₂Cl₂, CD₃OD, D₂O, d₆-DMSO, d₆-acetone or (CD₃)₂CO as solvent and tetramethylsilane (0.00 ppm) or residual solvent (CDCl₃: 7.25 ppm; CD₃OD: 3.31 ppm; D₂O: 4.79 ppm; d₆-DMSO: 2.50 ppm; d₆-acetone: 2.05; (CD₃)₃CO: 2.05) as the reference standard. When peak multiplicities are reported, the following abbreviations are used: s (singlet), d (doublet), t (triplet), q (quartet), qn (quintuplet), sx (sextuplet), m (multiplet), br (broadened), dd (doublet of doublets), dt (doublet of triplets). Coupling constants, when given, are reported in Hertz (Hz).

LCMS-1: LC-MS spectrometer (Agilent 1260 Infinity) Detector: MWD (190-400 nm), Mass detector: 6120 SQ Mobile phase: A: water with 0.1% Formic acid, B: acetonitrile with 0.1% Formic acid Column: Poroshell 120 EC-C18, 4.6×50 mm, 2.7 pm Gradient method: Flow: 1.8 mL/min Time (min) A (%) B (%)

| Time (min) | A(%) | B(%) |
|---|---|---|
| 0.00 | 95 | 5 |
| 1.5 | 5 | 95 |
| 2.0 | 5 | 95 |
| 2.1 | 95 | 5 |
| 3.0 | 95 | 5 |

LCMS, LCMS-3: LC-MS spectrometer (Agilent 1260 Infinity II) Detector: MWD (190-400 nm), Mass detector: G6125C SQ Mobile phase: A: water with 0.1% Formic acid, B: acetonitrile with 0.1% Formic acid Column: Poroshell 120 EC-C18, 4.6×50 mm, 2.7 pm Gradient method: Flow: 1.8 mL/min Time (min) A (%) B (%)

| Time (min) | A(%) | B(%) |
|---|---|---|
| 0.00 | 95 | 5 |
| 1.5 | 5 | 95 |
| 2.0 | 5 | 95 |
| 2.1 | 95 | 5 |
| 3.0 | 95 | 5 |

LCMS-2: LC-MS spectrometer (Agilent 1290 Infinity II) Detector: MWD (190-400 nm), Mass detector: G6125C SQ Mobile phase: A: water with 0.1% Formic acid, B: acetonitrile with 0.1 Formic acid Column: Poroshell 120 EC-C18, 4.6×50 mm, 2.7 pm Gradient method Flow: 1.2 mL/min Time (min) A (%) B (%)

| Time (min) | A(%) | B(%) |
|---|---|---|
| 0.00 | 90 | 10 |
| 1.5 | 5 | 95 |
| 2.0 | 5 | 95 |
| 2.1 | 90 | 10 |
| 3.0 | 90 | 10 |

Preparative HPLC was conducted on a column (150×21.2 mm ID, 5 pm, Gemini NXC 18) at a flow rate of 20 ml/min, injection volume 2 ml, at room temperature and UV Detection at 214 nm and 254 nm.

In the following examples, the abbreviations below are used:

| | |
|---|---|
| (BPin)₂ | 4,4,4′,4′,5,5,5′,5′-Octamethyl-2,2′-bi-1,3,2-dioxaborolane |
| Ac₂O | acetic anhydride |
| AcCl | Acetyl chloride |
| ACN or MeCN | Acetonitrile |
| AcOH or HOAc | Acetic acid |
| AcONa or NaOAc | Sodium acetate |
| Aq | Aqueous |
| BINAP | (±)-2,2′-Bis(diphenylphosphino)-1,1′-BINAPhthyl |
| Bn | benzyl |
| BnBr | Benzyl Bromide |
| Boc | t-Butyloxy carbonyl |
| BTEAC | Benzyltriethylammonium chloride |
| C: 40691-33-6 | dichlorobis(tri-o-tolylphosphine)palladium(II) |
| Cbz | Benzyloxycarbonyl |
| DCM | Dichloromethane |
| Con. | Concentrated |
| DavePhos | 2′-(Dicyclohexylphosphino)-N,N-dimethyl-2-biphenylamine |
| DBU | 1,8-diazabicyclo[5.4.0]undec-7-ene |
| DCE | dichloroethane |
| DHP | 3,4-Dihydro-2H-pyran |
| DIBAL-H | Diisobutylaluminium hydride |
| DIEA or DIPEA | N,N-diisopropylethylamine |
| DMAP | 4-N,N-dimethylaminopyridine |
| DMP | Dess-Martin periodinane |
| DMF | N,N-Dimethylformamide |
| DMSO | Dimethyl sulfoxide |
| Dppf | 1,1″-bis(diphenylphosphino)ferrocene |
| EA or EtOAc | Ethyl acetate |
| EtOH | ethanol |
| FA | Formic acid |
| h or hr | Hour |
| HATU | 2-(7-Azabenzotriazol-1-yl)-N,N,N′,N′-tetramethyluronium |
| | hexafluorophosphate |
| HBTU | O-(7-Benzotriazole-1-yl)-N,N,N′,N′-tetramethyluronium |
| | hexafluorophosphate |
| Hex | Hexane |
| HPLC | High Performance Liquid Chromatography |
| hrs | hours |
| IBX | 2-Iodoxybenzoic acid |
| IPA | 2-propanol |
| i-PrOH | Isopropyl alcohol |
| KHMDS | Potassium bis(trimethylsilyl)amide |
| LiHMDS | Lithium bis(trimethylsilyl)amide |
| MeCN or ACN | Acetonitrile |
| MeOH | Methanol |
| Min | Minutes |
| ms or MS | Mass spectrum |
| MsCl | Methanesulfonyl chloride |
| MsOH | Methanesulfonic acid |
| MTBE | Methyl tert-butyl ether |
| o/n | overnight |
| Pd(dppf)Cl₂ | [1,1′-Bis(diphenylphosphino)ferrocene]dichloropalladium(II) |
| Pd₂(dba)₃ | Tris(dibenzylideneacetone)dipalladium |
| PE | Petroleum ether |
| PhMe | Toluene |
| PMB | 4-Methoxybenzyl |
| PPA | Polyphosphoric acid |
| R.T. or r.t. | Room temperature |
| Rt | Retention time |
| Selectfluor | 1-Chloromethyl-4-fluoro-1,4-diazoniabicyclo[2.2.2]octane |
| | bis(tetrafluoroborate) |
| SEMCl | 2-(Trimethylsilyl)ethoxymethyl chloride |
| STAB | Sodium Triacetoxyborohydride; Sodium triacetoborohydride |
| Sat. | saturated |
| SFC | Supercritical Fluid Chromatography |
| TBAF | Tetra-butyl ammonium fluoride |
| TBDPS | tert-Butyldiphenylsilyl |
| TBS | tert-Butyldimethylsilyl |
| TBSCl | tert-Butyldimethylsilyl chloride |
| Ti(OiPr)₄ | Titanium tetraisopropanolate |
| t-Bu | tert-butyl |
| t-BuOH | tert-Butanol |
| t-BuONa | Sodium tert-butoxide |
| t-BuOK | Potassium tert-butoxide |
| TEA | Triethylamine |
| Tf₂O | Triflic anhydride |
| TFA | Trifluoroacetic acid |
| THF | Tetrahydrofuran |
| TLC | Thin layer chromatography |
| TMSOK | Potassium trimethylsilanolate |
| Ts | para-Toluenesulfonyl |
| TsCl | 4-Toluenesulfonyl chloride |
| TsOH | p-toluenesulfonic acid |
| TsOH,Py | Pyridinium toluene-4-sulphonate |
| Xphos or X-phos | 2-Dicyclohexylphosphino-2′,4′,6′-triisopropylbiphenyl |
| XantPhos | 4,5-Bis(diphenylphosphino)-9,9-dimethylxanthene |
| TCFH | N,N,N′,N′-Tetramethylchloroformamidinium hexafluorophosphate |
| CDI | 1,1′-Carbonyldiimidazole |

### Example 1: (R)-3-(4-(4-(9-(4-((5-bromo-4-((5-(dimethylphosphoryl)-3-fluoro-2-methylquinolin-6-yl)amino)pyrimidin-2-yl)amino)-2-ethyl-5-methoxyphenyl)-3,9-diazaspiro[5.5]undecan-3-yl)piperidin-1-yl)-2,6-difluorophenyl)piperidine-2,6-dione

### Step 1: 8-(4-(2,6-bis(benzyloxy)pyridin-3-yl)-3,5-difluorophenyl)-1,4-dioxa-8-azaspiro[4.5]decane

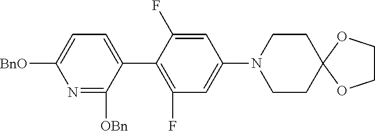 [SMILES: Fc1cc(N2CCC3(CC2)OCCO3)cc(F)c1-c1ccc(OCc2ccccc2)nc1OCc1ccccc1]

To the solution of 2,6-bis(benzyloxy)-3-(4-bromo-2,6-difluorophenyl)pyridine (30 g, 62.24 mmol), 1,4-dioxa-8-azaspiro[4.5]decane (10.68 g, 74.69 mmol) and Cs₂CO₃ (40.58 g, 124.48 mmol) in 500 mL dioxane, Pd₂(dba)₃ (2.85 g, 3.11 mmol) and Xantphos (3.6 g, 6.22 mmol) was added at N₂ atmosphere. The mixture was stirred at 80° C. for 16 hours under N₂ protection. The mixture was diluted with EtOAc and filtered. The filtrated was concentrated in vacuum and purified by silica column chromatography (EA:PE=0-80%) to afford the crude product. The crude was recrystallized with MeOH and filtered. The filter cake was dried to afford the title compound (26.8 g, 79% yield); [M+H]⁺=544.9.

### Step 2: 3-(2,6-difluoro-4-(1,4-dioxa-8-azaspiro[4.5]decan-8-yl)phenyl)piperidine-2,6-dione

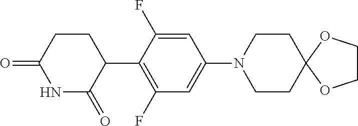 [SMILES: O=C1CCC(c2c(F)cc(N3CCC4(CC3)OCCO4)cc2F)C(=O)N1]

To the solution of 8-(4-(2,6-bis(benzyloxy)pyridin-3-yl)-3,5-difluorophenyl)-1,4-dioxa-8-azaspiro[4.5]decane (26.8 g, 49.26 mmol) in 400 mL DMF and 80 mL iPrOH, Pd/C (27 g, 10 wt. %, wet) was added. The mixture was stirred at 45° C. for 16 hours at H₂ atmosphere (4 bar). The mixture was filtered and the filter cake was washed with DMF. The combined liquid was concentrated in vacuum to afford the title compound (15 g, 83.2% yield); [M+H]⁺=367.1.

### Step 3: (R)-3-(2,6-difluoro-4-(1,4-dioxa-8-azaspiro[4.5]decan-8-yl)phenyl)piperidine-2,6-dione

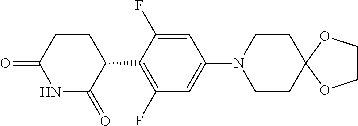 [SMILES: O=C1CC[C@H](c2c(F)cc(N3CCC4(CC3)OCCO4)cc2F)C(=O)N1]

The title compound was purified by SFC (IF (2*25 cm, 5 um), MtBE (0.1% DEA):(MeOH:DCM=1:1)=50:50, 100 bar, 20 ml/min) and the title compound corresponded to peak A @ 0.990 min/254 nm (4.47 g, 37% yield from 12 g racemate); [M+H]⁺=367.1.

### Step 4: (R)-3-(2,6-difluoro-4-(4-oxopiperidin-1-yl)phenyl)piperidine-2,6-dione

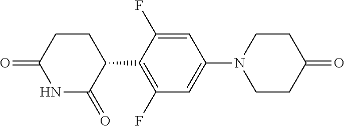 [SMILES: O=C1CCN(c2cc(F)c([C@H]3CCC(=O)NC3=O)c(F)c2)CC1]

(R)-3-(2,6-difluoro-4-(1,4-dioxa-8-azaspiro[4.5]decan-8-yl)phenyl)piperidine-2,6-dione (1 g, 2.73 mmol) was placed in 100 mL round bottom flask with a magnetic stir bar. Then, 10 mL 12N HCl aqueous was added. The mixture was stirred at room temperature for 30 minutes. The mixture was added dropwise to sat. aq. NaHCO₃ solution and finally pH=6-7. The liquid was extracted with DCM and separated. The organic phase was concentrated in vacuum and purified with combiflash (MeOH:DCM=0-5%) to afford the title compound (850 mg, 96.7% yield); [M+H]⁺=323.1.

### Step 5: 1-fluoro-5-methoxy-4-nitro-2-vinylbenzene

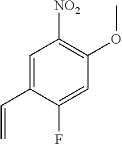

Into a 2 L round-bottom flask were added 1-bromo-2-fluoro-4-methoxy-5-nitrobenzene (40 g, 160.64 mmol), Potassium Vinyltrifluoroborate (43.1 g, 321.28 mmol), Pd(dppf)Cl₂ (11.6 g, 16.06 mmol), and K₂CO₃ (66.5 g, 481.92 mmol) in 1,4-dioxane (400 mL) and H₂O (80 mL). The mixture was stirred at 100° C. for 1.5 h under N₂ protected. The mixture was concentrated in vacuum and purified by silica column chromatography (EA:PE=0-10%) to afford 1-fluoro-5-methoxy-4-nitro-2-vinylbenzene (25 g, 80.64%). [M+H]⁺=198.2

### Step 6: tert-butyl 9-(5-methoxy-4-nitro-2-vinylphenyl)-3,9-diazaspiro[5.5]undecane-3-carboxylate

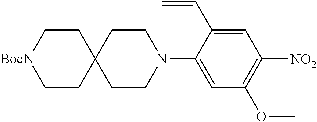 [SMILES: C=Cc1cc([N+](=O)[O-])c(OC)cc1N1CCC2(CCN(C(=O)OC(C)(C)C)CC2)CC1]

Into a 1 L round-bottom flask were placed 1-fluoro-5-methoxy-4-nitro-2-vinylbenzene (20 g, 101.52 mmol), tert-butyl 3,9-diazaspiro[5.5]undecane-3-carboxylate (25.7 g, 101.52 mmol) and DIEA (35.3 mL, 203.04 mmol) in DMSO (200 ml). The mixture was stirred for 5 h at 90° C. under nitrogen atmosphere. The mixture was extracted with EA and separated. The resulting mixture was concentrated under reduced pressure and purified by silica gel column chromatography, eluted with PE/EA (0-50%) to afford tert-butyl 9-(5-methoxy-4-nitro-2-vinylphenyl)-3,9-diazaspiro[5.5]undecane-3-carboxylate (14 g, 32.1%). [M+H]⁺=432.2.

### Step 7: tert-butyl 9-(4-amino-2-ethyl-5-methoxyphenyl)-3,9-diazaspiro[5.5]undecane-3-carboxylate

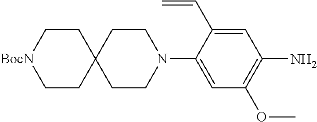 [SMILES: C=Cc1cc(N)c(OC)cc1N1CCC2(CCN(C(=O)OC(C)(C)C)CC2)CC1]

To the solution of tert-butyl 9-(5-methoxy-4-nitro-2-vinylphenyl)-3,9-diazaspiro[5.5]undecane-3-carboxylate (28 g, 64.95 mmol) in MeOH (400 mL), wet Pd/C (28 g, 10% wt) was added. To the above H₂(g) was introduced in. The resulting solution was stirred overnight at rt under H₂ atmosphere. The resulting mixture was filtered, the filter cake was washed with MeOH. The filtrate was concentrated under reduced pressure and re-crystallized from PE/EA (10:1) to afford tert-butyl 9-(4-amino-2-ethyl-5-methoxyphenyl)-3,9-diazaspiro[5.5]undecane-3-carboxylate (19.4 g, 74.61%). [M+H]⁺=404.1.

### Step 8: 3-fluoroquinolin-2(1H)-one

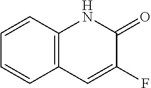 [SMILES: O=c1[nH]c2ccccc2cc1F]

To a solution of quinolin-2(1H)-one (25.0 g, 0.17 mol) in MeCN (30 mL) was added Selectfluor (64.0 g, 0.18 mol) at room temperature. The resulting mixture was stirred at 80° C. overnight. The reaction was concentrated to give the crude residue, which was purified by silica gel column chromatography (DCM:MeOH=100:0˜20:1 gradient elution) to give a mixture (10.4 g, 37%) containing the desired product. [M+H]⁺=164.2.

### Step 9: 3-fluoro-6-nitroquinolin-2(1H)-one

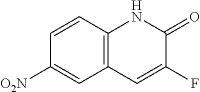 [SMILES: O=c1[nH]c2ccc([N+](=O)[O-])cc2cc1F]

To the mixture from above step (10.4 g, 63.4 mmol) in conc. H₂SO₄ (98%, 80 mL) was added conc. HNO₃ (65%, 7.4 g, 76.1 mmol) at 0° C. The resulting mixture was stirred at 0° C. for 1 hour. The reaction was poured into ice water (200 mL) and stirred for 10 mins. The mixture was filtered and the solid was washed with water (50 mL×2), dried under reduced pressure to give the crude product (7.5 g, 56.8%).

¹H NMR (500 MHz, DMSO) δ12.84 (s, 1H), 8.68 (d, J=2.5 Hz, 1H), 8.31 (dd, J=9.1, 2.5 Hz, 1H), 8.09 (d, J=10.7 Hz, 1H), 7.47 (d, J=9.1 Hz, 1H). [M+H]⁺=209.2.

### Step 10: 2-chloro-3-fluoro-6-nitroquinoline

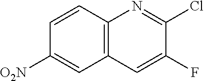 [SMILES: O=[N+]([O-])c1ccc2nc(Cl)c(F)cc2c1]

A solution of 3-fluoro-6-nitroquinolin-2(1H)-one (7.5 g, 35.9 mmol) in POCl₃ (60 mL) was heated at 100° C. overnight. The reaction was concentrated, basified with sat. aq. NaHCO₃, and then extracted with DCM (3×100 mL). The combined organic layers were dried over anhydrous Na₂SO₄, filtered and evaporated under reduced pressure to afford the crude residue, which was purified by silica gel column chromatography (PE:EA=100:0˜10:1 gradient elution) to give the desired product (3.7 g, 32%). [M+H]⁺=227.0.

### Step 11: 3-fluoro-2-methyl-6-nitroquinoline

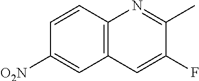

A mixture of 2-chloro-3-fluoro-6-nitroquinoline (3.7 g, 16.3 mmol), 2,4,6-trimethyl-1,3,5,2,4,6-trioxatriborinane (14.0 mL, 48.9 mmol), Pd(dppf)Cl₂ (1.2 g, 1.63 mmol) and K₃PO₄ (6.9 g, 32.6 mmol) in DME (100 mL) and H₂O (20 mL) was stirred in a round bottom flask at 80° C. overnight under nitrogen atmosphere. The mixture was evaporated under reduced pressure to afford the crude product, which was purified with silica gel column chromatography (PE:EA=100:0˜20:1 gradient elution) to give the title product (2.6 g, 77%). [M+H]⁺=207.1.

### Step 12: 3-fluoro-2-methylquinolin-6-amine

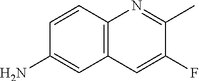

To a solution of 3-fluoro-2-methyl-6-nitroquinoline (2.6 g, 12.5 mmol) in MeOH (50 mL)/DCM (25 mL) was added Pd/C (10 wt. %, wet, 800 mg) at 25° C. under nitrogen atmosphere. The flask was evacuated and backfilled with hydrogen three times. After stirring under hydrogen atmosphere at 25° C. for 5 hours, the mixture was filtered through a pad of Celite and the solid was washed with MeOH (50 mL). The filtrate was concentrated under reduced pressure to afford the desired product (2.2 g, 99%). [M+H]⁺=177.1.

### Step 13: 3-fluoro-5-iodo-2-methylquinolin-6-amine

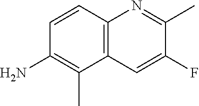

To a solution of 3-fluoro-2-methylquinolin-6-amine (2.2 g, 12.4 mmol) in AcOH (60 mL) was added ICl (16.1 mL, 16.1 mmol) at 20° C. Then the mixture was stirred at 20° C. for 1 hour. Then the mixture was adjusted to pH=8 with sat. aq. NaHCO₃. The resulting mixture was extracted with DCM (3×100 mL). The combined organic layers were washed with brine (3×80 mL), dried over Na₂SO₄, filtered and concentrated under reduced pressure to afford the desired product (3.3 g, 88%). [M+H]⁺=303.0.

### Step 14: (6-amino-3-fluoro-2-methylquinolin-5-yl)dimethylphosphine oxide

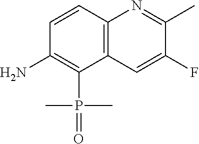 [SMILES: Cc1nc2ccc(N)c(P(C)(C)=O)c2cc1F]

To a solution of 3-fluoro-5-iodo-2-methylquinolin-6-amine (3.3 g, 10.9 mmol) and dimethylphosphine oxide (1.3 g, 16.3 mmol) in dioxane (120 mL) was added K₃PO₄ (6.9 g, 32.7 mmol) at 20° C. Pd(OAc)₂ (244 mg, 1.09 mmol) and Xantphos (1.2 g, 2.18 mmol) were then added to the mixture at the same temperature. The flask was evacuated and backfilled with nitrogen three times, before the mixture was stirred at 100° C. overnight. The mixture was filtered and concentrated under reduced pressure. The residue was purified by silica gel column chromatography (DCM:MeOH=100:0˜10:1 gradient elution) to give the desired product (2.5 g, 91%). [M+H]⁺=253.1.

### Step 15: (6-((5-bromo-2-chloropyrimidin-4-yl)amino)-3-fluoro-2-methylquinolin-5-yl)dimethylphosphine oxide

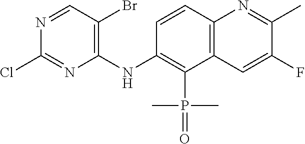 [SMILES: Cc1nc2ccc(Nc3nc(Cl)ncc3Br)c(P(C)(C)=O)c2cc1F]

To a solution of (6-amino-3-fluoro-2-methylquinolin-5-yl)dimethylphosphine oxide (2.5 g, 9.9 mmol) and 5-bromo-2,4-dichloropyrimidine (6.7 g, 29.7 mmol) in n-BuOH (100 mL) was added DIEA (3.8 g, 29.7 mmol) at room temperature. The resulting mixture was stirred at 120° C. overnight. The reaction was concentrated to afford the crude residue, which was purified by silica gel column chromatography (DCM:MeOH=100:0˜15:1 gradient elution) to give the desired product (2.9 g, 66%). [M+H]⁺=443.1.

### Step 16: (6-((5-bromo-2-((5-ethyl-2-methoxy-4-(3,9-diazaspiro[5.5]undecan-3-yl)phenyl)amino)pyrimidin-4-yl)amino)-3-fluoro-2-methylquinolin-5-yl)dimethylphosphine oxide

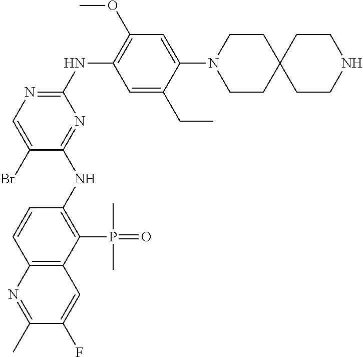 [SMILES: CCc1cc(Nc2ncc(Br)c(Nc3ccc4nc(C)c(F)cc4c3P(C)(C)=O)n2)c(OC)cc1N1CCC2(CCNCC2)CC1]

To the solution of (6-((5-bromo-2-chloropyrimidin-4-yl)amino)-3-fluoro-2-methylquinolin-5-yl)dimethylphosphine oxide (4.9 g, 11 mmol), tert-butyl 9-(4-amino-2-ethyl-5-methoxyphenyl)-3,9-diazaspiro[5.5]undecane-3-carboxylate (4.8 g, 12 mmol) and TsOH (4.8 g, 28 mmol) in 70 mL n-BuOH and 10 mL CF₃CH₂OH. The mixture was stirred at 100° C. for 16 hours. The mixture was concentrated in vacuum. The solid was diluted in DCM and water and separated. The aqueous was adjusted pH=6-7 with 1N NaOH aqueous and filtered. The solid was purified by combiflash (MeOH with 1% NH₃·H₂O:DCM=0-20%) to afford the titled compound (5.7 g, 73% yield); [M+H]⁺=710.9.

### Step 9: (R)-3-(4-(4-(9-(4-((5-bromo-4-((5-(dimethylphosphoryl)-3-fluoro-2-methylquinolin-6-yl)amino)pyrimidin-2-yl)amino)-2-ethyl-5-methoxyphenyl)-3,9-diazaspiro[5.5]undecan-3-yl)piperidin-1-yl)-2,6-difluorophenyl)piperidine-2,6-dione

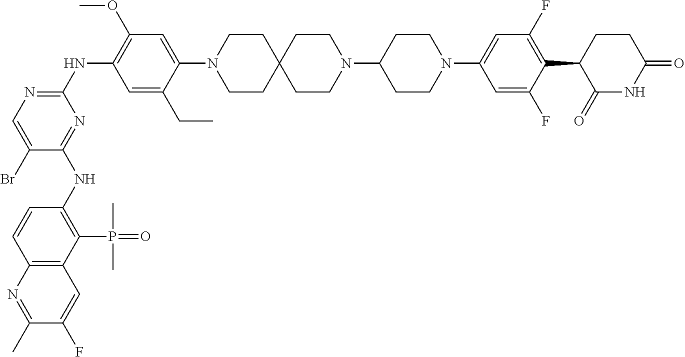 [SMILES: CCc1cc(Nc2ncc(Br)c(Nc3ccc4nc(C)c(F)cc4c3P(C)(C)=O)n2)c(OC)cc1N1CCC2(CC1)CCN(C1CCN(c3cc(F)c([C@H]4CCC(=O)NC4=O)c(F)c3)CC1)CC2]

To the solution of (6-((5-bromo-2-((5-ethyl-2-methoxy-4-(3,9-diazaspiro[5.5]undecan-3-yl)phenyl)amino)pyrimidin-4-yl)amino)-3-fluoro-2-methylquinolin-5-yl)dimethylphosphine oxide (1.42 g, 2 mmol) and (R)-3-(2,6-difluoro-4-(4-oxopiperidin-1-yl)phenyl)piperidine-2,6-dione (805 mg, 2.5 mmol) in 30 mL DCE and 5 mL DMSO, 3 mL Ti(OiPr)₄ was added. The mixture was stirred at 40° C. for 2 hours. Then, NaBH(OAc)₃ (1.27 g, 6 mmol) was added. The mixture was stirred at 40° C. for 1 hour. The mixture was quenched with NaHCO₃ aqueous and filtered. The organic layer was separated and concentrated in vacuum. The crude product was purified by combiflash (MeOH:DCM=0-8%) to afford the title compound (1.2 g, 59% yield).

¹H NMR (500 MHz, DMSO) δ 11.34 (s, 1H), 10.87 (s, 1H), 8.60 (d, J=11.6 Hz, 1H), 8.27-8.15 (m, 2H), 8.01-7.89 (m, 2H), 7.30 (s, 1H), 6.78 (s, 1H), 6.63 (d, J=13.0 Hz, 2H), 4.04 (dd, J=11.8, 4.8 Hz, 1H), 3.86-3.72 (m, 5H), 3.31-3.24 (m, 2H), 2.87-2.67 (m, 8H), 2.67-2.57 (m, 4H), 2.39-2.49 (m, 2H), 2.26-2.15 (m, 2H), 2.12-2.03 (m, 1H), 1.99-1.88 (m, 7H), 1.87-1.73 (m, 2H), 1.65-1.39 (m, 10H), 0.72 (s, 3H); [M+H]⁺=1016.7.

### Example 2: (R)-3-(4-(4-(9-(4-((5-chloro-4-((5-(dimethylphosphoryl)-3-fluoro-2-methylquinolin-6-yl)amino)pyrimidin-2-yl)amino)-2-ethyl-5-methoxyphenyl)-3,9-diazaspiro[5.5]undecan-3-yl)piperidin-1-yl)-2,6-difluorophenyl)piperidine-2,6-dione

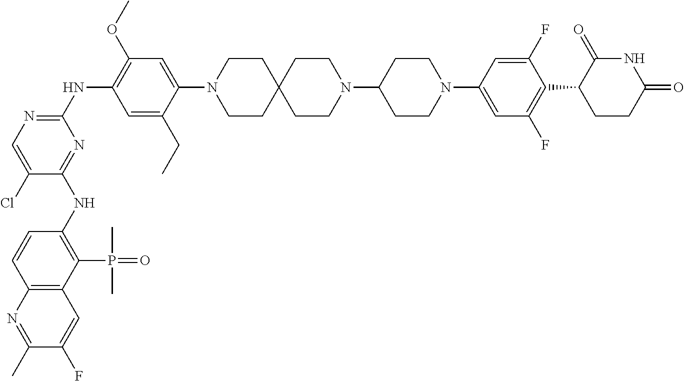 [SMILES: CCc1cc(Nc2ncc(Cl)c(Nc3ccc4nc(C)c(F)cc4c3P(C)(C)=O)n2)c(OC)cc1N1CCC2(CC1)CCN(C1CCN(c3cc(F)c([C@H]4CCC(=O)NC4=O)c(F)c3)CC1)CC2]

The title compound was prepared in a procedure similar to that in Example 1. H NMR (500 MHz, DMSO) δ 11.67 (s, 1H), 10.87 (s, 1H), 8.54 (d, J=9.5 Hz, 1H), 8.36 (s, 1H), 8.15 (s, 1H), 8.00 (s, 1H), 7.94 (d, J=9.4 Hz, 1H), 7.33 (s, 1H), 6.80 (s, 1H), 6.65 (s, 2H), 4.05 (dd, J=12.3, 4.5 Hz, 1H), 3.85-3.73 (m, 5H), 2.83-2.67 (m, 8H), 2.64 (s, 3H), 2.51 (s, 2H), 2.47-2.38 (m, 1H), 2.25 (d, J=6.3 Hz, 2H), 2.13-2.04 (m, 2H), 2.00-1.93 (m, 8H), 1.82 (s, 2H), 1.70-1.43 (m, 10H), 0.77 (s, 3H). [M+H]⁺=972.7.

### Example 3: (R)-3-(4-(4-(9-(4-((5-bromo-4-((5-(dimethylphosphoryl)-2-ethylquinazolin-6-yl)amino)pyrimidin-2-yl)amino)-2-ethyl-5-methoxyphenyl)-3,9-diazaspiro[5.5]undecan-3-yl)piperidin-1-yl)-2,6-difluorophenyl)piperidine-2,6-dione

### Step 1: 2-ethyl-6-nitroquinazoline

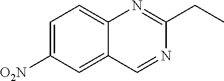

To a solution of 2-fluoro-5-nitrobenzaldehyde (10.0 g, 59.17 mmol) in MeCN (150 mL) was added propionimidamide hydrochloride (9.59 g, 88.75 mmol) and K₂CO₃ (20.4 g, 147.93 mmol) at room temperature. The resulting mixture was stirred at 80° C. overnight. The reaction was concentrated to give the crude residue, which was purified by silica gel column chromatography (DCM:MeOH=100:0˜98:2 gradient elution) to give a mixture (3.6 g, 30%) containing the desired product. [M+H]⁺=204.2.

### Step 2: 2-ethylquinazolin-6-amine

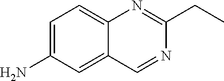

To a solution of 2-ethyl-6-nitroquinazoline (3.6 g, 17.73 mmol) in THF (100 mL)/H₂O (20 mL) was added Fe (4.96 g, 88.67 mmol) and NH₄Cl (4.7 g, 88.67 mmol) at 25° C. Then the mixture was stirred at 25° C. overnight. The mixture was filtered through a pad of Celite and washed with EA (150 mL) and H₂O (60 mL). The filtrate was separated and the organic layer was concentrated to give the crude residue, which was purified by silica gel column chromatography (DCM:MeOH=100:0˜20:1 gradient elution) to give a mixture (1.8 g, 58%) containing the desired product. [M+H]⁺=174.2.

### Step 3: 2-ethyl-5-iodoquinazolin-6-amine

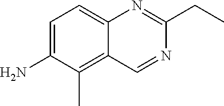

To a solution of 2-ethylquinazolin-6-amine (1.8 g, 10.4 mmol) in AcOH (30 mL) was added ICl (15.6 mL, 15.6 mmol) at 20° C. Then the mixture was stirred at 20° C. for 3 hours. Then the mixture was adjusted to pH=8 with sat. aq. NaHCO₃ and extracted with DCM (2×100 mL). The organic phase was washed with brine (80 mL), dried over Na₂SO₄, filtered and concentrated in vacuum to afford the desired product (2.6 g, 84%). [M+H]⁺=300.1.

### Step 4: (6-amino-2-ethylquinazolin-5-yl)dimethylphosphine oxide

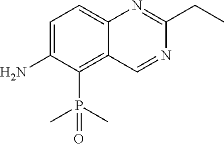 [SMILES: CCc1ncc2c(P(C)(C)=O)c(N)ccc2n1]

To a solution of 2-ethyl-5-iodoquinazolin-6-amine (2.6 g, 8.69 mmol) and dimethylphosphine oxide (1.36 g, 17.39 mmol) in dioxane (100 mL) was added K₃PO₄ (4.6 g, 21.73 mmol) at 20° C. Pd(OAc)₂ (390 mg, 1.74 mmol) and Xantphos (1.0 g, 1.74 mmol) were added to the mixture at 20° C. The suspension was degassed under vacuum and purged with N₂ three times. Then the mixture was stirred at 100° C. overnight.

The mixture was filtered and concentrated in vacuum. The residue was purified by silica gel column chromatography (DCM:MeOH=100:0˜10:1 gradient elution) to give the desired product (2.1 g, 97%). [M+H]⁺=250.1.

### Step 5: (6-((5-bromo-2-chloropyrimidin-4-yl)amino)-2-ethylquinazolin-5-yl)dimethylphosphine oxide

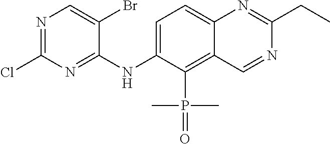 [SMILES: CCc1ncc2c(P(C)(C)=O)c(Nc3nc(Cl)ncc3Br)ccc2n1]

To a solution of (6-amino-2-ethylquinazolin-5-yl)dimethylphosphine oxide (2.1 g, 8.4 mmol) and 5-bromo-2,4-dichloropyrimidine (5.7 g, 25.2 mmol) in n-BuOH (90 mL) was added DIEA (3.3 g, 25.2 mmol) at room temperature. The resulting mixture was stirred at 120° C. overnight. The reaction was concentrated to afford the crude residue, which was purified by silica gel column chromatography (DCM:MeOH=100:0˜15:1 gradient elution) to give the desired product (2.3 g, 62%).

¹H NMR (500 MHz, DMSO) δ 12.44 (s, 1H), 9.83 (s, 1H), 8.59 (s, 1H), 8.56 (dd, J=9.3, 3.8 Hz, 1H), 8.12 (d, J=9.3 Hz, 1H), 3.07 (q, J=7.6 Hz, 2H), 2.12-2.08 (m, 6H), 1.38 (t, J=7.6 Hz, 3H). [M+H]⁺=440.1.

### Step 6: (6-((5-bromo-2-((5-ethyl-2-methoxy-4-(3,9-diazaspiro[5.5]undecan-3-yl)phenyl)amino)pyrimidin-4-yl)amino)-2-ethylquinazolin-5-yl)dimethylphosphine oxide

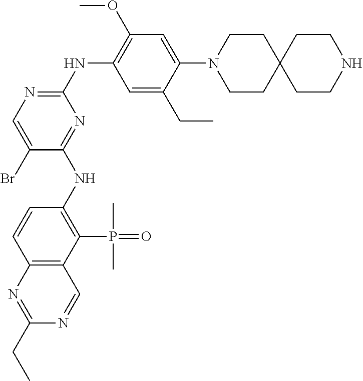 [SMILES: CCc1ncc2c(P(C)(C)=O)c(Nc3nc(Nc4cc(CC)c(N5CCC6(CCNCC6)CC5)cc4OC)ncc3Br)ccc2n1]

The title compound (410 mg, 62%) was prepared from (6-((5-bromo-2-chloropyrimidin-4-yl)amino)-2-ethylquinazolin-5-yl)dimethylphosphine oxide and tert-butyl 9-(4-amino-2-ethyl-5-methoxyphenyl)-3,9-diazaspiro[5.5]undecane-3-carboxylate according to the same manner as shown in step 8 of example 1.

### Step 7: (R)-3-(4-(4-(9-(4-((5-bromo-4-((5-(dimethylphosphoryl)-2-ethylquinazolin-6-yl)amino)pyrimidin-2-yl)amino)-2-ethyl-5-methoxyphenyl)-3,9-diazaspiro[5.5]undecan-3-yl)piperidin-1-yl)-2,6-difluorophenyl)piperidine-2,6-dione

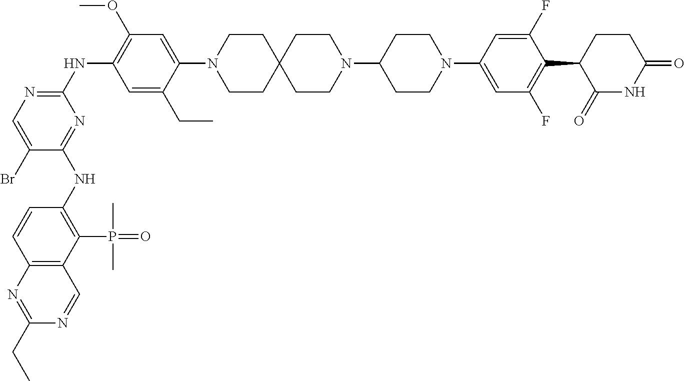 [SMILES: CCc1ncc2c(P(C)(C)=O)c(Nc3nc(Nc4cc(CC)c(N5CCC6(CC5)CCN(C5CCN(c7cc(F)c([C@H]8CCC(=O)NC8=O)c(F)c7)CC5)CC6)cc4OC)ncc3Br)ccc2n1]

To the solution of (6-((5-bromo-2-((5-ethyl-2-methoxy-4-(3,9-diazaspiro[5.5]undecan-3-yl)phenyl)amino)pyrimidin-4-yl)amino)-2-ethylquinazolin-5-yl)dimethylphosphine oxide (70 mg, 0.1 mmol) and (R)-3-(2,6-difluoro-4-(4-oxopiperidin-1-yl)phenyl)piperidine-2,6-dione (40 mg, 0.13 mmol) in 3 mL DCE, 1 drop of Ti(OiPr)₄ was added. The mixture was stirred at 40° C. for 2 hours. Then, NaBH(OAc)₃ (40 mg, 0.2 mmol) was added. The mixture was stirred at 40° C. for 1 hour. The mixture was quenched with NaHCO₃ aqueous and filtered. The organic layer was separated and concentrated in vacuum. The crude product was purified by prep-TLC (DCM:MeOH=11:1) to afford the title compound (8 mg, 8% yield).

¹H NMR (500 MHz, DMSO) δ 11.57 (s, 1H), 10.88 (s, 1H), 9.89 (s, 1H), 8.48 (s, 1H), 8.24 (s, 1H), 8.03 (s, 1H), 7.86 (d, J=9.1 Hz, 1H), 7.30 (s, 1H), 6.82 (s, 1H), 6.72 (d, J=12.9 Hz, 2H), 4.10-4.04 (m, 1H), 4.01-3.95 (m, 2H), 3.77 (s, 3H), 3.48-3.33 (m, 4H), 3.17-3.01 (m, 4H), 2.85-2.71 (m, 7H), 2.30-2.21 (m, 2H), 2.17-2.07 (m, 4H), 2.06-2.01 (m, 7H), 1.99-1.91 (m, 3H), 1.73-1.62 (m, 4H), 1.54-1.47 (m, 2H), 1.38 (t, J=7.5 Hz, 3H), 0.73 (s, 3H); [M+H]⁺=1013.7.

### Example 4: (R)-3-(4-(9-(1-(4-((5-bromo-4-((5-(dimethylphosphoryl)-2-ethylquinazolin-6-yl)amino)pyrimidin-2-yl)amino)-2-ethyl-5-methoxyphenyl)piperidin-4-yl)-3,9-diazaspiro[5.5]undecan-3-yl)-2,6-difluorophenyl)piperidine-2,6-dione

### Step 1: tert-butyl 9-(4-(2,6-bis(benzyloxy)pyridin-3-yl)-3,5-difluorophenyl)-3,9-diazaspiro[5.5]undecane-3-carboxylate

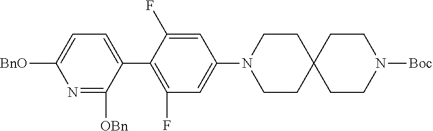 [SMILES: CC(C)(C)OC(=O)N1CCC2(CC1)CCN(c1cc(F)c(-c3ccc(OCc4ccccc4)nc3OCc3ccccc3)c(F)c1)CC2]

To the solution of 2,6-bis(benzyloxy)-3-(4-bromo-2,6-difluorophenyl)pyridine (2 g, 4.15 mmol), tert-butyl 3,9-diazaspiro[5.5]undecane-3-carboxylate (1.27 g, 5 mmol) and Cs₂CO₃ (2.6 g, 8 mmol) in 20 mL dioxane, Pd₂(dba)₃ (183 mg, 0.2 mmol) and Xantphos (231 mg, 0.4 mmol) was added under N₂ protected. The mixture was stirred at 80° C. for 16 hours at N₂ atmosphere. The mixture was filtered and concentrated in vacuum. The mixture was purified by combiflash (EA:PE=0-20%) to afford the title compound (1.6 g, 59% yield); [M+H]⁺=655.9.

### Step 2: tert-butyl 9-(4-(2,6-dioxopiperidin-3-yl)-3,5-difluorophenyl)-3,9-diazaspiro[5.5]undecane-3-carboxylate

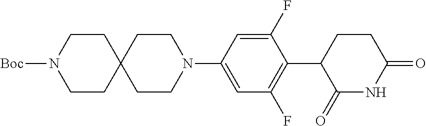 [SMILES: CC(C)(C)OC(=O)N1CCC2(CC1)CCN(c1cc(F)c(C3CCC(=O)NC3=O)c(F)c1)CC2]

To the solution of tert-butyl 9-(4-(2,6-bis(benzyloxy)pyridin-3-yl)-3,5-difluorophenyl)-3,9-diazaspiro[5.5]undecane-3-carboxylate (1.6 g, 2.44 mmol) in 10 mL DCM and 60 mL iPrOH, Pd/C (1.6 g, 10 wt. %, wet) was added. The mixture was stirred at 45° C. for 16 hours at H₂ atmosphere. The mixture was filtered directly and concentrated to afford the title compound (860 mg, 74% yield); [M+H]⁺=477.9.

### Step 3: 3-(2,6-difluoro-4-(3,9-diazaspiro[5.5]undecan-3-yl)phenyl)piperidine-2,6-dione hydrochloride

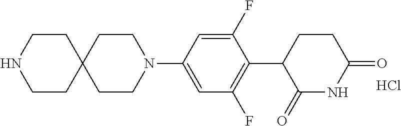 [SMILES: O=C1CCC(c2c(F)cc(N3CCC4(CCNCC4)CC3)cc2F)C(=O)N1 ; Cl]

To the solution of tert-butyl 9-(4-(2,6-dioxopiperidin-3-yl)-3,5-difluorophenyl)-3,9-diazaspiro[5.5]undecane-3-carboxylate (60 mg, 0.12 mmol) in 0.5 mL DCM, 5 mL 4N HCl/dioxane was added. The mixture was stirred at room temperature for 1 hour. The mixture was concentrated in vacuum to afford the title compound (40 mg, 88% yield); [M+H]⁺=377.9.

### Step 4: (R)-3-(4-(9-(1-(4-((5-bromo-4-((5-(dimethylphosphoryl)-2-ethylquinazolin-6-yl)amino)pyrimidin-2-yl)amino)-2-ethyl-5-methoxyphenyl)piperidin-4-yl)-3,9-diazaspiro[5.5]undecan-3-yl)-2,6-difluorophenyl)piperidine-2,6-dione

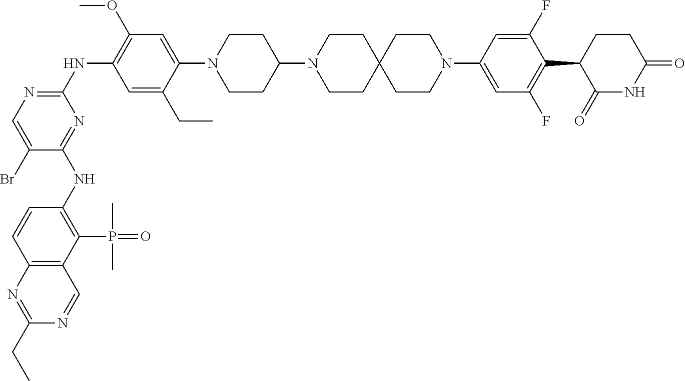 [SMILES: CCc1ncc2c(P(C)(C)=O)c(Nc3nc(Nc4cc(CC)c(N5CCC(N6CCC7(CCN(c8cc(F)c([C@H]9CCC(=O)NC9=O)c(F)c8)CC7)CC6)CC5)cc4OC)ncc3Br)ccc2n1]

To the solution of 1-(4-((5-bromo-4-((5-(dimethylphosphoryl)-2-ethylquinazolin-6-yl)amino)pyrimidin-2-yl)amino)-2-ethyl-5-methoxyphenyl)piperidin-4-one (60 mg, 0.1 mmol), 3-(2,6-difluoro-4-(3,9-diazaspiro[5.5]undecan-3-yl)phenyl)piperidine-2,6-dione hydrochloride (40 mg, 0.1 mmol) and DIEA (129 mg, 1 mmol) in 1.5 mL DMSO. The mixture was stirred at 70° C. for 16 hours. Then, NaBH(OAc)₃ (60 mg, 0.3 mmol) was added. The mixture was stirred at room temperature for 2 hours. The mixture was diluted with DCM and washed with saturated brine. The organic layer was concentrated in vacuum and purified by prep-TLC (DCM:MeOH=11:1). The crude product was purified by SFC (IF (2 cm×25 cm, 5 um), MtBE (0.1% DEA):(MeOH:DCM=1:1)=50:50, 100 bar, 20 ml/min) and the title compound corresponded to peak A @ 1.402 min/254 nm (28 mg, 27% yield).

¹H NMR (500 MHz, DMSO) δ 11.62 (s, 1H), 10.86 (s, 1H), 9.86 (s, 1H), 8.46 (s, 1H), 8.23 (s, 1H), 8.05 (s, 1H), 7.85 (d, J=8.9 Hz, 1H), 7.28 (s, 1H), 6.74 (s, 1H), 6.61 (d, J=13.0 Hz, 2H), 4.04 (dd, J=12.0, 4.1 Hz, 1H), 3.75 (s, 3H), 3.31-3.26 (m, 1H), 3.20 (s, 4H), 3.09-3.01 (m, 2H), 2.98-2.90 (m, 2H), 2.84-2.73 (m, 1H), 2.71-2.62 (m, 2H), 2.44-2.35 (m, 4H), 2.30-2.23 (m, 2H), 2.14-1.90 (m, 9H), 1.88-1.78 (m, 2H), 1.66-1.56 (m, 2H), 1.55-1.43 (m, 8H), 1.42-1.35 (m, 3H), 0.76 (s, 3H); [M+H]⁺=1013.5.

### Example 5: (R)-3-(4-(3-(9-(4-((5-bromo-4-((5-(dimethylphosphoryl)-3-fluoro-2-methylquinolin-6-yl)amino)pyrimidin-2-yl)amino)-2-ethyl-5-methoxyphenyl)-3,9-diazaspiro[5.5]undecan-3-yl)cyclobutyl)-2,6-difluorophenyl)piperidine-2,6-dione

### Step 1: 2,6-bis(benzyloxy)-3-(2,6-difluoro-4-(5,8-dioxaspiro[3.4]octan-2-yl)phenyl)pyridine

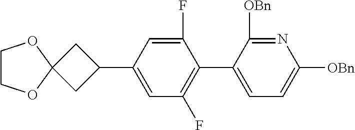 [SMILES: Fc1cc(C2CC3(C2)OCCO3)cc(F)c1-c1ccc(OCc2ccccc2)nc1OCc1ccccc1]

To a solution of 2,6-bis(benzyloxy)-3-(4-bromo-2,6-difluorophenyl)pyridine (1 g, 1.94 mmol), 2-bromo-5,8-dioxaspiro[3.4]octane (598.8 mg, 2.91 mmol) in DMA (10 mL), were added NiI₂ (121.7 mg, 0.39 mmol), picolinimidamide (61.2 mg, 0.39 mmol), NaI (144.53 mg, 0.97 mmol), Mn (320.1 mg, 5.82 mmol). After being protected by nitrogen gas, a solution of TFA (29 mL, 0.39 mmol) in DMA (1 mL) was added. The mixture was stirred at 100° C. for 3 h under nitrogen atmosphere. After cooled to room temperature, the reaction was quenched with water and extracted with ethyl acetate. The combined organic layers were dried over anhydrous Na₂SO₄. After filtration, the filtrate was concentrated under reduced pressure. The residue was purified by C18 column chromatography (ACN/0.05% TFA in H₂O, 75% to 80% in 40 min) to afford 2,6-bis(benzyloxy)-3-(2,6-difluoro-4-(5,8-dioxaspiro[3.4]octan-2-yl)phenyl)pyridine (400 mg, 37.3%). [M+H]⁺=516.2.

### Step 2: 3-(2,6-difluoro-4-(5,8-dioxaspiro[3.4]octan-2-yl)phenyl)piperidine-2,6-dione

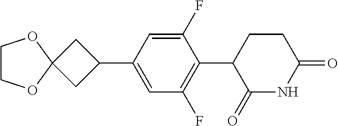 [SMILES: O=C1CCC(c2c(F)cc(C3CC4(C3)OCCO4)cc2F)C(=O)N1]

To a solution of 2,6-bis(benzyloxy)-3-(2,6-difluoro-4-(5,8-dioxaspiro[3.4]octan-2-yl)phenyl)pyridine (400 mg, 0.78 mmol) in THF (10 mL), were added Pd/C (1 g, 10% wt, wet). The mixture was stirred overnight at 50° C. under hydrogen atmosphere. After cooled to room temperature. The resulting mixture was filtered, the filter cake was washed with MeOH. The filtrate was concentrated under reduced pressure and re-crystallized from PE/EA (10:1) to afford 3-(2,6-difluoro-4-(5,8-dioxaspiro[3.4]octan-2-yl)phenyl)piperidine-2,6-dione (99.1 mg, 37.9%). [M+H]⁺=338.3

### Step 3: 3-(2,6-difluoro-4-(3-oxocyclobutyl)phenyl)piperidine-2,6-dione

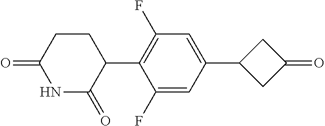 [SMILES: O=C1CC(c2cc(F)c(C3CCC(=O)NC3=O)c(F)c2)C1]

To a stirred solution of 3-(2,6-difluoro-4-(5,8-dioxaspiro [3.4] octan-2-yl) phenyl) piperidine-2,6-dione (200 mg, 0.592 mmol) in dioxane:H₂O=2:1 (2 mL) was added 1M HCl (in dioxane) (2 mL). The reaction mixture was stirred at rt for 2 h and concentrated in vacuum to afford the product (165 mg, 94.8%) [M+H]⁺=294.2.

### Step 4: (R)-3-(4-(3-(9-(4-((5-bromo-4-((5-(dimethylphosphoryl)-3-fluoro-2-methylquinolin-6-yl)amino)pyrimidin-2-yl)amino)-2-ethyl-5-methoxyphenyl)-3,9-diazaspiro[5.5]undecan-3-yl)cyclobutyl)-2,6-difluorophenyl)piperidine-2,6-dione

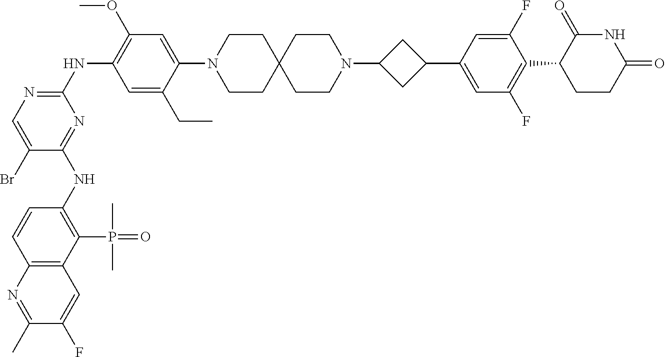 [SMILES: CCc1cc(Nc2ncc(Br)c(Nc3ccc4nc(C)c(F)cc4c3P(C)(C)=O)n2)c(OC)cc1N1CCC2(CC1)CCN(C1CC(c3cc(F)c([C@H]4CCC(=O)NC4=O)c(F)c3)C1)CC2]

To a solution of (6-((5-bromo-2-((5-ethyl-2-methoxy-4-(3,9-diazaspiro[5.5]undecan-3-yl)phenyl)amino)pyrimidin-4-yl)amino)-3-fluoro-2-methylquinolin-5-yl)dimethylphosphine oxide (100 mg, 0.14 mmol) and 3-(2,6-difluoro-4-(3-oxocyclobutyl)phenyl)piperidine-2,6-dione (49.24 mg, 0.168 mmol) in DCE (3 mL) was added NaBH(OAc)₃ (89.01 mg, 0.42 mmol) at 20° C. The resulting mixture was stirred at room temperature for 1 hour. The reaction was quenched with water (10 mL) and the layers were separated. The aqueous layer was extracted with DCM (3×10 mL). The combined organic layers were dried over anhydrous Na₂SO₄, filtered and evaporated in vacuum to afford the crude residue, which was purified by silica gel column chromatography (DCM:MeOH=100:0˜10:1 gradient elution) to give an impure product, which was separated by chiral-HPLC (CHIRALPAK IF-3 4.6*50 mm, 3 μm), MtBE (0.1% DEA):(MeOH:DCM=1:1)=80:20, 1 ml/min) and the title compound corresponded to peak A @ 2.951 min/254 nm) (8 mg, 8.1%). ¹H NMR (500 MHz, DMSO) δ 11.35 (s, 1H), 10.95 (s, 1H), 8.61 (s, 1H), 8.22 (s, 2H), 7.97 (s, 2H), 7.30 (s, 1H), 6.98 (s, 2H), 6.78 (s, 1H), 4.21 (s, 1H), 3.72-3.76 (m, 3H), 3.15-3.17 (m, 1H), 2.72 (s, 6H), 2.29-2.31 (m, 4H), 2.20-2.22 (m, 2H), 1.96-1.98 (m, 8H), 1.86 (s, 2H), 1.54 (s, 8H), 1.21-1.24 (m, 4H), 0.86 (s, 2H), 0.73 (s, 3H). [M+H]⁺=987.5.

### Example 6: (R)-3-(4-(2-(9-(4-((5-bromo-4-((5-(dimethylphosphoryl)-3-fluoro-2-methylquinolin-6-yl)amino)pyrimidin-2-yl)amino)-2-ethyl-5-methoxyphenyl)-3,9-diazaspiro[5.5]undecan-3-yl)ethyl)-2,6-difluorophenyl)piperidine-2,6-dione

### Step 1: ethyl 4-(4-bromo-2,6-difluorophenyl)-4-cyanobutanoate

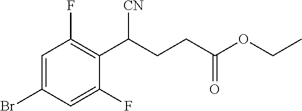 [SMILES: CCOC(=O)CCC(C#N)c1c(F)cc(Br)cc1F]

To a solution of 2-(4-bromo-2,6-difluorophenyl)acetonitrile (10 g, 43.1 mmol) in THF (150 mL) was added LDA (2M, 24 mL, 48 mmol) dropwise in 20 min at −65° C., the reaction solution was stirred for 1 hour at this temperature, then to this was added ethyl 3-bromopropanoate (9.4 g, 51.7 mmol) in THF (30 mL) dropwise in 10 min. The resulting solution was stirred for 30 min at −65° C., then the temperature was allowed to rise to room temperature naturally. The reaction was quenched by the addition of sat. aq. NH₄Cl (50 mL), and the layers were separated. The aqueous layer was extracted with EtOAc (100 mL×3), the organic layers were combined and washed with brine, dried over anhydrous Na₂SO₄. After filtration, the filtrate was concentrated under reduced pressure to afford the product (13.8 g, 96.5%). [M+H]⁺=332.0.

### Step 2: 4-(4-bromo-2,6-difluorophenyl)-4-cyanobutanoic acid

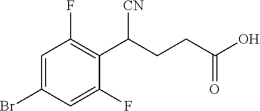 [SMILES: N#CC(CCC(=O)O)c1c(F)cc(Br)cc1F]

To a solution of ethyl 4-(4-bromo-2,6-difluorophenyl)-4-cyanobutanoate (13.5 g, 40.7 mmol) in THF/H₂O (90 mL/30 mL) was added LiOH (2.9 g, 0.122 mol). The reaction mixture was stirred for 12 h at room temperature. The resulting mixture was diluted with water, and the layers were separated. The pH value of aqueous layer was adjusted to 4-5 with 1 M HCl, and then extracted with EtOAc (50 mL×3). The combined organic layers were washed with brine (50 mL), dried over anhydrous Na₂SO₄. After filtration, the filtrate was concentrated under reduced pressure to afford the product (10.2 g, 82.5%). [M+H]⁺=304.2.

### Step 3: 3-(4-bromo-2,6-difluorophenyl)piperidine-2,6-dione

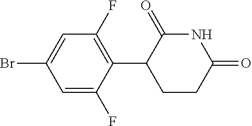 [SMILES: O=C1CCC(c2c(F)cc(Br)cc2F)C(=O)N1]

To a stirred solution of 4-(4-bromo-2,6-difluorophenyl)-4-cyanobutanoic acid (10.2 g, 33.5 mmol) in toluene (100 mL) was added conc. H₂SO₄ (2 mL, 36.9 mmol). The resulting solution was stirred at 100° C. for 3 h. The reaction mixture was concentrated under vacuum, then the mixture was poured into water. The pH value was adjusted to 7-8 with sat. aq. NaHCO₃, and then the mixture was extracted with EtOAc (50 mL×3). The combined organic layers were washed with water (50 mL) and brine (50 mL), dried over anhydrous Na₂SO₄, filtered and concentrated under reduced pressure to afford the product (8.2 g, 80.4%). [M+H]⁺=304.3.

### Step 4: (R)-3-(4-(2-ethoxyvinyl)-2,6-difluorophenyl)piperidine-2,6-dione

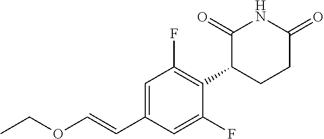 [SMILES: CCO/C=C/c1cc(F)c([C@H]2CCC(=O)NC2=O)c(F)c1]

To a stirred solution of 3-(4-bromo-2,6-difluorophenyl)piperidine-2,6-dione (8.2 g, 27.0 mmol) and (E)-2-(2-ethoxyvinyl)-4,4,5,5-tetramethyl-1,3,2-dioxaborolane (6.4 g, 32.4 mmol) in DMF/H₂O (100 mL/20 mL) were added Pd(dtbpf)Cl₂ (883 mg, 1.35 mmol) and CsF (8.2 g, 54.0 mmol). The resulting mixture was stirred for 2 h at 80° C. under nitrogen atmosphere. The reaction solution was diluted with water (400 mL), and extracted with EtOAc (100 mL×2). The combined organic layers were washed with water (100 mL) and brine (100 mL), dried over anhydrous Na₂SO₄, filtered and concentrated under reduced pressure. The residue was purified by SFC ((IH(3*25 cm, 5 um), 13% EtOH/87% CO₂, 100 bar, 100 ml/min) to afford (S,E)-3-(4-(2-ethoxyvinyl)-2,6-difluorophenyl)piperidine-2,6-dione corresponded to peak B @ 2.049 min/254 nm (3.1 g, 39.0%) and (R)-3-(4-(2-ethoxyvinyl)-2,6-difluorophenyl)piperidine-2,6-dione corresponded to peak A @1.679 min/254 nm (2.9 g, 36.5%). (S)-3-(4-(2-ethoxyvinyl)-2,6-difluorophenyl)piperidine-2,6-dione and (R,E)-3-(4-(2-ethoxyvinyl)-2,6-difluorophenyl)piperidine-2,6-dione had the same ¹H NMR and LCMS data. ¹H NMR (500 MHz, DMSO) δ 10.92 (s, 1H), 7.41 (d, J=12.9 Hz, 1H), 7.06 (d, J=10.7 Hz, 2H), 5.82 (d, J=12.9 Hz, 1H), 4.17-4.13 (m, 1H), 3.92-3.88 (m, 2H), 3.45-3.39 (m, 1H), 2.82-2.76 (m, 1H), 2.12-2.07 (m, 1H), 2.00-1.96 (m, 1H), 1.26 (t, J=7.0 Hz, 3H). [M+H]⁺=295.9.

### Step 5: (R)-2-(4-(2,6-dioxopiperidin-3-yl)-3,5-difluorophenyl)acetaldehyde

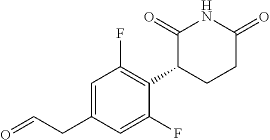 [SMILES: O=CCc1cc(F)c([C@H]2CCC(=O)NC2=O)c(F)c1]

(R)-3-(4-(2-ethoxyvinyl)-2,6-difluorophenyl)piperidine-2,6-dione (3.1 g, 10.4 mmol) was dissolved in FA (50 mL). The resulting solution was stirred for 2 h at room temperature. The reaction solution was evaporated to dryness to afford the product (2.6 g, 91.8%). [M+H]⁺=268.1.

### Step 6: (R)-3-(4-(2-(9-(4-((5-bromo-4-((5-(dimethylphosphoryl)-3-fluoro-2-methylquinolin-6-yl)amino)pyrimidin-2-yl)amino)-2-ethyl-5-methoxyphenyl)-3,9-diazaspiro[5.5]undecan-3-yl)ethyl)-2,6-difluorophenyl)piperidine-2,6-dione

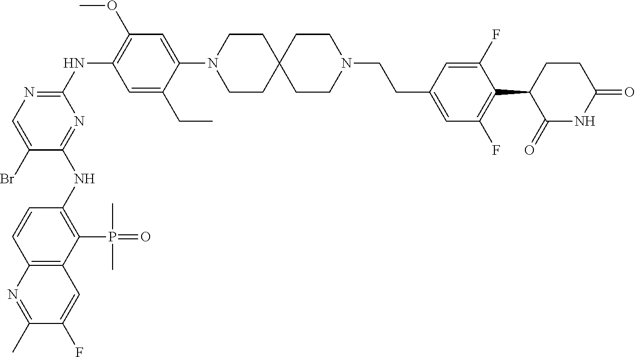 [SMILES: CCc1cc(Nc2ncc(Br)c(Nc3ccc4nc(C)c(F)cc4c3P(C)(C)=O)n2)c(OC)cc1N1CCC2(CCN(CCc3cc(F)c([C@H]4CCC(=O)NC4=O)c(F)c3)CC2)CC1]

To the solution of (6-((5-bromo-2-((5-ethyl-2-methoxy-4-(3,9-diazaspiro[5.5]undecan-3-yl)phenyl)amino)pyrimidin-4-yl)amino)-3-fluoro-2-methylquinolin-5-yl)dimethylphosphine oxide (1.4 g, 2 mmol) and (R)-2-(4-(2,6-dioxopiperidin-3-yl)-3,5-difluorophenyl)acetaldehyde (667 mg, 2.5 mmol) in 20 mL DCM, the mixture was stirred at room temperature for 30 minutes. Then, NaBH(OAc)₃ (1.27 g, 6 mmol) was added. The mixture was stirred at room temperature for 30 minutes. The mixture was diluted with DCM and washed with water. The organic layer was separated and concentrated in vacuum. The crude product was purified by combiflash (MeOH:DCM=0-8%) to afford the title compound (1.3 g, 67% yield). ¹H NMR (500 MHz, DMSO) δ 11.33 (s, 1H), 10.95 (s, 1H), 8.61 (d, J=12.4 Hz, 1H), 8.24-8.16 (m, 2H), 7.99-7.92 (m, 2H), 7.30 (s, 1H), 7.03 (d, J=9.7 Hz, 2H), 6.79 (s, 1H), 4.24-4.18 (m, 1H), 3.76 (s, 3H), 3.32 (s, 2H), 2.88-2.76 (m, 3H), 2.72 (s, 4H), 2.64 (d, J=2.4 Hz, 3H), 2.59-2.52 (m, 2H), 2.48-2.34 (m, 3H), 2.24-2.07 (m, 3H), 2.03-1.92 (m, 7H), 1.72-1.39 (m, 8H), 0.72 (s, 3H); [M+H]⁺=961.6.

### Example 7: (R)-3-(4-(3-(4-(1-(4-((5-bromo-4-((5-(dimethylphosphoryl)-2-ethylquinazolin-6-yl)amino)pyrimidin-2-yl)amino)-2-ethyl-5-methoxyphenyl)piperidin-4-yl)piperazin-1-yl)azetidin-1-yl)-2,6-difluorophenyl)piperidine-2,6-dione

### Step 1: 1-(4-(2,6-bis(benzyloxy)pyridin-3-yl)-3,5-difluorophenyl)azetidin-3-ol

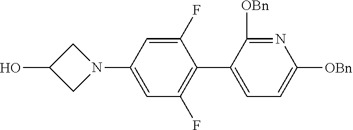 [SMILES: OC1CN(c2cc(F)c(-c3ccc(OCc4ccccc4)nc3OCc3ccccc3)c(F)c2)C1]

A mixture of 2,6-bis(benzyloxy)-3-(4-bromo-2,6-difluorophenyl)pyridine (2 g, 4.16 mmol), azetidin-3-ol hydrochloride (1.36 g, 12.47 mmol), Pd₂(dba)₃ (380 mg, 0.42 mmol), 9,9-dimethyl-4,5-bis(diphenylphosphino)xanthene (486 mg, 0.84 mmol), Cs₂CO₃ (4.07 g, 12.47 mmol) in 1,4-dioxane (40 mL) was stirred in a 100 ml flask at 100° C. under atmosphere of N₂ for 16 h. After being cooled to room temperature, the mixture was filtrated through a pad of celite. The filtrate was concentrated under reduced pressure, the residue was purified by silica gel column eluting with PE/EA (2:1) to give the target product (1.8 g, 91%). [M+H]⁺=475.3.

### Step 2: 3-(2,6-difluoro-4-(3-hydroxyazetidin-1-yl)phenyl)piperidine-2,6-dione

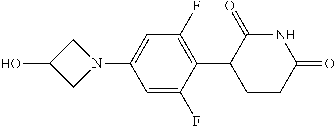 [SMILES: O=C1CCC(c2c(F)cc(N3CC(O)C3)cc2F)C(=O)N1]

A mixture of 1-(4-(2,6-bis(benzyloxy)pyridin-3-yl)-3,5-difluorophenyl)azetidin-3-ol (1.8 g, 3.79 mmol) in iPrOH (30 mL) and DMF (30 mL) was added Pd/C (10%, w/w, 1 g) under N₂(g). The resulting mixture was stirred under H₂ atmosphere for overnight until LC-MS indicated all the starting material was consumed. The resulting solution was filtered and the filtrate was concentrated to give the desire product (0.9 g, 80%). [M+H]⁺=297.2.

### Step 3: 3-(2,6-difluoro-4-(3-oxoazetidin-1-yl)phenyl)piperidine-2,6-dione

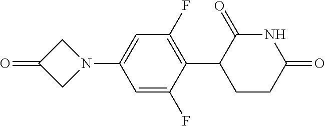 [SMILES: O=C1CN(c2cc(F)c(C3CCC(=O)NC3=O)c(F)c2)C1]

A mixture of 3-(2,6-difluoro-4-(3-hydroxyazetidin-1-yl)phenyl)piperidine-2,6-dione (900 mg, 3.03 mmol) and DMP (1.93 g, 4.55 mmol) in DCM (10 mL) was stirred in a flask at room temperature for 2 hours. The reaction was quenched with con aq Na₂S₂O₃ and the mixture was extracted with DCM, washed three times with saturated aqueous NaCl and twice with saturated aqueous NaHCO₃. The organic layer was dried over anhydrous Na₂SO₄ and evaporated in vacuum to afford the crude product (1.1 g). [M+H]⁺=295.2.

### Step 4: tert-butyl 4-(1-(2-bromo-5-methoxy-4-nitrophenyl)piperidin-4-yl)piperazine-1-carboxylate

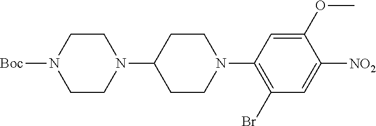 [SMILES: COc1cc(N2CCC(N3CCN(C(=O)OC(C)(C)C)CC3)CC2)c(Br)cc1[N+](=O)[O-]]

A mixture of 1-bromo-2-fluoro-4-methoxy-5-nitrobenzene (4 g, 16 mmol), tert-butyl 4-(piperidin-4-yl)piperazine-1-carboxylate (6.4 g, 24 mmol), K₂CO₃ (4.4 g, 32 mmol) in DMF (50 mL) was stirred in a flask at 80° C. overnight. The reaction mixture was allowed to cool down to room temperature. The resulting mixture was diluted with water and extracted with EtOAc (3×500 mL). The combined organic layers were washed with brine (500 mL), dried over anhydrous Na₂SO₄. After filtration, the filtrate was concentrated under reduced pressure. The residue was purified by silica gel column chromatography, eluted with PE/EtOAc (1:1) to afford the product (7 g, 90%). [M+H]⁺=499.0.

### Step 5: tert-butyl 4-(1-(5-methoxy-4-nitro-2-vinylphenyl)piperidin-4-yl)piperazine-1-carboxylate

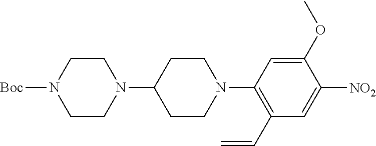 [SMILES: C=Cc1cc([N+](=O)[O-])c(OC)cc1N1CCC(N2CCN(C(=O)OC(C)(C)C)CC2)CC1]

A mixture of tert-butyl 4-(1-(2-bromo-5-methoxy-4-nitrophenyl)piperidin-4-yl)piperazine-1-carboxylate (7 g, 14 mmol), 4,4,5,5-tetramethyl-2-vinyl-1,3,2-dioxaborolane (4.3 g, 28 mmol), Pd(dppf)Cl₂ (1.1 g, 1.4 mmol) and K₃PO₄ (8.9 g, 42 mmol) in DMF (160 mL) and water (20 mL) was stirred in a flask at 90° C. under nitrogen atmosphere for 16 hrs. The reaction mixture was allowed to cool down to room temperature. The resulting mixture was extracted with EtOAc (3×1000 mL). The combined organic layers were washed with brine (500 mL), dried over anhydrous Na₂SO₄. After filtration, the filtrate was concentrated under reduced pressure. The residue was purified by silica gel column chromatography, eluted with PE/EtOAc (1:1) to afford the product (5 g, 80%). [M+H]⁺=447.0.

### Step 6: tert-butyl 4-(1-(4-amino-2-ethyl-5-methoxyphenyl)piperidin-4-yl)piperazine-1-carboxylate

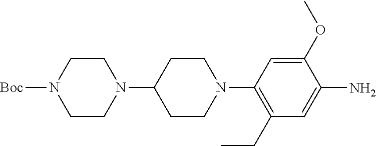 [SMILES: CCc1cc(N)c(OC)cc1N1CCC(N2CCN(C(=O)OC(C)(C)C)CC2)CC1]

To a stirred solution of tert-butyl 4-(1-(5-methoxy-4-nitro-2-vinylphenyl)piperidin-4-yl)piperazine-1-carboxylate (5 g, 11.2 mmol) in MeOH (100 mL) and DCM (20.00 mL) was added Pd/C (wet, 10%) (1 g) under nitrogen atmosphere. The resulting mixture was stirred for 16 hrs at room temperature under hydrogen atmosphere. The resulting mixture was filtered, the filter cake was washed with DCM/CH₃OH (10:1, 200 mL). The filtrate was concentrated under reduced pressure to afford the product (4.0 g, 85.3%). [M+H]⁺=419.1.

### Step 7: (6-((5-bromo-2-((5-ethyl-2-methoxy-4-(4-(piperazin-1-yl)piperidin-1-yl)phenyl)amino)pyrimidin-4-yl)amino)-2-ethylquinazolin-5-yl)dimethylphosphine oxide

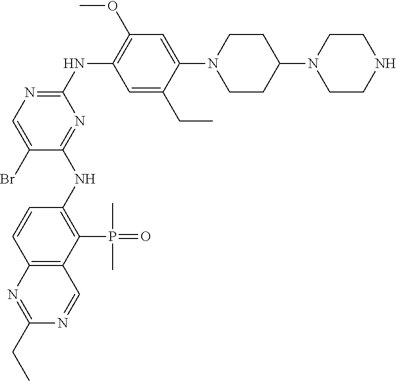 [SMILES: CCc1ncc2c(P(C)(C)=O)c(Nc3nc(Nc4cc(CC)c(N5CCC(N6CCNCC6)CC5)cc4OC)ncc3Br)ccc2n1]

The title compound (210 mg, 45%) was prepared from (6-((5-bromo-2-chloropyrimidin-4-yl)amino)-2-ethylquinazolin-5-yl)dimethylphosphine oxide and tert-butyl 4-(1-(4-amino-2-ethyl-5-methoxyphenyl)piperidin-4-yl)piperazine-1-carboxylate according to the same manner as shown in step 8 of example 1.

### Step 8: (R)-3-(4-(3-(4-(1-(4-((5-bromo-4-((5-(dimethylphosphoryl)-2-ethylquinazolin-6-yl)amino)pyrimidin-2-yl)amino)-2-ethyl-5-methoxyphenyl)piperidin-4-yl)piperazin-1-yl)azetidin-1-yl)-2,6-difluorophenyl)piperidine-2,6-dione

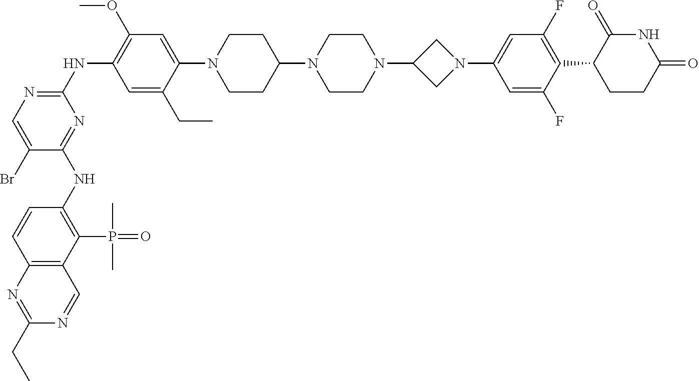 [SMILES: CCc1ncc2c(P(C)(C)=O)c(Nc3nc(Nc4cc(CC)c(N5CCC(N6CCN(C7CN(c8cc(F)c([C@H]9CCC(=O)NC9=O)c(F)c8)C7)CC6)CC5)cc4OC)ncc3Br)ccc2n1]

A mixture of (6-((5-bromo-2-((5-ethyl-2-methoxy-4-(4-(piperazin-1-yl)piperidin-1-yl)phenyl)amino)pyrimidin-4-yl)amino)-2-ethylquinazolin-5-yl)dimethylphosphine oxide (117 mg, 0.16 mmol), 3-(2,6-difluoro-4-(3-oxoazetidin-1-yl)phenyl)piperidine-2,6-dione (95 mg, 0.32 mmol) and NaBH(OAc)₃ (103 mg, 0.49 mmol) and a drop of AcOH was stirred in a 8 ml vial at 70° C. for 2 hours. Then the mixture was evaporated in vacuum to afford the crude product, which was purified with HPLC chromatography (0.1% FA in water:acetonitrile=90:10˜50:50 gradient elution) to give the racemic product (15 mg, 27%). The racemic was purified by SFC ((IF(2*25 cm, 5 um), MtBE (0.1% DEA):(MeOH:DCM=1:1)=60:40, 20 ml/min) and the title compound corresponded to the peak A (4.2 mg, 56%). ¹H NMR (500 MHz, DMSO) δ 11.60 (s, 1H), 10.86 (s, 1H), 9.86 (s, 1H), 8.45 (s, 1H), 8.23 (s, 1H), 8.05 (s, 1H), 7.85 (d, J=9.0 Hz, 1H), 7.27 (s, 1H), 6.74 (s, 1H), 6.12 (d, J=11.1 Hz, 2H), 4.03 (dd, J=12.5, 4.9 Hz, 1H), 3.91 (t, J=7.2 Hz, 2H), 3.75 (s, 3H), 3.64 (s, 2H), 3.25 (d, J=5.3 Hz, 2H), 3.05 (q, J=7.5 Hz, 2H), 2.93 (d, J=10.1 Hz, 2H), 2.77 (dd, J=21.5, 9.1 Hz, 1H), 2.71-2.62 (m, 3H), 2.54 (s, 3H), 2.31 (d, J=50.2 Hz, 7H), 2.03 (d, J=13.4 Hz, 7H), 1.95 (d, J=4.8 Hz, 1H), 1.84 (d, J=10.7 Hz, 2H), 1.54 (d, J=10.3 Hz, 2H), 1.37 (t, J=7.6 Hz, 3H), 0.75 (s, 3H). [M+H]⁺=1000.4.

### Example 8: (R)-3-(4-(3-(4-(1-(4-((5-bromo-4-((5-(dimethylphosphoryl)-3-fluoro-2-methylquinolin-6-yl)amino)pyrimidin-2-yl)amino)-2-ethyl-5-methoxyphenyl)piperidin-4-yl)piperazin-1-yl)azetidin-1-yl)-2,6-difluorophenyl)piperidine-2,6-dione

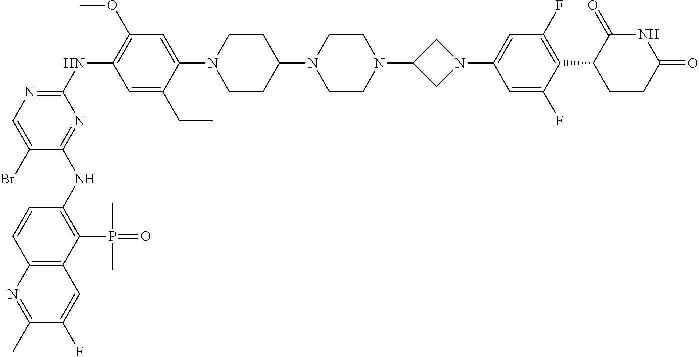 [SMILES: CCc1cc(Nc2ncc(Br)c(Nc3ccc4nc(C)c(F)cc4c3P(C)(C)=O)n2)c(OC)cc1N1CCC(N2CCN(C3CN(c4cc(F)c([C@H]5CCC(=O)NC5=O)c(F)c4)C3)CC2)CC1]

The titled compound was synthesized in the procedures similar to Example 1. ¹H NMR (500 MHz, DMSO) δ 11.61 (s, 1H), 10.86 (s, 1H), 9.85 (s, 1H), 8.45 (s, 1H), 8.23 (s, 1H), 8.05 (s, 1H), 7.85 (d, J=9.2 Hz, 1H), 7.27 (s, 1H), 6.74 (s, 1H), 6.12 (d, J=11.1 Hz, 2H), 4.03 (dd, J=12.5, 4.9 Hz, 1H), 3.91 (t, J=7.2 Hz, 2H), 3.75 (s, 3H), 3.64 (s, 2H), 3.10-3.03 (m, 2H), 3.03-2.82 (m, 3H), 2.82-2.73 (m, 1H), 2.66 (t, J=11.6 Hz, 3H), 2.36 (s, 3H), 2.28-2.06 (m, 4H), 2.03 (d, J=13.4 Hz, 7H), 1.95 (d, J=5.6 Hz, 1H), 1.89 (d, J=43.9 Hz, 2H), 1.83-1.27 (m, 7H), 0.81 (d, J=52.4 Hz, 3H). [M+H]⁺=1003.4.

### Example 9: (R)-3-(4-((S)-3-(4-(1-(4-((5-bromo-4-((5-(dimethylphosphoryl)-2-ethylquinazolin-6-yl)amino)pyrimidin-2-yl)amino)-2-ethyl-5-methoxyphenyl)piperidin-4-yl)piperazin-1-yl)pyrrolidin-1-yl)-2,6-difluorophenyl)piperidine-2,6-dione

### Step 1: (R)-1-(4-(2,6-bis(benzyloxy)pyridin-3-yl)-3,5-difluorophenyl)pyrrolidin-3-ol

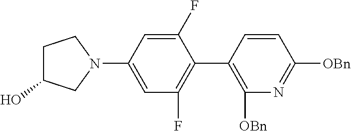 [SMILES: O[C@@H]1CCN(c2cc(F)c(-c3ccc(OCc4ccccc4)nc3OCc3ccccc3)c(F)c2)C1]

To a mixture of 2,6-bis(benzyloxy)-3-(4-bromo-2,6-difluorophenyl)pyridine (5.0 g, 10.39 mmol) in DMA (500 mL) was added (R)-pyrrolidin-3-ol hydrochloride (1.53 g, 12.44 mmol), Pd₂(dba)₃ (0.95 g, 1.03 mmol), BINAP (1.29 g, 2.07 mmol) and Cs₂CO₃ (10.2 g, 31.29 mmol). The mixture was stirred at 100° C. for 16 hours under nitrogen atmosphere. After LCMS showed the reaction was completed. The mixture was diluted with ethyl acetate, washed with brine, dried over anhydrous sodium sulfate, filtered, and concentrated under vacuum. The residue was purified by flash column chromatography with 0-70% ethyl acetate in petroleum ether to afford (R)-1-(4-(2,6-bis(benzyloxy)pyridin-3-yl)-3,5-difluorophenyl)pyrrolidin-3-ol (4.5 g, 90%). [M+H]⁺=489.2.

### Step 2: (R)-1-(4-(2,6-bis(benzyloxy)pyridin-3-yl)-3,5-difluorophenyl)pyrrolidin-3-yl methanesulfonate

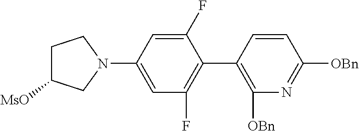 [SMILES: CS(=O)(=O)O[C@@H]1CCN(c2cc(F)c(-c3ccc(OCc4ccccc4)nc3OCc3ccccc3)c(F)c2)C1]

To a mixture of (R)-1-(4-(2,6-bis(benzyloxy)pyridin-3-yl)-3,5-difluorophenyl)pyrrolidin-3-ol (1.0 g, 2.05 mmol) and TEA (0.62 g, 6.15 mmol) in DCM (10 mL) was added MsCl (0.35 g, 3.07 mmol) dropwise at 0° C. The mixture was stirred at room temperature for 2 hours. After LCMS showed the reaction was completed. The mixture was diluted with DCM, washed with brine, dried over anhydrous sodium sulfate, filtered, and concentrated under vacuum. The residue was purified by flash column chromatography with 0-80% ethyl acetate in petroleum ether to afford (R)-1-(4-(2,6-bis(benzyloxy)pyridin-3-yl)-3,5-difluorophenyl)pyrrolidin-3-yl methanesulfonate (1.0 g, 86%). [M+H]⁺=567.2.

### Step 3: benzyl(S)-4-(1-(4-(2,6-bis(benzyloxy)pyridin-3-yl)-3,5-difluorophenyl)pyrrolidin-3-yl)piperazine-1-carboxylate

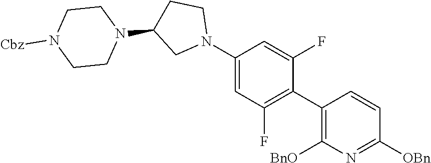 [SMILES: O=C(OCc1ccccc1)N1CCN([C@H]2CCN(c3cc(F)c(-c4ccc(OCc5ccccc5)nc4OCc4ccccc4)c(F)c3)C2)CC1]

To a mixture of (R)-1-(4-(2,6-bis(benzyloxy)pyridin-3-yl)-3,5-difluorophenyl)pyrrolidin-3-yl methanesulfonate (0.90 g, 1.59 mmol) and DIEA (0.61 g, 4.77 mmol) in ACN (10 mL) was added benzyl piperazine-1-carboxylate (1.1 g, 4.77 mmol). The mixture was stirred at 100° C. for 16 hours. After LCMS showed the reaction was completed. The mixture was diluted with DCM, washed with brine, dried over anhydrous sodium sulfate, filtered, and concentrated under vacuum. The residue was purified by flash column chromatography with 0-80% ethyl acetate in petroleum ether to afford benzyl (S)-4-(1-(4-(2,6-bis(benzyloxy)pyridin-3-yl)-3,5-difluorophenyl)pyrrolidin-3-yl)piperazine-1-carboxylate (0.72 g, 59%). [M+H]⁺=691.3.

### Step 4: 3-(2,6-difluoro-4-((S)-3-(piperazin-1-yl)pyrrolidin-1-yl)phenyl)piperidine-2,6-dione

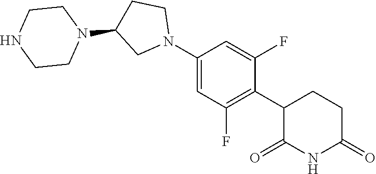 [SMILES: O=C1CCC(c2c(F)cc(N3CC[C@H](N4CCNCC4)C3)cc2F)C(=O)N1]

To the solution of benzyl (S)-4-(1-(4-(2,6-bis(benzyloxy)pyridin-3-yl)-3,5-difluorophenyl)pyrrolidin-3-yl)piperazine-1-carboxylate (0.72 g, 1.04 mmol) in AcOH (20 mL) was added Pd/C (0.72 g, 10 wt. %, wet). The mixture was stirred at 50° C. for 16 hours under hydrogen atmosphere (balloon). After LCMS showed the reaction was completed, the mixture was cooled to room temperature and filtered by celite directly. The filtrate was concentrated in vacuum to afford the crude product (377 mg). [M+H]⁺=379.2.

### Step 5: 8-(5-methoxy-4-nitro-2-vinylphenyl)-1,4-dioxa-8-azaspiro[4.5]decane

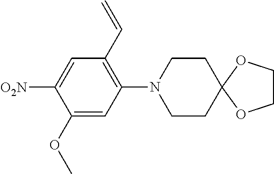 [SMILES: C=Cc1cc([N+](=O)[O-])c(OC)cc1N1CCC2(CC1)OCCO2]

To a mixture of 1-fluoro-5-methoxy-4-nitro-2-vinylbenzene (10 g, 50.8 mmol) and 1,4-dioxa-8-azaspiro[4.5]decane (8.7 g, 60.9 mmol) in CH₃CN (100 mL) was added K₂CO₃ (14 g, 101.6 mmol). Then the mixture was stirred at 70° C. for 18 hrs. Under cooling with ice, the reaction was quenched by water (100 mL) and the resulting mixture was extracted with EtOAc (100 mL). The combined organic phase was washed with brine (40 mL), dried over Na₂SO₄, filtered and concentrated in vacuum. The residue was purified by silica gel column (PE:EA=2:1). 8-(5-methoxy-4-nitro-2-vinylphenyl)-1,4-dioxa-8-azaspiro[4.5]decane (14.8 g, 90.8%) was obtained. [M+H]⁺=321.2.

### Step 6: 5-ethyl-2-methoxy-4-(1,4-dioxa-8-azaspiro[4.5]decan-8-yl)aniline

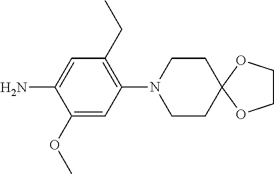 [SMILES: CCc1cc(N)c(OC)cc1N1CCC2(CC1)OCCO2]

To a solution of 8-(5-methoxy-4-nitro-2-vinylphenyl)-1,4-dioxa-8-azaspiro[4.5]decane (13 g, 40.6 mmol) in iPrOH (15 mL) and DCM (80 mL) was added Pd/C (2.3 g, 10%), the resulting mixture was stirred at r.t under H₂ balloon for 5 hrs. The solid was filtered off, the filtrate was concentrated in vacuo. The residue was purified by silica gel column (PE:EA=1:1). 5-ethyl-2-methoxy-4-(1,4-dioxa-8-azaspiro[4.5]decan-8-yl)aniline (10.2 g, 85.7%) was obtained. [M+H]⁺=293.3.

### Step 7: (6-((5-bromo-2-((5-ethyl-2-methoxy-4-(1,4-dioxa-8-azaspiro[4.5]decan-8-yl)phenyl)amino)pyrimidin-4-yl)amino)-2-ethylquinazolin-5-yl)dimethylphosphine oxide

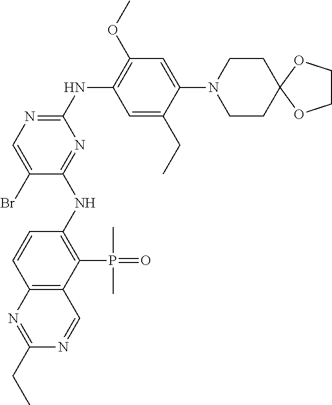 [SMILES: CCc1ncc2c(P(C)(C)=O)c(Nc3nc(Nc4cc(CC)c(N5CCC6(CC5)OCCO6)cc4OC)ncc3Br)ccc2n1]

To a mixture of (6-((5-bromo-2-chloropyrimidin-4-yl)amino)-2-ethylquinazolin-5-yl)dimethylphosphine oxide (5.2 g, 11.4 mmol) and 5-ethyl-2-methoxy-4-(1,4-dioxa-8-azaspiro[4.5]decan-8-yl)aniline (4.3 g, 14.9 mmol) in ethylene glycol (52 mL) was added TsOH (2.94 g, 17.1 mmol). The mixture was stirred at 80° C. for 18 hrs. Under cooling with ice, the reaction was quenched by sat NaHCO₃ solution (40 mL) and the resulting mixture was extracted with EtOAc (50 mL×3). The combined organic phase was washed with brine (40 mL), dried over Na₂SO₄, filtered and concentrated in vacuum. The residue was purified by silica gel column (DCM:CH₃OH=15:1). (6-((5-bromo-2-((5-ethyl-2-methoxy-4-(1,4-dioxa-8-azaspiro[4.5]decan-8-yl)phenyl)amino)pyrimidin-4-yl)amino)-2-ethylquinazolin-5-yl)dimethylphosphine oxide (4.6 g, 58.2%) was obtained. [M+H]⁺=696.2.

### Step 8: 1-(4-((5-bromo-4-((5-(dimethylphosphoryl)-2-ethylquinazolin-6-yl)amino)pyrimidin-2-yl)amino)-2-ethyl-5-methoxyphenyl)piperidin-4-one

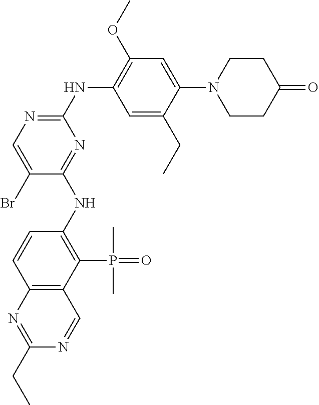 [SMILES: CCc1ncc2c(P(C)(C)=O)c(Nc3nc(Nc4cc(CC)c(N5CCC(=O)CC5)cc4OC)ncc3Br)ccc2n1]

To a mixture of (6-((5-bromo-2-((5-ethyl-2-methoxy-4-(1,4-dioxa-8-azaspiro[4.5]decan-8-yl)phenyl)amino)pyrimidin-4-yl)amino)-2-ethylquinazolin-5-yl)dimethylphosphine oxide (4 g, 5.75 mmol) in THF (30 mL) and H₂O (30 mL) was added TsOH (1.98 g, 11.5 mmol). The mixture was stirred at 70° C. for 18 hrs. Under cooling with ice, the reaction was quenched by sat NaHCO₃ solution (20 mL) and the resulting mixture was extracted with EtOAc (30 mL×3). The combined organic phase was washed with brine (40 mL), dried over Na₂SO₄, filtered and concentrated in vacuum. 1-(4-((5-bromo-4-((5-(dimethylphosphoryl)-2-ethylquinazolin-6-yl)amino)pyrimidin-2-yl)amino)-2-ethyl-5-methoxyphenyl)piperidin-4-one (3.4 g, 90.7%) was obtained. [M+H]⁺=652.1.

### Step 9: (R)-3-(4-((S)-3-(4-(1-(4-((5-bromo-4-((5-(dimethylphosphoryl)-2-ethylquinazolin-6-yl)amino)pyrimidin-2-yl)amino)-2-ethyl-5-methoxyphenyl)piperidin-4-yl)piperazin-1-yl)pyrrolidin-1-yl)-2,6-difluorophenyl)piperidine-2,6-dione

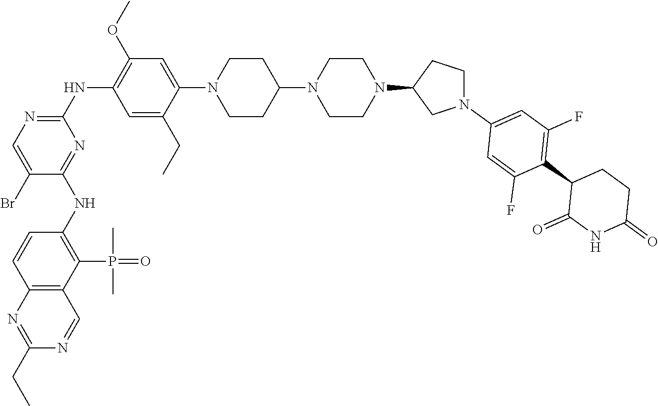 [SMILES: CCc1ncc2c(P(C)(C)=O)c(Nc3nc(Nc4cc(CC)c(N5CCC(N6CCN([C@H]7CCN(c8cc(F)c([C@H]9CCC(=O)NC9=O)c(F)c8)C7)CC6)CC5)cc4OC)ncc3Br)ccc2n1]

To the solution of 1-(4-((5-bromo-4-((5-(dimethylphosphoryl)-2-ethylquinazolin-6-yl)amino)pyrimidin-2-yl)amino)-2-ethyl-5-methoxyphenyl)piperidin-4-one (86 mg, 0.13 mmol) in DCE (2 mL) was added 3-(2,6-difluoro-4-((S)-3-(piperazin-1-yl)pyrrolidin-1-yl)phenyl)piperidine-2,6-dione (50 mg, 0.13 mmol), DIEA (119 mg, 0.92 mmol) and NaBH(OAc)₃ (112 mg, 0.53 mmol). The mixture was stirred at 70° C. for 16 hours.

After LCMS showed the reaction was completed. The mixture was diluted with DCM, washed with brine, dried over anhydrous sodium sulfate, filtered, and concentrated under vacuum. The residue was purified by flash column chromatography with 0-25% MeOH in DCM and Prep-HPLC chromatography (0.1% FA in water:acetonitrile=90:10˜50:50 gradient elution) to afford 3-(4-((S)-3-(4-(1-(4-((5-bromo-4-((5-(dimethylphosphoryl)-2-ethylquinazolin-6-yl)amino)pyrimidin-2-yl)amino)-2-ethyl-5-methoxyphenyl)piperidin-4-yl)piperazin-1-yl)pyrrolidin-1-yl)-2,6-difluorophenyl)piperidine-2,6-dione which was further purified by SFC (IF (2*25 cm, 5 um), 60% MtBE (0.1% DEA)/40% MeOH:DCM (1:1), 100 bar, 20 ml/min) and the title compound corresponded to peak A @ 1.711 min/254 nm (14 mg, 28%). ¹H NMR (500 MHz, DMSO) δ 11.62 (s, 1H), 10.84 (s, 1H), 9.85 (s, 1H), 8.45 (s, 1H), 8.23 (s, 1H), 8.05 (s, 1H), 7.85 (d, J=9.0 Hz, 1H), 7.27 (s, 1H), 6.75 (s, 1H), 6.23 (d, J=12.5 Hz, 2H), 4.05-3.98 (m, 1H), 3.75 (s, 3H), 3.50-3.42 (m, 1H), 3.39-3.34 (m, 1H), 3.30-3.26 (m, 1H), 3.25-3.16 (m, 1H), 3.10-2.99 (m, 3H), 2.99-2.51 (m, 13H), 2.34-1.70 (m, 16H), 1.62-1.46 (m, 2H), 1.38 (t, J=7.5 Hz, 3H), 0.76 (s, 3H); [M+H]⁺=1014.5.

### Example 10: (R)-3-(4-(4-(4-(1-(4-((5-bromo-4-((5-(dimethylphosphoryl)-2-ethylquinolin-6-yl)amino)pyrimidin-2-yl)amino)-2-ethyl-5-methoxyphenyl)piperidin-4-yl)piperazin-1-yl)piperidin-1-yl)-2,6-difluorophenyl)piperidine-2,6-dione

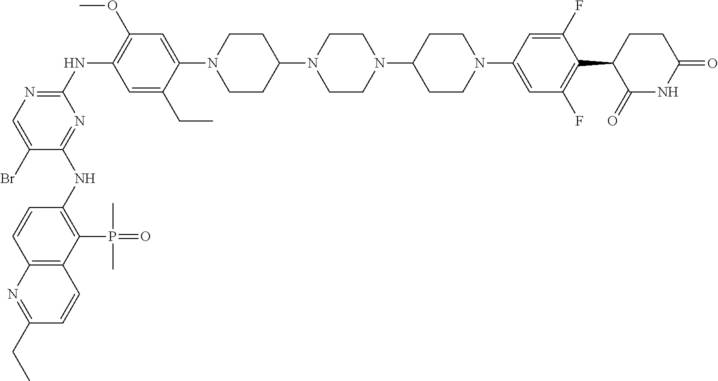 [SMILES: CCc1ccc2c(P(C)(C)=O)c(Nc3nc(Nc4cc(CC)c(N5CCC(N6CCN(C7CCN(c8cc(F)c([C@H]9CCC(=O)NC9=O)c(F)c8)CC7)CC6)CC5)cc4OC)ncc3Br)ccc2n1]

To the solution of (6-((5-bromo-2-((5-ethyl-2-methoxy-4-(4-(piperazin-1-yl)piperidin-1-yl)phenyl)amino)pyrimidin-4-yl)amino)-2-ethylquinolin-5-yl)dimethylphosphine oxide (the intermediate was obtained through the similar way of Example 7 step 7) (70 mg, 0.1 mmol) and 3-(2,6-difluoro-4-(4-oxopiperidin-1-yl)phenyl)piperidine-2,6-dione (40 mg, 0.13 mmol) in 3 mL DCE, 1 drop of Ti(OiPr)₄ was added. The mixture was stirred at 40° C. for 2 hours. Then, NaBH(OAc)₃ (40 mg, 0.2 mmol) was added. The mixture was stirred at 40° C. for 1 hour. The mixture was quenched with NaHCO₃ aqueous and filtered. The organic layer was concentrated in vacuum and purified by prep-TLC (DCM:MeOH=11:1). The crude product was purified by SFC (IF (2 cm×25 cm, 5 um), MtBE (0.1% DEA):(MeOH:DCM=1:1)=40:60, 100 bar, 20 ml/min) and the title compound corresponded to peak A @ 1.342 min/254 nm (12 mg, 12% yield). ¹H NMR (500 MHz, DMSO) δ 11.79 (s, 1H), 10.87 (s, 1H), 8.56 (d, J=9.2 Hz, 1H), 8.28 (s, 1H), 8.21 (s, 1H), 8.00 (s, 1H), 7.87 (d, J=9.0 Hz, 1H), 7.44 (d, J=9.0 Hz, 1H), 7.33 (s, 1H), 6.75 (s, 1H), 6.63 (d, J=13.4 Hz, 2H), 4.04 (dd, J=13.1, 5.3 Hz, 1H), 3.82-3.74 (m, 5H), 3.31-3.27 (m, 2H), 2.96-2.89 (m, 4H), 2.83-2.62 (m, 7H), 2.60-2.54 (m, 6H), 2.41-2.23 (m, 4H), 2.14-2.04 (m, 1H), 2.03-1.93 (m, 7H), 1.89-1.79 (m, 3H), 1.59-1.38 (m, 4H), 1.32 (t, J=7.6 Hz, 3H), 0.77 (s, 3H); [M+H]⁺=1027.7.

### Example 11: (R)-3-(4-(4-(1′-(4-((5-bromo-4-((5-(dimethylphosphoryl)-2-ethylquinolin-6-yl)amino)pyrimidin-2-yl)amino)-2-ethyl-5-methoxyphenyl)-[1,4′-bipiperidin]-4-yl)piperazin-1-yl)-2,6-difluorophenyl)piperidine-2,6-dione

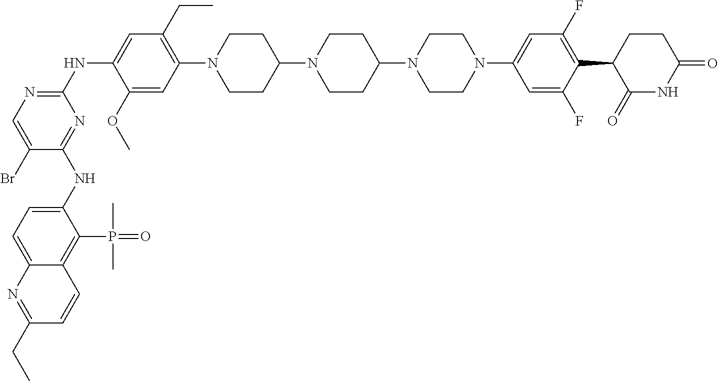 [SMILES: CCc1ccc2c(P(C)(C)=O)c(Nc3nc(Nc4cc(CC)c(N5CCC(N6CCC(N7CCN(c8cc(F)c([C@H]9CCC(=O)NC9=O)c(F)c8)CC7)CC6)CC5)cc4OC)ncc3Br)ccc2n1]

A mixture of 1′-(4-((5-bromo-4-((5-(dimethylphosphoryl)-2-ethylquinolin-6-yl)amino)pyrimidin-2-yl)amino)-2-ethyl-5-methoxyphenyl)-[1,4′-bipiperidin]-4-one (the intermediate was obtained through the similar way of Example 7 step 7) (70 mg, 0.1 mmol), (R)-3-(2,6-difluoro-4-(piperazin-1-yl)phenyl)piperidine-2,6-dione (the intermediate was obtained through the similar way of Example 9 step 4) (29 mg, 0.1 mmol), STAB (40 mg, 0.2 mmol) and Ti(i-PrOH)₄ (27 mg, 0.1 mmol) in DMSO (10 mL) was stirred in a flask at 80° C. overnight. The reaction mixture was allowed to cool down to room temperature. The resulting mixture was diluted with DCM (100 mL), washed with brine (100 mL), dried over anhydrous Na₂SO₄. After filtration, the filtrate was concentrated under reduced pressure. The residue was purified by silica gel column chromatography (DCM:MEOH=0-10%) to afford the crude product which was purified by SFC (IH (3*25 cm, 5 um), 13% EtOH/87% CO₂, 100 bar, 100 ml/min) and the title compound corresponded to peak A @ 1.24 min/254 nm (8.9 mg, 9%). ¹H NMR (500 MHz, DMSO) δ 11.80 (s, 1H), 10.87 (s, 1H), 8.55 (d, J=8.4 Hz, 1H), 8.27 (s, 2H), 8.00 (s, 1H), 7.87 (d, J=9.0 Hz, 1H), 7.44 (d, J=8.9 Hz, 1H), 7.33 (s, 1H), 6.75 (s, 1H), 6.63 (d, J=12.9 Hz, 2H), 4.05 (d, J=8.2 Hz, 1H), 3.76 (s, 3H), 3.17 (s, 4H), 3.06-2.87 (m, 6H), 2.77 (d, J=11.9 Hz, 1H), 2.67 (t, J=11.8 Hz, 2H), 2.59 (s, 4H), 2.28 (s, 3H), 2.17 (s, 1H), 1.98 (d, J=13.2 Hz, 6H), 1.79 (s, 5H), 1.57 (d, J=10.8 Hz, 4H), 1.40 (d, J=9.5 Hz, 2H), 1.32 (t, J=7.5 Hz, 4H), 0.77 (s, 4H). [M+H]⁺=1027.8.

### Example 12: (R)-3-(4-(4-(1′-(4-((5-chloro-4-((5-(dimethylphosphoryl)-3-fluoro-2-methylquinolin-6-yl)amino)pyrimidin-2-yl)amino)-2-ethyl-5-methoxyphenyl)-[1,4′-bipiperidin]-4-yl)piperazin-1-yl)-2,6-difluorophenyl)piperidine-2,6-dione

### Step 1: (6-((5-chloro-2-((5-ethyl-2-methoxy-4-(1,4-dioxa-8-azaspiro[4.5]decan-8-yl)phenyl)amino)pyrimidin-4-yl)amino)-3-fluoro-2-methylquinolin-5-yl)dimethylphosphine oxide

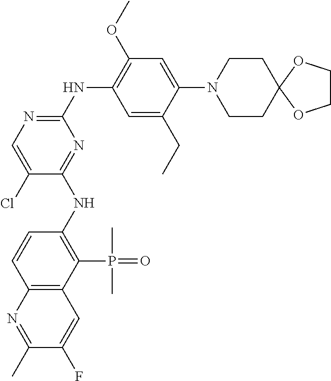 [SMILES: CCc1cc(Nc2ncc(Cl)c(Nc3ccc4nc(C)c(F)cc4c3P(C)(C)=O)n2)c(OC)cc1N1CCC2(CC1)OCCO2]

To a solution of (6-((2,5-dichloropyrimidin-4-yl)amino)-3-fluoro-2-methylquinolin-5-yl)dimethylphosphine oxide (1.35 g, 3.4 mmol) and 5-ethyl-2-methoxy-4-(1,4-dioxa-8-azaspiro[4.5]decan-8-yl)aniline (1.2 g, 4.1 mmol) in ethylene glycol (50 mL) and CF₃CH₂OH (25 mL) was added TsOH (1.0 g, 5.9 mmol) at room temperature. The resulting mixture was stirred at 80° C. for 48 hours. The reaction was concentrated, basified with NaHCO₃ aqueous, extracted by DCM (2×60 mL). The combined organic layers were washed with brine (2×50 mL), dried over Na₂SO₄, filtered and concentrated in vacuum to give the crude residue, which was purified by silica gel column chromatography (DCM:MeOH=100:0˜20:1 gradient elution) to afford the desired product (1.87 g, 84%). [M+H]⁺=655.3.

### Step 2: 1-(4-((5-chloro-4-((5-(dimethylphosphoryl)-3-fluoro-2-methylquinolin-6-yl)amino)pyrimidin-2-yl)amino)-2-ethyl-5-methoxyphenyl)piperidin-4-one

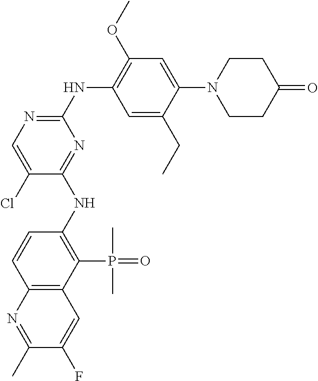 [SMILES: CCc1cc(Nc2ncc(Cl)c(Nc3ccc4nc(C)c(F)cc4c3P(C)(C)=O)n2)c(OC)cc1N1CCC(=O)CC1]

To a solution of (6-((5-chloro-2-((5-ethyl-2-methoxy-4-(1,4-dioxa-8-azaspiro[4.5]decan-8-yl)phenyl)amino)pyrimidin-4-yl)amino)-3-fluoro-2-methylquinolin-5-yl)dimethylphosphine oxide (1.9 g, 2.9 mmol) in THF (45 mL) and H₂O (40 mL) was added TsOH (1.0 g, 5.8 mmol) at room temperature. The resulting mixture was heated at 70° C. overnight. The reaction was concentrated, basified with NaHCO₃ aqueous, extracted by DCM (2×80 mL). The combined organic layers were washed with brine (lx 60 mL), dried over Na₂SO₄, filtered and concentrated in vacuum to give the desired product (1.7 g, 96%). [M+H]⁺=611.1.

### Step 3: (6-((2-((4-(4-(1,4-dioxa-8-azaspiro[4.5]decan-8-yl)piperidin-1-yl)-5-ethyl-2-methoxyphenyl)amino)-5-chloropyrimidin-4-yl)amino)-3-fluoro-2-methylquinolin-5-yl)dimethylphosphine oxide

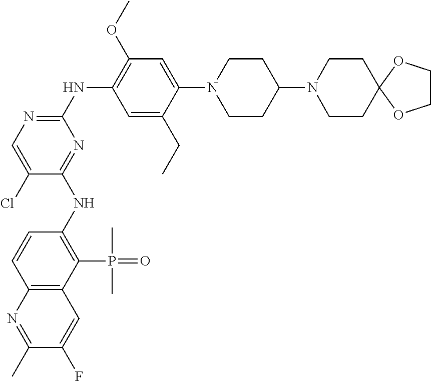 [SMILES: CCc1cc(Nc2ncc(Cl)c(Nc3ccc4nc(C)c(F)cc4c3P(C)(C)=O)n2)c(OC)cc1N1CCC(N2CCC3(CC2)OCCO3)CC1]

To a solution of 1-(4-((5-chloro-4-((5-(dimethylphosphoryl)-3-fluoro-2-methylquinolin-6-yl)amino)pyrimidin-2-yl)amino)-2-ethyl-5-methoxyphenyl)piperidin-4-one (465 mg, 0.76 mmol) and 1,4-dioxa-8-azaspiro[4.5]decane (328 mg, 2.28 mmol) in DMSO (50 mL) was added NaBH(OAc)₃ (486 mg, 2.28 mmol) at room temperature. The resulting mixture was heated at 80° C. for 4 hours. The reaction was quenched with water and extracted by DCM (2×60 mL). The combined organic layers were washed with brine (2×50 mL), dried over Na₂SO₄, filtered and concentrated in vacuum to give the crude residue, which was purified by silica gel column chromatography (DCM:MeOH=100:0˜15:1 gradient elution) to afford the desired product (448 mg, 80%). [M+H]⁺=738.4.

### Step 4: 1′-(4-((5-chloro-4-((5-(dimethylphosphoryl)-3-fluoro-2-methylquinolin-6-yl)amino)pyrimidin-2-yl)amino)-2-ethyl-5-methoxyphenyl)-[1,4′-bipiperidin]-4-one

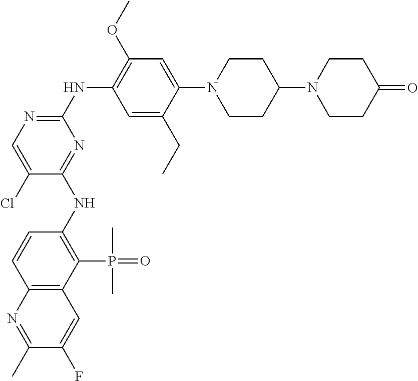 [SMILES: CCc1cc(Nc2ncc(Cl)c(Nc3ccc4nc(C)c(F)cc4c3P(C)(C)=O)n2)c(OC)cc1N1CCC(N2CCC(=O)CC2)CC1]

A solution of (6-((2-((4-(4-(1,4-dioxa-8-azaspiro[4.5]decan-8-yl)piperidin-1-yl)-5-ethyl-2-methoxyphenyl)amino)-5-chloropyrimidin-4-yl)amino)-3-fluoro-2-methylquinolin-5-yl)dimethylphosphine oxide (450 mg, 0.61 mmol) in 8N HCl (12 mL) was stirred at room temperature overnight. The reaction was basified with Na₂CO₃ aqueous, extracted by DCM (2×80 mL). The combined organic layers were washed with brine (lx 60 mL), dried over Na₂SO₄, filtered and concentrated in vacuum to give the desired product (393 mg, 93%). [M+H]⁺=694.2.

### Step 5: (R)-3-(4-(4-(1′-(4-((5-chloro-4-((5-(dimethylphosphoryl)-3-fluoro-2-methylquinolin-6-yl)amino)pyrimidin-2-yl)amino)-2-ethyl-5-methoxyphenyl)-[1,4′-bipiperidin]-4-yl)piperazin-1-yl)-2,6-difluorophenyl)piperidine-2,6-dione

To a solution of 1′-(4-((5-chloro-4-((5-(dimethylphosphoryl)-3-fluoro-2-methylquinolin-6-yl)amino)pyrimidin-2-yl)amino)-2-ethyl-5-methoxyphenyl)-[1,4′-bipiperidin]-4-one (69 mg, 0.1 mmol) and (R)-3-(2,6-difluoro-4-(piperazin-1-yl)phenyl)piperidine-2,6-dione (38 mg, 0.12 mmol) in DCE (5 mL) was added STAB (43 mg, 0.2 mmol) at 60° C. The mixture was stirred at 60° C. for 1 hr. Water (10 mL) was poured into the mixture. Then the mixture was extracted with DCM (20 mL). The organic phase was washed with brine (10 mL), dried over Na₂SO₄, filtered and concentrated in vacuum. The residue was purified by prep-HPLC (C-18 column chromatography (0.1% FA in water:acetonitrile=90:10˜60:40 gradient elution) to afford the product (21.2 mg, 21.5%). ¹H NMR (500 MHz, DMSO) δ 11.65 (s, 1H), 10.87 (s, 1H), 8.56 (d, J=12.1 Hz, 1H), 8.35 (s, 1H), 8.15 (s, 1H), 7.98 (s, 1H), 7.94 (d, J=9.1 Hz, 1H), 7.35 (s, 1H), 6.73 (s, 1H), 6.64 (s, 1H), 6.61 (s, 1H), 4.05 (dd, J=12.5, 4.7 Hz, 1H), 3.75 (s, 3H), 3.31-3.27 (m, 2H), 3.17 (s, 4H), 3.05-2.89 (m, 4H), 2.78 (t, J=12.6 Hz, 1H), 2.69-2.55 (m, 10H), 2.29-2.04 (m, 6H), 1.98 (s, 3H), 1.95 (s, 3H), 1.87-1.74 (m, 4H), 1.66-1.53 (m, 2H), 1.41 (d, J=9.5 Hz, 2H), 0.77 (s, 3H). [M+H]⁺=987.7

### Example 13: (R)-3-(4-(1-(1-(1-(4-((5-bromo-4-((5-(dimethylphosphoryl)-2-ethylquinazolin-6-yl)amino)pyrimidin-2-yl)amino)-2-ethyl-5-methoxyphenyl)piperidin-4-yl)azetidin-3-yl)piperidin-4-yl)-2,6-difluorophenyl)piperidine-2,6-dione

### Step 1: tert-butyl 4-(4-(2,6-bis(benzyloxy)pyridin-3-yl)-3,5-difluorophenyl)-3,6-dihydropyridine-1(2H)-carboxylate

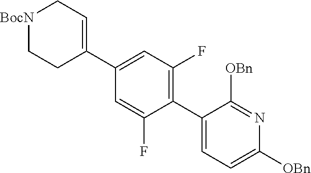 [SMILES: CC(C)(C)OC(=O)N1CC=C(c2cc(F)c(-c3ccc(OCc4ccccc4)nc3OCc3ccccc3)c(F)c2)CC1]

A mixture of 2,6-bis(benzyloxy)-3-(4-bromo-2,6-difluorophenyl)pyridine (4.81 g, 10.0 mmol), Pd(dppf)Cl₂ (731 mg, 1 mmol), tert-butyl 4-(4,4,5,5-tetramethyl-1,3-dioxolan-2-yl)-3,6-dihydropyridine-1(2H)-carboxylate (3.42 g, 11.0 mmol), and K₂CO₃ (2.76 g, 20.0 mmol) in dioxane (80 mL) and H₂O (20 mL) was stirred in a flask at 100° C. for 2 hrs under N₂. Water (160 mL) was poured into the mixture, extracted with EA (200 mL). The combined organic phase was washed with brine (100 mL), dried over Na₂SO₄, filtered and concentrated in vacuum. The residue was purified by column chromatography. tert-butyl 4-(4-(2,6-bis(benzyloxy)pyridin-3-yl)-3,5-difluorophenyl)-3,6-dihydropyridine-1(2H)-carboxylate (5.5 g, 94.0%) was obtained. [M+H]⁺=585.2.

### Step 2: 2,6-bis(benzyloxy)-3-(2,6-difluoro-4-(1,2,3,6-tetrahydropyridin-4-yl)phenyl)pyridine

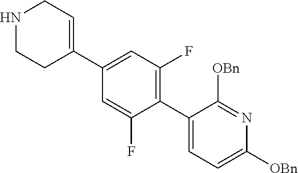 [SMILES: Fc1cc(C2=CCNCC2)cc(F)c1-c1ccc(OCc2ccccc2)nc1OCc1ccccc1]

A mixture of tert-butyl 4-(4-(2,6-bis(benzyloxy)pyridin-3-yl)-3,5-difluorophenyl)-3,6-dihydropyridine-1(2H)-carboxylate (2.34 g, 4.0 mmol) in HCl (10 mL, 4N) was stirred in a flask at RT for 2 hrs. The mixture was concentrated in vacuum, extracted with EA (200 mL), washed with NaHCO₃ (aq, 100 ml), dried over Na₂SO₄, filtered and concentrated in vacuum. 2,6-bis(benzyloxy)-3-(2,6-difluoro-4-(1,2,3,6-tetrahydropyridin-4-yl)phenyl)pyridine (1.9 g, 98.1%) was obtained. [M+H]⁺=485.2.

### Step 3: tert-butyl 3-(4-(4-(2,6-bis(benzyloxy)pyridin-3-yl)-3,5-difluorophenyl)-3,6-dihydropyridin-1(2H)-yl)azetidine-1-carboxylate

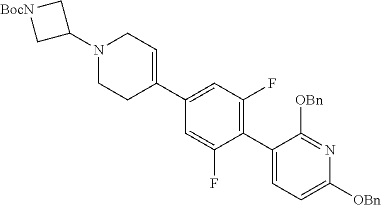 [SMILES: CC(C)(C)OC(=O)N1CC(N2CC=C(c3cc(F)c(-c4ccc(OCc5ccccc5)nc4OCc4ccccc4)c(F)c3)CC2)C1]

To a solution of 2,6-bis(benzyloxy)-3-(2,6-difluoro-4-(1,2,3,6-tetrahydropyridin-4-yl)phenyl)pyridine (1.45 g, 3.0 mmol) and tert-butyl 3-oxoazetidine-1-carboxylate (564 mg, 3.3 mmol) in DCE (30 mL) was added STAB (1.27 g, 6.0 mmol) at 60° C. The mixture was stirred at 20° C. for 1 hr. Water (50 mL) was poured into the mixture. Then the mixture was extracted with DCM (100 mL). The organic phase was washed with brine (50 mL), dried over Na₂SO₄, filtered and concentrated in vacuum. The residue was purified by column chromatography. tert-butyl 3-(4-(4-(2,6-bis(benzyloxy)pyridin-3-yl)-3,5-difluorophenyl)-3,6-dihydropyridin-1(2H)-yl)azetidine-1-carboxylate (1.4 g, 72.9%) was obtained. [M+H]⁺=640.3.

### Step 4: tert-butyl 3-(4-(4-(2,6-dioxopiperidin-3-yl)-3,5-difluorophenyl)piperidin-1-yl)azetidine-1-carboxylate

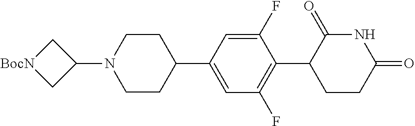 [SMILES: CC(C)(C)OC(=O)N1CC(N2CCC(c3cc(F)c(C4CCC(=O)NC4=O)c(F)c3)CC2)C1]

To a mixture of tert-butyl 3-(4-(4-(2,6-bis(benzyloxy)pyridin-3-yl)-3,5-difluorophenyl)-3,6-dihydropyridin-1(2H)-yl)azetidine-1-carboxylate (1.4 g, 2.2 mmol) in DMF (20 mL) and i-PrOH (5 mL) added Pd/C (700 mg, 10%). The resulting mixture was stirred at 50° C. under H₂ for 16 hrs. The reaction mixture was filtered, the filtrate was concentrated in vacuo to afford tert-butyl 3-(4-(4-(2,6-dioxopiperidin-3-yl)-3,5-difluorophenyl)piperidin-1-yl)azetidine-1-carboxylate (0.91 g, 89.0%). [M+H]⁺=464.3.

### Step 5: (R)-3-(4-(1-(azetidin-3-yl)piperidin-4-yl)-2,6-difluorophenyl)piperidine-2,6-dione

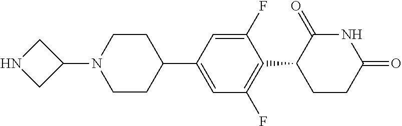 [SMILES: O=C1CC[C@H](c2c(F)cc(C3CCN(C4CNC4)CC3)cc2F)C(=O)N1]

A mixture of t tert-butyl 3-(4-(4-(2,6-dioxopiperidin-3-yl)-3,5-difluorophenyl)piperidin-1-yl)azetidine-1-carboxylate (0.91 g, 1.9 mmol) in HCl (10 mL, 4N) was stirred in a flask at RT for 2 hrs. The mixture was concentrated in vacuum to afford the crude product which was purified by SFC (IH (3*25 cm, 5 um), 13% EtOH/87% CO₂, 100 bar, 100 ml/min) and the title compound corresponded to peak A @ 0.655 min/254 nm (200 mg, 34%). [M+H]⁺=364.2.

### Step 6: (R)-3-(4-(1-(1-(1-(4-((5-bromo-4-((5-(dimethylphosphoryl)-2-ethylquinazolin-6-yl)amino)pyrimidin-2-yl)amino)-2-ethyl-5-methoxyphenyl)piperidin-4-yl)azetidin-3-yl)piperidin-4-yl)-2,6-difluorophenyl)piperidine-2,6-dione

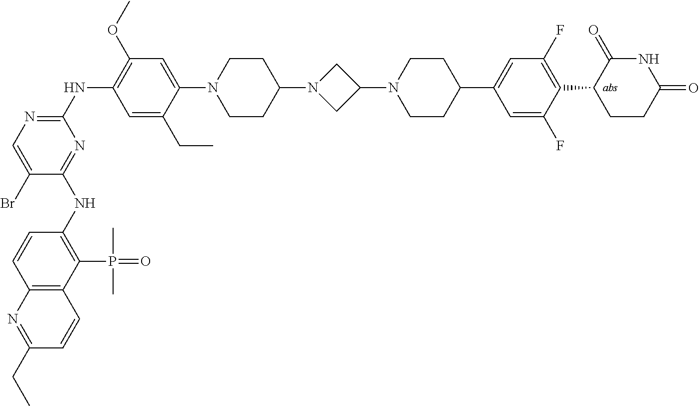 [SMILES: CCc1ccc2c(P(C)(C)=O)c(Nc3nc(Nc4cc(CC)c(N5CCC(N6CC(N7CCC(c8cc(F)c([C@H]9CCC(=O)NC9=O)c(F)c8)CC7)C6)CC5)cc4OC)ncc3Br)ccc2n1]

The titled compound (19 mg, 30%) was prepared in a manner similar to that in Example 9 step 9 from 1-(4-((5-bromo-4-((5-(dimethylphosphoryl)-2-ethylquinazolin-6-yl)amino)pyrimidin-2-yl)amino)-2-ethyl-5-methoxyphenyl)piperidin-4-one and (R)-3-(4-(1-(azetidin-3-yl)piperidin-4-yl)-2,6-difluorophenyl)piperidine-2,6-dione. ¹H NMR (500 MHz, DMSO) δ 11.60 (s, 1H), 10.95 (s, 1H), 9.86 (s, 1H), 8.45 (s, 1H), 8.23 (s, 1H), 8.04 (s, 1H), 7.85 (d, J=9.0 Hz, 1H), 7.27 (s, 1H), 7.05 (s, 1H), 7.02 (s, 1H), 6.73 (s, 1H), 4.19 (dd, J=12.6, 4.6 Hz, 1H), 3.76 (s, 3H), 3.47-3.39 (m, 3H), 2.93-2.73 (m, 8H), 2.61 (t, J=10.2 Hz, 2H), 2.54 (s, 2H), 2.24-1.99 (m, 11H), 1.84 (t, J=11.2 Hz, 2H), 1.75 (t, J=12.7 Hz, 4H), 1.62 (dd, J=23.2, 11.3 Hz, 2H), 1.42-1.22 (m, 6H), 0.74 (s, 3H). [M+H]⁺=999.7

### Example 14: (R)-3-(4-(1-(1-(1-(4-((5-bromo-4-((5-(dimethylphosphoryl)-2-ethylquinolin-6-yl)amino)pyrimidin-2-yl)amino)-2-ethyl-5-methoxyphenyl)piperidin-4-yl)azetidin-3-yl)piperidin-4-yl)-2,6-difluorophenyl)piperidine-2,6-dione

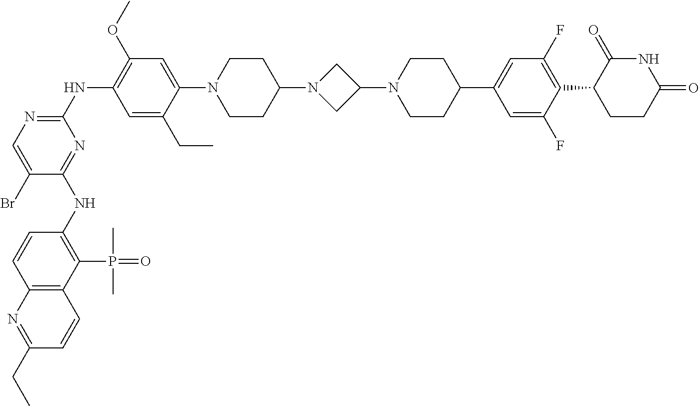 [SMILES: CCc1ccc2c(P(C)(C)=O)c(Nc3nc(Nc4cc(CC)c(N5CCC(N6CC(N7CCC(c8cc(F)c([C@H]9CCC(=O)NC9=O)c(F)c8)CC7)C6)CC5)cc4OC)ncc3Br)ccc2n1]

The titled compound (17 mg, 28%) was prepared in a manner similar to that in Example 9 step 9 from 1-(4-((5-bromo-4-((5-(dimethylphosphoryl)-2-ethylquinolin-6-yl)amino)pyrimidin-2-yl)amino)-2-ethyl-5-methoxyphenyl)piperidin-4-one (the intermediate was obtained through the similar way of Example 12 step 2) and (R)-3-(4-(1-(azetidin-3-yl)piperidin-4-yl)-2,6-difluorophenyl)piperidine-2,6-dione.

¹H NMR (500 MHz, DMSO) δ 11.78 (s, 1H), 10.95 (s, 1H), 8.56 (d, J=8.8 Hz, 1H), 8.27 (s, 1H), 8.21 (s, 1H), 7.99 (s, 1H), 7.87 (d, J=9.3 Hz, 1H), 7.44 (d, J=8.9 Hz, 1H), 7.32 (s, 1H), 7.05 (s, 1H), 7.02 (s, 1H), 6.73 (s, 1H), 4.19 (dd, J=12.4, 5.0 Hz, 1H), 3.76 (s, 3H), 3.41 (s, 2H), 2.97-2.72 (m, 10H), 2.62 (t, J=9.3 Hz, 2H), 2.54 (s, 2H), 2.26 (d, J=7.5 Hz, 2H), 2.18-2.06 (m, 2H), 2.00 (s, 1H), 2.00 (s, 3H), 1.97 (s, 3H), 1.84 (t, J=11.1 Hz, 2H), 1.75 (s, 4H), 1.67-1.57 (m, 2H), 1.32 (t, J=7.6 Hz, 5H), 0.76 (s, 3H). [M+H]⁺=998.7

### Example 15: (R)-3-(4-(1-(1-(1-(4-((5-chloro-4-((5-(dimethylphosphoryl)-3-fluoro-2-methylquinolin-6-yl)amino)pyrimidin-2-yl)amino)-5-ethoxy-2-ethylphenyl)piperidin-4-yl)azetidin-3-yl)piperidin-4-yl)-2,6-difluorophenyl)piperidine-2,6-dione

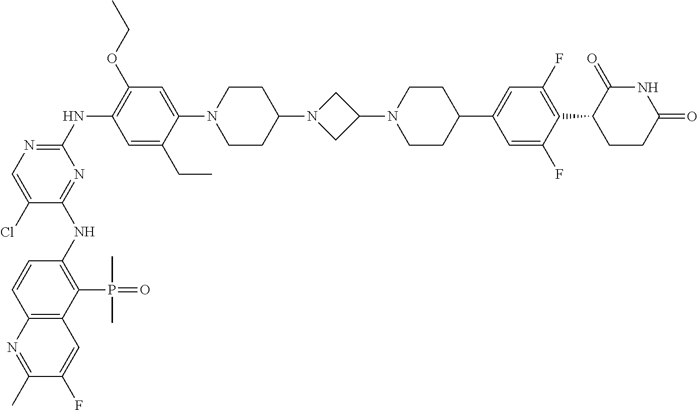 [SMILES: CCOc1cc(N2CCC(N3CC(N4CCC(c5cc(F)c([C@H]6CCC(=O)NC6=O)c(F)c5)CC4)C3)CC2)c(CC)cc1Nc1ncc(Cl)c(Nc2ccc3nc(C)c(F)cc3c2P(C)(C)=O)n1]

The titled compound (22 mg, 31%) was prepared in a manner similar to that in Example 9 step 9 from 1-(4-((5-chloro-4-((5-(dimethylphosphoryl)-3-fluoro-2-methylquinolin-6-yl)amino)pyrimidin-2-yl)amino)-5-ethoxy-2-ethylphenyl)piperidin-4-one (the intermediate was obtained through the similar way of Example 12 step 2) and (R)-3-(4-(1-(azetidin-3-yl)piperidin-4-yl)-2,6-difluorophenyl)piperidine-2,6-dione. ¹H NMR (500 MHz, DMSO) δ 11.55 (s, 1H), 10.95 (s, 1H), 8.61 (d, J=12.1 Hz, 1H), 8.32 (s, 1H), 8.16 (s, 1H), 7.95 (d, J=9.2 Hz, 1H), 7.89 (s, 1H), 7.38 (s, 1H), 7.04 (s, 1H), 7.02 (s, 1H), 6.69 (s, 1H), 4.19 (dd, J=12.6, 4.9 Hz, 1H), 4.01 (q, J=6.9 Hz, 2H), 3.41 (s, 2H), 2.92-2.72 (m, 8H), 2.64 (d, J=2.1 Hz, 3H), 2.61-2.52 (m, 4H), 2.26-2.06 (m, 4H), 2.00 (d, J=5.6 Hz, 1H), 1.98 (s, 3H), 1.95 (s, 3H), 1.84 (t, J=11.21 Hz, 2H), 1.74 (t, J=14.7 Hz, 4H), 1.61 (dd, J=22.4, 11.6 Hz, 2H), 1.34-1.23 (m, 5H), 0.71 (s, 3H). [M+H]⁺=972.7

### Example 16: (R)-3-(4-(4-(1-(1-(4-((5-bromo-4-((5-(dimethylphosphoryl)-3-fluoro-2-methylquinolin-6-yl)amino)pyrimidin-2-yl)amino)-2-ethyl-5-methoxyphenyl)piperidin-4-yl)azetidin-3-yl)piperazin-1-yl)-2,6-difluorophenyl)piperidine-2,6-dione

### Step1: tert-butyl 3-(4-(4-(2,6-dioxopiperidin-3-yl)-3,5-difluorophenyl)piperazin-1-yl)azetidine-1-carboxylate

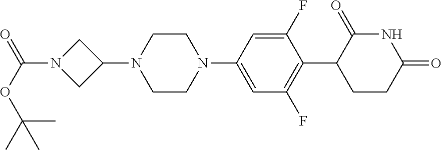 [SMILES: CC(C)(C)OC(=O)N1CC(N2CCN(c3cc(F)c(C4CCC(=O)NC4=O)c(F)c3)CC2)C1]

To a solution of 3-(2,6-difluoro-4-(piperazin-1-yl)phenyl)piperidine-2,6-dione (100 mg, 0.324 mmol) and tert-butyl 3-oxoazetidine-1-carboxylate (221.36 mg, 1.29 mmol) in DCM (3 mL) was added AcOH (77 mg, 1.29 mmol) at 20° C. The mixture was stirred at 70° C. for 1 hr. The mixture was diluted with water (20 mL), and the layers were separated. The aqueous layer was extracted with DCM (20 mL×3). The combined organic layers were washed with brine (20 mL×3), dried over Na₂SO₄, filtered and evaporated under reduced pressure to afford the crude residue, which was purified by silica gel column chromatography (DCM:MeOH)=100:0˜10:1 gradient elution) to give the desired product (100 mg, 84.8%). [M+H]⁺=465.5

### Step2: 3-(4-(4-(azetidin-3-yl)piperazin-1-yl)-2,6-difluorophenyl)piperidine-2,6-dione

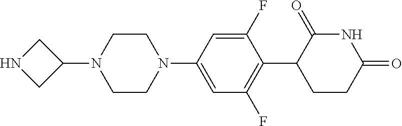 [SMILES: O=C1CCC(c2c(F)cc(N3CCN(C4CNC4)CC3)cc2F)C(=O)N1]

A solution of tert-butyl 3-(4-(4-(2,6-dioxopiperidin-3-yl)-3,5-difluorophenyl)piperazin-1-yl)azetidine-1-carboxylate (100 mg, 0.215 mmol) in 4N HCl in 1,4-dioxane (2 mL) was stirred in a round bottom flask at room temperature overnight. The mixture was concentrated under reduced pressure to afford the crude product (70 mg, 89.2%), which was used for next step without further purification. [M+H]⁺=365.5

### Step 3: (R)-3-(4-(4-(1-(1-(4-((5-bromo-4-((5-(dimethylphosphoryl)-3-fluoro-2-methylquinolin-6-yl)amino)pyrimidin-2-yl)amino)-2-ethyl-5-methoxyphenyl)piperidin-4-yl)azetidin-3-yl)piperazin-1-yl)-2,6-difluorophenyl)piperidine-2,6-dione

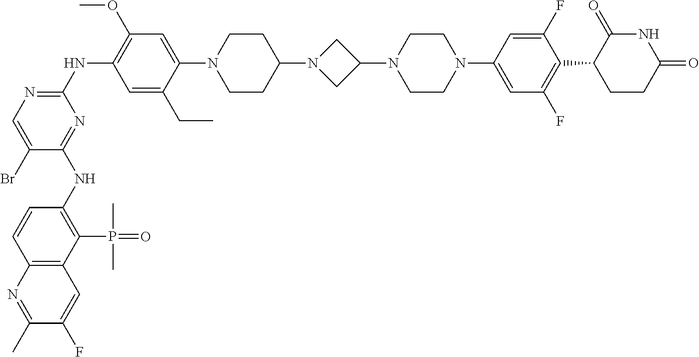 [SMILES: CCc1cc(Nc2ncc(Br)c(Nc3ccc4nc(C)c(F)cc4c3P(C)(C)=O)n2)c(OC)cc1N1CCC(N2CC(N3CCN(c4cc(F)c([C@H]5CCC(=O)NC5=O)c(F)c4)CC3)C2)CC1]

To a solution of 1-(4-((5-bromo-4-((5-(dimethylphosphoryl)-3-fluoro-2-methylquinolin-6-yl)amino)pyrimidin-2-yl)amino)-2-ethyl-5-methoxyphenyl)piperidin-4-one (the intermediate was obtained through the similar way of Example 12 step 2) (125.8 mg, 0.19 mmol) and 3-(4-(4-(azetidin-3-yl)piperazin-1-yl)-2,6-difluorophenyl)piperidine-2,6-dione (70 mg, 0.19 mmol) in DCE (10 mL) was added NaBH(OAc)₃ (120.8 mg, 0.57 mmol) at 20° C. The resulting mixture was stirred at room temperature for 1 hour. The reaction was quenched with water (10 mL) and the layers were separated. The aqueous layer was extracted with DCM (3×10 mL). The combined organic layers were dried over anhydrous Na₂SO₄, filtered and evaporated in vacuum to afford the crude residue, which was purified by silica gel column chromatography (DCM:MeOH=100:0˜10:1 gradient elution) to give an impure product, which was separated by chiral-HPLC (CHIRALPAK IF-3 4.6*50 mm, 3 μm), MtBE (0.1% DEA):(MeOH:DCM=1:1)=70:30, 1 ml/min) and the title compound corresponded to peak A @ 1.751 min/254 nm) (32 mg, 16.8%). ¹H NMR (500 MHz, DMSO) δ 11.29 (s, 1H), 10.87 (s, 1H), 8.63 (d, J=12.1 Hz, 1H), 8.22 (s, 2H), 7.94 (d, J=9.9 Hz, 2H), 7.32 (s, 1H), 6.70 (s, 1H), 6.64 (d, J=12.8 Hz, 2H), 4.06 (d, J=7.7 Hz, 1H), 3.75 (s, 3H), 3.40 (s, 8H), 3.20 (s, 4H), 2.81-2.84 (m, 6H), 2.64 (s, 5H), 2.09 (d, J=13.7 Hz, 2H), 1.95 (d, J=13.3 Hz, 8H), 1.72 (s, 2H), 1.32-1.25 (m, 2H), 0.71 (s, 3H). [M+H]⁺=1003.5.

### Example 17: (3R)-3-(4-(7-(1-(4-((5-bromo-4-((5-(dimethylphosphoryl)-3-fluoro-2-methylquinolin-6-yl)amino)pyrimidin-2-yl)amino)-2-ethyl-5-methoxyphenyl)piperidin-4-yl)-2,7-diazaspiro[4.4]nonan-2-yl)-2,6-difluorophenyl)piperidine-2,6-dione

### Step 1: tert-butyl 7-(4-(2,6-bis(benzyloxy)pyridin-3-yl)-3,5-difluorophenyl)-2,7-diazaspiro[4.4]nonane-2-carboxylate

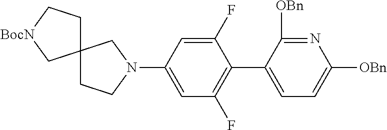 [SMILES: CC(C)(C)OC(=O)N1CCC2(CCN(c3cc(F)c(-c4ccc(OCc5ccccc5)nc4OCc4ccccc4)c(F)c3)C2)C1]

A mixture of 2,6-bis(benzyloxy)-3-(4-bromo-2,6-difluorophenyl)pyridine (960 mg, 2.0 mmol), tert-butyl 2,7-diazaspiro[4.4]nonane-2-carboxylate (680 mg, 3.0 mmol), Cs₂CO₃ (1.3 g, 4.0 mmol), Pd₂(dba)₃ (180 mg, 0.2 mmol) and RuPhos (0.10 g, 0.22 mmol) in 1,4-dioxane (40 mL) was stirred for 2 h at 100° C. under nitrogen atmosphere. The mixture was allowed to cool down to room temperature. The resulting mixture was extracted with EtOAc. The combined organic layers were washed with brine, dried over anhydrous Na₂SO₄. After filtration, the filtrate was concentrated under reduced pressure. The residue was purified by silica gel column chromatography, eluted with PE/EA (2:1) to afford tert-butyl 7-(4-(2,6-bis(benzyloxy)pyridin-3-yl)-3,5-difluorophenyl)-2,7-diazaspiro[4.4]nonane-2-carboxylate (700 mg, 55.8%). [M+H]⁺=628.2.

### Step 2: tert-butyl 7-(4-(2,6-dioxopiperidin-3-yl)-3,5-difluorophenyl)-2,7-diazaspiro[4.4]nonane-2-carboxylate

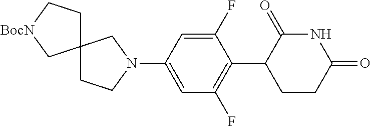 [SMILES: CC(C)(C)OC(=O)N1CCC2(CCN(c3cc(F)c(C4CCC(=O)NC4=O)c(F)c3)C2)C1]

A mixture of tert-butyl 7-(4-(2,6-bis(benzyloxy)pyridin-3-yl)-3,5-difluorophenyl)-2,7-diazaspiro[4.4]nonane-2-carboxylate (700 mg, 1.12 mmol) and Pd/C (10 wt %, 600 mg) in THF (10 mL) was stirred for 5 h at 50° C. under hydrogen atmosphere. The mixture was allowed to cool down to room temperature. The resulting mixture was filtered, the filter cake was washed with THF. The filtrate was concentrated under reduced pressure. The resulting solid was dried under vacuum. This resulted in tert-butyl 7-(4-(2,6-dioxopiperidin-3-yl)-3,5-difluorophenyl)-2,7-diazaspiro[4.4]nonane-2-carboxylate (400 mg, 81.2%). [M+H]⁺=450.3.

### Step 3: (3R)-3-(2,6-difluoro-4-(2,7-diazaspiro[4.4]nonan-2-yl)phenyl)piperidine-2,6-dione

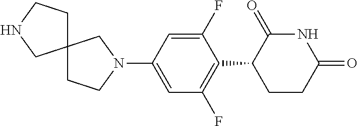 [SMILES: O=C1CC[C@H](c2c(F)cc(N3CCC4(CCNC4)C3)cc2F)C(=O)N1]

A mixture of tert-butyl 7-(4-(2,6-dioxopiperidin-3-yl)-3,5-difluorophenyl)-2,7-diazaspiro[4.4]nonane-2-carboxylate (400 mg, 0.91 mmol) in HCl (10 mL, 4N) was stirred in a flask at RT for 2 hrs. The mixture was concentrated in vacuum to afford the crude product which was purified by SFC (IH (3*25 cm, 5 um), 13% EtOH/87% CO₂, 100 bar, 100 ml/min) and the title compound corresponded to peak A @ 0.655 min/254 nm (50 mg, 34%). [M+H]⁺=350.2.

### Step 4: (3R)-3-(4-(7-(1-(4-((5-bromo-4-((5-(dimethylphosphoryl)-3-fluoro-2-methylquinolin-6-yl)amino)pyrimidin-2-yl)amino)-2-ethyl-5-methoxyphenyl)piperidin-4-yl)-2,7-diazaspiro[4.4]nonan-2-yl)-2,6-difluorophenyl)piperidine-2,6-dione

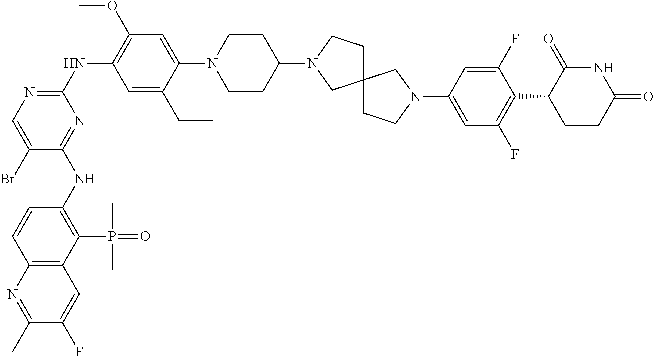 [SMILES: CCc1cc(Nc2ncc(Br)c(Nc3ccc4nc(C)c(F)cc4c3P(C)(C)=O)n2)c(OC)cc1N1CCC(N2CCC3(CCN(c4cc(F)c([C@H]5CCC(=O)NC5=O)c(F)c4)C3)C2)CC1]

The titled compound (22 mg, 24.3%) was prepared in a manner similar to that in Example 9 step 9 from 1-(4-((5-bromo-4-((5-(dimethylphosphoryl)-3-fluoro-2-methylquinolin-6-yl)amino)pyrimidin-2-yl)amino)-2-ethyl-5-methoxyphenyl)piperidin-4-one and (3R)-3-(2,6-difluoro-4-(2,7-diazaspiro[4.4]nonan-2-yl)phenyl)piperidine-2,6-dione.

¹H NMR (500 MHz, DMSO) δ 11.29 (s, 1H), 10.84 (s, 1H), 8.63 (d, J=11.7 Hz, 1H), 8.22 (s, 1H), 8.18 (s, 1H), 7.96 (s, 1H), 7.94 (s, 1H), 7.32 (s, 1H), 6.71 (s, 1H), 6.20 (s, 1H), 6.17 (s, 1H), 4.01 (dd, J=12.8, 4.7 Hz, 1H), 3.75 (s, 3H), 3.31-3.13 (m, 6H), 2.92-2.86 (m, 2H), 2.82-2.72 (m, 2H), 2.67-2.58 (m, 8H), 2.19 (ddd, J=12.3, 9.3, 3.6 Hz, 2H), 2.14-2.03 (m, 2H), 2.01-1.97 (m, 1H), 1.96 (s, 3H), 1.94 (s, 3H), 1.93-1.86 (m, 3H), 1.77 (s, 2H), 1.52 (s, 2H), 0.71 (s, 3H). [M+H]⁺=988.7

### Example 18: (R)-3-(4-(2-(1-(4-((5-bromo-4-((5-(dimethylphosphoryl)-3-fluoro-2-methylquinolin-6-yl)amino)pyrimidin-2-yl)amino)-2-ethyl-5-methoxyphenyl)piperidin-4-yl)-2,7-diazaspiro[3.5]nonan-7-yl)-2,6-difluorophenyl)piperidine-2,6-dione

### Step 1: tert-butyl 7-(4-(2,6-bis(benzyloxy)pyridin-3-yl)-3,5-difluorophenyl)-2,7-diazaspiro[3.5]nonane-2-carboxylate

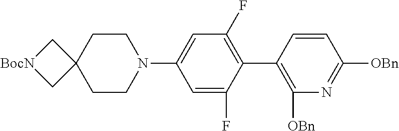 [SMILES: CC(C)(C)OC(=O)N1CC2(CCN(c3cc(F)c(-c4ccc(OCc5ccccc5)nc4OCc4ccccc4)c(F)c3)CC2)C1]

To a stirred mixture of 2,6-bis(benzyloxy)-3-(4-bromo-2,6-difluorophenyl)pyridine (1 g, 2 mmol), tert-butyl 2,7-diazaspiro[3.5]nonane-2-carboxylate (0.47 g, 2 mmol), Pd₂(dba)₃ (0.19 g, 0.2 mmol), BINAP (0.26 g, 0.4 mmol) and Cs₂CO₃ (1.4 g, 4 mmol) in dioxane (50 mL) was stirred for 16 h at 100° C. under nitrogen atmosphere. The reaction mixture was filtered and concentrated under reduced pressure. The residue was purified by silica gel column chromatography (EA:PE=0-500%) to afford the product (1.1 g, 84% yield). [M+H]⁺=628.7.

### Step 2: tert-butyl 7-(4-(2,6-dioxopiperidin-3-yl)-3,5-difluorophenyl)-2,7-diazaspiro[3.5]nonane-2-carboxylate

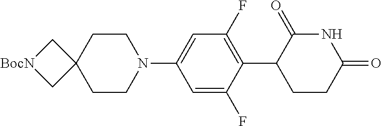 [SMILES: CC(C)(C)OC(=O)N1CC2(CCN(c3cc(F)c(C4CCC(=O)NC4=O)c(F)c3)CC2)C1]

To the solution of tert-butyl 7-(4-(2,6-bis(benzyloxy)pyridin-3-yl)-3,5-difluorophenyl)-2,7-diazaspiro[3.5]nonane-2-carboxylate (1.1 g, 1.8 mmol) in 20 mL DMF and 20 mL iPrOH, Pd/C (1 g, 10%) was added. The mixture was stirred at 40° C. for 16 hours under H₂ atmosphere (balloon). The mixture was cooled to room temperature and filtered by celite directly. The filtrate was concentrated under reduced pressure to afford the product (760 mg, 96% yield). [M+H]⁺=450.5.

### Step 3: 3-(2,6-difluoro-4-(2,7-diazaspiro[3,5]nonan-7-yl)phenyl)piperidine-2,6-dione

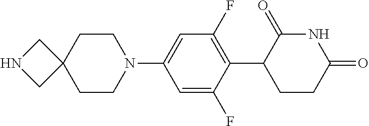 [SMILES: O=C1CCC(c2c(F)cc(N3CCC4(CC3)CNC4)cc2F)C(=O)N1]

To a stirred mixture of tert-butyl 7-(4-(2,6-dioxopiperidin-3-yl)-3,5-difluorophenyl)-2,7-diazaspiro[3.5]nonane-2-carboxylate (0.76 g, 1.7 mmol) in DCM (2 mL) were added TFA (10 mL) at room temperature. The resulting mixture was stirred for 2 h at room temperature. The reaction mixture was concentrated under reduced pressure and diluting with DCM (100 mL). The DCM was washed with NaHCO₃ (a.q. 50 mL), dried over anhydrous Na₂SO₄. After filtration, the filtrate was concentrated under reduced pressure to afford the product (0.45 g, 78% yield). [M+H]⁺=350.4.

### Step 4: (R)-3-(4-(2-(1-(4-((5-bromo-4-((5-(dimethylphosphoryl)-3-fluoro-2-methylquinolin-6-yl)amino)pyrimidin-2-yl)amino)-2-ethyl-5-methoxyphenyl)piperidin-4-yl)-2,7-diazaspiro[3.5]nonan-7-yl)-2,6-difluorophenyl)piperidine-2,6-dione

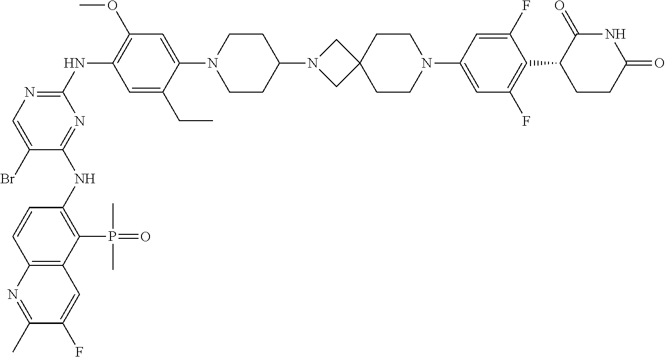 [SMILES: CCc1cc(Nc2ncc(Br)c(Nc3ccc4nc(C)c(F)cc4c3P(C)(C)=O)n2)c(OC)cc1N1CCC(N2CC3(CCN(c4cc(F)c([C@H]5CCC(=O)NC5=O)c(F)c4)CC3)C2)CC1]

A mixture of 1-(4-((5-bromo-4-((5-(dimethylphosphoryl)-3-fluoro-2-methylquinolin-6-yl)amino)pyrimidin-2-yl)amino)-2-ethyl-5-methoxyphenyl)piperidin-4-one (70 mg, 0.1 mmol), 3-(2,6-difluoro-4-(2,7-diazaspiro[3.5]nonan-7-yl)phenyl)piperidine-2,6-dione (37 mg, 0.1 mmol), STAB (45 mg, 0.2 mmol) and AOH (13 mg, 0.2 mmol) in DCE (10 mL) was stirred in a flask at 80° C. overnight. The reaction mixture was allowed to cool down to room temperature. The resulting mixture was diluted with DCM (100 mL), washed with brine (100 mL), dried over anhydrous Na₂SO₄. After filtration, the filtrate was concentrated under reduced pressure. The residue was purified by silica gel column chromatography (DCM:MEOH=0-10%) to afford the crude product which was purified by SFC (IH (3*25 cm, 5 um), 13% EtOH/87% CO₂, 100 bar, 100 ml/min) and the title compound corresponded to peak A @ 1.77 min/254 nm (4.6 mg, 4.4%). ¹H NMR (500 MHz, DMSO) δ 11.30 (s, 1H), 10.89 (s, 1H), 8.64 (d, J=12.2 Hz, 1H), 8.21 (d, J=20.2 Hz, 2H), 7.95 (d, J=9.5 Hz, 2H), 7.31 (s, 1H), 6.70 (s, 1H), 6.64 (d, J=13.2 Hz, 2H), 4.04 (dd, J=12.2, 4.0 Hz, 1H), 3.75 (s, 3H), 3.20 (s, 4H), 3.02-2.73 (m, 7H), 2.66-2.57 (m, 5H), 2.24-2.05 (m, 4H), 1.95 (d, J=13.4 Hz, 8H), 1.72 (s, 6H), 1.29 (d, J=10.3 Hz, 2H), 0.70 (s, 3H). [M+H]⁺=988.2.

### Example 19: (R)-3-(4-(7-(1-(4-((5-bromo-4-((5-(dimethylphosphoryl)-2-ethylquinazolin-6-yl)amino)pyrimidin-2-yl)amino)-5-ethoxy-2-ethylphenyl)piperidin-4-yl)-2,7-diazaspiro[3.5]nonan-2-yl)-2,6-difluorophenyl)piperidine-2,6-dione

### Step1: tert-butyl 2-(4-(2,6-bis(benzyloxy)pyridin-3-yl)-3,5-difluorophenyl)-2,7-diazaspiro[3.5]nonane-7-carboxylate

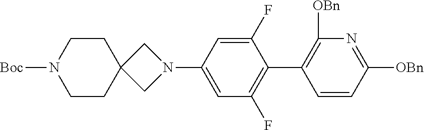 [SMILES: CC(C)(C)OC(=O)N1CCC2(CC1)CN(c1cc(F)c(-c3ccc(OCc4ccccc4)nc3OCc3ccccc3)c(F)c1)C2]

A mixture of 2,6-bis(benzyloxy)-3-(4-bromo-2,6-difluorophenyl)pyridine (200 mg, 0.415 mmol), tert-butyl 2,7-diazaspiro[3.5]nonane-7-carboxylate (103.5 mg, 0.457 mmol), Pd₂(dba)₃ (37.9 mg, 0.0415 mmol), XantPhos (48.02 g, 0.083 mmol), Cs₂CO₃ (405.6 mg, 1.245 mmol) in dioxane (5 mL) was stirred at 100° C. under nitrogen atmosphere for 15 hrs. After cooling to r.t, the reaction mixture was filtered and concentrated in vacuo. The residue was purified by silica gel column (PE:EA=1:1) to afford the product (200 mg, 76.8%). [M+H]⁺=628.3

### Step2: tert-butyl 2-(4-(2,6-dioxopiperidin-3-yl)-3,5-difluorophenyl)-2,7-diazaspiro[3.5]nonane-7-carboxylate

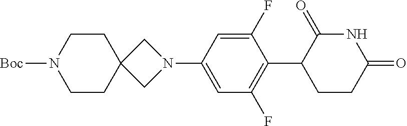 [SMILES: CC(C)(C)OC(=O)N1CCC2(CC1)CN(c1cc(F)c(C3CCC(=O)NC3=O)c(F)c1)C2]

To the solution of tert-butyl 2-(4-(2,6-bis(benzyloxy)pyridin-3-yl)-3,5-difluorophenyl)-2,7-diazaspiro[3.5]nonane-7-carboxylate (200 mg, 0.319 mmol) in 5 mL DCM and 5 mL i-PrOH, Pd/C (20 mg, 10%) was added. The mixture was stirred at 45° C. f or 16 hours under hydrogen atmosphere (balloon). Once the reaction has completed determined by LCMS, the mixture was cooled to room temperature and filtered through celite directly. The solid was dispensed in DCM (5 mL) and MeOH (50 mL), which was sonicated for 5 min. The mixture was then filtered through celite and the combined filtrate was concentrated in vacuum to afford the product (140 mg, 98.03% yield). [M+H]⁺=450.2

### Step3: 3-(2,6-difluoro-4-(2,7-diazaspiro[3.5]nonan-2-yl)phenyl)piperidine-2,6-dione

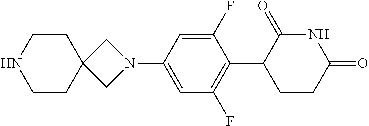 [SMILES: O=C1CCC(c2c(F)cc(N3CC4(CCNCC4)C3)cc2F)C(=O)N1]

A solution of tert-butyl 2-(4-(2,6-dioxopiperidin-3-yl)-3,5-difluorophenyl)-2,7-diazaspiro[3.5]nonane-7-carboxylate (140 mg, 0.311 mmol) in 4N HCl in 1,4-dioxane (4 mL) was stirred in a round bottom flask at room temperature overnight. The mixture was concentrated under reduced pressure to afford the crude product (100 mg, 91.9%), which was used for next step without further purification. [M+H]⁺=350.2.

### Step 4: (R)-3-(4-(7-(1-(4-((5-bromo-4-((5-(dimethylphosphoryl)-2-ethylquinazolin-6-yl)amino)pyrimidin-2-yl)amino)-5-ethoxy-2-ethylphenyl)piperidin-4-yl)-2,7-diazaspiro[3.5]nonan-2-yl)-2,6-difluorophenyl)piperidine-2,6-dione

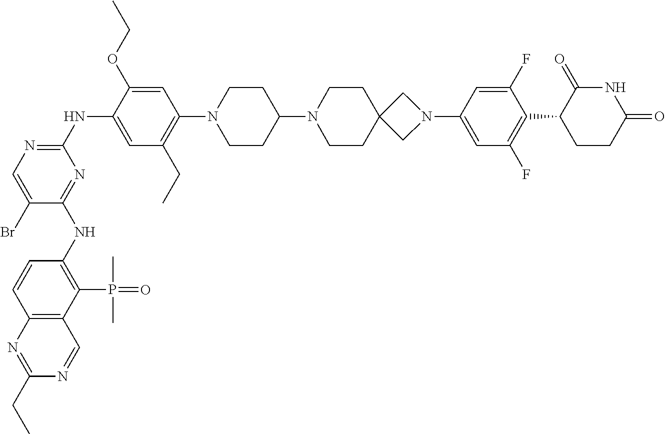 [SMILES: CCOc1cc(N2CCC(N3CCC4(CC3)CN(c3cc(F)c([C@H]5CCC(=O)NC5=O)c(F)c3)C4)CC2)c(CC)cc1Nc1ncc(Br)c(Nc2ccc3nc(CC)ncc3c2P(C)(C)=O)n1]

To a solution of 1-(4-((5-bromo-4-((5-(dimethylphosphoryl)-2-ethylquinazolin-6-yl)amino)pyrimidin-2-yl)amino)-5-ethoxy-2-ethylphenyl)piperidin-4-one (the intermediate was obtained through the similar way of Example 12 step 2) (100 mg, 0.15 mmol) and 3-(2,6-difluoro-4-(2,7-diazaspiro[3.5]nonan-2-yl)phenyl)piperidine-2,6-dione (62.9 mg, 0.18 mmol) in DCE (10 mL) was added NaBH(OAc)₃ (95.37 mg, 0.45 mmol) at 20° C. The resulting mixture was stirred at room temperature for 1 hour. The reaction was quenched with water (10 mL) and the layers were separated. The aqueous layer was extracted with DCM (3×10 mL). The combined organic layers were dried over anhydrous Na₂SO₄, filtered and evaporated in vacuum to afford the crude residue, which was purified by silica gel column chromatography (DCM:MeOH=100:0˜10:1 gradient elution) to give an impure product, which was separated by chiral-HPLC (CHIRALPAK IF-3 4.6*50 mm, 3 μm), MtBE (0.1% DEA):(MeOH:DCM=1:1)=70:30, 1 ml/min) and the title compound corresponded to peak A @ 1.723 min/254 nm) (38 mg, 25.4%). ¹H NMR (500 MHz, DMSO) δ 11.53 (s, 1H), 10.85 (s, 1H), 9.90 (s, 1H), 8.44 (s, 1H), 8.25 (s, 1H), 7.95 (s, 1H), 7.85 (d, J=9.4 Hz, 1H), 7.32 (s, 1H), 6.70 (s, 1H), 6.11 (d, J=11.8 Hz, 2H), 4.00 (s, 2H), 3.55 (s, 5H), 3.06 (d, J=7.2 Hz, 2H), 2.92 (d, J=9.6 Hz, 3H), 2.76 (d, J=11.6 Hz, 2H), 2.62 (s, 4H), 2.36 (s, 1H), 2.22 (s, 2H), 2.03 (d, J=13.2 Hz, 8H), 1.95 (s, 1H), 1.74 (s, 6H), 1.57 (d, J=10.6 Hz, 2H), 1.39 (d, J=7.4 Hz, 3H), 1.23-1.26 (m, 3H), 0.69 (s, 3H). [M+H]⁺=999.4.

### Example 20: (R)-3-(4-(2-(1-(4-((5-bromo-4-((5-(dimethylphosphoryl)-2-ethylquinazolin-6-yl)amino)pyrimidin-2-yl)amino)-2-ethyl-5-methoxyphenyl)piperidin-4-yl)-2,7-diazaspiro[3.5]nonan-7-yl)-2,6-difluorophenyl)piperidine-2,6-dione

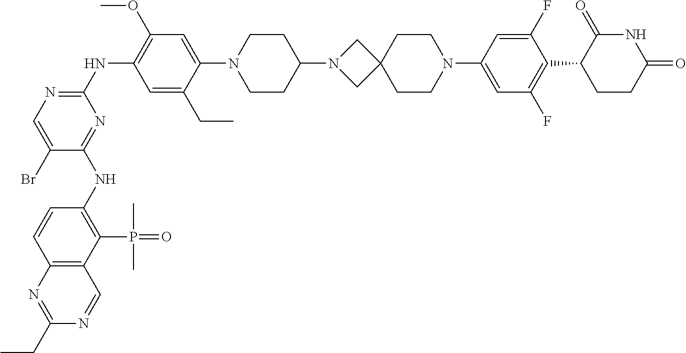 [SMILES: CCc1ncc2c(P(C)(C)=O)c(Nc3nc(Nc4cc(CC)c(N5CCC(N6CC7(CCN(c8cc(F)c([C@H]9CCC(=O)NC9=O)c(F)c8)CC7)C6)CC5)cc4OC)ncc3Br)ccc2n1]

A mixture of 1-(4-((5-bromo-4-((5-(dimethylphosphoryl)-2-ethylquinazolin-6-yl)amino)pyrimidin-2-yl)amino)-2-ethyl-5-methoxyphenyl)piperidin-4-one (70 mg, 0.11 mmol), 3-(2,6-difluoro-4-(2,7-diazaspiro[3.5]nonan-7-yl)phenyl)piperidine-2,6-dione (38 mg, 0.11 mmol), STAB (45 mg, 0.21 mmol) and AOH (13 mg, 0.21 mmol) in DCE (10 mL) was stirred in a flask at 80° C. overnight. The reaction mixture was allowed to cool down to room temperature. The resulting mixture was diluted with DCM (100 mL), washed with brine (100 mL), dried over anhydrous Na₂SO₄. After filtration, the filtrate was concentrated under reduced pressure. The residue was purified by silica gel column chromatography (DCM:MEOH=0-10%) to afford the crude product which was purified by SFC (IH (3*25 cm, 5 um), 13% EtOH/87% CO₂, 100 bar, 100 ml/min) and the title compound corresponded to peak A @ 1.29 min/254 nm (6.1 mg, 5.8%). ¹H NMR (500 MHz, DMSO) δ 11.56 (s, 1H), 10.87 (s, 1H), 9.88 (s, 1H), 8.45 (s, 1H), 8.24 (s, 1H), 8.03 (s, 1H), 7.86 (d, J=9.9 Hz, 1H), 7.28 (s, 1H), 6.73 (s, 1H), 6.65 (d, J=13.5 Hz, 2H), 4.04 (dd, J=12.9, 4.9 Hz, 1H), 3.76 (s, 3H), 3.29 (s, 1H), 3.21 (s, 4H), 3.08-3.03 (m, 2H), 2.91 (s, 4H), 2.77 (dd, J=21.4, 9.3 Hz, 1H), 2.63 (t, J=10.3 Hz, 2H), 2.51 (s, 1H), 2.22 (s, 2H), 2.08 (d, J=10.1 Hz, 1H), 2.03 (d, J=13.4 Hz, 6H), 1.96 (d, J=5.6 Hz, 2H), 1.75 (s, 5H), 1.41-1.22 (m, 7H), 0.74 (s, 3H). [M+H]⁺=985.2.

### Example 21: 3-(4-(3-(4-(1-(4-((5-bromo-4-((5-(dimethylphosphoryl)-2-ethylquinolin-6-yl)amino)pyrimidin-2-yl)amino)-2-ethyl-5-methoxyphenyl)piperidin-4-yl)piperazin-1-yl)azetidin-1-yl)-3-methyl-2-oxo-2,3-dihydro-1H-benzol[d]imidazol-1-yl)piperidine-2,6-dione

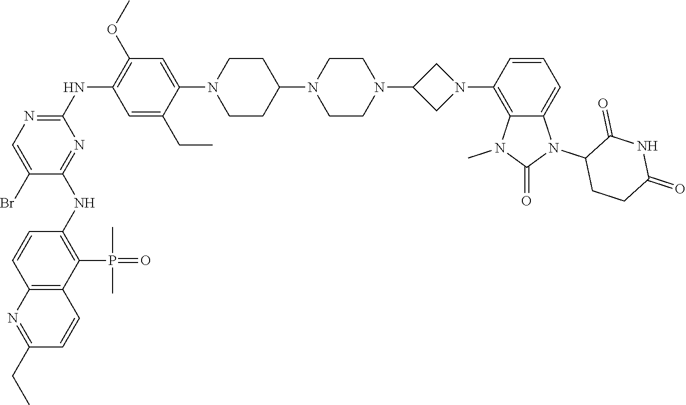 [SMILES: CCc1ccc2c(P(C)(C)=O)c(Nc3nc(Nc4cc(CC)c(N5CCC(N6CCN(C7CN(c8cccc9c8n(C)c(=O)n9C8CCC(=O)NC8=O)C7)CC6)CC5)cc4OC)ncc3Br)ccc2n1]

### Step 1: 1-(2,6-bis(benzyloxy)pyridin-3-yl)-4-(3-hydroxyazetidin-1-yl)-3-methyl-1,3-dihydro-2H-benzo[d]imidazol-2-one

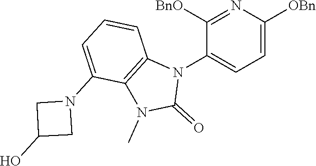 [SMILES: Cn1c(=O)n(-c2ccc(OCc3ccccc3)nc2OCc2ccccc2)c2cccc(N3CC(O)C3)c21]

A mixture of 1-(2,6-bis(benzyloxy)pyridin-3-yl)-4-bromo-3-methyl-1,3-dihydro-2H-benzo[d]imidazol-2-one (1.03 g, 2.0 mmol), azetidin-3-ol (220 mg, 3.0 mmol), Cs₂CO₃ (1.3 g, 4.0 mmol), Pd₂(dba)₃ (180 mg, 0.2 mmol) and RuPhos (0.10 g, 0.22 mmol) in 1,4-dioxane (40 mL) was stirred for 2 h at 100° C. under nitrogen atmosphere. The mixture was allowed to cool down to room temperature. The resulting mixture was extracted with EtOAc. The combined organic layers were washed with brine, dried over anhydrous Na₂SO₄. After filtration, the filtrate was concentrated under reduced pressure. The residue was purified by silica gel column chromatography, eluted with PE/EA (1:1) to afford 1-(2,6-bis(benzyloxy)pyridin-3-yl)-4-(3-hydroxyazetidin-1-yl)-3-methyl-1,3-dihydro-2H-benzo[d]imidazol-2-one (800 mg, 78.7%). [M+H]⁺=509.2.

### Step 2: 3-(4-(3-hydroxyazetidin-1-yl)-3-methyl-2-oxo-2,3-dihydro-1H-benzo[d]imidazol-1-yl)piperidine-2,6-dione

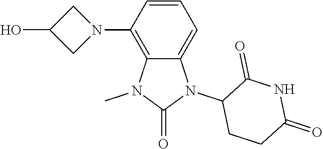 [SMILES: Cn1c(=O)n(C2CCC(=O)NC2=O)c2cccc(N3CC(O)C3)c21]

A mixture of 1-(2,6-bis(benzyloxy)pyridin-3-yl)-4-(3-hydroxyazetidin-1-yl)-3-methyl-1,3-dihydro-2H-benzo[d]imidazol-2-one (800 mg, 1.57 mmol) and Pd/C (10 wt %, 600 mg) in THF (10 mL) was stirred for 5 h at 50° C. under hydrogen atmosphere. The mixture was allowed to cool down to room temperature. The resulting mixture was filtered, the filter cake was washed with THF. The filtrate was concentrated under reduced pressure. This resulted in 3-(4-(3-hydroxyazetidin-1-yl)-3-methyl-2-oxo-2,3-dihydro-1H-benzo[d]imidazol-1-yl)piperidine-2,6-dione (380 mg, 73.1%) after drying under vacuum. [M+H]⁺=331.2.

### Step 3: 3-(3-methyl-2-oxo-4-(3-oxoazetidin-1-yl)-2,3-dihydro-1H-benzo[d]imidazol-1-yl)piperidine-2,6-dione

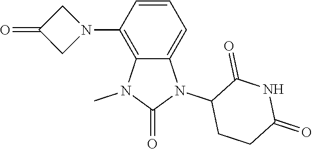 [SMILES: Cn1c(=O)n(C2CCC(=O)NC2=O)c2cccc(N3CC(=O)C3)c21]

A mixture of 3-(4-(3-hydroxyazetidin-1-yl)-3-methyl-2-oxo-2,3-dihydro-1H-benzo[d]imidazol-1-yl)piperidine-2,6-dione (380 mg, 1.15 mmol) and IBX (480 mg, 1.72 mmol) in DMSO (10 mL) was stirred in a flask at room temperature overnight. The reaction was quenched with water and the mixture was extracted with EtOAc, washed three times with saturated aqueous NaCl and twice with saturated aqueous NaHCO₃.

The organic layer was dried over anhydrous Na₂SO₄ and evaporated in vacuum to afford the product (310 mg, 81.2%). [M+H]⁺=329.2.

### Step 4: 3-(4-(3-(4-(1-(4-((5-bromo-4-((5-(dimethylphosphoryl)-2-ethylquinolin-6-yl)amino)pyrimidin-2-yl)amino)-2-ethyl-5-methoxyphenyl)piperidin-4-yl)piperazin-1-yl)azetidin-1-yl)-3-methyl-2-oxo-2,3-dihydro-1H-benzo[d]imidazol-1-yl)piperidine-2,6-dione

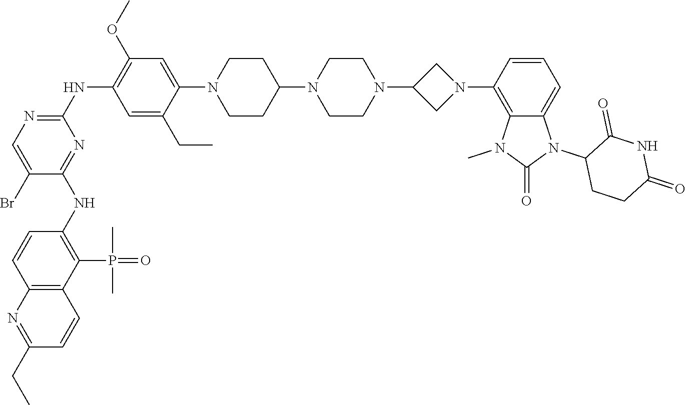 [SMILES: CCc1ccc2c(P(C)(C)=O)c(Nc3nc(Nc4cc(CC)c(N5CCC(N6CCN(C7CN(c8cccc9c8n(C)c(=O)n9C8CCC(=O)NC8=O)C7)CC6)CC5)cc4OC)ncc3Br)ccc2n1]

The titled compound (22 mg, 27%) was prepared in a manner similar to that in Example 1 step 9 from (6-((5-bromo-2-((5-ethyl-2-methoxy-4-(4-(piperazin-1-yl)piperidin-1-yl)phenyl)amino)pyrimidin-4-yl)amino)-2-ethylquinolin-5-yl)dimethylphosphine oxide and 3-(3-methyl-2-oxo-4-(3-oxoazetidin-1-yl)-2,3-dihydro-1H-benzol[d]imidazol-1-yl)piperidine-2,6-dione.

¹H NMR (500 MHz, DMSO) δ 11.80 (s, 1H), 11.08 (s, 1H), 8.55 (d, J=8.9 Hz, 1H), 8.27 (s, 1H), 8.21 (s, 1H), 8.00 (s, 1H), 7.87 (d, J=9.5 Hz, 1H), 7.44 (d, J=8.9 Hz, 1H), 7.33 (s, 1H), 6.96 (t, J=8.0 Hz, 1H), 6.75 (s, 1H), 6.74 (s, 1H), 6.68 (d, J=8.1 Hz, 1H), 5.32 (dd, J=12.6, 5.1 Hz, 1H), 3.90 (d, J=4.4 Hz, 2H), 3.76 (s, 3H), 3.64 (s, 2H), 3.57 (s, 3H), 3.29 (s, 2H), 3.24-3.16 (m, 1H), 2.96-2.84 (m, 5H), 2.73-2.53 (m, 8H), 2.43-2.26 (m, 6H), 2.00 (s, 3H), 1.97 (s, 3H), 1.84 (d, J=10.5 Hz, 2H), 1.55 (d, J=9.6 Hz, 2H), 1.32 (t, J=7.6 Hz, 3H), 0.77 (s, 3H). [M+H]⁺=1033.7

### Example 22: 3-(4-(3-(4-(1-(4-((5-bromo-4-((5-(dimethylphosphoryl)-3-fluoro-2-methylquinolin-6-yl)amino)pyrimidin-2-yl)amino)-2-ethyl-5-methoxyphenyl)piperidin-4-yl)piperazin-1-yl)azetidin-1-yl)-3-methyl-2-oxo-2,3-dihydro-1H-benzo[d]imidazol-1-yl)piperidine-2,6-dione

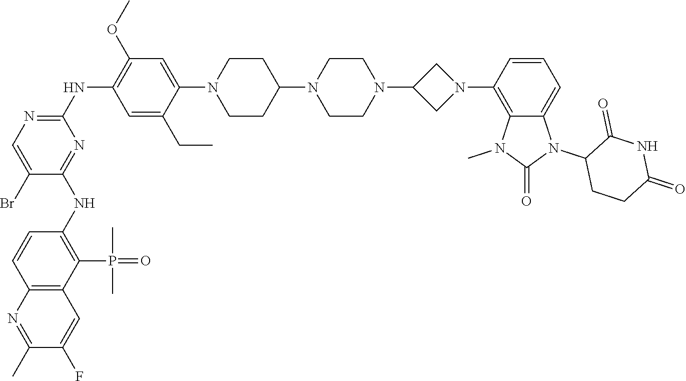 [SMILES: CCc1cc(Nc2ncc(Br)c(Nc3ccc4nc(C)c(F)cc4c3P(C)(C)=O)n2)c(OC)cc1N1CCC(N2CCN(C3CN(c4cccc5c4n(C)c(=O)n5C4CCC(=O)NC4=O)C3)CC2)CC1]

The titled compound (19 mg, 26%) was prepared in a manner similar to that in Example 1 step 9 from (6-((5-bromo-2-((5-ethyl-2-methoxy-4-(4-(piperazin-1-yl)piperidin-1-yl)phenyl)amino)pyrimidin-4-yl)amino)-3-fluoro-2-methylquinolin-5-yl)dimethylphosphine oxide (the intermediate was obtained through the similar way with Example 7 step 7) and 3-(3-methyl-2-oxo-4-(3-oxoazetidin-1-yl)-2,3-dihydro-1H-benzo[d]imidazol-1-yl)piperidine-2,6-dione.

¹H NMR (500 MHz, DMSO) δ 11.29 (s, 1H), 11.08 (s, 1H), 8.64 (d, J=9.4 Hz, 1H), 8.22 (s, 1H), 8.18 (s, 1H), 7.95 (s, 1H), 7.93 (s, 1H), 7.34 (s, 1H), 6.96 (t, J=8.0 Hz, 1H), 6.75 (d, J=7.9 Hz, 1H), 6.71 (s, 1H), 6.67 (d, J=8.2 Hz, 1H), 5.32 (dd, J=12.4, 5.1 Hz, 1H), 3.89 (d, J=4.3 Hz, 2H), 3.75 (s, 3H), 3.63 (s, 2H), 3.57 (s, 3H), 3.46-3.41 (m, 2H), 3.21-3.17 (m, 1H), 2.88 (dd, J=21.8, 8.3 Hz, 3H), 2.62 (d, J=18.8 Hz, 9H), 2.39-2.25 (m, 5H), 2.18 (s, 2H), 1.99 (d, J=5.6 Hz, 1H), 1.96 (s, 3H), 1.94 (s, 3H), 1.82 (d, J=10.5 Hz, 2H), 1.53 (d, J=10.0 Hz, 2H), 0.70 (s, 3H). [M+H]⁺=1037.7

### Example 23: 3-(4-(3-(4-(1-(4-((5-bromo-4-((5-(dimethylphosphoryl)-2-ethylquinazolin-6-yl)amino)pyrimidin-2-yl)amino)-2-ethyl-5-methoxyphenyl)piperidin-4-yl)piperazin-1-yl)azetidin-1-yl)-3-methyl-2-oxo-2,3-dihydro-1H-benzo[d]imidazol-1-yl)piperidine-2,6-dione

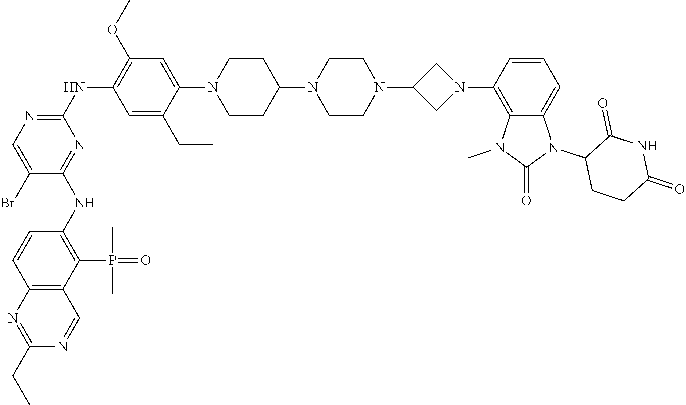 [SMILES: CCc1ncc2c(P(C)(C)=O)c(Nc3nc(Nc4cc(CC)c(N5CCC(N6CCN(C7CN(c8cccc9c8n(C)c(=O)n9C8CCC(=O)NC8=O)C7)CC6)CC5)cc4OC)ncc3Br)ccc2n1]

The titled compound (21 mg, 27%) was prepared in a manner similar to that in Example 1 step 9 from (6-((5-bromo-2-((5-ethyl-2-methoxy-4-(4-(piperazin-1-yl)piperidin-1-yl)phenyl)amino)pyrimidin-4-yl)amino)-2-ethylquinazolin-5-yl)dimethylphosphine oxide and 3-(3-methyl-2-oxo-4-(3-oxoazetidin-1-yl)-2,3-dihydro-1H-benzo[d]imidazol-1-yl)piperidine-2,6-dione.

¹H NMR (500 MHz, DMSO) δ 11.61 (s, 1H), 11.08 (s, 1H), 9.85 (s, 1H), 8.45 (s, 1H), 8.23 (s, 1H), 8.05 (s, 1H), 7.85 (d, J=9.0 Hz, 1H), 7.27 (s, 1H), 6.96 (t, J=8.0 Hz, 1H), 6.74 (s, 2H), 6.68 (d, J=8.0 Hz, 1H), 5.32 (dd, J=12.6, 5.3 Hz, 1H), 3.90 (d, J=4.2 Hz, 2H), 3.75 (s, 3H), 3.64 (s, 2H), 3.57 (s, 3H), 3.23-3.17 (m, 1H), 3.05 (q, J=7.6 Hz, 2H), 2.93 (d, J=10.3 Hz, 2H), 2.90-2.82 (m, 1H), 2.74-2.53 (m, 9H), 2.43-2.24 (m, 6H), 2.04 (s, 3H), 2.02 (s, 3H), 2.01-1.95 (m, 1H), 1.84 (d, J=11.7 Hz, 2H), 1.54 (d, J=9.5 Hz, 2H), 1.37 (t, J=7.6 Hz, 3H), 0.76 (s, 3H). [M+H]⁺=1034.7

### Example 24: (S)-3-(4-(3-(4-(1-(4-((5-bromo-4-((5-(dimethylphosphoryl)-3-fluoro-2-methylquinolin-6-yl)amino)pyrimidin-2-yl)amino)-2-ethyl-5-methoxyphenyl)piperidin-4-yl)piperazine-1-carbonyl)azetidin-1-yl)-7-fluoro-3-methyl-2-oxo-2,3-dihydro-1H-benzo[d]imidazol-1-yl)piperidine-2,6-dione

### Step 1: 2-((2,6-bis(benzyloxy)pyridin-3-yl)amino)-6-bromo-3-fluorobenzoic acid

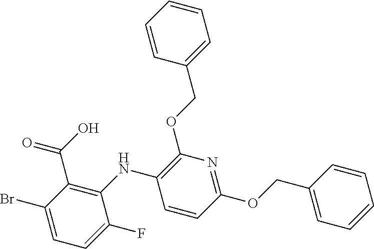 [SMILES: O=C(O)c1c(Br)ccc(F)c1Nc1ccc(OCc2ccccc2)nc1OCc1ccccc1]

To a solution of 2,6-bis(benzyloxy)pyridin-3-amine (15 g, 49.0 mmol) in THF (300 mL) was added LiHMDS (1 M in THF, 80 mL) dropwise in 30 min at 0° C., the reaction solution was stirred for 60 min at this temperature, then to this was added 6-bromo-2,3-difluorobenzoic acid (10 g, 42.4 mmol) in THF (50 mL) dropwise in 20 min. The resulting solution was stirred for 12 h at 10-20° C. The reaction was quenched by the addition of sat. aq. NH₄Cl solution. Concentrated. The residue was purified by a silica gel column, eluted with DCM/MeOH (5:1) to afford the product (20 g, 90.4%). [M+H]⁺=523.2.

### Step 2: 1-(2,6-bis(benzyloxy)pyridin-3-yl)-4-bromo-7-fluoro-1,3-dihydro-2H-benzo[d]imidazol-2-one

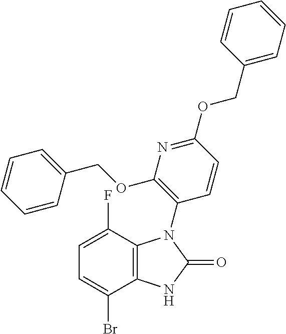 [SMILES: O=c1[nH]c2c(Br)ccc(F)c2n1-c1ccc(OCc2ccccc2)nc1OCc1ccccc1]

To a solution of 2-((2,6-bis(benzyloxy)pyridin-3-yl) amino)-6-bromo-3-fluorobenzoic acid (20 g, 38.3 mmol) in DMA (400 mL) were added TEA (11.6 g, 114.8 mmol) and DPPA (15.8 g, 57.4 mmol). The mixture was stirred for 12 h at 80° C. under nitrogen atmosphere. The mixture was quenched by water at 30° C. and was extracted with EtOAc. The combined organic phases were concentrated under reduced pressure.

The residue was purified by a silica gel column, eluted with DCM/EtOAc (1:1) to afford the product (18 g, 90.9%). [M+H]⁺=520.1.

### Step 3: 1-(2,6-bis(benzyloxy)pyridin-3-yl)-4-bromo-7-fluoro-3-methyl-1,3-dihydro-2H-benzo[d]imidazol-2-one

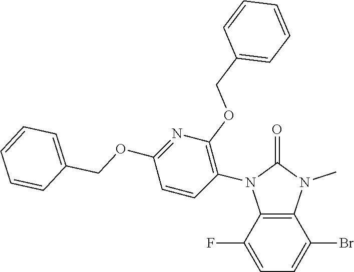 [SMILES: Cn1c(=O)n(-c2ccc(OCc3ccccc3)nc2OCc2ccccc2)c2c(F)ccc(Br)c21]

To a solution of 1-(2,6-bis(benzyloxy)pyridin-3-yl)-4-bromo-7-fluoro-1,3-dihydro-2H-benzo[d]imidazol-2-one (10 g, 19.2 mmol) in DMF (100 mL) was added Cs₂CO₃ (18.7 g, 57.5 mmol) at 0° C. for 10 min. Then to this was added CH₃I (8.2 g, 57.7 mmol). The resulting solution was stirred for 12 h at 10-20° C. The mixture was quenched by water at 30° C. and was extracted with EtOAc. The combined organic phases were concentrated under reduced pressure. The residue was purified by a silica gel column, eluted with DCM/EtOAc (1:1) to afford the product (8 g, 77.9%). [M+H]⁺=534.3.

### Step 4: benzyl 1-(1-(2,6-bis(benzyloxy)pyridin-3-yl)-7-fluoro-3-methyl-2-oxo-2,3-dihydro-1H-benzo[d]imidazol-4-yl)azetidine-3-carboxylate

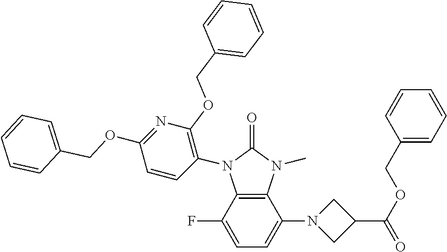 [SMILES: Cn1c(=O)n(-c2ccc(OCc3ccccc3)nc2OCc2ccccc2)c2c(F)ccc(N3CC(C(=O)OCc4ccccc4)C3)c21]

To a solution of 1-(2,6-bis(benzyloxy)pyridin-3-yl)-4-bromo-7-fluoro-3-methyl-1,3-dihydro-2H-benzo[d]imidazol-2-one (4 g, 7.4 mmol) in dioxane (40 mL) were added benzyl azetidine-3-carboxylate TFA salt (3.0 g, 10.4 mmol), Ruphos (0.6 g, 1.2 mmol), Pd₂(dba)₃ (0.6 g, 0.6 mmol) and Cs₂CO₃ (9.6 g, 29.6 mmol). The resulting solution was stirred for 3 h at 90° C. under nitrogen atmosphere. The resulting mixture was concentrated under reduced pressure. The residue was purified by a silica gel column, eluted with DCM/EtOAc (1:1) to afford the product (2.36 g, 49.5%). [M+H]⁺=645.2.

### Step 5: 1-(1-(2,6-dioxopiperidin-3-yl)-7-fluoro-3-methyl-2-oxo-2,3-dihydro-1H-benzo[d]imidazol-4-yl)azetidine-3-carboxylic acid

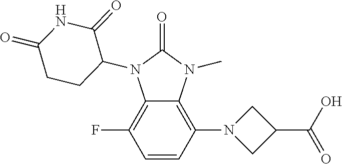 [SMILES: Cn1c(=O)n(C2CCC(=O)NC2=O)c2c(F)ccc(N3CC(C(=O)O)C3)c21]

To a solution of benzyl 1-(1-(2,6-bis(benzyloxy)pyridin-3-yl)-7-fluoro-3-methyl-2-oxo-2,3-dihydro-1H-benzo[d]imidazol-4-yl)azetidine-3-carboxylate (1.4 g, 2.2 mmol) in THF (30 mL) were added Pd/C (1.4 g, 10% wt) and CH₃COOH (0.3 mL). The resulting solution was stirred for 8 h at 30° C. under H₂ atmosphere.

After filtration, the filtrate was concentrated under reduced pressure. This was resulted in 1-(1-(2,6-dioxopiperidin-3-yl)-7-fluoro-3-methyl-2-oxo-2,3-dihydro-1H-benzo[d]imidazol-4-yl)azetidine-3-carboxylic acid (0.5 g, 65.6%). H NMR (500 MHz, DMSO) δ 12.48 (s, 1H), 11.10 (d, J=8.4 Hz, 1H), 7.30-7.05 (m, 1H), 6.98-6.79 (m, 1H), 5.49 (s, 1H), 4.08-3.91 (m, 1H), 3.85 (td, J=6.6, 2.7 Hz, 1H), 3.53 (s, 2H), 3.50-3.35 (m, 3H), 2.97 (d, J=14.8 Hz, 1H), 2.61 (d, J=18.1 Hz, 1H), 2.38-2.02 (m, 2H), 1.95 (d, J=23.9 Hz, 1H). [M+H]⁺=377.1.

### Step 6: (S)-3-(4-(3-(4-(1-(4-((5-bromo-4-((5-(dimethylphosphoryl)-3-fluoro-2-methylquinolin-6-yl)amino)pyrimidin-2-yl)amino)-2-ethyl-5-methoxyphenyl)piperidin-4-yl)piperazine-1-carbonyl)azetidin-1-yl)-7-fluoro-3-methyl-2-oxo-2,3-dihydro-1H-benzo[d]imidazol-1-yl)piperidine-2,6-dione

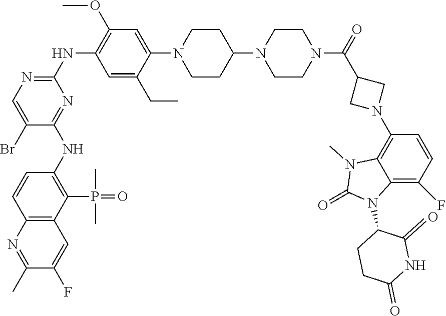

To a solution of (6-((5-bromo-2-((5-ethyl-2-methoxy-4-(4-(piperazin-1-yl)piperidin-1-yl)phenyl)amino)pyrimidin-4-yl)amino)-3-fluoro-2-methylquinolin-5-yl)dimethylphosphine oxide (80 mg, 0.11 mmol), 1-(1-(2,6-dioxopiperidin-3-yl)-7-fluoro-3-methyl-2-oxo-2,3-dihydro-1H-benzo[d]imidazol-4-yl)azetidine-3-carboxylic acid (41.4 mg, 0.11 mmol) and DIEA (42.63 mg, 0.33 mmol) in DCM (3 mL) was added T3P (139.99 mg, 0.44 mmol) dropwise at room temperature. The resulting mixture was stirred at room temperature for 1 hour. The reaction was quenched with water (10 mL) and extracted with DCM (2×10 mL). The combined organic layers were dried over anhydrous Na₂SO₄, filtered and evaporated in vacuum to afford the crude residue, which was purified by silica gel column chromatography (DCM:MeOH=100:0˜10:1 gradient elution) to give an impure product, which was separated by chiral-HPLC (CHIRALPAK IF-3 4.6*50 mm, 3 μm), MtBE (0.1% DEA):(MeOH:DCM=1:1)=50:50, 1 ml/min) and the title compound corresponded to peak A @ 1.719 min/254 nm) (20 mg, 16.8%).

¹H NMR (500 MHz, DMSO) δ 11.30 (s, 1H), 11.12 (s, 1H), 8.64 (d, J=11.4 Hz, 1H), 8.23 (s, 2H), 7.96 (s, 2H), 7.33 (s, 1H), 6.88 (t, 1H), 6.79 (d, J=4.8 Hz, 1H), 6.72 (s, 1H), 5.54-5.45 (m, 1H), 3.94 (d, J=34.1 Hz, 4H), 3.75 (s, 4H), 3.68 (s, 3H), 3.60 (s, 1H), 3.49 (s, 2H), 3.05-2.86 (m, 3H), 2.64 (s, 6H), 2.52 (d, J=18.1 Hz, 5H), 2.36 (s, 1H), 2.18 (s, 3H), 2.09 (s, 1H), 1.95 (d, J=13.4 Hz, 6H), 1.81 (s, 2H), 1.56 (s, 2H), 0.70 (s, 3H). [M+H]⁺=1083.3.

### Example 25: 3-(4-(6-(1-(4-((5-bromo-4-((5-(dimethylphosphoryl)-2-ethylquinazolin-6-yl)amino)pyrimidin-2-yl)amino)-2-ethyl-5-methoxyphenyl)piperidin-4-yl)-2,6-diazaspiro[3.3]heptan-2-yl)-3-methyl-2-oxo-2,3-dihydro-1H-benzo[d]imidazol-1-yl)piperidine-2,6-dione

### Step 1: tert-butyl 6-(1-(2,6-bis(benzyloxy)pyridin-3-yl)-3-methyl-2-oxo-2,3-dihydro-1H-benzo[d]imidazol-4-yl)-2,6-diazaspiro[3.3]heptane-2-carboxylate

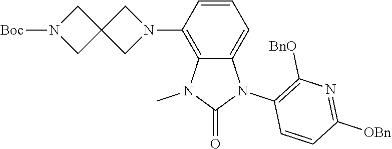 [SMILES: Cn1c(=O)n(-c2ccc(OCc3ccccc3)nc2OCc2ccccc2)c2cccc(N3CC4(CN(C(=O)OC(C)(C)C)C4)C3)c21]

To a stirred mixture of 1-(2,6-bis(benzyloxy)pyridin-3-yl)-4-bromo-3-methyl-1,3-dihydro-2H-benzo[d]imidazol-2-one (1 g, 1.9 mmol), tert-butyl 2,6-diazaspiro[3.3]heptane-2-carboxylate (0.38 g, 1.9 mmol), Pd₂(dba)₃ (0.17 g, 0.2 mmol), Xantphos (0.23 g, 0.4 mmol) and Cs₂CO₃ (1.3 g, 3.8 mmol) in dioxane (50 mL) was stirred for 16 h at 100° C. under nitrogen atmosphere. The reaction mixture was filtered and concentrated under reduced pressure. The residue was purified by silica gel column chromatography (EA:PE=0-50%) to afford the product (1.1 g, 89% yield). [M+H]⁺=634.5.

### Step 2: tert-butyl 6-(1-(2,6-dioxopiperidin-3-yl)-3-methyl-2-oxo-2,3-dihydro-1H-benzo[d]imidazol-4-yl)-2,6-diazaspiro[3.3]heptane-2-carboxylate

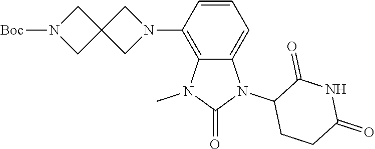 [SMILES: Cn1c(=O)n(C2CCC(=O)NC2=O)c2cccc(N3CC4(CN(C(=O)OC(C)(C)C)C4)C3)c21]

To the solution of tert-butyl 6-(1-(2,6-bis(benzyloxy)pyridin-3-yl)-3-methyl-2-oxo-2,3-dihydro-1H-benzo[d]imidazol-4-yl)-2,6-diazaspiro[3.3]heptane-2-carboxylate (1.1 g, 1.7 mmol) in 20 mL DMF and 20 mL iPrOH, Pd/C (1 g, 10%) was added. The mixture was stirred at 40° C. for 16 hours under H₂ atmosphere (balloon). The mixture was cooled to room temperature and filtered by celite directly. The filtrate was concentrated under reduced pressure to afford the product (670 mg, 82% yield). [M+H]⁺=456.3.

### Step 3: 3-(3-methyl-2-oxo-4-(2,6-diazaspiro[3.3]heptan-2-yl)-2,3-dihydro-1H-benzo[d]imidazol-1-yl)piperidine-2,6-dione

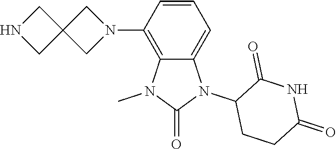 [SMILES: Cn1c(=O)n(C2CCC(=O)NC2=O)c2cccc(N3CC4(CNC4)C3)c21]

To a stirred mixture of tert-butyl 6-(1-(2,6-dioxopiperidin-3-yl)-3-methyl-2-oxo-2,3-dihydro-1H-benzo[d]imidazol-4-yl)-2,6-diazaspiro[3.3]heptane-2-carboxylate (0.65 g, 1.4 mmol) in DCM (2 mL) were added TFA (10 mL) at room temperature. The resulting mixture was stirred for 2 h at room temperature. The reaction mixture was concentrated under reduced pressure and diluting with DCM (100 mL). The DCM was washed with NaHCO₃ (a.q. 50 mL), dried over anhydrous Na₂SO₄. After filtration, the filtrate was concentrated under reduced pressure to afford the product (0.12 g, 24% yield). [M+H]⁺=356.2.

### Step 4: 3-(4-(6-(1-(4-((5-bromo-4-((5-(dimethylphosphoryl)-2-ethylquinazolin-6-yl)amino)pyrimidin-2-yl)amino)-2-ethyl-5-methoxyphenyl)piperidin-4-yl)-2,6-diazaspiro[3.3]heptan-2-yl)-3-methyl-2-oxo-2,3-dihydro-1H-benzo[d]imidazol-1-yl)piperidine-2,6-dione

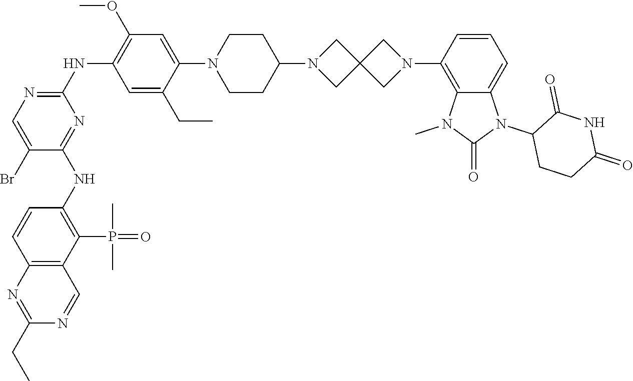 [SMILES: CCc1ncc2c(P(C)(C)=O)c(Nc3nc(Nc4cc(CC)c(N5CCC(N6CC7(CN(c8cccc9c8n(C)c(=O)n9C8CCC(=O)NC8=O)C7)C6)CC5)cc4OC)ncc3Br)ccc2n1]

A mixture of 1-(4-((5-bromo-4-((5-(dimethylphosphoryl)-2-ethylquinazolin-6-yl)amino)pyrimidin-2-yl)amino)-2-ethyl-5-methoxyphenyl)piperidin-4-one (50 mg, 0.07 mmol), 3-(3-methyl-2-oxo-4-(2,6-diazaspiro[3.3]heptan-2-yl)-2,3-dihydro-1H-benzo[d]imidazol-1-yl)piperidine-2,6-dione (27 mg, 0.07 mmol), STAB (32 mg, 0.15 mmol) and AOH (9 mg, 0.15 mmol) in DCE (10 mL) was stirred in a flask at 80° C. overnight. The reaction mixture was allowed to cool down to room temperature. The resulting mixture was diluted with DCM (100 mL), washed with brine (100 mL), dried over anhydrous Na₂SO₄. After filtration, the filtrate was concentrated under reduced pressure. Purification by flash column chromatography (0-10% MeOH in DCM) followed by prep-HPLC chromatography (0.1% FA in water:acetonitrile=90:10˜50:50 gradient elution) afforded the title compound (10.8 mg, 14.2%). ¹H NMR (500 MHz, DMSO) δ 11.59 (s, 1H), 11.08 (s, 1H), 9.87 (s, 1H), 8.46 (s, 1H), 8.24 (s, 1H), 8.04 (s, 1H), 7.86 (d, J=9.3 Hz, 1H), 7.27 (s, 1H), 6.95 (t, J=8.3 Hz, 1H), 6.73 (s, 2H), 6.64 (d, J=8.4 Hz, 1H), 5.36-5.29 (m, 1H), 3.91 (s, 3H), 3.76 (s, 3H), 3.58 (s, 3H), 3.08-3.03 (m, 2H), 2.88 (s, 4H), 2.66 (d, J=26.1 Hz, 6H), 2.23 (s, 1H), 2.03 (d, J=13.4 Hz, 7H), 1.98 (s, 1H), 1.74 (d, J=9.6 Hz, 2H), 1.37 (q, J=7.5 Hz, 4H), 1.30 (s, 2H), 1.24 (s, 2H), 0.74 (s, 3H). [M+H]⁺=991.4.

### Example 26: 3-(4-(6-(1-(4-((5-bromo-4-((5-(dimethylphosphoryl)-3-fluoro-2-methylquinolin-6-yl)amino)pyrimidin-2-yl)amino)-2-ethyl-5-methoxyphenyl)piperidin-4-yl)-2,6-diazaspiro[3.3]heptan-2-yl)-3-methyl-2-oxo-2,3-dihydro-1H-benzo[d]imidazol-1-yl)piperidine-2,6-dione

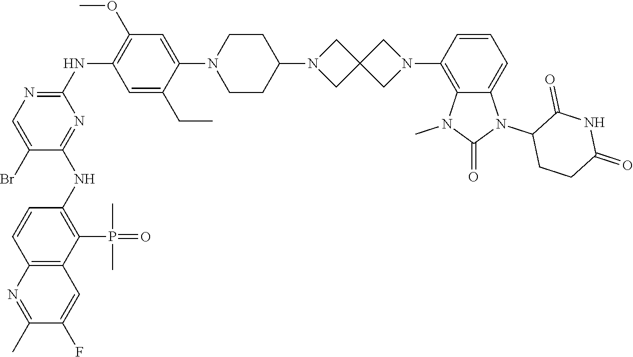 [SMILES: CCc1cc(Nc2ncc(Br)c(Nc3ccc4nc(C)c(F)cc4c3P(C)(C)=O)n2)c(OC)cc1N1CCC(N2CC3(CN(c4cccc5c4n(C)c(=O)n5C4CCC(=O)NC4=O)C3)C2)CC1]

A mixture of 1-(4-((5-bromo-4-((5-(dimethylphosphoryl)-3-fluoro-2-methylquinolin-6-yl)amino)pyrimidin-2-yl)amino)-2-ethyl-5-methoxyphenyl)piperidin-4-one (50 mg, 0.07 mmol), 3-(3-methyl-2-oxo-4-(2,6-diazaspiro[3.3]heptan-2-yl)-2,3-dihydro-1H-benzo[d]imidazol-1-yl)piperidine-2,6-dione (27 mg, 0.07 mmol), STAB (32 mg, 0.15 mmol) and AOH (9 mg, 0.15 mmol) in DCE (10 mL) was stirred in a flask at 80° C. overnight. The reaction mixture was allowed to cool down to room temperature. The resulting mixture was diluted with DCM (100 mL), washed with brine (100 mL), dried over anhydrous Na₂SO₄. After filtration, the filtrate was concentrated under reduced pressure. Purification by flash column chromatography (0-10% MeOH in DCM) followed by prep-HPLC chromatography (0.1% FA in water:acetonitrile=90:10˜50:50 gradient elution) afforded the title compound (7.8 mg, 10.3%). ¹H NMR (500 MHz, DMSO) δ 11.29 (s, 1H), 11.08 (s, 1H), 8.62 (s, 1H), 8.21 (d, J=19.3 Hz, 2H), 7.95 (d, J=10.3 Hz, 2H), 7.32 (s, 1H), 6.95 (t, J=8.0 Hz, 1H), 6.72 (d, J=18.4 Hz, 2H), 6.64 (d, J=7.1 Hz, 1H), 5.35-5.29 (m, 1H), 3.91 (s, 3H), 3.76 (s, 3H), 3.58 (s, 3H), 2.87 (d, J=11.2 Hz, 3H), 2.63 (dd, J=14.8, 11.2 Hz, 6H), 2.16 (s, 4H), 1.95 (d, J=13.4 Hz, 9H), 1.73 (d, J=19.2 Hz, 3H), 1.27 (d, J=32.2 Hz, 4H), 0.71 (s, 3H). [M+H]⁺=994.5

### Example 27: (R)-3-(4-(6-(1-(4-((5-bromo-4-((5-(dimethylphosphoryl)-2-ethylquinolin-6-yl)amino)pyrimidin-2-yl)amino)-2-ethyl-5-methoxyphenyl)piperidin-4-yl)-2,6-diazaspiro[3.3]heptan-2-yl)-2,6-difluorophenyl)piperidine-2,6-dione

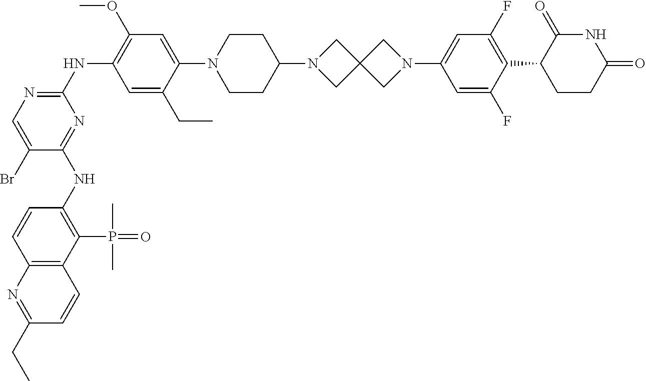 [SMILES: CCc1ccc2c(P(C)(C)=O)c(Nc3nc(Nc4cc(CC)c(N5CCC(N6CC7(CN(c8cc(F)c([C@H]9CCC(=O)NC9=O)c(F)c8)C7)C6)CC5)cc4OC)ncc3Br)ccc2n1]

A mixture of 1-(4-((5-bromo-4-((5-(dimethylphosphoryl)-2-ethylquinolin-6-yl)amino)pyrimidin-2-yl)amino)-2-ethyl-5-methoxyphenyl)piperidin-4-one (100 mg, 0.15 mmol), (R)-3-(2,6-difluoro-4-(2,6-diazaspiro[3.3]heptan-2-yl)phenyl)piperidine-2,6-dione (the intermediate was obtained through the similar way of Example 9 step 4) (49 mg, 0.15 mmol), STAB (65 mg, 0.3 mmol) and AOH (18 mg, 0.3 mmol) in DCE (10 mL) was stirred in a flask at 80° C. overnight. The reaction mixture was allowed to cool down to room temperature. The resulting mixture was diluted with DCM (100 mL), washed with brine (100 mL), dried over anhydrous Na₂SO₄. After filtration, the filtrate was concentrated under reduced pressure. The residue was purified by silica gel column chromatography (DCM:MEOH=0-10%) to afford the crude product which was purified by SFC (IH (3*25 cm, 5 um), 13% EtOH/87% CO₂, 100 bar, 100 ml/min) and the title compound corresponded to peak A @ 1.36 min/254 nm (22.9 mg, 15.6%). ¹H NMR (500 MHz, DMSO) δ 11.77 (s, 1H), 10.86 (s, 1H), 8.58 (d, J=8.6 Hz, 1H), 8.27 (s, 1H), 8.21 (s, 1H), 7.99 (s, 1H), 7.88 (d, J=9.2 Hz, 1H), 7.44 (d, J=8.9 Hz, 1H), 7.34 (s, 1H), 6.74 (s, 1H), 6.16 (s, 2H), 4.04 (dd, J=12.5, 4.9 Hz, 1H), 3.92 (s, 3H), 3.77 (s, 3H), 3.32-3.21 (m, 8H), 2.93 (dd, J=15.1, 7.5 Hz, 4H), 2.77 (dd, J=21.4, 9.2 Hz, 1H), 2.63 (t, J=9.9 Hz, 2H), 2.26 (d, J=7.1 Hz, 2H), 2.08 (d, J=12.9 Hz, 2H), 1.97 (t, J=14.6 Hz, 7H), 1.72 (s, 1H), 1.32 (t, J=7.6 Hz, 4H), 0.75 (s, 3H). [M+H]⁺=956.3.

### Example 28: 3-(4-(3-(9-(4-((5-bromo-4-((5-(dimethylphosphoryl)-3-fluoro-2-methylquinolin-6-yl)amino)pyrimidin-2-yl)amino)-2-ethyl-5-methoxyphenyl)-3,9-diazaspiro[5.5]undecan-3-yl)propyl)-3-methyl-2-oxo-2,3-dihydro-1H-benzo[d]imidazol-1-yl)piperidine-2,6-dione

### Step 1: 4-(3-(benzyloxy)propyl)-1-(2,6-bis(benzyloxy)pyridin-3-yl)-3-methyl-1,3-dihydro-2H-benzo[d]imidazol-2-one

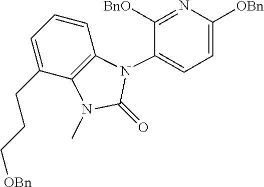 [SMILES: Cn1c(=O)n(-c2ccc(OCc3ccccc3)nc2OCc2ccccc2)c2cccc(CCCOCc3ccccc3)c21]

To a solution of 1-(2,6-bis(benzyloxy)pyridin-3-yl)-4-bromo-3-methyl-1,3-dihydro-2H-benzo[d]imidazol-2-one (1.04 g, 1.89 mmol), ((3-bromopropoxy)methyl)benzene (520 mg, 2.29 mmol) and benzamidine (60 mg, 0.38 mmol) in DMA (10 mL) were added Mn (310 mg, 5.64 mmol), nickel(II) iodide (120 mg, 0.38 mmol), NaI (140 mg, 0.93 mmol) and TFA (110 mg, 0.97 mmol) at 0° C. The mixture was stirred for 15 min at room temperature and then 2 h at 100° C. under N₂ atmosphere. The reaction was quenched with H₂O at room temperature. The resulting mixture was filtered, the filter cake was washed with EA. The filtrate was extracted with EA. The combined organic layers were washed with brine and concentrated under reduced pressure. The residue was applied onto a silica gel column with PE:EA (100:0˜75:25). This resulted in 4-(3-(benzyloxy)propyl)-1-(2,6-bis(benzyloxy)pyridin-3-yl)-3-methyl-1,3-dihydro-2H-benzo[d]imidazol-2-one (700 mg, 63.2%), ([M+1]=586.3.

### Step 2: 3-(4-(3-hydroxypropyl)-3-methyl-2-oxo-2,3-dihydro-1H-benzo[d]imidazol-1-yl)piperidine-2,6-dione

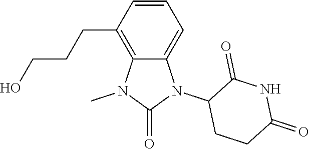 [SMILES: Cn1c(=O)n(C2CCC(=O)NC2=O)c2cccc(CCCO)c21]

Into a 50-mL round-bottom flask were placed 4-(3-(benzyloxy)propyl)-1-(2,6-bis(benzyloxy)pyridin-3-yl)-3-methyl-1,3-dihydro-2H-benzo[d]imidazol-2-one (700 mg, 1.19 mmol), THF (20 mL), Pd/C (500 mg, 10% wt). To the above H₂(g) was introduced in. The resulting solution was stirred overnight at 25 degrees C.

The solids were filtered out. The resulting mixture was concentrated under vacuum. This resulted in 270 mg (71.3%) of 3-(4-(3-hydroxypropyl)-3-methyl-2-oxo-2,3-dihydro-1H-benzo[d]imidazol-1-yl)piperidine-2,6-dione, [M+1]=318.2.

### Step 3: 3-(1-(2,6-dioxopiperidin-3-v)-3-methyl-2-oxo-2,3-dihydro-1H-benzo[d]imidazol-4-yl)propanal

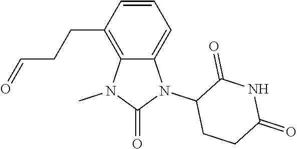 [SMILES: Cn1c(=O)n(C2CCC(=O)NC2=O)c2cccc(CCC=O)c21]

The titled compound (130 mg, 89.2%) was prepared in a manner similar to that in Example 1 step 9 from 3-(4-(3-hydroxypropyl)-3-methyl-2-oxo-2,3-dihydro-1H-benzo[d]imidazol-1-yl)piperidine-2,6-dione and DMP. [M+H]⁺=316.1.

### Step 4: 3-(4-(3-(9-(4-((5-bromo-4-((5-(dimethylphosphoryl)-3-fluoro-2-methylquinolin-6-yl)amino)pyrimidin-2-yl)amino)-2-ethyl-5-methoxyphenyl)-3,9-diazaspiro[5.5]undecan-3-yl)propyl)-3-methyl-2-oxo-2,3-dihydro-1H-benzo[d]imidazol-1-yl)piperidine-2,6-dione

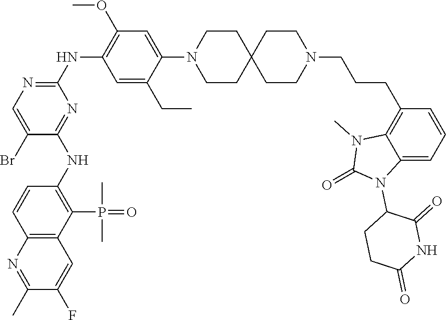 [SMILES: CCc1cc(Nc2ncc(Br)c(Nc3ccc4nc(C)c(F)cc4c3P(C)(C)=O)n2)c(OC)cc1N1CCC2(CCN(CCCc3cccc4c3n(C)c(=O)n4C3CCC(=O)NC3=O)CC2)CC1]

To a solution of (6-((5-bromo-2-((5-ethyl-2-methoxy-4-(3,9-diazaspiro[5.5]undecan-3-yl)phenyl)amino)pyrimidin-4-yl)amino)-3-fluoro-2-methylquinolin-5-yl)dimethylphosphine oxide (71 mg, 0.1 mmol) and 3-(1-(2,6-dioxopiperidin-3-yl)-3-methyl-2-oxo-2,3-dihydro-1H-benzo[d]imidazol-4-yl)propanal (38 mg, 0.12 mmol) in DCE (5 mL) was added STAB (43 mg, 0.2 mmol) at 60° C. The mixture was stirred at 60° C. for 1 hr. Water (10 mL) was poured into the mixture. Then the mixture was extracted with DCM (20 mL). The organic phase was washed with brine (10 mL), dried over Na₂SO₄, filtered and concentrated in vacuum. The residue was purified by prep-HPLC (C-18 column chromatography (0.1% FA in water:acetonitrile=90:10˜60:40 gradient elution) to afford the product (21.4 mg, 21.2%).

¹H NMR (500 MHz, DMSO) δ 11.34 (s, 1H), 11.09 (s, 1H), 8.60 (d, J=12.2 Hz, 1H), 8.22 (s, 1H), 8.19 (s, 1H), 7.96 (s, 1H), 7.94 (d, J=9.9 Hz, 1H), 7.30 (s, 1H), 6.97 (s, 1H), 6.96 (s, 1H), 6.92-6.87 (m, 1H), 6.79 (s, 1H), 5.37 (dd, J=12.6, 5.3 Hz, 1H), 3.75 (s, 3H), 3.58 (s, 3H), 2.94-2.85 (m, 3H), 2.73-2.68 (m, 5H), 2.63 (d, J=1.7 Hz, 4H), 2.41-2.36 (m, 5H), 2.20 (d, J=3.8 Hz, 2H), 2.00 (d, J=6.0 Hz, 2H), 1.97 (s, 3H), 1.94 (s, 3H), 1.82-1.74 (m, 2H), 1.56-1.49 (m, 8H), 0.72 (s, 3H). [M+H]⁺=1009.7

### Example 29: 3-((4-(1-(2-(9-(4-((5-bromo-4-((5-(dimethylphosphoryl)-3-fluoro-2-methylquinolin-6-yl)amino)pyrimidin-2-yl)amino)-2-ethyl-5-methoxyphenyl)-3,9-diazaspiro[5.5]undecan-3-yl)-2-oxoethyl)piperidin-4-yl)phenyl)amino)piperidine-2,6-dione

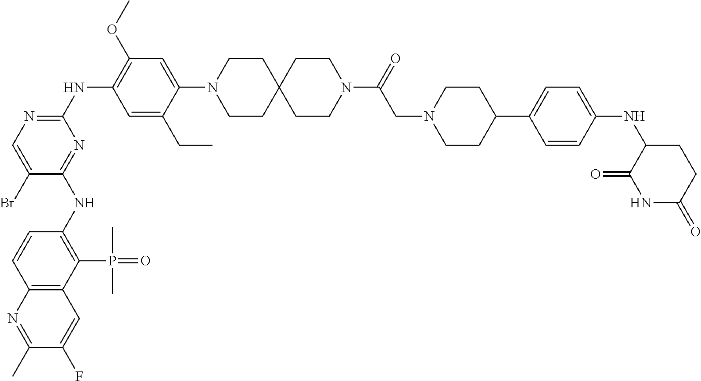 [SMILES: CCc1cc(Nc2ncc(Br)c(Nc3ccc4nc(C)c(F)cc4c3P(C)(C)=O)n2)c(OC)cc1N1CCC2(CCN(C(=O)CN3CCC(c4ccc(NC5CCC(=O)NC5=O)cc4)CC3)CC2)CC1]

To a mixture of 2-(4-(4-((2,6-dioxopiperidin-3-yl)amino)phenyl)piperidin-1-yl)acetic acid (This intermediate was prepared according to the same manner as described in WO 2021178920 A1) (48 mg, 0.139 mmol) and HATU (58.1 mg, 0.153 mmol) in DMF (3 mL) was added DIEA (25 mg, 0.190 mmol). The mixture was stirred at room temperature for 15 mins. (6-((5-bromo-2-((5-ethyl-2-methoxy-4-(3,9-diazaspiro[5.5]undecan-3-yl)phenyl)amino)pyrimidin-4-yl)amino)-3-fluoro-2-methylquinolin-5-yl)dimethylphosphine oxide (90 mg, 0.127 mmol) was added and the mixture was stirred for 2 hours. The mixture was concentrated in vacuum and purified by prep-HPLC chromatography (0.1% FA in water:acetonitrile=90:10-50:50 gradient elution) afforded the racemic compound, which was purified by SFC (IF (2*25 cm, 5 um), Phase A: METB, Phase B: MeOH/DCM, 20 ml/min, UV 220 nm) and the title compound corresponded to peak A @ 1.547 min/254 nm (24.09 mg, 17.84%). ¹H NMR (500 MHz, DMSO) δ 11.33 (s, 1H), 10.76 (s, 1H), 8.61 (d, J=12.5 Hz, 1H), 8.22 (s, 2H), 7.95 (d, J=14.2 Hz, 2H), 7.31 (s, 1H), 6.96 (d, J=8.1 Hz, 2H), 6.81 (s, 1H), 6.61 (d, J=8.2 Hz, 2H), 5.68 (s, 1H), 4.26 (s, 1H), 3.76 (s, 3H), 3.50 (s, 4H), 2.81-2.71 (m, 6H), 2.68-2.52 (m, 6H), 2.42-2.35 (m, 2H), 2.26-2.15 (m, 2H), 2.15-2.08 (m, 2H), 1.95 (d, J=13.3 Hz, 6H), 1.90-1.82 (m, 2H), 1.81-1.71 (m, 2H), 1.65-1.52 (m, 8H), 1.47 (s, 2H), 0.72 (s, 3H); [M+H]⁺=1037.5.

### Example 30: (R)-3-(4-(1-(1′-(4-((5-bromo-4-((5-(dimethylphosphoryl)-2-ethylquinolin-6-yl)amino)pyrimidin-2-yl)amino)-2-ethyl-5-methoxyphenyl)-[1,4′-bipiperidin]-4-yl)azetidin-3-yl)-2,6-difluorophenyl)piperidine-2,6-dione

### Step 1: tert-butyl 3-(4-(2,6-bis(benzyloxy)pyridin-3-yl)-3,5-difluorophenyl)azetidine-1-carboxylate

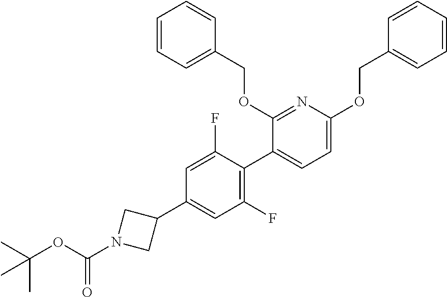 [SMILES: CC(C)(C)OC(=O)N1CC(c2cc(F)c(-c3ccc(OCc4ccccc4)nc3OCc3ccccc3)c(F)c2)C1]

To a solution of 2,6-bis(benzyloxy)-3-(4-bromo-2,6-difluorophenyl)pyridine (7.0 g, 14.5 mmol) in DMA (70 mL) were added tert-butyl 3-iodoazetidine-1-carboxylate (4.9 g, 17.3 mmol), pyridine-2-carboximidamide hydrochloride (0.5 g, 3.2 mmol), NiI₂ (0.9 g, 2.9 mmol), NaI (1.1 g, 7.3 mmol) and Mn (2.4 g, 43.7 mmol). Then to this was added the solution of TFA (0.8 g, 7.0 mmol) in DMA (1 mL) dropwise in 5 min under nitrogen atmosphere. The resulting solution was stirred for 5 min at 25° C. under nitrogen atmosphere. Then the reaction was stirred for 3 h at 100° C. under nitrogen atmosphere. The resulting mixture was concentrated under reduced pressure. The residue was purified by a silica gel column, eluted with PE/EtOAc (2:1) to afford the product (2.2 g, 27.2%). [M+H]⁺=559.2.

### Step 2: tert-butyl 3-(4-(2,6-dioxopiperidin-3-yl)-3,5-difluorophenyl)azetidine-1-carboxylate

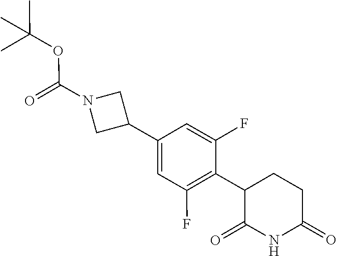 [SMILES: CC(C)(C)OC(=O)N1CC(c2cc(F)c(C3CCC(=O)NC3=O)c(F)c2)C1]

To a solution of tert-butyl 3-(4-(2,6-bis(benzyloxy)pyridin-3-yl)-3,5-difluorophenyl)azetidine-1-carboxylate (2.2 g, 3.9 mmol) in EtOH (30 mL) and DCM (6 mL) was added Pd/C (2.2 g, 10% wt). The resulting solution was stirred for 8 h at 40° C. under H₂ atmosphere. After filtration, the filtrate was concentrated under reduced pressure. This was resulted in tert-butyl 3-(4-(2,6-dioxopiperidin-3-yl)-3,5-difluorophenyl)azetidine-1-carboxylate (1.4 g, 93.6%). ¹H NMR (500 MHz, DMSO) δ 10.95 (s, 1H), 7.16-7.11 (m, 2H), 4.25-4.19 (m, 3H), 3.87-3.85 (m, 3H), 3.46-3.42 (m, 1H), 2.87-2.76 (m, 1H), 2.22-1.95 (m, 2H), 1.40 (s, 9H). [M+H]⁺=381.2.

### Step 3: 3-(4-(azetidin-3-yl)-2,6-difluorophenyl)piperidine-2,6-dione hydrochloride

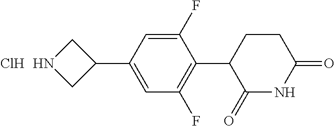 [SMILES: Cl ; O=C1CCC(c2c(F)cc(C3CNC3)cc2F)C(=O)N1]

To a solution of tert-butyl 3-(4-(2,6-dioxopiperidin-3-yl)-3,5-difluorophenyl)azetidine-1-carboxylate (50 mg, 0.131 mmol) in dioxane (1 mL) was added 4 M of hydrochloride in dioxane (3 mL), stirred for 2 hours at room temperature. The resulting mixture was concentrated, dried over in vacuo to afford 3-(4-(azetidin-3-yl)-2,6-difluorophenyl)piperidine-2,6-dione hydrochloride (41 mg crude). [M+H]⁺=281.1.

### Step 4: (R)-3-(4-(1-(1′-(4-((5-bromo-4-((5-(dimethylphosphoryl)-2-ethylquinolin-6-yl)amino)pyrimidin-2-yl)amino)-2-ethyl-5-methoxyphenyl)-[1,4′-bipiperidin]-4-yl)azetidin-3-yl)-2,6-difluorophenyl)piperidine-2,6-dione

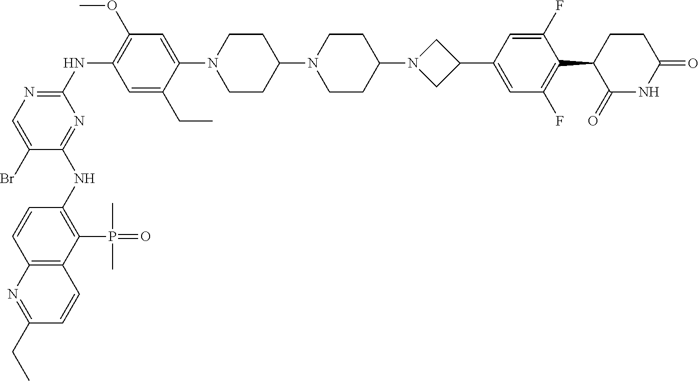 [SMILES: CCc1ccc2c(P(C)(C)=O)c(Nc3nc(Nc4cc(CC)c(N5CCC(N6CCC(N7CC(c8cc(F)c([C@H]9CCC(=O)NC9=O)c(F)c8)C7)CC6)CC5)cc4OC)ncc3Br)ccc2n1]

To a suspension of 1′-(4-((5-bromo-4-((5-(dimethylphosphoryl)-2-ethylquinolin-6-yl)amino)pyrimidin-2-yl)amino)-2-ethyl-5-methoxyphenyl)-[1,4′-bipiperidin]-4-one (80 mg, 0.109 mmol) and 3-(4-(azetidin-3-yl)-2,6-difluorophenyl)piperidine-2,6-dione hydrochloride (41 mg, 0.131 mmol) in dichloromethane/methanol (8 mL/4 mL), N,N-diethylpropylethylamine (40 mg, 0.31 mmol) was added, stirred for 20 min, acetic acid (24 mg, 0.4 mmol) was added, stirred for 20 min, NaBH₃CN (20 mg, 0.32 mmol) was then added, stirred for another 2 hours at room temperature. After LCMS showed the reaction was completed. The mixture was diluted with DCM, washed with brine, dried over anhydrous sodium sulfate, filtered, and concentrated under vacuum. The residue was purified by flash column chromatography with 0-25% MeOH in DCM and Prep-HPLC chromatography (0.1% FA in water:acetonitrile=90:10˜50:50 gradient elution) to afford the product which was further purified by SFC by chiral-HPLC (CHIRALPAK IF-3 4.6*50 mm, 3 μm), MtBE (0.1% DEA):(MeOH:DCM=1:1)=50:50, 1.5 ml/min) and the title compound corresponded to peak A @ 1.65 min/254 nm) to afford the desired enantiomer (20 mg, 18%). ¹H NMR (500 MHz, DMSO) δ=11.80 (s, 1H), 10.96 (s, 1H), 8.55 (d, J=9.0, 1H), 8.27 (s, 1H), 8.21 (s, 1H), 8.00 (s, 1H), 7.87 (d, J=9.3, 1H), 7.44 (d, J=8.9, 1H), 7.33 (s, 1H), 7.12 (d, J=10.1, 2H), 6.75 (s, 1H), 4.21 (dd, J=12.6, 4.9, 1H), 3.76 (s, 3H), 3.61-3.51 (m, 3H), 3.06 (m, 2H), 2.93 (m, 4H), 2.88-2.75 (m, 3H), 2.66 (t, J=11.4, 2H), 2.55 (m, 1H), 2.41-2.07 (m, 6H), 1.98 (d, J=13.3, 7H), 1.80 (d, J=10.8, 2H), 1.69-1.52 (m, 4H), 1.32 (t, J=7.6, 3H), 1.20 (m, 3H), 0.77 (s, 3H). [M+H]⁺=998.7.

### Example 31: 3-(4-(2-(1-(4-((5-bromo-4-((5-(dimethylphosphoryl)-2-ethylquinazolin-6-yl)amino)pyrimidin-2-yl)amino)-2-ethyl-5-methoxyphenyl)piperidin-4-yl)-2,7-diazaspiro[3.5]nonan-7-yl)-3-methyl-2-oxo-2,3-dihydro-1H-benzol[d]imidazol-1-yl)piperidine-2,6-dione

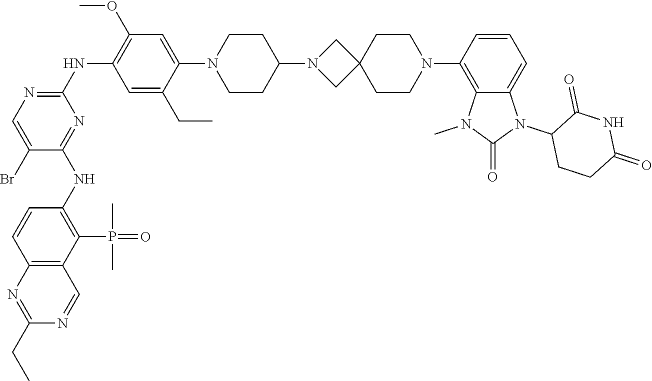 [SMILES: CCc1ncc2c(P(C)(C)=O)c(Nc3nc(Nc4cc(CC)c(N5CCC(N6CC7(CCN(c8cccc9c8n(C)c(=O)n9C8CCC(=O)NC8=O)CC7)C6)CC5)cc4OC)ncc3Br)ccc2n1]

A mixture of 1-(4-((5-bromo-4-((5-(dimethylphosphoryl)-2-ethylquinazolin-6-yl)amino)pyrimidin-2-yl)amino)-2-ethyl-5-methoxyphenyl)piperidin-4-one (50 mg, 0.08 mmol), 3-(3-methyl-2-oxo-4-(2,7-diazaspiro[3.5]nonan-7-yl)-2,3-dihydro-1H-benzo[d]imidazol-1-yl)piperidine-2,6-dione (the intermediate was obtained through the similar way of Example 25 step 3) (29 mg, 0.08 mmol), STAB (32 mg, 0.15 mmol) and AOH (9 mg, 0.15 mmol) in DCE (10 mL) was stirred in a flask at 80° C. overnight. The reaction mixture was allowed to cool down to room temperature. The resulting mixture was diluted with DCM (100 mL), washed with brine (100 mL), dried over anhydrous Na₂SO₄. After filtration, the filtrate was concentrated under reduced pressure. Purification by flash column chromatography (0-10% MeOH in DCM) followed by prep-HPLC chromatography (0.1% FA in water:acetonitrile=90:10˜50:50 gradient elution) afforded the title compound (28 mg, 35.8%). ¹H NMR (500 MHz, DMSO) δ 11.60 (s, 1H), 11.08 (s, 1H), 9.86 (s, 1H), 8.45 (s, 1H), 8.24 (s, 1H), 8.05 (s, 1H), 7.86 (d, J=9.3 Hz, 1H), 7.27 (s, 1H), 6.97 (t, J=8.0 Hz, 1H), 6.88 (dd, J=13.3, 7.9 Hz, 2H), 6.74 (s, 1H), 5.35 (dd, J=12.7, 5.3 Hz, 1H), 3.76 (s, 3H), 3.63 (s, 3H), 3.12-2.85 (m, 11H), 2.75-2.58 (m, 6H), 2.23 (s, 3H), 2.01 (dd, J=21.5, 9.3 Hz, 7H), 1.87 (d, J=20.5 Hz, 4H), 1.75 (d, J=9.4 Hz, 2H), 1.38 (t, J=7.6 Hz, 3H), 1.32 (d, J=10.2 Hz, 2H), 0.75 (s, 3H). [M+H]⁺=1019.5

### Example 32: 3-(4-(2-(1-(4-((5-chloro-4-((5-(dimethylphosphoryl)-3-fluoro-2-methylquinolin-6-yl)amino)pyrimidin-2-yl)amino)-5-ethoxy-2-ethylphenyl)piperidin-4-yl)-2,7-diazaspiro[3.5]nonan-7-yl)-3-methyl-2-oxo-2,3-dihydro-1H-benzol[d]imidazol-1-yl)piperidine-2,6-dione

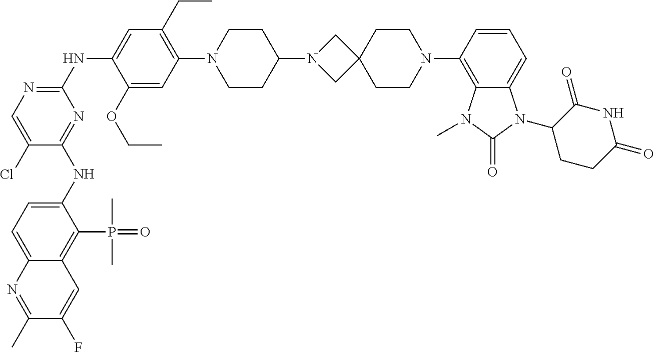 [SMILES: CCOc1cc(N2CCC(N3CC4(CCN(c5cccc6c5n(C)c(=O)n6C5CCC(=O)NC5=O)CC4)C3)CC2)c(CC)cc1Nc1ncc(Cl)c(Nc2ccc3nc(C)c(F)cc3c2P(C)(C)=O)n1]

A mixture of 1-(4-((5-chloro-4-((5-(dimethylphosphoryl)-3-fluoro-2-methylquinolin-6-yl)amino)pyrimidin-2-yl)amino)-5-ethoxy-2-ethylphenyl)piperidin-4-one (50 mg, 0.08 mmol), 3-(3-methyl-2-oxo-4-(2,7-diazaspiro[3.5]nonan-7-yl)-2,3-dihydro-1H-benzo[d]imidazol-1-yl)piperidine-2,6-dione (31 mg, 0.08 mmol), STAB (34 mg, 0.16 mmol) and AOH (10 mg, 0.16 mmol) in DCE (10 mL) was stirred in a flask at 80° C. overnight. The reaction mixture was allowed to cool down to room temperature. The resulting mixture was diluted with DCM (100 mL), washed with brine (100 mL), dried over anhydrous Na₂SO₄. After filtration, the filtrate was concentrated under reduced pressure. Purification by flash column chromatography (0-10% MeOH in DCM) followed by prep-HPLC chromatography (0.1% FA in water:acetonitrile=90:10 50:50 gradient elution) afforded the title compound (48 mg, 60.3%). ¹H NMR (500 MHz, DMSO) δ 11.54 (s, 1H), 11.09 (s, 1H), 8.63 (d, J=12.1 Hz, 1H), 8.31 (s, 1H), 8.17 (s, 1H), 7.95 (d, J=9.2 Hz, 1H), 7.89 (s, 1H), 7.39 (s, 1H), 6.97 (t, J=7.9 Hz, 1H), 6.91-6.82 (m, 2H), 6.69 (s, 1H), 5.35 (dd, J=12.5, 5.3 Hz, 1H), 4.01 (q, J=6.9 Hz, 2H), 3.63 (s, 3H), 3.23 (s, 2H), 3.11 (s, 2H), 3.00 (s, 2H), 2.89 (t, J=10.9 Hz, 3H), 2.76-2.53 (m, 9H), 2.36 (s, 1H), 2.18 (d, J=6.0 Hz, 2H), 2.03-1.73 (m, 13H), 1.34 (d, J=9.9 Hz, 2H), 1.27 (t, J=6.9 Hz, 3H), 0.71 (s, 3H). [M+H]⁺=992.8.

### Example 33: 3-(4-(3-((9-(4-((5-bromo-4-((5-(dimethylphosphoryl)-3-fluoro-2-methylquinolin-6-yl)amino)pyrimidin-2-yl)amino)-2-ethyl-5-methoxyphenyl)-3,9-diazaspiro[5.5]undecan-3-yl)methyl)azetidin-1-yl)-1-oxoisoindolin-2-yl)piperidine-2,6-dione

### Step 1: N-(2,6-bis(benzyloxy)pyridin-3-yl)-1,1-diphenylmethanimine

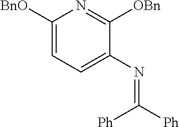 [SMILES: c1ccc(COc2ccc(N=C(c3ccccc3)c3ccccc3)c(OCc3ccccc3)n2)cc1]

A mixture of 2,6-bis(benzyloxy)-3-bromopyridine (60 g, 162.1 mmol) and diphenylmethanimine (35.2 g, 194.5 mmol), Cs₂CO₃ (105.6 g, 324.1 mmol), BINAP (10.1 g, 16.2 mmol), Pd₂(dba)₃ (14.8 g, 16.2 mmol) in dioxane (600 mL) was stirred overnight at 100° C. under nitrogen atmosphere. The resulting mixture was concentrated under reduced pressure. The residue was purified by silica gel column chromatography, eluted with PE/EtOAc (10:1) to afford the desired product (48 g, 62.9%). [M+H]⁺=471.3.

### Step 2: 2,6-bis(benzyloxy)pyridin-3-amine

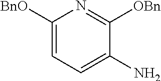 [SMILES: Nc1ccc(OCc2ccccc2)nc1OCc1ccccc1]

A mixture of N-(2,6-bis(benzyloxy)pyridin-3-yl)-1,1-diphenylmethanimine (48 g, 102.0 mmol) in THF (300 mL) and 1 N HCl (360 mL) was stirred overnight at rt. The resulting mixture was concentrated under vacuum. The precipitated solids were collected by filtration and washed with water (500 mL) and sat. aq.

NaHCO₃ solution. The residue was purified by trituration with EtOAc:PE (1:10) for 3 times to afford the product. (25 g, 80.00%). [M+H]⁺=307.1.

### Step 3: 2-(2,6-bis(benzyloxy)pyridin-3-yl)-4-bromoisoindolin-1-one

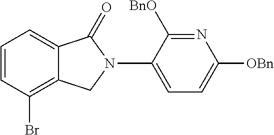 [SMILES: O=C1c2cccc(Br)c2CN1c1ccc(OCc2ccccc2)nc1OCc1ccccc1]

A mixture of 2,6-bis(benzyloxy)pyridin-3-amine (5 g, 16.3 mmol) and methyl 3-bromo-2-(bromomethyl)benzoate (5 g, 16.3 mmol), Cs₂CO₃ (16 g, 49.0 mmol) in DMF (50 mL) was stirred overnight at 100° C. The resulting mixture was extracted with EA (250 mL). The combined organic layers were washed with brine (500 mL), dried over anhydrous Na₂SO₄. After filtration, the filtrate was concentrated under reduced pressure. The residue was purified by silica gel column chromatography, eluted with PE/EtOAc (1:1) to afford the desired product (4.2 g, 51.33%). [M+H]⁺=501.2.

### Step 4: 4-(3-((benzyloxy)methyl)azetidin-1-yl)-2-(2,6-bis(benzyloxy)pyridin-3-yl)isoindolin-1-one

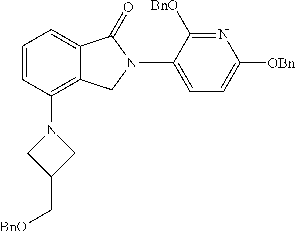 [SMILES: O=C1c2cccc(N3CC(COCc4ccccc4)C3)c2CN1c1ccc(OCc2ccccc2)nc1OCc1ccccc1]

To a solution of 2-(2,6-bis(benzyloxy)pyridin-3-yl)-4-bromoisoindolin-1-one (1 g, 2.0 mmol) in dioxane (20 mL) were added 3-((benzyloxy) methyl)azetidine (460 mg, 2.6 mmol), Ruphos (0.2 g, 0.4 mmol), Pd₂(dba)₃ (0.2 g, 0.2 mmol) and Cs₂CO₃ (3.3 g, 10.2 mmol). The resulting solution was stirred for 3 h at 100° C. under nitrogen atmosphere. The resulting mixture was concentrated under reduced pressure. The residue was purified by a silica gel column, eluted with PE/EtOAc (2:1) to afford the desired product (1.1 g, 91.7%). [M+H]⁺=598.2.

### Step 5: 3-(4-(3-(hydroxymethyl)azetidin-1-yl)-1-oxoisoindolin-2-yl)piperidine-2,6-dione

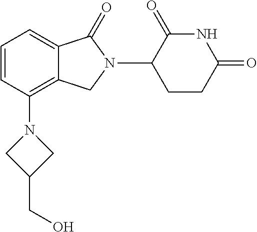 [SMILES: O=C1CCC(N2Cc3c(cccc3N3CC(CO)C3)C2=O)C(=O)N1]

To a solution of 4-(3-((benzyloxy)methyl)azetidin-1-yl)-2-(2,6-bis(benzyloxy)pyridin-3-yl)isoindolin-1-one (1 g, 1.7 mmol) in EtOAc (30 mL) were added Pd/C (1 g, 10% wt) and CH₃COOH (0.3 mL). The resulting solution was stirred for 12 h at 50° C. under H₂ atmosphere. After filtration, the filtrate was concentrated under reduced pressure to afford the pure product (0.5 g, 90.9%). [M+H]⁺=330.2.

### Step 6: 1-(2-(2,6-dioxopiperidin-3-yl)-1-oxoisoindolin-4-yl)azetidine-3-carbaldehyde

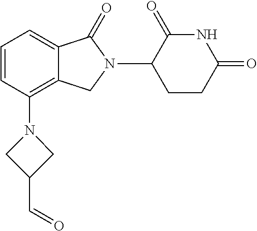 [SMILES: O=CC1CN(c2cccc3c2CN(C2CCC(=O)NC2=O)C3=O)C1]

A mixture of 3-(4-(3-(hydroxymethyl)azetidin-1-yl)-1-oxoisoindolin-2-yl)piperidine-2,6-dione (200 mg, 0.607 mmol) and DMP (514.9 mg, 1.21 mmol) in DCM (10 mL) was stirred in a flask at room temperature for 2 hrs. The reaction was quenched with water and the mixture was extracted with DCM (20 mL×3). The combined organic layers were washed with sat. aq. NaCl (30 mL×3) and sat. aq. NaHCO₃ (30 mL×2). The organic layer was dried over anhydrous Na₂SO₄ and evaporated under vacuum to afford the product (140 mg, 70.5%). [M+H]⁺=328.1.

### Step 7: 3-(4-(3-((9-(4-((5-bromo-4-((5-(dimethylphosphoryl)-3-fluoro-2-methylquinolin-6-yl)amino)pyrimidin-2-yl)amino)-2-ethyl-5-methoxyphenyl)-3,9-diazaspiro[5.5]undecan-3-yl)methyl)azetidin-1-yl)-1-oxoisoindolin-2-yl)piperidine-2,6-dione

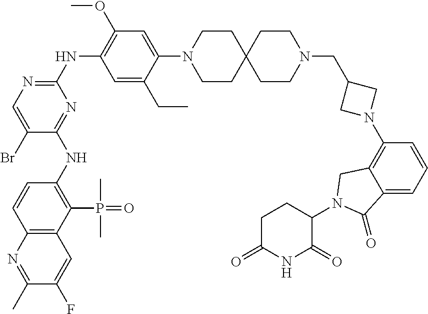 [SMILES: CCc1cc(Nc2ncc(Br)c(Nc3ccc4nc(C)c(F)cc4c3P(C)(C)=O)n2)c(OC)cc1N1CCC2(CCN(CC3CN(c4cccc5c4CN(C4CCC(=O)NC4=O)C5=O)C3)CC2)CC1]

To a solution of (6-((5-bromo-2-((5-ethyl-2-methoxy-4-(3,9-diazaspiro[5.5]undecan-3-yl)phenyl)amino)pyrimidin-4-yl)amino)-3-fluoro-2-methylquinolin-5-yl)dimethylphosphine oxide (50 mg, 0.0705 mmol) and 1-(2-(2,6-dioxopiperidin-3-yl)-1-oxoisoindolin-4-yl)azetidine-3-carbaldehyde (23.06 mg, 0.0705 mmol) in DCM (2 mL) was added NaBH(OAc)₃ (44.82 mg, 0.2115 mmol) at 20° C. The resulting mixture was stirred at room temperature for 1 hour. The reaction was quenched with water (10 mL) and the layers were separated. The aqueous layer was extracted with DCM (3×10 mL). The combined organic layers were dried over anhydrous Na₂SO₄, filtered and evaporated in vacuum to afford the crude residue, which was purified by silica gel column chromatography (DCM:MeOH=100:0˜10:1 gradient elution) to give an impure product, which was further purified with prep-HPLC (C-18 column chromatography (0.1% FA in water:acetonitrile=90:10˜60:40 gradient elution) to give the desired product (40 mg, 55.63%). ¹H NMR (500 MHz, DMSO) δ 11.34 (s, 1H), 10.97 (s, 1H), 8.60 (d, J=11.0 Hz, 1H), 8.22 (s, 2H), 7.95 (d, J=6.0 Hz, 2H), 7.31 (t, J=7.5 Hz, 2H), 7.04 (d, J=7.4 Hz, 1H), 6.78 (s, 1H), 6.54 (d, J=7.9 Hz, 1H), 5.17-5.03 (m, 1H), 4.42 (s, 1H), 4.31 (s, 1H), 4.14-4.01 (m, 2H), 3.76 (s, 3H), 3.64 (d, J=6.1 Hz, 2H), 2.90-2.92 (m, 2H), 2.71 (s, 4H), 2.64 (s, 4H), 2.57-2.59 (m, 4H), 2.38 (s, 4H), 2.19 (s, 2H), 1.95 (d, J=13.3 Hz, 7H), 1.52 (s, 7H), 0.71 (s, 3H). [M+H]⁺=1021.4.

### Example 34: 3-(4-((2-(9-(4-((5-bromo-4-((5-(dimethylphosphoryl)-3-fluoro-2-methylquinolin-6-yl)amino)pyrimidin-2-yl)amino)-2-ethyl-5-methoxyphenyl)-3,9-diazaspiro[5.5]undecan-3-yl)ethyl)(methyl)amino)-1-oxoisoindolin-2-yl)piperidine-2,6-dione

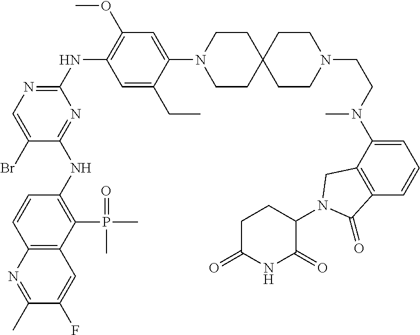 [SMILES: CCc1cc(Nc2ncc(Br)c(Nc3ccc4nc(C)c(F)cc4c3P(C)(C)=O)n2)c(OC)cc1N1CCC2(CCN(CCN(C)c3cccc4c3CN(C3CCC(=O)NC3=O)C4=O)CC2)CC1]

The title compound was prepared in a manner similar to that in Example 33. ¹H NMR (500 MHz, DMSO)¹H NMR (500 MHz, DMSO) δ 11.33 (s, 1H), 10.99 (s, 1H), 8.60 (d, J=12.5 Hz, 1H), 8.19-9.17 (m, 2H), 7.94 (d, J=12.9 Hz, 2H), 7.37 (t, J=7.7 Hz, 1H), 7.30 (s, 1H), 7.15 (d, J=7.3 Hz, 1H), 7.01 (d, J=7.9 Hz, 1H), 6.77 (s, 1H), 5.12 (dd, J=13.2, 4.9 Hz, 1H), 4.52-4.49 (m, 1H), 4.42-4.32 (m, 1H), 3.75 (s, 3H), 2.99-2.85 (m, 4H), 2.63 (s, 9H), 2.42-2.39 (m, 7H), 2.18 (s, 2H), 1.95-1.91 (m, 8H), 1.48-1.42 (m, 8H), 0.71 (s, 3H); [M+H]⁺=1009.5.

### Example 35: 3-(5-(4-((4-(1-(4-((5-bromo-4-((5-(dimethylphosphoryl)-2-ethylquinazolin-6-yl)amino)pyrimidin-2-yl)amino)-2-ethyl-5-methoxyphenyl)piperidin-4-yl)piperazin-1-yl)methyl)piperidin-1-yl)-1-oxoisoindolin-2-yl)piperidine-2,6-dione

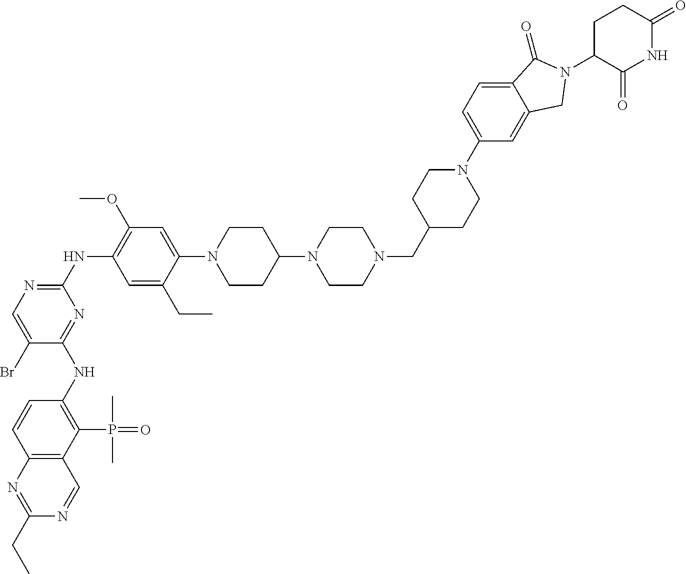 [SMILES: CCc1ncc2c(P(C)(C)=O)c(Nc3nc(Nc4cc(CC)c(N5CCC(N6CCN(CC7CCN(c8ccc9c(c8)CN(C8CCC(=O)NC8=O)C9=O)CC7)CC6)CC5)cc4OC)ncc3Br)ccc2n1]

To a suspension of (6-((5-bromo-2-((5-ethyl-2-methoxy-4-(4-(piperazin-1-yl)piperidin-1-yl)phenyl)amino)pyrimidin-4-yl)amino)-2-ethylquinazolin-5-yl)dimethylphosphine oxide (80.0 mg, 0.11 mmol) and (1-(2-(2,6-dioxopiperidin-3-yl)-1-oxoisoindolin-5-yl)piperidin-4-yl)methyl methanesulfonate (This intermediate was prepared according to the way shown in WO 2021219070 A1) (71.8 mg, 0.165 mmol) in acetonitrile (2 mL) and DMSO (0.5 mL) was added NN-diethylpropylethylamine (43 mg, 0.33 mmol) and KI (55 mg, 0.33 mmol). After stirring for 15 h at 80° C., the reaction mixture was diluted with brine (10 mL) and the layers were separated. The aqueous layer was extracted with DCM (3×10 mL). The combined organic layers were washed with brine (15 mL), dried over anhydrous Na₂SO₄, filtered and concentrated under reduced pressure. Purification by flash column chromatography (0-10% MeOH in DCM) followed by prep-HPLC chromatography (0.1% NH₄OH in water:acetonitrile=90:10˜50:50 gradient elution) afforded the title compound (35 mg, 29%). ¹H NMR (500 MHz, DMSO) δ 11.61 (s, 1H), 10.92 (s, 1H), 9.85 (s, 1H), 8.45 (s, 1H), 8.23 (s, 1H), 8.05 (s, 1H), 7.85 (d, J=9.1 Hz, 1H), 7.48 (d, J=8.8 Hz, 1H), 7.27 (s, 1H), 6.74 (s, 1H), 6.61 (d, J=7.6 Hz, 2H), 5.03 (dd, J=13.1, 5.1 Hz, 1H), 4.30 (d, J=16.3 Hz, 1H), 4.18 (d, J=16.9 Hz, 1H), 3.75 (s, 3H), 3.50 (s, 1H), 3.40 (s, 1H), 3.30-3.26 (m, 1H), 3.05 (q, J=7.5 Hz, 2H), 2.93 (d, J=9.4 Hz, 3H), 2.88 (d, J=13.4 Hz, 1H), 2.65 (d, J=10.7 Hz, 2H), 2.57 (d, J=25.9 Hz, 5H), 2.38 (s, 3H), 2.36 (s, 4H), 2.27 (d, J=8.5 Hz, 4H), 2.15 (s, 1H), 2.03 (d, J=13.4 Hz, 6H), 1.95 (d, J=5.2 Hz, 1H), 1.84 (d, J=10.6 Hz, 2H), 1.66 (d, J=8.7 Hz, 1H), 1.60 (d, J=6.9 Hz, 2H), 1.54 (d, J=9.7 Hz, 2H), 1.37 (t, J=7.5 Hz, 3H), 0.76 (s, 3H); [M+H]⁺=1061.5.

### Example 36: (R)-3-(4-(4-(9-(4-((5-bromo-4-((5-(dimethylphosphoryl)quinoxalin-6-yl)amino)pyrimidin-2-yl)amino)-5-methoxy-2-methylphenyl)-3,9-diazaspiro [5.5]undecan-3-yl)piperidin-1-yl)-2,6-difluorophenyl)piperidine-2,6-dione

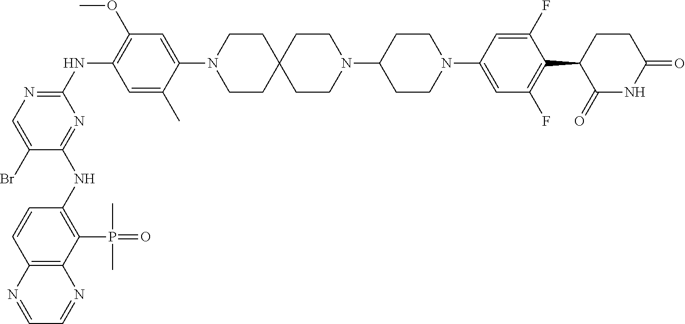 [SMILES: COc1cc(N2CCC3(CC2)CCN(C2CCN(c4cc(F)c([C@H]5CCC(=O)NC5=O)c(F)c4)CC2)CC3)c(C)cc1Nc1ncc(Br)c(Nc2ccc3nccnc3c2P(C)(C)=O)n1]

The title compound was prepared in a manner similar to that in Example 1. H NMR (500 MHz, DMSO) δ 12.75 (s, 1H), 10.89 (s, 1H), 8.88 (s, 1H), 8.86 (s, 2H), 8.38 (s, 1H), 8.29 (s, 1H), 7.93 (d, J=8.8 Hz, 1H), 7.37 (s, 1H), 6.84 (s, 1H), 6.73 (d, J=12.7 Hz, 2H), 4.07 (dd, J=12.5, 4.8 Hz, 1H), 3.98 (d, J=12.9 Hz, 2H), 3.79 (s, 3H), 3.42 (d, J=10.8 Hz, 3H), 3.12 (d, J=11.1 Hz, 2H), 2.89-2.77 (m, 6H), 2.11 (s, 6H), 2.00 (dd, J=33.6, 11.0 Hz, 10H), 1.79 (s, 2H), 1.72-1.41 (m, 7H). [M+H]⁺=971.8.

### Example 37: (R)-3-(4-(4-(9-(4-((5-bromo-4-((5-(dimethylphosphoryl)-3-fluoro-2-methylquinolin-6-yl)amino)pyrimidin-2-yl)amino)-5-methoxy-2-methylphenyl)-3,9-diazaspiro[5.5]undecan-3-yl)piperidin-1-yl)-2,6-difluorophenyl)piperidine-2,6-dione

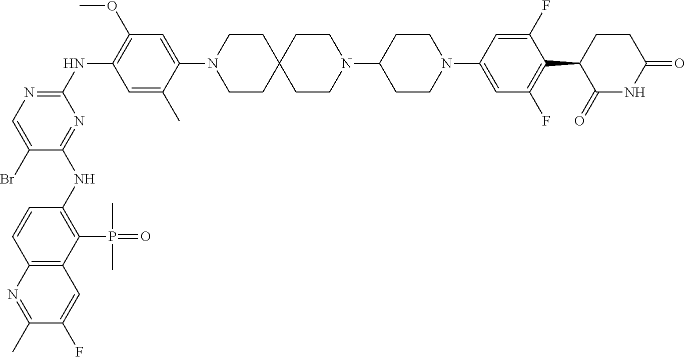 [SMILES: COc1cc(N2CCC3(CC2)CCN(C2CCN(c4cc(F)c([C@H]5CCC(=O)NC5=O)c(F)c4)CC2)CC3)c(C)cc1Nc1ncc(Br)c(Nc2ccc3nc(C)c(F)cc3c2P(C)(C)=O)n1]

The title compound was prepared in a manner similar to that in Example 1. ¹H NMR (500 MHz, DMSO) δ 11.58 (s, 1H), 10.88 (s, 1H), 8.96 (s, 1H), 8.60 (d, J=11.1 Hz, 1H), 8.26 (s, 2H), 7.97 (d, J=9.3 Hz, 1H), 7.29 (s, 1H), 6.73 (d, J=12.7 Hz, 3H), 4.07 (dd, J=12.8, 5.1 Hz, 1H), 3.98 (d, J=12.6 Hz, 2H), 3.77 (s, 3H), 3.39 (s, 3H), 3.09 (d, J=11.8 Hz, 2H), 2.87-2.73 (m, 6H), 2.64 (d, J=2.2 Hz, 3H), 2.51 (s, 2H), 2.10 (d, J=11.8 Hz, 3H), 1.96 (d, J=13.4 Hz, 9H), 1.81 (s, 5H), 1.65 (d, J=11.0 Hz, 2H), 1.54 (d, J=12.0 Hz, 4H). [M+H]⁺=1002.8.

### Example 38: (R)-3-(4-(3-(9-(4-((5-bromo-4-((5-(dimethylphosphoryl)-3-fluoro-2-methylquinolin-6-yl)amino)pyrimidin-2-yl)amino)-2-ethyl-5-methoxyphenyl)-3,9-diazaspiro[5.5]undecan-3-yl)azetidin-1-yl)-2,6-difluorophenyl)piperidine-2,6-dione

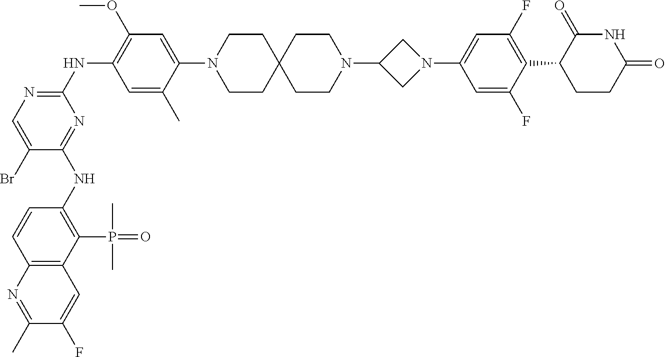 [SMILES: COc1cc(N2CCC3(CC2)CCN(C2CN(c4cc(F)c([C@H]5CCC(=O)NC5=O)c(F)c4)C2)CC3)c(C)cc1Nc1ncc(Br)c(Nc2ccc3nc(C)c(F)cc3c2P(C)(C)=O)n1]

The title compound was prepared in a manner similar to that in Example 1. H NMR (500 MHz, DMSO) δ 11.33 (s, 1H), 10.86 (s, 1H), 8.60 (d, J=12.3 Hz, 1H), 8.22 (s, 2H), 8.08-7.87 (m, 2H), 7.30 (s, 1H), 6.79 (s, 1H), 6.12 (d, J=11.3 Hz, 2H), 4.03 (dd, J=12.2, 4.8 Hz, 1H), 3.92 (t, J=7.0 Hz, 2H), 3.76 (s, 3H), 3.61 (s, 3H), 3.26 (d, J=5.9 Hz, 1H), 2.82-2.68 (m, 6H), 2.64 (s, 3H), 2.31 (s, 4H), 2.20 (s, 2H), 2.12-2.04 (m, 1H), 1.95 (d, J=13.3 Hz, 6H), 1.54 (s, 8H), 0.72 (s, 3H). [M+H]⁺=988.4.

### Example 39: (R)-3-(4-(3-((9-(4-((5-bromo-4-((5-(dimethylphosphoryl)-3-fluoro-2-methylquinolin-6-yl)amino)pyrimidin-2-yl)amino)-2-ethyl-5-methoxyphenyl)-3,9-diazaspiro[5.5]undecan-3-yl)methyl)azetidin-1-yl)-2,6-difluorophenyl)piperidine-2,6-dione

### Step 1: methyl 1-(4-(2,6-bis(benzyloxy)pyridin-3-yl)-3,5-difluorophenyl)azetidine-3-carboxylate

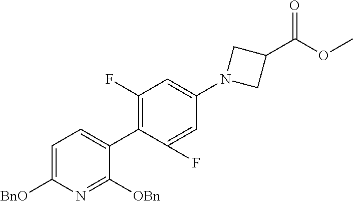 [SMILES: COC(=O)C1CN(c2cc(F)c(-c3ccc(OCc4ccccc4)nc3OCc3ccccc3)c(F)c2)C1]

A mixture of 2,6-bis(benzyloxy)-3-(4-bromo-2,6-difluorophenyl)pyridine (3.00 g, 6.22 mmol), methyl azetidine-3-carboxylate hydrochloride (1.41 g, 9.33 mmol), Cs₂CO₃ (6.06 g, 18.66 mmol) and RuPhos Pd G3 (520.7 mg, 0.622 mmol) in toluene (50 mL) was stirred overnight at 100° C. under nitrogen atmosphere. The resulting mixture was extracted with EtOAc. The combined organic layers were washed with brine, dried over anhydrous Na₂SO₄. After filtration, the filtrate was concentrated under reduced pressure. The residue was purified by silica gel column chromatography, eluted with PE/EtOAc (2:1) to afford the product (1.7 g, 53%). [M+1]⁺=517.1.

### Step 2: (1-(4-(2,6-bis(benzyloxy)pyridin-3-yl)-3,5-difluorophenyl)azetidin-3-yl)methanol

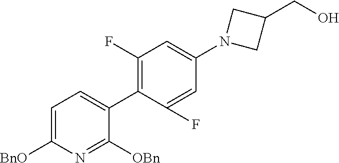 [SMILES: OCC1CN(c2cc(F)c(-c3ccc(OCc4ccccc4)nc3OCc3ccccc3)c(F)c2)C1]

To a stirred mixture of methyl 1-(4-(2,6-bis(benzyloxy)pyridin-3-yl)-3,5-difluorophenyl)azetidine-3-carboxylate (1.7 g, 3.29 mmol) in THF (20 mL) was added LiAlH₄ (1 M in THF, 4.27 mL, 4.27 mmol) dropwise at 0° C. Then the mixture was stirred for 2 hours, the reaction was quenched with water (10 mL) at 0° C. The resulting mixture was extracted with EtOAc (3×50 mL). The combined organic layers were washed with brine, dried over anhydrous Na₂SO₄. After filtration, the filtrate was concentrated under reduced pressure to afford the product (1.4 g, 87%) [M+1]⁺=489.2.

### Step 3: 3-(2,6-difluoro-4-(3-(hydroxymethyl)azetidin-1-yl)phenyl)piperidine-2,6-dione

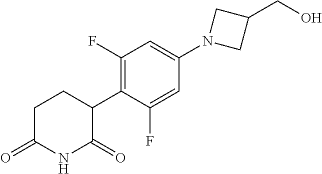 [SMILES: O=C1CCC(c2c(F)cc(N3CC(CO)C3)cc2F)C(=O)N1]

To a solution of (1-(4-(2,6-bis(benzyloxy)pyridin-3-yl)-3,5-difluorophenyl)azetidin-3-yl)methanol (1.40 g, 2.87 mmol) in iPrOH (20 mL) and DCM (20 mL) was added Pd/C (1.0 g, 10% wt) which was stirred at room temperature under hydrogen atmosphere for 48 hours. The resulting mixture was filtered, the filter cake was washed with MeOH (20 mL). The filtrate was concentrated under reduced pressure to afford the product (450 mg, 51%). [M+1]*+=311.3.

### Step 4: 1-(4-(2,6-dioxopiperidin-3-yl)-3,5-difluorophenyl)azetidine-3-carbaldehyde

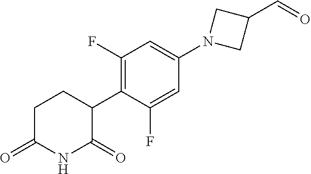 [SMILES: O=CC1CN(c2cc(F)c(C3CCC(=O)NC3=O)c(F)c2)C1]

The titled compound (258 mg, 48%) was prepared in a manner similar to that in Example 7 step 3 from 3-(2,6-difluoro-4-(3-(hydroxymethyl)azetidin-1-yl)phenyl)piperidine-2,6-dione and DMP.

### Step 5: (R)-3-(4-(3-((9-(4-((5-bromo-4-((5-(dimethylphosphoryl)-3-fluoro-2-methylquinolin-6-yl)amino)pyrimidin-2-yl)amino)-2-ethyl-5-methoxyphenyl)-3,9-diazaspiro[5.5]undecan-3-yl)methyl)azetidin-1-yl)-2,6-difluorophenyl)piperidine-2,6-dione

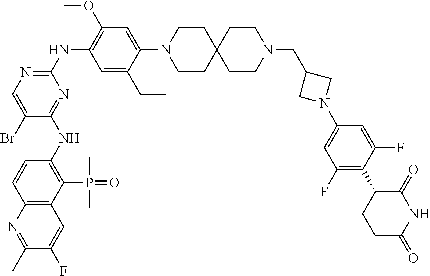 [SMILES: CCc1cc(Nc2ncc(Br)c(Nc3ccc4nc(C)c(F)cc4c3P(C)(C)=O)n2)c(OC)cc1N1CCC2(CCN(CC3CN(c4cc(F)c([C@H]5CCC(=O)NC5=O)c(F)c4)C3)CC2)CC1]

The titled compound (18 mg, 38%) was prepared in a manner similar to that in Example 7 step 8 from (6-((5-bromo-2-((5-ethyl-2-methoxy-4-(3,9-diazaspiro[5.5]undecan-3-yl)phenyl)amino)pyrimidin-4-yl)amino)-3-fluoro-2-methylquinolin-5-yl)dimethylphosphine oxide and 1-(4-(2,6-dioxopiperidin-3-yl)-3,5-difluorophenyl)azetidine-3-carbaldehyde. ¹H NMR (500 MHz, DMSO) δ 11.43 (s, 1H), 10.87 (s, 1H), 9.22 (s, 1H), 8.69 (d, J=11.3 Hz, 1H), 8.28 (s, 2H), 7.97 (d, J=9.0 Hz, 1H), 7.32 (s, 1H), 6.84 (s, 1H), 6.17 (s, 1H), 6.15 (s, 1H), 4.05 (t, J=7.5 Hz, 3H), 3.78 (s, 3H), 3.63 (t, J=6.0 Hz, 2H), 3.46 (s, 2H), 3.32 (d, J=11.1 Hz, 2H), 3.24-3.16 (m, 1H), 3.07 (d, J=11.8 Hz, 2H), 2.84-2.70 (m, 4H), 2.65 (d, J=2.3 Hz, 4H), 2.17 (s, 2H), 2.10-2.06 (m, 1H), 1.97 (s, 2H), 1.97 (s, 3H), 1.94 (s, 3H), 1.94-1.90 (m, 2H), 1.76 (s, 2H), 1.61-1.47 (m, 4H), 0.69 (s, 3H). [M+H]⁺=1002.7

### Example 40: (R)-3-(4-(2-(9-(4-((5-bromo-4-((5-(dimethylphosphoryl)-2-ethylquinazolin-6-yl)amino)pyrimidin-2-yl)amino)-2-ethyl-5-methoxyphenyl)-3,9-diazaspiro[5.5]undecan-3-yl)ethyl)-2,6-difluorophenyl)piperidine-2,6-dione

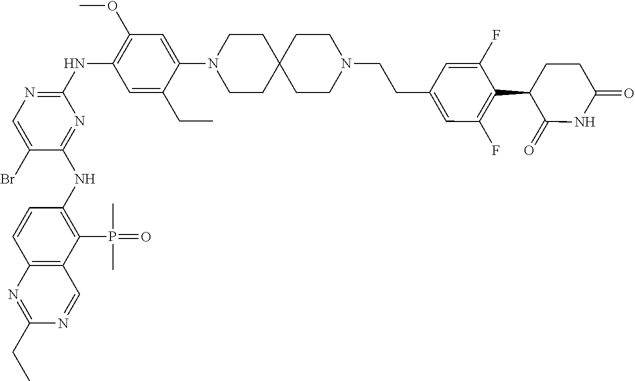 [SMILES: CCc1ncc2c(P(C)(C)=O)c(Nc3nc(Nc4cc(CC)c(N5CCC6(CCN(CCc7cc(F)c([C@H]8CCC(=O)NC8=O)c(F)c7)CC6)CC5)cc4OC)ncc3Br)ccc2n1]

The title compound was prepared in a manner similar to that in Example 1.

¹H NMR (500 MHz, DMSO) δ 11.66 (s, 1H), 10.95 (s, 1H), 9.83 (s, 1H), 8.47 (s, 1H), 8.23 (s, 1H), 8.08 (s, 1H), 7.84 (d, J=9.3 Hz, 1H), 7.25 (s, 1H), 7.03 (d, J=10.2 Hz, 2H), 6.82 (s, 1H), 4.20 (dd, J=12.5, 4.8 Hz, 1H), 3.76 (s, 3H), 3.29 (s, 1H), 3.05 (q, J=7.5 Hz, 2H), 2.85-2.71 (m, 7H), 2.57-2.53 (m, 2H), 2.48-2.42 (m, 4H), 2.33-2.22 (m, 2H), 2.17-2.08 (m, 1H), 2.06-1.97 (m, 7H), 1.55 (s, 8H), 1.38 (t, J=7.6 Hz, 3H), 0.77 (s, 3H); [M+H]⁺=958.6.

### Example 41: (R)-3-(4-(2-(9-(4-((5-chloro-4-((5-(dimethylphosphoryl)-3-fluoro-2-methylquinolin-6-yl)amino)pyrimidin-2-yl)amino)-2-ethyl-5-methoxyphenyl)-3,9-diazaspiro[5.5]undecan-3-yl)ethyl)-2,6-difluorophenyl)piperidine-2,6-dione

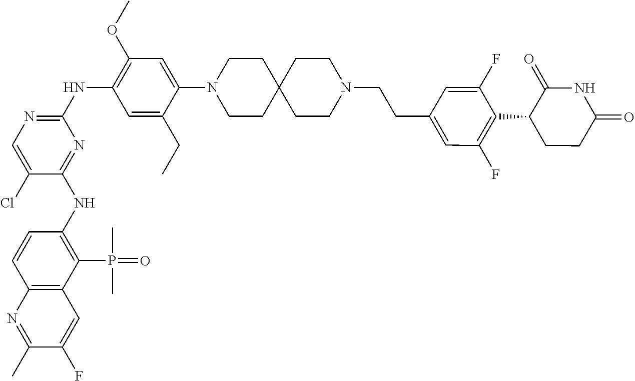 [SMILES: CCc1cc(Nc2ncc(Cl)c(Nc3ccc4nc(C)c(F)cc4c3P(C)(C)=O)n2)c(OC)cc1N1CCC2(CCN(CCc3cc(F)c([C@H]4CCC(=O)NC4=O)c(F)c3)CC2)CC1]

The title compound was prepared in a procedure similar to that in Example 1. ¹H NMR (500 MHz, DMSO) δ 11.68 (s, 1H), 10.95 (s, 1H), 8.53 (d, J=11.0 Hz, 1H), 8.36 (s, 1H), 8.15 (s, 1H), 8.01 (s, 1H), 7.93 (d, J=9.2 Hz, 1H), 7.32 (s, 1H), 7.02 (d, J=10.1 Hz, 2H), 6.81 (s, 1H), 4.23-4.17 (m, 1H), 3.76 (s, 3H), 2.85-2.69 (m, 7H), 2.64 (d, J=2.3 Hz, 3H), 2.58-2.53 (m, 3H), 2.44 (s, 4H), 2.26 (d, J=6.4 Hz, 2H), 2.17-2.08 (m, 1H), 2.02-1.93 (m, 7H), 1.58-1.47 (m, 8H), 0.77 (s, 3H). [M+H]⁺=917.5.

### Example 42: (R)-3-(4-(2-(9-(4-((5-chloro-4-((5-(dimethylphosphoryl)-2-ethylquinazolin-6-yl)amino)pyrimidin-2-yl)amino)-5-methoxy-2-methylphenyl)-3,9-diazaspiro[5.5]undecan-3-yl)ethyl)-2,6-difluorophenyl)piperidine-2,6-dione

### Step 1: (6-((2,5-dichloropyrimidin-4-yl)amino)-2-ethylquinazolin-5-yl)dimethylphosphine oxide

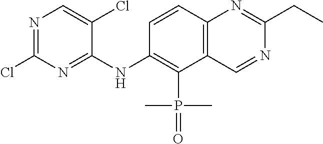 [SMILES: CCc1ncc2c(P(C)(C)=O)c(Nc3nc(Cl)ncc3Cl)ccc2n1]

To a solution of (6-amino-2-ethylquinazolin-5-yl)dimethylphosphine oxide (500 mg, 2.0 mmol) in THF (10 mL) was added 2,4,5-trichloropyrimidine (548.19 g, 3.01 mmol). LiHMDS (1M in THF, 4 mL, 4 mmol) was added to the reaction mixture at 0° C. The mixture was stirred at 20° C. for 3 hrs. The mixture was diluted with water (20 mL), and the layers were separated. The aqueous layer was extracted with DCM (20 mL×3).

The combined organic layers were washed with brine (20 mL×3), dried over Na₂SO₄, filtered and concentrated under reduced pressure. The residue was purified by column chromatography (DCM/MeOH=20/1 to 10/1) to afford the product 600 mg, 75.9%). [M+H]⁺=396.2.

### Step 2: (6-((5-chloro-2-((2-methoxy-5-methyl-4-(3,9-diazaspiro[5.5]undecan-3-yl)phenyl)amino)pyrimidin-4-yl)amino)-2-ethylquinazolin-5-yl)dimethylphosphine oxide

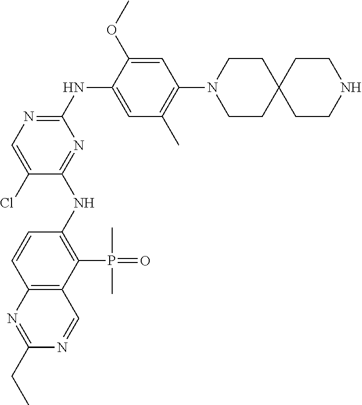 [SMILES: CCc1ncc2c(P(C)(C)=O)c(Nc3nc(Nc4cc(C)c(N5CCC6(CCNCC6)CC5)cc4OC)ncc3Cl)ccc2n1]

To a stirred solution of (6-((2,5-dichloropyrimidin-4-yl)amino)-2-ethylquinazolin-5-yl)dimethylphosphine oxide (600 mg, 1.52 mmol) and tert-butyl 9-(4-amino-5-methoxy-2-methylphenyl)-3,9-diazaspiro[5.5]undecane-3-carboxylate (the intermediate was obtained through the similar way of Example 1 step 7) (594.3 mg, 1.52 mmol) in n-BuOH (10 mL) was added TsOH (784.32 mg, 4.56 mmol). The resulting mixture was stirred at 95° C. for 16 hours. The reaction mixture was concentrated under vacuum before aqueous NaOH (1M, 10 mL) was added into the mixture. Then the mixture was extracted with DCM (3×20 mL). The combined organic layers were washed with brine (3×20 mL), dried over Na₂SO₄, filtered and concentrated under reduced pressure to afford the crude residue, which was purified with silica gel column chromatography (DCM:MeOH=6:1) to give the title product (600 mg, 60.9%). [M+H]⁺=649.3.

### Step 3: (R)-3-(4-(2-(9-(4-((5-chloro-4-((5-(dimethylphosphoryl)-2-ethylquinazolin-6-yl)amino)pyrimidin-2-yl)amino)-5-methoxy-2-methylphenyl)-3,9-diazaspiro[5.5]undecan-3-yl)ethyl)-2,6-difluorophenyl)piperidine-2,6-dione

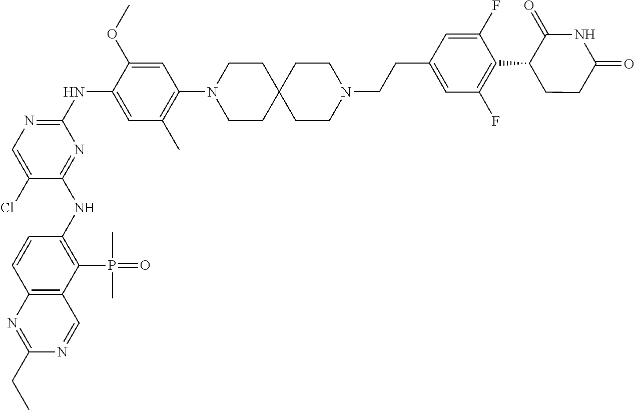 [SMILES: CCc1ncc2c(P(C)(C)=O)c(Nc3nc(Nc4cc(C)c(N5CCC6(CCN(CCc7cc(F)c([C@H]8CCC(=O)NC8=O)c(F)c7)CC6)CC5)cc4OC)ncc3Cl)ccc2n1]

To a solution of (6-((5-chloro-2-((2-methoxy-5-methyl-4-(3,9-diazaspiro[5.5]undecan-3-yl)phenyl)amino)pyrimidin-4-yl)amino)-2-ethylquinazolin-5-yl)dimethylphosphine oxide (50 mg, 0.077 mmol) and (R)-2-(4-(2,6-dioxopiperidin-3-yl)-3,5-difluorophenyl)acetaldehyde (40 mg, 0.15 mmol) in DCM (3 mL) at 20° C. The mixture was stirred at 20° C. for 1 hr. The mixture was diluted with water (20 mL), and the layers were separated. The aqueous layer was extracted with DCM (20 mL×3). The combined organic layers were washed with brine (20 mL×3), dried over Na₂SO₄, filtered and concentrated under reduced pressure. The residue was purified by prep-HPLC (C-18 column chromatography (0.1% FA in water:acetonitrile=90:10˜60:40 gradient elution) to afford the product (24.5 mg, 19.6%). ¹H NMR (500 MHz, DMSO) δ 11.61 (s, 1H), 10.95 (s, 1H), 9.86 (s, 1H), 8.45 (s, 1H), 8.24 (s, 1H), 8.05 (s, 1H), 7.85 (d, J=8.8 Hz, 1H), 7.28 (s, 1H), 7.06 (d, J=10.2 Hz, 2H), 6.75 (s, 1H), 4.21 (dd, J=12.5, 4.7 Hz, 1H), 3.75 (s, 3H), 3.52-3.44 (m, 2H), 3.05 (dd, J=15.1, 7.6 Hz, 2H), 2.94 (d, J=10.2 Hz, 2H), 2.87-2.76 (m, 2H), 2.72-2.63 (m, 3H), 2.29 (d, J=22.7 Hz, 7H), 2.20-2.12 (m, 3H), 2.03 (d, J=13.4 Hz, 7H), 1.84 (s, 2H), 1.55 (d, J=10.0 Hz, 2H), 1.38 (s, 3H), 0.76 (s, 3H). [M+H]⁺=900.5.

### Example 43: (R)-3-(4-((2-(9-(4-((5-bromo-4-((5-(dimethylphosphoryl)-3-fluoro-2-methylquinolin-6-yl)amino)pyrimidin-2-yl)amino)-2-ethyl-5-methoxyphenyl)-3,9-diazaspiro[5.5]undecan-3-yl)ethyl)amino)-2,6-difluorophenyl)piperidine-2,6-dione

### Step 1: 2-((4-(2,6-bis(benzyloxy)pyridin-3-yl)-3,5-difluorophenyl)amino)ethan-1-ol

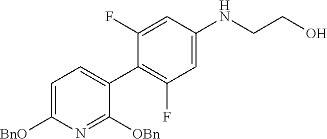 [SMILES: OCCNc1cc(F)c(-c2ccc(OCc3ccccc3)nc2OCc2ccccc2)c(F)c1]

To the solution of 2,6-bis(benzyloxy)-3-(4-bromo-2,6-difluorophenyl)pyridine (1.92 g, 4 mmol), 2-aminoethan-1-ol (488 mg, 8 mmol) and K₃PO₄ (1.7 g, 8 mmol) in 15 mL DMSO, CuI (76 mg, 0.4 mmol) and L-Proline (92 mg, 0.8 mmol) was added under N₂ protected. The mixture was stirred at 100° C. for 16 hours.

After LCMS shown the reaction was completed. The mixture was diluted with EtOAc and filtered. The filtrate was washed with saturated NaCl aqueous and concentrated in vacuum. The crude was purified with silica column chromatography (EA:PE=0-50%) to afford the title compound (1.5 g, 81.2% yield). [M+H]⁺=463.6.

### Step 2: 3-(2,6-difluoro-4-((2-hydroxyethyl)amino)phenyl)piperidine-2,6-dione

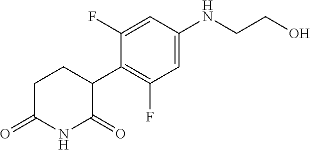 [SMILES: O=C1CCC(c2c(F)cc(NCCO)cc2F)C(=O)N1]

To the solution of 2-((4-(2,6-bis(benzyloxy)pyridin-3-yl)-3,5-difluorophenyl)amino)ethan-1-ol (1.5 g, 3.2 mmol) in 5 mL DCM and 40 mL IPA, Pd/C (1.5 g, 10% w.t.) was added. The mixture was stirred at 45° C. for 16 hours at H₂ atmosphere. After LCMS shown the reaction was completed. The mixture was filtered by celite. The filtrate was concentrated in vacuum to afford the title compound (500 mg, 55% yield). [M+H]⁺=285.6.

### Step 3:2-((4-(26-dioxopiperidin-3-yl)-3,5-difluorophenyl)amino)ethyl methanesulfonate

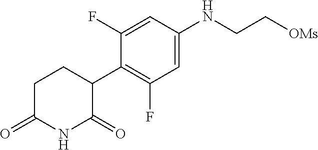 [SMILES: CS(=O)(=O)OCCNc1cc(F)c(C2CCC(=O)NC2=O)c(F)c1]

To the solution of 3-(2,6-difluoro-4-((2-hydroxyethyl)amino)phenyl)piperidine-2,6-dione (50 mg, 0.18 mmol) and Et₃N (40 mg, 0.4 mmol) in 1.5 mL DCM, MsCl (23 mg, 0.2 mmol) was added. The mixture was stirred at room temperature for 1 hour. After LCMS shown the reaction was completed. The mixture was concentrated in vacuum and purified by prep-TLC (DCM:MeOH=15:1) to afford the title compound (52 mg, 80% yield). [M+H]⁺=363.6.

### Step 4: (R)-3-(4-((2-(9-(4-((5-bromo-4-((5-(dimethylphosphoryl)-3-fluoro-2-methylquinolin-6-yl)amino)pyrimidin-2-yl)amino)-2-ethyl-5-methoxyphenyl)-3,9-diazaspiro[5.5]undecan-3-yl)ethyl)amino)-2,6-difluorophenyl)piperidine-2,6-dione

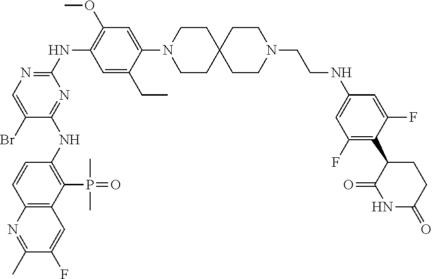 [SMILES: CCc1cc(Nc2ncc(Br)c(Nc3ccc4nc(C)c(F)cc4c3P(C)(C)=O)n2)c(OC)cc1N1CCC2(CCN(CCNc3cc(F)c([C@H]4CCC(=O)NC4=O)c(F)c3)CC2)CC1]

To the solution of (6-((5-bromo-2-((5-ethyl-2-methoxy-4-(3,9-diazaspiro[5.5]undecan-3-yl)phenyl)amino)pyrimidin-4-yl)amino)-3-fluoro-2-methylquinolin-5-yl)dimethylphosphine oxide (100 mg, 0.14 mmol), 2-((4-(2,6-dioxopiperidin-3-yl)-3,5-difluorophenyl)amino)ethyl methanesulfonate (52 mg, 0.14 mmol), KI (33 mg, 0.2 mmol) and DIEA (40 mg, 0.31 mmol) in 3 mL ACN and 0.5 mL DMSO. The mixture was stirred at 80° C. for 16 hours. After LCMS shown the reaction was completed. The mixture was concentrated in vacuum. The mixture was washed with water and extracted by DCM. The organic layer was concentrated in vacuum and purified by prep-TLC (DCM:MeOH=11:1) to afford the crude product. The crude was separated by SFC (IF (2 cm×25 cm, Sum), MtBE (0.1% DEA):(MeOH:DCM:=1:1)=50:50, 100 bar, 20 ml/min) and the title compound corresponded to peak A @ 1.202 min/254 nm (29.78 mg, 22% yield).

¹H NMR (500 MHz, DMSO) δ 11.34 (s, 1H), 10.83 (s, 1H), 8.60 (d, J=12.3 Hz, 1H), 8.27-8.15 (m, 2H), 8.02-7.90 (m, 2H), 7.30 (s, 1H), 6.79 (s, 1H), 6.27 (d, J=12.5 Hz, 2H), 6.07 (s, 1H), 3.98 (dd, J=12.0, 4.3 Hz, 1H), 3.76 (s, 3H), 3.12 (s, 2H), 2.83-2.69 (m, 5H), 2.64 (s, 3H), 2.49-2.32 (m, 7H), 2.28-2.13 (m, 2H), 2.09-2.01 (m, 1H), 1.99-1.84 (m, 7H), 1.55 (s, 8H), 0.72 (s, 3H); [M+H]⁺=976.6.

### Example 44: (R)-3-(4-((2-(9-(4-((5-bromo-4-((5-(dimethylphosphoryl)-3-fluoro-2-methylquinolin-6-yl)amino)pyrimidin-2-yl)amino)-2-ethyl-5-methoxyphenyl)-3,9-diazaspiro[5.5]undecan-3-yl)ethyl)(methyl)amino)-2,6-difluorophenyl)piperidine-2,6-dione

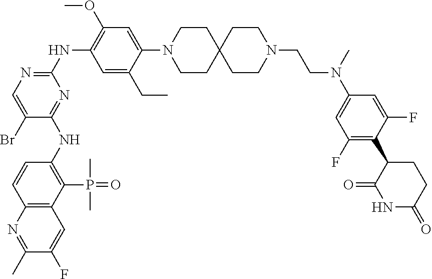 [SMILES: CCc1cc(Nc2ncc(Br)c(Nc3ccc4nc(C)c(F)cc4c3P(C)(C)=O)n2)c(OC)cc1N1CCC2(CCN(CCN(C)c3cc(F)c([C@H]4CCC(=O)NC4=O)c(F)c3)CC2)CC1]

The title compound was prepared in a manner similar to that in Example 43.

¹H NMR (500 MHz, DMSO) δ 11.34 (s, 1H), 10.84 (s, 1H), 8.59 (d, J=12.1 Hz, 1H), 8.26-8.16 (m, 2H), 8.01-7.89 (m, 2H), 7.29 (s, 1H), 6.79 (s, 1H), 6.34 (d, J=12.9 Hz, 2H), 4.01 (dd, J=12.0, 3.5 Hz, 1H), 3.75 (s, 3H), 3.44 (s, 2H), 2.91 (s, 3H), 2.82-2.67 (m, 5H), 2.63 (s, 3H), 2.47-2.36 (m, 7H), 2.24-2.12 (m, 2H), 2.11-2.04 (m, 1H), 2.01-1.80 (m, 7H), 1.52 (s, 8H), 0.72 (s, 3H); [M+H]⁺=990.6.

### Example 45: 4-(4-((9-(4-((5-bromo-4-((5-(dimethylphosphoryl)-3-fluoro-2-methylquinolin-6-yl)amino)pyrimidin-2-yl)amino)-2-ethyl-5-methoxyphenyl)-3,9-diazaspiro[5.5]undecan-3-yl)methyl)piperidin-1-yl)-2-(2,6-dioxopiperidin-3-yl)isoindoline-1,3-dione

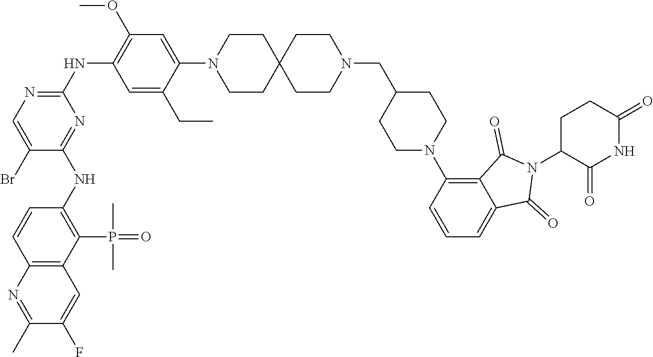 [SMILES: CCc1cc(Nc2ncc(Br)c(Nc3ccc4nc(C)c(F)cc4c3P(C)(C)=O)n2)c(OC)cc1N1CCC2(CCN(CC3CCN(c4cccc5c4C(=O)N(C4CCC(=O)NC4=O)C5=O)CC3)CC2)CC1]

The title compound was prepared in a manner similar to that in Example 35.

¹H NMR (500 MHz, DMSO) δ 11.34 (s, 1H), 11.09 (s, 1H), 8.61 (d, J=12.6 Hz, 1H), 8.28-8.14 (m, 2H), 8.03-7.90 (m, 2H), 7.68 (t, J=7.4 Hz, 1H), 7.41-7.22 (m, 3H), 6.78 (s, 1H), 5.09 (dd, J=12.5, 5.2 Hz, 1H), 3.83-3.61 (m, 6H), 2.91-2.83 (m, 3H), 2.72 (s, 3H), 2.66-2.57 (m, 5H), 2.48-2.37 (m, 3H), 2.31-2.17 (m, 3H), 2.06-1.93 (m, 9H), 1.86-1.66 (m, 4H), 1.55 (s, 7H), 1.37-1.22 (m, 2H), 0.72 (s, 3H);
[M+H]⁺=1063.6.

### Example 46: 5-(4-((9-(4-((5-bromo-4-((5-(dimethylphosphoryl)-3-fluoro-2-methylquinolin-6-yl)amino)pyrimidin-2-yl)amino)-2-ethyl-5-methoxyphenyl)-3,9-diazaspiro [5.5]undecan-3-yl)methyl)piperidin-1-yl)-2-(2,6-dioxopiperidin-3-yl)isoindoline-1,3-dione

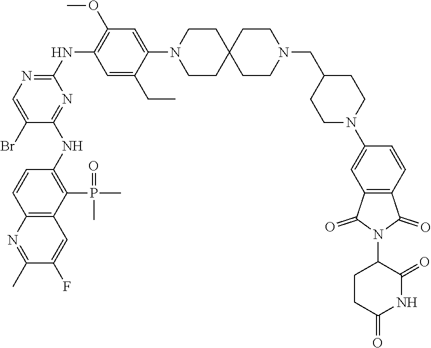 [SMILES: CCc1cc(Nc2ncc(Br)c(Nc3ccc4nc(C)c(F)cc4c3P(C)(C)=O)n2)c(OC)cc1N1CCC2(CCN(CC3CCN(c4ccc5c(c4)C(=O)N(C4CCC(=O)NC4=O)C5=O)CC3)CC2)CC1]

The title compound was prepared in a manner similar to that in Example 35.

¹H NMR (500 MHz, DMSO) δ 11.34 (s, 1H), 11.07 (s, 1H), 8.60 (d, J=11.7 Hz, 1H), 8.22 (s, 2H), 8.01-7.84 (m, 2H), 7.65 (d, J=8.4 Hz, 1H), 7.30 (s, 2H), 7.22 (s, 1H), 6.78 (s, 1H), 5.06 (dd, J=12.7, 5.3 Hz, 1H), 4.04 (d, J=12.3 Hz, 2H), 3.75 (s, 3H), 2.92-2.89 (m, 3H), 2.71 (s, 4H), 2.65-2.52 (m, 5H), 2.36 (s, 4H), 2.17 (s, 4H), 2.01-1.98 (m, 2H), 1.95-1.91 (m, 6H), 1.89-1.75 (m, 3H), 1.53 (s, 7H), 1.21-1.04 (m, 2H), 0.72 (s, 3H); [M+H]⁺=1063.5.

### Example 47: 5-(4-((9-(4-((5-bromo-4-((5-(dimethylphosphoryl)-3-fluoro-2-methylquinolin-6-yl)amino)pyrimidin-2-yl)amino)-2-ethyl-5-methoxyphenyl)-3,9-diazaspiro[5.5]undecan-3-yl)methyl)piperidin-1-yl)-2-(2,6-dioxopiperidin-3-yl)-6-fluoroisoindoline-1,3-dione

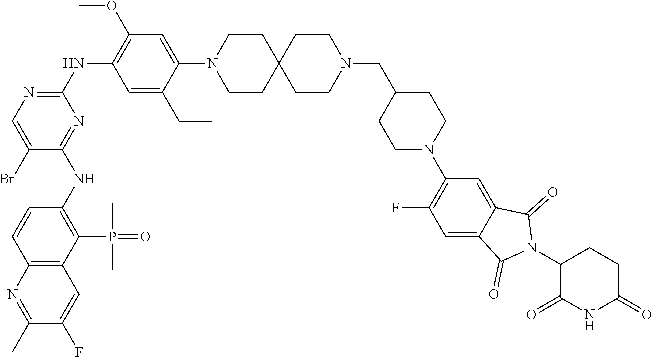 [SMILES: CCc1cc(Nc2ncc(Br)c(Nc3ccc4nc(C)c(F)cc4c3P(C)(C)=O)n2)c(OC)cc1N1CCC2(CCN(CC3CCN(c4cc5c(cc4F)C(=O)N(C4CCC(=O)NC4=O)C5=O)CC3)CC2)CC1]

The title compound was prepared in a manner similar to that in Example 35.

¹H NMR (500 MHz, DMSO) δ 11.27 (s, 1H), 11.11 (s, 1H), 8.74 (s, 1H), 8.66 (d, J=9.7 Hz, 1H), 8.19 (s, 1H), 7.95 (d, J=4.5 Hz, 2H), 7.74 (d, J=11.7 Hz, 1H), 7.48 (d, J=7.3 Hz, 1H), 7.33 (s, 1H), 6.80 (s, 1H), 5.11 (dd, J=12.6, 4.8 Hz, 1H), 3.77 (s, 3H), 3.66 (d, J=9.3 Hz, 2H), 3.46-3.37 (m, 2H), 3.09 (s, 4H), 2.97-2.85 (m, 4H), 2.74 (s, 3H), 2.66-2.60 (m, 5H), 2.25-2.15 (m, 2H), 2.08-2.00 (m, 2H), 1.98-1.93 (m, 8H), 1.89-1.85 (m, 2H), 1.78-1.72 (m, 2H), 1.67-1.59 (m, 2H), 1.55-1.47 (m, 2H), 1.45-1.36 (m, 2H), 0.70 (s, 3H); [M+H]⁺=1081.7.

### Example 48: 3-(5-(4-((9-(4-((5-bromo-4-((5-(dimethylphosphoryl)-3-fluoro-2-methylquinolin-6-yl)amino)pyrimidin-2-yl)amino)-2-ethyl-5-methoxyphenyl)-3,9-diazaspiro[5.5]undecan-3-yl)methyl)piperidin-1-yl)-1-oxoisoindolin-2-yl)piperidine-2,6-dione

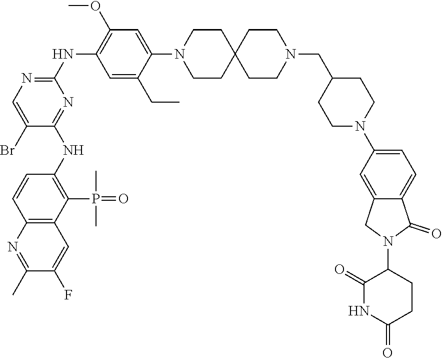 [SMILES: CCc1cc(Nc2ncc(Br)c(Nc3ccc4nc(C)c(F)cc4c3P(C)(C)=O)n2)c(OC)cc1N1CCC2(CCN(CC3CCN(c4ccc5c(c4)CN(C4CCC(=O)NC4=O)C5=O)CC3)CC2)CC1]

The title compound was prepared in a manner similar to that in Example 35.

¹H NMR (500 MHz, DMSO) δ 11.34 (s, 1H), 10.94 (s, 1H), 8.60 (d, J=12.1 Hz, 1H), 8.26-8.14 (m, 2H), 8.01-7.89 (m, 2H), 7.50 (d, J=8.4 Hz, 1H), 7.30 (s, 1H), 7.04 (d, J=8.9 Hz, 2H), 6.78 (s, 1H), 5.04 (dd, J=13.3, 5.0 Hz, 1H), 4.38-4.16 (m, 2H), 3.87 (d, J=11.1 Hz, 2H), 3.76 (s, 3H), 2.98-2.76 (m, 4H), 2.71 (s, 3H), 2.67-2.54 (m, 6H), 2.42-2.34 (m, 4H), 2.26-2.08 (m, 4H), 2.00-1.91 (m, 7H), 1.80-1.74 (m, 2H), 1.54 (s, 8H), 1.26-1.09 (m, 2H), 0.72 (s, 3H); [M+H]⁺=1049.9.

### Example 49: 5-((S)-3-((9-(4-((5-bromo-4-((5-(dimethylphosphoryl)-3-fluoro-2-methylquinolin-6-yl)amino)pyrimidin-2-yl)amino)-2-ethyl-5-methoxyphenyl)-3,9-diazaspiro[5.5]undecan-3-yl)methyl)pyrrolidin-1-yl)-2-(2,6-dioxopiperidin-3-yl)isoindoline-1,3-dione

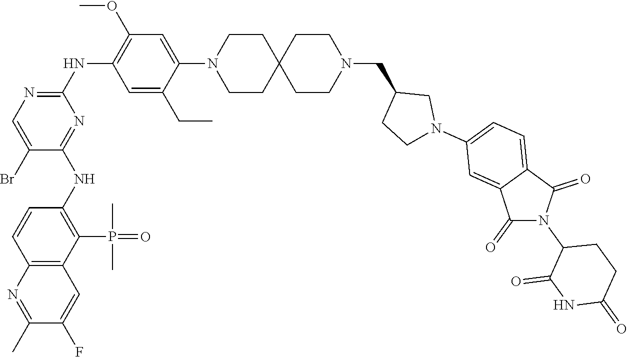 [SMILES: CCc1cc(Nc2ncc(Br)c(Nc3ccc4nc(C)c(F)cc4c3P(C)(C)=O)n2)c(OC)cc1N1CCC2(CCN(C[C@@H]3CCN(c4ccc5c(c4)C(=O)N(C4CCC(=O)NC4=O)C5=O)C3)CC2)CC1]

The title compound was prepared in a manner similar to that in Example 35.

¹H NMR (500 MHz, DMSO) δ 11.34 (s, 1H), 11.06 (s, 1H), 8.60 (d, J=11.8 Hz, 1H), 8.21 (d, J=10.9 Hz, 2H), 7.94 (d, J=13.8 Hz, 2H), 7.64 (d, J=8.5 Hz, 1H), 7.31 (s, 1H), 6.90 (s, 1H), 6.85-6.75 (m, 2H), 5.05 (dd, J=12.8, 5.2 Hz, 1H), 3.76 (s, 3H), 3.59-3.48 (m, 2H), 3.42-3.38 (m, 1H), 3.17-3.10 (m, 1H), 2.92-2.84 (m, 1H), 2.72 (s, 4H), 2.67-2.52 (m, 8H), 2.42-2.32 (m, 4H), 2.20 (s, 1H), 2.15-2.10 (m, 1H), 2.02-1.92 (m, 8H), 1.78-1.72 (m, 1H), 1.55 (s, 8H), 0.72 (s, 3H); [M+H]⁺=1049.8.

### Example 51: 3-(4-(4-(9-(4-((5-bromo-4-((5-(dimethylphosphoryl)-3-fluoro-2-methylquinolin-6-yl)amino)pyrimidin-2-yl)amino)-2-ethyl-5-methoxyphenyl)-3,9-diazaspiro[5.5]undecan-3-yl)piperidin-1-yl)-3-methyl-2-oxo-2,3-dihydro-1H-benzo[d]imidazol-1-yl)piperidine-2,6-dione

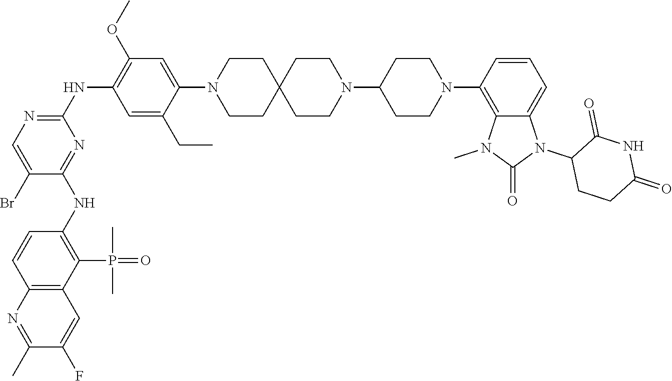 [SMILES: CCc1cc(Nc2ncc(Br)c(Nc3ccc4nc(C)c(F)cc4c3P(C)(C)=O)n2)c(OC)cc1N1CCC2(CC1)CCN(C1CCN(c3cccc4c3n(C)c(=O)n4C3CCC(=O)NC3=O)CC1)CC2]

The title compound was prepared in a manner similar to that in Example 21.

¹H NMR (500 MHz, DMSO) δ 11.31 (s, 1H), 11.09 (s, 1H), 8.64 (d, J=12.2 Hz, 1H), 8.23 (s, 1H), 8.20 (s, 1H), 7.96 (s, 1H), 7.94 (s, 1H), 7.33 (s, 1H), 6.99 (t, J=7.9 Hz, 1H), 6.92 (d, J=8.6 Hz, 1H), 6.89 (s, 1H), 6.80 (s, 1H), 5.36 (dd, J=12.7, 5.2 Hz, 1H), 3.77 (s, 3H), 3.64 (s, 3H), 3.22 (d, J=8.2 Hz, 2H), 2.95-2.84 (m, 2H), 2.81-2.67 (m, 8H), 2.65 (d, J=2.1 Hz, 4H), 2.60 (s, 1H), 2.19 (s, 2H), 2.10 (s, 2H), 2.03-1.90 (m, 9H), 1.85 (s, 2H), 1.60 (s, 8H), 0.71 (s, 3H). [M+H]⁺=1050.7

### Example 52: 3-(4-(4-(9-(4-((5-bromo-4-((5-(dimethylphosphoryl)-3-fluoro-2-methylquinolin-6-yl)amino)pyrimidin-2-yl)amino)-2-ethyl-5-methoxyphenyl)-3,9-diazaspiro[5.5]undecan-3-yl)piperidin-1-yl)-3,3-dimethyl-2-oxoindolin-1-yl)piperidine-2,6-dione

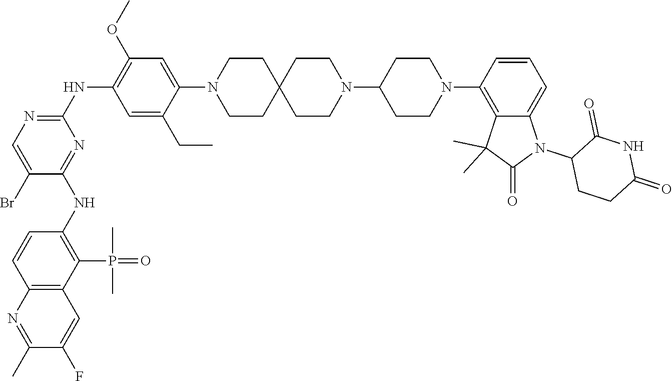 [SMILES: CCc1cc(Nc2ncc(Br)c(Nc3ccc4nc(C)c(F)cc4c3P(C)(C)=O)n2)c(OC)cc1N1CCC2(CC1)CCN(C1CCN(c3cccc4c3C(C)(C)C(=O)N4C3CCC(=O)NC3=O)CC1)CC2]

The title compound was prepared in a manner similar to that in Example 28.

¹H NMR (500 MHz, DMSO) δ 11.31 (s, 1H), 11.09 (s, 1H), 8.64 (d, J=12.2 Hz, 1H), 8.23 (s, 1H), 8.20 (s, 1H), 7.96 (s, 1H), 7.94 (s, 1H), 7.33 (s, 1H), 6.99 (t, J=7.9 Hz, 1H), 6.92 (d, J=8.6 Hz, 1H), 6.89 (s, 1H), 6.80 (s, 1H), 5.36 (dd, J=12.7, 5.2 Hz, 1H), 3.64 (s, 3H), 3.22 (d, J=8.2 Hz, 2H), 2.95-2.84 (m, 2H), 2.81-2.67 (m, 8H), 2.65 (d, J=2.1 Hz, 4H), 2.60 (s, 1H), 2.19 (m, 2H), 2.10 (m, 2H), 2.03-1.98 (m, 2H) 1.97 (s, 3H), 1.94 (s, 3H), 1.94-1.90 (m, 1H), 1.85 (s, 2H), 1.75 (s, 6H), 1.60 (m, 8H), 0.71 (s, 3H). [M+H]+=1063.8

### Example 54: (R)-3-(4-(4-(4-(1-(4-((5-chloro-4-((5-(dimethylphosphoryl)-3-fluoro-2-methylquinolin-6-yl)amino)pyrimidin-2-yl)amino)-5-ethoxy-2-ethylphenyl)piperidin-4-yl)piperazin-1-yl)piperidin-1-yl)-2,6-difluorophenyl)piperidine-2,6-dione

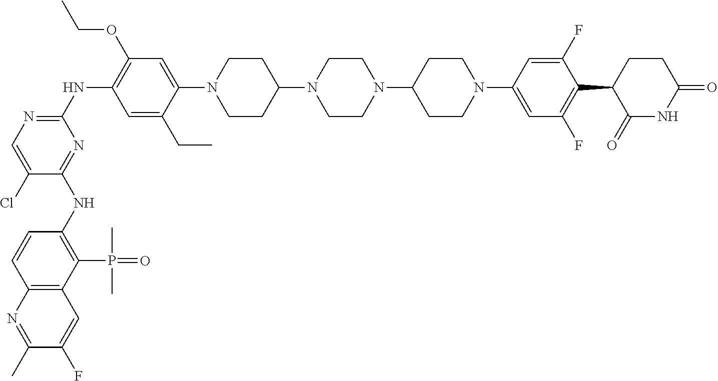 [SMILES: CCOc1cc(N2CCC(N3CCN(C4CCN(c5cc(F)c([C@H]6CCC(=O)NC6=O)c(F)c5)CC4)CC3)CC2)c(CC)cc1Nc1ncc(Cl)c(Nc2ccc3nc(C)c(F)cc3c2P(C)(C)=O)n1]

The title compound was prepared in a manner similar to that in Example 1.

¹H NMR (500 MHz, DMSO) δ 11.56 (s, 1H), 10.87 (s, 1H), 8.61 (d, J=11.7 Hz, 1H), 8.31 (s, 1H), 8.16 (s, 1H), 7.95 (d, J=9.2 Hz, 1H), 7.90 (s, 1H), 7.38 (s, 1H), 6.70 (s, 1H), 6.62 (d, J=12.8 Hz, 2H), 4.07-3.98 (m, 3H), 3.78 (d, J=11.5 Hz, 2H), 3.31-3.26 (m, 1H), 2.94-2.86 (m, 2H), 2.82-2.71 (m, 3H), 2.67-2.57 (m, 6H), 2.49-2.28 (m, 8H), 2.27-2.18 (m, 3H), 2.12-2.05 (m, 1H), 1.99-1.91 (m, 7H), 1.86-1.77 (m, 4H), 1.55-1.39 (m, 4H), 1.26 (t, J=6.7 Hz, 3H), 0.71 (s, 3H); [M+H]⁺=1001.5.

### Example 55: (R)-3-(4-(1-((1-(1-(4-((5-bromo-4-((5-(dimethylphosphoryl)-2-ethylquinazolin-6-yl)amino)pyrimidin-2-yl)amino)-2-ethyl-5-methoxyphenyl)piperidin-4-yl)azetidin-3-yl)methyl)piperidin-4-yl)-2,6-difluorophenyl)piperidine-2,6-dione

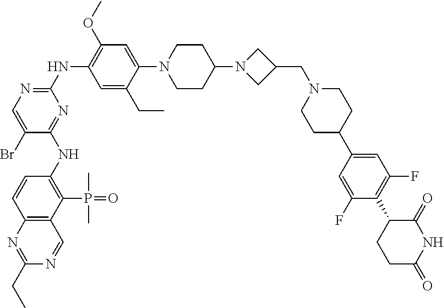 [SMILES: CCc1ncc2c(P(C)(C)=O)c(Nc3nc(Nc4cc(CC)c(N5CCC(N6CC(CN7CCC(c8cc(F)c([C@H]9CCC(=O)NC9=O)c(F)c8)CC7)C6)CC5)cc4OC)ncc3Br)ccc2n1]

The title compound was prepared in a manner similar to that in Example 30.

¹H NMR (500 MHz, DMSO) δ 11.59 (s, 1H), 10.95 (s, 1H), 9.87 (s, 1H), 8.44 (s, 1H), 8.22 (s, 1H), 8.03 (s, 1H), 7.85 (d, J=9.1 Hz, 1H), 7.27 (s, 1H), 7.03 (s, 1H), 7.01 (s, 1H), 6.73 (s, 1H), 4.35 (s, 2H), 4.19 (dd, J=12.4, 4.7 Hz, 1H), 3.75 (s, 3H), 3.46-3.41 (m, 3H), 3.05 (dd, J=14.9, 7.4 Hz, 2H), 2.88 (d, J=7.2 Hz, 2H), 2.83-2.72 (m, 3H), 2.64-2.52 (m, 2H), 2.27-2.16 (m, 2H), 2.14-2.08 (m, 2H), 2.04 (s, 3H), 2.01 (s, 3H), 1.97 (d, J=11.0 Hz, 2H), 1.72 (t, J=11.41 Hz, 4H), 1.66-1.57 (m, 2H), 1.38 (t, J=7.5 Hz, 3H), 1.29 (d, J=9.6 Hz, 2H), 1.06 (t, J=6.9 Hz, 5H), 0.74 (s, 3H). [M+H]⁺=1013.7

### Example 56: (R)-3-(4-(1-((1-(1-(4-((5-bromo-4-((5-(dimethylphosphoryl)-3-fluoro-2-methylquinolin-6-yl)amino)pyrimidin-2-yl)amino)-2-ethyl-5-methoxyphenyl)piperidin-4-yl)azetidin-3-yl)methyl)piperidin-4-yl)-2,6-difluorophenyl)piperidine-2,6-dione

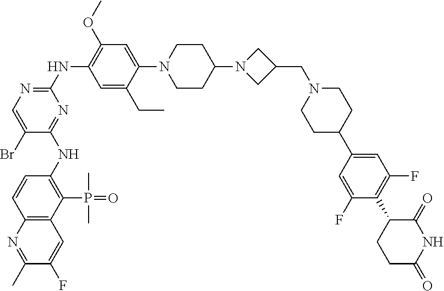 [SMILES: CCc1cc(Nc2ncc(Br)c(Nc3ccc4nc(C)c(F)cc4c3P(C)(C)=O)n2)c(OC)cc1N1CCC(N2CC(CN3CCC(c4cc(F)c([C@H]5CCC(=O)NC5=O)c(F)c4)CC3)C2)CC1]

The title compound was prepared in a manner similar to that in Example 30.

¹H NMR (500 MHz, DMSO) δ 11.30 (s, 1H), 10.94 (s, 1H), 8.63 (d, J=10.6 Hz, 1H), 8.22 (s, 1H), 8.20-8.11 (m, 1H), 7.95 (s, 1H), 7.93 (s, 1H), 7.32 (s, 1H), 7.03 (s, 1H), 7.01 (s, 1H), 6.70 (s, 1H), 4.19 (dd, J=12.5, 4.7 Hz, 1H), 3.75 (s, 3H), 3.35 (s, 3H), 2.91-2.70 (m, 6H), 2.64 (d, J=2.1 Hz, 3H), 2.57 (dd, J=19.7, 9.0 Hz, 4H), 2.48 (s, 2H), 2.13 (dd, J=21.8, 8.3 Hz, 4H), 1.98 (d, J=11.4 Hz, 4H), 1.96 (s, 3H), 1.93 (s, 3H), 1.72 (t, J=15.1 Hz, 4H), 1.61 (d, J=11.4 Hz, 2H), 1.33-1.20 (m, 3H), 0.70 (s, 3H). [M+H]⁺=1016.7

### Example 57: (R)-3-(4-(7-(1-(4-((5-chloro-4-((5-(dimethylphosphoryl)-3-fluoro-2-methylquinolin-6-yl)amino)pyrimidin-2-yl)amino)-5-ethoxy-2-ethylphenyl)piperidin-4-yl)-2,7-diazaspiro[3.5]nonan-2-yl)-2,6-difluorophenyl)piperidine-2,6-dione

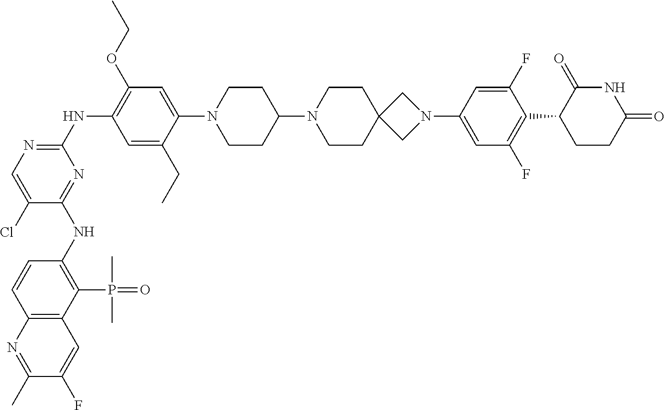 [SMILES: CCOc1cc(N2CCC(N3CCC4(CC3)CN(c3cc(F)c([C@H]5CCC(=O)NC5=O)c(F)c3)C4)CC2)c(CC)cc1Nc1ncc(Cl)c(Nc2ccc3nc(C)c(F)cc3c2P(C)(C)=O)n1]

The title compound was prepared in a procedure similar to that in Example 9. H NMR (500 MHz, DMSO) δ_{H} 11.54 (s, 1H), 10.86 (s, 1H), 8.62 (d, J=12.2 Hz, 1H), 8.31 (s, 1H), 8.17 (s, 1H), 7.95 (d, J=9.2 Hz, 1H), 7.89 (s, 1H), 7.40 (s, 1H), 6.69 (s, 1H), 6.10 (d, J=11.3 Hz, 2H), 4.07-3.96 (m, 3H), 3.55 (s, 4H), 2.91 (d, J=9.7 Hz, 2H), 2.78 (s, 1H), 2.69-2.57 (m, 6H), 2.48-2.35 (m, 4H), 2.20 (d, J=5.9 Hz, 2H), 2.06 (s, 1H), 2.02-1.90 (m, 8H), 1.81-1.70 (m, 6H), 1.57 (d, J=10.1 Hz, 2H), 1.26 (t, J=6.9 Hz, 3H), 0.70 (s, 3H). [M+H]⁺=958.5.

### Example 58: (R)-3-(4-(2-(1-(4-((5-bromo-4-((5-(dimethylphosphoryl)-2-ethylquinolin-6-yl)amino)pyrimidin-2-yl)amino)-2-ethyl-5-methoxyphenyl)piperidin-4-yl)-2,7-diazaspiro[3.5]nonan-7-yl)-2,6-difluorophenyl)piperidine-2,6-dione

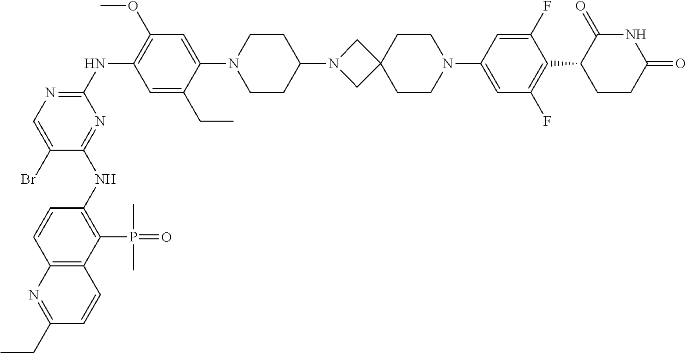 [SMILES: CCc1ccc2c(P(C)(C)=O)c(Nc3nc(Nc4cc(CC)c(N5CCC(N6CC7(CCN(c8cc(F)c([C@H]9CCC(=O)NC9=O)c(F)c8)CC7)C6)CC5)cc4OC)ncc3Br)ccc2n1]

The title compound was prepared in a procedure similar to that in Example 9. ¹H NMR (500 MHz, DMSO) δ 11.79 (s, 1H), 10.87 (s, 1H), 8.66 (s, 1H), 8.53 (s, 1H), 8.25 (s, 1H), 8.07 (s, 1H), 7.93 (s, 1H), 7.49 (s, 1H), 7.40 (s, 1H), 6.75 (s, 1H), 6.68 (d, J=12.7 Hz, 2H), 4.05 (dd, J=12.3, 4.6 Hz, 1H), 4.00-3.90 (m, 4H), 3.78 (s, 3H), 3.65-3.56 (m, 3H), 3.30 (s, 3H), 3.20 (s, 2H), 3.15-3.11 (m, 2H), 3.02 (d, J=9.5 Hz, 2H), 2.96 (d, J=7.2 Hz, 2H), 2.78 (t, J=12.6 Hz, 1H), 2.68-2.62 (m, 2H), 2.24 (s, 2H), 2.08 (d, J=9.4 Hz, 2H), 2.00 (s, 3H), 1.98 (s, 3H), 1.80 (s, 2H), 1.60 (s, 2H), 1.33 (t, J=7.6 Hz, 3H), 0.74 (s, 3H). [M+H]⁺=984.7.

### Example 59: (R)-3-(4-(2-(1-(4-((5-chloro-4-((5-(dimethylphosphoryl)-3-fluoro-2-methylquinolin-6-yl)amino)pyrimidin-2-yl)amino)-5-ethoxy-2-ethylphenyl)piperidin-4-yl)-2,7-diazaspiro[3.5]nonan-7-yl)-2,6-difluorophenyl)piperidine-2,6-dione

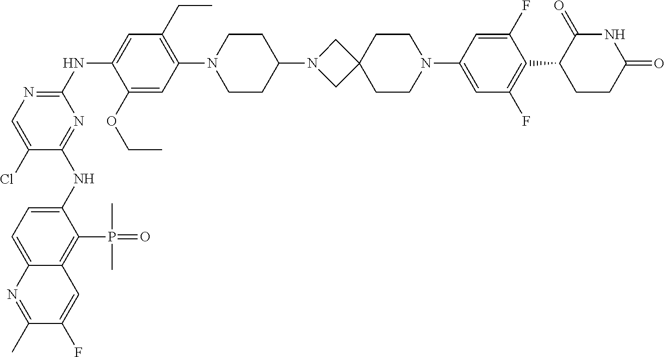 [SMILES: CCOc1cc(N2CCC(N3CC4(CCN(c5cc(F)c([C@H]6CCC(=O)NC6=O)c(F)c5)CC4)C3)CC2)c(CC)cc1Nc1ncc(Cl)c(Nc2ccc3nc(C)c(F)cc3c2P(C)(C)=O)n1]

The title compound was prepared in a procedure similar to that in Example 9. ¹H NMR (500 MHz, DMSO) δ 11.55 (s, 1H), 10.86 (s, 1H), 8.62 (d, J=12.5 Hz, 1H), 8.31 (s, 1H), 8.17 (s, 1H), 7.95 (d, J=9.1 Hz, 1H), 7.89 (s, 1H), 7.38 (s, 1H), 6.69 (s, 1H), 6.63 (d, J=12.9 Hz, 2H), 4.06-3.97 (m, 3H), 3.19 (s, 4H), 2.95 (s, 3H), 2.84 (s, 2H), 2.77 (d, J=12.4 Hz, 1H), 2.64 (s, 3H), 2.58 (t, J=9.9 Hz, 2H), 2.17 (s, 2H), 2.09 (d, J=11.4 Hz, 1H), 1.96 (d, J=13.3 Hz, 7H), 1.72 (s, 6H), 1.57 (s, 1H), 1.34-1.24 (m, 6H), 0.94 (t, J=7.4 Hz, 1H), 0.71 (s, 3H). [M+H]⁺=958.7.

### Example 60: (R)-3-(4-(6-(1-(4-((5-bromo-4-((5-(dimethylphosphoryl)-3-fluoro-2-methylquinolin-6-yl)amino)pyrimidin-2-yl)amino)-2-ethyl-5-methoxyphenyl)piperidin-4-yl)-26-diazaspiro[3.3]heptan-2-yl)-2,6-difluorophenyl)piperidine-2,6-dione

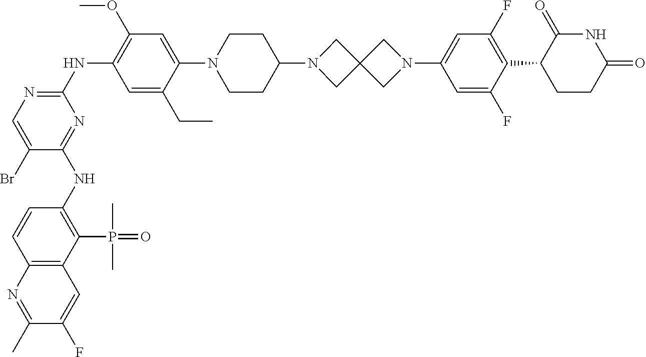 [SMILES: CCc1cc(Nc2ncc(Br)c(Nc3ccc4nc(C)c(F)cc4c3P(C)(C)=O)n2)c(OC)cc1N1CCC(N2CC3(CN(c4cc(F)c([C@H]5CCC(=O)NC5=O)c(F)c4)C3)C2)CC1]

The title compound was prepared in a procedure similar to that in Example 9. ¹H NMR (500 MHz, DMSO) δ 11.29 (s, 1H), 10.85 (s, 1H), 8.63 (d, J=12.4 Hz, 1H), 8.20 (d, J=19.9 Hz, 2H), 7.94 (d, J=12.6 Hz, 2H), 7.32 (s, 1H), 6.70 (s, 1H), 6.14 (d, J=11.1 Hz, 2H), 4.03 (dd, J=12.7, 4.7 Hz, 1H), 3.91 (s, 4H), 3.75 (s, 3H), 3.28 (s, 3H), 2.85 (d, J=11.6 Hz, 2H), 2.78 (t, J=12.9 Hz, 1H), 2.64 (d, J=2.1 Hz, 3H), 2.58 (t, J=10.2 Hz, 4H), 2.09 (dd, J=39.2, 26.0 Hz, 4H), 1.95 (d, J=13.4 Hz, 7H), 1.69 (s, 2H), 1.29 (d, J=9.9 Hz, 2H), 0.70 (s, 3H). [M+H]⁺=960.6.

### Example 61: (R)-3-(4-(4-(6-(4-((5-chloro-4-((5-(dimethylphosphoryl)-3-fluoro-2-methylquinolin-6-yl)amino)pyrimidin-2-yl)amino)-2-ethyl-5-methoxyphenyl)-2,6-diazaspiro[3.3]heptan-2-yl)piperidin-1-yl)-2,6-difluorophenyl)piperidine-2,6-dione

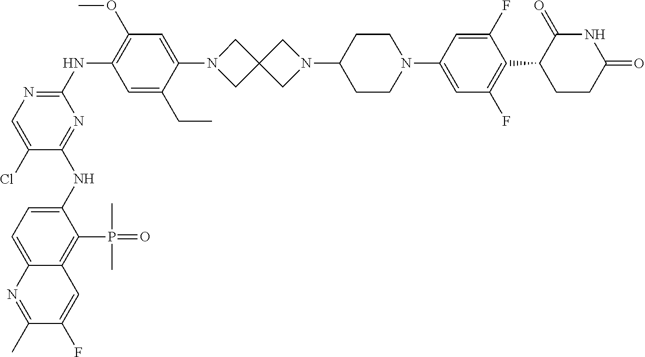 [SMILES: CCc1cc(Nc2ncc(Cl)c(Nc3ccc4nc(C)c(F)cc4c3P(C)(C)=O)n2)c(OC)cc1N1CC2(C1)CN(C1CCN(c3cc(F)c([C@H]4CCC(=O)NC4=O)c(F)c3)CC1)C2]

The titled compound (30 mg, 40%) was prepared in a manner similar to that in Example 1. ¹H NMR (500 MHz, CDCl3) δ 12.17 (s, 1H), 8.82 (d, J=6.5 Hz, 1H), 8.12 (s, 1H), 8.03 (d, J=9.5 Hz, 1H), 7.92 (s, 1H), 7.79 (s, 1H), 7.64 (d, J=11.5 Hz, 1H), 7.15 (s, 1H), 6.41 (d, J=12.0 Hz, 2H), 6.07 (s, 1H), 4.02-3.90 (m, 5H), 3.83 (s, 3H), 3.64-3.58 (m, 1H), 3.47-3.30 (m, 3H), 2.77-2.90 (m, 3H), 2.75-2.62 (m, 4H), 2.45-2.27 (m, 3H), 2.20-2.06 (m, 8H), 1.85-1.70 (m, 10.4 Hz, 1H), 1.61-1.51 (m, 2H), 1.45-1.35 (m, 2H), 1.03-0.80 (m, 4H). [M+H]⁺=916.70.

### Example 62: (R)-3-(4-(4-(7-(4-((5-bromo-4-((5-(dimethylphosphoryl)-3-fluoro-2-methylquinolin-6-yl)amino)pyrimidin-2-yl)amino)-2-ethyl-5-methoxyphenyl)-2,7-diazaspiro[3.5]nonan-2-yl)piperidin-1-yl)-2,6-difluorophenyl)piperidine-2,6-dione

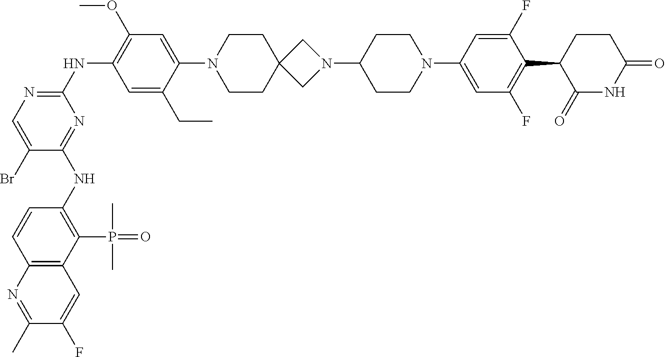 [SMILES: CCc1cc(Nc2ncc(Br)c(Nc3ccc4nc(C)c(F)cc4c3P(C)(C)=O)n2)c(OC)cc1N1CCC2(CC1)CN(C1CCN(c3cc(F)c([C@H]4CCC(=O)NC4=O)c(F)c3)CC1)C2]

The title compound was prepared in a procedure similar to that in Example 9. ¹H NMR (500 MHz, DMSO) δ 11.30 (s, 1H), 10.87 (s, 1H), 8.64 (s, 1H), 8.27-8.15 (m, 2H), 8.01-7.89 (m, 2H), 7.32 (s, 1H), 6.78-6.62 (m, 3H), 4.04 (dd, J=11.8, 4.8 Hz, 1H), 3.86-3.72 (m, 5H), 3.31-3.24 (m, 2H), 2.87-2.67 (m, 8H), 2.67-2.57 (m, 4H), 2.39-2.49 (m, 2H), 2.26-2.15 (m, 2H), 2.12-2.03 (m, 1H), 1.99-1.88 (m, 7H), 1.87-1.62 (m, 4H), 1.48-1.28 (m, 4H), 0.71 (s, 3H); [M+H]⁺=988.4.

### Example 63: 3-(4-(1-(1′-(4-((5-bromo-4-((5-(dimethylphosphoryl)-2-ethylquinolin-6-yl)amino)pyrimidin-2-yl)amino)-2-ethyl-5-methoxyphenyl)-[1,4′-bipiperidin]-4-yl)azetidin-3-yl)-3-methyl-2-oxo-2,3-dihydro-1H-benzo[d]imidazol-1-yl)piperidine-2,6-dione

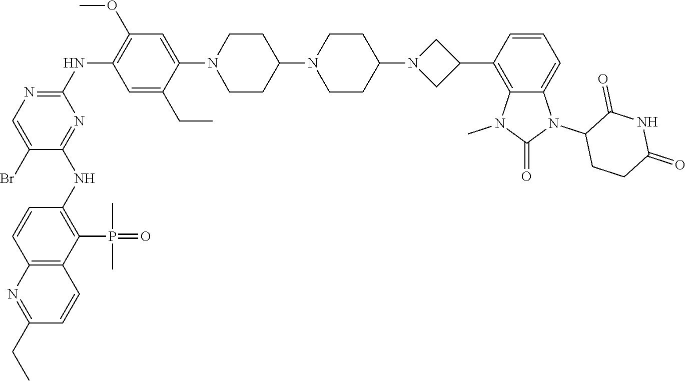 [SMILES: CCc1ccc2c(P(C)(C)=O)c(Nc3nc(Nc4cc(CC)c(N5CCC(N6CCC(N7CC(c8cccc9c8n(C)c(=O)n9C8CCC(=O)NC8=O)C7)CC6)CC5)cc4OC)ncc3Br)ccc2n1]

The title compound was prepared in a procedure similar to that in Example 25. 1H NMR (400 MHz, DMSO) δ 11.79 (s, 1H), 11.09 (s, 1H), 8.56 (d, J=8.8 Hz, 1H), 8.27 (s, 1H), 8.21 (s, 1H), 8.00 (s, 1H), 7.87 (d, J=9.3 Hz, 1H), 7.44 (d, J=8.9 Hz, 1H), 7.33 (s, 1H), 7.17 (d, J=7.8 Hz, 1H), 7.08-6.99 (m, 2H), 6.74 (s, 1H), 5.37 (dd, J=12.6, 5.1 Hz, 1H), 4.22-4.16 (m, 1H), 3.76 (s, 3H), 3.62 (t, J=6.8 Hz, 2H), 3.53 (s, 3H), 3.18 (s, 4H), 2.94 (t, J=7.6 Hz, 4H), 2.86 (d, J=6.2 Hz, 3H), 2.69-2.62 (m, 3H), 2.38 (d, J=11.7 Hz, 1H), 2.28-2.22 (m, 3H), 2.07 (s, 1H), 1.98 (d, J=13.3 Hz, 7H), 1.81 (d, J=11.1 Hz, 2H), 1.70 (d, J=10.6 Hz, 2H), 1.58 (d, J=9.2 Hz, 2H), 1.32 (t, J=7.6 Hz, 3H), 1.21 (d, J=10.0 Hz, 2H), 0.77 (s, 3H); [M+H]⁺=1032.7

### Example 64: (R)-3-(4-(4-(6-(4-((5-bromo-4-((5-(dimethylphosphoryl)-3-fluoro-2-methylquinolin-6-yl)amino)pyrimidin-2-yl)amino)-2-ethyl-5-methoxyphenyl)-2,6-diazaspiro[3.3]heptan-2-yl)piperidin-1-yl)-2,6-difluorophenyl)piperidine-2,6-dione

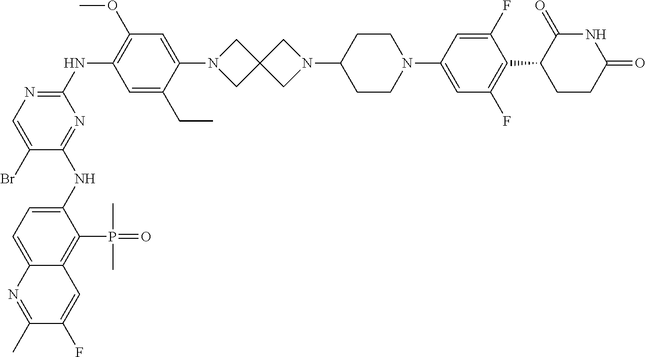 [SMILES: CCc1cc(Nc2ncc(Br)c(Nc3ccc4nc(C)c(F)cc4c3P(C)(C)=O)n2)c(OC)cc1N1CC2(C1)CN(C1CCN(c3cc(F)c([C@H]4CCC(=O)NC4=O)c(F)c3)CC1)C2]

The titled compound was prepared in a manner similar to that in Example 1. 1H NMR (500 MHz, DMSO) δ 11.59 (s, 1H), 10.87 (s, 1H), 8.46 (d, J=12.5 Hz, 1H), 8.31 (s, 1H), 8.17 (s, 1H), 7.97 (s, 1H), 7.88 (d, J=8.8 Hz, 1H), 7.04 (s, 1H), 6.62 (d, J=13.0 Hz, 2H), 6.11 (s, 1H), 4.04 (dd, J=12.5, 5.0 Hz, 1H), 3.88 (s, 4H), 3.73 (s, 3H), 3.60 (s, 2H), 3.35-3.31 (m, 5H), 2.91-2.73 (m, 3H), 2.63 (d, J=2.0 Hz, 3H), 2.25-2.05 (m, 4H), 2.02-1.92 (m, 7H), 1.75-1.60 (m, 2H), 1.30-1.15 (m, 2H), 0.82 (s, 3H). [M+H]⁺=960.7.

### Example 65: (R)-3-(4-(4-(6-(4-((5-bromo-4-((5-(dimethylphosphoryl)-3-fluoro-2-methylquinolin-6-yl)amino)pyrimidin-2-yl)amino)-5-methoxy-2-methylphenyl)-2,6-diazaspiro[3.3]heptan-2-yl)piperidin-1-yl)-2,6-difluorophenyl)piperidine-2,6-dione

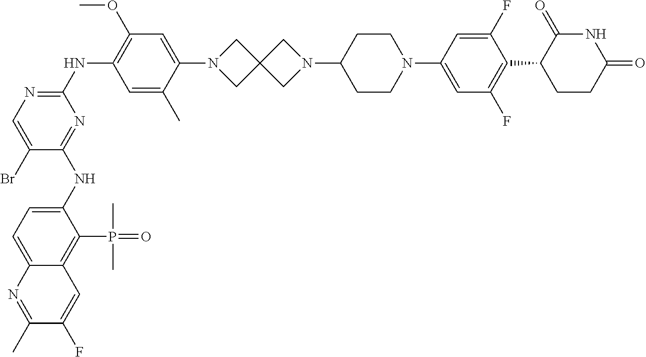 [SMILES: COc1cc(N2CC3(C2)CN(C2CCN(c4cc(F)c([C@H]5CCC(=O)NC5=O)c(F)c4)CC2)C3)c(C)cc1Nc1ncc(Br)c(Nc2ccc3nc(C)c(F)cc3c2P(C)(C)=O)n1]

The titled compound was prepared in a manner similar to that in Example 1. 1H NMR (500 MHz, DMSO) δ 11.76 (s, 1H), 10.87 (s, 1H), 8.43-8.33 (m, 2H), 8.17 (s, 1H), 7.97 (s, 1H), 7.88 (d, J=8.5 Hz, 1H), 7.04 (s, 1H), 6.63 (d, J=13.0 Hz, 2H), 6.12 (s, 1H), 4.04 (dd, J=12.5, 5.0 Hz, 1H), 3.90 (s, 4H), 3.73 (s, 3H), 3.66-3.55 (s, 2H), 3.32 (s, 6H), 2.90-2.75 (m, 3H), 2.63 (d, J=2.0 Hz, 3H), 2.21-2.04 (m, 2H), 2.01-1.92 (m, 8H), 1.84 (s, 3H), 1.72-1.65 (m, 2H). [M+H]⁺=946.7.

### Example 66: (R)-3-(4-(4-(9-(4-((5-bromo-4-((5-(dimethylphosphoryl)-2-methylquinazolin-6-yl)amino)pyrimidin-2-yl)amino)-5-methoxy-2-methylphenyl)-3,9-diazaspiro[5.5]undecan-3-yl)piperidin-1-yl)-2,6-difluorophenyl)piperidine-2,6-dione

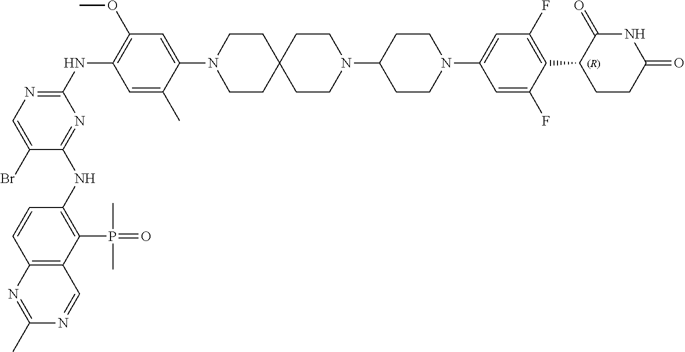

The title compound was prepared in a manner similar to that in Example 1. ¹H NMR (500 MHz, DMSO) δ 11.75 (s, 1H), 10.86 (s, 1H), 9.77 (s, 1H), 8.54 (s, 1H), 8.22 (s, 1H), 8.05 (s, 1H), 7.85 (d, J=9.6 Hz, 1H), 7.24 (s, 1H), 6.74 (s, 1H), 6.63 (d, J=12.9 Hz, 2H), 4.04 (dd, J=12.9, 4.8 Hz, 1H), 3.80 (d, J=12.0 Hz, 2H), 3.75 (s, 3H), 2.85-2.67 (m, 10H), 2.36 (s, 4H), 2.15-1.92 (m, 9H), 1.89 (s, 3H), 1.81 (d, J=11.2 Hz, 2H), 1.59-1.42 (m, 10H), 1.23 (s, 1H). [M+H]⁺=987.6.

### Example 68: 3-(4-(2-(9-(4-((5-bromo-4-((5-(dimethylphosphoryl)-2-ethylquinolin-6-yl)amino)pyrimidin-2-yl)amino)-5-methoxy-2-methylphenyl)-3,9-diazaspiro[5.5]undecan-3-yl)ethyl)-3-methyl-2-oxo-2,3-dihydro-1H-benzol[d]imidazol-1-yl)piperidine-2,6-dione

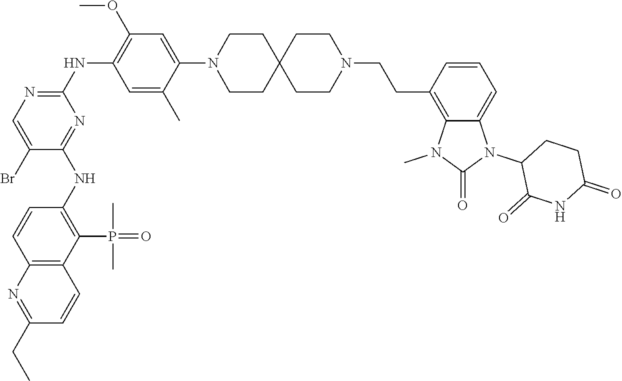 [SMILES: CCc1ccc2c(P(C)(C)=O)c(Nc3nc(Nc4cc(C)c(N5CCC6(CCN(CCc7cccc8c7n(C)c(=O)n8C7CCC(=O)NC7=O)CC6)CC5)cc4OC)ncc3Br)ccc2n1]

The title compound was prepared in a manner similar to that in Example 28. ¹H NMR (500 MHz, DMSO) δ 11.93 (s, 1H), 11.09 (s, 1H), 8.50 (s, 1H), 8.35 (s, 1H), 8.21 (d, J=18.5 Hz, 1H), 8.03 (s, 1H), 7.87 (s, 1H), 7.43 (d, J=9.0 Hz, 1H), 7.25 (s, 1H), 6.95 (d, J=31.2 Hz, 3H), 6.77 (s, 1H), 5.36 (s, 1H), 3.77 (s, 3H), 3.59 (s, 3H), 3.08 (s, 2H), 2.95-2.69 (m, 8H), 2.61 (d, J=24.6 Hz, 7H), 2.04-1.87 (m, 10H), 1.57 (s, 8H), 1.39-1.26 (m, 3H). [M+H]⁺=977.7.

### Example 72: 3-(5-(4-((9-(4-((5-bromo-4-((5-(dimethylphosphoryl)-2-methylquinolin-6-yl)amino)pyrimidin-2-yl)amino)-5-methoxy-2-methylphenyl)-3,9-diazaspiro[5.5]undecan-3-yl)methyl)piperidin-1-yl)-1-oxoisoindolin-2-yl)piperidine-2,6-dione

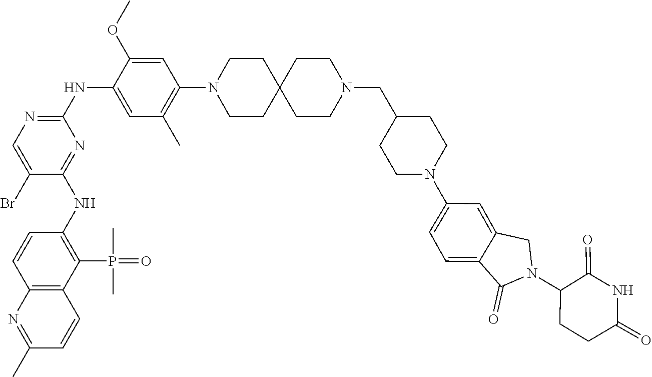 [SMILES: COc1cc(N2CCC3(CCN(CC4CCN(c5ccc6c(c5)CN(C5CCC(=O)NC5=O)C6=O)CC4)CC3)CC2)c(C)cc1Nc1ncc(Br)c(Nc2ccc3nc(C)ccc3c2P(C)(C)=O)n1]

The title compound (30 mg, 40.5%) was prepared in a manner similar to that in Example 33. ¹H NMR (500 MHz, DMSO) δ 11.93 (s, 1H), 10.94 (s, 1H), 8.50 (d, J=8.9 Hz, 1H), 8.39 (d, J=5.7 Hz, 1H), 8.20 (s, 1H), 7.99 (s, 1H), 7.88 (d, J=9.4 Hz, 1H), 7.50 (d, J=8.4 Hz, 1H), 7.42 (d, J=8.9 Hz, 1H), 7.32 (s, 1H), 7.05 (d, J=8.5 Hz, 2H), 6.75 (s, 1H), 5.04 (dd, J=13.2, 5.1 Hz, 1H), 4.32 (d, J=16.9 Hz, 1H), 4.20 (d, J=16.9 Hz, 1H), 3.88 (d, J=12.2 Hz, 2H), 3.76 (s, 3H), 2.96-2.81 (m, 3H), 2.79 (d, J=16.0 Hz, 5H), 2.64 (s, 3H), 2.59 (d, J=16.5 Hz, 2H), 2.54 (s, 2H), 2.42-2.32 (m, 2H), 2.29 (s, 2H), 1.99 (d, J=13.3 Hz, 6H), 1.94 (d, J=16.8 Hz, 4H), 1.79 (d, J=10.7 Hz, 3H), 1.58 (s, 7H), 1.20 (d, J=10.6 Hz, 2H). [M+H]⁺=1017.4.

### Example 73: (R)-3-(4-(4-(9-(4-((5-bromo-4-((5-(dimethylphosphoryl)-2-methylquinolin-6-yl)amino)pyrimidin-2-yl)amino)-5-methoxy-2-methylphenyl)-3,9-diazaspiro[5.5]undecan-3-yl)piperidin-1-yl)-2,6-difluorophenyl)piperidine-2,6-dione

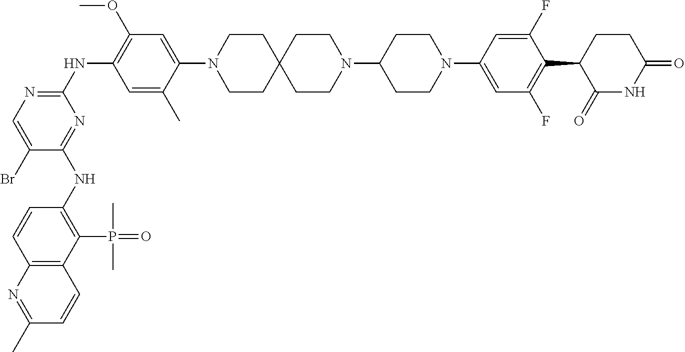 [SMILES: COc1cc(N2CCC3(CC2)CCN(C2CCN(c4cc(F)c([C@H]5CCC(=O)NC5=O)c(F)c4)CC2)CC3)c(C)cc1Nc1ncc(Br)c(Nc2ccc3nc(C)ccc3c2P(C)(C)=O)n1]

The title compound was prepared in a manner similar to that in Example 1. ¹H NMR (500 MHz, DMSO) δ 11.94 (s, 1H), 10.87 (s, 1H), 8.50 (d, J=8.5 Hz, 1H), 8.38 (s, 1H), 8.20 (s, 1H), 8.00 (s, 1H), 7.87 (d, J=8.3 Hz, 1H), 7.41 (s, 1H), 7.30 (s, 1H), 6.75 (s, 1H), 6.63 (d, J=12.8 Hz, 2H), 4.04 (s, 1H), 3.77 (d, J=14.7 Hz, 5H), 3.32 (s, 6H), 2.76 (s, 7H), 2.63 (s, 3H), 2.06 (d, J=20.0 Hz, 1H), 1.99 (d, J=13.3 Hz, 6H), 1.92 (s, 3H), 1.81 (s, 2H), 1.54 (s, 11H). [M+H]⁺=984.3.

### Example 79: (R)-3-(4-(4-((9-(4-((5-bromo-4-((5-(dimethylphosphoryl)-3-fluoro-2-methylquinolin-6-yl)amino)pyrimidin-2-yl)amino)-2-ethyl-5-methoxyphenyl)-3,9-diazaspiro[5.5]undecan-3-yl)methyl)piperidin-1-yl)-2,6-difluorophenyl)piperidine-2,6-dione

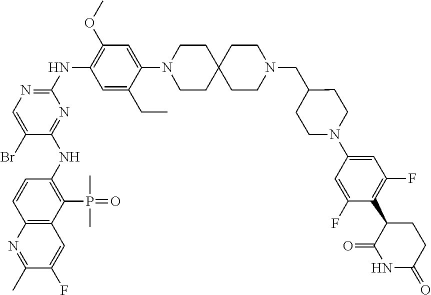 [SMILES: CCc1cc(Nc2ncc(Br)c(Nc3ccc4nc(C)c(F)cc4c3P(C)(C)=O)n2)c(OC)cc1N1CCC2(CCN(CC3CCN(c4cc(F)c([C@H]5CCC(=O)NC5=O)c(F)c4)CC3)CC2)CC1]

The title compound was prepared in a procedure similar to that in Example 1. ¹H NMR (500 MHz, DMSO) δ_{H} 11.34 (s, 1H), 10.86 (s, 1H), 8.60 (d, J=12.2 Hz, 1H), 8.22 (s, 2H), 7.94 (d, J=14.4 Hz, 2H), 7.30 (s, 1H), 6.78 (s, 1H), 6.60 (d, J=12.9 Hz, 2H), 4.03 (d, J=12.8 Hz, 1H), 3.78-3.70 (m, 5H), 2.78-2.68 (m, 7H), 2.63 (s, 3H), 2.38-2.31 (m, 4H), 2.22-2.05 (m, 6H), 1.98-1.91 (m, 7H), 1.74 (d, J=11.7 Hz, 3H), 1.58-1.48 (m, 8H), 1.12 (d, J=11.0 Hz, 2H), 0.72 (s, 3H). [M+H]⁺=1030.6.

### Example 83: (S)-3-(4-(3-(9-(4-((5-bromo-4-((5-(dimethylphosphoryl)-3-fluoro-2-methylquinolin-6-yl)amino)pyrimidin-2-yl)amino)-2-ethyl-5-methoxyphenyl)-3,9-diazaspiro[5.5]undecan-3-yl)propyl)-7-fluoro-3-methyl-2-oxo-2,3-dihydro-1H-benzo[d]imidazol-1-yl)piperidine-2,6-dione

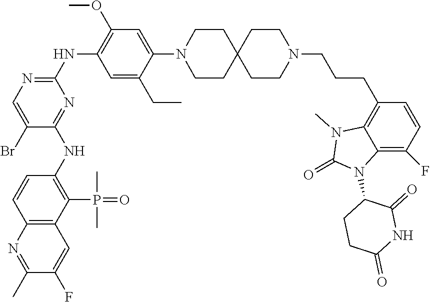 [SMILES: CCc1cc(Nc2ncc(Br)c(Nc3ccc4nc(C)c(F)cc4c3P(C)(C)=O)n2)c(OC)cc1N1CCC2(CCN(CCCc3ccc(F)c4c3n(C)c(=O)n4[C@H]3CCC(=O)NC3=O)CC2)CC1]

The title compound was prepared in a procedure similar to that in Example 28. ¹H NMR (500 MHz, DMSO) δ 11.34 (s, 1H), 11.10 (s, 1H), 8.60 (d, J=11.8 Hz, 1H), 8.22 (s, 2H), 7.99-7.89 (m, 2H), 7.30 (s, 1H), 6.88 (t, J=7.7 Hz, 2H), 6.78 (s, 1H), 5.52 (d, J=8.3 Hz, 1H), 3.75 (s, 3H), 3.63 (s, 2H), 3.00 (s, 1H), 2.94-2.85 (m, 2H), 2.74-2.69 (m, 4H), 2.64 (s, 3H), 2.60 (s, 1H), 2.44-2.38 (m, 6H), 2.19 (s, 3H), 2.09 (s, 1H), 1.96 (s, 3H), 1.94 (s, 3H), 1.75 (s, 2H), 1.56-1.48 (m, 8H), 1.23 (s, 1H), 0.72 (s, 3H). [M+H]⁺=1027.7.

### Example 85: 3-(7-(2-(9-(4-((5-bromo-4-((5-(dimethylphosphoryl)-3-fluoro-2-methylquinolin-6-yl)amino)pyrimidin-2-yl)amino)-2-ethyl-5-methoxyphenyl)-3,9-diazaspiro[5.5]undecan-3-yl)ethyl)-2-oxobenzo[d]oxazol-3(2H)-yl)piperidine-2,6-dione

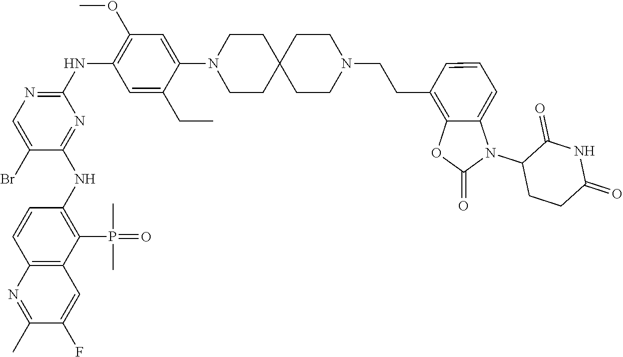 [SMILES: CCc1cc(Nc2ncc(Br)c(Nc3ccc4nc(C)c(F)cc4c3P(C)(C)=O)n2)c(OC)cc1N1CCC2(CCN(CCc3cccc4c3oc(=O)n4C3CCC(=O)NC3=O)CC2)CC1]

The title compound was prepared in a procedure similar to that in Example 28. ¹H NMR (500 MHz, DMSO) δ 11.35 (s, 1H), 11.21 (s, 1H), 8.59 (d, J=11.9 Hz, 1H), 8.22 (s, 2H), 8.01-7.90 (m, 2H), 7.30 (s, 1H), 7.17-7.02 (m, 3H), 6.80 (s, 1H), 5.36 (dd, J=12.9, 5.2 Hz, 1H), 3.76 (s, 3H), 2.87 (d, J=8.1 Hz, 4H), 2.76-2.59 (m, 13H), 2.48-2.44 (m, 1H), 2.27-2.13 (m, 3H), 1.97 (s, 3H), 1.94 (s, 3H), 1.58-1.50 (m, 8H), 0.72 (s, 3H). [M+H]⁺=982.7.

### Example 86: 3-(4′-(2-(9-(4-((5-bromo-4-((5-(dimethylphosphoryl)-3-fluoro-2-methylquinolin-6-yl)amino)pyrimidin-2-yl)amino)-2-ethyl-5-methoxyphenyl)-3,9-diazaspiro[5.5]undecan-3-yl)ethyl)-2′-oxospiro[cyclopropane-1,3′-indolin]-1′-yl)piperidine-2,6-dione

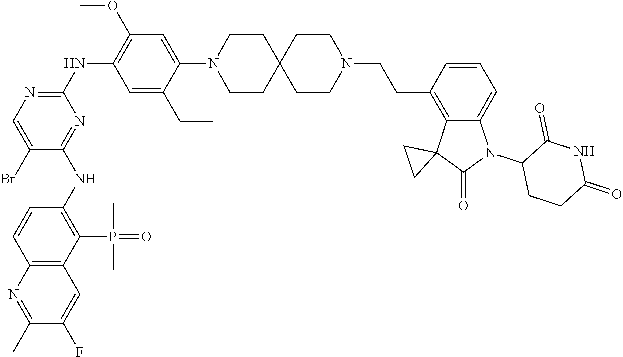 [SMILES: CCc1cc(Nc2ncc(Br)c(Nc3ccc4nc(C)c(F)cc4c3P(C)(C)=O)n2)c(OC)cc1N1CCC2(CCN(CCc3cccc4c3C3(CC3)C(=O)N4C3CCC(=O)NC3=O)CC2)CC1]

The title compound was prepared in a manner similar to that in Example 52. ¹H NMR (500 MHz, DMSO) δ 11.35 (s, 1H), 11.08 (s, 1H), 8.60 (d, J=12.0 Hz, 1H), 8.22 (s, 2H), 7.99-7.91 (m, 2H), 7.30 (s, 1H), 7.16 (t, J=7.9 Hz, 1H), 6.87 (d, J=7.8 Hz, 2H), 6.80 (s, 1H), 5.31 (s, 1H), 3.76 (s, 3H), 2.86 (d, J=13.1 Hz, 1H), 2.72 (s, 4H), 2.66-2.55 (m, 6H), 2.49-2.45 (m, 5H), 2.20 (s, 2H), 2.00-1.91 (m, 10H), 1.60-1.44 (m, 11H), 0.72 (s, 3H). [M+H]⁺=1006.8.

### Example 87: 3-(5-(4-(9-(4-((5-bromo-4-((5-(dimethylphosphoryl)-3-fluoro-2-methylquinolin-6-yl)amino)pyrimidin-2-yl)amino)-2-ethyl-5-methoxyphenyl)-3,9-diazaspiro[5.5]undecan-3-yl)piperidin-1-yl)-1-oxoisoindolin-2-yl)piperidine-2,6-dione

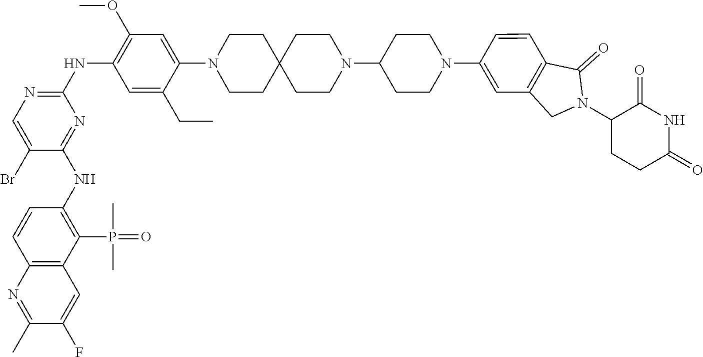 [SMILES: CCc1cc(Nc2ncc(Br)c(Nc3ccc4nc(C)c(F)cc4c3P(C)(C)=O)n2)c(OC)cc1N1CCC2(CC1)CCN(C1CCN(c3ccc4c(c3)CN(C3CCC(=O)NC3=O)C4=O)CC1)CC2]

The title compound was prepared in a manner similar to that in Example 33. ¹H NMR (500 MHz, DMSO) δ 11.34 (s, 1H), 10.94 (s, 1H), 8.59 (s, 1H), 8.21 (s, 2H), 8.00-7.91 (m, 2H), 7.50 (d, J=8.4 Hz, 1H), 7.30 (s, 1H), 7.05 (d, J=9.4 Hz, 2H), 6.78 (s, 1H), 5.04 (dd, J=13.4, 5.0 Hz, 1H), 4.32 (d, J=16.9 Hz, 1H), 4.20 (d, J=16.7 Hz, 1H), 3.91 (d, J=12.1 Hz, 2H), 3.75 (s, 3H), 2.98-2.77 (m, 4H), 2.71 (s, 4H), 2.61 (dd, J=24.8, 9.4 Hz, 6H), 2.49-2.43 (m, 3H), 2.41-2.33 (m, 1H), 2.19 (s, 2H), 1.95 (d, J=13.4 Hz, 7H), 1.84 (d, J=10.7 Hz, 2H), 1.52 (s, 9H), 0.71 (s, 3H). [M+H]⁺=1035.8.

### Example 90: 3-(5-((2-(9-(4-((5-bromo-4-((5-(dimethylphosphoryl)-3-fluoro-2-methylquinolin-6-yl)amino)pyrimidin-2-yl)amino)-2-ethyl-5-methoxyphenyl)-3,9-diazaspiro[5.5]undecan-3-yl)ethyl)(methyl)amino)-1-oxoisoindolin-2-yl)piperidine-2,6-dione

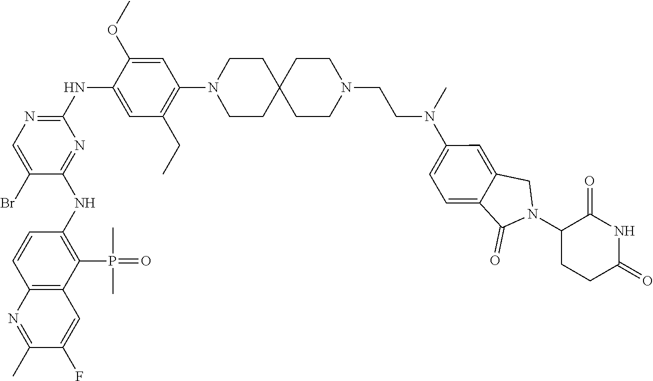 [SMILES: CCc1cc(Nc2ncc(Br)c(Nc3ccc4nc(C)c(F)cc4c3P(C)(C)=O)n2)c(OC)cc1N1CCC2(CCN(CCN(C)c3ccc4c(c3)CN(C3CCC(=O)NC3=O)C4=O)CC2)CC1]

To a 40-mL microwave vial equipped with a magnetic stir bar was added (6-((5-bromo-2-((5-ethyl-2-methoxy-4-(3,9-diazaspiro[5.5]undecan-3-yl)phenyl)amino)pyrimidin-4-yl)amino)-3-fluoro-2-methylquinolin-5-yl)dimethylphosphine oxide (71.0 mg, 0.10 mmol), 2-((2-(2,6-dioxopiperidin-3-yl)-1-oxoisoindolin-5-yl)(methyl)amino)acetaldehyde (the compound was obtained through the similar way of Example 33 step 6) (63.2 mg, 0.20 mmol) and DCM (8 mL). After stirring for 20 min at room temperature, NaBH(OAc)₃ (63.6 mg, 0.3 mmol) was added. After stirring for another 1 H at the same temperature, the reaction mixture was diluted with sat. aq. NaHCO₃ (20 mL) and the layers were separated. The aqueous layer was extracted with DCM (3×15 mL). The combined organic layers were washed with brine (20 mL), dried over anhydrous Na₂SO₄, filtered and concentrated under reduced pressure. Purification by silica gel column chromatography followed by prep-HPLC chromatography (0.1% FA in water:acetonitrile=90:10˜50:50 gradient elution) to afford the title compound (52 mg, 52%). ¹H NMR (500 MHz, DMSO) δ 11.27 (s, 1H), 10.92 (s, 1H), 8.54 (d, J=9.5 Hz, 1H), 8.15-8.11 (m, 2H), 7.88 (s, 1H), 7.87 (d, J=9.5 Hz, 1H), 7.30 (t, J=8.0 Hz, 1H), 7.23 (s, 1H), 7.08 (d, J=7.5 Hz, 1H), 6.94 (d, J=8.5 Hz, 1H), 6.71 (s, 1H), 5.05 (dd, J=13.0, 5.0 Hz, 1H), 4.45 (d, J=16.5 Hz, 1H), 4.32 (d, J=16.5 Hz, 1H), 3.68 (s, 3H), 2.88 (s, 3H), 2.88-2.81 (m, 1H), 2.62 (s, 3H), 2.56 (d, J=2.0 Hz, 3H), 2.53-2.50 (m, 1H), 2.41-2.35 (m, 5H), 2.34-1.28 (m, 4H), 2.15-2.07 (m, 2H), 1.96-1.92 (m, 2H), 1.88 (d, J=13.5 Hz, 6H), 1.43-1.38 (m, 8H), 0.64 (t, J=8.0 Hz, 3H), [M+H]⁺=1009.8.

### Example 92: 5-(4-(9-(4-((5-bromo-4-((5-(dimethylphosphoryl)-3-fluoro-2-methylquinolin-6-yl)amino)pyrimidin-2-yl)amino)-2-ethyl-5-methoxyphenyl)-3,9-diazaspiro[5.5]undecan-3-yl)piperidin-1-yl)-2-(2,6-dioxopiperidin-3-yl)isoindoline-1,3-dione

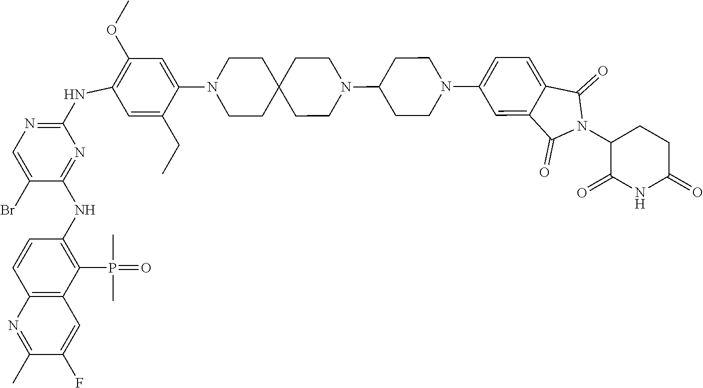 [SMILES: CCc1cc(Nc2ncc(Br)c(Nc3ccc4nc(C)c(F)cc4c3P(C)(C)=O)n2)c(OC)cc1N1CCC2(CC1)CCN(C1CCN(c3ccc4c(c3)C(=O)N(C3CCC(=O)NC3=O)C4=O)CC1)CC2]

The title compound was prepared in a manner similar to that in Example 33. ¹H NMR (500 MHz, DMSO) δ 11.07 (s, 1H), 8.61 (s, 1H), 8.22 (s, 2H), 7.98-7.90 (m, 2H), 7.66 (d, J=8.5 Hz, 1H), 7.28-7.24 (m, 3H), 6.78 (s, 1H), 5.10-5.05 (m, 1H), 4.09 (s, 2H), 3.75 (s, 3H), 2.97-2.88 (m, 4H), 2.71-2.63 (m, 16H), 2.19 (s, 2H), 1.95-1.85 (m, 8H), 1.85 (s, 2H), 1.54-1.52 (m, 8H), 0.72 (s, 2H). [M+H]⁺=1049.5.

### Other Examples

| Example | Structure | Method Similar To |
|---|---|---|
| Example 94 | 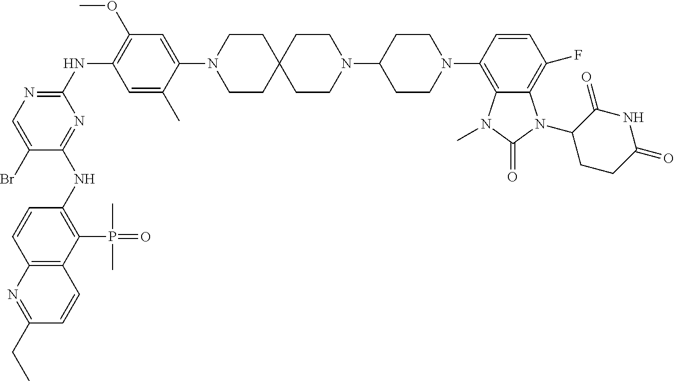 [SMILES: CCc1ccc2c(P(C)(C)=O)c(Nc3nc(Nc4cc(C)c(N5CCC6(CC5)CCN(C5CCN(c7ccc(F)c8c7n(C)c(=O)n8C7CCC(=O)NC7=O)CC5)CC6)cc4OC)ncc3Br)ccc2n1] | Example 21 |
| Example 95 | 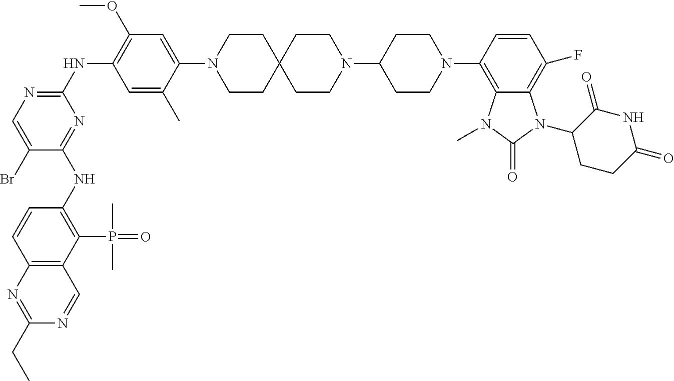 [SMILES: CCc1ncc2c(P(C)(C)=O)c(Nc3nc(Nc4cc(C)c(N5CCC6(CC5)CCN(C5CCN(c7ccc(F)c8c7n(C)c(=O)n8C7CCC(=O)NC7=O)CC5)CC6)cc4OC)ncc3Br)ccc2n1] | Example 21 |
| Example 97 | 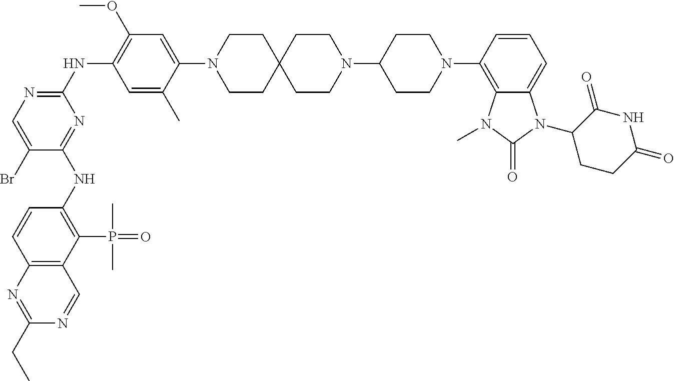 [SMILES: CCc1ncc2c(P(C)(C)=O)c(Nc3nc(Nc4cc(C)c(N5CCC6(CC5)CCN(C5CCN(c7cccc8c7n(C)c(=O)n8C7CCC(=O)NC7=O)CC5)CC6)cc4OC)ncc3Br)ccc2n1] | Example 21 |
| Example 98 | 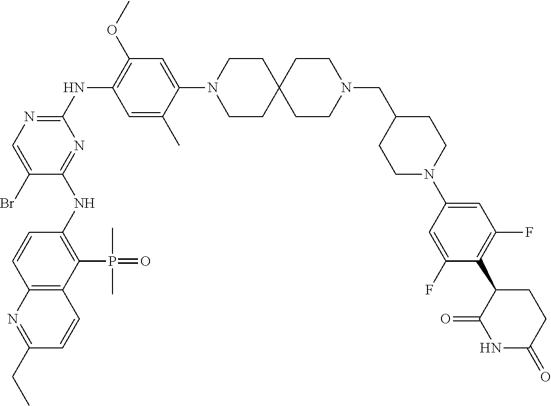 [SMILES: CCc1ccc2c(P(C)(C)=O)c(Nc3nc(Nc4cc(C)c(N5CCC6(CCN(CC7CCN(c8cc(F)c([C@H]9CCC(=O)NC9=O)c(F)c8)CC7)CC6)CC5)cc4OC)ncc3Br)ccc2n1] | Example 1 |
| Example 99 | 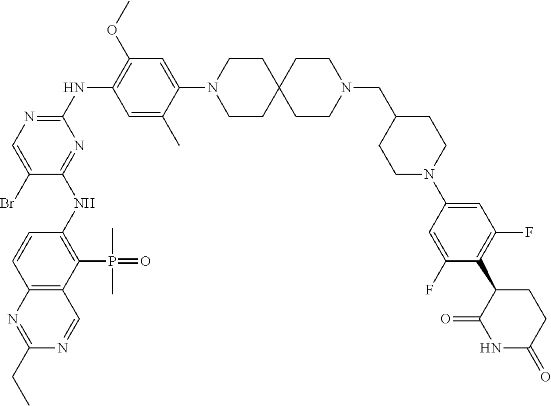 [SMILES: CCc1ncc2c(P(C)(C)=O)c(Nc3nc(Nc4cc(C)c(N5CCC6(CCN(CC7CCN(c8cc(F)c([C@H]9CCC(=O)NC9=O)c(F)c8)CC7)CC6)CC5)cc4OC)ncc3Br)ccc2n1] | Example 39 |
| Example 101 | 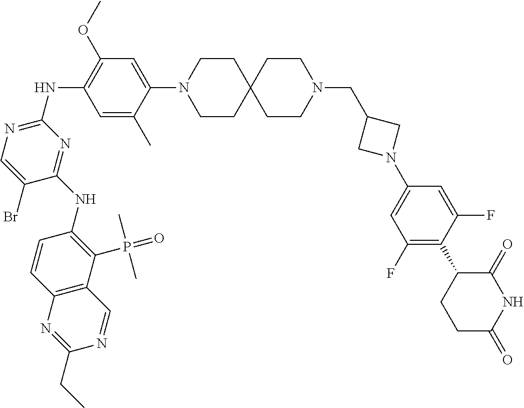 [SMILES: CCc1ncc2c(P(C)(C)=O)c(Nc3nc(Nc4cc(C)c(N5CCC6(CCN(CC7CN(c8cc(F)c([C@H]9CCC(=O)NC9=O)c(F)c8)C7)CC6)CC5)cc4OC)ncc3Br)ccc2n1] | Example 39 |
| Example 102 | 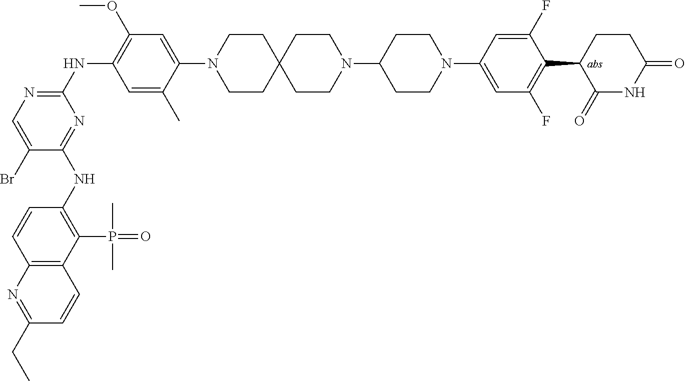 [SMILES: CCc1ccc2c(P(C)(C)=O)c(Nc3nc(Nc4cc(C)c(N5CCC6(CC5)CCN(C5CCN(c7cc(F)c([C@H]8CCC(=O)NC8=O)c(F)c7)CC5)CC6)cc4OC)ncc3Br)ccc2n1] | Example 1 |
| Example 103 | 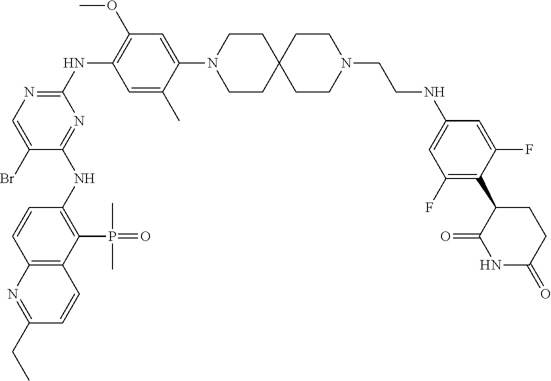 [SMILES: CCc1ccc2c(P(C)(C)=O)c(Nc3nc(Nc4cc(C)c(N5CCC6(CCN(CCNc7cc(F)c([C@H]8CCC(=O)NC8=O)c(F)c7)CC6)CC5)cc4OC)ncc3Br)ccc2n1] | Example 43 |
| Example 104 | 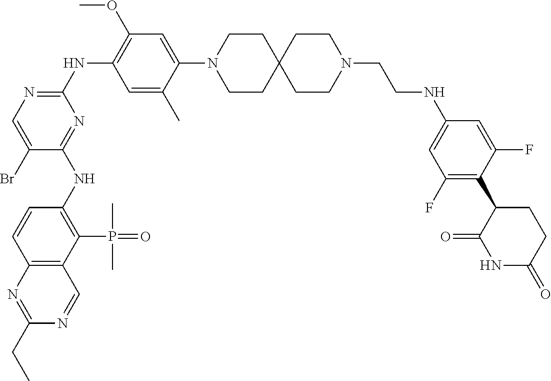 [SMILES: CCc1ncc2c(P(C)(C)=O)c(Nc3nc(Nc4cc(C)c(N5CCC6(CCN(CCNc7cc(F)c([C@H]8CCC(=O)NC8=O)c(F)c7)CC6)CC5)cc4OC)ncc3Br)ccc2n1] | Example 43 |
| Example 105 | 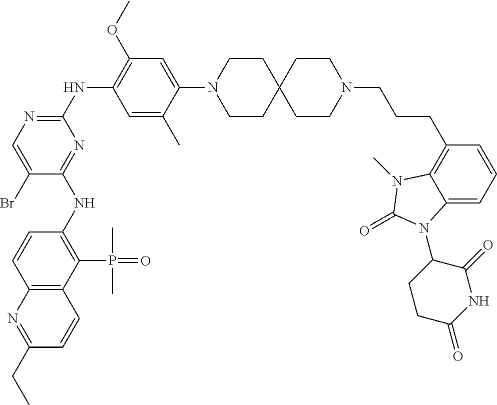 [SMILES: CCc1ccc2c(P(C)(C)=O)c(Nc3nc(Nc4cc(C)c(N5CCC6(CCN(CCCc7cccc8c7n(C)c(=O)n8C7CCC(=O)NC7=O)CC6)CC5)cc4OC)ncc3Br)ccc2n1] | Example 28 |
| Example 106 | 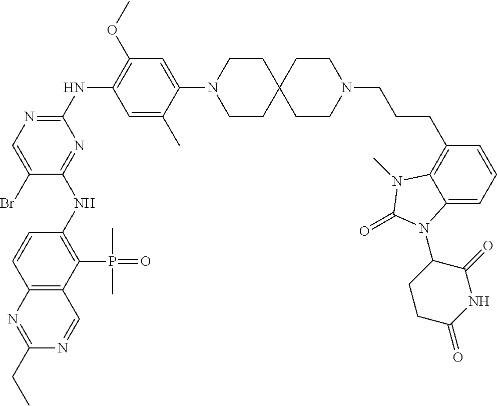 [SMILES: CCc1ncc2c(P(C)(C)=O)c(Nc3nc(Nc4cc(C)c(N5CCC6(CCN(CCCc7cccc8c7n(C)c(=O)n8C7CCC(=O)NC7=O)CC6)CC5)cc4OC)ncc3Br)ccc2n1] | Example 28 |
| Example 107 | 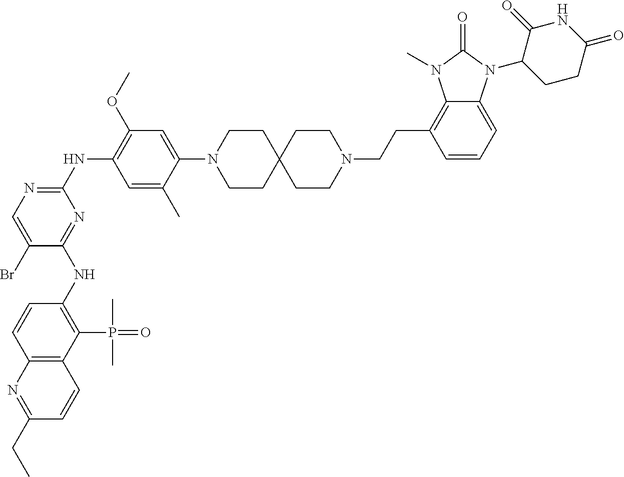 [SMILES: CCc1ccc2c(P(C)(C)=O)c(Nc3nc(Nc4cc(C)c(N5CCC6(CCN(CCc7cccc8c7n(C)c(=O)n8C7CCC(=O)NC7=O)CC6)CC5)cc4OC)ncc3Br)ccc2n1] | Example 28 |
| Example 108 | 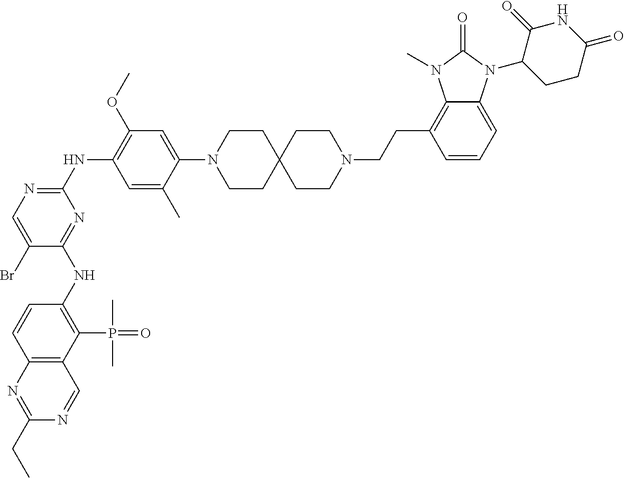 [SMILES: CCc1ncc2c(P(C)(C)=O)c(Nc3nc(Nc4cc(C)c(N5CCC6(CCN(CCc7cccc8c7n(C)c(=O)n8C7CCC(=O)NC7=O)CC6)CC5)cc4OC)ncc3Br)ccc2n1] | Example 28 |
| Example 109 | 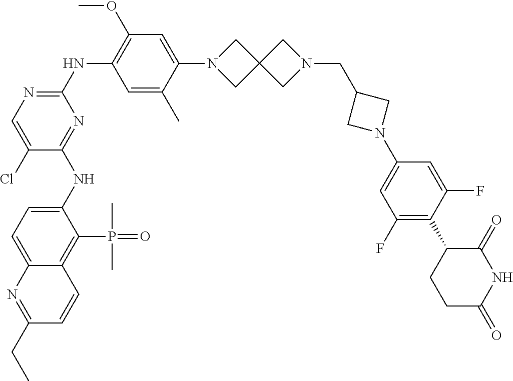 [SMILES: CCc1ccc2c(P(C)(C)=O)c(Nc3nc(Nc4cc(C)c(N5CC6(CN(CC7CN(c8cc(F)c([C@H]9CCC(=O)NC9=O)c(F)c8)C7)C6)C5)cc4OC)ncc3Cl)ccc2n1] | Example 39 |
| Example 110 | 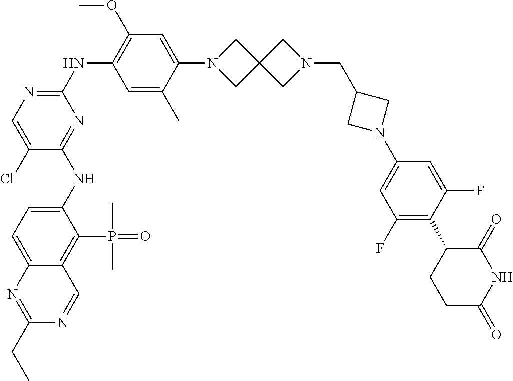 [SMILES: CCc1ncc2c(P(C)(C)=O)c(Nc3nc(Nc4cc(C)c(N5CC6(CN(CC7CN(c8cc(F)c([C@H]9CCC(=O)NC9=O)c(F)c8)C7)C6)C5)cc4OC)ncc3Cl)ccc2n1] | Example 39 |
| Example 111 | 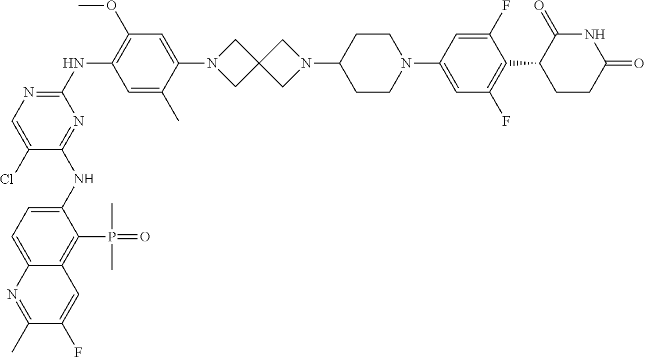 [SMILES: COc1cc(N2CC3(C2)CN(C2CCN(c4cc(F)c([C@H]5CCC(=O)NC5=O)c(F)c4)CC2)C3)c(C)cc1Nc1ncc(Cl)c(Nc2ccc3nc(C)c(F)cc3c2P(C)(C)=O)n1] | Example 1 |
| Example 112 | 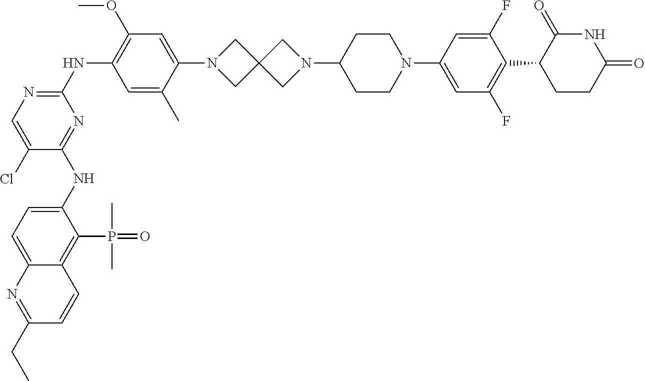 [SMILES: CCc1ccc2c(P(C)(C)=O)c(Nc3nc(Nc4cc(C)c(N5CC6(C5)CN(C5CCN(c7cc(F)c([C@H]8CCC(=O)NC8=O)c(F)c7)CC5)C6)cc4OC)ncc3Cl)ccc2n1] | Example 1 |
| Example 113 | 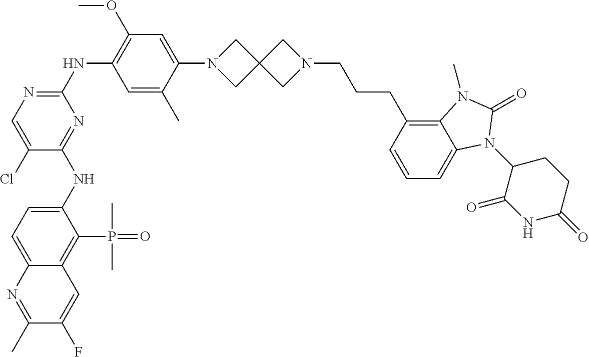 [SMILES: COc1cc(N2CC3(CN(CCCc4cccc5c4n(C)c(=O)n5C4CCC(=O)NC4=O)C3)C2)c(C)cc1Nc1ncc(Cl)c(Nc2ccc3nc(C)c(F)cc3c2P(C)(C)=O)n1] | Example 28 |
| Example 114 | 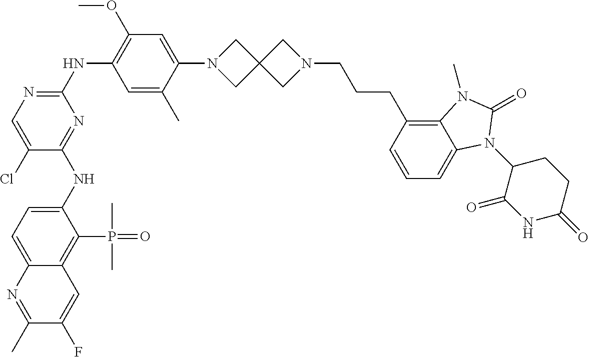 [SMILES: COc1cc(N2CC3(CN(CCCc4cccc5c4n(C)c(=O)n5C4CCC(=O)NC4=O)C3)C2)c(C)cc1Nc1ncc(Cl)c(Nc2ccc3nc(C)c(F)cc3c2P(C)(C)=O)n1] | Example 28 |
| Example 115 | 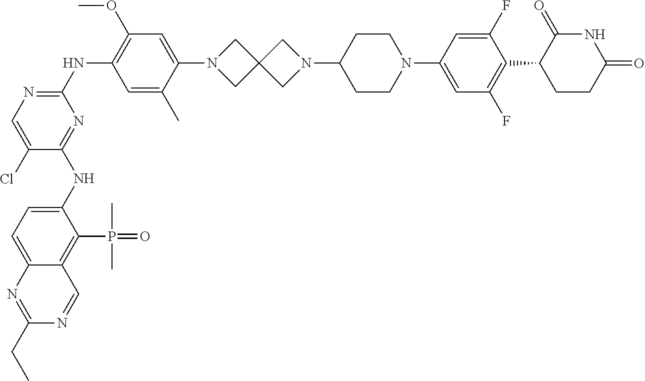 [SMILES: CCc1ncc2c(P(C)(C)=O)c(Nc3nc(Nc4cc(C)c(N5CC6(C5)CN(C5CCN(c7cc(F)c([C@H]8CCC(=O)NC8=O)c(F)c7)CC5)C6)cc4OC)ncc3Cl)ccc2n1] | Example 1 |
| Example 116 | 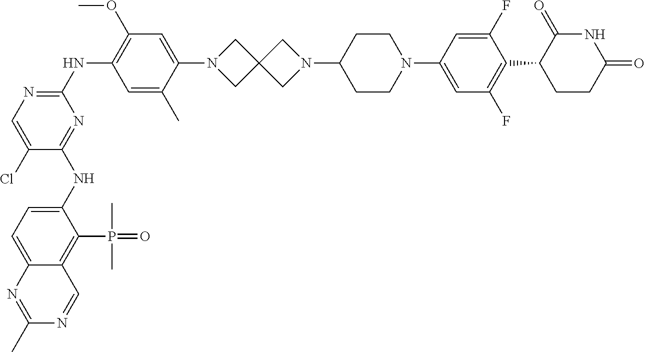 [SMILES: COc1cc(N2CC3(C2)CN(C2CCN(c4cc(F)c([C@H]5CCC(=O)NC5=O)c(F)c4)CC2)C3)c(C)cc1Nc1ncc(Cl)c(Nc2ccc3nc(C)ncc3c2P(C)(C)=O)n1] | Example 1 |
| Example 117 | 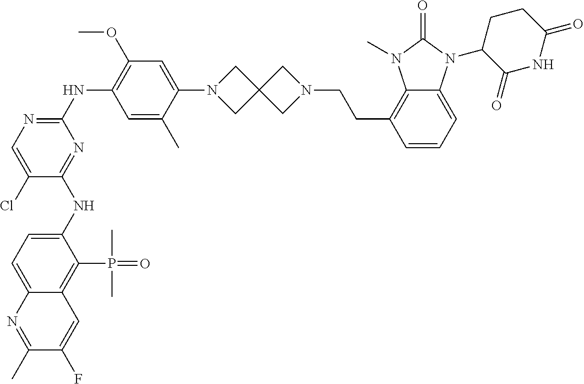 [SMILES: COc1cc(N2CC3(CN(CCc4cccc5c4n(C)c(=O)n5C4CCC(=O)NC4=O)C3)C2)c(C)cc1Nc1ncc(Cl)c(Nc2ccc3nc(C)c(F)cc3c2P(C)(C)=O)n1] | Example 28 |
| Example 118 | 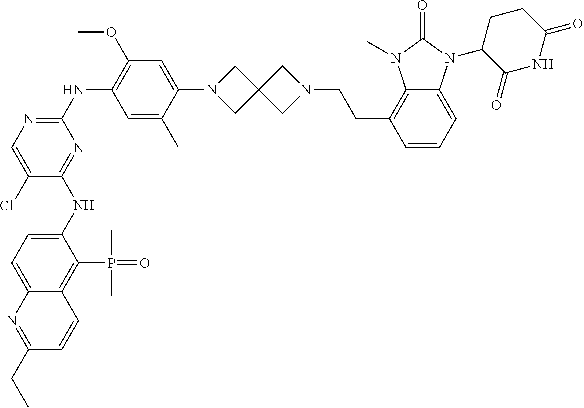 [SMILES: CCc1ccc2c(P(C)(C)=O)c(Nc3nc(Nc4cc(C)c(N5CC6(CN(CCc7cccc8c7n(C)c(=O)n8C7CCC(=O)NC7=O)C6)C5)cc4OC)ncc3Cl)ccc2n1] | Example 28 |
| Example 119 | 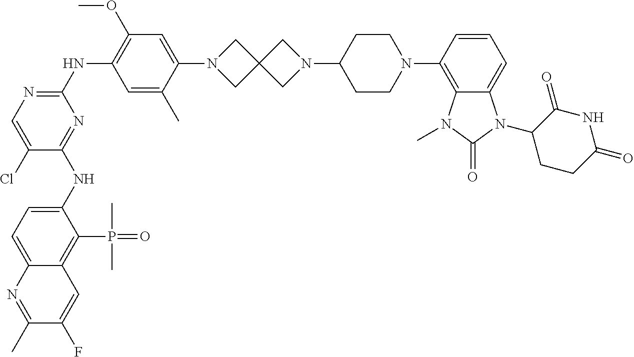 [SMILES: COc1cc(N2CC3(C2)CN(C2CCN(c4cccc5c4n(C)c(=O)n5C4CCC(=O)NC4=O)CC2)C3)c(C)cc1Nc1ncc(Cl)c(Nc2ccc3nc(C)c(F)cc3c2P(C)(C)=O)n1] | Example 21 |
| Example 120 | 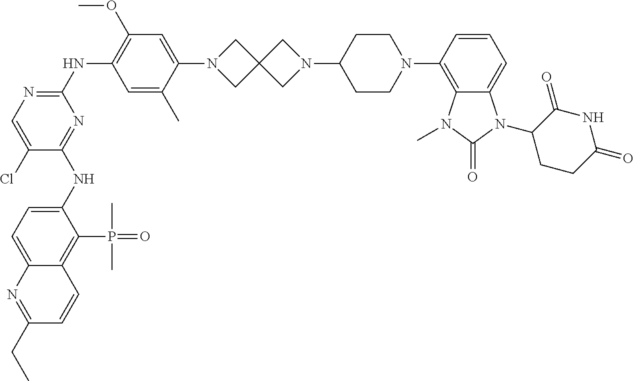 [SMILES: CCc1ccc2c(P(C)(C)=O)c(Nc3nc(Nc4cc(C)c(N5CC6(C5)CN(C5CCN(c7cccc8c7n(C)c(=O)n8C7CCC(=O)NC7=O)CC5)C6)cc4OC)ncc3Cl)ccc2n1] | Example 21 |
| Example 121 | 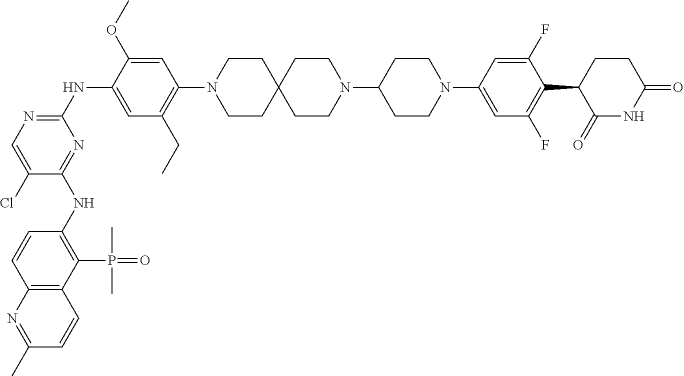 [SMILES: CCc1cc(Nc2ncc(Cl)c(Nc3ccc4nc(C)ccc4c3P(C)(C)=O)n2)c(OC)cc1N1CCC2(CC1)CCN(C1CCN(c3cc(F)c([C@H]4CCC(=O)NC4=O)c(F)c3)CC1)CC2] | Example 1 |
| Example 122 | 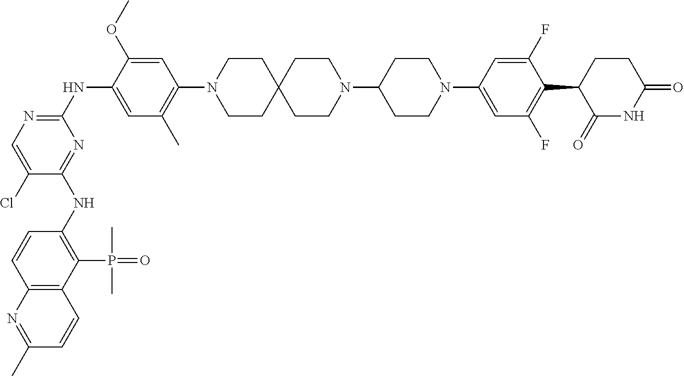 [SMILES: COc1cc(N2CCC3(CC2)CCN(C2CCN(c4cc(F)c([C@H]5CCC(=O)NC5=O)c(F)c4)CC2)CC3)c(C)cc1Nc1ncc(Cl)c(Nc2ccc3nc(C)ccc3c2P(C)(C)=O)n1] | Example 1 |
| Example 123 | 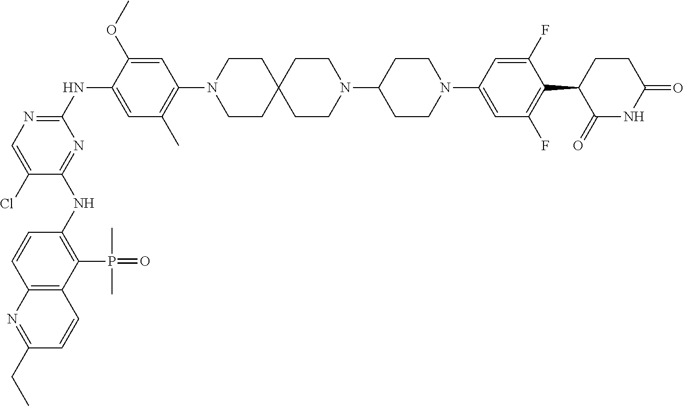 [SMILES: CCc1ccc2c(P(C)(C)=O)c(Nc3nc(Nc4cc(C)c(N5CCC6(CC5)CCN(C5CCN(c7cc(F)c([C@H]8CCC(=O)NC8=O)c(F)c7)CC5)CC6)cc4OC)ncc3Cl)ccc2n1] | Example 1 |
| Example 125 | 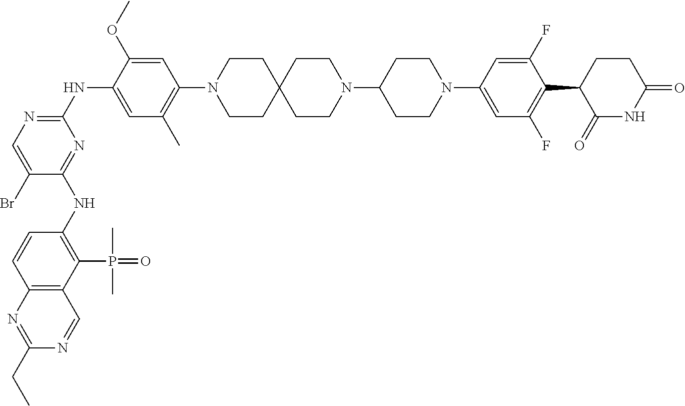 [SMILES: CCc1ncc2c(P(C)(C)=O)c(Nc3nc(Nc4cc(C)c(N5CCC6(CC5)CCN(C5CCN(c7cc(F)c([C@H]8CCC(=O)NC8=O)c(F)c7)CC5)CC6)cc4OC)ncc3Br)ccc2n1] | Example 1 |
| Example 126 | 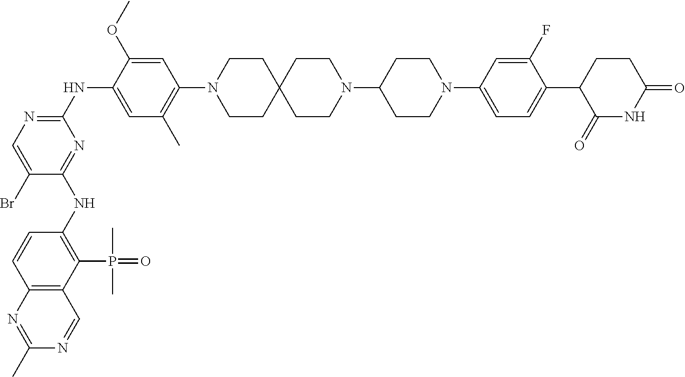 [SMILES: COc1cc(N2CCC3(CC2)CCN(C2CCN(c4ccc(C5CCC(=O)NC5=O)c(F)c4)CC2)CC3)c(C)cc1Nc1ncc(Br)c(Nc2ccc3nc(C)ncc3c2P(C)(C)=O)n1] | Example 1 |
| Example 127 | 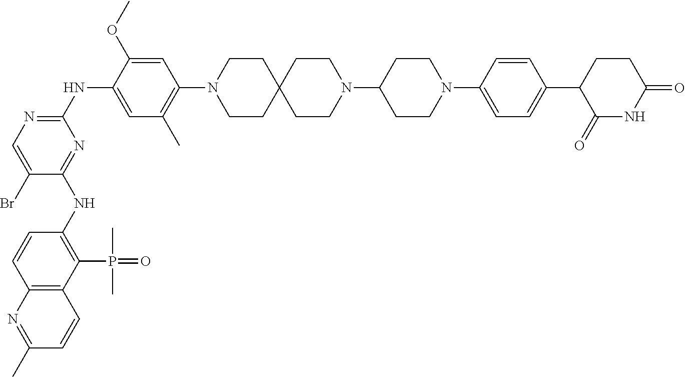 [SMILES: COc1cc(N2CCC3(CC2)CCN(C2CCN(c4ccc(C5CCC(=O)NC5=O)cc4)CC2)CC3)c(C)cc1Nc1ncc(Br)c(Nc2ccc3nc(C)ccc3c2P(C)(C)=O)n1] | Example 1 |
| Example 128 | 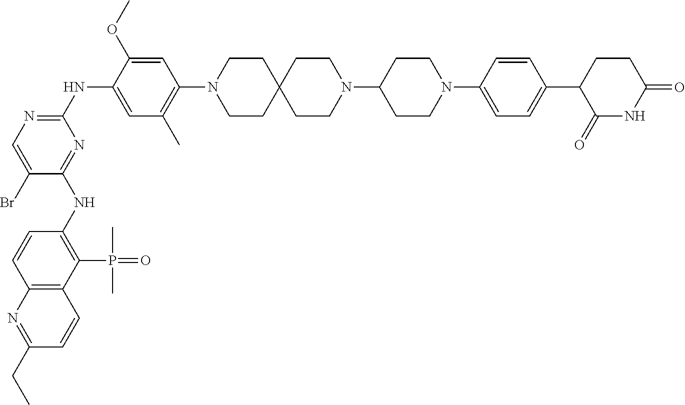 [SMILES: CCc1ccc2c(P(C)(C)=O)c(Nc3nc(Nc4cc(C)c(N5CCC6(CC5)CCN(C5CCN(c7ccc(C8CCC(=O)NC8=O)cc7)CC5)CC6)cc4OC)ncc3Br)ccc2n1] | Example 1 |
| Example 129 | 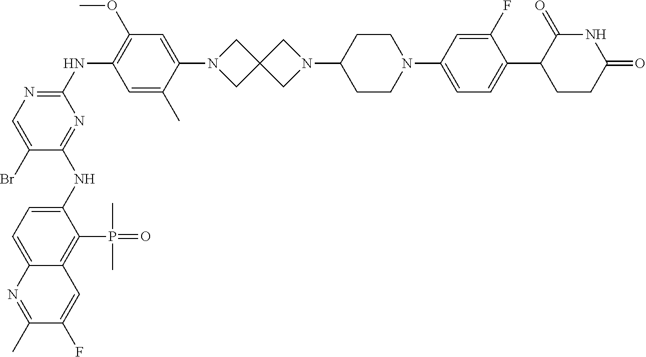 [SMILES: COc1cc(N2CC3(C2)CN(C2CCN(c4ccc(C5CCC(=O)NC5=O)c(F)c4)CC2)C3)c(C)cc1Nc1ncc(Br)c(Nc2ccc3nc(C)c(F)cc3c2P(C)(C)=O)n1] | Example 1 |
| Example 130 | 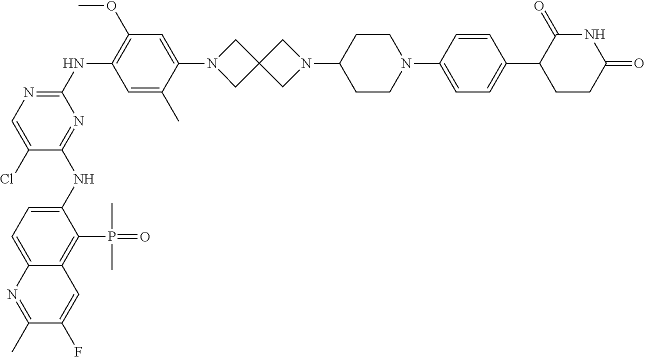 [SMILES: COc1cc(N2CC3(C2)CN(C2CCN(c4ccc(C5CCC(=O)NC5=O)cc4)CC2)C3)c(C)cc1Nc1ncc(Cl)c(Nc2ccc3nc(C)c(F)cc3c2P(C)(C)=O)n1] | Example 1 |
| Example 131 | 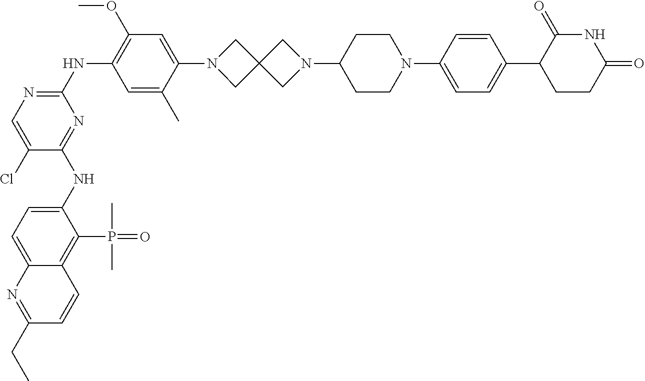 [SMILES: CCc1ccc2c(P(C)(C)=O)c(Nc3nc(Nc4cc(C)c(N5CC6(C5)CN(C5CCN(c7ccc(C8CCC(=O)NC8=O)cc7)CC5)C6)cc4OC)ncc3Cl)ccc2n1] | Example 1 |
| Example 132 | 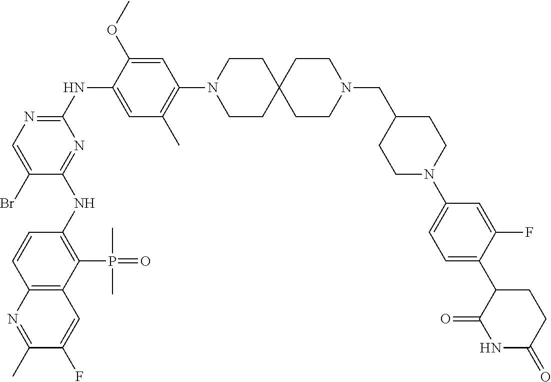 [SMILES: COc1cc(N2CCC3(CCN(CC4CCN(c5ccc(C6CCC(=O)NC6=O)c(F)c5)CC4)CC3)CC2)c(C)cc1Nc1ncc(Br)c(Nc2ccc3nc(C)c(F)cc3c2P(C)(C)=O)n1] | Example 1 |
| Example 133 | 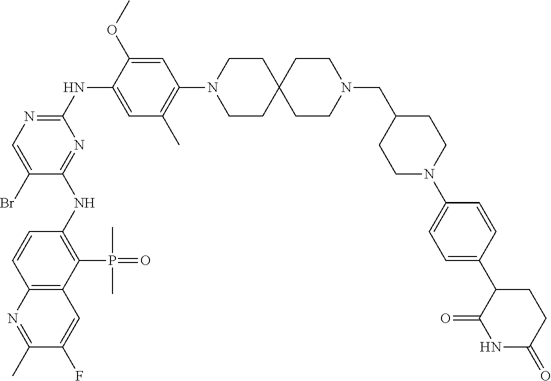 [SMILES: COc1cc(N2CCC3(CCN(CC4CCN(c5ccc(C6CCC(=O)NC6=O)cc5)CC4)CC3)CC2)c(C)cc1Nc1ncc(Br)c(Nc2ccc3nc(C)c(F)cc3c2P(C)(C)=O)n1] | Example 1 |
| Example 134 | 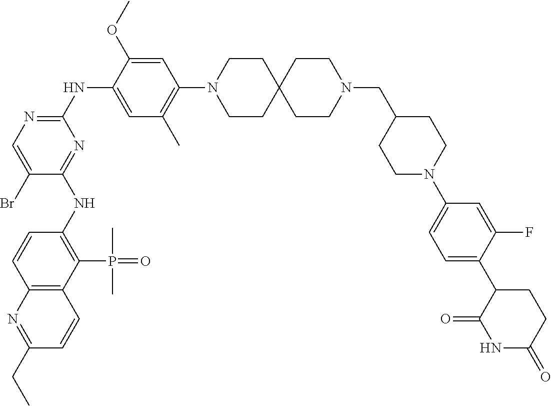 [SMILES: CCc1ccc2c(P(C)(C)=O)c(Nc3nc(Nc4cc(C)c(N5CCC6(CCN(CC7CCN(c8ccc(C9CCC(=O)NC9=O)c(F)c8)CC7)CC6)CC5)cc4OC)ncc3Br)ccc2n1] | Example 1 |
| Example 135 | 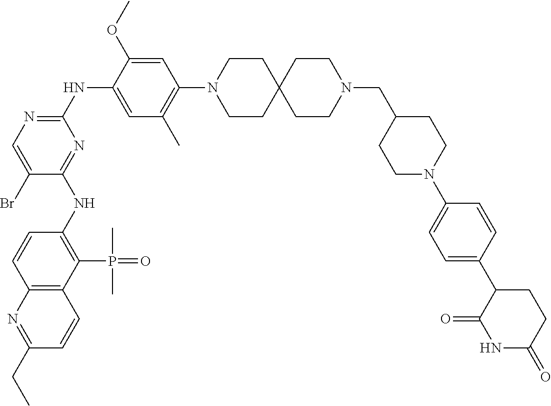 [SMILES: CCc1ccc2c(P(C)(C)=O)c(Nc3nc(Nc4cc(C)c(N5CCC6(CCN(CC7CCN(c8ccc(C9CCC(=O)NC9=O)cc8)CC7)CC6)CC5)cc4OC)ncc3Br)ccc2n1] | Example 1 |
| Example 136 | 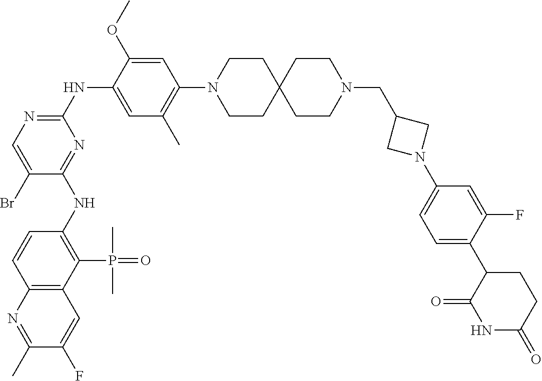 [SMILES: COc1cc(N2CCC3(CCN(CC4CN(c5ccc(C6CCC(=O)NC6=O)c(F)c5)C4)CC3)CC2)c(C)cc1Nc1ncc(Br)c(Nc2ccc3nc(C)c(F)cc3c2P(C)(C)=O)n1] | Example 1 |
| Example 137 | 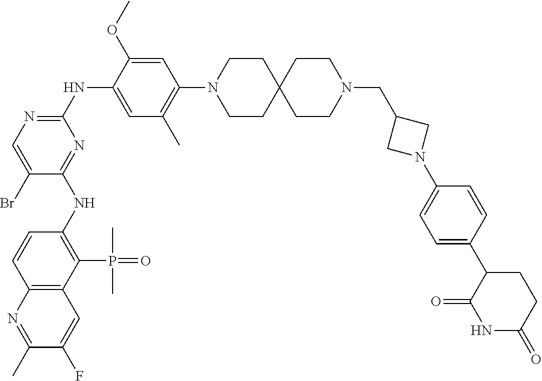 [SMILES: COc1cc(N2CCC3(CCN(CC4CN(c5ccc(C6CCC(=O)NC6=O)cc5)C4)CC3)CC2)c(C)cc1Nc1ncc(Br)c(Nc2ccc3nc(C)c(F)cc3c2P(C)(C)=O)n1] | Example 1 |
| Example 138 | 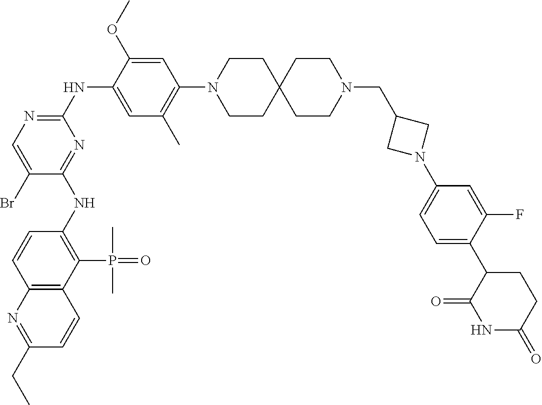 [SMILES: CCc1ccc2c(P(C)(C)=O)c(Nc3nc(Nc4cc(C)c(N5CCC6(CCN(CC7CN(c8ccc(C9CCC(=O)NC9=O)c(F)c8)C7)CC6)CC5)cc4OC)ncc3Br)ccc2n1] | Example 1 |
| Example 139 | 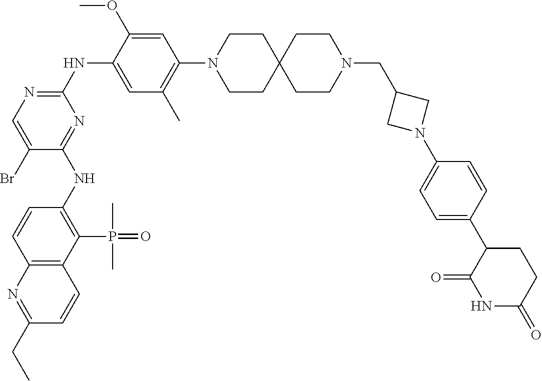 [SMILES: CCc1ccc2c(P(C)(C)=O)c(Nc3nc(Nc4cc(C)c(N5CCC6(CCN(CC7CN(c8ccc(C9CCC(=O)NC9=O)cc8)C7)CC6)CC5)cc4OC)ncc3Br)ccc2n1] | Example 1 |
| Example 140 | 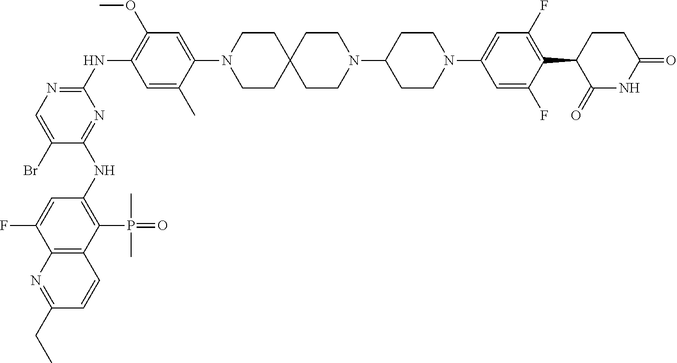 [SMILES: CCc1ccc2c(P(C)(C)=O)c(Nc3nc(Nc4cc(C)c(N5CCC6(CC5)CCN(C5CCN(c7cc(F)c([C@H]8CCC(=O)NC8=O)c(F)c7)CC5)CC6)cc4OC)ncc3Br)cc(F)c2n1] | Example 1 |
| Example 141 | 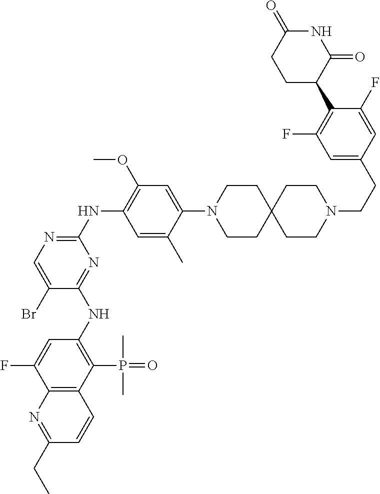 [SMILES: CCc1ccc2c(P(C)(C)=O)c(Nc3nc(Nc4cc(C)c(N5CCC6(CCN(CCc7cc(F)c([C@H]8CCC(=O)NC8=O)c(F)c7)CC6)CC5)cc4OC)ncc3Br)cc(F)c2n1] | Example 1 |
| Example 142 | 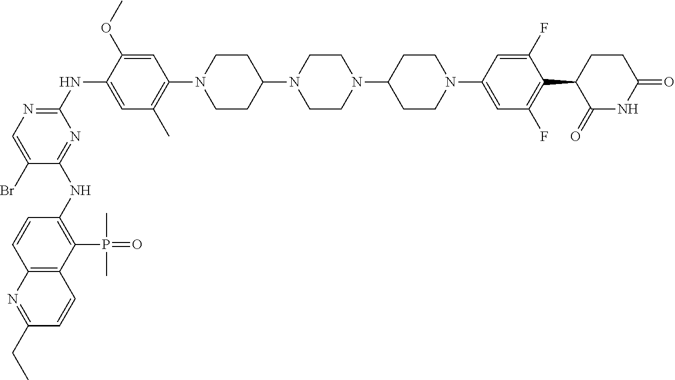 [SMILES: CCc1ccc2c(P(C)(C)=O)c(Nc3nc(Nc4cc(C)c(N5CCC(N6CCN(C7CCN(c8cc(F)c([C@H]9CCC(=O)NC9=O)c(F)c8)CC7)CC6)CC5)cc4OC)ncc3Br)ccc2n1] | Example 1 |
| Example 143 | 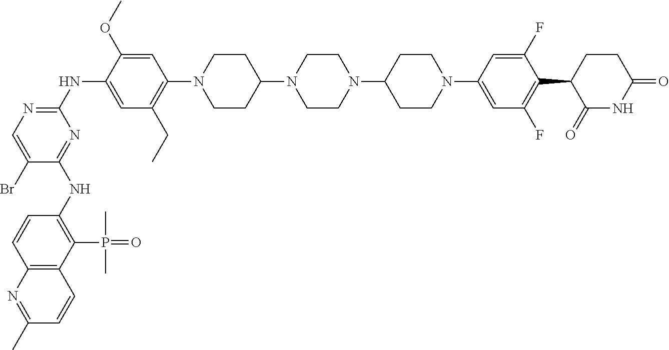 [SMILES: CCc1cc(Nc2ncc(Br)c(Nc3ccc4nc(C)ccc4c3P(C)(C)=O)n2)c(OC)cc1N1CCC(N2CCN(C3CCN(c4cc(F)c([C@H]5CCC(=O)NC5=O)c(F)c4)CC3)CC2)CC1] | Example 1 |

### Cell Line Generation

H1975-clone #28(Del19/T790M/C797S) and H1975-clone #25(L858R/T790M/C797S). EGFR-Del19/T790M/C797S and EGFR-L858R/T790M/C797S were stably expressed in H1975 cell lines by lentivirus-mediated over-expression, respectively. The EGFR over-expressed cells then underwent knockout, in which the EGFR targeting sgRNA was designed to only target the endogenous EGFR copies and preserve the exogenous EGFR copies. Followed by knockout, the edited H1975 cells were seeded in 96 well plates at the concentration of 1 cell/cell, cultured for about 2 weeks to allow single clones formation. The formed clones were screened by DNA sequencing and whole exon sequencing analysis for the desired edition.

H1975-clone #28, and H1975-clone #25 were finally confirmed as homozygous Del19/T790M/C797S EGFR, and L858R/T790M/C797S EGFR clones, respectively.

### Cell Degradation

### Cell Treatment

On day 1, H1975-clone #28(Del19/T790M/C797S), and H1975-clone #25(L858R/T790M/C797S) cells are seeded at 20000 cells/well, 30000 cells/well, 10000 cells/well or 5000 cells/well correspondingly in cell culture medium [RPMI1640 (Gibco, Cat #72400-047), 10% heat-inactive FBS, 1% PS (Gibco, Cat #10378)] in Corning 96 well plate (Cat #3599).

H1975-#25 and H1975-#28 cells are treated with compounds diluted in 0.2% DMSO cell culture medium on day 2, incubate for 16 h, 37° C., 5% CO₂. the final concentration of compounds in all assay is start with 10 uM, 5-fold dilution, total 8 doses were included.

### HTRF Assay

After 16 h treatment, add HTRF lysis buffer to each well; seal the plate and incubate 1 hour at room temperature on a plate shaker; Once the cells are lysed, 16 μL of cell lysate are transferred to a PE 384-well HTRF detection plate; 4 μL of pre-mixed HTRF antibodies are added to each well; Cover the plate with a plate sealer, spin 1000 rpm for 1 min, Incubate overnight at room temperature; Read on BMG PheraStar with HTRF protocol (337 nm-665 nm-620 nm).

The inhibition (degradation) percentage of the compound was calculated by the following equation:

Inhibition percentage of Compound=100−100×(Signal−low control)/(High control−low control),

wherein signal=each test compound group
Low control=only lysis buffer without cells, indicating that EGFR is completely degraded;
High control=Cell group with added DMSO and without compound, indicating microplate readings without EGFR degradation;
Dmax is the maximum percentage of inhibition (degradation).

The IC₅₀ (DC₅₀) value of a compound can be obtained by fitting the following equation

$$\\ Y = Bottom + ( TOP ‐ Bottom ) / ( 1 + ( ( {IC}_{50} / X ) {}_{} {\hat{}}_{} hillslope ) )$$

Wherein, X and Y are known values, and IC₅₀, Hillslope, Top and Bottom are the parameters obtained by fitting with software. Y is the inhibition percentage (calculated from the equation), X is the concentration of the compound; IC₅₀ is the concentration of the compound when the 50% inhibition is reached. The smaller the IC₅₀ value is, the stronger the inhibitory ability of the compound is. Vice versa, the higher the IC₅₀ value is, the weaker the ability the inhibitory ability of the compound is; Hillslope represents the slope of the fitted curve, generally around 1 *; Bottom represents the minimum value of the curve obtained by data fitting, which is generally 0%±20%; Top represents the maximum value of the curve obtained by data fitting, which is generally 100%±20%. The experimental data were fitted by calculating and analyzing with Dotmatics data analysis software.

TABLE 1
Degradation (H1975 # 28 DTC and H1975 #
25-LTC) result for Example 5 to Example 107

| | |
|---|---|
| H1975 # 28 DTC | H1975 # 25-LTC |

| Example | DC₅₀(nM) | Dmax (%) | DC₅₀(nM) | Dmax (%) |
|---|---|---|---|---|
| 1 | 2.2 | 89 | 3.4 | 92 |
| 2 | 3.5 | 89 | 3.4 | 92 |
| 3 | 1.9 | 91 | 2.8 | 95 |
| 4 | 1.5 | 87 | 2.5 | 84 |
| 5 | 3.1 | 90 | 3.7 | 92 |
| 6 | 3.1 | 80 | 9.0 | 89 |
| 7 | 2.7 | 86 | 2.9 | 76 |
| 8 | 0.6 | 80 | 1.6 | 87 |
| 9 | 1.3 | 86 | 3.8 | 86 |
| 10 | 2.7 | 88 | 4.9 | 91 |
| 11 | 1.8 | 88 | 0.9 | 94 |
| 12 | 2.9 | 91 | 2.5 | 92 |
| 13 | 0.7 | 92 | 1.8 | 94 |
| 14 | 1.9 | 89 | 2.1 | 91 |
| 15 | 2.7 | 95 | 3.3 | 94 |
| 16 | 2.8 | 88 | 1.1 | 85 |
| 17 | 1.7 | 92 | 2.9 | 87 |
| 18 | 1.7 | 91 | 1.7 | 92 |
| 19 | 1.1 | 91 | 1.9 | 93 |
| 20 | 2.9 | 90 | 1.4 | 92 |
| 21 | 0.6 | 77 | 1.8 | 78 |
| 22 | 0.6 | 74 | 0.9 | 83 |
| 23 | 0.6 | 74 | 0.9 | 80 |
| 24 | 0.6 | 87 | 1.4 | 88 |
| 25 | 0.8 | 74 | 0.5 | 82 |
| 26 | 1.4 | 75 | 0.7 | 80 |
| 27 | 1.3 | 91 | 2.4 | 92 |
| 28 | 1.0 | 80 | 2.2 | 87 |
| 29 | 3.1 | 88 | 3.8 | 96 |
| 30 | 13.3 | 87 | 10.8 | 89 |
| 31 | 0.8 | 80 | 0.6 | 86 |
| 32 | 1.4 | 77 | 1.3 | 81 |
| 33 | 2.1 | 82 | 1.5 | 91 |
| 34 | 2.5 | 80 | 2.8 | 89 |
| 35 | 1.2 | 79 | 1.3 | 83 |
| 36 | 3.1 | 93 | 4.5 | 94 |
| 37 | 3.3 | 90 | 1.4 | 95 |
| 38 | 7.4 | 85 | 8.6 | 89 |
| 39 | 2.6 | 92 | 5.6 | 93 |
| 40 | 2.4 | 88 | 6.0 | 86 |
| 41 | 4.2 | 86 | 4.2 | 88 |
| 42 | 1.5 | 93 | 4.9 | 85 |
| 43 | 3.7 | 87 | 4.6 | 90 |
| 44 | 3.5 | 90 | 4.2 | 92 |
| 45 | 7.7 | 81 | 7.4 | 86 |
| 46 | 3.3 | 80 | 5.4 | 89 |
| 47 | 14.3 | 73 | 16.0 | 88 |
| 48 | 1.7 | 83 | 2.8 | 90 |
| 49 | 3.8 | 82 | 7.6 | 89 |
| 51 | 2.5 | 77 | 3.1 | 89 |
| 52 | 3.4 | 79 | 3.8 | 90 |
| 53 | 3.0 | 93 | 4.0 | 93 |
| 54 | 3.6 | 86 | 3.6 | 88 |
| 55 | 5.6 | 91 | 2.4 | 92 |
| 56 | 8.2 | 86 | 5.6 | 90 |
| 57 | 4.5 | 88 | 2.7 | 90 |
| 58 | 1.6 | 91 | 3.1 | 91 |
| 59 | 3.8 | 87 | 2.7 | 92 |
| 60 | 0.5 | 95 | 1.1 | 95 |
| 61 | 0.9 | 94 | 1.2 | 94 |
| 62 | 1.4 | 89 | 1.6 | 95 |
| 63 | 4.7 | 74 | 2.7 | 77 |
| 64 | 1.1 | 94 | 1.5 | 95 |
| 66 | 3.5 | 91 | 6.9 | 93 |
| 68 | 2.2 | 82 | 3.1 | 81 |
| 72 | 1.8 | 82 | 2.0 | 82 |
| 78 | 1.9 | 90 | 5.4 | 74 |
| 79 | 6.6 | 90 | 3.3 | 95 |
| 92 | 3.3 | 67 | 8.2 | 85 |

The foregoing examples and description of certain embodiments should be taken as illustrating, rather than as limiting the present invention as defined by the claims. As will be readily appreciated, numerous variations and combinations of the features set forth above can be utilized without departing from the present invention as set forth in the claims. All such variations are intended to be included within the scope of the present invention. All references cited are incorporated herein by reference in their entireties.

It is to be understood that, if any prior art publication is referred to herein, such reference does not constitute an admission that the publication forms a part of the common general knowledge in the art in any country.

What is claimed is:

1. A compound of Formula (I): 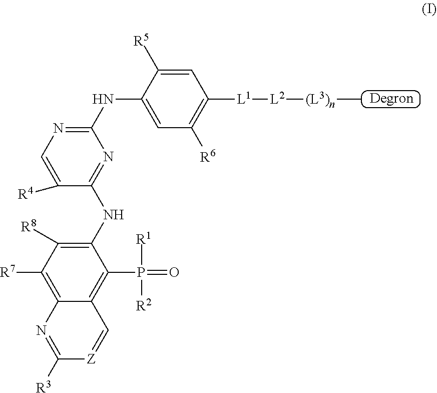
or a N-oxide thereof, or a pharmaceutically acceptable salt thereof, or a stereoisomer thereof, or a deuterated analog thereof, or a prodrug thereof,
wherein:
Z is N or CR^{z};
R^{z} is H, —C₁-C₈alkyl, C₃-C₈cycloalkyl or halogen;
R¹ and R² are each independently —C₁₋₈alkyl or —C₃₋₈cycloalkyl; each of said —C₁-C₈alkyl or C₃-C₈cycloalkyl is optionally substituted with at least one halogen;
R³ is hydrogen, —C₁-C₈alkyl, —C₂-C₈alkenyl, —C₂-C₈alkynyl, C₃-C₈cycloalkyl, halogen, 3- to 8-membered heterocyclyl, C₆-C₁₂aryl, 5- to 12-membered heteroaryl, —OR^{3a}, —SR^{3a}, —CN, —C(O)R^{3a}, —CO₂R^{3a}, —C(O)NR^{3a}R^{3b}, —NR^{3a}R^{3b}, —NR^{3a}COR^{3b}; each of said —C₁-C₈alkyl, —C₂-C₈alkenyl, —C₂-C₈alkynyl, C₃-C₈cycloalkyl, 3- to 8-membered heterocyclyl, C₆-C₁₂aryl or 5- to 12-membered heteroaryl is optionally substituted with 0 to 2 R^{3c};
R^{3a} and R^{3b} are each independently selected from hydrogen, —C₁-C₈alkyl, —C₁-C₈haloalkyl, —C₂-C₈alkenyl, —C₂-C₈alkynyl, C₁-C₈alkoxy-C₁-C₈alkyl-, C₃-C₈cycloalkyl, 3- to 8-membered heterocyclyl, C₆-C₁₂aryl or 5- to 12-membered heteroaryl;
R^{3c}, at each occurrence, is independently halogen, —OH, —CN, oxo (═O), —C₁-C₈alkyl, —C₂-C₈alkenyl, —C₂-C₈alkynyl, —C₃-C₈cycloalkyl, 3- to 8-membered heterocyclyl, —C₆-C₁₂aryl, or 5- to 12-membered heteroaryl;
R⁴ is halogen, methyl or methoxy;
R⁵ is hydrogen, halogen, —C₁-C₈alkyl, —NR^{5a}R^{5b}, —OR^{5a}, —SR^{5a}, C₃-C₈cycloalkyl, 3- to 8-membered heterocyclyl, C₆-C₁₂aryl, 5- to 12-membered heteroaryl, or CN; each of —C₁-C₈alkyl, C₃-C₈cycloalkyl, 3- to 8-membered heterocyclyl, C₆-C₁₂aryl, or 5- to 12-membered heteroaryl is optionally substituted with at least one R^{5c};
R^{5a} and R^{5b} are each independently selected from hydrogen, —C₁-C₈alkyl, —C₂-C₈alkenyl, —C₂-C₈alkynyl, C₃-C₈cycloalkyl, 3- to 8-membered heterocyclyl, C₆-C₁₂aryl, or 5- to 12-membered heteroaryl, each of said —C₁-C₈alkyl, —C₂-C₈alkenyl, —C₂-C₈alkynyl, C₃-C₈cycloalkyl, 3- to 8-membered heterocyclyl, C₆-C₁₂aryl, or 5- to 12-membered heteroaryl is optionally substituted with at least one substituent R^{7d};
R^{5c} and R^{5d} are each independently selected from halogen, hydroxy, —C₁-C₈alkyl, —C₁-C₈alkoxy, —C₂—C₈alkenyl, —C₂-C₈alkynyl, C₃-C₈cycloalkyl, 3- to 8-membered heterocyclyl, C₆-C₁₂aryl, or 5- to 12-membered heteroaryl;
R⁶ is hydrogen, halogen, —C₁-C₈alkyl, —NR^{6a}R^{6b}, —OR^{6a}, —SR^{6a}, C₃-C₈cycloalkyl, 3- to 8-membered heterocyclyl, C₆-C₁₂aryl, 5- to 12-membered heteroaryl, or CN; each of —C₁-C₈alkyl, C₃-C₈cycloalkyl, 3- to 8-membered heterocyclyl, C₆-C₁₂aryl, or 5- to 12-membered heteroaryl is optionally substituted with at least one R^{6c};
R^{6a} and R^{6b} are each independently selected from hydrogen, —C₁-C₈alkyl, —C₂-C₈alkenyl, —C₂-C₈alkynyl, C₃-C₈cycloalkyl, 3- to 8-membered heterocyclyl, C₆-C₁₂aryl, or 5- to 12-membered heteroaryl, each of said —C₁-C₈alkyl, —C₂-C₈alkenyl, —C₂-C₈alkynyl, C₃-C₈cycloalkyl, 3- to 8-membered heterocyclyl, C₆-C₁₂aryl, or 5- to 12-membered heteroaryl is optionally substituted with at least one substituent R^{6d};
R^{6c} and R^{6d} are each independently selected from halogen, hydroxy, —C₁-C₈alkyl, —C₁-C₈alkoxy, —C₂-C₈alkenyl, —C₂-C₈alkynyl, C₃-C₈cycloalkyl, 3- to 8-membered heterocyclyl, C₆-C₁₂aryl, or 5- to 12-membered heteroaryl;
R⁷ and R⁸ are each independently selected from hydrogen, halogen, —C₁-C₈alkyl, —NR^{7a}R^{7b}, —OR^{7a}, —C₃-C₈cycloalkyl, 3- to 8-membered heterocyclyl, —C₆-C₁₂aryl, 5- to 12-membered heteroaryl, or —CN; each of —C₁-C₈alkyl, C₃-C₈cycloalkyl, 3- to 8-membered heterocyclyl, C₆-C₁₂aryl or 5- to 12-membered heteroaryl is optionally substituted with at least one substituent R^{7c}; or
R^{7a} and R^{7b} are each independently selected from hydrogen, —C₁-C₈alkyl, —C₂-C₈alkenyl, —C₂-C₈alkynyl, —C₃-C₈cycloalkyl, 3- to 8-membered heterocyclyl, —C₆-C₁₂aryl or 5- to 12-membered heteroaryl; each of said —C₁-C₈alkyl, —C₂-C₈alkenyl, —C₂-C₈alkynyl, C₃-C₈cycloalkyl, 3- to 8-membered heterocyclyl, C₆-C₁₂aryl or 5- to 12-membered heteroaryl is optionally substituted with at least one substituent R^{7d};
R^{7c} and R^{7d} are each independently halogen, hydroxy, —C₁-C₈alkyl, —C₁-C₈alkoxy, —C₂-C₈alkenyl, —C₂-C₈alkynyl, —C₃-C₈cycloalkyl, 3- to 8-membered heterocyclyl, —C₆-C₁₂aryl or 5- to 12-membered heteroaryl; 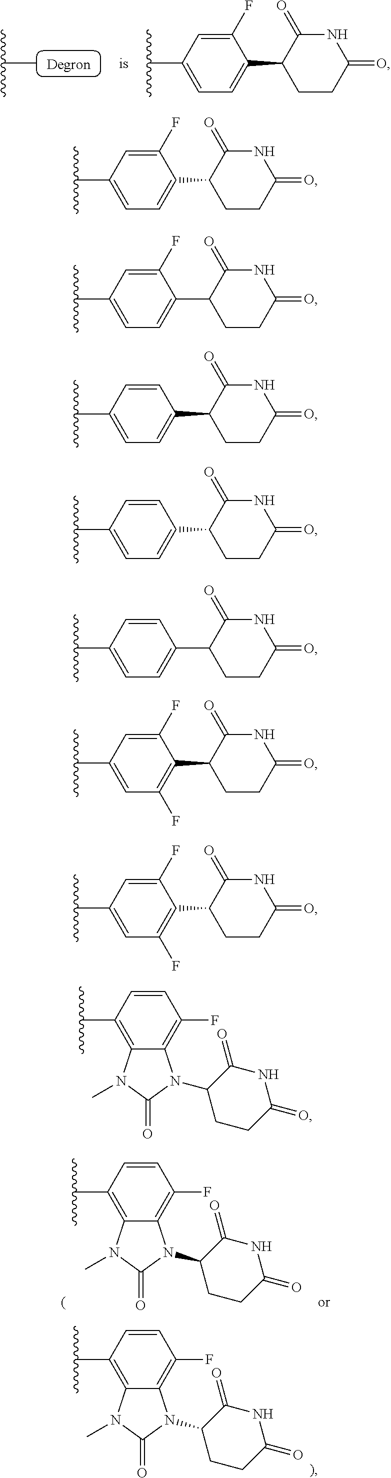 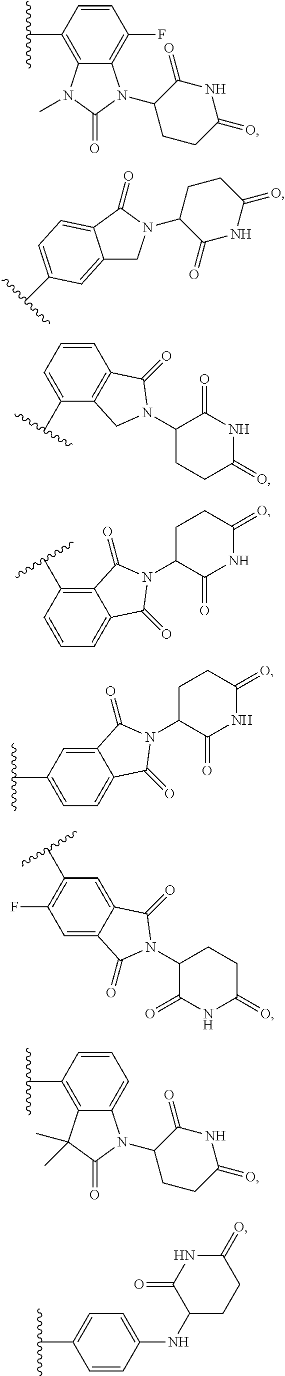 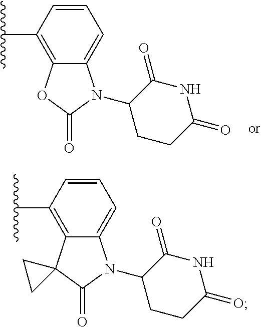
L¹ is 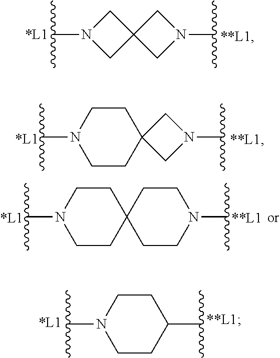
wherein * refers to the position attached to the 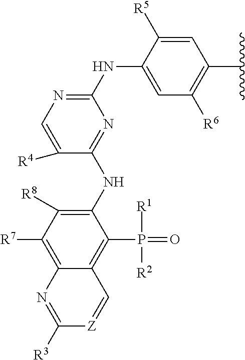
moiety, and **^{L1} refers to the position attached to the 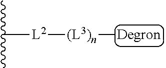
moiety;
L² is 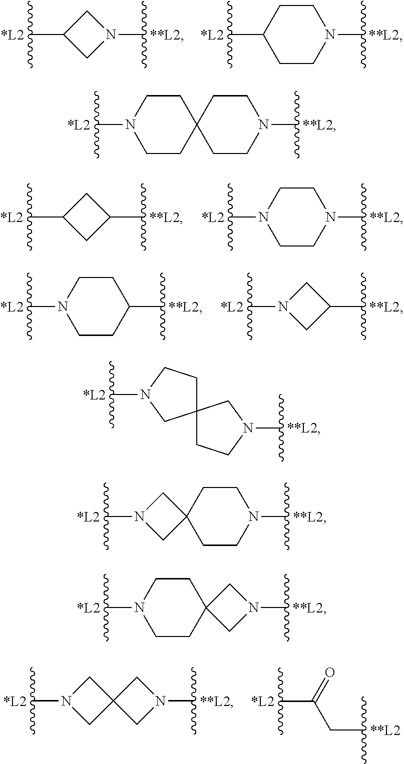
or *^{L2}-(CH₂)₁₋₃—**^{L2}; wherein *^{L2} refers to the position attached to the 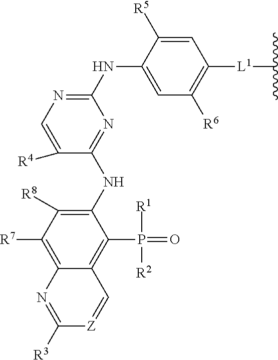
moiety, and **^{L2} refers to the position attached to the 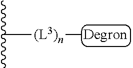
moiety;
L³ is 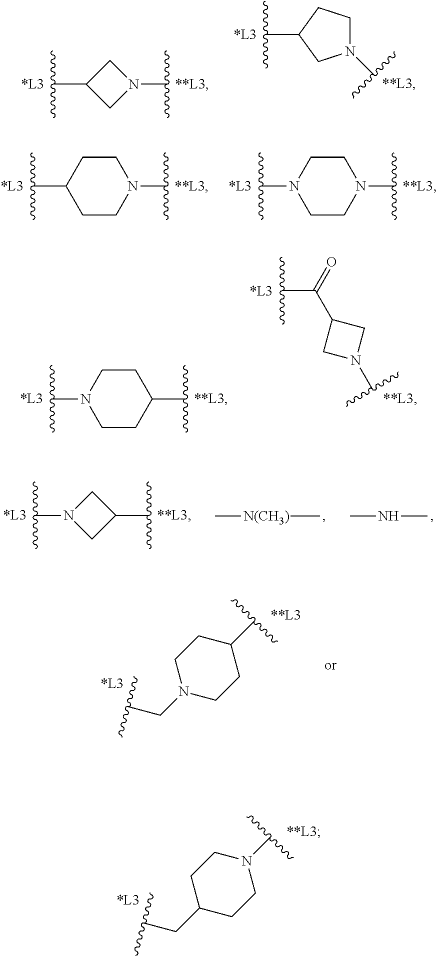
wherein *^{L3} refers to the position attached to the 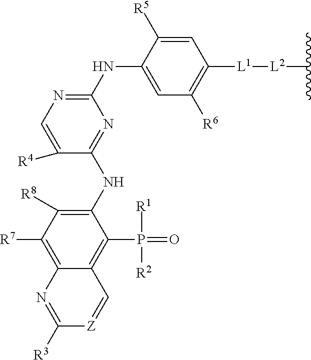
moiety, and **^{L3} refers to the position attached to the 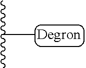
moiety;
each of said 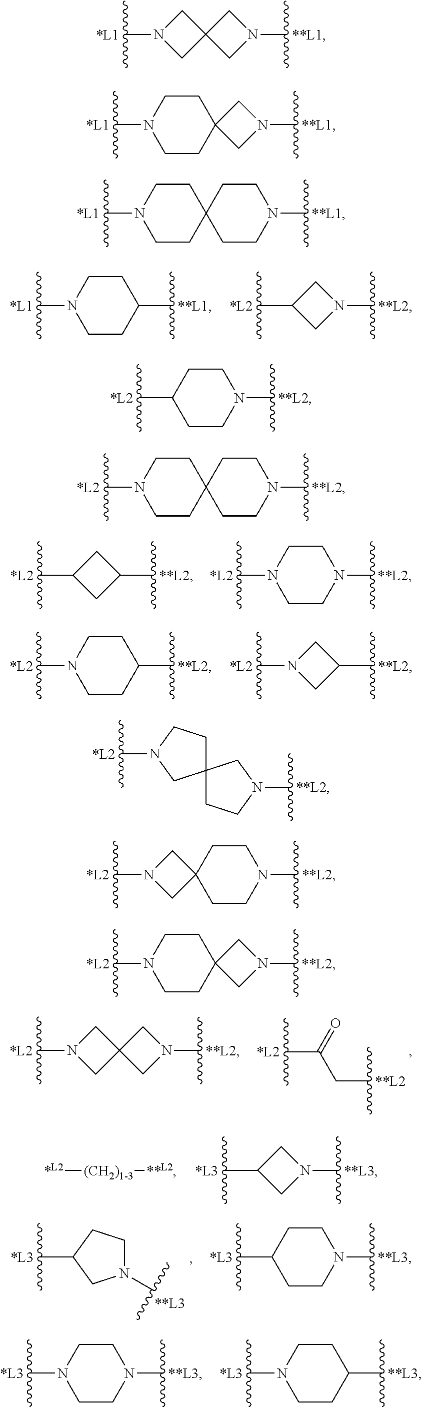 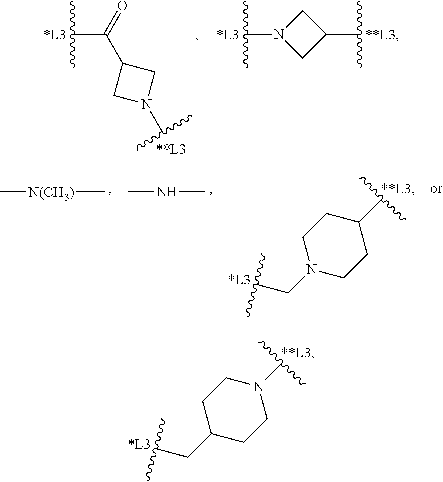
or is optionally substituted with 0 to 2 R^{La};
R^{La}, at each occurrence, is independently halogen, —OH, —CN, oxo (═O), —C₁-C₈alkyl, —C₂-C₈alkenyl, —C₂-C₈alkynyl, —C₃-C₈cycloalkyl, 3- to 8-membered heterocyclyl, —C₆-C₁₂aryl, or 5- to 12-membered heteroaryl;
n is 0 or 1;
provided that the compound is not

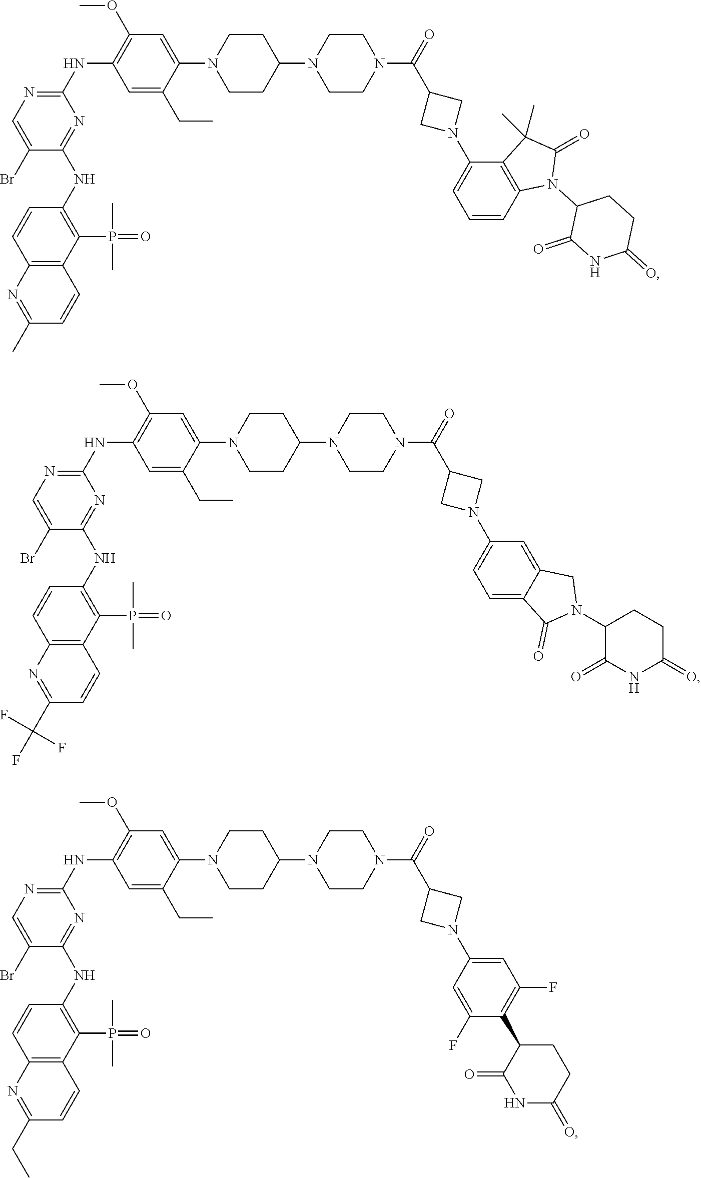 [SMILES: CCc1cc(Nc2ncc(Br)c(Nc3ccc4nc(C)ccc4c3P(C)(C)=O)n2)c(OC)cc1N1CCC(N2CCN(C(=O)C3CN(c4cccc5c4C(C)(C)C(=O)N5C4CCC(=O)NC4=O)C3)CC2)CC1 ; CCc1cc(Nc2ncc(Br)c(Nc3ccc4nc(C(F)(F)F)ccc4c3P(C)(C)=O)n2)c(OC)cc1N1CCC(N2CCN(C(=O)C3CN(c4ccc5c(c4)CN(C4CCC(=O)NC4=O)C5=O)C3)CC2)CC1 ; CCc1ccc2c(P(C)(C)=O)c(Nc3nc(Nc4cc(CC)c(N5CCC(N6CCN(C(=O)C7CN(c8cc(F)c([C@H]9CCC(=O)NC9=O)c(F)c8)C7)CC6)CC5)cc4OC)ncc3Br)ccc2n1]

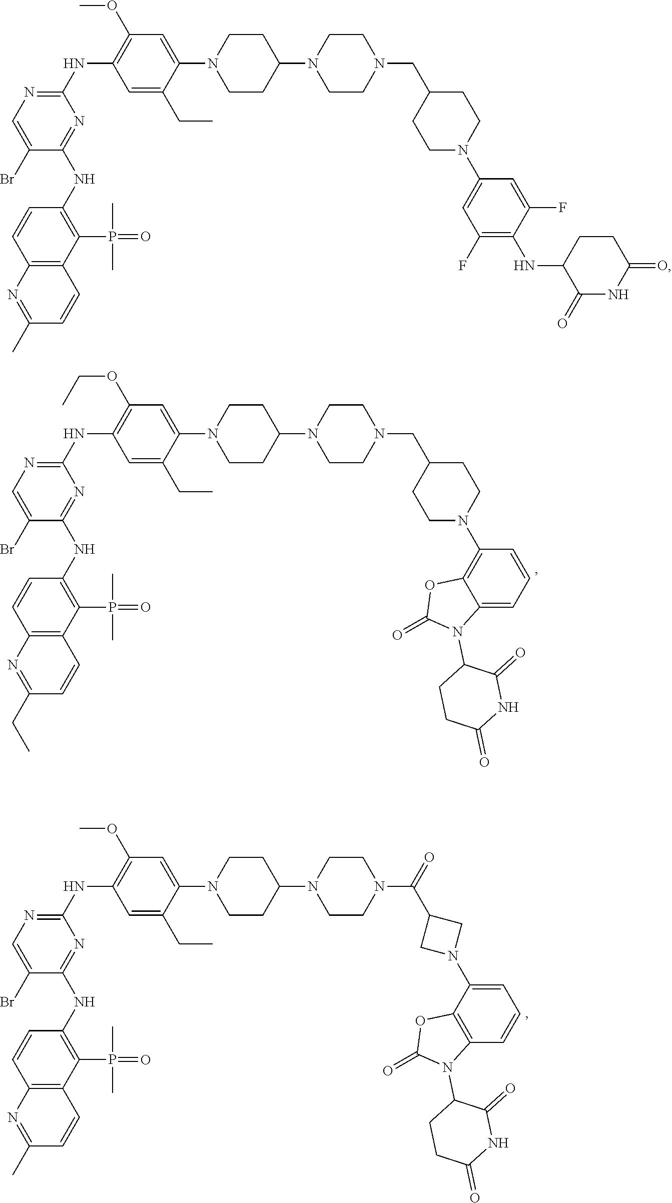 [SMILES: CCc1cc(Nc2ncc(Br)c(Nc3ccc4nc(C)ccc4c3P(C)(C)=O)n2)c(OC)cc1N1CCC(N2CCN(CC3CCN(c4cc(F)c(NC5CCC(=O)NC5=O)c(F)c4)CC3)CC2)CC1 ; CCOc1cc(N2CCC(N3CCN(CC4CCN(c5cccc6c5oc(=O)n6C5CCC(=O)NC5=O)CC4)CC3)CC2)c(CC)cc1Nc1ncc(Br)c(Nc2ccc3nc(CC)ccc3c2P(C)(C)=O)n1 ; CCc1cc(Nc2ncc(Br)c(Nc3ccc4nc(C)ccc4c3P(C)(C)=O)n2)c(OC)cc1N1CCC(N2CCN(C(=O)C3CN(c4cccc5c4oc(=O)n5C4CCC(=O)NC4=O)C3)CC2)CC1]

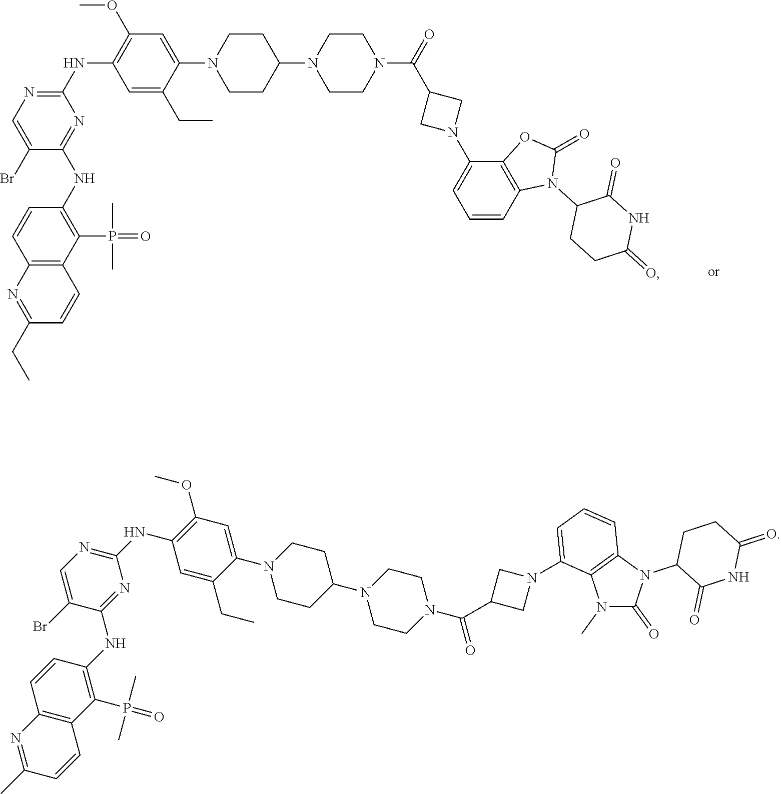 [SMILES: CCc1ccc2c(P(C)(C)=O)c(Nc3nc(Nc4cc(CC)c(N5CCC(N6CCN(C(=O)C7CN(c8cccc9c8oc(=O)n9C8CCC(=O)NC8=O)C7)CC6)CC5)cc4OC)ncc3Br)ccc2n1 ; "or" ; CCc1cc(Nc2ncc(Br)c(Nc3ccc4nc(C)ccc4c3P(C)(C)=O)n2)c(OC)cc1N1CCC(N2CCN(C(=O)C3CN(c4cccc5c4n(C)c(=O)n5C4CCC(=O)NC4=O)C3)CC2)CC1]

2. The compound of claim 1, wherein the compound is formula (IIa) or (IIb) 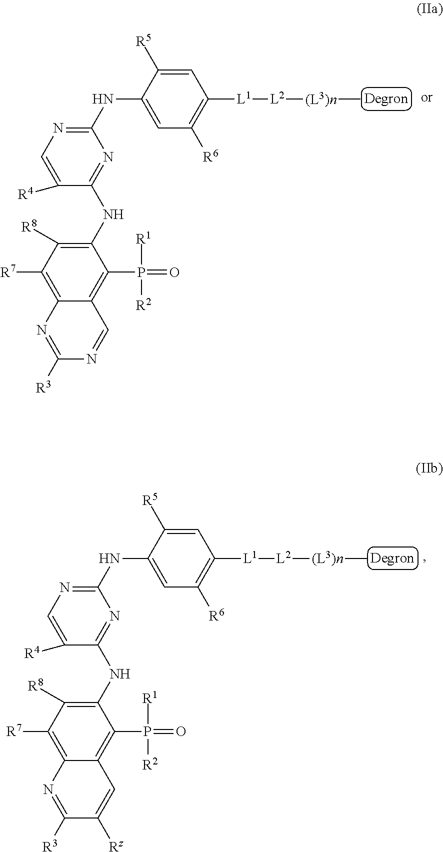
wherein
R^{z}, R¹, R², R³, R⁴, R⁵, R⁶, R⁷, R⁸, 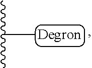
L¹, L², L^{Z}, L³ and n are each defined as claim 1.

3. The compound of claim 1, wherein the compound is formula (IIIa) to (IIIu) 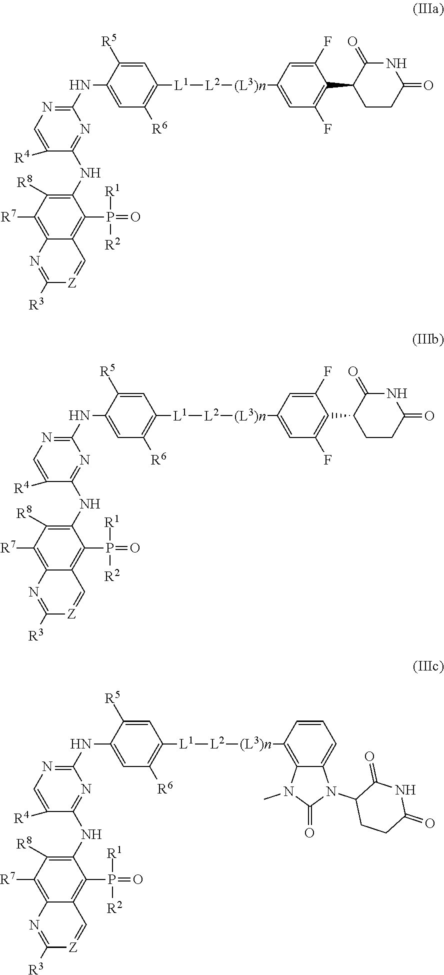 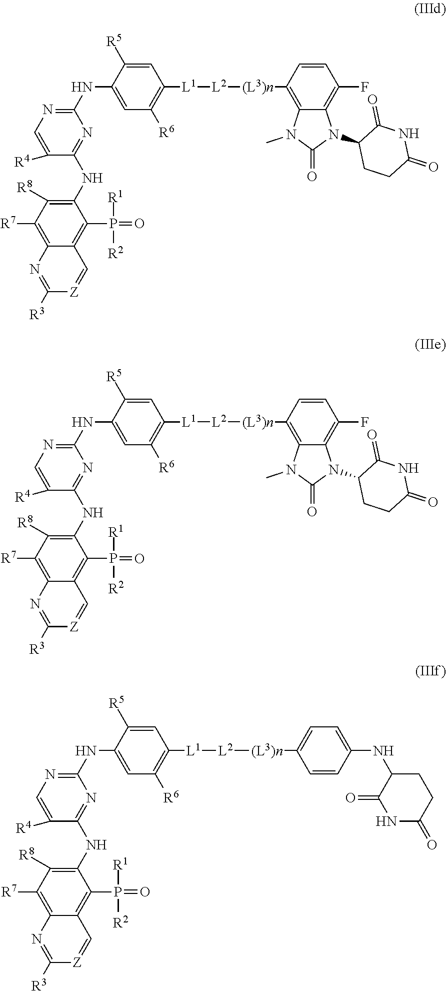 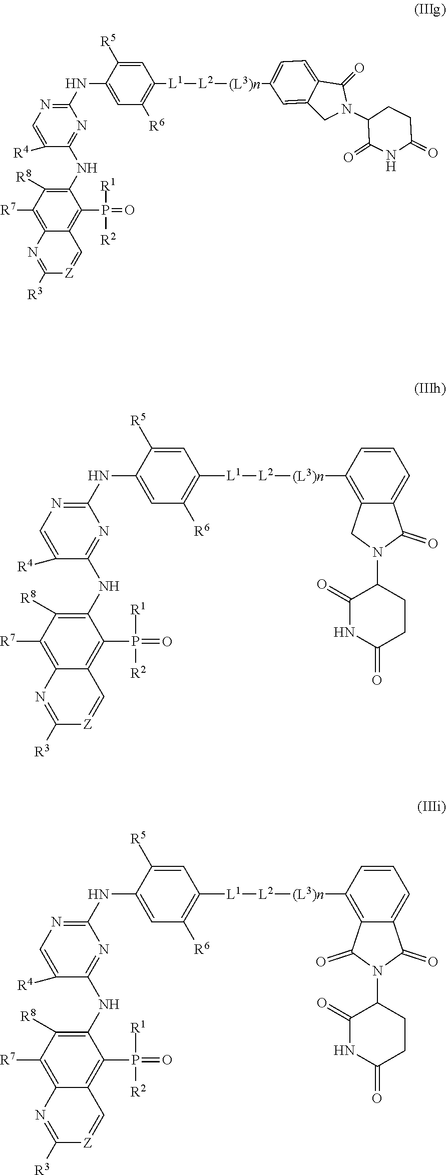 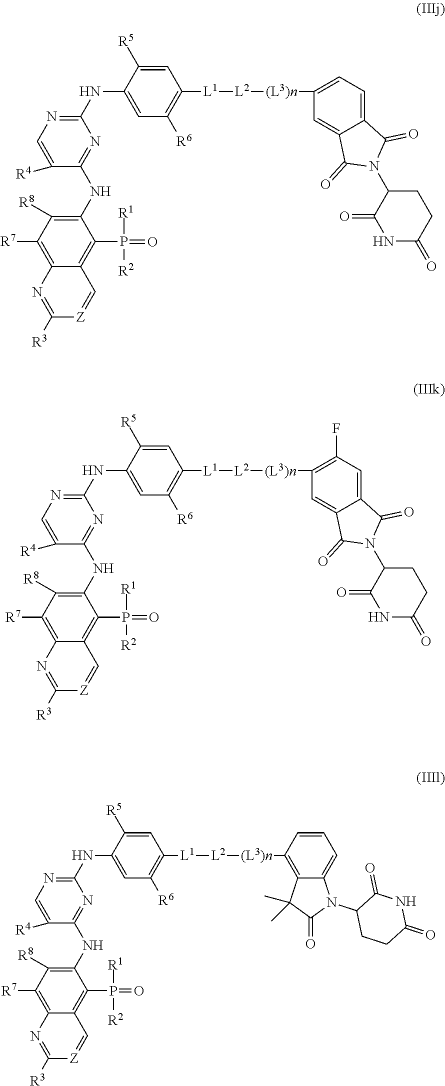 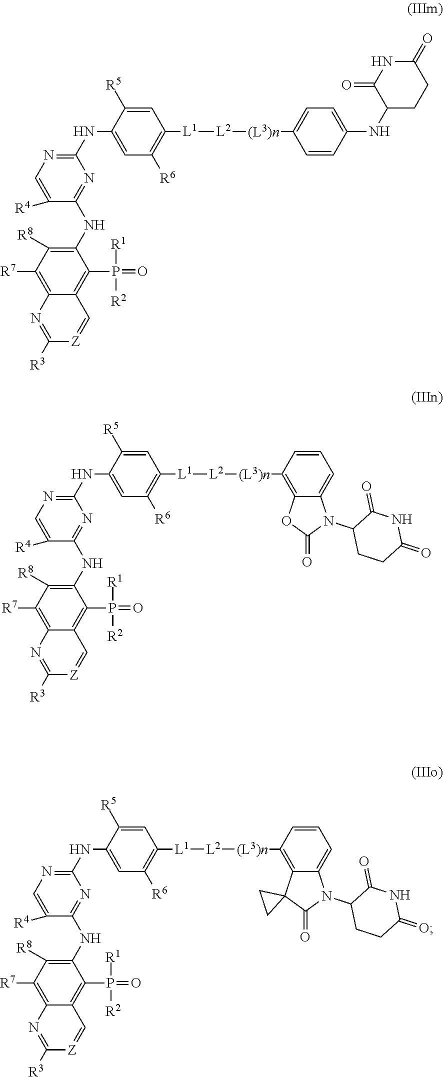 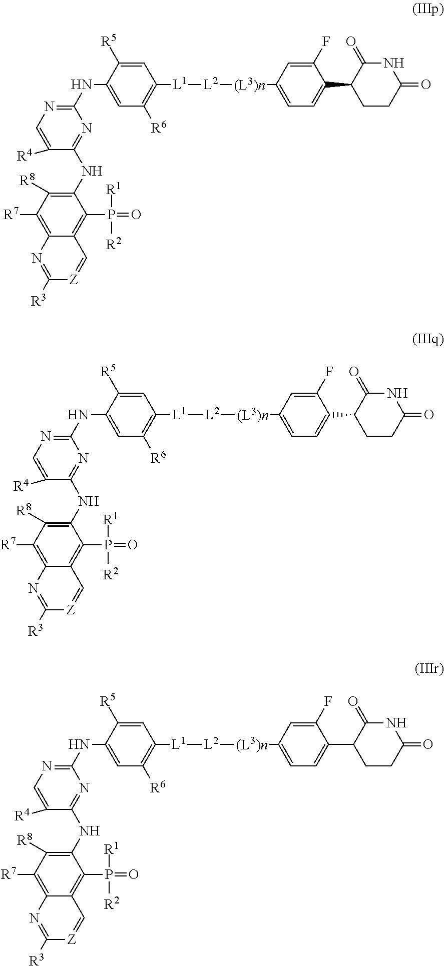 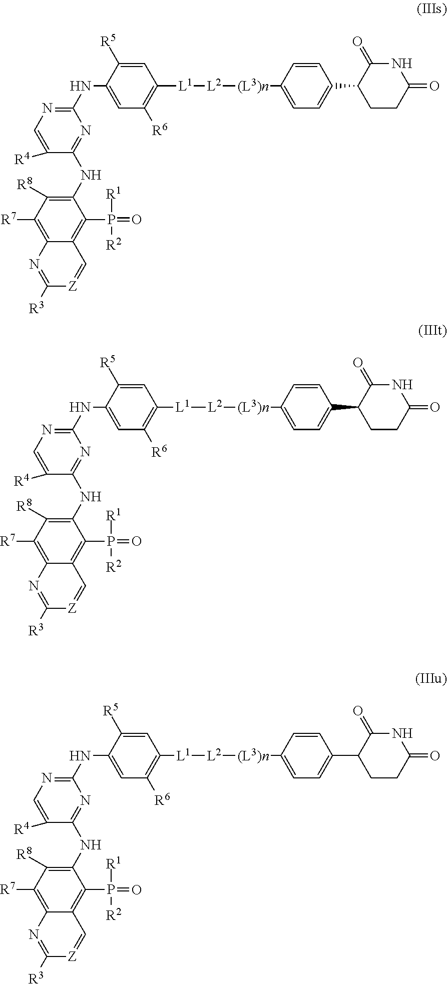
wherein
R¹, R², R³, R, R⁶, R⁷, R⁸, Z, L¹, L², L³ and n are each defined as claim 1.

4. The compound of claim 1, wherein the compound is formula (IVa) to (IVh) 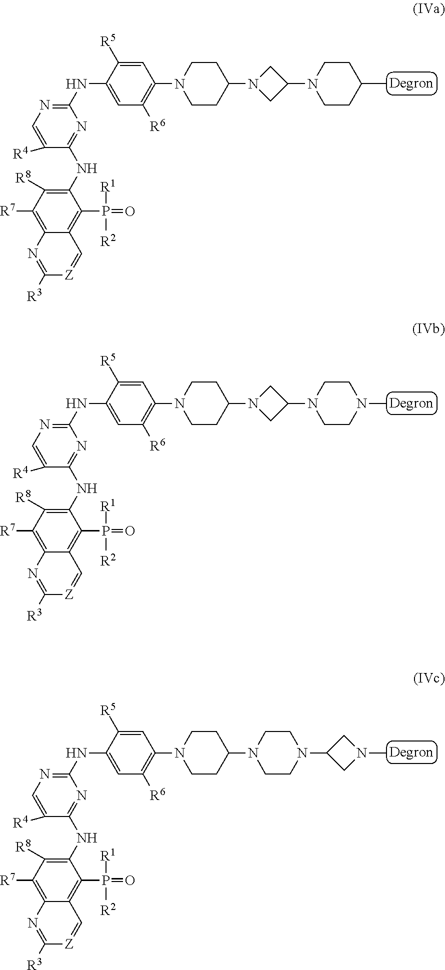 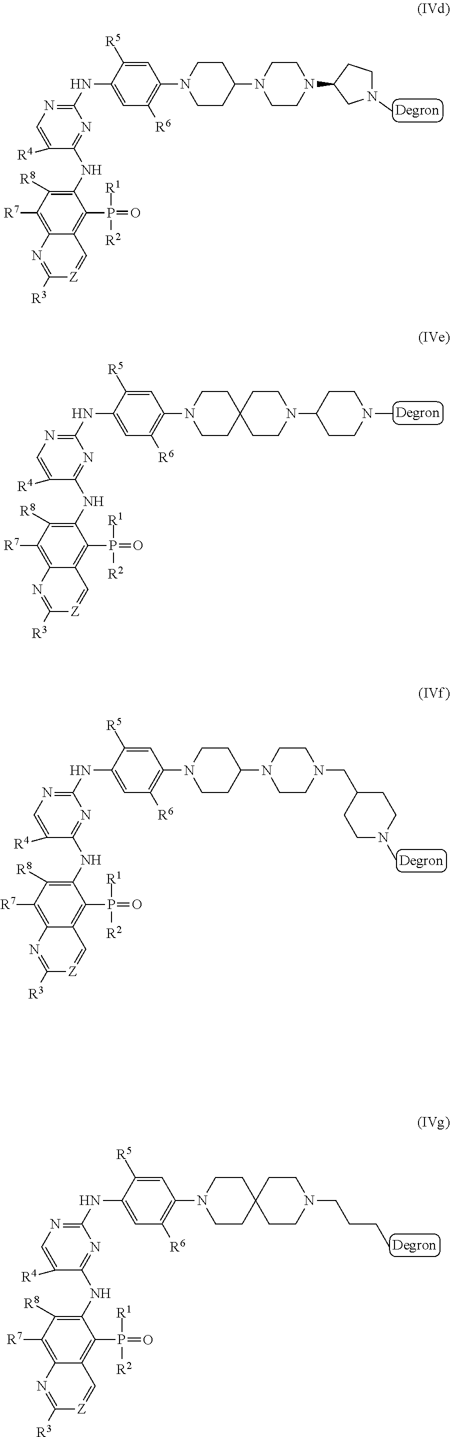 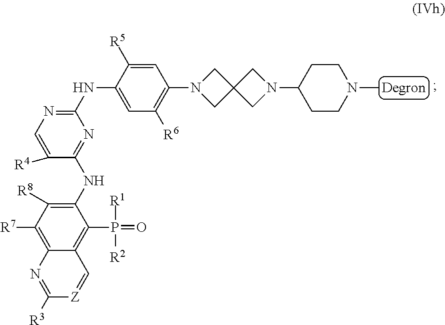
wherein
Z, R¹, R², R³, R⁴, R⁵, R⁶, R⁷, R⁸ and 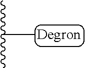
are each defined as claim 1.

5. The compound of any preceding claims, wherein Z is N or CR^{z};
R^{z} is H, methyl, ethyl, propyl, butyl, pentyl, hexyl, heptyl, octyl, cyclopropyl, cyclobutyl, cyclopentyl, cyclohexyl, cycloheptyl, cyclooctyl, —F, —Cl, —Br or —I.

6. The compound of any preceding claims, wherein Z is N or CR^{z};
R^{z} is H, methyl, ethyl, n-propyl, iso-propyl, n-butyl, iso-butyl, sec-butyl, tert-butyl, pentyl, cyclopropyl, cyclobutyl, cyclopentyl, cyclohexyl, —F, —Cl, —Br or —I.

7. The compound of any preceding claims, wherein R¹ and R² are each independently methyl, ethyl, propyl, butyl, pentyl, hexyl, heptyl, octyl, cyclopropyl, cyclobutyl, cyclopentyl, cyclohexyl, cycloheptyl or cyclooctyl.

8. The compound of any preceding claims, wherein R¹ and R² are each independently methyl.

9. The compound of any preceding claims, wherein R³ is methyl, ethyl, propyl, butyl, pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, —F, —Cl, —Br, —I, 3- to 8-membered heterocyclyl, phenyl, 5- to 12-membered heteroaryl, —OR^{3a}, —SR^{3a}, —CN, —C(O)R^{3a}, —CO₂R^{3a}, —C(O)NR^{3a}R^{3b}, —NR^{3a}R^{3b}, —NR^{3a}COR^{3b}; each of said methyl, ethyl, propyl, butyl, pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, —F, —Cl, —Br, —I, 3- to 8-membered heterocyclyl, phenyl or 5- to 12-membered heteroaryl is optionally substituted with 0, 1 or 2 R^{3c};
R^{3a} and R^{3b} are each independently selected from hydrogen, methyl, ethyl, propyl, butyl, pentyl, hexyl, heptyl, octyl, —C₁-C₈haloalkyl, —C₂-C₈alkenyl, —C₂-C₈alkynyl, C₁-C₈alkoxy-C₁-C₈alkyl-, cyclopropyl, cyclobutyl, cyclopentyl, cyclohexyl, cycloheptyl, cyclooctyl, 3- to 8-membered heterocyclyl, phenyl or 5- to 12-membered heteroaryl;
R^{3c}, at each occurrence, is independently —F, —Cl, —Br, —I, —OH, —CN, oxo (═O), methyl, ethyl, propyl, butyl, pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl.

10. The compound of any preceding claims, wherein R³ is methyl, ethyl, propyl, butyl, pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, —F, —Cl, —Br, —I, 3- to 8-membered heterocyclyl, phenyl, 5- to 12-membered heteroaryl;
preferably, R³ is methyl, ethyl, propyl (n-propyl, iso-propyl), butyl (n-butyl, iso-butyl, sec-butyl, tert-butyl), pentyl, hexyl, heptyl, octyl.

11. The compound of any preceding claims, wherein R⁴ is —F, —Cl, —Br, —I, Me, OMe.

12. The compound of any preceding claims, wherein R⁴ is —Cl or —Br.

13. The compound of any preceding claims, wherein R⁵ is hydrogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, phenyl, 5- to 12-membered heteroaryl, —NR^{5a}R^{5b}, —OR^{5a}, —SR^{5a}, or CN; each of said methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one R^{5c};
R^{5a} and R^{5b} are each independently selected from hydrogen, methyl, ethyl, propyl, butyl, pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl, each of said methyl, ethyl, propyl, butyl, pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl is optionally substituted with at least one substituent R^{5d};
R^{5c} and R^{5d} are each independently selected from —F, —Cl, —Br, —I, hydroxy, methyl, ethyl, propyl, butyl, pentyl, hexyl, heptyl, octyl, methoxy, ethoxy, propoxy, butoxy, pentoxy, hexoxy, heptoxy, octoxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl.

14. The compound of any preceding claims, wherein R⁵ is —OR^{5a};
R^{5a} is selected from hydrogen, methyl, ethyl, propyl, butyl, pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl, each of said methyl, ethyl, propyl, butyl, pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl is optionally substituted with at least one substituent R^{5d};
R^{5d} is independently selected from —F, —Cl, —Br, —I, hydroxy, methyl, ethyl, propyl, butyl, pentyl, hexyl, heptyl, octyl, methoxy, ethoxy, propoxy, butoxy, pentoxy, hexoxy, heptoxy, octoxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl;
preferably, R⁵ is methoxy, ethoxy, propoxy (n-propoxy, iso-propoxy), butoxy (n-butoxy, iso-butoxy, sec-butoxy, tert-butoxy), pentoxy, hexoxy, heptoxy or octoxy.

15. The compound of any preceding claims, wherein R⁶ is hydrogen, —F, —Cl, —Br, —I, methyl, ethyl, propyl, butyl, pentyl, hexyl, heptyl, octyl, —NR^{6a}R^{6b}, —OR^{6a}, —SR^{6a}, cyclopropyl, cyclobutyl, cyclopentyl, cyclohexyl, cycloheptyl, cyclooctyl, 3- to 8-membered heterocyclyl, phenyl, 5- to 12-membered heteroaryl, or CN; each of said methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one R^{6c};
R^{6a} and R^{6b} are each independently selected from hydrogen, methyl, ethyl, propyl, butyl, pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl, each of said methyl, ethyl, propyl, butyl, pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent R^{6d};
R^{6c} and R^{6d} are each independently selected from —F, —Cl, —Br, —I, methyl, ethyl, propyl, butyl, pentyl, hexyl, heptyl, octyl, methoxy, ethoxy, propoxy, butoxy, pentoxy, hexoxy, heptoxy, octoxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl.

16. The compound of any preceding claims, wherein R⁶ is hydrogen, methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, phenyl, 5- to 12-membered heteroaryl, or CN; each of said methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one R^{6c};
R^{6c} is independently selected from —F, —Cl, —Br, —I, methyl, ethyl, propyl, butyl, pentyl, hexyl, heptyl, octyl, methoxy, ethoxy, propoxy, butoxy, pentoxy, hexoxy, heptoxy, octoxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl;
preferably, R⁶ is hydrogen, methyl, ethyl, propyl (n-propyl, iso-propyl), butyl (n-butyl, sec-butyl, iso-butyl, tert-butyl), pentyl, hexyl, heptyl, octyl, cyclopropyl, cyclobutyl, cyclopentyl, cyclohexyl, cycloheptyl, cyclooctyl.

17. The compound of any preceding claims, wherein R⁷ and R⁸ are each independently selected from hydrogen, —F, —Cl, —Br, —I, methyl, ethyl, propyl, butyl, pentyl, hexyl, heptyl, octyl, —NR^{7a}R^{7b}, —OR^{7a}, cyclopropyl, cyclobutyl, cyclopentyl, cyclohexyl, cycloheptyl, cyclooctyl, 3- to 8-membered heterocyclyl, phenyl, 5- to 12-membered heteroaryl or —CN; each of said methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent R^{7c}; or
R^{7a} and R^{7b} are each independently selected from hydrogen, methyl, ethyl, propyl, butyl, pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl, each of said methyl, ethyl, propyl, butyl, pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent R^{7d}; or
R^{7c} and R^{7d} are each independently —F, —Cl, —Br, —I, hydroxy, methyl, ethyl, propyl, butyl, pentyl, hexyl, heptyl, octyl, methoxy, ethoxy, propoxy, butoxy, pentoxy, hexoxy, heptoxy, octoxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl.

18. The compound of any preceding claims, wherein R¹ and R⁸ are each independently selected from hydrogen, —F, —Cl, —Br, —I, methyl, ethyl, propyl, butyl, pentyl, hexyl, heptyl, octyl, methoxy, ethoxy, propoxy, butoxy, pentoxy, hexoxy, heptoxy, octoxy, cyclopropyl, cyclobutyl, cyclopentyl, cyclohexyl, cycloheptyl, cyclooctyl, 3- to 8-membered heterocyclyl, phenyl or 5- to 12-membered heteroaryl;
preferably, R¹ and R⁸ are each hydrogen, —F, —Cl, —Br or —I.

19. The compound of any preceding claims, wherein the 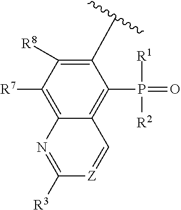
moiety is selected from:

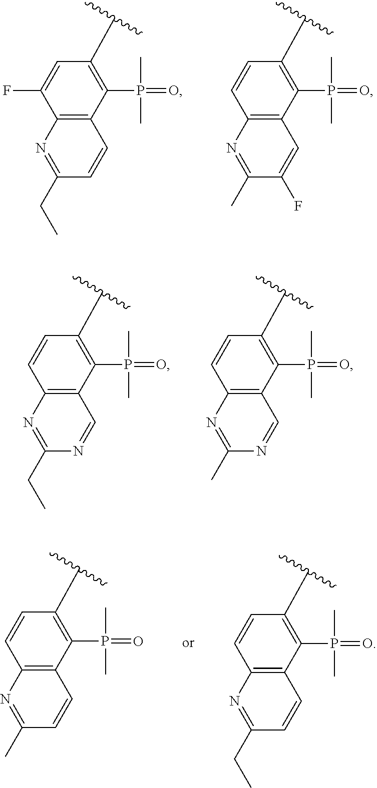 [SMILES: *c1cc(F)c2nc(CC)ccc2c1P(C)(C)=O ; *c1ccc2nc(C)c(F)cc2c1P(C)(C)=O ; *c1ccc2nc(CC)ncc2c1P(C)(C)=O ; *c1ccc2nc(C)ncc2c1P(C)(C)=O ; *c1ccc2nc(C)ccc2c1P(C)(C)=O ; *c1ccc2nc(CC)ccc2c1P(C)(C)=O]

20. The compound of any preceding claims, wherein the 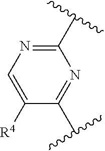
moiety is selected from:

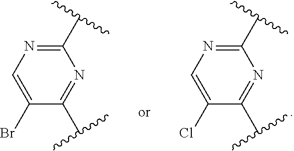 [SMILES: *c1ncc(Br)c(*)n1 ; *c1ncc(Cl)c(*)n1]

21. The compound of any preceding claims, wherein the 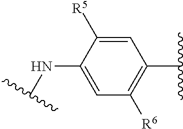
moiety is selected from:

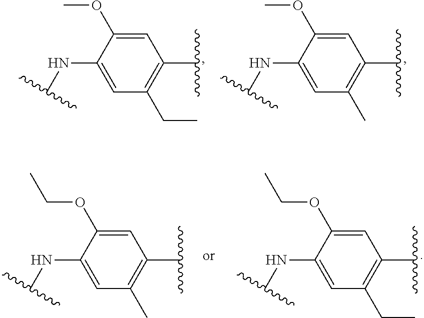 [SMILES: *Nc1cc(CC)c(*)cc1OC ; *Nc1cc(C)c(*)cc1OC ; *Nc1cc(C)c(*)cc1OCC ; *Nc1cc(CC)c(*)cc1OCC]

22. The compound of any preceding claims, wherein the 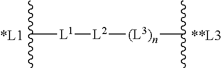
moiety is selected from:

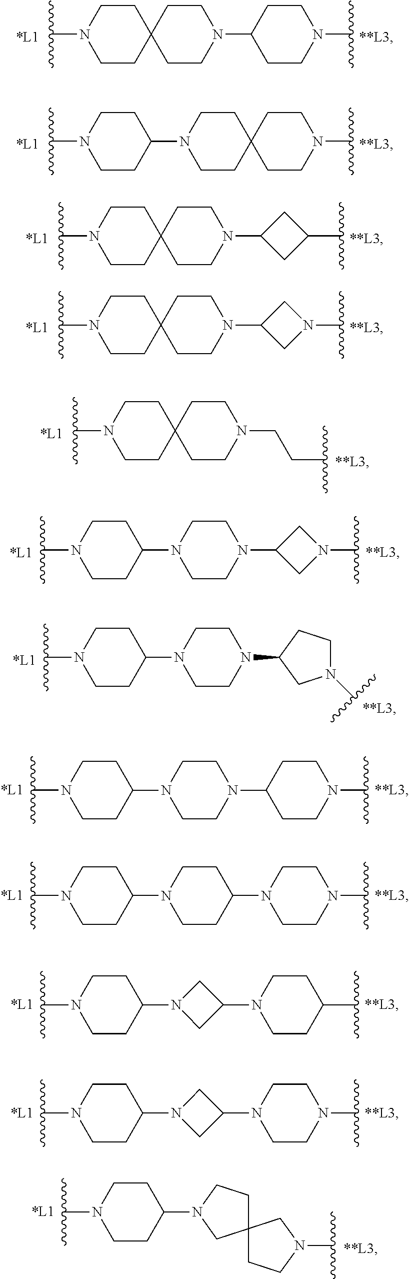

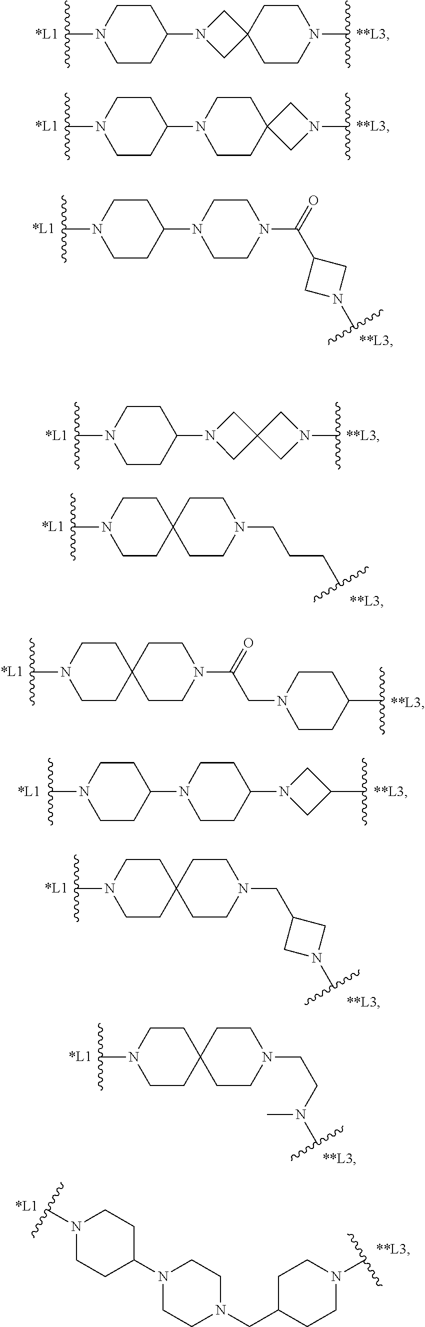

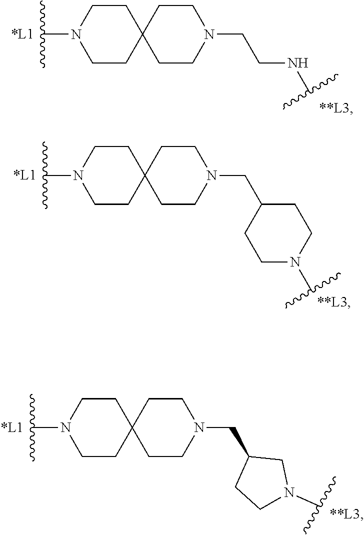 [SMILES: *NCCN1CCC2(CCN(*)CC2)CC1 ; *N1CCC(CN2CCC3(CCN(*)CC3)CC2)CC1 ; *N1CCC2(CC1)CCN(C[C@@H]1CCN(*)C1)CC2]

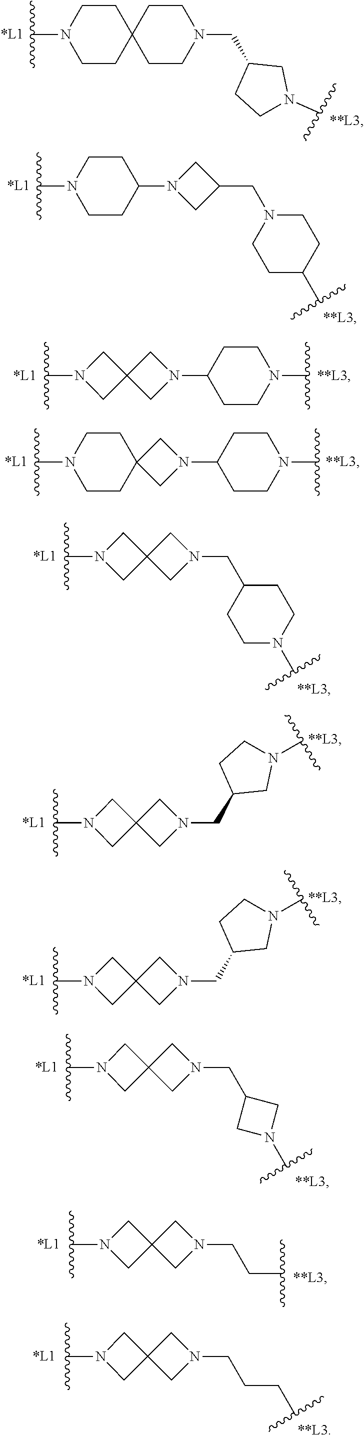

23. The compound of any preceding claims, wherein the compound is selected from

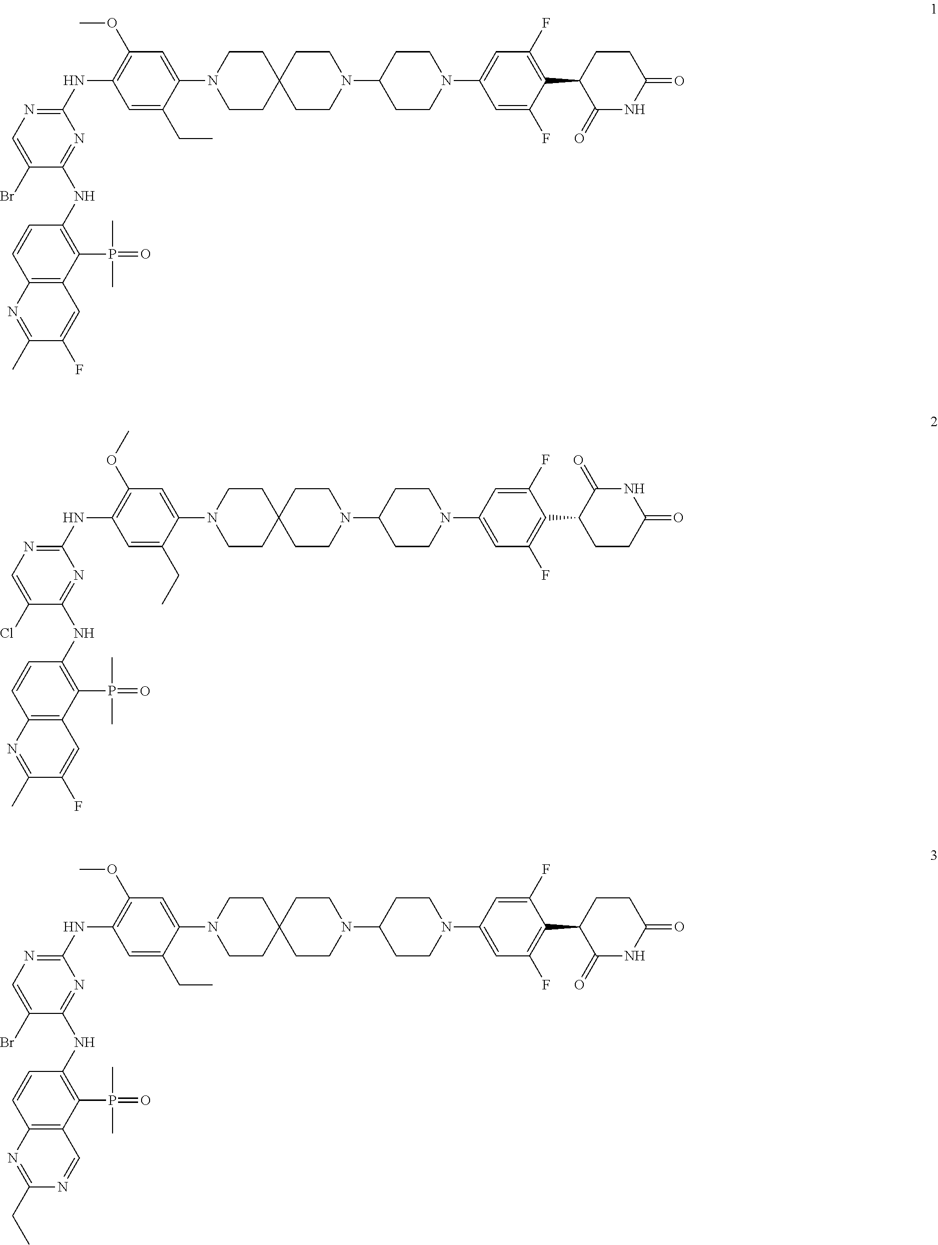

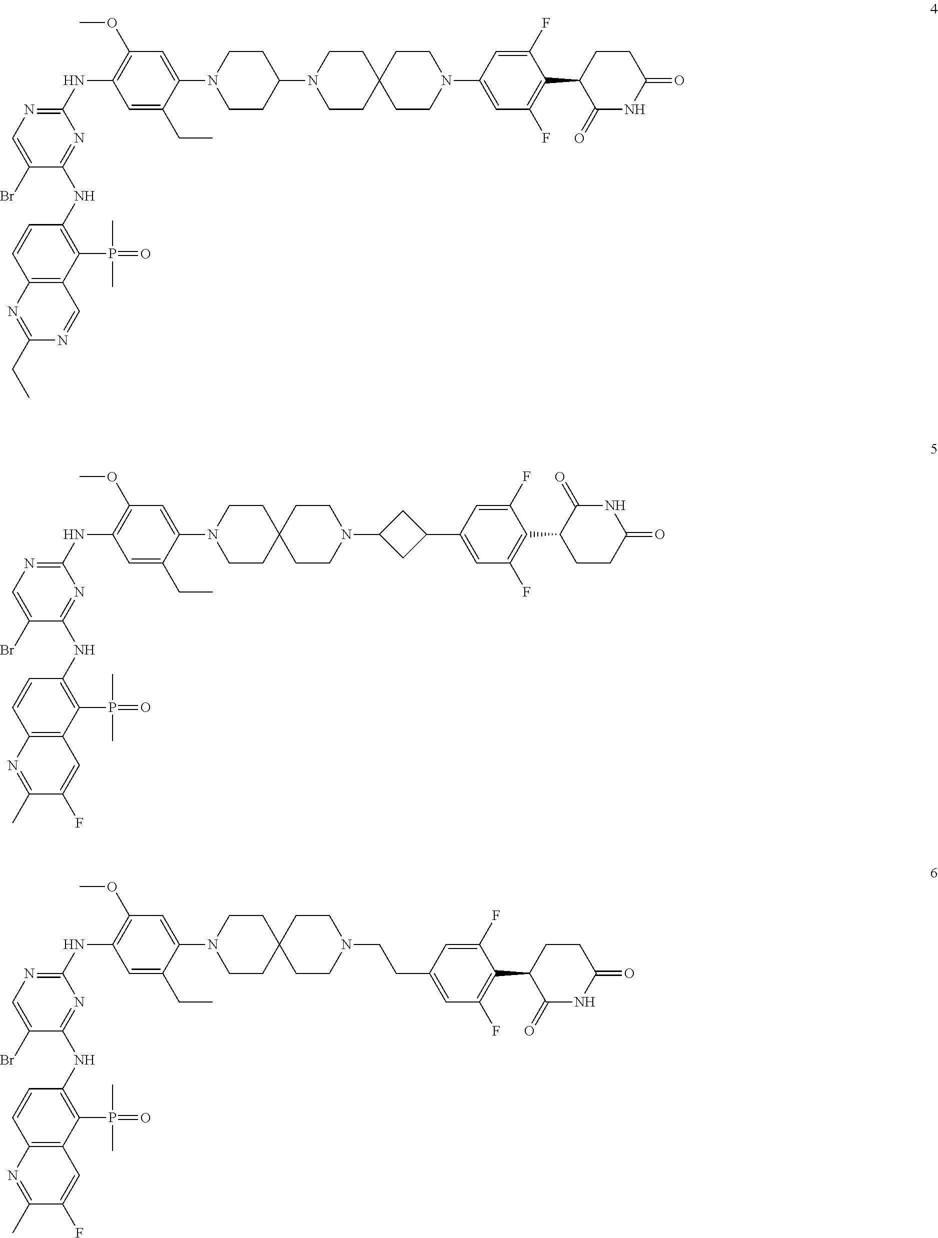

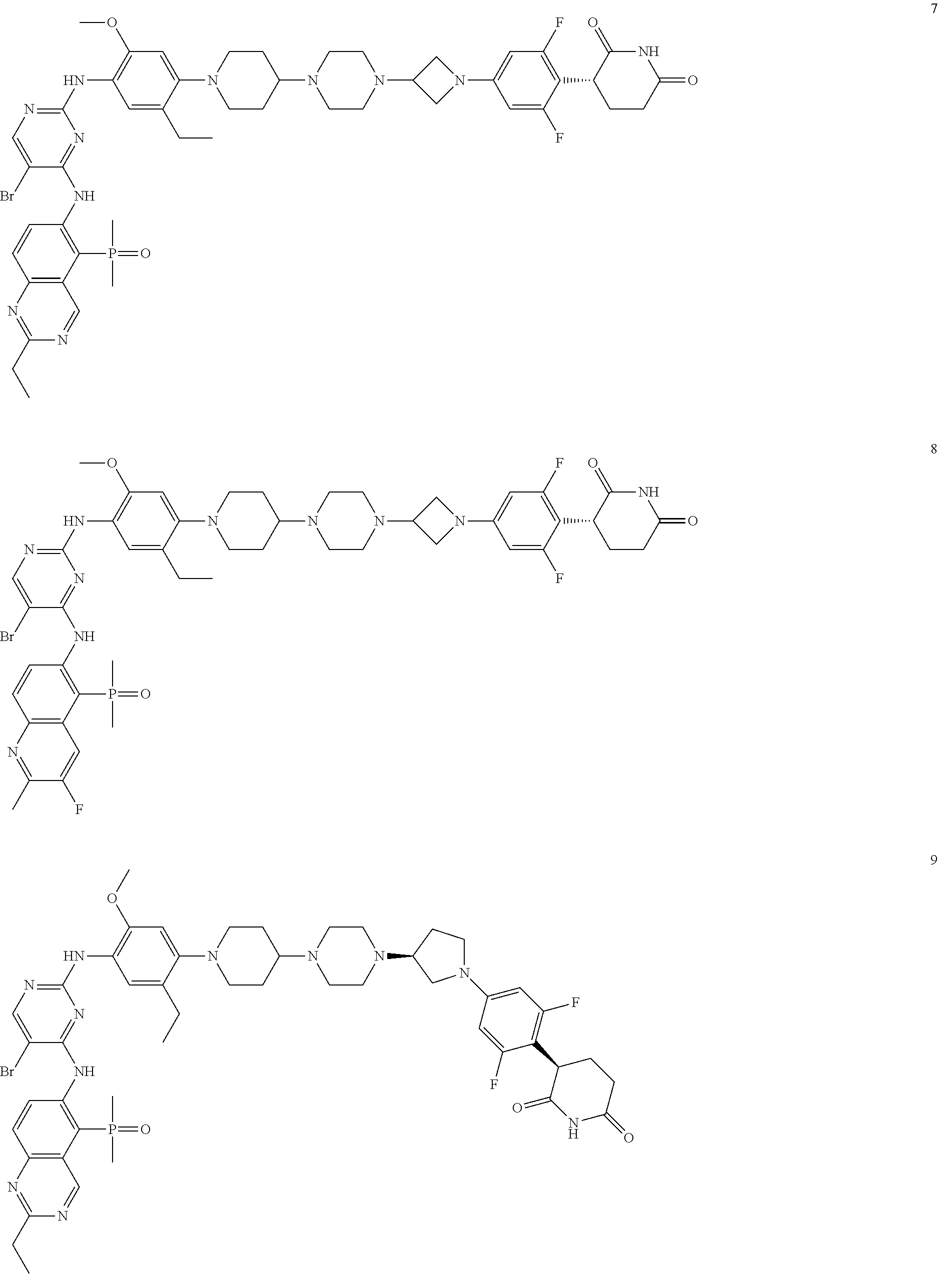 [SMILES: CCc1ncc2c(P(C)(C)=O)c(Nc3nc(Nc4cc(CC)c(N5CCC(N6CCN(C7CN(c8cc(F)c([C@H]9CCC(=O)NC9=O)c(F)c8)C7)CC6)CC5)cc4OC)ncc3Br)ccc2n1 ; CCc1cc(Nc2ncc(Br)c(Nc3ccc4nc(C)c(F)cc4c3P(C)(C)=O)n2)c(OC)cc1N1CCC(N2CCN(C3CN(c4cc(F)c([C@H]5CCC(=O)NC5=O)c(F)c4)C3)CC2)CC1 ; CCc1ncc2c(P(C)(C)=O)c(Nc3nc(Nc4cc(CC)c(N5CCC(N6CCN([C@H]7CCN(c8cc(F)c([C@H]9CCC(=O)NC9=O)c(F)c8)C7)CC6)CC5)cc4OC)ncc3Br)ccc2n1]

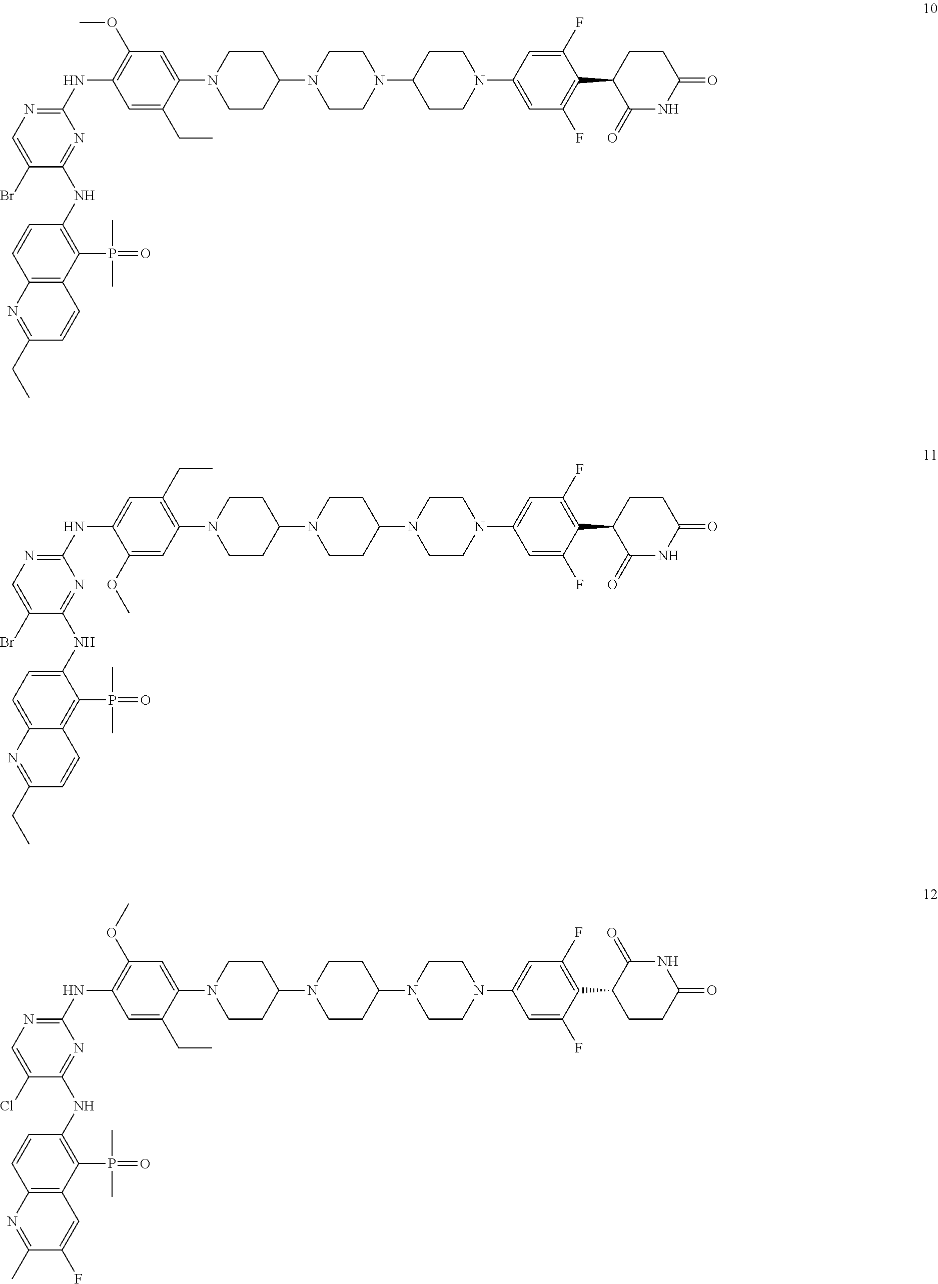

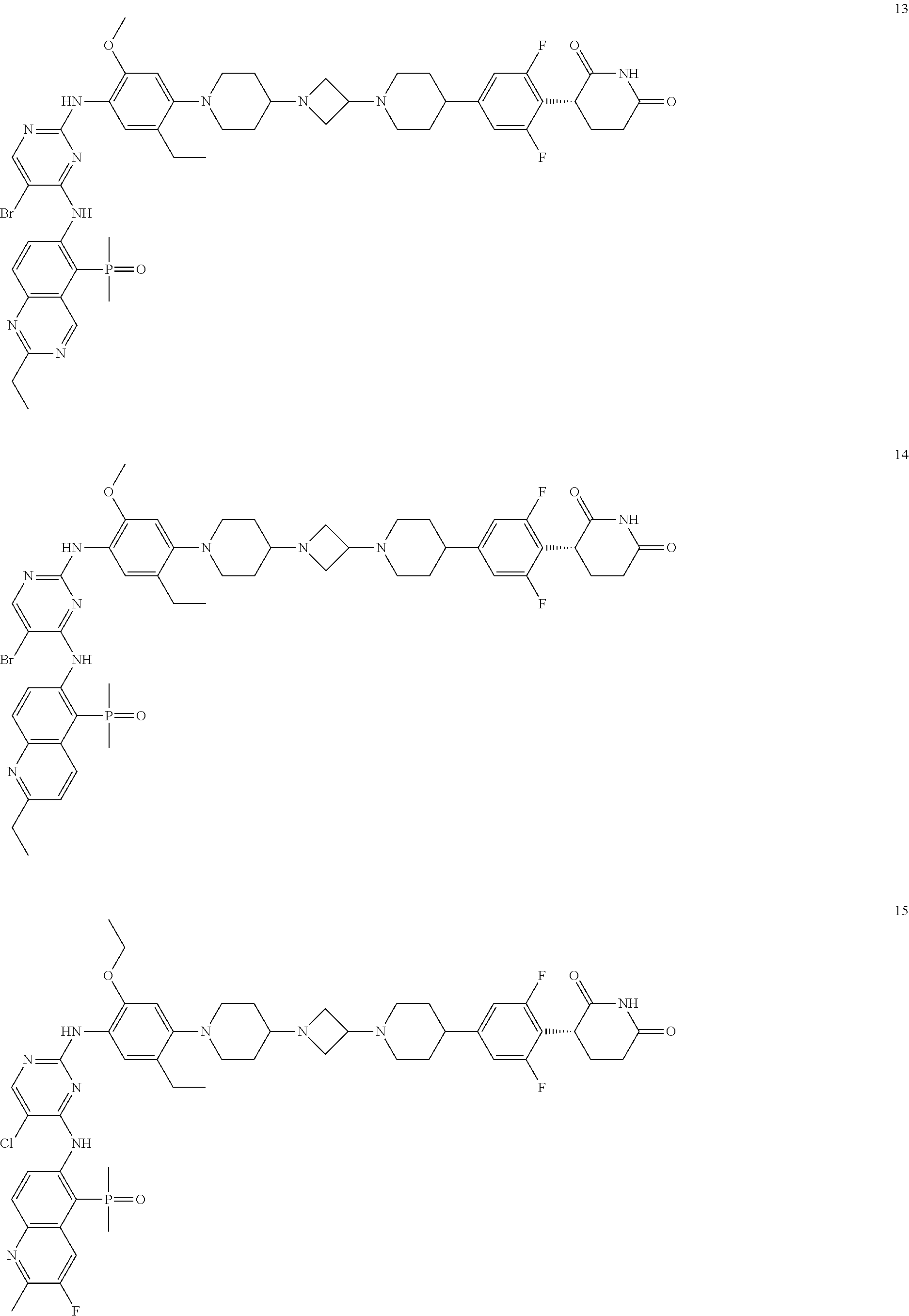

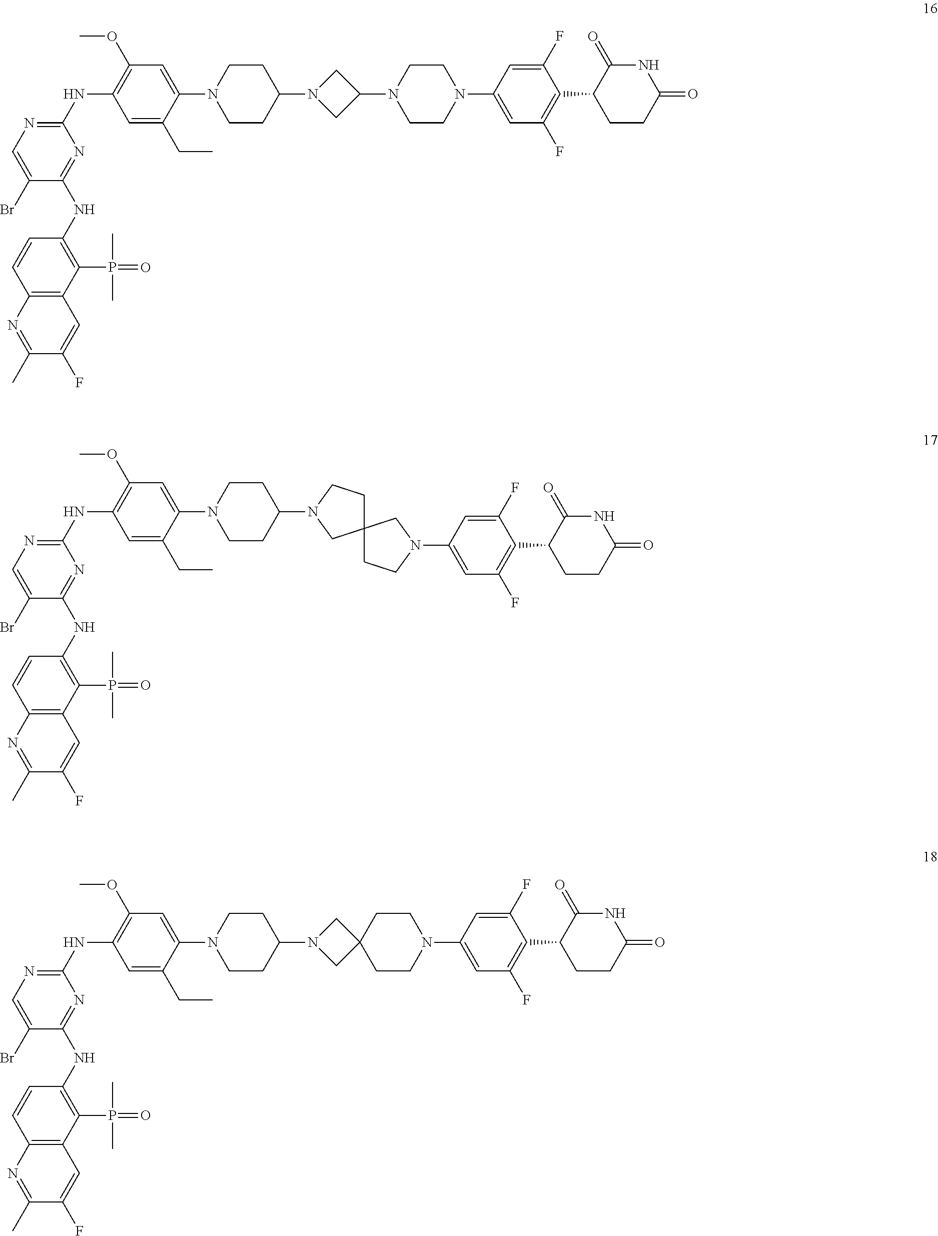

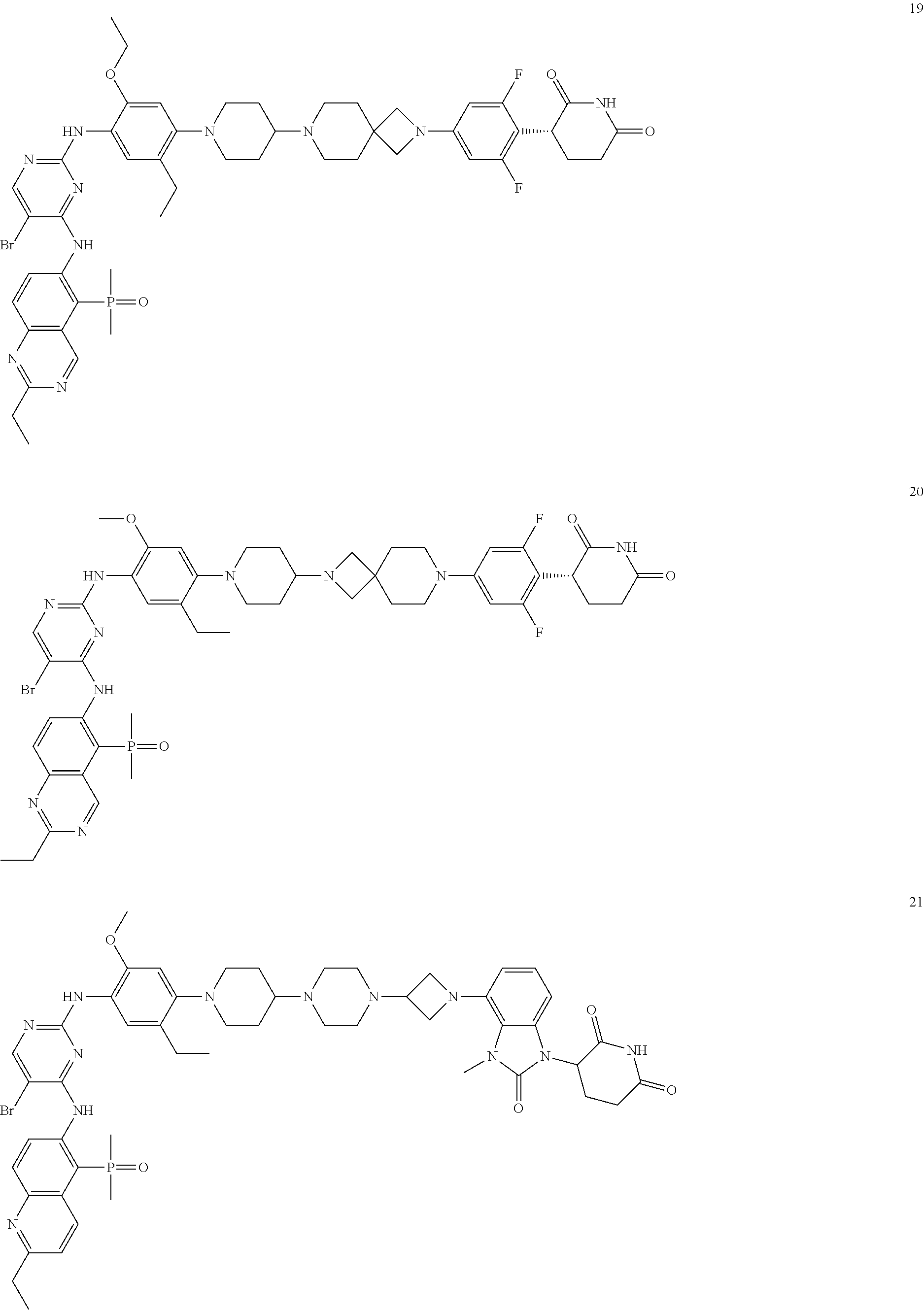

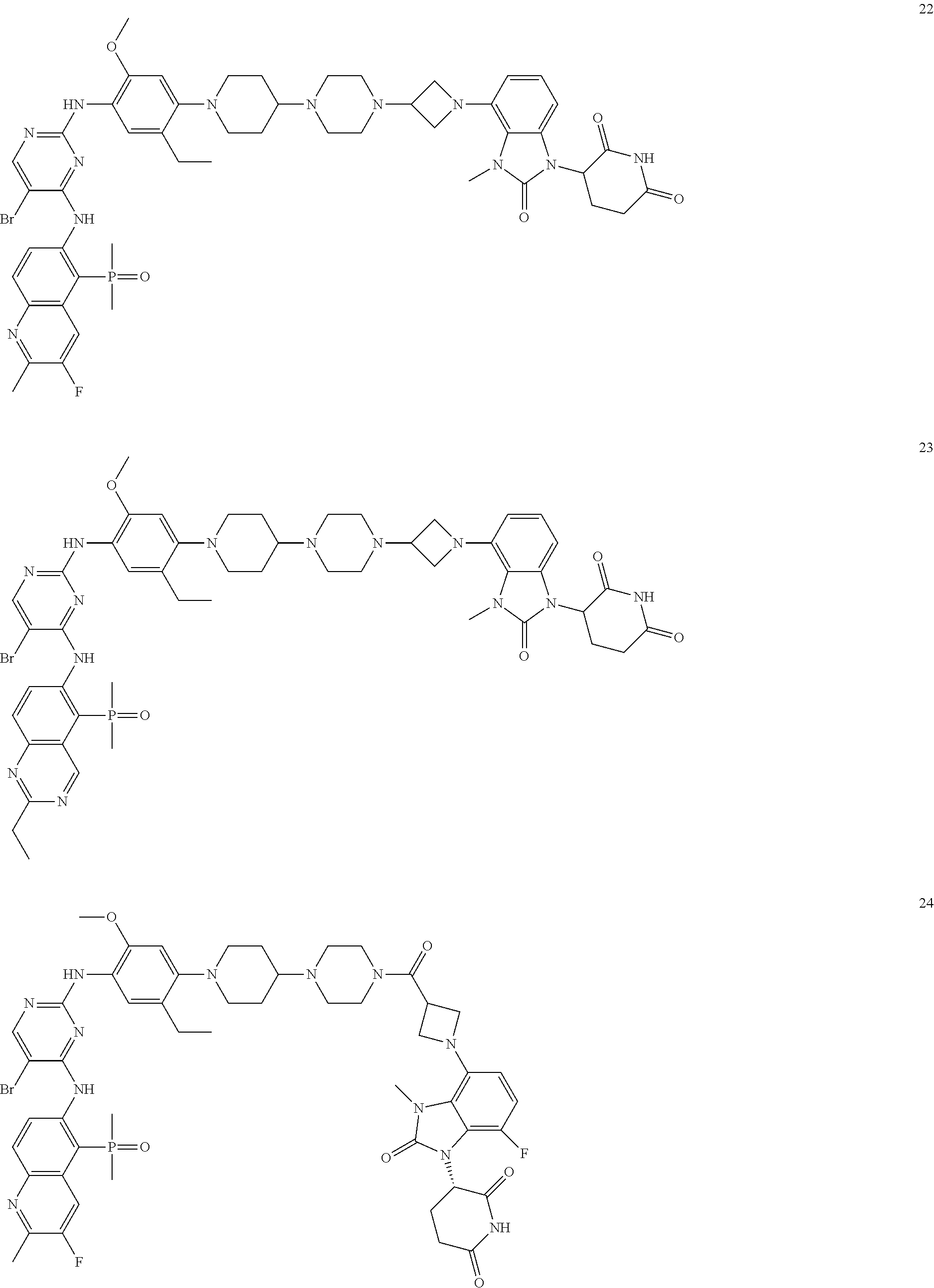

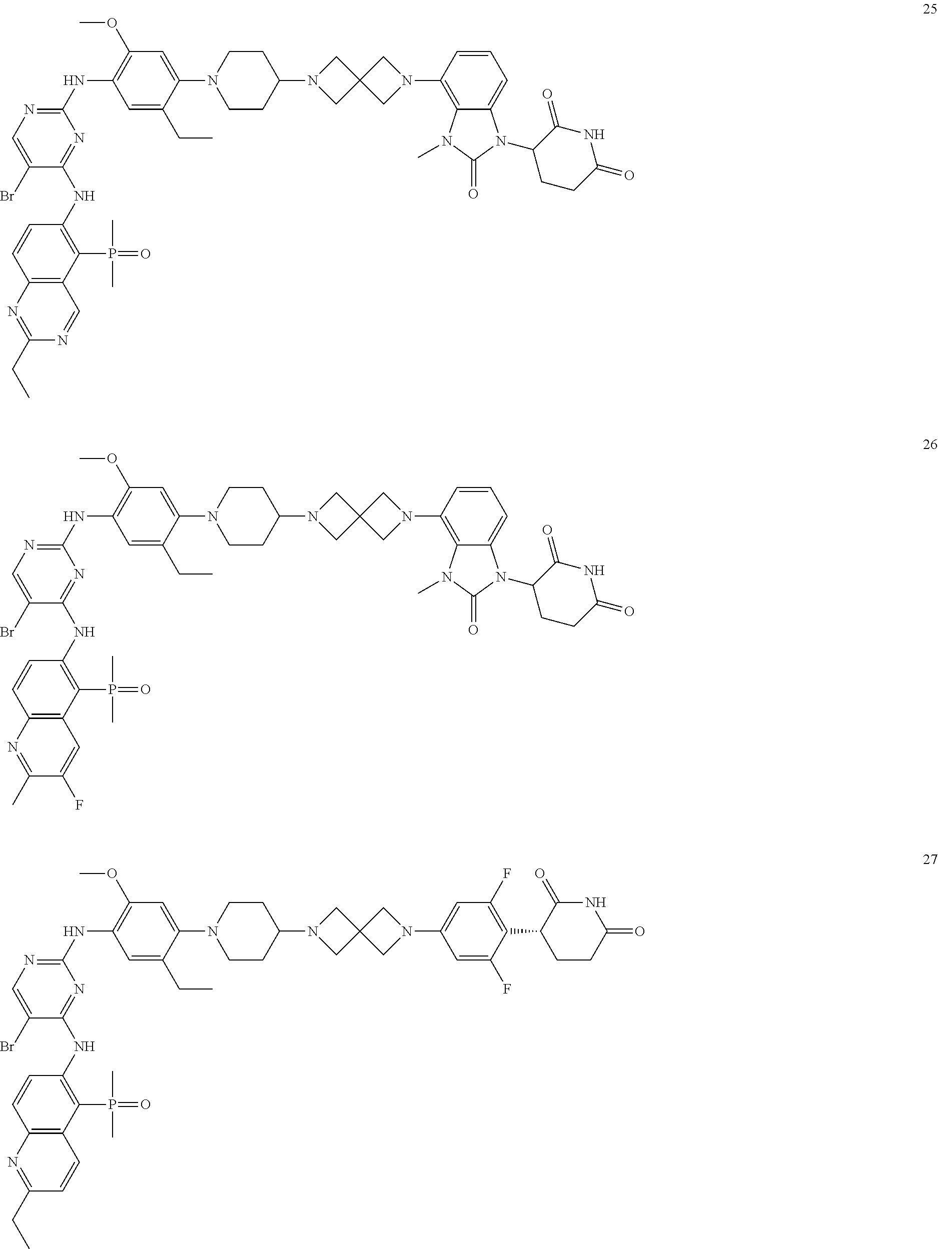

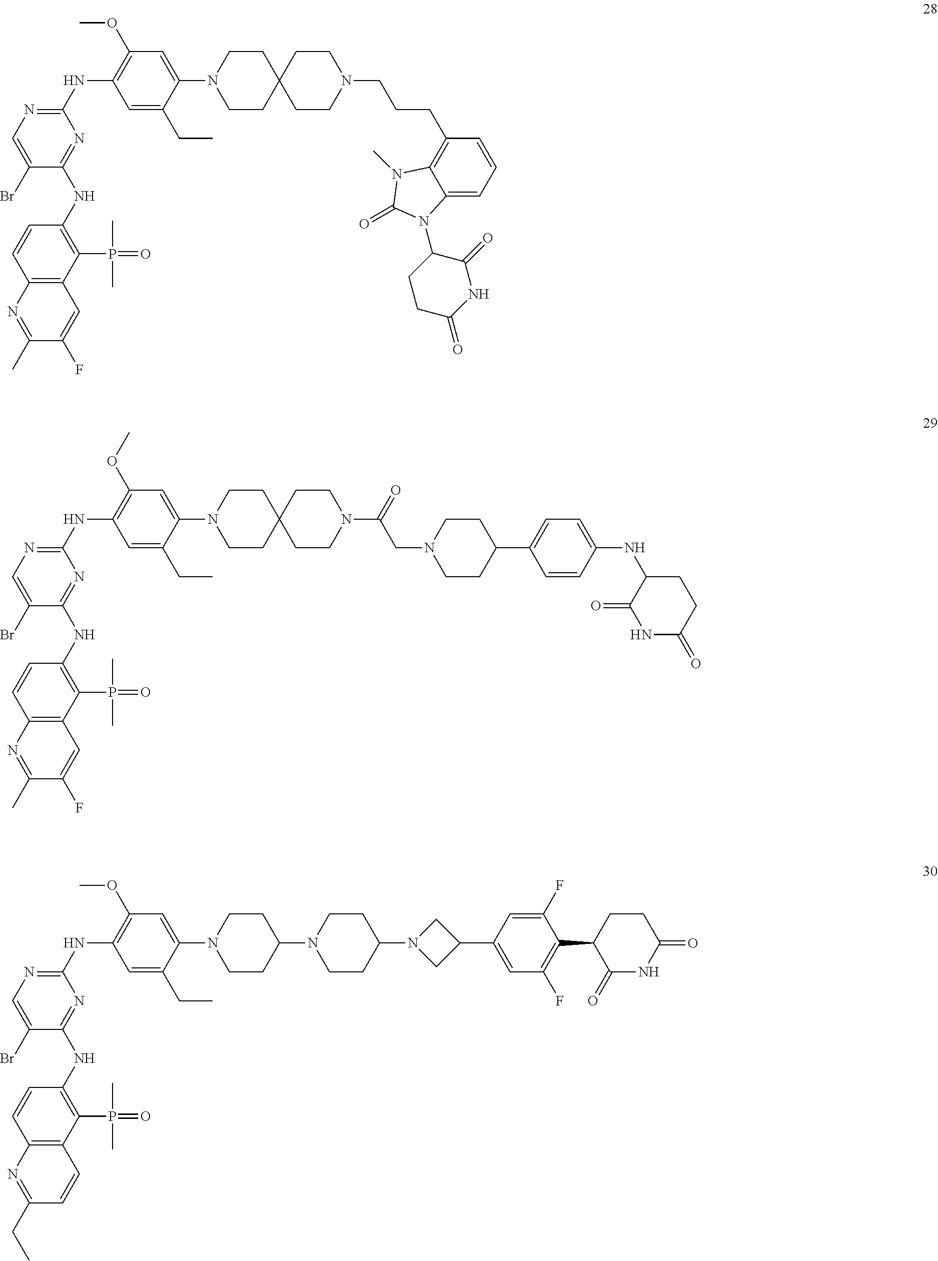

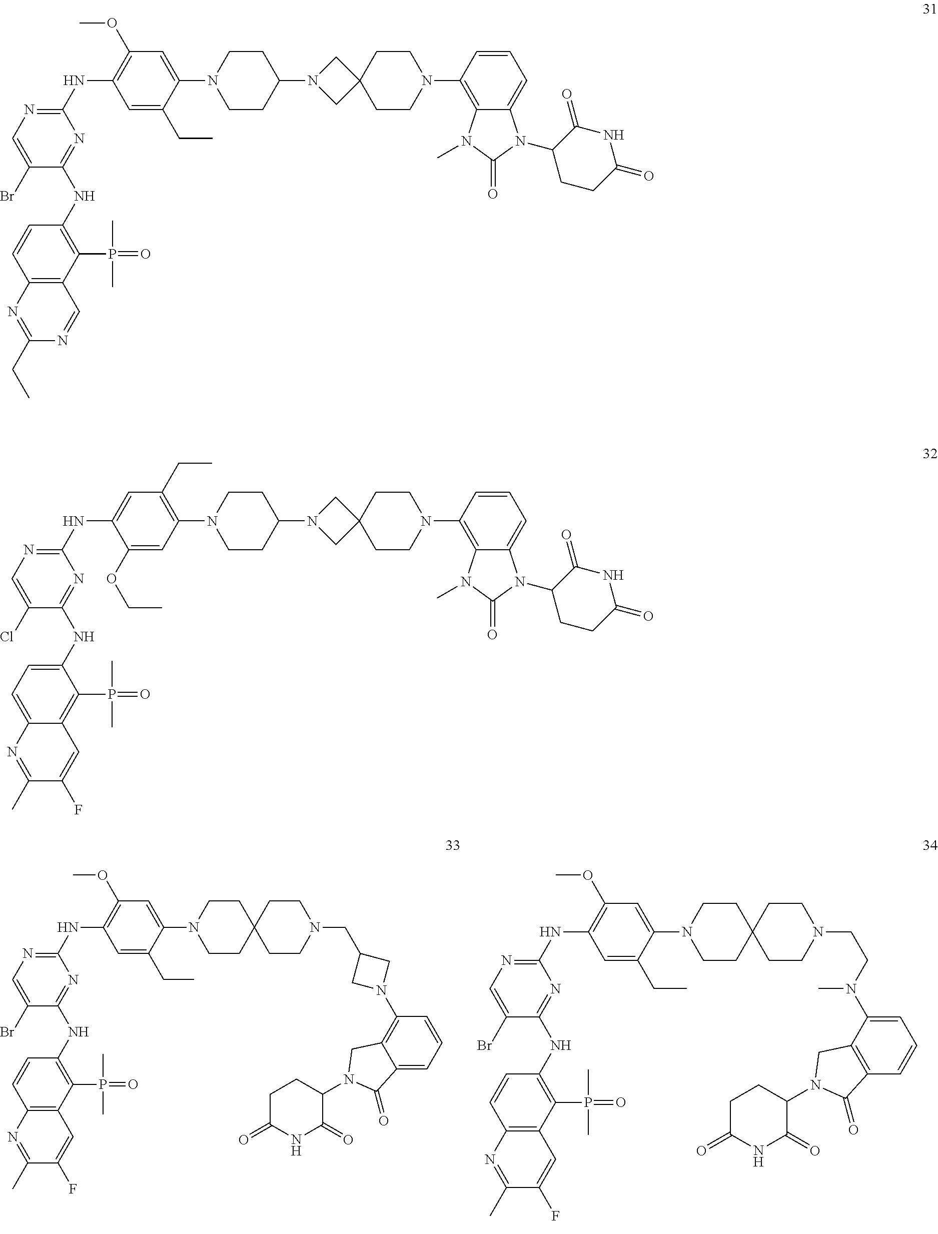

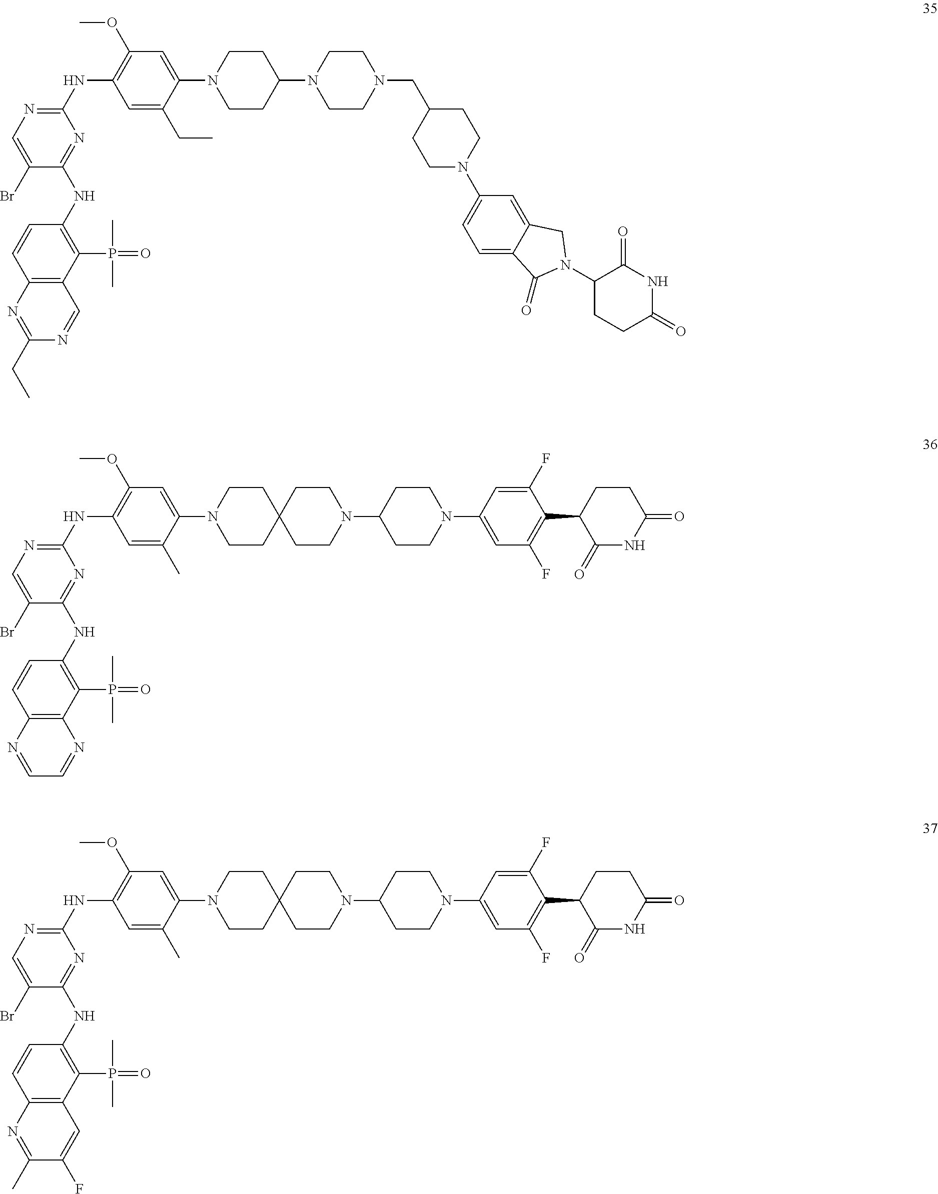 [SMILES: CCc1ncc2c(P(C)(C)=O)c(Nc3nc(Nc4cc(CC)c(N5CCC(N6CCN(CC7CCN(c8ccc9c(c8)CN(C8CCC(=O)NC8=O)C9=O)CC7)CC6)CC5)cc4OC)ncc3Br)ccc2n1 ; COc1cc(N2CCC3(CC2)CCN(C2CCN(c4cc(F)c([C@H]5CCC(=O)NC5=O)c(F)c4)CC2)CC3)c(C)cc1Nc1ncc(Br)c(Nc2ccc3nccnc3c2P(C)(C)=O)n1 ; COc1cc(N2CCC3(CC2)CCN(C2CCN(c4cc(F)c([C@H]5CCC(=O)NC5=O)c(F)c4)CC2)CC3)c(C)cc1Nc1ncc(Br)c(Nc2ccc3nc(C)c(F)cc3c2P(C)(C)=O)n1]

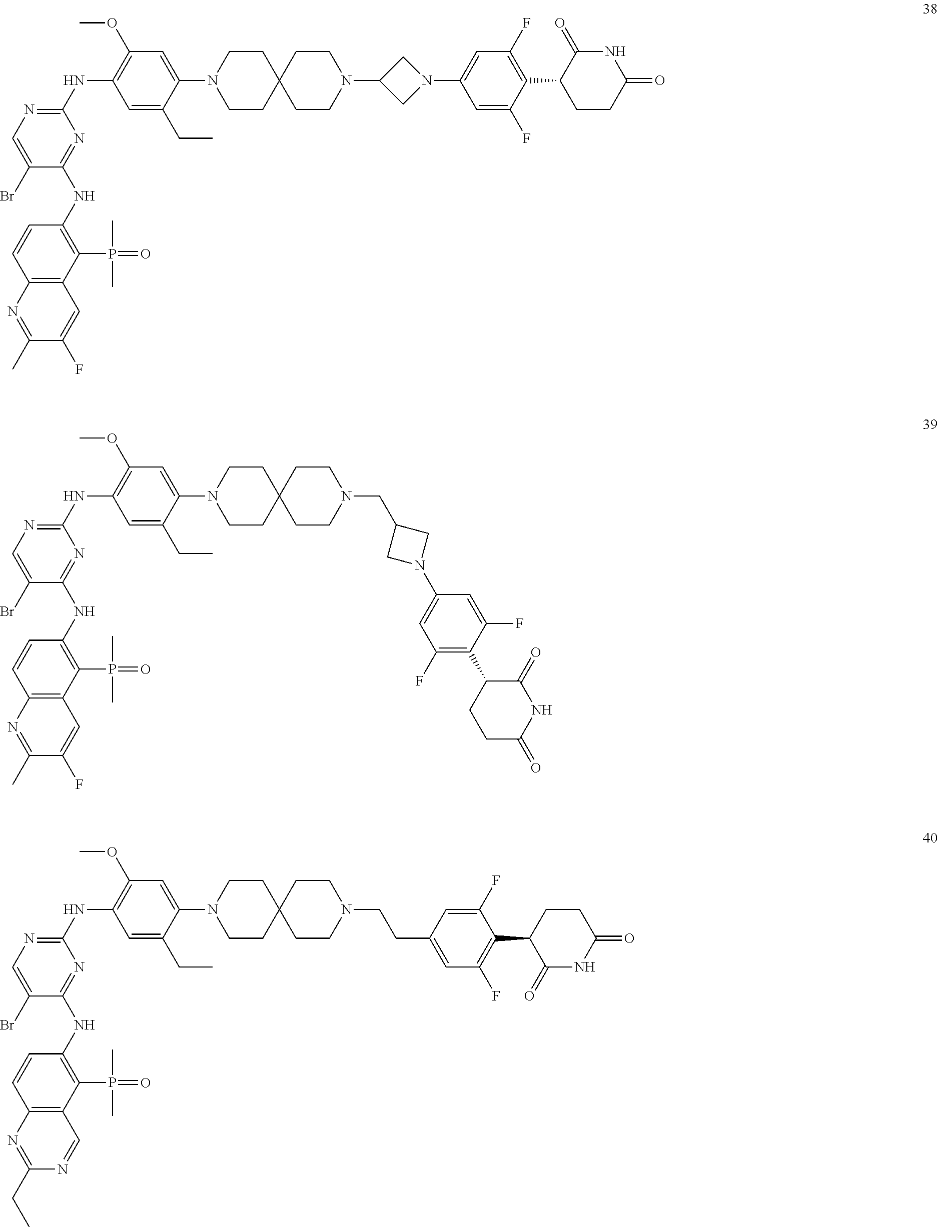 [SMILES: CCc1cc(Nc2ncc(Br)c(Nc3ccc4nc(C)c(F)cc4c3P(C)(C)=O)n2)c(OC)cc1N1CCC2(CC1)CCN(C1CN(c3cc(F)c([C@H]4CCC(=O)NC4=O)c(F)c3)C1)CC2 ; CCc1cc(Nc2ncc(Br)c(Nc3ccc4nc(C)c(F)cc4c3P(C)(C)=O)n2)c(OC)cc1N1CCC2(CCN(CC3CN(c4cc(F)c([C@H]5CCC(=O)NC5=O)c(F)c4)C3)CC2)CC1 ; CCc1ncc2c(P(C)(C)=O)c(Nc3nc(Nc4cc(CC)c(N5CCC6(CCN(CCc7cc(F)c([C@H]8CCC(=O)NC8=O)c(F)c7)CC6)CC5)cc4OC)ncc3Br)ccc2n1]

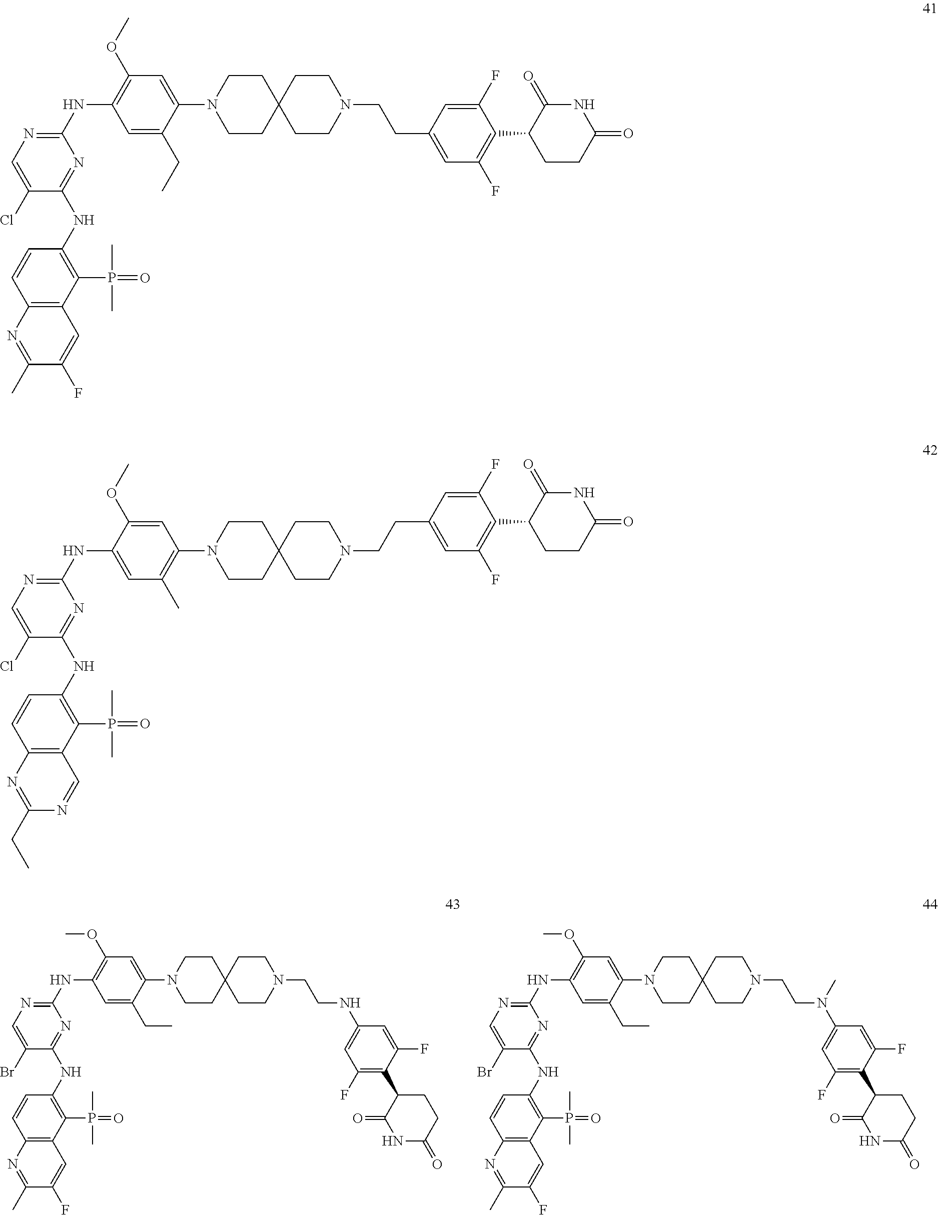 [SMILES: CCc1cc(Nc2ncc(Cl)c(Nc3ccc4nc(C)c(F)cc4c3P(C)(C)=O)n2)c(OC)cc1N1CCC2(CCN(CCc3cc(F)c([C@H]4CCC(=O)NC4=O)c(F)c3)CC2)CC1 ; CCc1ncc2c(P(C)(C)=O)c(Nc3nc(Nc4cc(C)c(N5CCC6(CCN(CCc7cc(F)c([C@H]8CCC(=O)NC8=O)c(F)c7)CC6)CC5)cc4OC)ncc3Cl)ccc2n1 ; CCc1cc(Nc2ncc(Br)c(Nc3ccc4nc(C)c(F)cc4c3P(C)(C)=O)n2)c(OC)cc1N1CCC2(CCN(CCNc3cc(F)c([C@H]4CCC(=O)NC4=O)c(F)c3)CC2)CC1 ; CCc1cc(Nc2ncc(Br)c(Nc3ccc4nc(C)c(F)cc4c3P(C)(C)=O)n2)c(OC)cc1N1CCC2(CCN(CCN(C)c3cc(F)c([C@H]4CCC(=O)NC4=O)c(F)c3)CC2)CC1]

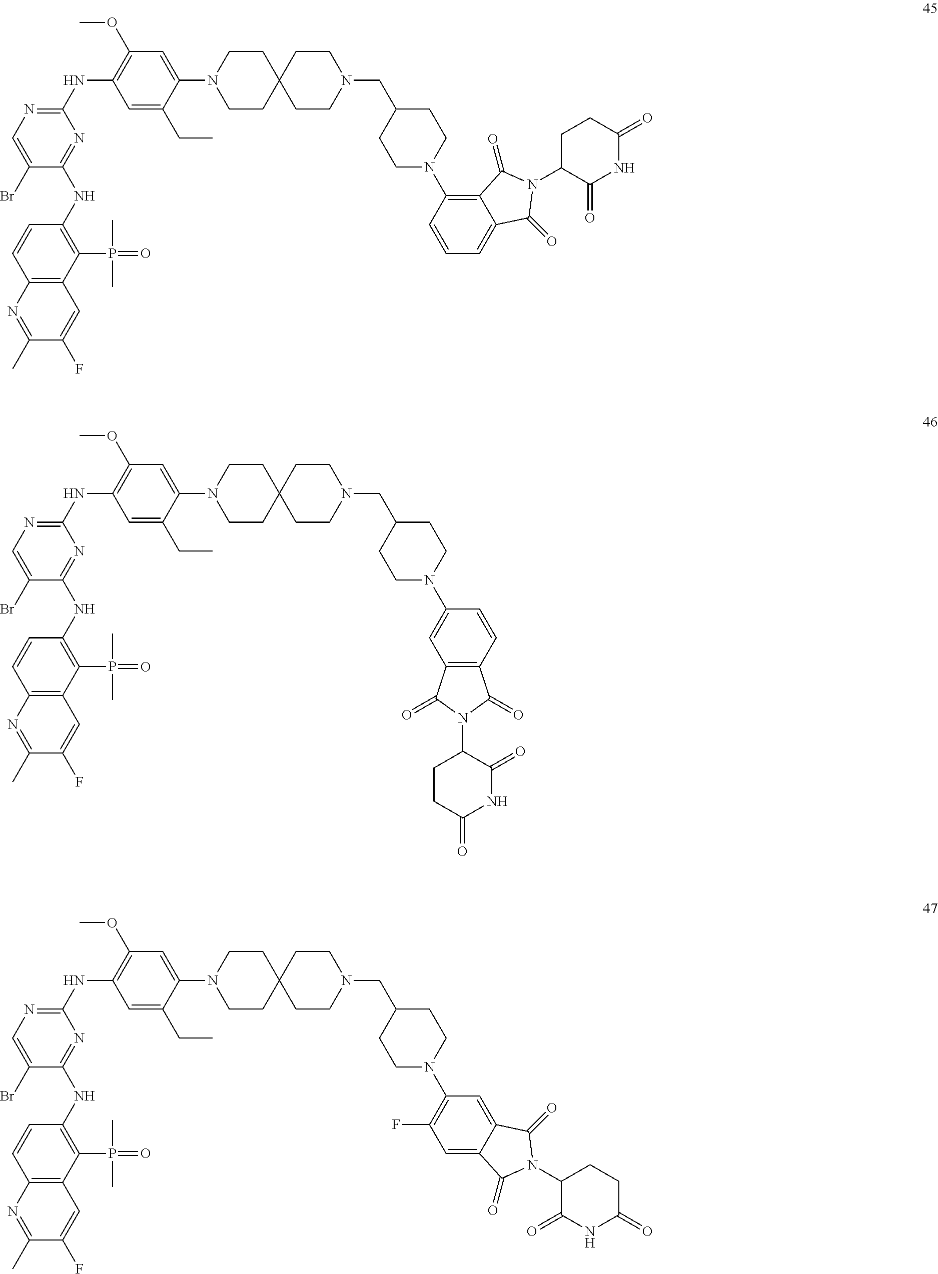 [SMILES: CCc1cc(Nc2ncc(Br)c(Nc3ccc4nc(C)c(F)cc4c3P(C)(C)=O)n2)c(OC)cc1N1CCC2(CCN(CC3CCN(c4cccc5c4C(=O)N(C4CCC(=O)NC4=O)C5=O)CC3)CC2)CC1 ; CCc1cc(Nc2ncc(Br)c(Nc3ccc4nc(C)c(F)cc4c3P(C)(C)=O)n2)c(OC)cc1N1CCC2(CCN(CC3CCN(c4ccc5c(c4)C(=O)N(C4CCC(=O)NC4=O)C5=O)CC3)CC2)CC1 ; CCc1cc(Nc2ncc(Br)c(Nc3ccc4nc(C)c(F)cc4c3P(C)(C)=O)n2)c(OC)cc1N1CCC2(CCN(CC3CCN(c4cc5c(cc4F)C(=O)N(C4CCC(=O)NC4=O)C5=O)CC3)CC2)CC1]

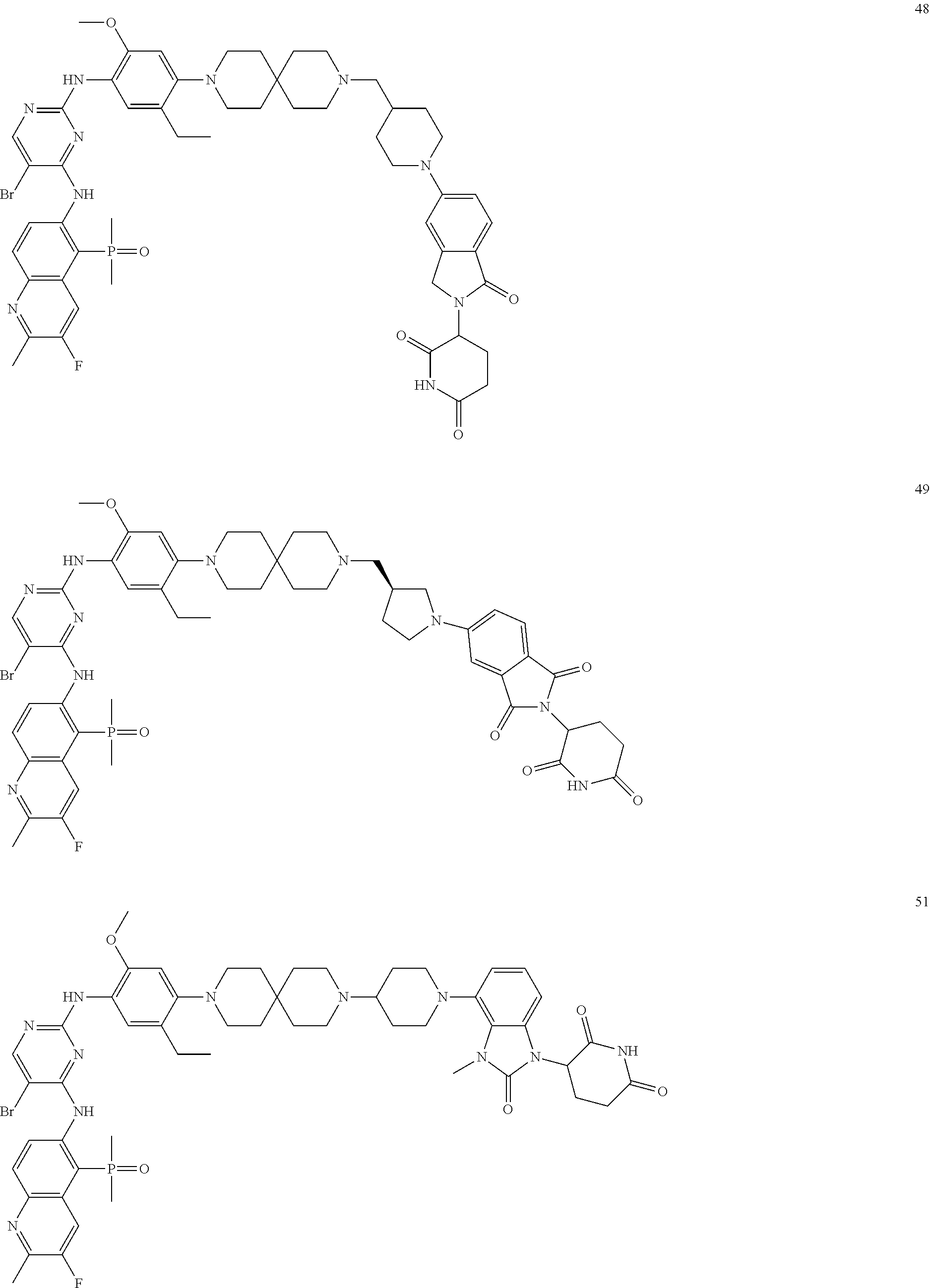 [SMILES: CCc1cc(Nc2ncc(Br)c(Nc3ccc4nc(C)c(F)cc4c3P(C)(C)=O)n2)c(OC)cc1N1CCC2(CCN(CC3CCN(c4ccc5c(c4)CN(C4CCC(=O)NC4=O)C5=O)CC3)CC2)CC1 ; CCc1cc(Nc2ncc(Br)c(Nc3ccc4nc(C)c(F)cc4c3P(C)(C)=O)n2)c(OC)cc1N1CCC2(CCN(C[C@@H]3CCN(c4ccc5c(c4)C(=O)N(C4CCC(=O)NC4=O)C5=O)C3)CC2)CC1 ; CCc1cc(Nc2ncc(Br)c(Nc3ccc4nc(C)c(F)cc4c3P(C)(C)=O)n2)c(OC)cc1N1CCC2(CC1)CCN(C1CCN(c3cccc4c3n(C)c(=O)n4C3CCC(=O)NC3=O)CC1)CC2]

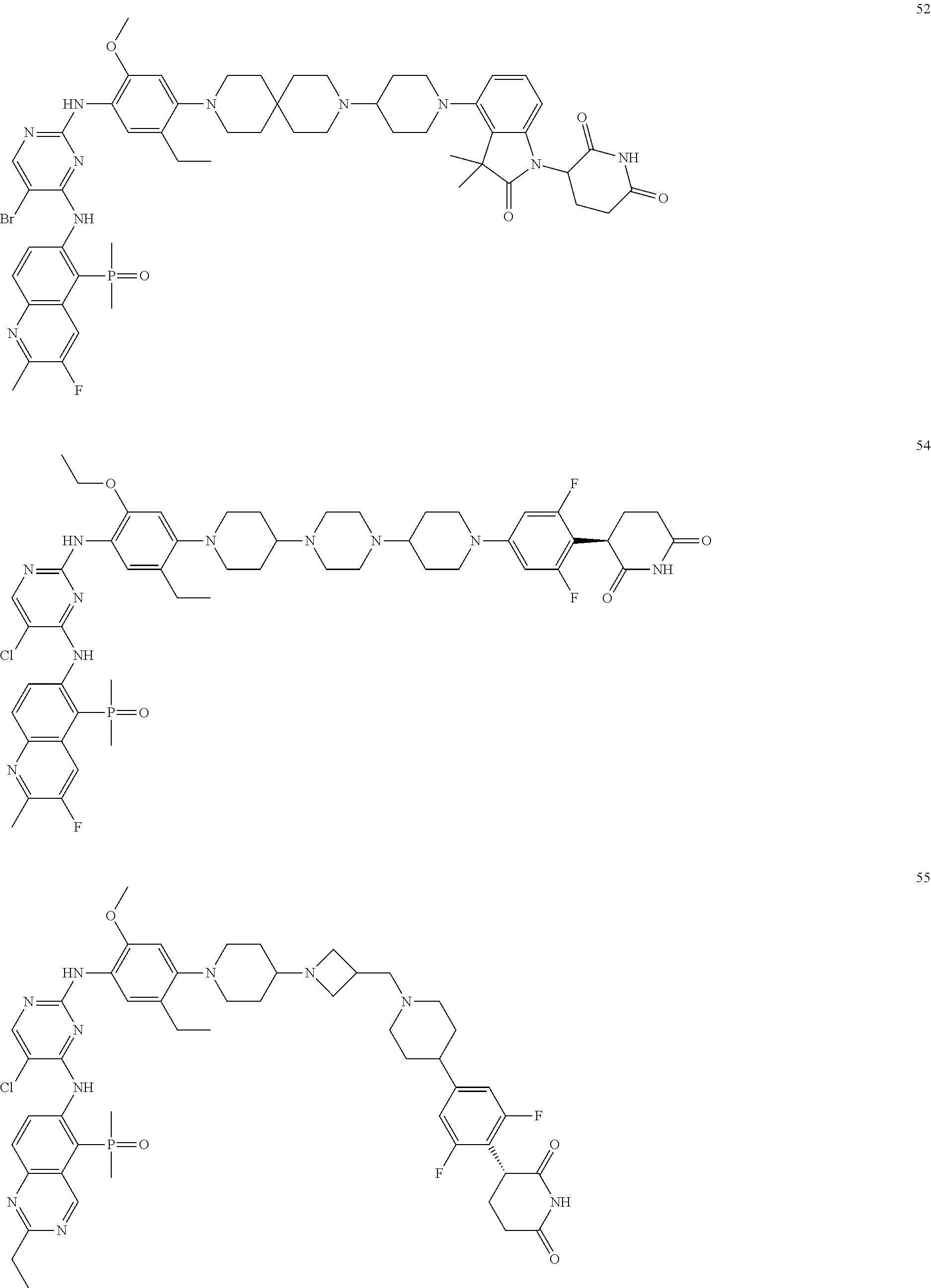 [SMILES: CCc1cc(Nc2ncc(Br)c(Nc3ccc4nc(C)c(F)cc4c3P(C)(C)=O)n2)c(OC)cc1N1CCC2(CC1)CCN(C1CCN(c3cccc4c3C(C)(C)C(=O)N4C3CCC(=O)NC3=O)CC1)CC2 ; CCOc1cc(N2CCC(N3CCN(C4CCN(c5cc(F)c([C@H]6CCC(=O)NC6=O)c(F)c5)CC4)CC3)CC2)c(CC)cc1Nc1ncc(Cl)c(Nc2ccc3nc(C)c(F)cc3c2P(C)(C)=O)n1 ; CCc1ncc2c(P(C)(C)=O)c(Nc3nc(Nc4cc(CC)c(N5CCC(N6CC(CN7CCC(c8cc(F)c([C@H]9CCC(=O)NC9=O)c(F)c8)CC7)C6)CC5)cc4OC)ncc3Cl)ccc2n1]

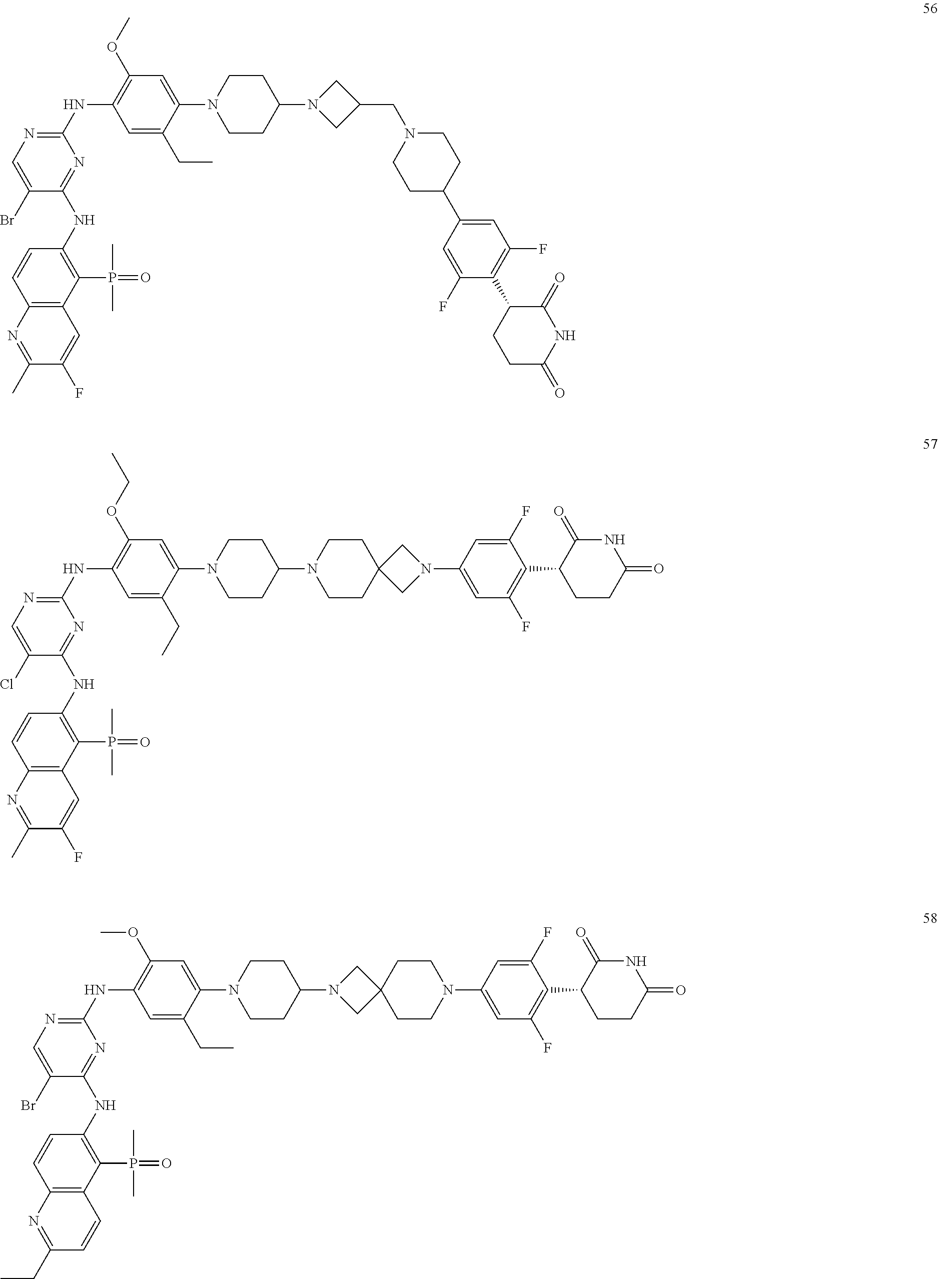

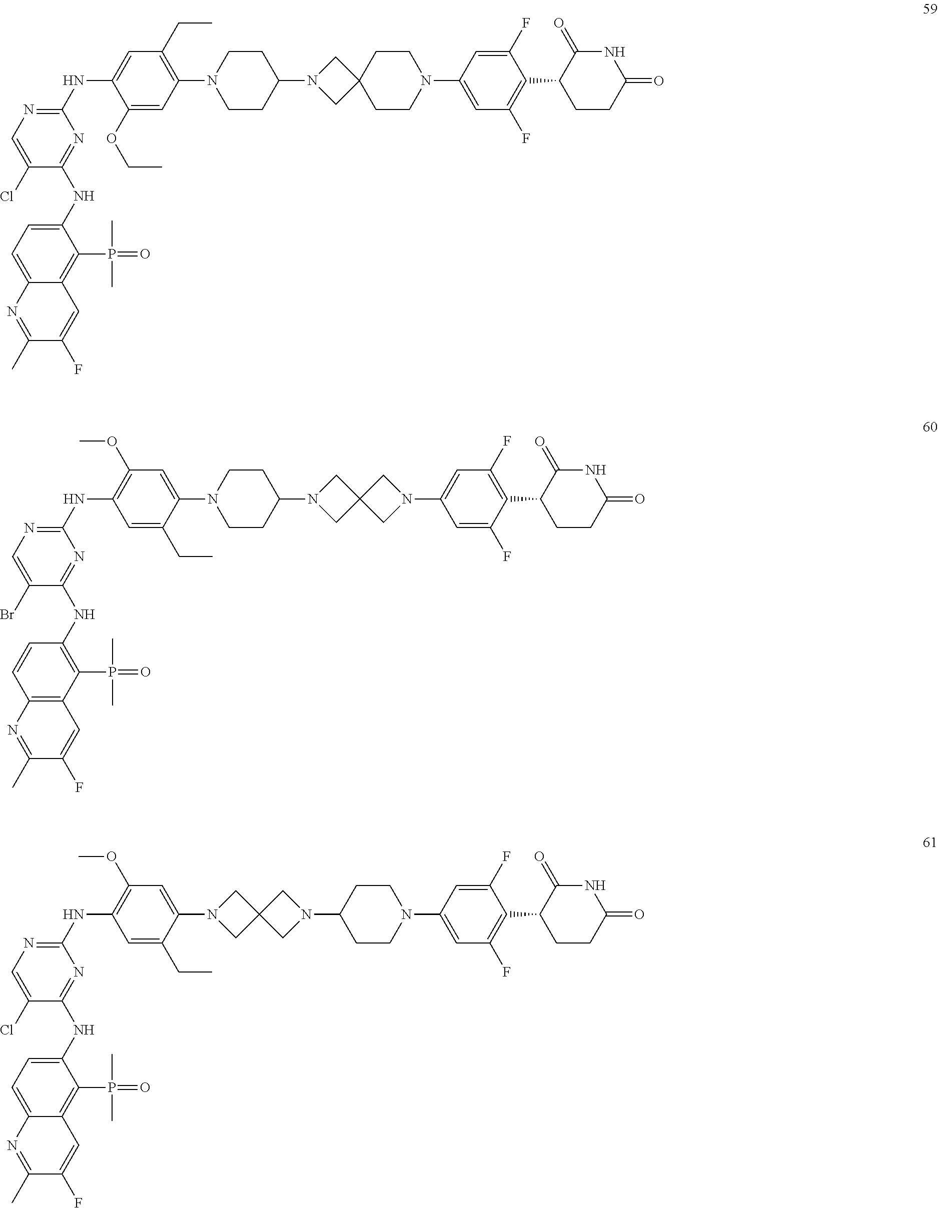 [SMILES: CCOc1cc(N2CCC(N3CC4(CCN(c5cc(F)c([C@H]6CCC(=O)NC6=O)c(F)c5)CC4)C3)CC2)c(CC)cc1Nc1ncc(Cl)c(Nc2ccc3nc(C)c(F)cc3c2P(C)(C)=O)n1 ; CCc1cc(Nc2ncc(Br)c(Nc3ccc4nc(C)c(F)cc4c3P(C)(C)=O)n2)c(OC)cc1N1CCC(N2CC3(CN(c4cc(F)c([C@H]5CCC(=O)NC5=O)c(F)c4)C3)C2)CC1 ; CCc1cc(Nc2ncc(Cl)c(Nc3ccc4nc(C)c(F)cc4c3P(C)(C)=O)n2)c(OC)cc1N1CC2(C1)CN(C1CCN(c3cc(F)c([C@H]4CCC(=O)NC4=O)c(F)c3)CC1)C2]

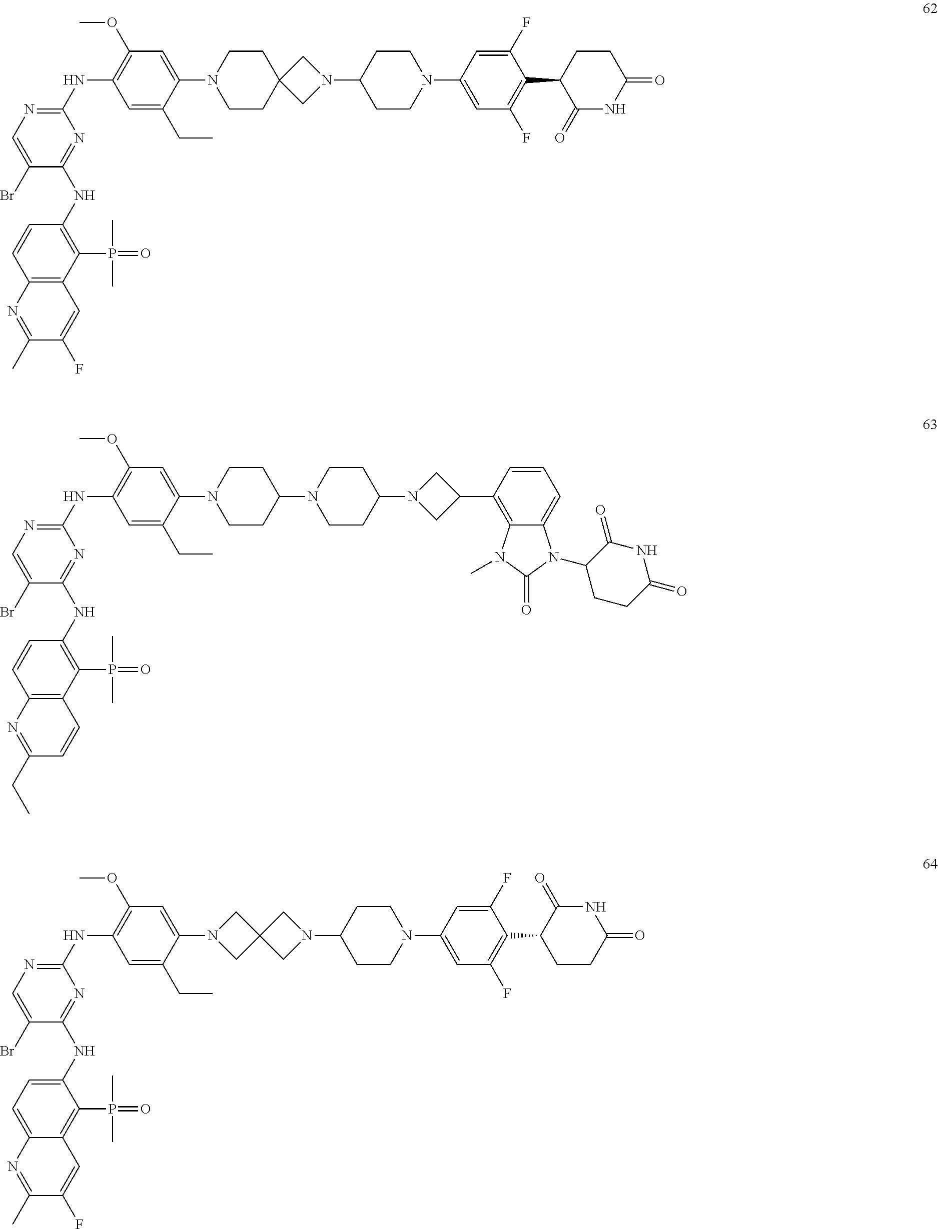

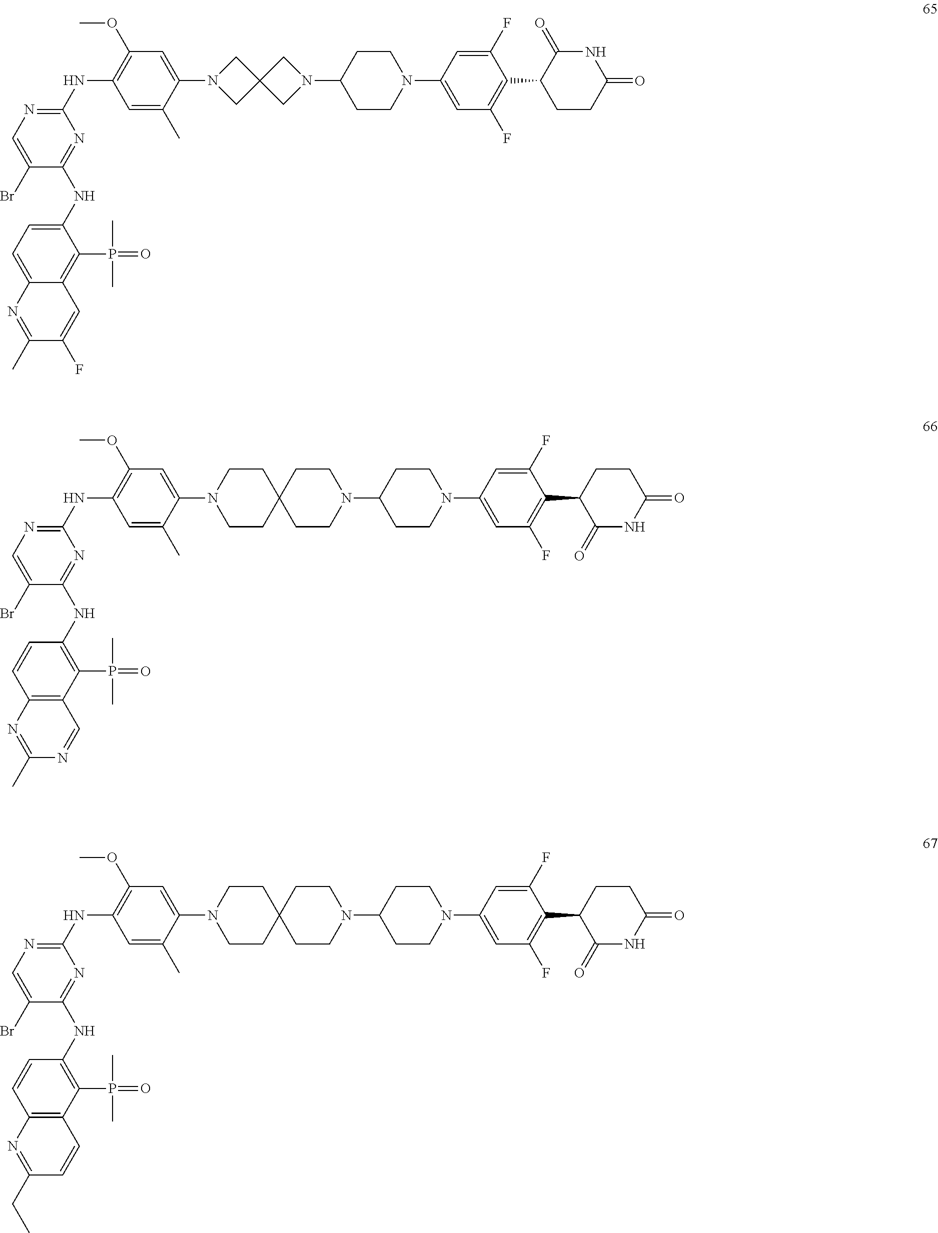 [SMILES: COc1cc(N2CC3(C2)CN(C2CCN(c4cc(F)c([C@H]5CCC(=O)NC5=O)c(F)c4)CC2)C3)c(C)cc1Nc1ncc(Br)c(Nc2ccc3nc(C)c(F)cc3c2P(C)(C)=O)n1 ; COc1cc(N2CCC3(CC2)CCN(C2CCN(c4cc(F)c([C@H]5CCC(=O)NC5=O)c(F)c4)CC2)CC3)c(C)cc1Nc1ncc(Br)c(Nc2ccc3nc(C)ncc3c2P(C)(C)=O)n1 ; CCc1ccc2c(P(C)(C)=O)c(Nc3nc(Nc4cc(C)c(N5CCC6(CC5)CCN(C5CCN(c7cc(F)c([C@H]8CCC(=O)NC8=O)c(F)c7)CC5)CC6)cc4OC)ncc3Br)ccc2n1]

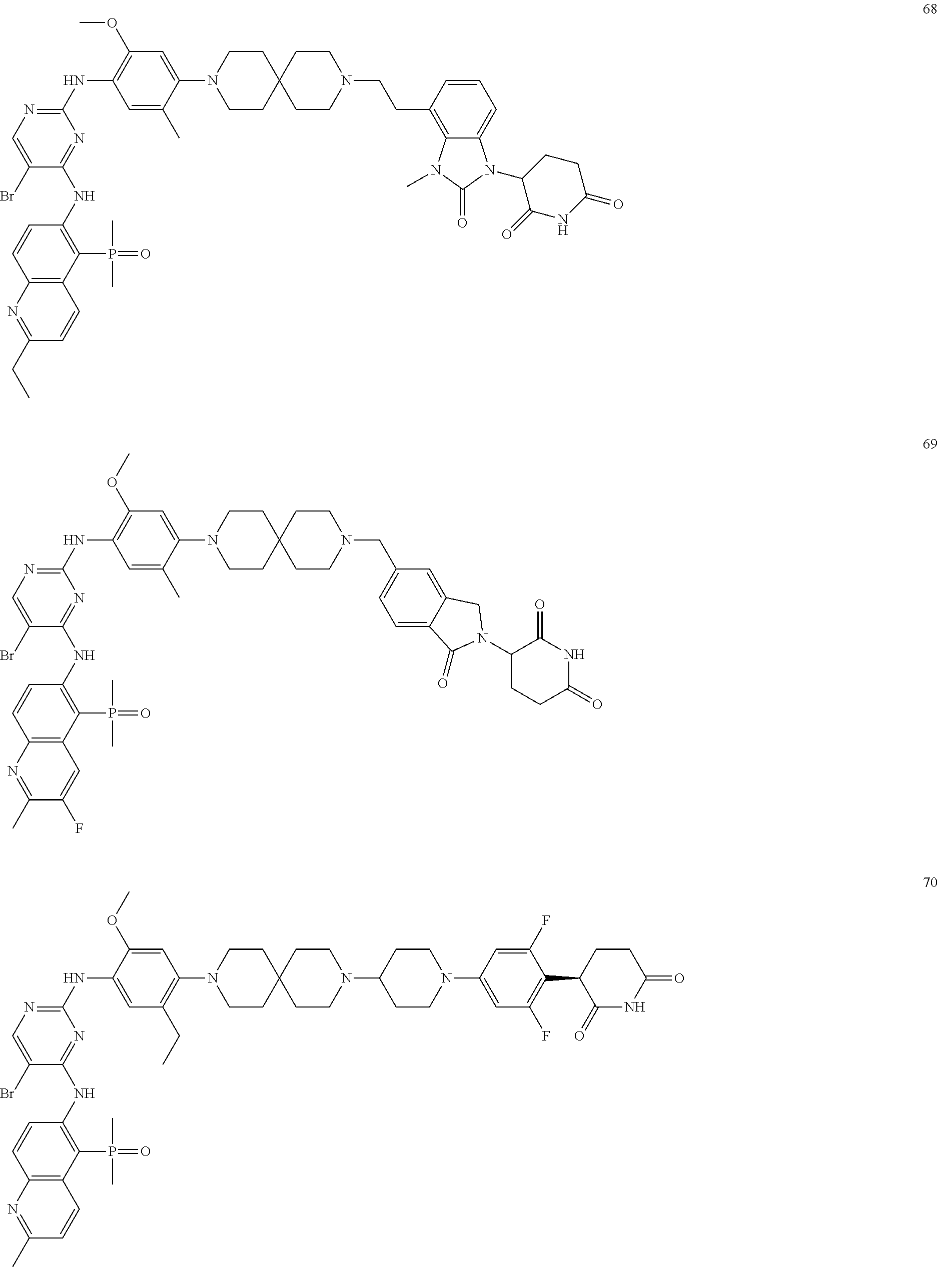

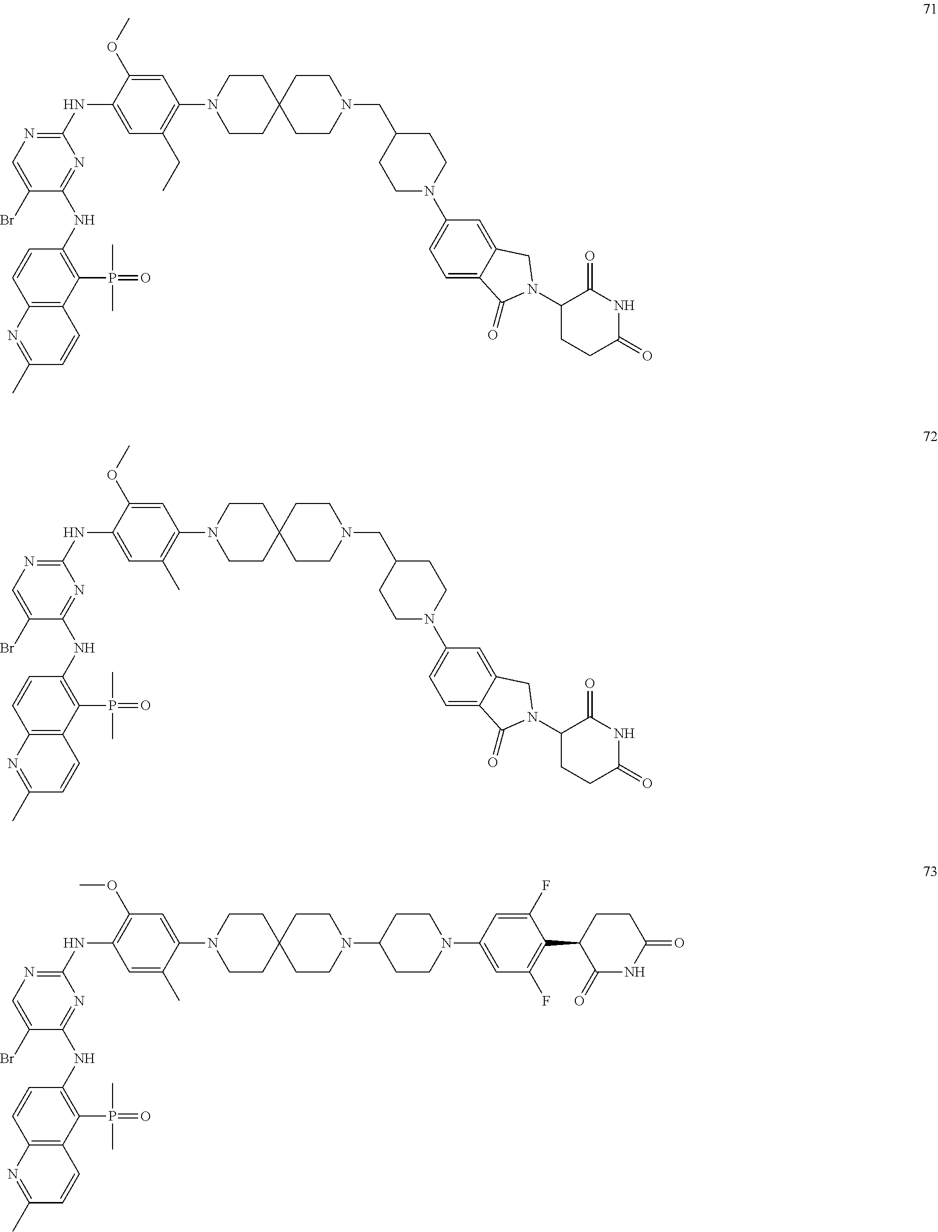 [SMILES: CCc1cc(Nc2ncc(Br)c(Nc3ccc4nc(C)ccc4c3P(C)(C)=O)n2)c(OC)cc1N1CCC2(CCN(CC3CCN(c4ccc5c(c4)CN(C4CCC(=O)NC4=O)C5=O)CC3)CC2)CC1 ; COc1cc(N2CCC3(CCN(CC4CCN(c5ccc6c(c5)CN(C5CCC(=O)NC5=O)C6=O)CC4)CC3)CC2)c(C)cc1Nc1ncc(Br)c(Nc2ccc3nc(C)ccc3c2P(C)(C)=O)n1 ; COc1cc(N2CCC3(CC2)CCN(C2CCN(c4cc(F)c([C@H]5CCC(=O)NC5=O)c(F)c4)CC2)CC3)c(C)cc1Nc1ncc(Br)c(Nc2ccc3nc(C)ccc3c2P(C)(C)=O)n1]

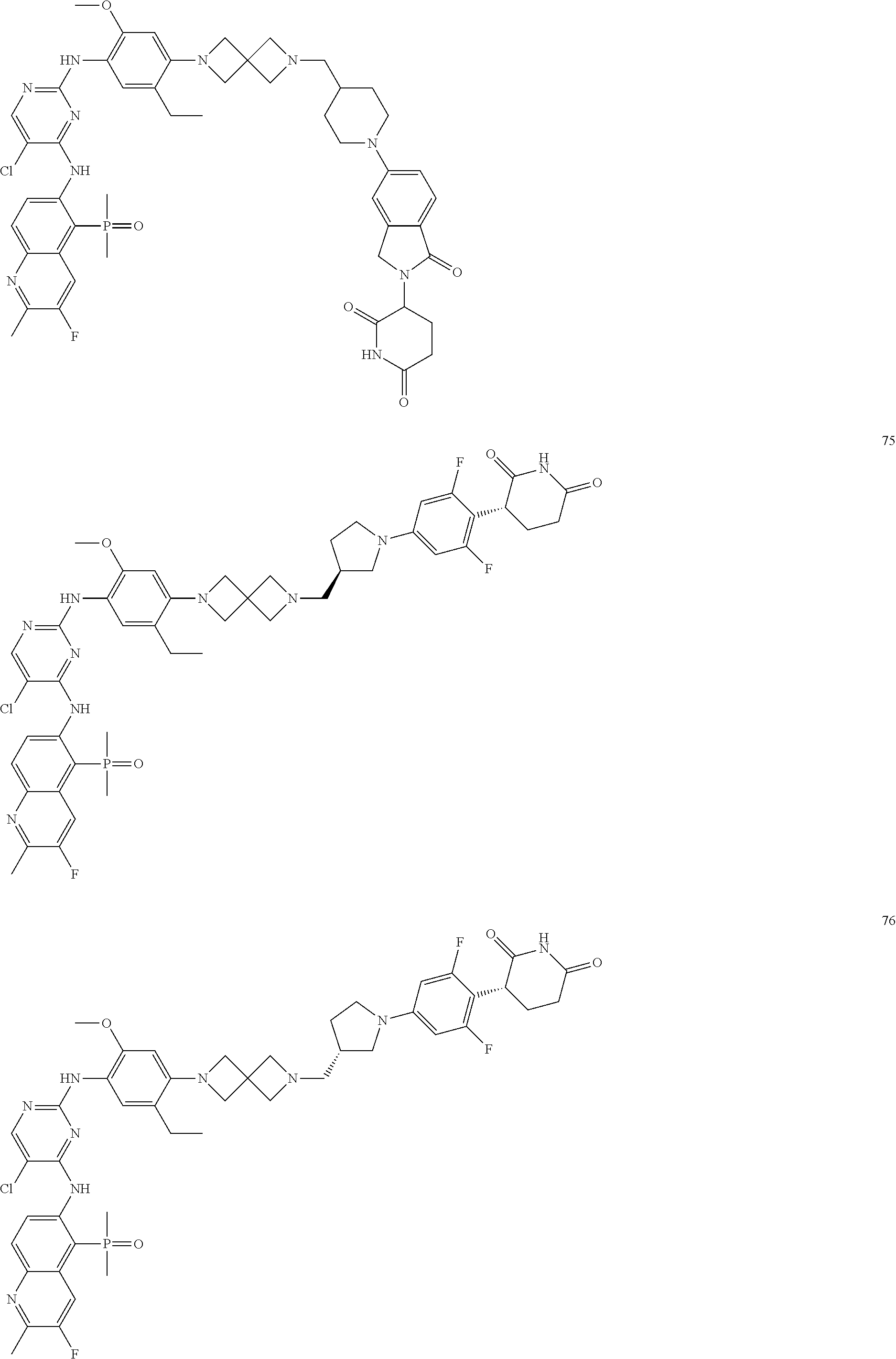 [SMILES: CCc1cc(Nc2ncc(Cl)c(Nc3ccc4nc(C)c(F)cc4c3P(C)(C)=O)n2)c(OC)cc1N1CC2(CN(CC3CCN(c4ccc5c(c4)CN(C4CCC(=O)NC4=O)C5=O)CC3)C2)C1 ; CCc1cc(Nc2ncc(Cl)c(Nc3ccc4nc(C)c(F)cc4c3P(C)(C)=O)n2)c(OC)cc1N1CC2(CN(C[C@H]3CCN(c4cc(F)c([C@H]5CCC(=O)NC5=O)c(F)c4)C3)C2)C1 ; CCc1cc(Nc2ncc(Cl)c(Nc3ccc4nc(C)c(F)cc4c3P(C)(C)=O)n2)c(OC)cc1N1CC2(CN(C[C@@H]3CCN(c4cc(F)c([C@H]5CCC(=O)NC5=O)c(F)c4)C3)C2)C1]

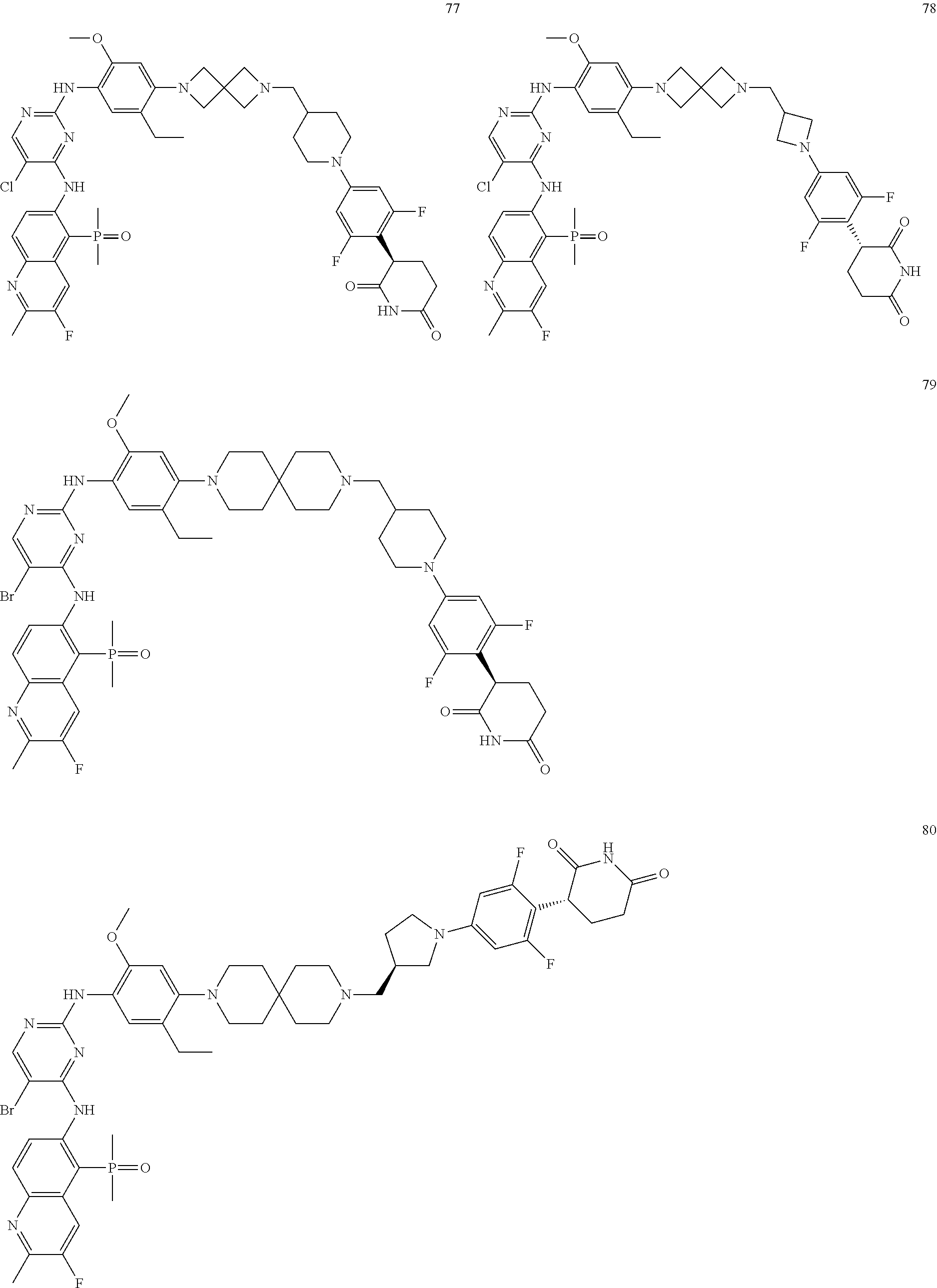

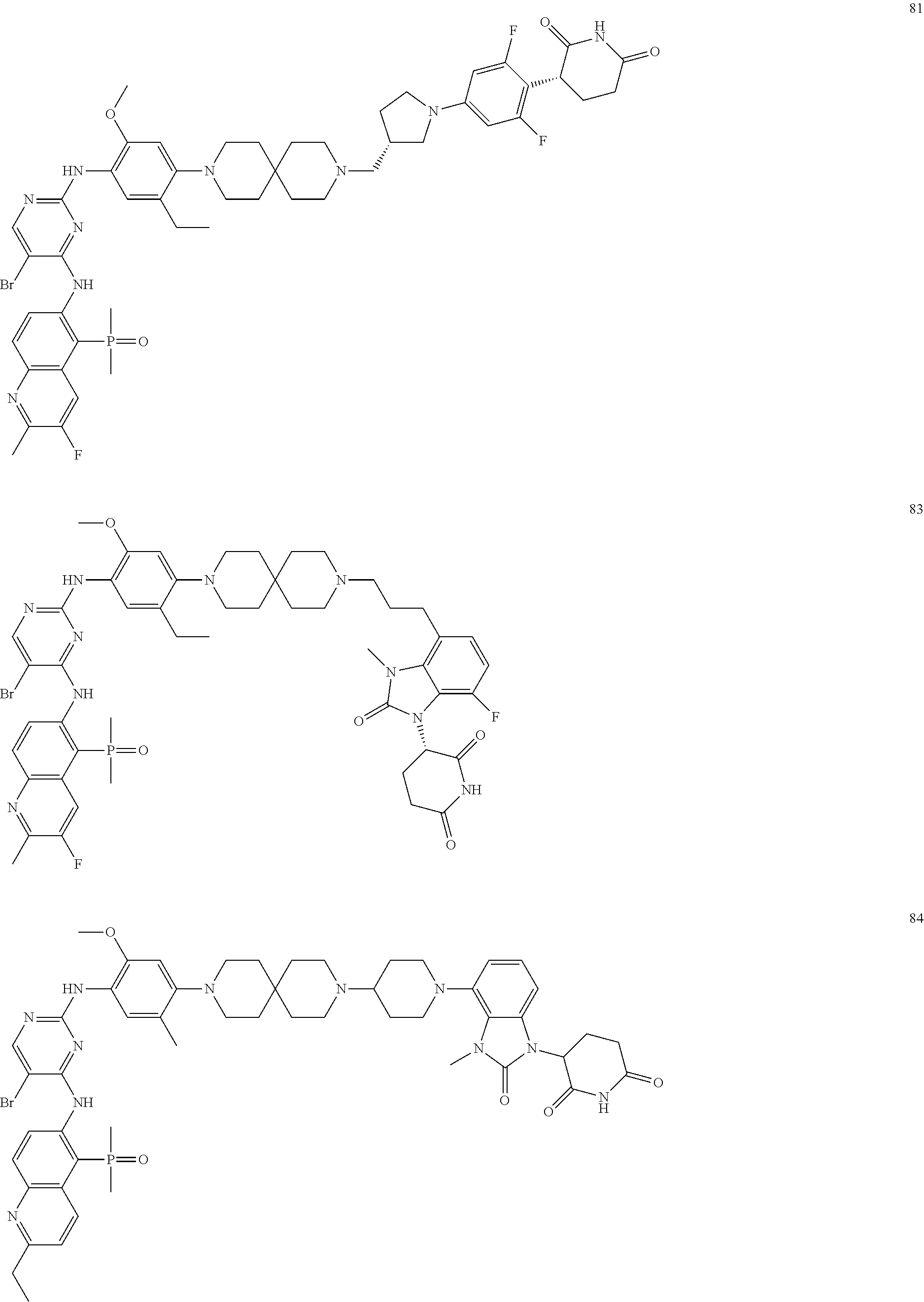

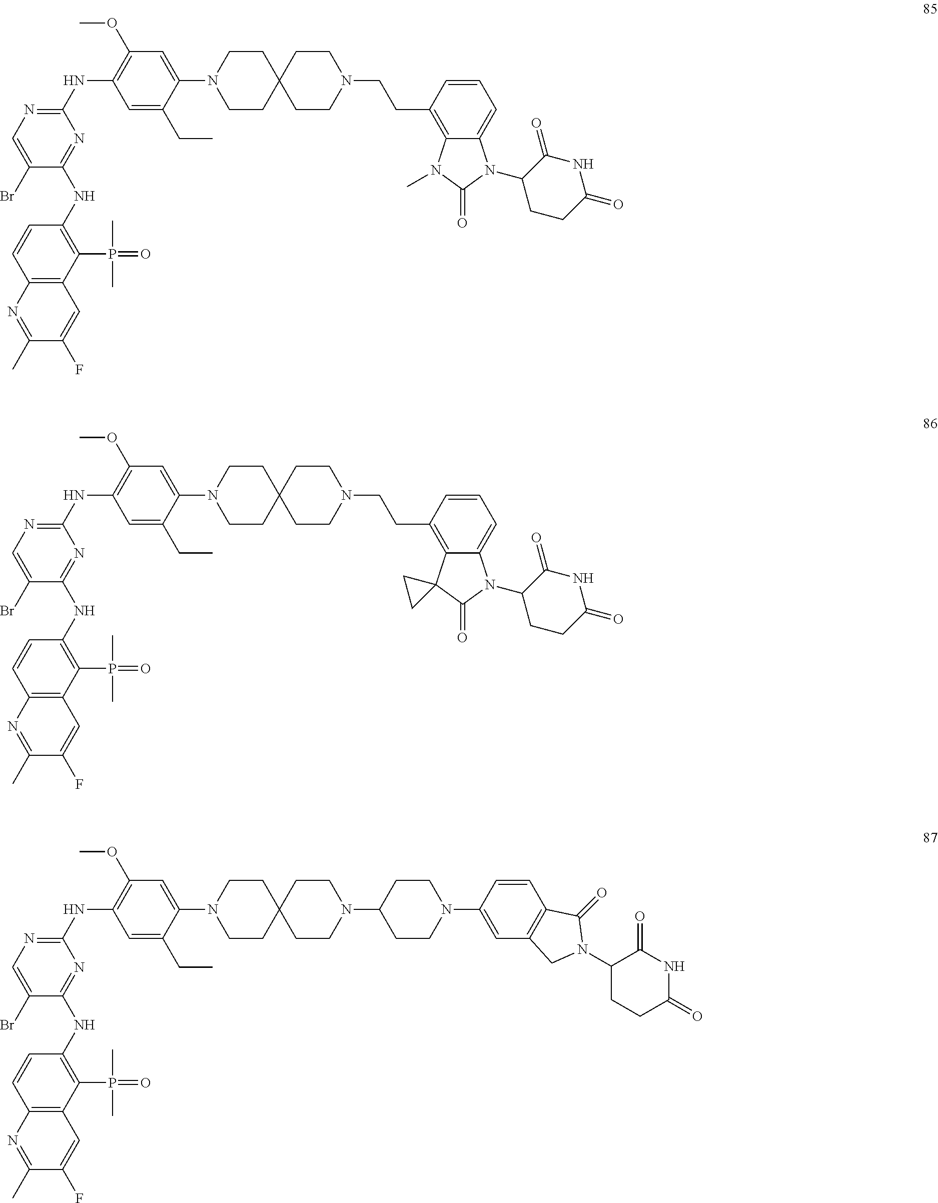

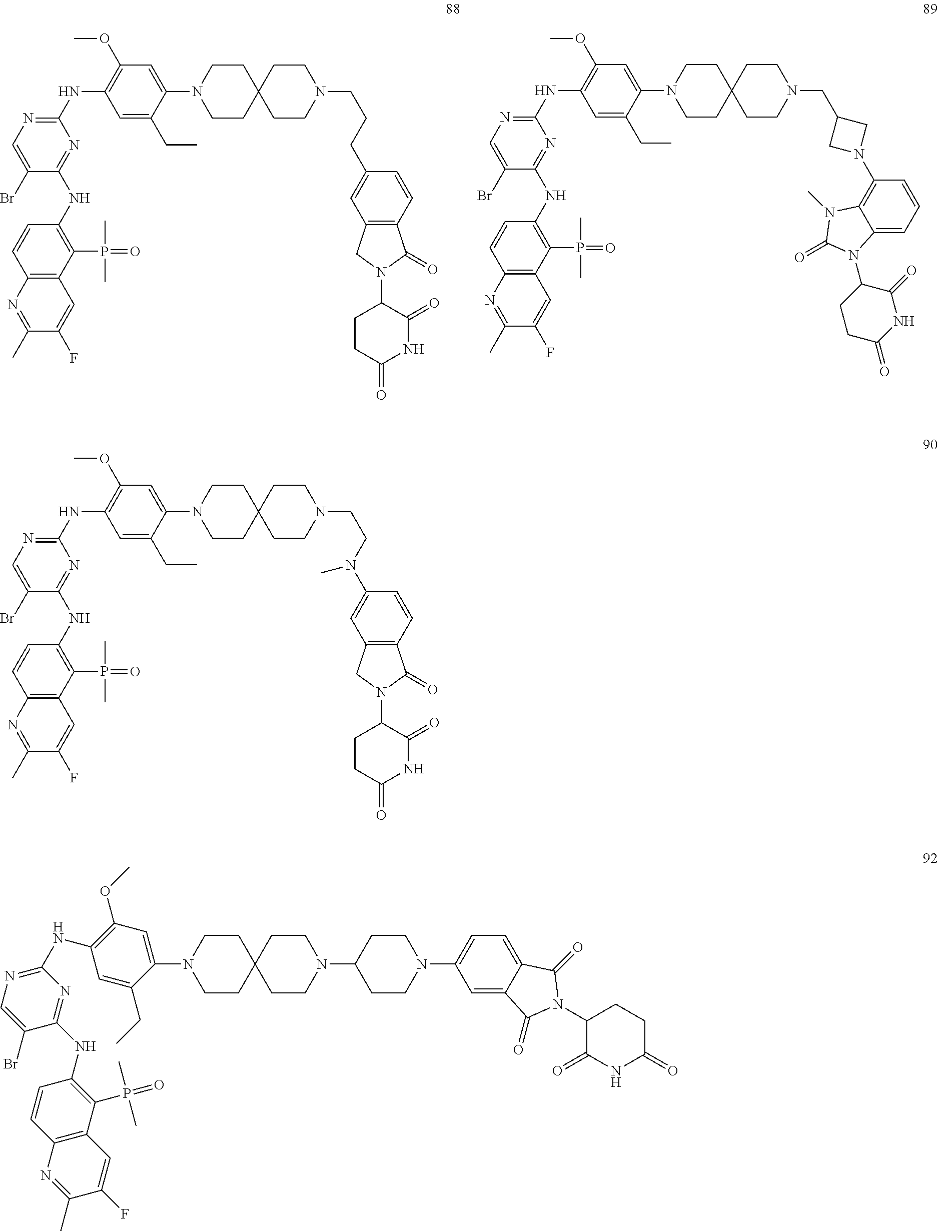

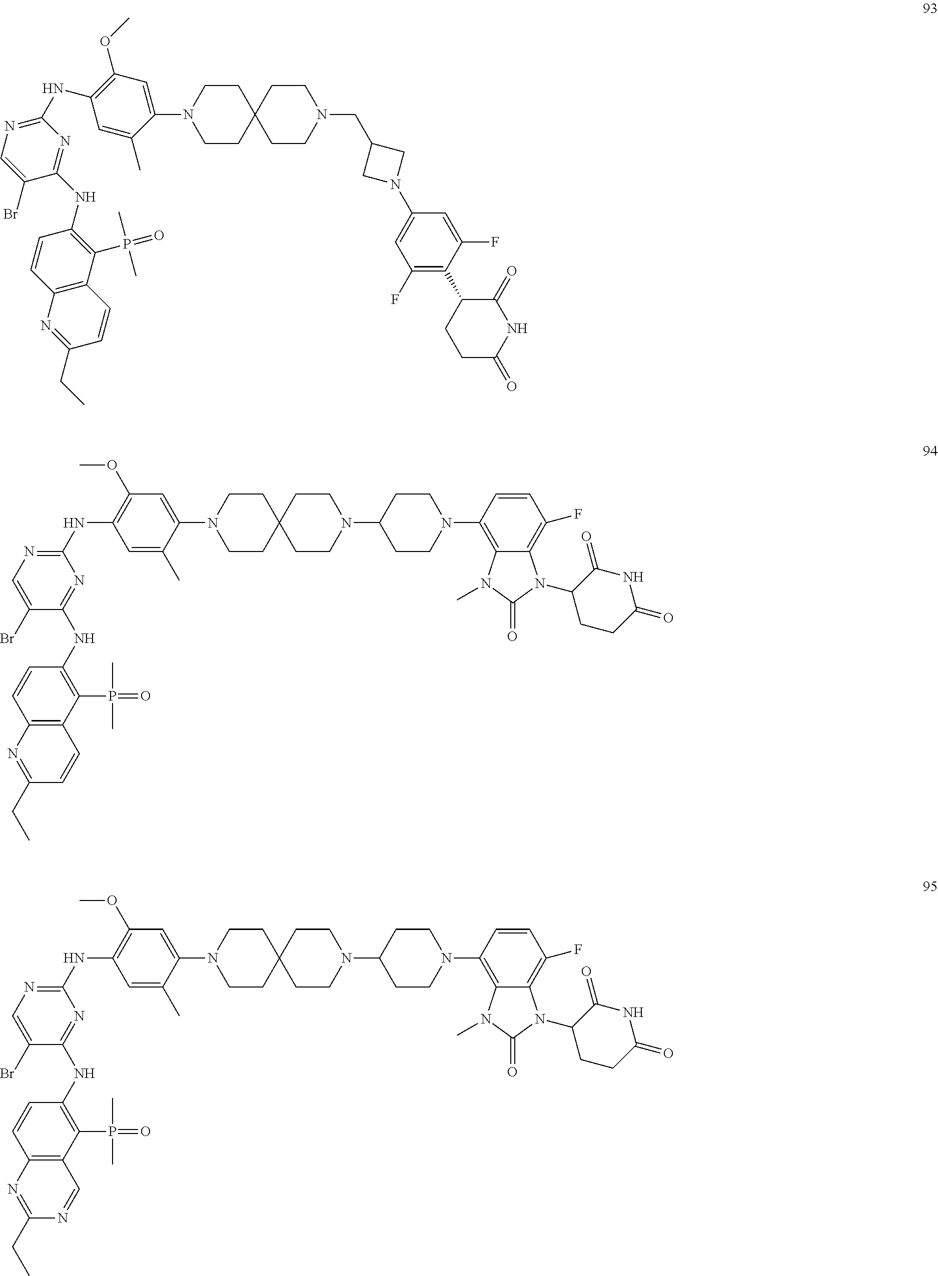

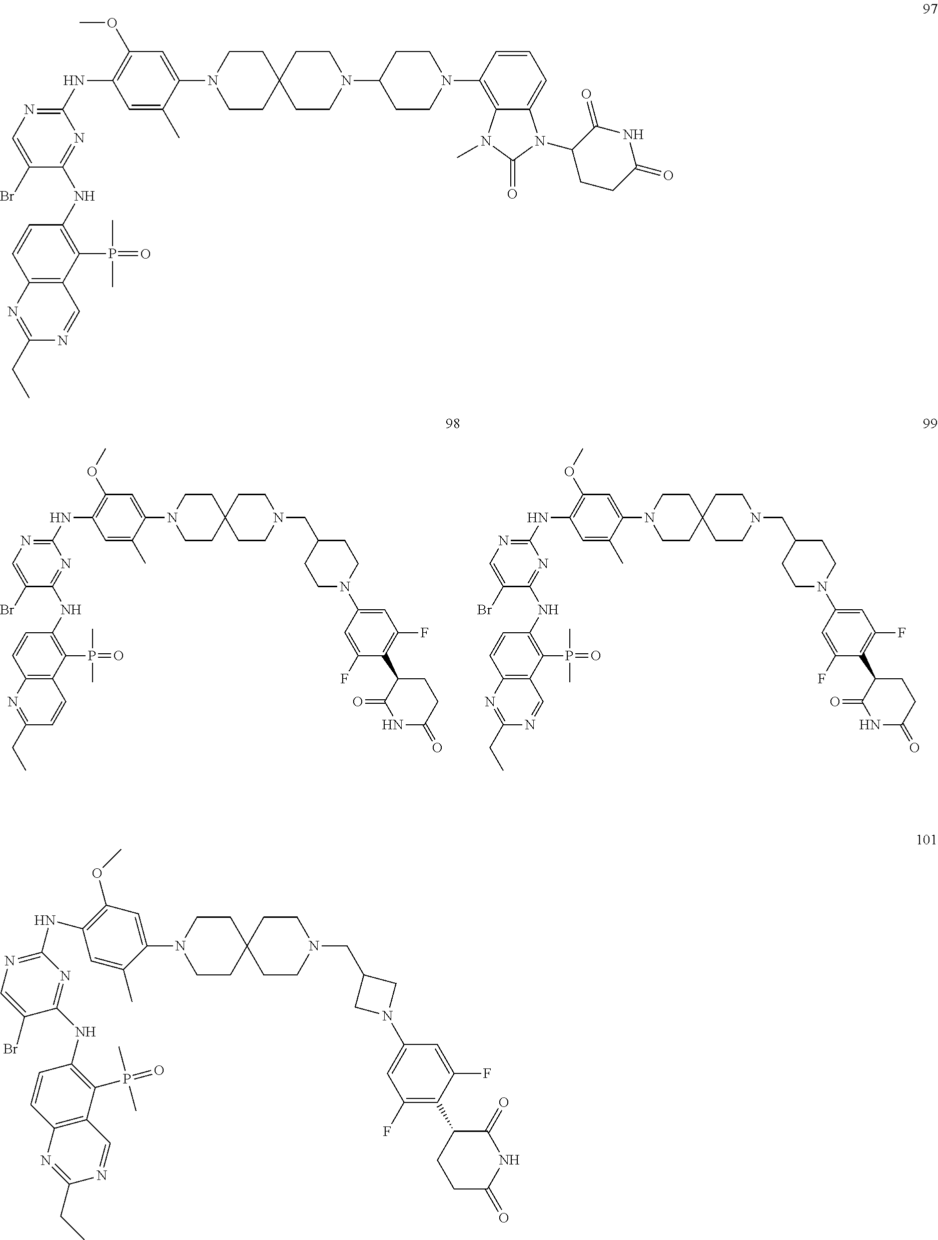

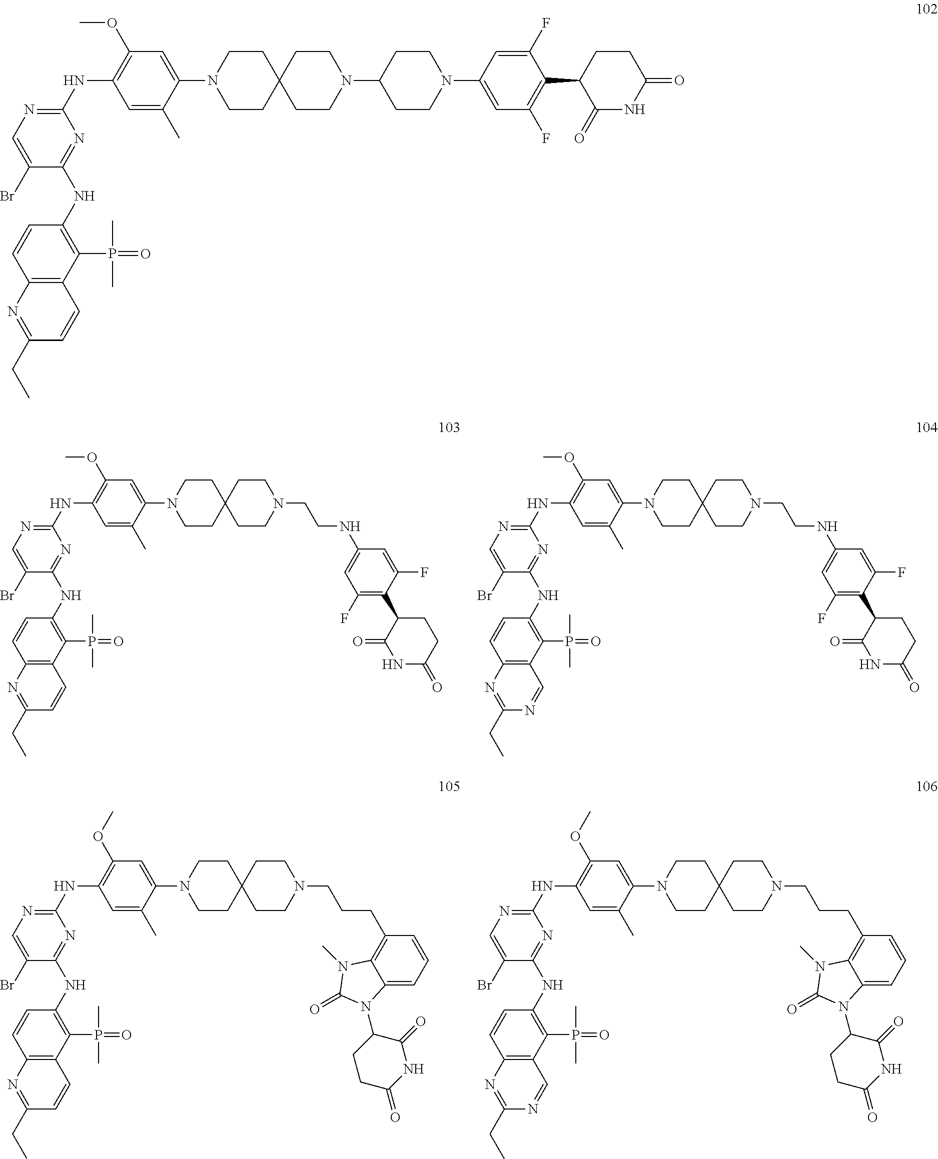

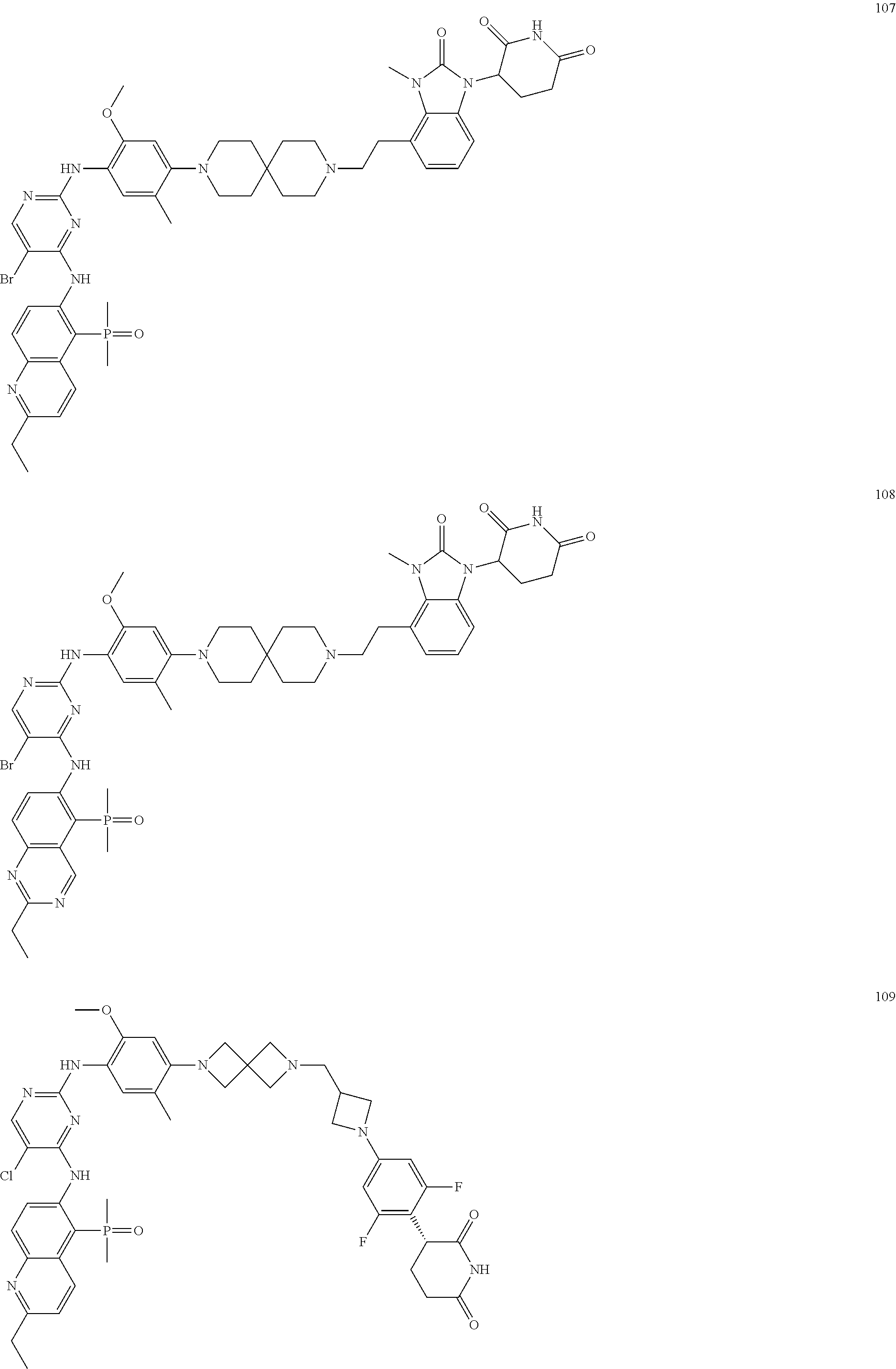

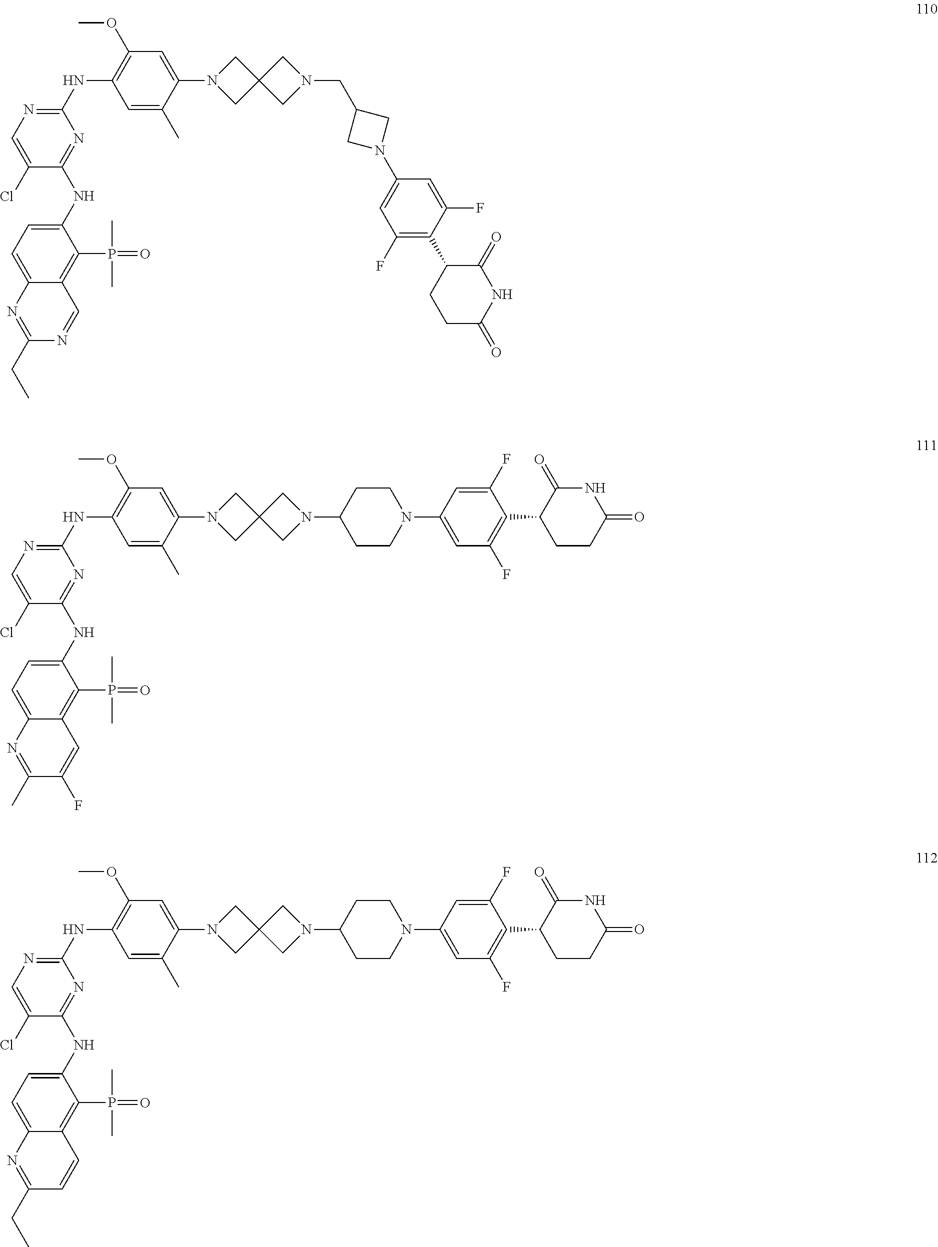 [SMILES: CCc1ncc2c(P(C)(C)=O)c(Nc3nc(Nc4cc(C)c(N5CC6(CN(CC7CN(c8cc(F)c([C@H]9CCC(=O)NC9=O)c(F)c8)C7)C6)C5)cc4OC)ncc3Cl)ccc2n1 ; COc1cc(N2CC3(C2)CN(C2CCN(c4cc(F)c([C@H]5CCC(=O)NC5=O)c(F)c4)CC2)C3)c(C)cc1Nc1ncc(Cl)c(Nc2ccc3nc(C)c(F)cc3c2P(C)(C)=O)n1 ; CCc1ccc2c(P(C)(C)=O)c(Nc3nc(Nc4cc(C)c(N5CC6(C5)CN(C5CCN(c7cc(F)c([C@H]8CCC(=O)NC8=O)c(F)c7)CC5)C6)cc4OC)ncc3Cl)ccc2n1]

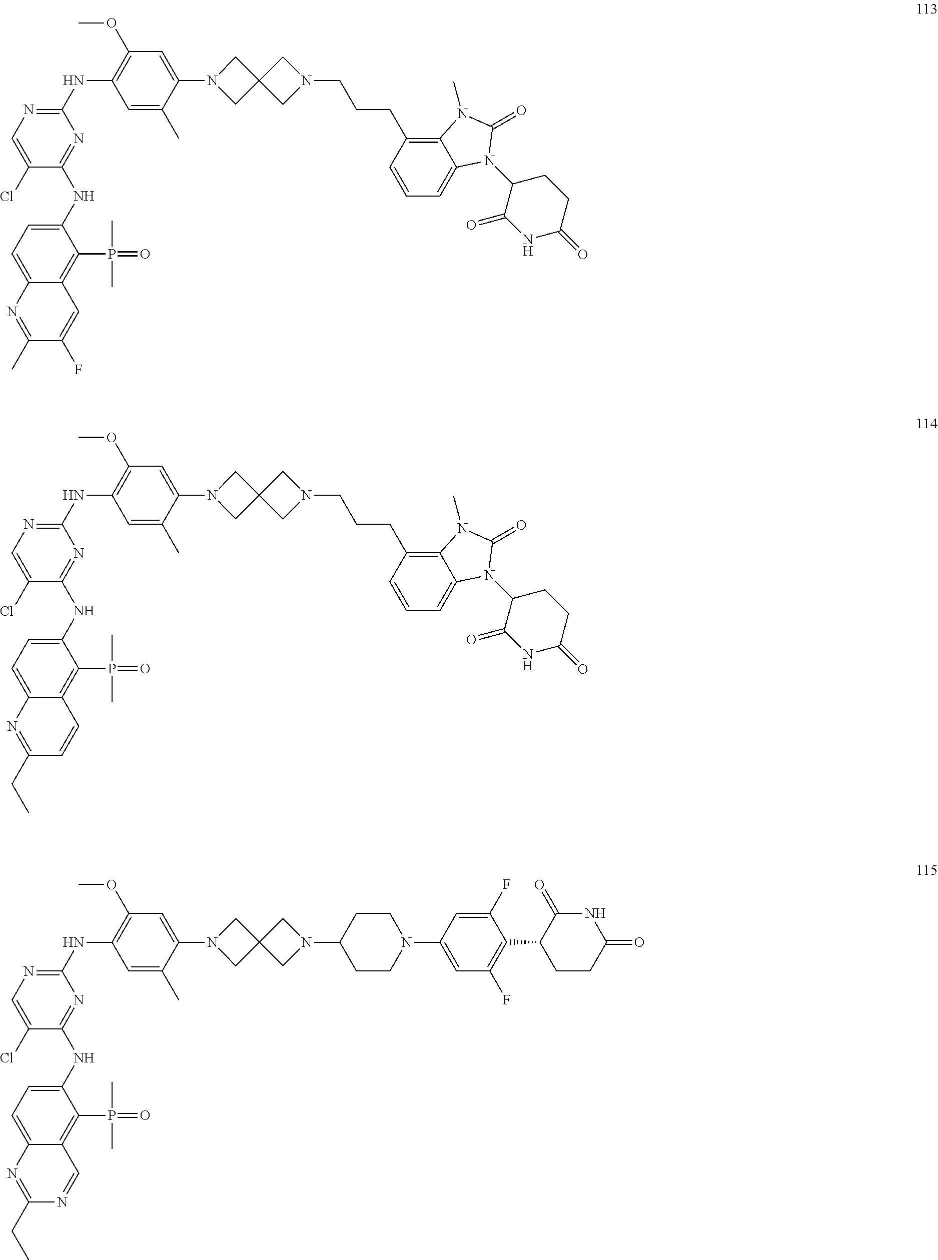 [SMILES: COc1cc(N2CC3(CN(CCCc4cccc5c4n(C)c(=O)n5C4CCC(=O)NC4=O)C3)C2)c(C)cc1Nc1ncc(Cl)c(Nc2ccc3nc(C)c(F)cc3c2P(C)(C)=O)n1 ; CCc1ccc2c(P(C)(C)=O)c(Nc3nc(Nc4cc(C)c(N5CC6(CN(CCCc7cccc8c7n(C)c(=O)n8C7CCC(=O)NC7=O)C6)C5)cc4OC)ncc3Cl)ccc2n1 ; CCc1ncc2c(P(C)(C)=O)c(Nc3nc(Nc4cc(C)c(N5CC6(C5)CN(C5CCN(c7cc(F)c([C@H]8CCC(=O)NC8=O)c(F)c7)CC5)C6)cc4OC)ncc3Cl)ccc2n1]

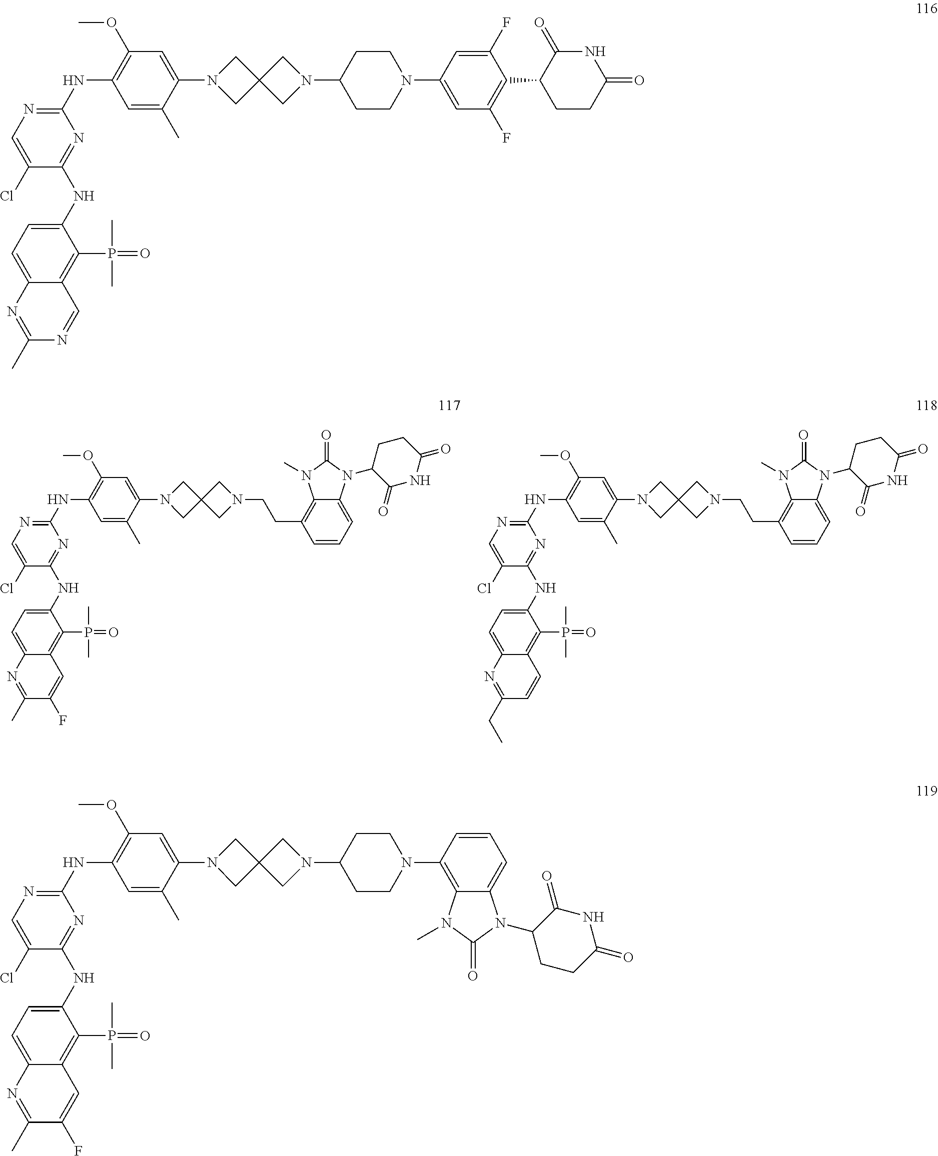 [SMILES: COc1cc(N2CC3(C2)CN(C2CCN(c4cc(F)c([C@H]5CCC(=O)NC5=O)c(F)c4)CC2)C3)c(C)cc1Nc1ncc(Cl)c(Nc2ccc3nc(C)ncc3c2P(C)(C)=O)n1 ; COc1cc(N2CC3(CN(CCc4cccc5c4n(C)c(=O)n5C4CCC(=O)NC4=O)C3)C2)c(C)cc1Nc1ncc(Cl)c(Nc2ccc3nc(C)c(F)cc3c2P(C)(C)=O)n1 ; CCc1ccc2c(P(C)(C)=O)c(Nc3nc(Nc4cc(C)c(N5CC6(CN(CCc7cccc8c7n(C)c(=O)n8C7CCC(=O)NC7=O)C6)C5)cc4OC)ncc3Cl)ccc2n1 ; COc1cc(N2CC3(C2)CN(C2CCN(c4cccc5c4n(C)c(=O)n5C4CCC(=O)NC4=O)CC2)C3)c(C)cc1Nc1ncc(Cl)c(Nc2ccc3nc(C)c(F)cc3c2P(C)(C)=O)n1]

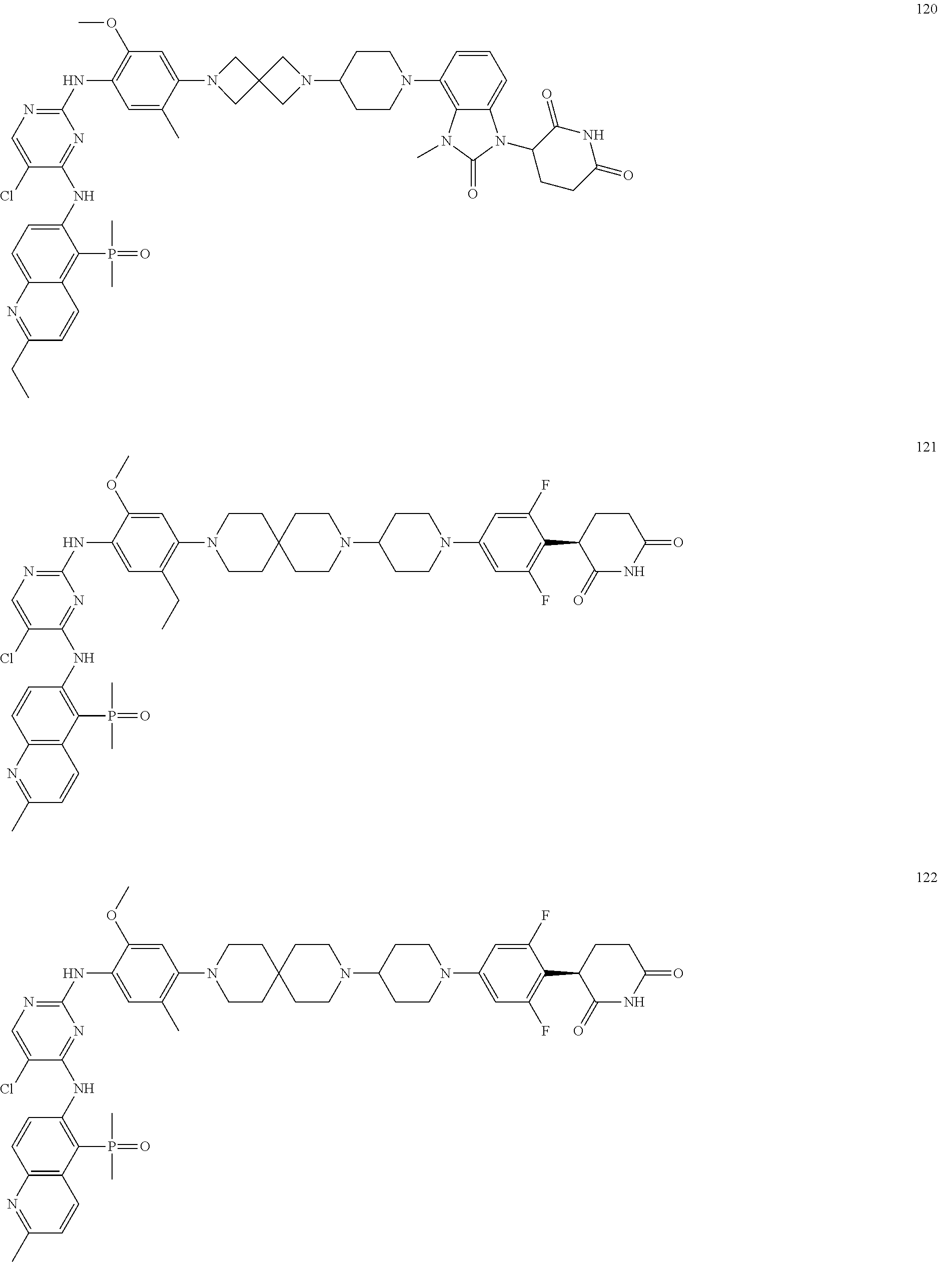 [SMILES: CCc1ccc2c(P(C)(C)=O)c(Nc3nc(Nc4cc(C)c(N5CC6(C5)CN(C5CCN(c7cccc8c7n(C)c(=O)n8C7CCC(=O)NC7=O)CC5)C6)cc4OC)ncc3Cl)ccc2n1 ; CCc1cc(Nc2ncc(Cl)c(Nc3ccc4nc(C)ccc4c3P(C)(C)=O)n2)c(OC)cc1N1CCC2(CC1)CCN(C1CCN(c3cc(F)c([C@H]4CCC(=O)NC4=O)c(F)c3)CC1)CC2 ; COc1cc(N2CCC3(CC2)CCN(C2CCN(c4cc(F)c([C@H]5CCC(=O)NC5=O)c(F)c4)CC2)CC3)c(C)cc1Nc1ncc(Cl)c(Nc2ccc3nc(C)ccc3c2P(C)(C)=O)n1]

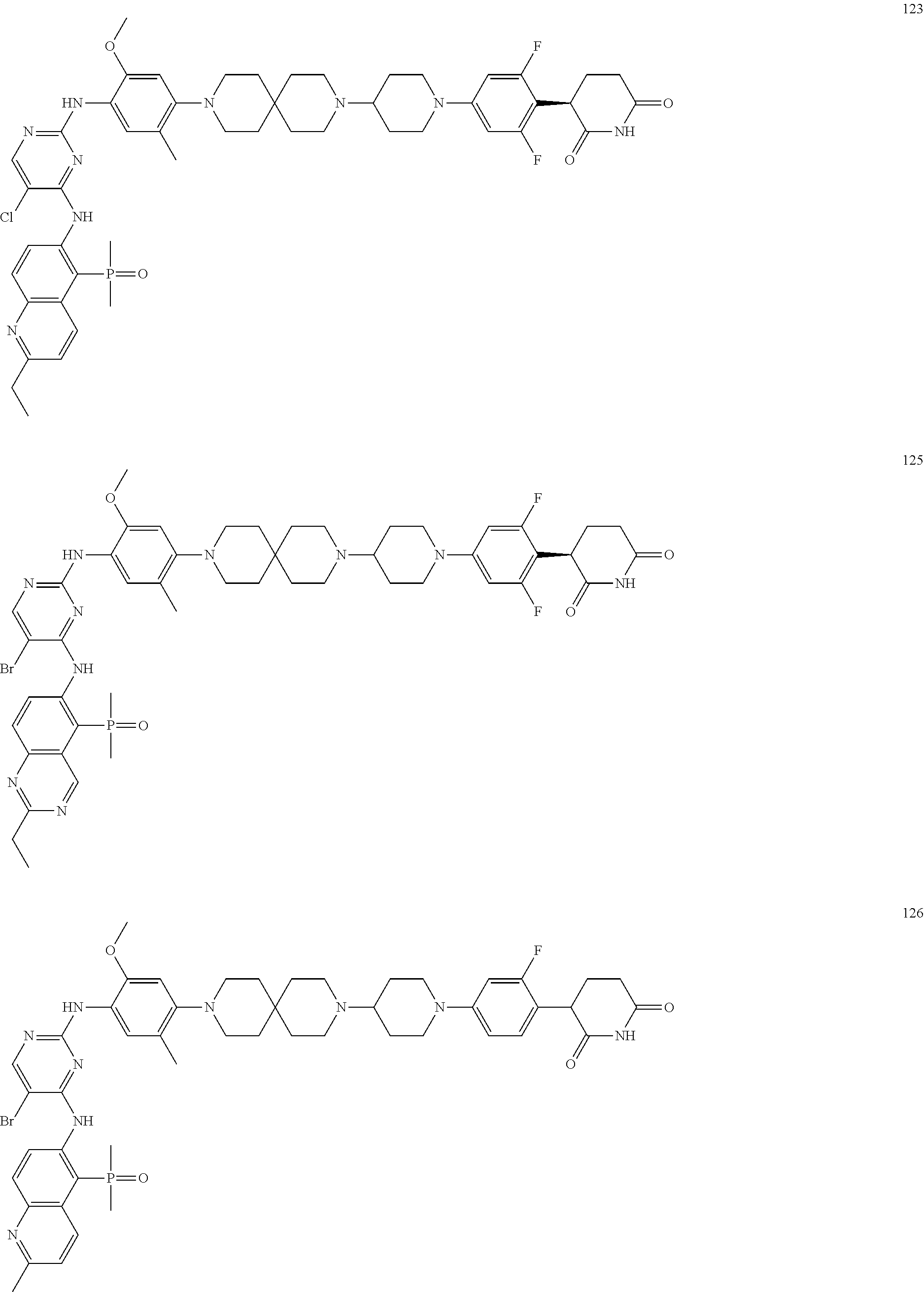 [SMILES: CCc1ccc2c(P(C)(C)=O)c(Nc3nc(Nc4cc(C)c(N5CCC6(CC5)CCN(C5CCN(c7cc(F)c([C@H]8CCC(=O)NC8=O)c(F)c7)CC5)CC6)cc4OC)ncc3Cl)ccc2n1 ; CCc1ncc2c(P(C)(C)=O)c(Nc3nc(Nc4cc(C)c(N5CCC6(CC5)CCN(C5CCN(c7cc(F)c([C@H]8CCC(=O)NC8=O)c(F)c7)CC5)CC6)cc4OC)ncc3Br)ccc2n1 ; COc1cc(N2CCC3(CC2)CCN(C2CCN(c4ccc(C5CCC(=O)NC5=O)c(F)c4)CC2)CC3)c(C)cc1Nc1ncc(Br)c(Nc2ccc3nc(C)ccc3c2P(C)(C)=O)n1]

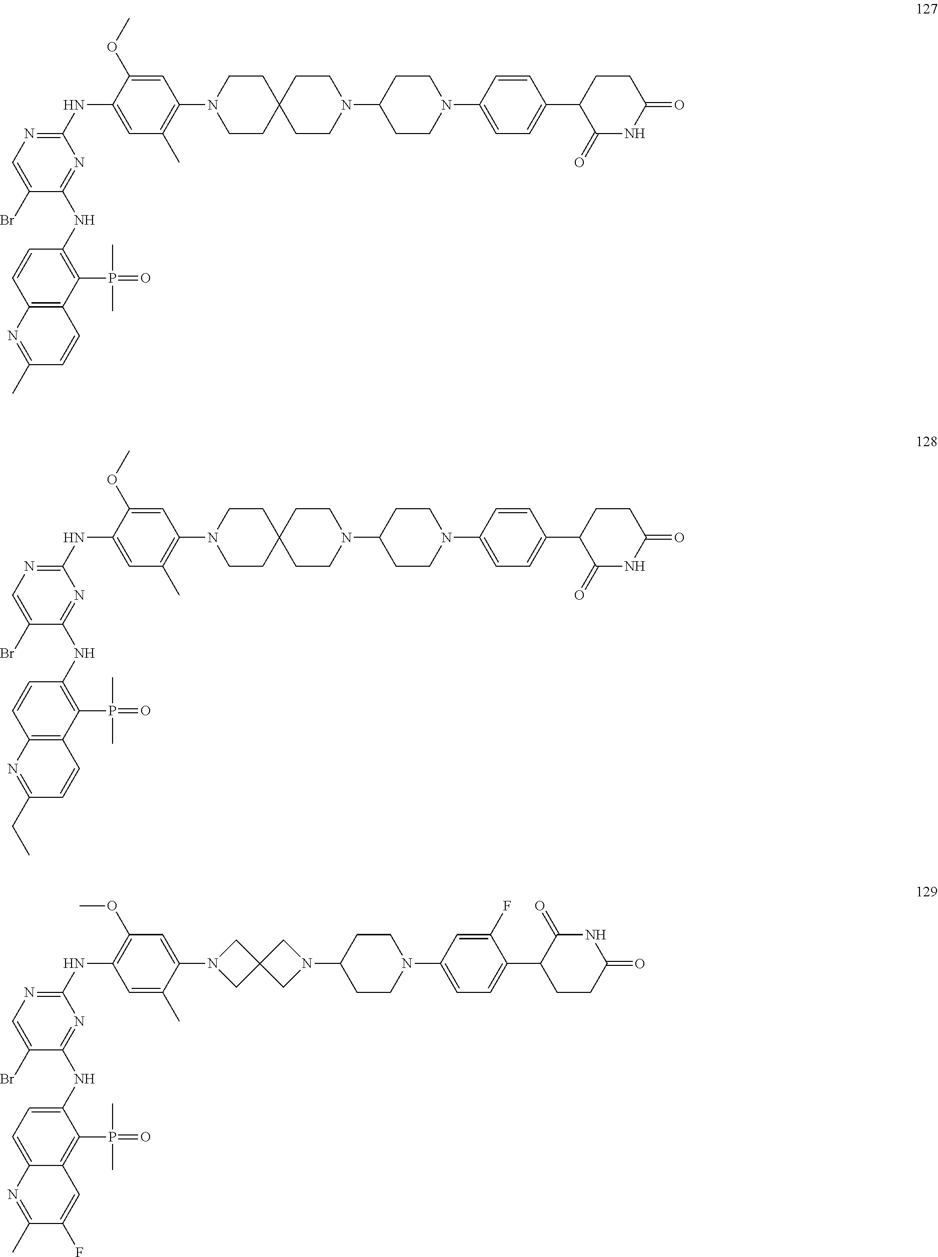 [SMILES: COc1cc(N2CCC3(CC2)CCN(C2CCN(c4ccc(C5CCC(=O)NC5=O)cc4)CC2)CC3)c(C)cc1Nc1ncc(Br)c(Nc2ccc3nc(C)ccc3c2P(C)(C)=O)n1 ; CCc1ccc2c(P(C)(C)=O)c(Nc3nc(Nc4cc(C)c(N5CCC6(CC5)CCN(C5CCN(c7ccc(C8CCC(=O)NC8=O)cc7)CC5)CC6)cc4OC)ncc3Br)ccc2n1 ; COc1cc(N2CC3(C2)CN(C2CCN(c4ccc(C5CCC(=O)NC5=O)c(F)c4)CC2)C3)c(C)cc1Nc1ncc(Br)c(Nc2ccc3nc(C)c(F)cc3c2P(C)(C)=O)n1]

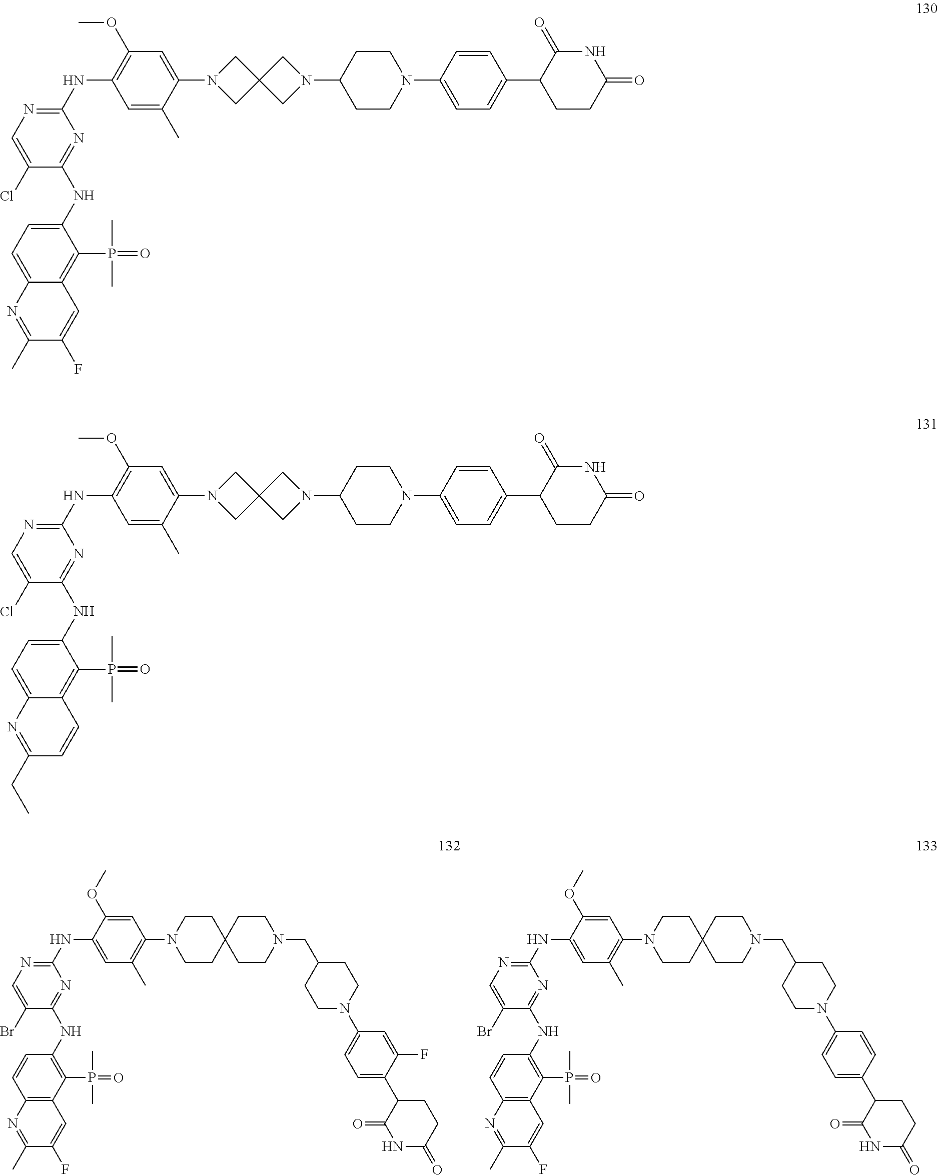 [SMILES: COc1cc(N2CC3(C2)CN(C2CCN(c4ccc(C5CCC(=O)NC5=O)cc4)CC2)C3)c(C)cc1Nc1ncc(Cl)c(Nc2ccc3nc(C)c(F)cc3c2P(C)(C)=O)n1 ; CCc1ccc2c(P(C)(C)=O)c(Nc3nc(Nc4cc(C)c(N5CC6(C5)CN(C5CCN(c7ccc(C8CCC(=O)NC8=O)cc7)CC5)C6)cc4OC)ncc3Cl)ccc2n1 ; COc1cc(N2CCC3(CCN(CC4CCN(c5ccc(C6CCC(=O)NC6=O)c(F)c5)CC4)CC3)CC2)c(C)cc1Nc1ncc(Br)c(Nc2ccc3nc(C)c(F)cc3c2P(C)(C)=O)n1 ; COc1cc(N2CCC3(CCN(CC4CCN(c5ccc(C6CCC(=O)NC6=O)cc5)CC4)CC3)CC2)c(C)cc1Nc1ncc(Br)c(Nc2ccc3nc(C)c(F)cc3c2P(C)(C)=O)n1]

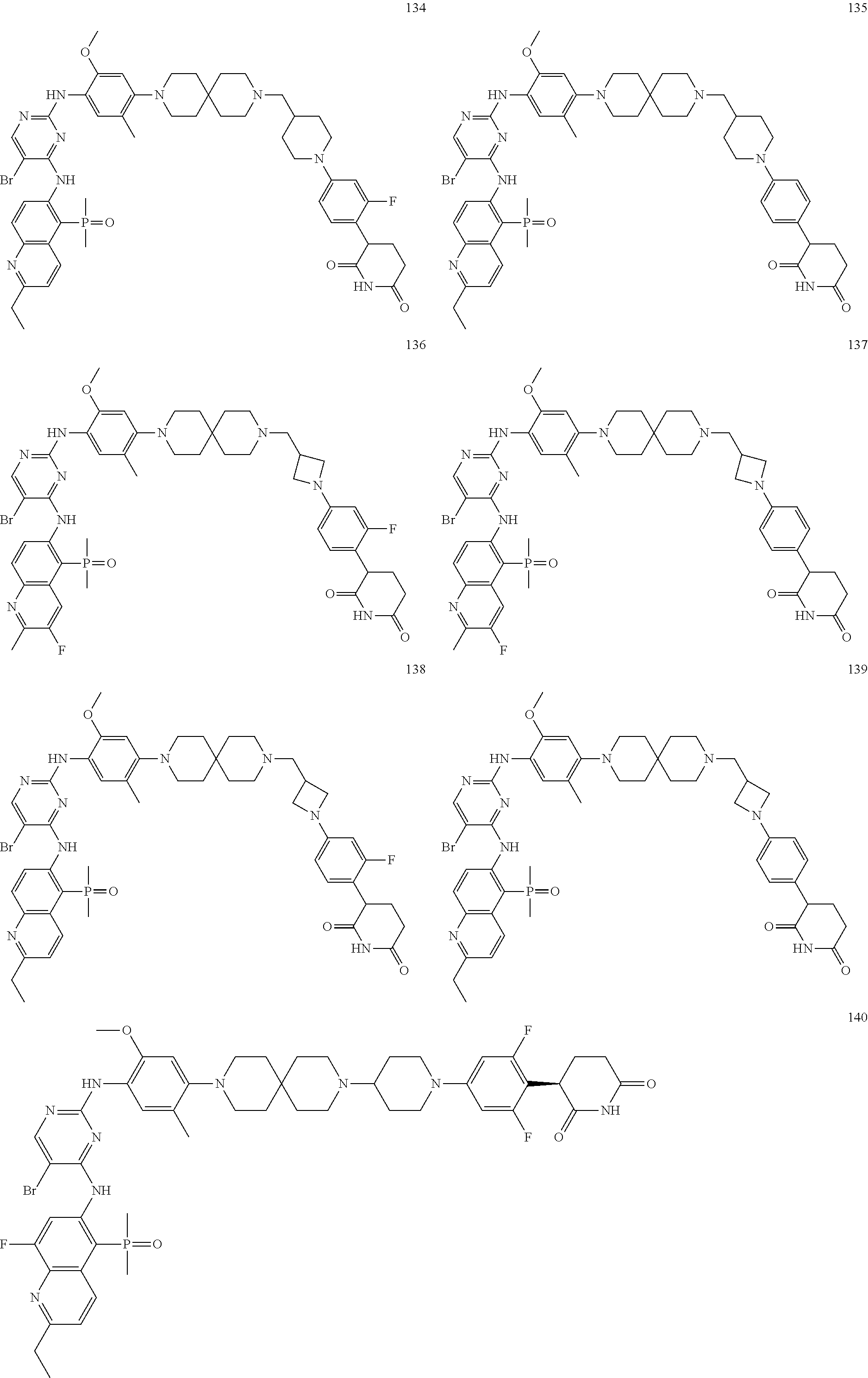

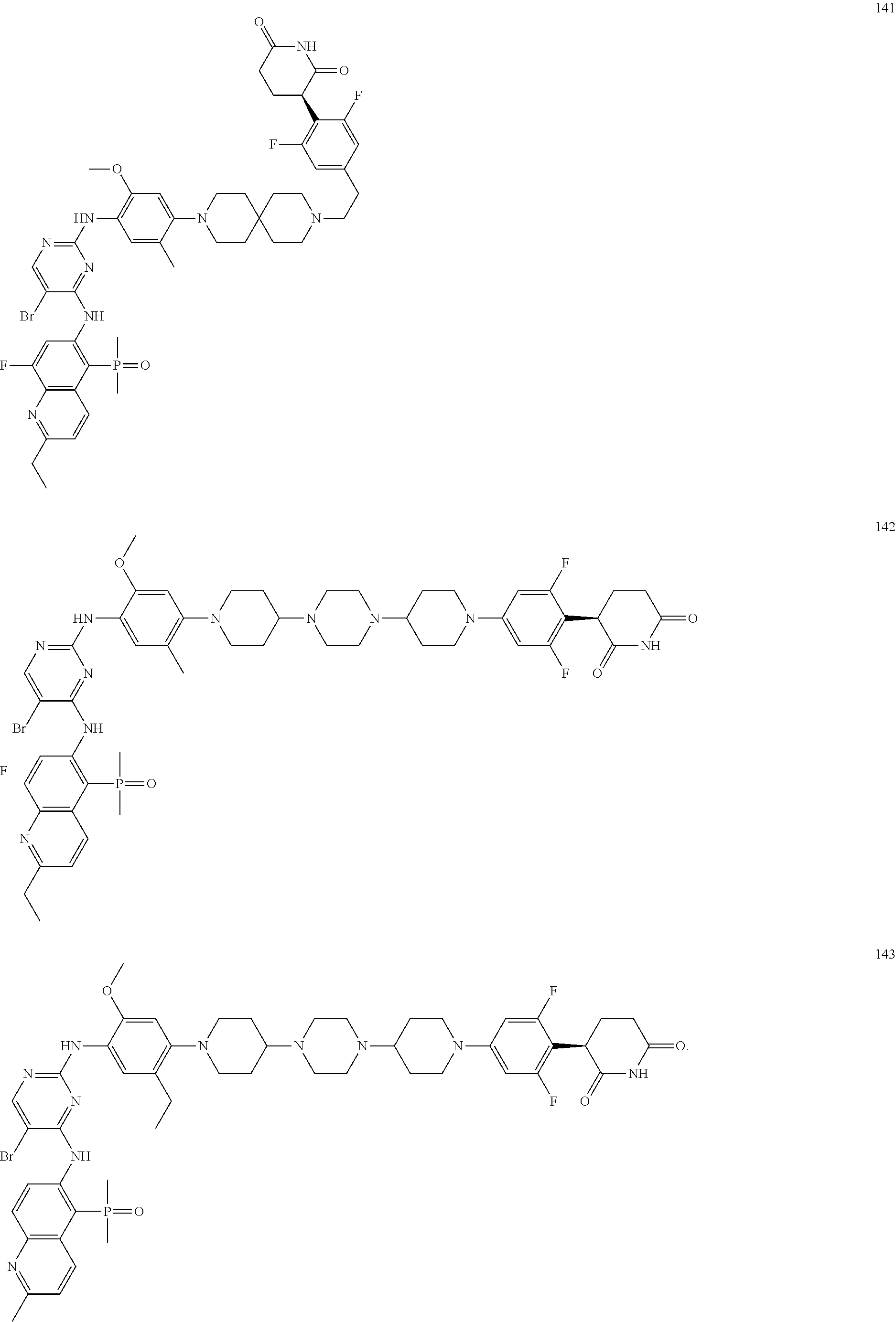

24. A pharmaceutical composition comprising a compound of any one of claims 1-23 or a pharmaceutically acceptable salt, stereoisomer, tautomer or prodrug thereof, together with a pharmaceutically acceptable excipient.

25. A method of treating a disease that can be affected by EGFR modulation, comprises administrating a subject in need thereof an effective amount of a compound of any one of claims 1-23 or a pharmaceutically acceptable salt, stereoisomer, tautomer or prodrug thereof.

26. The method of claim 25, wherein the disease is selected from cancer, preferred pancreatic cancer, breast cancer, glioblastoma multiforme, head and neck cancer, or non-small cell lung cancer.

27. Use of a compound of any one of claims 1-23 or a pharmaceutically acceptable salt, stereoisomer, tautomer or prodrug thereof in the preparation of a medicament for treating a disease that can be affected by EGFR modulation.

28. The use of claim 27, wherein the disease is cancer, preferred pancreatic cancer, breast cancer, glioblastoma multiforme, head and neck cancer, or non-small cell lung cancer.
